(19) United States
(12) Patent Application Publication
(10) Pub. No.: US 2025/0270213 A1
(43) Pub. Date: Aug. 28, 2025

(54) PI3Ka INHIBITORS

(71) Applicant: Synnovation Therapeutics, Inc., Wilmington, DE (US)

(72) Inventors: Chao Qi, Wilmington, DE (US); Lin You, Wilmington, DE (US); Fei Zhou, Wilmington, DE (US); Yu Li, Wilmington, DE (US); Hewen Zheng, Wilmington, DE (US); Yu Bai, Wilmington, DE (US); Jun Pan, Wilmington, DE (US); Liangxing Wu, Wilmington, DE (US); Wenqing Yao, Wilmington, DE (US)

(21) Appl. No.: 18/857,559

(22) PCT Filed: Apr. 19, 2023
(86) PCT No.: PCT/US2023/065937

§ 371 Date: Oct. 17, 2024

Related U.S. Application Data
(60) Provisional application No. 63/439,734, filed on Jan. 18, 2023.
(60) Provisional application No. 63/426,139, filed on Nov. 17, 2022.
(60) Provisional application No. 63/405,050, filed on Sep. 9, 2022.
(60) Provisional application No. 63/333,104, filed on Apr. 20, 2022.

(51) Int. Cl. C07D 487/04; C07D 471/04; C07D 471/16

(52) U.S. Cl. CPC C07D 487/04; C07D 471/04; C07D 471/16

(57) ABSTRACT

The present disclosure provides compounds, compositions, and methods useful for inhibiting PI3Kα, and/or treating a disease, disorder, or condition associated with PI3Kα, and/or treating cancer.

### TECHNICAL FIELD

The present disclosure provides compounds as well as their pharmaceutical compositions that modulate the activity of PI3Kα and are useful in the treatment of various diseases related to PI3Kα, including cancer.

### BACKGROUND

In the past few decades, signal transduction events have been studied to demonstrate critical roles in regulating almost all aspects of biological responses. Aberrant activation of the signaling pathways regulating cell survival and proliferation is commonly observed in many human cancers. The phosphoinositide 3-kinases (PI3Ks) signaling pathway is documented to be one of the highly mutated pathways in human cancers (Vogelstein et al., Science, 2013, 339(6127), 1546-1558). The PI3K signaling pathway regulates cell survival and proliferation. Increased activity of this pathway is associated with tumor progression and resistance to cancer therapies (Fusco et al., Front Oncol., 2021, 11, 644737).

PI3Ks belong to a lipid kinase family which catalyzes the phosphorylation of lipids contained in or associated with cell membranes. The PI3K family has fifteen kinases with distinct substrates, expression pattern, and modes of regulation. The class-I PI3Ks (p110α, p110β, p110δ, and p110γ) are typically activated by tyrosine receptor kinases or G-protein coupled receptors to generate PIP3, which activates downstream effectors of Akt, mTOR, or Rho GTPases (Fruman et al., Nat. Rev. Drug Discov., 2014, 13(2), 140-156).

Genetic mutations in the gene coding for PI3Kα are hotspot point mutations within helical and kinase domains, such as E542K, E545K and H1047R. These mutations have been observed to occur in many cancer types such as lung, stomach, endometrial, ovarian, bladder, breast, colon, brain, prostate, and skin cancers. Because these gain-of-function mutations in PI3Kα are associated with tumor progression, targeting this pathway may provide valuable therapeutic opportunities (Courtney et al., J. Clin. Oncol., 2010, 28 (6), 1075-1083). While multiple inhibitors of PI3Ks have been developed (for example, taselisib, alpelisib, buparlisib and others), these molecules inhibit multiple PI3K isoforms. These “pan-PI3K” inhibitors have encountered major hurdle in the clinical development due to inability to achieve the required level of target inhibition in tumors while avoiding toxicity in cancer patients (Fruman et al., Nat. Rev. Drug Discov., 2014, 13(2), 140-156). The toxicity of PI3K inhibitors is dependent on their isoform selectivity profile. Inhibition of PI3Kα is associated with hyperglycemia and rash, while inhibition of PI3Kδ or PI3Kγ is associated with diarrhea, myelosuppression, and transaminitis (Hanker et al., Cancer Discov., 2019, 9(4), 482-491). Therefore, selective inhibitors of PI3Kα may increase the therapeutic window, enabling sufficient target inhibition in the tumor while avoiding dose-limiting toxicity in cancer patients. However, given the central role of PI3Kα in regulating glucose homeostasis and other critical physiological process, current PI3Kα selective inhibitors, which are equally potent to wild-type and mutant PI3Kα, often cause hyperglycemia and/or hyperinsulinemia (Busaidy et al., J. Clin. Oncol., 2012, 30, 2919-2928). In summary, developing inhibitors with enhanced selectivity for mutant PI3Kα against wild-type PI3Kα would be able to overcome the problem of compensatory insulin production and hyperglycemia.

### SUMMARY

The present disclosure provides compounds and/or compositions useful for inhibiting PI3Kα. In some embodiments, provided compounds and/or compositions are useful for selectively inhibiting PI3Kα over other PI3K isoforms. In some embodiments, provided compounds and/or compositions are useful for selectively inhibiting mutant PI3Kα over wide-type PI3Kα. In some embodiments, provided compounds and/or compositions are useful, among other things, treating and/or preventing diseases, disorders, or conditions associated with PI3Kα. In some embodiments, provided compounds and/or compositions are useful, among other things, treating and/or preventing diseases, disorders, or conditions associated with mutant PI3Kα.

In some embodiments, the present disclosure provides certain compounds and/or compositions that are useful in medicine, and particularly for treating cancer.

In some embodiments, the present disclosure provides a compound of Formula I:

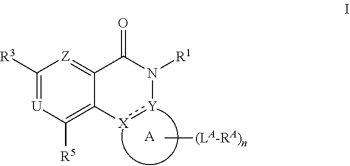

or a pharmaceutically acceptable salt thereof, wherein each of X, Y, Z, U, Ring A, L^{A}, R^{A}, R¹, R³, R⁵, and n is as defined herein.

In some embodiments, provided compounds have structures of any of Formulae II, III, IV, V, Va, VI, VIa, VII, VIIa, and VIIb as described herein.

In some embodiments, provided compounds have structures of any of Formulae II, III, IV, V, Va, VI, and VIa as described herein.

In some embodiments, the present disclosure provides compositions that comprise and/or deliver a provided compound. In some embodiments, such compositions are pharmaceutical compositions comprising a pharmaceutically acceptable carrier.

The present disclosure further provides methods of inhibiting PI3Kα activity, comprising contacting the PI3Kα with a compound described herein, or a pharmaceutically acceptable salt thereof.

The present disclosure further provides methods of treating a disease or a disorder associated with PI3Kα in a patient by administering to the patient a therapeutically effective amount of a compound of the disclosure, or a pharmaceutically acceptable salt thereof.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

### DETAILED DESCRIPTION

### Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomic, and geometric (or conformational) mixtures of provided compounds are within the scope of the disclosure. For example, in some case, Table 1 shows one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. The isotopically-labeled compounds may have one or more atoms replaced by an atom having an atomic mass or mass number usually found in nature. Examples of isotopes present in compounds of the present disclosure include isotopes of hydrogen, carbon, nitrogen, oxygen, phosphorus, fluorine and chlorine, such as, but not limited to, ²H, ³H, ¹³C, ¹⁴C, ¹⁵N, ¹⁷O, ¹⁸O, ³⁵S and ¹⁸F. Certain isotopically-labeled compounds of the present disclosure, in addition to being useful as therapeutic agents, are also useful in drug and/or substrate tissue distribution assays, as analytical tools or as probes in other biological assays. In one aspect of the present disclosure, tritiated (e.g., ³H) and carbon-14 (e.g., ¹⁴C) isotopes are useful given their ease of detectability. In another aspect of the present invention, replacement of one or more hydrogen atoms with heavier isotopes such as deuterium, (e.g., ²H) can afford certain therapeutic advantages.

As used herein and unless otherwise specified, the suffix “-ene” is used to describe a bivalent group. Thus, any of the terms above can be modified with the suffix “-ene” to describe a bivalent version of that moiety. For example, a bivalent carbocycle is “carbocyclylene”, a bivalent aryl ring is “arylene”, a bivalent benzene ring is “phenylene”, a bivalent heterocycle is “heterocyclylene”, a bivalent heteroaryl ring is “heteroarylene”, a bivalent alkyl chain is “alkylene”, a bivalent alkenyl chain is “alkenylene”, a bivalent alkynyl chain is “alkynylene”, and so forth.

Aliphatic: As used herein, the term “aliphatic” refers to a straight-chain (i.e., unbranched) or branched, optionally substituted hydrocarbon chain that is completely saturated or that contains one or more units of unsaturation, or a monocyclic or bicyclic hydrocarbon that is completely saturated or that contains one or more units of unsaturation but which is not aromatic (also referred to herein as “carbocyclic” or “cycloaliphatic”), that, unless otherwise specified, has a single point of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, alkynyl groups and hybrids thereof. In some embodiments, “aliphatic” refers to a straight-chain (i.e., unbranched) or branched, optionally substituted hydrocarbon chain that is completely saturated or that contains one or more units of unsaturation that has a single point of attachment to the rest of the molecule.

Alkyl: The term “alkyl”, used alone or as part of a larger moiety, refers to a saturated optionally substituted straight or branched hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkenyl: The term “alkenyl”, used alone or as part of a larger moiety, refers to an optionally substituted straight or branched hydrocarbon chain having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Aryl: As used herein, the term “aryl” refers to monocyclic, bicyclic, and polycyclic ring systems having a total of six to fourteen ring members (e.g., C₆₋₁₄), wherein at least one ring in the system is aromatic and wherein each ring in the system contains three to seven ring members. The term “aryl” may be used interchangeably with the term “aryl ring”. In some embodiments, “aryl” refers to an aromatic ring system which includes, but not limited to, phenyl, naphthyl, anthracyl and the like, which may bear one or more substituents. Unless otherwise specified, “aryl” groups are hydrocarbons.

Bivalent: As used herein, the term “bivalent” refers to a chemical moiety with two points of attachment to the rest of the molecule. For example, “bivalent C₁₋₆ aliphatic,” refers to bivalent aliphatic groups that are as defined herein, containing 1-6 aliphatic carbon atoms.

Carbocyclyl: As used herein, the terms “carbocyclyl,” “carbocycle,” and “carbocyclic ring” refer to saturated or partially unsaturated cyclic aliphatic monocyclic, bicyclic, or polycyclic ring systems, as described herein, having from 3 to 14 members, wherein the aliphatic ring system is optionally substituted as described herein. Carbocyclic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, “carbocyclyl” (or “cycloaliphatic”) refers to an optionally substituted monocyclic C₃-C₈ hydrocarbon, or an optionally substituted C₅-C₁₀ bicyclic hydrocarbon that is completely saturated or that contains one or more units of unsaturation, but which is not aromatic, that has a single point of attachment to the rest of the molecule. The term “cycloalkyl” refers to an optionally substituted saturated ring system of about 3 to about 10 ring carbon atoms. In some embodiments, cycloalkyl groups have 3-6 carbons. Exemplary monocyclic cycloalkyl rings include cyclopropyl, cyclobutyl, cyclopentyl, cyclohexyl, and cycloheptyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Carrier: As used herein, the term “carrier” refers to a diluent, adjuvant, excipient, or vehicle with which a composition is administered. In some embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Heteroaryl: As used herein, the terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π electrons shared in a cyclic array; and having, in addition to carbon atoms, from one to five heteroatoms. Exemplary heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridonyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridinyl, thienopyrimidinyl, triazolopyridinyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzothiazolyl, benzothiadiazolyl, benzoxazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, and benzoisoxazolyl. The term “heteroaryl” may be used interchangeably with the terms “heteroaryl ring”, “heteroaryl group”, or “heteroaromatic”, any of which terms include rings that are optionally substituted.

Heteroatom: As used herein, the term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, and “heterocyclic ring” are used interchangeably and refer to a stable 3- to 8-membered monocyclic or 5- to 10-membered bicyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl, heteroaryl, or cycloaliphatic rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)).

Partially Unsaturated: As used herein, the term “partially unsaturated”, when referring to a ring moiety, means a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: As used herein, the term “pharmaceutically acceptable salt” refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977).

Prevent or prevention: As used herein, the term “prevent” or “prevention,” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Substituted or optionally substituted: As described herein, compounds of this disclosure may contain “optionally substituted” moieties. In general, the term “substituted,” whether preceded by the term “optionally” or not, means that one or more hydrogens of the designated moiety are replaced with a suitable substituent (i.e., as described below for optionally substituted groups). “Substituted” applies to one or more hydrogens that are either explicit or implicit from the structure (e.g.,

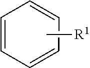

refers to at least

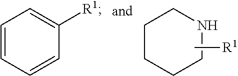

refers to at least

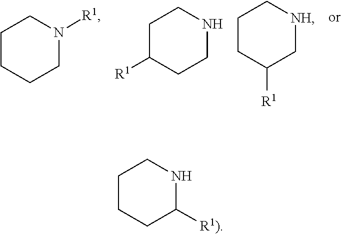

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)(═NR^{o})R^{o}; —(CH₂)₀₋₄S(O)₂OR^{o}; —(CH₂)₀₋₄OS(O)₂R^{o}; —(CH₂)₀₋₄—S(O)₂NR^{o}₂; —(CH₂)₀₋₄S(O)(═NR^{o})NR^{o}₂; —(CH₂)₀₋₄S(O)R^{o}; —N(R^{o})S(O)₂NR^{o}₂; —N(R^{o})S(O)₂R^{o}; —N(R^{o})S(O)(═NR^{o})R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; —SiR^{o}₃; —(C₁₋₄ straight or branched alkylene)O—N(R^{o}₂; or —(C₁₋₄ straight or branched alkylene)C(O)O—N(R^{o}₂, wherein each R^{o} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, —O(haloR^{●}), —(CH₂)₀₋₂CN, —N₃, —(CH₂)₀₋₂C(O)R^{●}, —(CH₂)₀₋₂C(O)OH, —(CH₂)₀₋₂C(O)OR^{●}, —(CH₂)₀₋₂C(O)NH₂, —(CH₂)₀₋₂C(O)NHR^{●}, —(CH₂)₀₋₂C(O)NR^{●}₂, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —(CH₂)₀₋₂NHC(O)R^{●}, —(CH₂)₀₋₂NR^{●}C(O)R^{●}, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable divalent substituents on a saturated carbon atom of R^{o} include ═O and ═S. Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†}) S(O)₂R^{†}; wherein each R^{†} is independently hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Treat: As used herein, the term “treat” (also “treatment” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition.

### Provided Compounds

In some embodiments, the present disclosure provides a compound of Formula I:

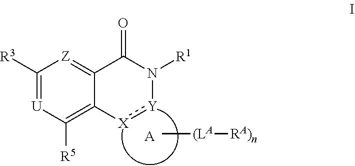

or a pharmaceutically acceptable salt thereof, wherein:
   [symbol] is a single or double bond;
   X is N or C;
   Y is N or C;
   Ring A is phenyl, 5- to 7-membered saturated or partially unsaturated monocyclic carbocyclyl, 5- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur;
   each L^{A} is independently a covalent bond or optionally substituted bivalent C₁₋₆ aliphatic;
   each R^{A} is independently oxo, halogen, —CN, —OR^{A1}, —SR^{A1}, —N(R^{A1})₂, —C(O)R^{A1}, —C(O)OR^{A1}, —C(O)N(R^{A1})₂, —C(O)NR^{A1}(OR^{A1}), —OC(O)R^{A1}—OC(O)N(R^{A1})₂, —OC(O)OR^{A1}, —OSO₂R^{A1}, —OSO₂N(R^{A1})₂, —N(R^{A1})C(O)R^{A1}, —NR^{A1}C(O)OR^{A1}, —NR^{A1}C(O)N(R^{A1})₂, —N(R^{A1})SO₂R^{A1}, —NR^{A1}S(O)₂N(R^{A1})₂, —NR^{A1}OR^{A1}, —NR^{A1}S(O)R^{A1}, —NR^{A1}S(O)N(R^{A1})₂, —S(O)R^{A1}, —SO₂R^{A1}, —S(O)N(R^{A1})₂, —SO₂N(R^{A1})₂, —SO₃R^{A1}, —C(═NR^{m})R^{A1}, —C(═NR^{m})N(R^{A1})₂, —NR^{A1}C(═NR^{m})R^{A1}, —NR^{A1}C(═NR^{m})N(R^{A1})₂, —NR^{A1}S(O)(═NR^{m})R^{A1}, —NR^{A1}S(O)(═NR^{m})N(R^{A1})₂, —OS(O)(═NR^{m})R^{A1}, —S(O)(═NR^{m})R^{A1}, —S(O)(═NR^{m})N(R^{A1})₂, —P(O)(R^{A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
   wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents;
   or two L^{A}-R^{A} taken together with the atoms to which they are attached form a 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl is independently optionally substituted with 1, 2, 3, 4, 5, or 6 R^{AG} substituents;
   R¹ is hydrogen or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur;
   or R¹ and one of L^{A}-R^{A} taken together with the atoms to which they are attached form a 3- to 7-membered saturated or partially unsaturated monocyclic heterocyclyl having 1-3 heteroatoms independently selected from nitrogen, oxygen, and sulfur, or 5- to 14-membered saturated or partially unsaturated bicyclic heterocyclyl having 1-4 heteroatoms independently selected from nitrogen, oxygen, and sulfur, wherein each of the 3- to 7-membered monocyclic heterocyclyl and 5- to 14-membered bicyclic heterocyclyl is independently optionally substituted with 1, 2, 3, 4, 5, or 6 R^{AG} substituents;
   Z is N or CR²;
   U is N or CR⁴;
   R² and R⁴ are each independently hydrogen, halogen, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur;
   R³ is —F, —Cl, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur;
   R⁵ is -L⁵-R^{5A},
   L⁵ is a covalent bond or optionally substituted bivalent C₁₋₆ aliphatic;
   or L⁵ and one of L^{A}-R^{A} taken together with the atoms to which they are attached form a 3- to 16-membered saturated or partially unsaturated monocyclic carbocyclyl, 5- to 16-membered saturated or partially unsaturated bicyclic carbocyclyl, 3- to 16-membered saturated or partially unsaturated monocyclic heterocyclyl having 1-3 heteroatoms independently selected from nitrogen, oxygen, and sulfur, or 5- to 16-membered saturated or partially unsaturated bicyclic heterocyclyl having 1-4 heteroatoms independently selected from nitrogen, oxygen, and sulfur, wherein each of the 3- to 16-membered monocyclic carbocyclyl, 5- to 16-membered bicyclic carbocyclyl, 3- to 16-membered monocyclic heterocyclyl, and 5- to 16-membered bicyclic heterocyclyl is independently optionally substituted with 1, 2, 3, 4, 5, or 6 R^{AG} substituents;
   R^{5A} is —OR^{5A11}, —SR^{5A1}, —NHR^{5A12}, —N(R^{5A12})₂, —C(O)R^{5A1}, —C(O)N(R^{5A1})₂, —C(O)NR^{5A1}(OR^{5A1}), —OC(O)R^{5A1}, —OC(O)N(R^{5A1})₂, —OC(O)OR^{5A1}, —OSO₂R^{5A1}, —OSO₂N(R^{5A1})₂, —N(R^{5A1})C(O)R^{5A1}, —NR^{5A1}C(O)OR^{5A1}, —NR^{5A1}C(O)N(R^{5A1})₂, —N(R^{5A1})SO₂R^{5A1}, —NR^{5A1}S(O)₂N(R^{5A1})₂, —NR^{5A1}OR^{5A1}, —NR^{5A1}S(O)R^{5A1}, —NR^{5A1}S(O)N(R^{5A1})₂, —S(O)R^{5A1}, —SO₂R^{5A1}, —S(O)N(R^{5A1})₂, —SO₂N(R^{5A1})₂, —SO₃R^{5A1}, —C(═NR^{m})R^{5A1}, —C(═NR^{m})N(R^{5A1})₂, —NR^{5A1}C(═NR^{m})R^{5A1}, —NR^{5A1}C(═NR^{m})N(R^{5A1})₂, —NR^{5A1}S(O)(═NR^{m})R^{5A1}, —NR^{5A1}S(O)(═NR^{m})N(R^{5A1})₂, —OS(O)(═NR^{m})R^{5A1}, —S(O)(═NR^{m})R^{5A1}, —S(O)(═NR^{m})N(R^{5A1})₂, —P(O)(R^{5A1})₂, methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
   wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A} is independently optionally substituted with 1, 2, 3, 4, 5, or 6 R^{5AG} substituents; or the methyl of R^{5A} is substituted with 1, 2, or 3 R^{5AG} substituents;
   R^{A1} and R^{5A1} are each independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
   wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A1} or R^{5A1} is independently optionally substituted with 1, 2, 3, 4, 5, or 6 R^{G1} substituents;
   or two R^{A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
   or two R^{5A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
   R^{5A11} is methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
   wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A11} is independently optionally substituted with 1, 2, 3, 4, 5, or 6 R^{G1} substituents; or
   the methyl of R^{5A11} is substituted with 1, 2, or 3 R^{G1} substituents;
   each R^{5A12} is independently C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
   wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A12} is independently optionally substituted with 1, 2, 3, 4, 5, or 6 R^{G1} substituents;
   or two R^{5A12} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
   R^{AG}, R^{5AG}, and R^{G1} are each independently halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR¹)N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
   each R is independently hydrogen or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or
   two R when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
   each R^{m} is independently —OH, —CN, or R; and
   n is 0, 1, 2, 3, or 4.

In some embodiments of the previous embodiment, L⁵ and one of L^{A}-R^{A} taken together with the atoms to which they are attached form a 3- to 7-membered saturated or partially unsaturated monocyclic carbocyclyl, 5- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, or 5- to 10-membered saturated or partially unsaturated bicyclic heterocyclyl having 1-4 heteroatoms independently selected from nitrogen, oxygen, and sulfur, wherein each of the 3- to 7-membered monocyclic carbocyclyl, 5- to 10-membered bicyclic carbocyclyl, 3- to 7-membered monocyclic heterocyclyl, and 5- to 10-membered bicyclic heterocyclyl is independently optionally substituted with 1, 2, 3, 4, 5, or 6 R^{AG} substituents.

In some embodiments:
[symbol] is a single or double bond;
X is N or C;
Y is N or C;
Ring A is phenyl, 5- to 7-membered saturated or partially unsaturated monocyclic carbocyclyl, 5- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur;
each L^{A} is independently a covalent bond or a bivalent C₁₋₆ aliphatic, wherein the bivalent C₁₋₆ aliphatic is optionally substituted with 1, 2, 3, or 4 independently selected R^{LA1} substituents;
each R^{A} is independently oxo, halogen, —CN, —OR^{A1}, —SR^{A1}, —N(R^{A1})₂, —C(O)R^{A1}, —C(O)OR^{A1}, —C(O)N(R^{A1})₂, —C(O)NR^{A1}(OR^{A1}), —OC(O)R^{A1}—OC(O)N(R^{A1})₂, —OC(O)OR^{A1}, —OSO₂R^{A1}, —OSO₂N(R^{A1})₂, —N(R^{A1})C(O)R^{A1}, —NR^{A1}C(O)OR^{A1}, —NR^{A1}C(O)N(R^{A1})₂, —N(R^{A1})SO₂R^{A1}, —NR^{A1}S(O)₂N(R^{A1})₂, —NR^{A1}OR^{A1}, —NR^{A1}S(O)R^{A1}, —NR^{A1}S(O)N(R^{A1})₂, —S(O)R^{A1}, —SO₂R^{A1}, —S(O)N(R^{A1})₂, —SO₂N(R^{A1})₂, —SO₃R^{A1}, —C(═NR^{m})R^{A1}, —C(═NR^{m})N(R^{A1})₂, —NR^{A1}C(═NR^{m})R^{A1}, —NR^{A1}C(═NR^{m})N(R^{A1})₂, —NR^{A1}S(O)(═NR^{m})R^{A1}, —NR^{A1}S(O)(═NR^{m})N(R^{A1})₂, —OS(O)(═NR^{m})R^{A1}, —S(O)(═NR^{m})R^{A1}, —S(O)(═NR^{m})N(R^{A1})₂, —P(O)(R^{A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents;
or two L^{A}-R^{A} taken together with the atoms to which they are attached form a 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl is independently optionally substituted with 1, 2, 3, 4, 5, or 6 R^{AG} substituents;
R¹ is hydrogen or a group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl are each optionally substituted with 1, 2, 3, or 4 independently selected R^{1A} substituents;
or R¹ and one of L^{A}-R^{A} taken together with the atoms to which they are attached form a 3- to 7-membered saturated or partially unsaturated monocyclic heterocyclyl having 1-3 heteroatoms independently selected from nitrogen, oxygen, and sulfur, or 5- to 14-membered saturated or partially unsaturated bicyclic heterocyclyl having 1-4 heteroatoms independently selected from nitrogen, oxygen, and sulfur, wherein each of the 3- to 7-membered monocyclic heterocyclyl and 5- to 14-membered bicyclic heterocyclyl is independently optionally substituted with 1, 2, 3, 4, 5, or 6 R^{AG} substituents;
Z is N or CR²;
U is N or CR⁴;
R² and R⁴ are each independently hydrogen, halogen, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl are each optionally substituted with 1, 2, 3, or 4 independently selected R^{2A} substituents;
R³ is —F, —Cl, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl are each optionally substituted with 1, 2, 3, or 4 independently selected R^{3A} substituents;
R⁵ is -L⁵-R^{5A};
L⁵ is a covalent bond or a bivalent C₁₋₆ aliphatic, wherein the bivalent C₁₋₆ aliphatic is optionally substituted with 1, 2, 3, or 4 independently selected R^{L5A} substituents;
or L⁵ and one of L^{A}-R^{A} taken together with the atoms to which they are attached form a 3- to 16-membered saturated or partially unsaturated monocyclic carbocyclyl, 5- to 16-membered saturated or partially unsaturated bicyclic carbocyclyl, 3- to 16-membered saturated or partially unsaturated monocyclic heterocyclyl having 1-3 heteroatoms independently selected from nitrogen, oxygen, and sulfur, or 5- to 16-membered saturated or partially unsaturated bicyclic heterocyclyl having 1-4 heteroatoms independently selected from nitrogen, oxygen, and sulfur, wherein each of the 3- to 16-membered monocyclic carbocyclyl, 5- to 16-membered bicyclic carbocyclyl, 3- to 16-membered monocyclic heterocyclyl, and 5- to 16-membered bicyclic heterocyclyl is independently optionally substituted with 1, 2, 3, 4, 5, or 6 R^{AG} substituents;
R^{5A} is —OR^{5A11}, —SR^{5A1}, —NHR^{5A12}, —N(R^{5A12})₂, —C(O)R^{5A1}, —C(O)N(R^{5A1})₂, —C(O)NR^{5A1} (OR^{5A1}), —OC(O)R^{5A1}, —OC(O)N(R^{5A1})₂, —OC(O)OR^{5A1}, —OSO₂R^{5A1}, —OSO₂N(R^{5A1})₂, —N(R^{5A1})C(O)R^{5A1}, —NR^{5A1}C(O)OR^{5A1}, —NR^{5A1}C(O)N(R^{5A1})₂, —N(R^{5A1})SO₂R^{5A1}, —NR^{5A1}S(O)₂N(R^{5A1})₂, —NR^{5A1}OR^{5A1}, —NR^{5A1}S(O)R^{5A1}, —NR^{5A1}S(O)N(R^{5A1})₂, —S(O)R^{5A1}, —SO₂R^{5A1}, —S(O)N(R^{5A1})₂, —SO₂N(R^{5A1})₂, —SO₃R^{5A1}, —C(═NR^{m})R^{5A1}, —C(═NR^{m})N(R^{5A1})₂, —NR^{5A1}C(═NR^{m})R^{5A1}, —NR^{5A1}C(═NR^{m})N(R^{5A1})₂, —NR^{5A1}S(O)(═NR^{m})R^{5A1}, —NR^{5A1}S(O)(═NR^{m})N(R^{5A1})₂, —OS(O)(═NR^{m})R^{5A1}, —S(O)(═NR^{m})R^{5A1}, —S(O)(═NR^{m})N(R^{5A1})₂, —P(O)(R^{5A1})₂, methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A} is independently optionally substituted with 1, 2, 3, 4, 5, or 6 R^{5AG} substituents; or
the methyl of R^{5A} is substituted with 1, 2, or 3 R^{5AG} substituents;
R^{A1} and R^{5A1} are each independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A1} or R^{5A1} is independently optionally substituted with 1, 2, 3, 4, 5, or 6 R^{G1} substituents;
or two R^{A1} when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{A11} substituents;
or two R^{5A1} when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{A11} substituents;
R^{5A11} is methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A11} is independently optionally substituted with 1, 2, 3, 4, 5, or 6 R^{G1} substituents; or
the methyl of R^{5A11} is substituted with 1, 2, or 3 R^{G1} substituents;
each R^{5A12} is independently C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A12} is independently optionally substituted with 1, 2, 3, 4, 5, or 6 R^{G1} substituents;
or two R^{5A12} when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{5B12} substituents;
R^{AG}, R^{5AG}, and R^{G1} are each independently oxo, halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl, 5- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{AG1} substituents;
each R is independently selected from oxo, hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl, 5- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{N} substituents; or
two R when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{N} substituents;
each R^{LA1}, R^{1A}, R^{2A}, R^{3A}, R^{L5A}, R^{A11}, R^{5B12}, R^{AG1}, and R^{N} is independently selected from oxo, halogen, —(CH₂)₀₋₄R^{o}, —(CH₂)₀₋₄OR^{o}, —O(CH₂)₀₋₄R^{o}, —O—(CH₂)₀₋₄C(O)OR^{o}, —(CH₂)₀₋₄CH(OR^{o})₂, —(CH₂)₀₋₄SR^{o}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{o}₂, —(CH₂)₀₋₄N(R^{o})C(O)R^{o}, —N(R^{o})C(S)R^{o}, —(CH₂)₀₋₄N(R^{o})C(O)NR^{o}₂, —N(R^{o})C(S)NR^{o}₂, —(CH₂)₀₋₄N(R^{o})C(O)OR^{o}, —N(R^{o})N(R^{o})C(O)R^{o}, —N(R^{o})N(R^{o})C(O)NR^{o}₂, —N(R^{o})N(R^{o})C(O)OR^{o}, —(CH₂)₀₋₄C(O)R^{o}, —C(S)R^{o}, —(CH₂)₀₋₄C(O)OR^{o}, —(CH₂)₀₋₄C(O)SR^{o}, —(CH₂)₀₋₄C(O)OSiR^{o}₃, —(CH₂)₀₋₄OC(O)R^{o}, —OC(O)(CH₂)₀₋₄SR^{o}, —(CH₂)₀₋₄SC(O)R^{o}, —(CH₂)₀₋₄C(O)NR^{o}₂, —C(S)NR^{o}₂, —C(S)SR^{o}, —SC(S)SR^{o}, —(CH₂)₀₋₄OC(O)NR^{o}₂, —C(O)N(OR^{o})R^{o}, —C(O)C(O)R^{o}, —C(O)CH₂C(O)R^{o}, —C(NOR^{o})R^{o}, —(CH₂)₀₋₄SSR^{o}, —(CH₂)₀₋₄S(O)₂R^{o}, —(CH₂)₀₋₄S(O)(═NR^{o})R^{o}, —(CH₂)₀₋₄S(O)₂OR^{o}, —(CH₂)₀₋₄OS(O)₂R^{o}, —(CH₂)₀₋₄—S(O)₂NR^{o}₂, —(CH₂)₀₋₄S(O)(═NR^{o})NR^{o}₂, —(CH₂)₀₋₄S(O)R^{o}, —N(R^{o})S(O)₂NR^{o}₂, —N(R^{o})S(O)₂R^{o}, —N(R^{o})S(O)(═NR^{o})R^{o}, —N(OR^{o})R^{o}, —C(NH)NR^{o}₂, —P(O)₂R^{o}, —P(O)R^{o}₂, —OP(O)R^{o}₂, —OP(O)(OR^{o})₂, —SiR^{o}₃, —(C₁₋₄ straight or branched alkylene)O—N(R^{o})₂, and —(C₁₋₄ straight or branched alkylene)C(O)O—N(R^{o})₂;
each R^{o} is independently oxo, hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur;
or two independent occurrences of R^{o}, taken together with their intervening atoms, form a 3- to 12-membered saturated, partially unsaturated, or aryl mono- or bicyclic ring having 0-4 heteroatoms independently selected from nitrogen, oxygen, or sulfur;
each R^{m} is independently oxo, —OH, —CN, or R; and
n is 0, 1, 2, 3, or 4.

In some embodiments of the previous embodiment:
R^{AG}, R^{5AG}, and R^{G1} are each independently halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl, 5- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{AG1} substituents;
each R is independently selected from hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl, 5- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{N} substituents; or
two R when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{N} substituents;
each R^{LA1}, R^{1A}, R^{2A}, R^{3A}, R^{L5A}, R^{A11}, R^{5B12}, R^{AG1}, and R^{N} is independently selected from halogen, —(CH₂)₀₋₄R^{o}, —(CH₂)₀₋₄OR^{o}, —O(CH₂)₀₋₄R^{o}, —O—(CH₂)₀₋₄C(O)OR^{o}, —(CH₂)₀₋₄CH(OR^{o})₂, —(CH₂)₀₋₄SR^{o}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{o}₂, —(CH₂)₀₋₄N(R^{o})C(O)R^{o}, —N(R^{o})C(S)R^{o}, —(CH₂)₀₋₄N(R^{o})C(O)NR^{o}₂, —N(R^{o})C(S)NR^{o}₂, —(CH₂)₀₋₄N(R^{o})C(O)OR^{o}, —N(R^{o})N(R^{o})C(O)R^{o}, —N(R^{o})N(R^{o})C(O)NR^{o}₂, —N(R^{o})N(R^{o})C(O)OR^{o}, —(CH₂)₀₋₄C(O)R^{o}, —C(S)R^{o}, —(CH₂)₀₋₄C(O)OR^{o}, —(CH₂)₀₋₄C(O)SR^{o}, —(CH₂)₀₋₄C(O)OSiR^{o}₃, —(CH₂)₀₋₄OC(O)R^{o}, —OC(O)(CH₂)₀₋₄SR^{o}, —(CH₂)₀₋₄SC(O)R^{o}, —(CH₂)₀₋₄C(O)NR^{o}₂, —C(S)NR^{o}₂, —C(S)SR^{o}, —SC(S)SR^{o}, —(CH₂)₀₋₄OC(O)NR^{o}₂, —C(O)N(OR^{o})R^{o}, —C(O)C(O)R^{o}, —C(O)CH₂C(O)R^{o}, —C(NOR^{o})R^{o}, —(CH₂)₀₋₄SSR^{o}, —(CH₂)₀₋₄S(O)₂R^{o}, —(CH₂)₀₋₄S(O)(═NR^{o})R^{o}, —(CH₂)₀₋₄S(O)₂OR^{o}, —(CH₂)₀₋₄OS(O)₂R^{o}, —(CH₂)₀₋₄—S(O)₂NR^{o}₂, —(CH₂)₀₋₄S(O)(═NR^{o})NR^{o}₂, —(CH₂)₀₋₄S(O)R^{o}, —N(R^{o})S(O)₂NR^{o}₂, —N(R^{o})S(O)₂R^{o}, —N(R^{o})S(O)(═NR^{o})R^{o}, —N(OR^{o})R^{o}, —C(NH)NR^{o}₂, —P(O)₂R^{o}, —P(O)R^{o}₂, —OP(O)R^{o}₂, —OP(O)(OR^{o}₂, —SiR^{o}₃, —(C₁₋₄ straight or branched alkylene)O—N(R^{o}₂, and —(C₁₋₄ straight or branched alkylene)C(O)O—N(R^{o})₂;
each R^{o} is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur;
or two independent occurrences of R^{o}, taken together with their intervening atoms, form a 3- to 12-membered saturated, partially unsaturated, or aryl mono- or bicyclic ring having 0-4 heteroatoms independently selected from nitrogen, oxygen, or sulfur; and
each R^{m} is independently —OH, —CN, or R.

In some embodiments of the previous embodiments, L⁵ and one of L^{A}-R^{A} taken together with the atoms to which they are attached form a 3- to 7-membered saturated or partially unsaturated monocyclic carbocyclyl, 5- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, or 5- to 10-membered saturated or partially unsaturated bicyclic heterocyclyl having 1-4 heteroatoms independently selected from nitrogen, oxygen, and sulfur, wherein each of the 3- to 7-membered monocyclic carbocyclyl, 5- to 10-membered bicyclic carbocyclyl, 3- to 7-membered monocyclic heterocyclyl, and 5- to 10-membered bicyclic heterocyclyl is independently optionally substituted with 1, 2, 3, 4, 5, or 6 R^{AG} substituents.

In some embodiments:
[symbol] is a single or double bond;
X is N or C;
Y is N or C;
Ring A is phenyl or 5- to 6-membered monocyclic heteroaryl having 1-4 heteroatoms independently selected from nitrogen, oxygen, and sulfur;
each L^{A} is independently a covalent bond or optionally substituted bivalent C₁₋₆ aliphatic;
each R^{A} is independently oxo, halogen, —CN, —OR^{A1}, —SR^{A1}, —N(R^{A1})₂, —C(O)R^{A1}, —C(O)OR^{A1}, —C(O)N(R^{A1})₂, —C(O)NR^{A1}(OR^{A1}), —OC(O)R^{A1}—OC(O)N(R^{A1})₂, —OC(O)OR^{A1}, —OSO₂R^{A1}, —OSO₂N(R^{A1})₂, —N(R^{A1})C(O)R^{A1}, —NR^{A1}C(O)OR^{A1}, —NR^{A1}C(O)N(R^{A1})₂, —N(R^{A1})SO₂R^{A1}, —NR^{A1}S(O)₂N(R^{A1})₂, —NR^{A1}OR^{A1}, —NR^{A1}S(O)R^{A1}, —NR^{A1}S(O)N(R^{A1})₂, —S(O)R^{A1}, —SO₂R^{A1}, —S(O)N(R^{A1})₂, —SO₂N(R^{A1})₂, —SO₃R^{A1}, —C(═NR^{m})R^{A1}, —C(═NR^{m})N(R^{A1})₂, —NR^{A1}C(═NR^{m})R^{A1}, —NR^{A1}C(═NR^{m})N(R^{A1})₂, —NR^{A1}S(O)(═NR^{m})R^{A1}, —NR^{A1}S(O)(═NR^{m})N(R^{A1})₂, —OS(O)(═NR^{m})R^{A1}, —S(O)(═NR^{m})R^{A1}, —S(O)(═NR^{m})N(R^{A1})₂, —P(O)(R^{A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents;
R¹ is hydrogen or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur;
Z is N or CR²;
U is N or CR⁴;
R² and R⁴ are each independently hydrogen, halogen, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur;
R³ is —F, —Cl, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur;
R⁵ is -L⁵-R^{5A};
L⁵ is a covalent bond or optionally substituted bivalent C₁₋₆ aliphatic;
R^{5A} is —OR^{5A11}, —SR^{5A1}, —NHR^{5A12}, —N(R^{5A12})₂, —C(O)R^{5A1}, —C(O)N(R^{5A1})₂, —C(O)NR^{5A1} (OR^{5A1}), —OC(O)R^{5A1}, —OC(O)N(R^{5A1})₂, —OC(O)OR^{5A1}, —OSO₂R^{5A1}, —OSO₂N(R^{5A1})₂, —N(R^{5A1})C(O)R^{5A1}, —NR^{5A1}C(O)OR^{5A1}, —NR^{5A1}C(O)N(R^{5A1})₂, —N(R^{5A1})SO₂R^{5A1}, —NR^{5A1}S(O)₂N(R^{5A1})₂, —NR^{5A1}OR^{5A1}, —NR^{5A1}S(O)R^{5A1}, —NR^{5A1}S(O)N(R^{5A1})₂, —S(O)R^{5A1}, —SO₂R^{5A1}, —S(O)N(R^{5A1})₂, —SO₂N(R^{5A1})₂, —SO₃R^{5A1}, —C(═NR^{m})R^{5A1}, —C(═NR^{m})N(R^{5A1})₂, —NR^{5A1}C(═NR^{m})R^{5A1}, —NR^{5A1}C(═NR^{m})N(R^{5A1})₂, —NR^{5A1}S(O)(═NR^{m})R^{5A1}, —NR^{5A1}S(O)(═NR^{m})N(R^{5A1})₂, —OS(O)(═NR^{m})R^{5A1}, —S(O)(═NR^{m}1)R^{5A1}, —S(O)(═NR^{m})N(R^{5A1})₂, —P(O)(R^{5A1})₂, methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A} is independently optionally substituted with 1, 2, 3, 4, 5, or 6 R^{5AG} substituents; or
the methyl of R^{5A} is substituted with 1, 2, or 3 R^{5AG} substituents;
R^{A1} and R^{5A1} are each independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A1} or R^{5A1} is independently optionally substituted with 1, 2, 3, 4, 5, or 6 R^{G1} substituents;
or two R^{A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
or two R^{5A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
R^{5A11} is methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A11} is independently optionally substituted with 1, 2, 3, 4, 5, or 6 R^{G1} substituents; or
the methyl of R^{5A11} is substituted with 1, 2, or 3 R^{G1} substituents;
each R^{5A12} is independently C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A12} is independently optionally substituted with 1, 2, 3, 4, 5, or 6 R^{G1} substituents;
or two R^{5A12} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
R^{AG}, R^{5AG}, and R^{G1} are each independently halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
each R is independently hydrogen or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or
two R when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
each R^{m} is independently —OH, —CN, or R; and
n is 0, 1, 2, 3, or 4.

In some embodiments:
[symbol] is a single or double bond;
X is N or C;
Y is N or C;
Ring A is phenyl, or 5- to 6-membered monocyclic heteroaryl having 1-4 heteroatoms independently selected from nitrogen, oxygen, and sulfur;
each L^{A} is independently a covalent bond or a bivalent C₁₋₆ aliphatic, wherein the bivalent C₁₋₆ aliphatic is optionally substituted with 1, 2, 3, or 4 independently selected R^{LA1} substituents;
each R^{A} is independently oxo, halogen, —CN, —OR^{A1}, —SR^{A1}, —N(R^{A1})₂, —C(O)R^{A1}, —C(O)OR^{A1}, —C(O)N(R^{A1})₂, —C(O)NR^{A1}(OR^{A1}), —OC(O)R^{A1}—OC(O)N(R^{A1})₂, —OC(O)OR^{A1}, —OSO₂R^{A1}, —OSO₂N(R^{A1})₂, —N(R^{A1})C(O)R^{A1}, —NR^{A1}C(O)OR^{A1}, —NR^{A1}C(O)N(R^{A1})₂, —N(R^{A1})SO₂R^{A1}, —NR^{A1}S(O)₂N(R^{A1})₂, —NR^{A1}OR^{A1}, —NR^{A1}S(O)R^{A1}, —NR^{A1}S(O)N(R^{A1})₂, —S(O)R^{A1}, —SO₂R^{A1}, —S(O)N(R^{A1})₂, —SO₂N(R^{A1})₂, —SO₃R^{A1}, —C(═NR^{m})R^{A1}, —C(═NR^{m})N(R^{A1})₂, —NR^{A1}C(═NR^{m})R^{A1}, —NR^{A1}C(═NR^{m})N(R^{A1})₂, —NR^{A1}S(O)(═NR^{m})R^{A1}, —NR^{A1}S(O)(═NR^{m})N(R^{A1})₂, —OS(O)(═NR^{m})R^{A1}, —S(O)(═NR^{m})R^{A1}, —S(O)(═NR^{m})N(R^{A1})₂, —P(O)(R^{A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents;
R¹ is hydrogen or a group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl are each optionally substituted with 1, 2, 3, or 4 independently selected R^{1A} substituents;
Z is N or CR²;
U is N or CR⁴;
R² and R⁴ are each independently hydrogen, halogen, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl are each optionally substituted with 1, 2, 3, or 4 independently selected R^{2A} substituents;
R³ is —F, —Cl, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl are each optionally substituted with 1, 2, 3, or 4 independently selected R^{3A} substituents;
R⁵ is -L⁵-R^{5A};
L⁵ is a covalent bond or a bivalent C₁₋₆ aliphatic, wherein the bivalent C₁₋₆ aliphatic is optionally substituted with 1, 2, 3, or 4 independently selected R^{L5A} substituents;
R^{5A} is —OR^{5A11}, —SR^{5A1}, —NHR^{5A12}, —N(R^{5A12})₂, —C(O)R^{5A1}, —C(O)N(R^{5A1})₂, —C(O)NR^{5A1} (OR^{5A1}), —OC(O)R^{5A1}, —OC(O)N(R^{5A1})₂, —OC(O)OR^{5A1}, —OSO₂R^{5A1}, —OSO₂N(R^{5A1})₂, —N(R^{5A1})C(O)R^{5A1}, —NR^{5A1}C(O)OR^{5A1}, —NR^{5A1}C(O)N(R^{5A1})₂, —N(R^{5A1})SO₂R^{5A1}, —NR^{5A1}S(O)₂N(R^{5A1})₂, —NR^{5A1}OR^{5A1}, —NR^{5A1}S(O)R^{5A1}, —NR^{5A1}S(O)N(R^{5A1})₂, —S(O)R^{5A1}, —SO₂R^{5A1}, —S(O)N(R^{5A1})₂, —SO₂N(R^{5A1})₂, —SO₃R^{5A1}, —C(═NR^{m})R^{5A1}, —C(═NR^{m})N(R^{5A1})₂, —NR^{5A1}C(═NR^{m})R^{5A1}, —NR^{5A1}C(═NR^{m})N(R^{5A1})₂, —NR^{5A1}S(O)(═NR^{m})R^{5A1}, —NR^{5A1}S(O)(═NR^{m})N(R^{5A1})₂, —OS(O)(═NR^{m})R^{5A1}, —S(O)(═NR^{m})R^{5A1}, —S(O)(═NR^{m})N(R^{5A1})₂, —P(O)(R^{5A1})₂, methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A} is independently optionally substituted with 1, 2, 3, 4, 5, or 6 R^{5AG} substituents; or
the methyl of R^{5A} is substituted with 1, 2, or 3 R^{5AG} substituents;
R^{A1} and R^{5A1} are each independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A1} or R^{5A1} is independently optionally substituted with 1, 2, 3, 4, 5, or 6 R^{G1} substituents;
or two R^{A1} when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{A11} substituents;
or two R^{5A1} when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{A11} substituents;
R^{5A11} is methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A11} is independently optionally substituted with 1, 2, 3, 4, 5, or 6 R^{G1} substituents; or
the methyl of R^{5A11} is substituted with 1, 2, or 3 R^{G1} substituents;
each R^{5A12} is independently C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A12} is independently optionally substituted with 1, 2, 3, 4, 5, or 6 R^{G1} substituents;
or two R^{5A12} when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{5B12} substituents;
R^{AG}, R^{5AG}, and R^{G1} are each independently halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl, 5- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{AG1} substituents;
each R is independently selected from hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl, 5- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{N} substituents; or
two R when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{N} substituents;
each R^{LA1}, R^{1A}, R^{2A}, R^{3A}, R^{L5A}, R^{A11}, R^{5B12}, R^{AG1}, and R^{N} is independently selected from halogen, —(CH₂)₀₋₄R^{o}, —(CH₂)₀₋₄OR^{o}, —O(CH₂)₀₋₄R^{o}, —O—(CH₂)₀₋₄C(O)OR^{o}, —(CH₂)₀₋₄CH(OR^{o})₂, —(CH₂)₀₋₄SR^{o}, —(CH₂)₀₋₄Ph, —(CH₂)₀₋₄O(CH₂)₀₋₁Ph, —CH═CHPh, —(CH₂)₀₋₄₀ (CH₂)₀₋₁-pyridyl, —NO₂, —CN, —N₃, —(CH₂)₀₋₄N(R^{o}₂, —(CH₂)₀₋₄N(R^{o})C(O)R^{o}, —N(R^{o})C(S)R^{o}, —(CH₂)₀₋₄N(R^{o})C(O)NR^{o}₂, —N(R^{o})C(S)NR^{o}₂, —(CH₂)₀₋₄N(R^{o})C(O)OR^{o}, —N(R^{o})N(R^{o})C(O)R^{o}, —N(R^{o})N(R^{o})C(O)NR^{o}₂, —N(R^{o})N(R^{o})C(O)OR^{o}, —(CH₂)₀₋₄C(O)R^{o}, —C(S)R^{o}, —(CH₂)₀₋₄C(O)OR^{o}, —(CH₂)₀₋₄C(O)SR^{o}, —(CH₂)₀₋₄C(O)OSiR^{o}₃, —(CH₂)₀₋₄OC(O)R^{o}, —OC(O)(CH₂)₀₋₄SR^{o}, —(CH₂)₀₋₄SC(O)R^{o}, —(CH₂)₀₋₄C(O)NR^{o}₂, —C(S)NR^{o}₂, —C(S)SR^{o}, —SC(S)SR^{o}, —(CH₂)₀₋₄OC(O)NR^{o}₂, —C(O)N(OR^{o})R^{o}, —C(O)C(O)R^{o}, —C(O)CH₂C(O)R^{o}, —C(NOR^{o})R^{o}, —(CH₂)₀₋₄SSR^{o}, —(CH₂)₀₋₄S(O)₂R^{o}, —(CH₂)₀₋₄S(O)(═NR^{o})R^{o}, —(CH₂)₀₋₄S(O)₂OR^{o}, —(CH₂)₀₋₄OS(O)₂R^{o}, —(CH₂)₀₋₄—S(O)₂NR^{o}2, —(CH₂)₀₋₄S(O)(═NR^{o})NR^{o}₂, —(CH₂)₀₋₄S(O)R^{o}, —N(R^{o})S(O)₂NR^{o}2, —N(R^{o})S(O)₂R^{o}, —N(R^{o})S(O)(═NR^{o})R^{o}, —N(OR^{o})R^{o}, —C(NH)NR^{o}₂, —P(O)₂R^{o}, —P(O)R^{o}₂, —OP(O)R^{o}₂, —OP(O)(OR^{o})₂, —SiR^{o}₃, —(C₁₋₄ straight or branched alkylene)O—N(R^{o}₂, and —(C₁₋₄ straight or branched alkylene)C(O)O—N(R^{o}₂;
each R^{o} is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur;
or two independent occurrences of R^{o}, taken together with their intervening atoms, form a 3- to 12-membered saturated, partially unsaturated, or aryl mono- or bicyclic ring having 0-4 heteroatoms independently selected from nitrogen, oxygen, or sulfur;
each R^{m} is independently —OH, —CN, or R; and
n is 0, 1, 2, 3, or 4.

In some embodiments, the present disclosure provides compounds of Formula II:

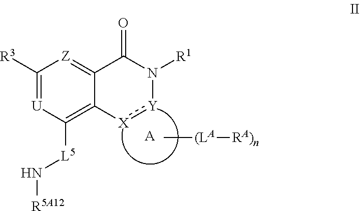

or a pharmaceutically acceptable salt thereof, wherein each of X, Y, Z, U, Ring A, L^{A}, R^{A}, R¹, R³, L⁵, R^{5A12}, and n is defined above for Formula I and described in classes and subclasses herein, both singly and in combination.

In some embodiments, the present disclosure provides compounds of Formula III:

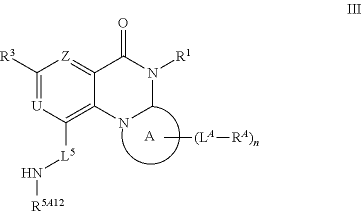

or a pharmaceutically acceptable salt thereof, wherein each of Z, U, Ring A, L^{A}, R^{A}, R¹, R³, L⁵, R^{5A12}, and n is defined above for Formula I and described in classes and subclasses herein, both singly and in combination.

As described above, in some embodiments of any of Formulae I and II, [symbol] is a single or double bond. In some embodiments, [symbol] is a single bond. In some embodiments, [symbol] is a double bond.

As described above, in some embodiments of any of Formulae I and II, X is N or C. In some embodiments, X is N. In some embodiments, X is C.

As described above, in some embodiments of any of Formulae I and II, Y is N or C. In some embodiments, Y is N. In some embodiments, Y is C.

In some embodiments, X is C and Y is N. In some embodiments, X is N and Y is C. In some embodiments, X is C and Y is C.

As described above, in some embodiments of any of Formulae I, II, and III, Ring A is phenyl, or 5- to 6-membered monocyclic heteroaryl having 1-4 heteroatoms independently selected from nitrogen, oxygen, and sulfur. In some embodiments of any of Formulae I, II, III, IV, V, and Va, Ring A is phenyl, or 5- to 6-membered monocyclic heteroaryl having 1-4 heteroatoms independently selected from nitrogen, oxygen, and sulfur.

In some embodiments, Ring A is 5- to 6-membered monocyclic heteroaryl having 1-4 heteroatoms independently selected from nitrogen, oxygen, and sulfur. In some embodiments, Ring A is 5-membered monocyclic heteroaryl having 1-4 heteroatoms independently selected from nitrogen, oxygen, and sulfur. In some embodiments, Ring A is 5-membered monocyclic heteroaryl having 1-3 nitrogen atoms. In some embodiments, Ring A is 5-membered monocyclic heteroaryl having 1-2 nitrogen atoms. In some embodiments, Ring A is 6-membered monocyclic heteroaryl having 1-4 heteroatoms independently selected from nitrogen, oxygen, and sulfur. In some embodiments, Ring A is 6-membered monocyclic heteroaryl having 1-3 nitrogen atoms. In some embodiments, Ring A is 6-membered monocyclic heteroaryl having 1-2 nitrogen atoms. In some embodiments, Ring A is selected from pyrazolyl, imidazolyl, triazolyl, and pyridyl. In some embodiments, Ring A is pyrazolyl. In some embodiments, Ring A is imidazolyl. In some embodiments, Ring A is triazolyl. In some embodiments, Ring A is pyridyl.

As described above, in some embodiments of any of Formulae I, II, and III, each L^{A} is independently a covalent bond or optionally substituted bivalent C₁₋₆ aliphatic. In some embodiments of any of Formulae I, II, III, and IV, each L^{A} is independently a covalent bond or optionally substituted bivalent C₁₋₆ aliphatic. In some embodiments, L^{A} is a covalent bond or a bivalent C₁₋₂ aliphatic (e.g., —CH₂— or —CH₂CH₂—). In some embodiments, L^{A} is a covalent bond. In some embodiments, L^{A} is a bivalent C₁₋₆ aliphatic. In some embodiments, L^{A} is a bivalent C₁₋₃ aliphatic. In some embodiments, L^{A} is a bivalent C₁₋₂ aliphatic (e.g., —CH₂— or —CH₂CH₂—). In some embodiments, L^{A} is a bivalent C₁ aliphatic (e.g., —CH₂—). In some embodiments, L^{A} is a bivalent C₂ aliphatic (e.g., —CH₂CH₂—).

In some embodiments of any of Formulae I, II, III, and IV, each R^{A} is independently oxo, halogen, —CN, —OR^{A1}, —SR^{A1}, —N(R^{A1})₂, —C(O)R^{A1}, —C(O)OR^{A1}, —C(O)N(R^{A1})₂, —C(O)NR^{A1}(OR^{A1}), —OC(O)R^{A1}—OC(O)N(R^{A1})₂, —OC(O)OR^{A1}, —OSO₂R^{A1}, —OSO₂N(R^{A1})₂, —N(R^{A1})C(O)R^{A1}, —NR^{A1}C(O)OR^{A1}, —NR^{A1}C(O)N(R^{A1})₂, —N(R^{A1})SO₂R^{A1}, —NR^{A1}S(O)₂N(R^{A1})₂, —NR^{A1}OR^{A1}, —NR^{A1}S(O)R^{A1}, —NR^{A1}S(O)N(R^{A1})₂, —S(O)R^{A1}, —SO₂R^{A1}, —S(O)N(R^{A1})₂, —SO₂N(R^{A1})₂, —SO₃R^{A1}, —C(═NR^{m})R^{A1}, —C(═NR^{m})N(R^{A1})₂, —NR^{A1}C(═NR^{m})R^{A1}, —NR^{A1}C(═NR^{m})N(R^{A1})₂, —NR^{A1}S(O)(═NR^{m})R^{A1}, —NR^{A1}S(O)(═NR^{m}1)N(R^{A1})₂, —OS(O)(═NR^{m})R^{A1}, —S(O)(═NR^{m})R^{A1}, —S(O)(═NR^{m})N(R^{A1})₂, —P(O)(R^{A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents.

As described above, in some embodiments of any of Formulae I, II, and III, each R^{A} is independently oxo, halogen, —CN, —OR^{A1}, —SR^{A1}, —N(R^{A1})₂, —C(O)R^{A1}, —C(O)OR^{A1}, —C(O)N(R^{A1})₂, —C(O)NR^{A1}(OR^{A1}), —OC(O)R^{A1}—OC(O)N(R^{A1})₂, —OC(O)OR^{A1}, —OSO₂R^{A1}, —OSO₂N(R^{A1})₂, —N(R^{A1})C(O)R^{A1}, —NR^{A1}C(O)OR^{A1}, —NR^{A1}C(O)N(R^{A1})₂, —N(R^{A1})SO₂R^{A1}, —NR^{A1}S(O)₂N(R^{A1})₂, —NR^{A1}OR^{A1}, —NR^{A1}S(O)R^{A1}, —NR^{A1}S(O)N(R^{A1})₂, —S(O)R^{A1}, —SO₂R^{A1}, —S(O)N(R^{A1})₂, —SO₂N(R^{A1})₂, —SO₃R^{A1}, —C(═NR^{m})R^{A1}, —C(═NR^{m})N(R^{A1})₂, —NR^{A1}C(═NR^{m})R^{A1}, —NR^{A1}C(═NR^{m}1)N(R^{A1})₂, —NR^{A1}S(O)(═NR^{m})R^{A1}, —NR^{A1}S(O)(═NR^{m})N(R^{A1})₂, —OS(O)(═NR^{m})R^{A1}, —S(O)(═NR^{m})R^{A1}, —S(O)(═NR^{m}1)N(R^{A1})₂, —P(O)(R^{A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents.

In some embodiments, each R^{A} is independently C₁₋₆ aliphatic, —OR^{A1}, —N(R^{A1})₂, - to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents.

In some embodiments, each R^{A} is independently C₁₋₆ alkyl, C₂₋₆ alkenyl, —OR^{A1}, —N(R^{A1})₂, —C(O)R^{A1}, —C(O)N(R^{A1})₂, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur, wherein the C₁₋₆ alkyl, C₂₋₆ alkenyl, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl of R^{A} are each independently optionally substituted with 1, 2, 3, 4, 5, or 6 R^{AG} substituents.

In some embodiments, each R^{A} is independently C₁₋₆ alkyl, C₂₋₆ alkenyl, —OR^{A1}, —N(R^{A1})₂, —C(O)R^{A1}, —C(O)N(R^{A1})₂, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur, wherein the C₁₋₆ alkyl, C₂₋₆ alkenyl, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl of R^{A} are each independently optionally substituted with 1, 2, 3, or 4 R^{AG} substituents.

In some embodiments, each R^{A} is independently C₁₋₆ alkyl, C₂₋₆ alkenyl, —OR^{A1}, —N(R^{A1})₂, —C(O)R^{A1}, —C(O)N(R^{A1})₂, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur, wherein the C₁₋₆ alkyl, C₂₋₆ alkenyl, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl of R^{A} are each independently optionally substituted with 1 or 2 R^{AG} substituents.

In some embodiments, each R^{A} is independently C₁₋₆ alkyl, C₂₋₆ alkenyl, —OR^{A1}, —N(R^{A1})₂, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; wherein each of the C₁₋₆ alkyl, C₂₋₆ alkenyl, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents.

In some embodiments, each R^{A} is independently C₁₋₆ aliphatic, —OR^{A1}, —N(R^{A1})₂, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} are each optionally substituted with 1, 2, 3, 4, 5, or 6 independently selected R^{AG} substituents.

In some embodiments, each R^{A} is independently C₁₋₆ alkyl, C₂₋₆ alkenyl, —OR^{A1}, —N(R^{A1})₂, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; wherein each of the C₁₋₆ alkyl, C₂₋₆ alkenyl, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} are each optionally substituted with 1, 2, 3, or 4 independently selected R^{AG} substituents.

In some embodiments, each R^{A} is independently C₁₋₆ alkyl, C₂₋₆ alkenyl, —OR^{A1}, —N(R^{A1})₂, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; wherein each of the C₁₋₆ alkyl, C₂₋₆ alkenyl, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} are each optionally substituted with 1 or 2 independently selected R^{AG} substituents.

In some embodiments, each R^{A} is independently C₁₋₆ alkyl, C₂₋₆ alkenyl, —OR^{A1}, —N(R^{A1})₂, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur, wherein the C₁₋₆ alkyl, C₂₋₆ alkenyl, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl of R^{A} are each independently optionally substituted with 1 or 2 R^{AG} substituents.

In some embodiments, each R^{A} is independently C₁₋₆ alkyl, C₂₋₆ alkenyl, —OR^{A1}, —N(R^{A1})₂, phenyl, 3- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur, wherein the C₁₋₆ alkyl, C₂₋₆ alkenyl, phenyl, 3- to 7-membered saturated or partially unsaturated monocyclic heterocyclyl, and 5- to 6-membered monocyclic heteroaryl of R^{A} are each independently optionally substituted with 1 or 2 R^{AG} substituents.

In some embodiments, each R^{A} is independently C₁₋₆ alkyl, C₂₋₆ alkenyl, —OR^{A1}, —N(R^{A1})₂, phenyl, 3- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur, wherein the C₁₋₆ alkyl, C₂₋₆ alkenyl, phenyl, 3- to 7-membered saturated or partially unsaturated monocyclic heterocyclyl, and 5- to 6-membered monocyclic heteroaryl of R^{A} are each independently optionally substituted with 1 or 2 R^{AG} substituents.

In some embodiments, each R^{A} is independently selected from phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, each of which is optionally substituted with 1, 2, 3, 4, 5, or 6 independently selected R^{AG} substituents.

In some embodiments, each R^{A} is independently selected from phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, each of which is optionally substituted with 1 or 2 independently selected R^{AG} substituents.

In some embodiments, each R^{A} is independently selected from 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, each of which is optionally substituted with 1 or 2 independently selected R^{AG} substituents.

In some embodiments, each R^{A} is independently selected from 3- to 7-membered saturated or partially unsaturated monocyclic heterocyclyl having 1-3 heteroatoms independently selected from nitrogen, oxygen, and sulfur, each of which is optionally substituted with 1 or 2 independently selected R^{AG} substituents.

In some embodiments, each R^{A} is independently selected from 5- to 6-membered monocyclic heteroaryl having 1-4 heteroatoms independently selected from nitrogen, oxygen, and sulfur, each of which is optionally substituted with 1 or 2 independently selected R^{AG} substituents.

In some embodiments, R^{A} is phenyl optionally substituted with 1, 2, 3, 4, 5, or 6 R^{AG} substituents. In some embodiments, R^{A} is phenyl.

In some embodiments, R^{A} is C₁₋₆ aliphatic optionally substituted with 1, 2, 3, 4, 5, or 6 R^{AG} substituents. In some embodiments, R^{A} is C₁₋₃ aliphatic optionally substituted with 1, 2, 3, 4, 5, or 6 R^{AG} substituents. In some embodiments, R^{A} is C₁₋₂ aliphatic optionally substituted with 1, 2, 3, 4, or 5 R^{AG} substituents. In some embodiments, R^{A} is C₁₋₂ aliphatic optionally substituted with 1, 2, 3, 4, or 5 R^{AG} substituents. In some embodiments, R^{A} is C₂ alkenyl optionally substituted with 1, 2, or 3 R^{AG} substituents. In some embodiments, R^{A} is —CH═CH₂.

In some embodiments, two L^{A}-R^{A} taken together with the atoms to which they are attached form a 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl is independently optionally substituted with 1, 2, 3, 4, 5, or 6 R^{AG} substituents.

In some embodiments, each R^{A1} is independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A1} is independently optionally substituted with 1, 2, 3, 4, 5, or 6 R^{G1} substituents; or two R^{A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

In some embodiments, each R^{A1} is independently C₁₋₆ aliphatic or 3- to 7-membered saturated or partially unsaturated monocyclic heterocyclyl having 1-3 heteroatoms independently selected from nitrogen, oxygen, and sulfur, wherein each C₁₋₆ aliphatic and 3- to 7-membered saturated or partially unsaturated monocyclic heterocyclyl of R^{A1} is optionally substituted with 1, 2, 3, 4, 5, or 6 independently selected R^{G1} substituents.

In some embodiments, each R^{A1} is independently C₁₋₆ aliphatic or 3- to 7-membered saturated or partially unsaturated monocyclic heterocyclyl having 1-3 heteroatoms independently selected from nitrogen, oxygen, and sulfur, wherein each C₁₋₆ aliphatic and 3- to 7-membered saturated or partially unsaturated monocyclic heterocyclyl of R^{A1} is optionally substituted with 1 or 2 independently selected R^{G1} substituents.

In some embodiments, each R^{A1} is independently C₁₋₆ alkyl or 3- to 7-membered saturated or partially unsaturated monocyclic heterocyclyl having 1-3 heteroatoms independently selected from nitrogen, oxygen, and sulfur, wherein each C₁₋₆ aliphatic and 3- to 7-membered saturated or partially unsaturated monocyclic heterocyclyl of R^{A1} is optionally substituted with 1, 2, 3, 4, 5, or 6 independently selected R^{G1} substituents.

In some embodiments, each R^{A1} is independently C₁₋₆ alkyl or 3- to 7-membered saturated or partially unsaturated monocyclic heterocyclyl having 1-3 heteroatoms independently selected from nitrogen, oxygen, and sulfur, wherein each C₁₋₆ aliphatic and 3- to 7-membered saturated or partially unsaturated monocyclic heterocyclyl of R^{A1} is optionally substituted with 1 or 2 independently selected R^{G1} substituents.

In some embodiments, each R^{A1} is independently C₁₋₆ aliphatic, wherein each C₁₋₆ aliphatic of R^{A1} is optionally substituted with 1, 2, 3, 4, 5, or 6 independently selected R^{G1} substituents.

In some embodiments, each R^{A1} is independently C₁₋₆ alkyl, wherein each C₁₋₆ alkyl of R^{A1} is optionally substituted with 1, 2, 3, 4, 5, or 6 independently selected R^{G1} substituents. In some embodiments, each R^{A1} is independently C₁₋₆ alkyl, wherein each C₁₋₆ alkyl of R^{A1} is optionally substituted with 1, 2, 3, or 4 independently selected R^{G1} substituents. In some embodiments, each R^{A1} is independently C₁₋₆ alkyl, wherein each C₁₋₆ alkyl of R^{A1} is optionally substituted with 1 or 2 independently selected R^{G1} substituents.

In some embodiments, each R^{G1} is independently selected from OR, where each R is hydrogen or C₁₋₆ aliphatic. In some embodiments, each R^{G1} is independently selected from OR, where each R is hydrogen or C₁₋₆ alkyl. In some embodiments, each R^{G1} is independently selected from OR, where each R is C₁₋₆ alkyl. In some embodiments, each R^{G1} is independently selected from OR, where each R is C₁₋₃ alkyl. In some embodiments, each R^{G1} is hydroxy or methoxy. In some embodiments, each R^{G1} is methoxy. In some embodiments, each R^{G1} is hydroxy.

In some embodiments, each R^{A1} is independently C₁₋₆ aliphatic or 3- to 7-membered saturated or partially unsaturated monocyclic heterocyclyl having 1-3 heteroatoms independently selected from nitrogen, oxygen, and sulfur, wherein each C₁₋₆ alkyl and 3- to 7-membered saturated or partially unsaturated monocyclic heterocyclyl of R^{A1} is optionally substituted with hydroxy or methoxy.

In some embodiments, each R^{A1} is independently C₁₋₆ alkyl or 3- to 7-membered saturated or partially unsaturated monocyclic heterocyclyl having 1-3 heteroatoms independently selected from nitrogen, oxygen, and sulfur, wherein each C₁₋₆ alkyl and 3- to 7-membered saturated or partially unsaturated monocyclic heterocyclyl of R^{A1} is optionally substituted with hydroxy or methoxy.

In some embodiments, each R^{A1} is independently C₁₋₆ alkyl, wherein each C₁₋₆ alkyl of R^{A1} is optionally substituted with methoxy.

In some embodiments, each R^{AG} is independently halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments, each R^{AG} is independently selected from halogen and C₁₋₆ aliphatic. In some embodiments, each R^{AG} is an independently selected halogen. In some embodiments, each R^{AG} is an independently selected C₁₋₆ aliphatic. In some embodiments, each R^{AG} is an independently selected C₁₋₆ alkyl. In some embodiments, each R^{AG} is an independently selected C₁₋₃ alkyl. In some embodiments, each R^{AG} is independently selected from fluoro and methyl. In some embodiments, each R^{AG} is fluoro. In some embodiments, each R^{AG} is methyl.

In some embodiments, each R^{A} is independently selected from methyl, ethyl, n-propyl, isopropyl, etheneyl, methoxy, methoxyethoxy, dimethylamino, dimethylaminocarbonyl, azetidinylcarbonyl, (hydroxyazetidinyl)carbonyl, cyclopropyl, cyclopentyl, phenyl, pyridyl, pyrimidinyl, tetrahydrofuranyl, tetrahydropyranyl, azetidinyl, pyrrolidinyl, piperidinyl, tetrahydro-2H-pyranyl, morpholinyl, tetrahydrofuranyl, piperazinyl, 5,6-dihydro-[1,2,4]triazolo[1,5-a]pyrazin-7(8H)-yl, wherein the methyl, ethyl, n-propyl, isopropyl, etheneyl, cyclopropyl, cyclopentyl, phenyl, pyridyl, pyrimidinyl, tetrahydrofuranyl, tetrahydropyranyl, azetidinyl, pyrrolidinyl, piperidinyl, tetrahydro-2H-pyranyl, morpholinyl, tetrahydrofuranyl, piperazinyl, and 5,6-dihydro-[1,2,4]triazolo[1,5-a]pyrazin-7(8H)-yl are each optionally substituted with 1, 2, 3, or 4 independently selected R^{AG} substituents.

In some embodiments, each R^{A} is independently selected from methyl, ethyl, n-propyl, isopropyl, etheneyl, methoxy, methoxyethoxy, dimethylamino, dimethylaminocarbonyl, azetidinylcarbonyl, (hydroxyazetidinyl)carbonyl, cyclopropyl, cyclopentyl, phenyl, pyridyl, pyrimidinyl, tetrahydrofuranyl, tetrahydropyranyl, azetidinyl, pyrrolidinyl, piperidinyl, tetrahydro-2H-pyranyl, morpholinyl, tetrahydrofuranyl, piperazinyl, 5,6-dihydro-[1,2,4]triazolo[1,5-a]pyrazin-7(8H)-yl, wherein the methyl, ethyl, n-propyl, isopropyl, etheneyl, cyclopropyl, cyclopentyl, phenyl, pyridyl, pyrimidinyl, tetrahydrofuranyl, tetrahydropyranyl, azetidinyl, pyrrolidinyl, piperidinyl, tetrahydro-2H-pyranyl, morpholinyl, tetrahydrofuranyl, piperazinyl, and 5,6-dihydro-[1,2,4]triazolo[1,5-a]pyrazin-7(8H)-yl are each optionally substituted with 1 or 2 independently selected R^{AG} substituents.

In some embodiments, each R^{A} is independently selected from methyl, ethyl, n-propyl, isopropyl, etheneyl, methoxy, methoxyethoxy, dimethylamino, cyclopropyl, cyclopentyl, phenyl, methylphenyl, pyridyl, fluoropyridyl, methylpyridyl, pyrimidinyl, tetrahydrofuranyl, and tetrahydropyranyl.

In some embodiments, each R^{A} is independently selected from methyl, ethyl, propyl, etheneyl, methoxy, methoxyethoxy, dimethylamino, phenyl, methylphenyl, pyridyl, fluoropyridyl, methylpyridyl, pyrimidinyl, and tetrahydropyranyl.

In some embodiments, each L^{A} is a covalent bond or a C₁₋₃ aliphatic. In some embodiments, each L^{A} is a covalent bond or a C₁₋₂ aliphatic. In some embodiments, each L^{A} is a covalent bond. In some embodiments, each L^{A} is a C₁₋₃ aliphatic. In some embodiments, each L^{A} is a C₁₋₂ aliphatic.

In some embodiments, each L^{A} is a covalent bond or a C₁₋₂ aliphatic, and each R^{A} is independently selected from methyl, ethyl, n-propyl, isopropyl, etheneyl, methoxy, methoxyethoxy, dimethylamino, dimethylaminocarbonyl, azetidinylcarbonyl, (hydroxyazetidinyl)carbonyl, cyclopropyl, cyclopentyl, phenyl, pyridyl, pyrimidinyl, tetrahydrofuranyl, tetrahydropyranyl, azetidinyl, pyrrolidinyl, piperidinyl, tetrahydro-2H-pyranyl, morpholinyl, tetrahydrofuranyl, piperazinyl, 5,6-dihydro-[1,2,4]triazolo[1,5-a]pyrazin-7(8H)-yl, wherein the methyl, ethyl, n-propyl, isopropyl, etheneyl, cyclopropyl, cyclopentyl, phenyl, pyridyl, pyrimidinyl, tetrahydrofuranyl, tetrahydropyranyl, azetidinyl, pyrrolidinyl, piperidinyl, tetrahydro-2H-pyranyl, morpholinyl, tetrahydrofuranyl, piperazinyl, and 5,6-dihydro-[1,2,4]triazolo[1,5-a]pyrazin-7(8H)-yl are each optionally substituted with 1 or 2 independently selected R^{AG} substituents.

In some embodiments, each L^{A} is a covalent bond or a C₂ aliphatic, and each R^{A} is independently selected from methyl, ethyl, propyl, etheneyl, methoxy, methoxyethoxy, dimethylamino, phenyl, methylphenyl, pyridyl, fluoropyridyl, methylpyridyl, pyrimidinyl, and tetrahydropyranyl.

In some embodiments, each L^{A} is a covalent bond and each R^{A} is independently selected from methyl, ethyl, propyl, etheneyl, methoxy, methoxyethoxy, dimethylamino, phenyl, methylphenyl, pyridyl, fluoropyridyl, methylpyridyl, pyrimidinyl, and tetrahydropyranyl.

In some embodiments, each L^{A} is a C₂ aliphatic and each R^{A} is independently selected from methyl, ethyl, propyl, etheneyl, methoxy, methoxyethoxy, dimethylamino, phenyl, methylphenyl, pyridyl, fluoropyridyl, methylpyridyl, pyrimidinyl, and tetrahydropyranyl. In some embodiments, each L^{A} is a C₂ aliphatic and each R^{A} is phenyl.

In some embodiments,

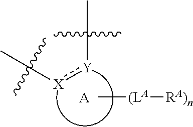

is

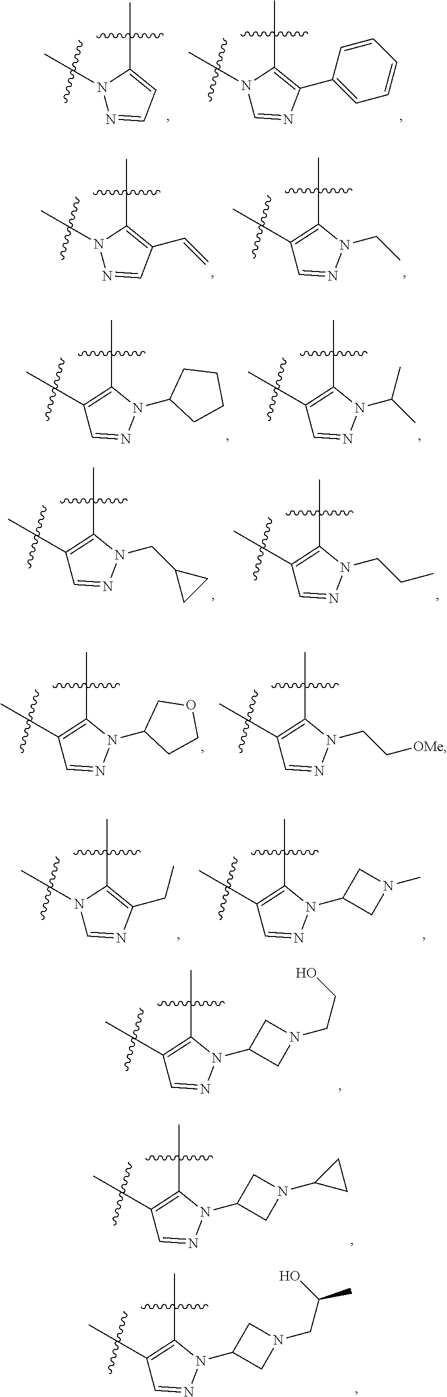

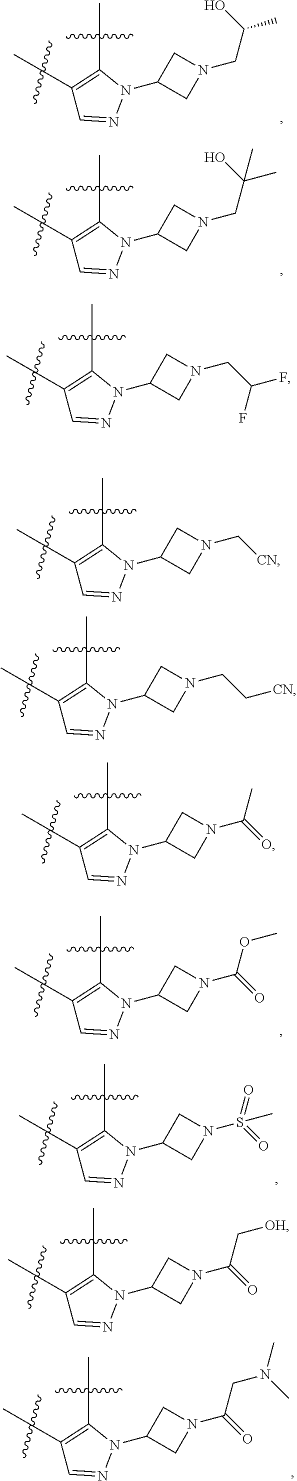

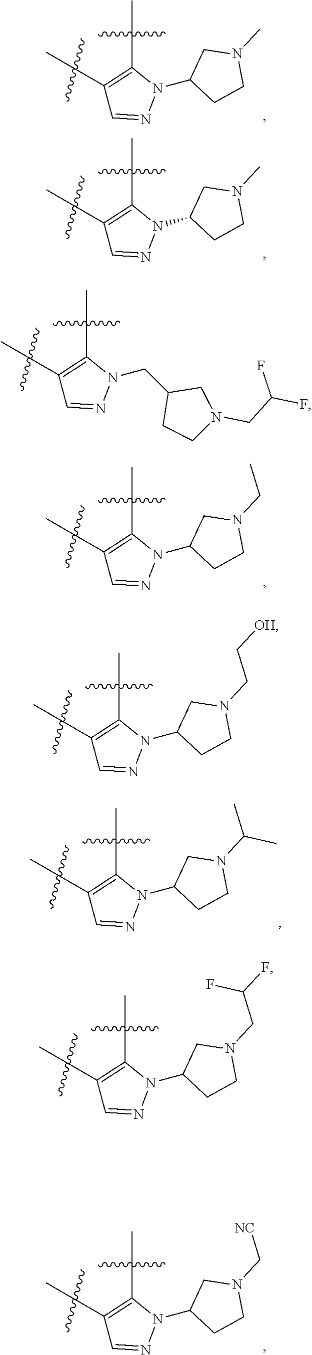

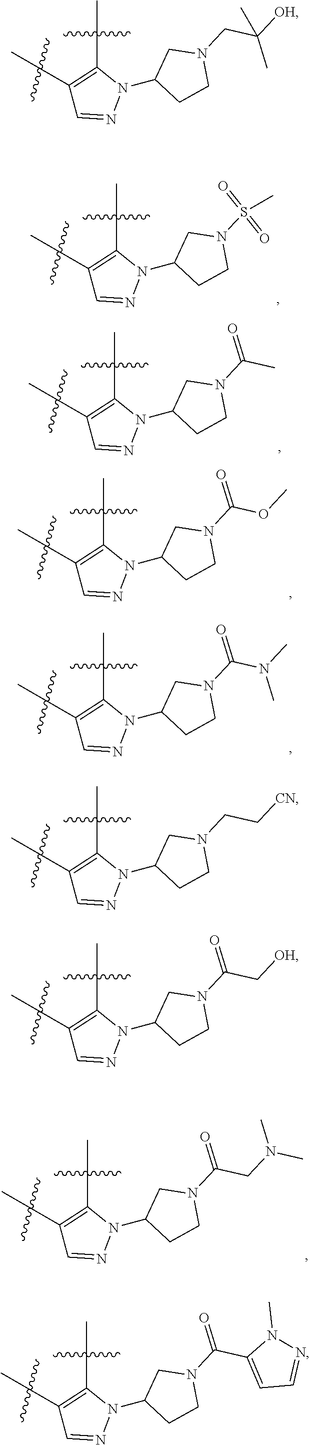

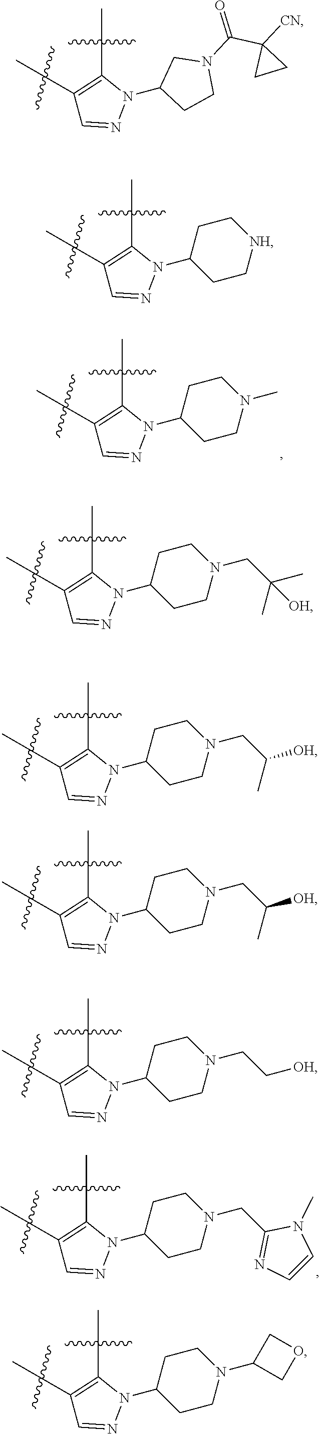

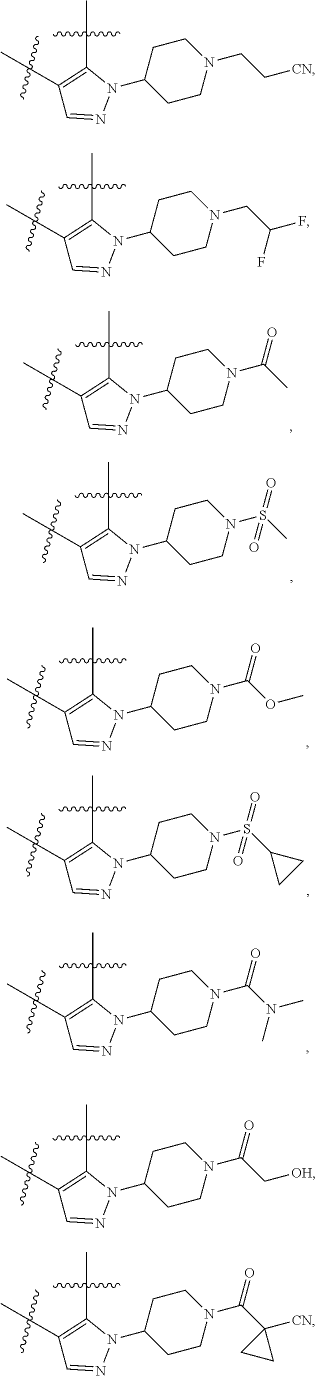

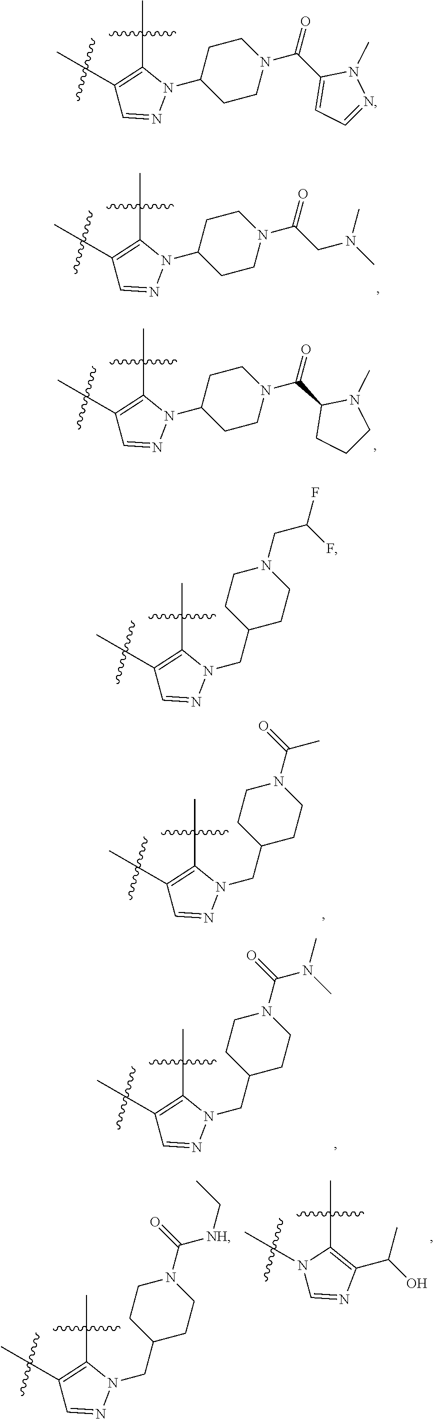

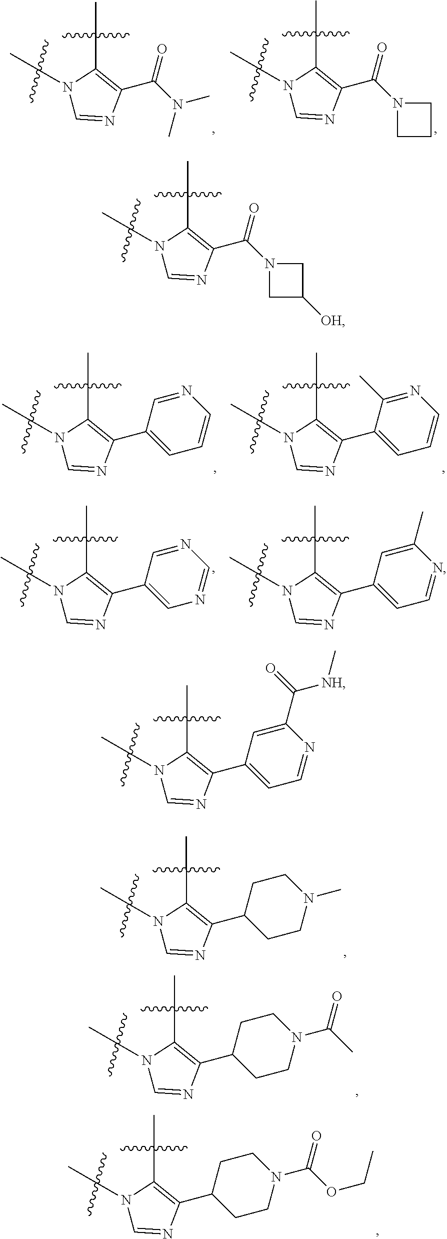

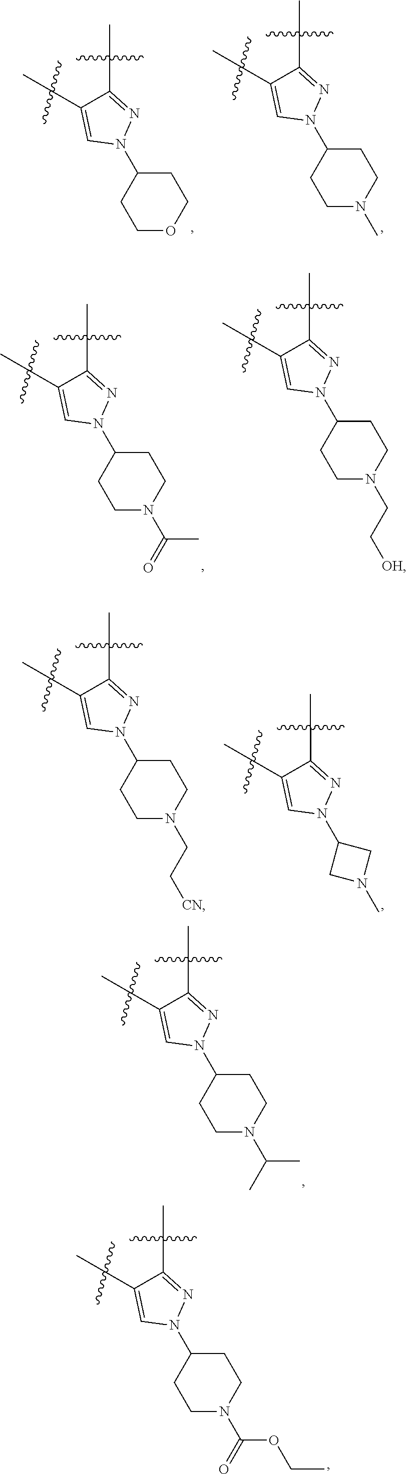

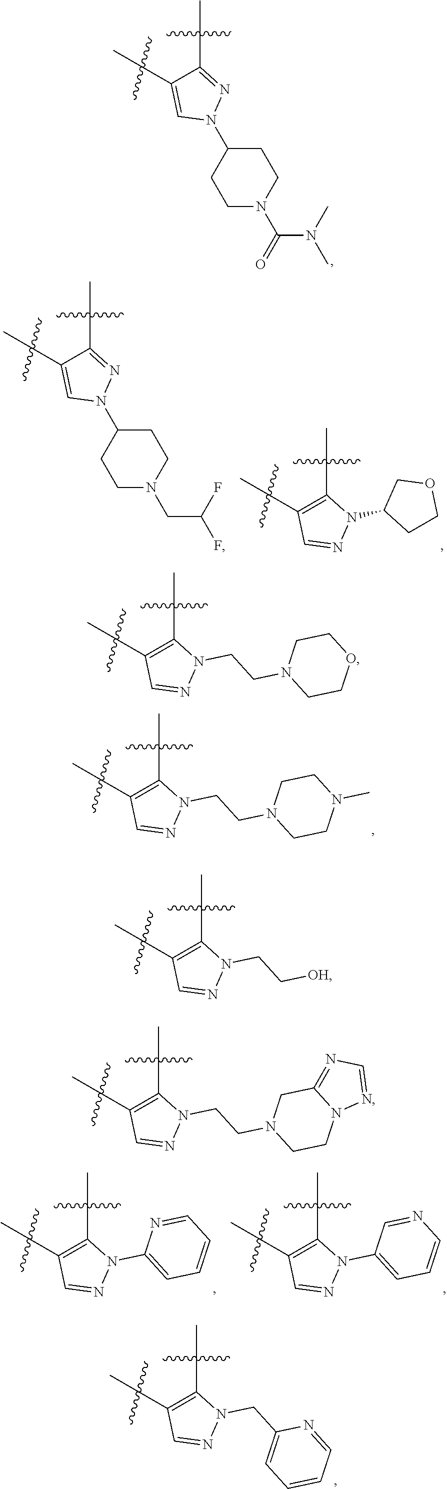

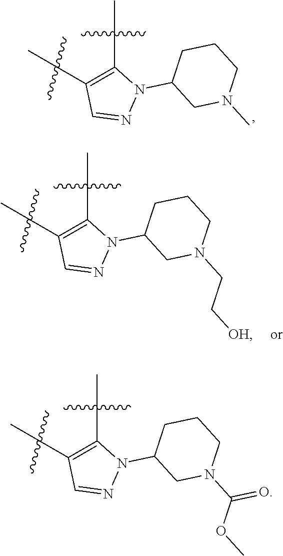 [SMILES: *c1cnn(C2CCCN(C)C2)c1* ; *c1cnn(C2CCCN(CCO)C2)c1* ; *c1cnn(C2CCCN(C(=O)OC)C2)c1*]

In some embodiments,

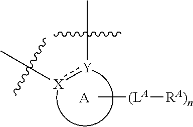

is

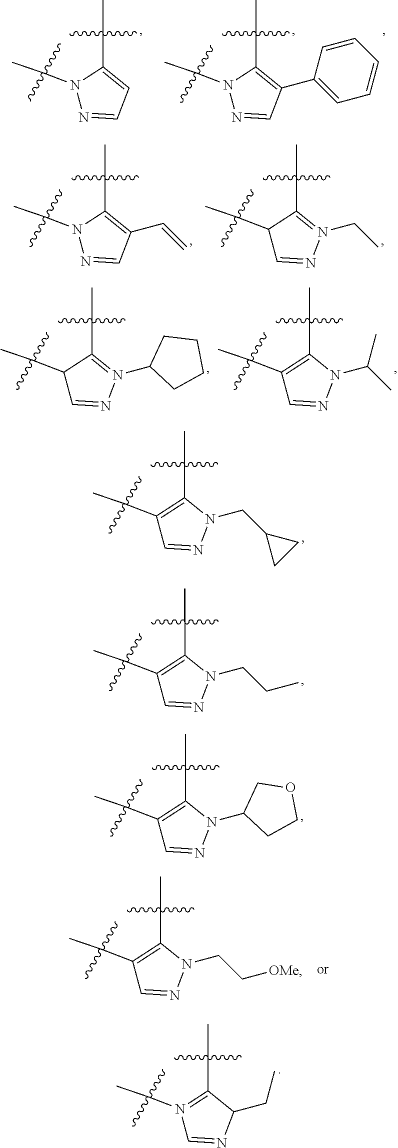

In some embodiments,

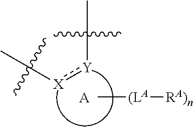

is

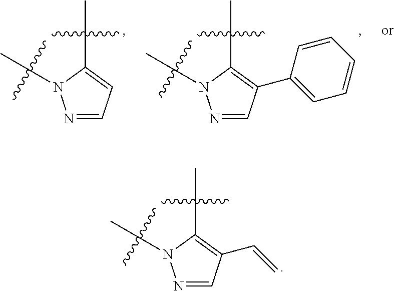

In some embodiments of any of Formulae I, II, III, IV, VI, and VIa, R¹ is hydrogen or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur.

As described above, in some embodiments of any of Formulae I, II, and III, R¹ is hydrogen or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur.

In some embodiments, R¹ is an optionally substituted C₁₋₆ aliphatic, 3- to 7-membered saturated or partially unsaturated monocyclic carbocyclyl, or 3- to 7-membered saturated or partially unsaturated monocyclic heterocyclyl having 1-3 heteroatoms independently selected from nitrogen, oxygen, and sulfur.

In some embodiments, R¹ is an optionally substituted C₁₋₆ alkyl, 3- to 7-membered saturated or partially unsaturated monocyclic carbocyclyl, or 3- to 7-membered saturated or partially unsaturated monocyclic heterocyclyl having 1-3 heteroatoms independently selected from nitrogen, oxygen, and sulfur.

In some embodiments, R¹ is a 3- to 7-membered saturated or partially unsaturated monocyclic carbocyclyl. In some embodiments, R¹ is cyclopropyl.

In some embodiments, R¹ is a 3- to 7-membered saturated or partially unsaturated monocyclic heterocyclyl having 1-3 heteroatoms independently selected from nitrogen, oxygen, and sulfur. In some embodiments, R¹ is tetrahydrofuranyl.

In some embodiments, R¹ is optionally substituted C₁₋₆ aliphatic. In some embodiments, R¹ is optionally substituted C₁₋₃ aliphatic. In some embodiments, R¹ is optionally substituted C₁₋₂ aliphatic.

In some embodiments, R¹ is C₁-6 alkyl. In some embodiments, R¹ is C₁-3 alkyl. In some embodiments, R¹ is C₁-2 alkyl. In some embodiments, R¹ is optionally substituted C₁₋₂ alkyl. In some embodiments, R¹ is C₁-2 alkyl, which is optionally substituted with 1, 2, 3, or 4 halogen groups. In some embodiments, R¹ is optionally substituted C₁₋₂ alkyl. In some embodiments, R¹ is C₁-2 alkyl, which is optionally substituted with 1 or 2 halogen groups. In some embodiments, R¹ is optionally substituted C₁ aliphatic. In some embodiments, R¹ is methyl, ethyl, or difluoroethyl. In some embodiments, R¹ is methyl or ethyl. In some embodiments, R¹ is methyl. In some embodiments, R¹ is ethyl. In some embodiments, R¹ is difluoroethyl.

In some embodiments, R¹ is methyl, trideuteromethyl, ethyl, difluoroethyl, hydroxyethyl, cyclopropyl, or tetrahydrofuranyl.

In some embodiments, R¹ and one of L^{A}-R^{A} taken together with the atoms to which they are attached form a 3- to 7-membered saturated or partially unsaturated monocyclic heterocyclyl having 1-3 heteroatoms independently selected from nitrogen, oxygen, and sulfur, or 5- to 14-membered saturated or partially unsaturated bicyclic heterocyclyl having 1-4 heteroatoms independently selected from nitrogen, oxygen, and sulfur, wherein each of the 3- to 7-membered monocyclic heterocyclyl and 5- to 14-membered bicyclic heterocyclyl is independently optionally substituted with 1, 2, 3, 4, 5, or 6 R^{AG} substituents.

In some embodiments, R¹ and one of L^{A}-R^{A} taken together with the atoms to which they are attached form a 3- to 7-membered saturated or partially unsaturated monocyclic heterocyclyl having 1-3 heteroatoms independently selected from nitrogen, oxygen, and sulfur, which is optionally substituted with 1, 2, 3, 4, 5, or 6 R^{AG} substituents.

In some embodiments, R¹ and one of L^{A}-R^{A} taken together with the atoms to which they are attached form a 3- to 7-membered saturated or partially unsaturated monocyclic heterocyclyl having 1-3 heteroatoms independently selected from nitrogen, oxygen, and sulfur, which is optionally substituted with 1, 2, 3, or 4 R^{AG} substituents.

In some embodiments, R¹ and one of L^{A}-R^{A} taken together with the atoms to which they are attached form a 3- to 7-membered saturated or partially unsaturated monocyclic heterocyclyl having 1-3 heteroatoms independently selected from nitrogen, oxygen, and sulfur, which is optionally substituted with 1 or 2 R^{AG} substituents.

In some embodiments, R¹ and one of L^{A}-R^{A} taken together with the atoms to which they are attached form a 6-membered saturated or partially unsaturated monocyclic heterocyclyl having 1-3 heteroatoms independently selected from nitrogen, oxygen, and sulfur, which is optionally substituted with 1, 2, 3, 4, 5, or 6 R^{AG} substituents.

In some embodiments, R¹ and one of L^{A}-R^{A} taken together with the atoms to which they are attached form a 6-membered saturated or partially unsaturated monocyclic heterocyclyl having 1-3 heteroatoms independently selected from nitrogen, oxygen, and sulfur, which is optionally substituted with 1, 2, 3, or 4 R^{AG} substituents.

In some embodiments, R¹ and one of L^{A}-R^{A} taken together with the atoms to which they are attached form a 6-membered saturated or partially unsaturated monocyclic heterocyclyl having 1-3 heteroatoms independently selected from nitrogen, oxygen, and sulfur, which is optionally substituted with 1 or 2 R^{AG} substituents.

In some embodiments, R¹ and one of L^{A}-R^{A} taken together with the atoms to which they are attached form a 6-membered saturated or partially unsaturated monocyclic heterocyclyl having 1-3 heteroatoms independently selected from nitrogen, oxygen, and sulfur, which is optionally substituted with 1 or 2 R^{AG} substituents.

In some embodiments, the compound of Formula I is a compound of Formula IV:

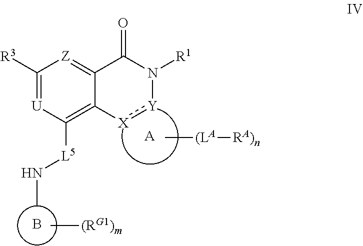

or a pharmaceutically acceptable salt thereof.

In some embodiments of Formula IV:
L⁵ is a bivalent C₁₋₃ aliphatic which is optionally substituted with 1, 2, 3, or 4 independently selected R^{L5A} substituents;
or L⁵ and one of L^{A}-R^{A} taken together with the atoms to which they are attached form a 3- to 16-membered saturated or partially unsaturated monocyclic carbocyclyl, 5- to 16-membered saturated or partially unsaturated bicyclic carbocyclyl, 3- to 16-membered saturated or partially unsaturated monocyclic heterocyclyl having 1-3 heteroatoms independently selected from nitrogen, oxygen, and sulfur, or 5- to 16-membered saturated or partially unsaturated bicyclic heterocyclyl having 1-4 heteroatoms independently selected from nitrogen, oxygen, and sulfur, wherein each of the 3- to 16-membered monocyclic carbocyclyl, 5- to 16-membered bicyclic carbocyclyl, 3- to 16-membered monocyclic heterocyclyl, and 5- to 16-membered bicyclic heterocyclyl is independently optionally substituted with 1, 2, 3, 4, 5, or 6 R^{AG} substituents;
Ring B is 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; and
m is 0, 1, 2, 3, 4, 5, or 6.

In some embodiments of Formula IV:
L⁵ is a bivalent C₁₋₃ aliphatic which is optionally substituted with 1, 2, 3, or 4 independently selected R^{L5A} substituents;
or L⁵ and one of L^{A}-R^{A} taken together with the atoms to which they are attached form a 3- to 7-membered saturated or partially unsaturated monocyclic carbocyclyl, 5- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, or 5- to 10-membered saturated or partially unsaturated bicyclic heterocyclyl having 1-4 heteroatoms independently selected from nitrogen, oxygen, and sulfur, wherein each of the 3- to 7-membered monocyclic carbocyclyl, 5- to 10-membered bicyclic carbocyclyl, 3- to 7-membered monocyclic heterocyclyl, and 5- to 10-membered bicyclic heterocyclyl is independently optionally substituted with 1, 2, 3, 4, 5, or 6 R^{AG} substituents;
Ring B is 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; and
m is 0, 1, 2, 3, 4, 5, or 6.

In some embodiments of any of Formulae I, II, III, IV, V, Va, VI, VIa, VII, VIIa, and VIIb, Z is N or CR². In some embodiments of any of Formulae I, II, III, IV, V, Va, VI, and VIa, Z is N or CR². As described above, in some embodiments of any of Formulae I, II, and III, Z is N or CR². In some embodiments, Z is CR². In some embodiments, Z is CH.

In some embodiments, R² is hydrogen, halogen, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur.

In some embodiments, R² is hydrogen.

In some embodiments of any of Formulae I, II, III, IV, V, Va, VI, VIa, VII, VIIa, and VIIb, R³ is —F, —Cl, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m}1)R, —NRC(═NR^{m})N(R)₂, —NRS(O)(═NR^{m})R, —NRS(O)(═NR^{m})N(R)₂, —OS(O)(═NR¹)R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur.

In some embodiments of any of Formulae I, II, III, IV, V, Va, VI, and VIa, R³ is —F, —Cl, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur.

As described above, in some embodiments of any of Formulae I, II, and III, R³ is —F, —Cl, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur.

In some embodiments, R³ is —F, —Cl, —CN, or optionally substituted C₁₋₆ aliphatic. In some embodiments, R³ is —F, —Cl, or —CN. In some embodiments, R³ is —F or —Cl. In some embodiments, R³ is optionally substituted C₁₋₆ aliphatic. In some embodiments, R³ is optionally substituted C₁₋₆ aliphatic or —Cl. In some embodiments, R³ is optionally substituted C₁₋₃ aliphatic. In some embodiments, R³ is optionally substituted C₁₋₃ aliphatic or —Cl. In some embodiments, R³ is optionally substituted C₁₋₂ aliphatic. In some embodiments, R³ is optionally substituted C₁₋₂ aliphatic or —Cl. In some embodiments, R³ is optionally substituted C₁ aliphatic. In some embodiments, R³ is methyl, —CF3, or —CF2H. In some embodiments, R³ is methyl. In some embodiments, R³ is —Cl. In some embodiments, R³ is methyl or —Cl. In some embodiments, R³ is methyl or —CF3. In some embodiments, R³ is —CF3 (i.e., trifluoromethyl).

In some embodiments of any of Formulae I, II, III, IV, V, Va, VI, VIa, VII, VIIb, and VIIb, U is N or CR⁴. In some embodiments of any of Formulae I, II, III, IV, V, Va, VI, and VIa, U is N or CR⁴. As described above, in some embodiments of any of Formulae I, II, and III, U is N or CR⁴. In some embodiments, U is CR⁴. In some embodiments, U is CH.

In some embodiments, R⁴ is hydrogen, halogen, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur.

In some embodiments, R⁴ is hydrogen.

As described above, in some embodiments of Formula I, R⁵ is -L⁵-R^{5A}.

As described above, in some embodiments of any of Formula I, II, and III, L⁵ is a covalent bond or optionally substituted bivalent C₁₋₆ aliphatic. In some embodiments, L⁵ is optionally substituted bivalent C₁₋₆ aliphatic. In some embodiments, L⁵ is a bivalent C₁₋₆ aliphatic, which is optionally substituted by hydroxy. In some embodiments, L⁵ is optionally substituted bivalent C₁₋₃ aliphatic. In some embodiments, L⁵ is a bivalent C₁₋₃ aliphatic, which is optionally substituted by hydroxy. In some embodiments, L⁵ is optionally substituted bivalent C₁₋₂ aliphatic. In some embodiments, L⁵ is bivalent C₁₋₂ aliphatic, which is optionally substituted by hydroxy.

In some embodiments, L⁵ is bivalent C₁₋₂ aliphatic, which is optionally substituted by hydroxy or 5-6 membered saturated or partially unsaturated monocyclic heterocyclyl having 1-3 heteroatoms independently selected from nitrogen, oxygen, and sulfur, and wherein the 5-6 membered saturated or partially unsaturated monocyclic heterocyclyl is optionally substituted by C₁₋₆ alkyl.

In some embodiments, L⁵ is bivalent C₁₋₂ aliphatic, which is optionally substituted by hydroxy or 5-6 membered saturated or partially unsaturated monocyclic heterocyclyl having 1-3 heteroatoms independently selected from nitrogen, oxygen, and sulfur, and wherein the 5-6 membered saturated or partially unsaturated monocyclic heterocyclyl is optionally substituted by C₁₋₃ alkyl.

In some embodiments, L⁵ is bivalent C₁₋₂ aliphatic, which is optionally substituted by hydroxy or piperazinyl, wherein the piperazinyl is optionally substituted by C₁₋₆ alkyl.

In some embodiments, L⁵ is bivalent C₁₋₂ aliphatic, which is optionally substituted by hydroxy or piperazinyl, wherein the piperazinyl is optionally substituted by C₁₋₃ alkyl.

In some embodiments, L⁵ is bivalent C₁₋₂ aliphatic, which is optionally substituted by hydroxy or methylpiperazinyl.

In some embodiments, L⁵ is —CH(CH₃)—. In some embodiments, L⁵ is —CH(CH₃)— or —CH(CH₂OH)—. In some embodiments, L⁵ is —CH(CH₂OH)—. In some embodiments, L⁵ is —CH₂—, —CH(CH₃)—, —CH(CH₂OH)—, or

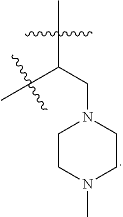 [SMILES: *C(*)CN1CCN(C)CC1]

In some embodiments, L⁵ is

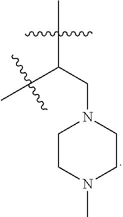 [SMILES: *C(*)CN1CCN(C)CC1]

In some embodiments, L⁵ and one of L^{A}-R^{A} taken together with the atoms to which they are attached form a 3- to 16-membered saturated or partially unsaturated monocyclic carbocyclyl, 5- to 16-membered saturated or partially unsaturated bicyclic carbocyclyl, 3- to 16-membered saturated or partially unsaturated monocyclic heterocyclyl having 1-3 heteroatoms independently selected from nitrogen, oxygen, and sulfur, or 5- to 16-membered saturated or partially unsaturated bicyclic heterocyclyl having 1-4 heteroatoms independently selected from nitrogen, oxygen, and sulfur, wherein each of the 3- to 16-membered monocyclic carbocyclyl, 5- to 16-membered bicyclic carbocyclyl, 3- to 16-membered monocyclic heterocyclyl, and 5- to 16-membered bicyclic heterocyclyl is independently optionally substituted with 1, 2, 3, 4, 5, or 6 R^{AG} substituents.

In some embodiments, L⁵ and one of L^{A}-R^{A} taken together with the atoms to which they are attached form a 3- to 7-membered saturated or partially unsaturated monocyclic carbocyclyl, 5- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, or 5- to 10-membered saturated or partially unsaturated bicyclic heterocyclyl having 1-4 heteroatoms independently selected from nitrogen, oxygen, and sulfur, wherein each of the 3- to 7-membered monocyclic carbocyclyl, 5- to 10-membered bicyclic carbocyclyl, 3- to 7-membered monocyclic heterocyclyl, and 5- to 10-membered bicyclic heterocyclyl is independently optionally substituted with 1, 2, 3, 4, 5, or 6 R^{AG} substituents.

As described above, in some embodiments of Formula I, R^{5A} is —OR^{5A11}, —SR^{5A1}, —NHR^{5A12}, —N(R^{5A12})₂, —C(O)R^{5A1}, —C(O)N(R^{5A1})₂, —C(O)NR^{5A1} (OR^{5A1}), —OC(O)R^{5A1}, —OC(O)N(R^{5A1})₂, —OC(O)OR^{5A1}, —OSO₂R^{5A1}, —OSO₂N(R^{5A1})₂, —N(R^{5A1})C(O)R^{5A1}, —NR^{5A1}C(O)OR^{5A1}, —NR^{5A1}C(O)N(R^{5A1})₂, —N(R^{5A1})SO₂R^{5A1}, —NR^{5A1}S(O)₂N(R^{5A1})₂, —NR^{5A1}OR^{5A1}, —NR^{5A1}S(O)R^{5A1}, —NR^{5A1}S(O)N(R^{5A1})₂, —S(O)R^{5A1}, —SO₂R^{5A1}, —S(O)N(R^{5A1})₂, —SO₂N(R^{5A1})₂, —SO₃R^{5A1}, —C(═NR^{m})R^{5A1}, —C(═NR^{m})N(R^{5A1})₂, —NR^{5A1}C(═NR^{m})R^{5A1}, —NR^{5A1}C(═NR^{m})N(R^{5A1})₂, —NR^{5A1}S(O)(═NR^{m})R^{5A1}, —NR^{5A1}S(O)(═NR^{m})N(R^{5A1})₂, —OS(O)(═NR^{m})R^{5A1}, —S(O)(═NR^{m})R^{5A1}, —S(O)(═NR^{m})N(R^{5A1})₂, —P(O)(R^{5A1})₂, methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A} is independently optionally substituted with 1, 2, 3, 4, 5, or 6 R^{5AG} substituents; or the methyl of R^{5A} is substituted with 1, 2, or 3 R^{5AG} substituents.

In some embodiments, R^{5A} is —NHR^{5A12} or —OR^{5A11}.

In some embodiments, R^{5A} is —OR^{5A11}.

In some embodiments, R^{5A} is —NHR^{5A12}.

In some embodiments of any of Formulae I, II, III, IV, V, Va, VI, VIa, VII, and VIIa each R^{5A12} is independently C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A12} is independently optionally substituted with 1, 2, 3, 4, 5, or 6 R^{G1} substituents; or two R^{5A12} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

In some embodiments of any of Formulae I, II, III, IV, V, Va, VI, and VIa, each R^{5A12} is independently C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A12} is independently optionally substituted with 1, 2, 3, 4, 5, or 6 R^{G1} substituents; or two R^{5A12} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

In some embodiments of any of Formulae I, II, and III, each R^{5A12} is independently C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A12} is independently optionally substituted with 1, 2, 3, 4, 5, or 6 R^{G1} substituents; or two R^{5A12} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

In some embodiments, each R^{5A12} is independently 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A12} is independently optionally substituted with 1, 2, 3, 4, 5, or 6 R^{G1} substituents.

In some embodiments, each R^{5A12} is independently phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the phenyl, 8- to 10-membered bicyclic aryl, 5- to 6-membered monocyclic heteroaryl, and 8- to 10-membered bicyclic heteroaryl of R^{5A12} is independently optionally substituted with 1, 2, 3, 4, 5, or 6 R^{G1} substituents.

In some embodiments, each R^{5A12} is independently phenyl or 5- to 6-membered monocyclic heteroaryl having 1-4 heteroatoms independently selected from nitrogen, oxygen, and sulfur, wherein each of the phenyl and 5- to 6-membered monocyclic heteroaryl of R^{5A12} is independently optionally substituted with 1, 2, 3, 4, 5, or 6 R^{G1} substituents.

In some embodiments, each R^{5A12} is independently phenyl or pyridinyl, each of which is optionally substituted with 1, 2, 3, 4, 5, or 6 independently selected R^{G1} substituents. In some embodiments, each R^{5A12} is independently phenyl optionally substituted with 1, 2, 3, 4, or 5 independently selected R^{G1} substituents.

In some embodiments, a single instance of R^{5A12} is phenyl or pyridinyl, each of which is optionally substituted with 1, 2, or 3 independently selected R^{G1} substituents. In some embodiments, a single instance of R^{5A12} is phenyl or pyridinyl, each of which is optionally substituted with 1 or 2 independently selected R^{G1} substituents. In some embodiments, a single instance of R^{5A12} is phenyl or pyridinyl, each of which is optionally substituted with 1 R^{G1} substituent.

In some embodiments, each R^{5A12} is independently phenyl optionally substituted with 1, 2, 3, 4, 5, or 6 independently selected R^{G1} substituents. In some embodiments, a single instance of R^{5A12} is phenyl substituted with 1, 2, or 3 independently selected R^{G1} substituents. In some embodiments, a single instance of R^{5A12} is phenyl substituted with 1 or 2 independently selected R^{G1} substituents. In some embodiments, a single instance of R^{5A12} is phenyl substituted with 1 R^{G1} substituent.

In some embodiments, each R^{5A12} is independently pyridinyl optionally substituted with 1, 2, 3, 4, 5, or 6 independently selected R^{G1} substituents. In some embodiments, a single instance of R^{5A12} is pyridinyl substituted with 1, 2, or 3 independently selected R^{G1} substituents. In some embodiments, a single instance of R^{5A12} is pyridinyl, which is optionally substituted with 1 or 2 independently selected R^{G1} substituents. In some embodiments, a single instance of R^{5A12} is pyridinyl substituted with 1 or 2 independently selected R^{G1} substituents. In some embodiments, a single instance of R^{5A12} is pyridinyl substituted with 1 R^{G1} substituent.

In some embodiments of Formula I, each R^{5A1} is independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A1} is independently optionally substituted with 1, 2, 3, 4, 5, or 6 R^{G1} substituents; or two R^{5A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

As described above, in some embodiments of Formula I, R^{5A11} is methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A11} is independently optionally substituted with 1, 2, 3, 4, 5, or 6 R^{G1} substituents; or the methyl of R^{5A11} is substituted with 1, 2, or 3 R^{G1} substituents.

In some embodiments, R^{5A11} is 5- to 6-membered monocyclic heteroaryl having 1-4 heteroatoms independently selected from nitrogen, oxygen, and sulfur, which is independently optionally substituted with 1, 2, 3, 4, 5, or 6 R^{G1} substituents.

In some embodiments, R^{5A11} is 5- to 6-membered monocyclic heteroaryl having 1-4 heteroatoms independently selected from nitrogen, oxygen, and sulfur, which is independently optionally substituted with 1 or 2 R^{G1} substituents.

In some embodiments, R^{5A11} is pyridyl, which is optionally substituted with 1 or 2 independently selected R^{G1} substituents.

In some embodiments of Formula I, each R^{5AG} is independently halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments of any of Formulae I, II, III, IV, V, Va, VI, VIa, VII, VIIa, and VIIb, each R^{G1} is independently halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR¹¹)N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments of any of Formulae I, II, III, IV, V, Va, VI, and VIa, each R^{G1} is independently halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments of any of Formulae I, II, and III, each R^{G1} is independently halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments, each R^{G1} is independently selected from halogen, C₁₋₆ alkyl, phenyl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl, having 1-4 heteroatoms independently selected from nitrogen, oxygen, and sulfur, 8- to 10-membered bicyclic heteroaryl having 1-4 heteroatoms independently selected from nitrogen, oxygen, and sulfur, —C(O)OR, —C(O)N(R)₂, and —C(O)NR(OR), wherein each C₁₋₆ alkyl, phenyl, 3- to 7-membered saturated or partially unsaturated monocyclic heterocyclyl, 5- to 10-membered saturated or partially unsaturated bicyclic heterocyclyl, 5- to 6-membered monocyclic heteroaryl, and 8- to 10-membered bicyclic heteroaryl is optionally substituted by 1, 2, 3, or 4 independently selected R^{AG1} substituents.

In some embodiments, each R^{G1} is independently selected from halogen, C₁₋₆ alkyl, phenyl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl, having 1-4 heteroatoms independently selected from nitrogen, oxygen, and sulfur, 8- to 10-membered bicyclic heteroaryl having 1-4 heteroatoms independently selected from nitrogen, oxygen, and sulfur, —C(O)OR, —C(O)N(R)₂, and —C(O)NR(OR), wherein each C₁₋₆ alkyl, phenyl, 3- to 7-membered saturated or partially unsaturated monocyclic heterocyclyl, 5- to 10-membered saturated or partially unsaturated bicyclic heterocyclyl, 5- to 6-membered monocyclic heteroaryl, and 8- to 10-membered bicyclic heteroaryl is optionally substituted by 1 or 2 independently selected R^{AG1} substituents.

In some embodiments, each R^{G1} is independently selected from halogen, C₁₋₆ alkyl, phenyl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl, having 1-4 heteroatoms independently selected from nitrogen, oxygen, and sulfur, —C(O)OR, —C(O)N(R)₂, and —C(O)NR(OR), wherein each C₁₋₆ alkyl, phenyl, 3- to 7-membered saturated or partially unsaturated monocyclic heterocyclyl, 5- to 10-membered saturated or partially unsaturated bicyclic heterocyclyl, 5- to 6-membered monocyclic heteroaryl, is optionally substituted by 1, 2, 3, or 4 independently selected R^{AG1} substituents.

In some embodiments, each R^{AG1} is independently selected from halogen, —(CH₂)₀₋₄R^{o}, —(CH₂)₀₋₄OR^{o}, —(CH₂)₀₋₄C(O)NR^{o}₂, —(CH₂)₀₋₄C(O)OR^{o}, —(CH₂)₀₋₄C(O)R^{o}, —O(CH₂)₀₋₄R^{o}, CN, —(CH₂)₀₋₄C(O)NR^{o}₂, and —C(O)N(OR^{o})R^{o}, wherein each R^{o} is independently hydrogen, C₁₋₆ alkyl, or 3- to 6-membered saturated ring having 0-4 heteroatoms independently selected from nitrogen, oxygen, or sulfur.

In some embodiments, each R^{AG1} is independently selected from halogen, —(CH₂)₀₋₄R^{o}, —(CH₂)₀₋₄OR^{o}, —(CH₂)₀₋₄C(O)NR^{o}₂, —(CH₂)₀₋₄C(O)OR^{o}, —(CH₂)₀₋₄C(O)R^{o}, and —C(O)N(OR^{o})R^{o}.

In some embodiments, each R^{o} is independently hydrogen or C₁₋₆ alkyl. In some embodiments, each R^{o} is independently hydrogen or C₁₋₃ alkyl. In some embodiments, each R^{o} is independently hydrogen or methyl.

In some embodiments, each R^{AG1} is independently selected from halogen, —(CH₂)₀₋₄R^{o}, —(CH₂)₀₋₄OR^{o}, —(CH₂)₀₋₄C(O)NR^{o}₂, —(CH₂)₀₋₄C(O)OR^{o}, —(CH₂)₀₋₄C(O)R^{o}, and —C(O)N(OR^{o})R^{o}, wherein each R^{o} is independently hydrogen or C₁₋₆ alkyl.

In some embodiments, each R^{AG1} is independently selected from fluoro, methyl, ethyl, hydroxyethyl, —COOH, methylaminocarbonyl, dimethylaminocarbonyl, methylcarbonyl, methoxyaminocarbonyl, cyano, cyclopropyl, hydroxy, methoxy, dimethylamino, dimethylaminomethyl, and tetrahydropyranyl.

In some embodiments, each R^{AG1} is independently selected from fluoro, methyl, hydroxyethyl, —COOH, methylaminocarbonyl, dimethylaminocarbonyl, methylcarbonyl, and methoxyaminocarbonyl.

In some embodiments, each R^{G1} is independently selected from halogen, C₁₋₆ aliphatic, and —C(O)OR. In some embodiments, each R^{G1} is independently selected from halogen, C₁₋₆ alkyl, and —C(O)OR. In some embodiments, each R^{G1} is independently selected from halogen, C₁₋₃ alkyl, and —C(O)OR. In some embodiments, each R^{G1} is independently selected from halogen, C₁₋₆ alkyl, and —C(O)OH. In some embodiments, each R^{G1} is independently selected from halogen, C₁₋₃ alkyl, and —C(O)OH. In some embodiments, each R^{G1} is independently selected from fluoro, chloro, methyl, and —C(O)OH.

In some embodiments, each R^{G1} is independently selected from methyl, chloro, pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, tetrazolyl, triazolyl, oxadiazolyl, indazolyl, quinolinyl, piperidinyl, piperazinyl, morpholinyl, isoindolinonyl, hexahydro-3H-oxazolo[3,4-a]pyrazin-3-onyl, 4,5,6,7-tetrahydropyrazolo[1,5-a]pyrazinyl, —C(O)OR, —C(O)N(R)₂, and —C(O)NR(OR), wherein the pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, tetrazolyl, triazolyl, oxadiazolyl, indazolyl, quinolinyl, piperidinyl, piperazinyl, morpholinyl, isoindolinonyl, hexahydro-3H-oxazolo[3,4-a]pyrazin-3-onyl, 4,5,6,7-tetrahydropyrazolo[1,5-a]pyrazinyl, are each optionally substituted by 1 or 2 R^{AG1} substituents.

In some embodiments, each R^{G1} is independently selected from methyl, chloro, pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, tetrazolyl, triazolyl, oxadiazolyl, indazolyl, quinolinyl, piperidinyl, piperazinyl, morpholinyl, isoindolinonyl, hexahydro-3H-oxazolo [3,4-a]pyrazin-3-onyl, 4,5,6,7-tetrahydropyrazolo[1,5-a]pyrazinyl, —C(O)OR, —C(O)N(R)₂, and —C(O)NR(OR), wherein the pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, tetrazolyl, triazolyl, oxadiazolyl, indazolyl, quinolinyl, piperidinyl, piperazinyl, morpholinyl, isoindolinonyl, hexahydro-3H-oxazolo[3,4-a]pyrazin-3-onyl, 4,5,6,7-tetrahydropyrazolo[1,5-a]pyrazinyl, are each optionally substituted by 1 or 2 R^{AG1} substituents independently selected from fluoro, methyl, ethyl, hydroxyethyl, —COOH, methylaminocarbonyl, dimethylaminocarbonyl, methylcarbonyl, methoxyaminocarbonyl, cyano, cyclopropyl, hydroxy, methoxy, dimethylamino, dimethylaminomethyl, and tetrahydropyranyl.

In some embodiments, each R^{G1} is independently selected from methyl, chloro, pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, tetrazolyl, —C(O)OR, —C(O)N(R)₂, and —C(O)NR(OR), wherein the pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, and tetrazolyl, are each optionally substituted by 1 or 2 R^{AG1} substituents independently selected from fluoro, methyl, hydroxyethyl, —COOH, methylaminocarbonyl, dimethylaminocarbonyl, methylcarbonyl, and methoxyaminocarbonyl.

In some embodiments, R^{G1} is —C(O)OR. In some embodiments, R^{G1} is —C(O)OH. In some embodiments, one R^{G1} is —C(O)OR and a second R^{G1} is selected from halogen and C₁₋₆ aliphatic. In some embodiments, one R^{G1} is —C(O)OR and a second R^{G1} is selected from halogen and C₁₋₆ alkyl. In some embodiments, one R^{G1} is —C(O)OR and a second R^{G1} is selected from halogen and C₁₋₃ alkyl. In some embodiments, one R^{G1} is —C(O)OH and a second R^{G1} is selected from halogen and C₁₋₆ alkyl. In some embodiments, one R^{G1} is —C(O)OH and a second R^{G1} is selected from halogen and C₁₋₃ alkyl. In some embodiments, one R^{G1} is —C(O)OH and a second R^{G1} is selected from fluoro, chloro, and methyl.

In some embodiments of any of Formulae I, II, III, IV, V, Va, VI, VIa, VII, VIIa, and VIIb each R is independently hydrogen or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or two R when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

In some embodiments of any of Formulae I, II, III, IV, V, Va, VI, and VIa each R is independently hydrogen or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or two R when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

As described above, in some embodiments of any of Formulae I, II, and III, each R is independently hydrogen or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or two R when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

In some embodiments, each R is independently selected from hydrogen, C₁₋₆ alkyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein each 3- to 7-membered saturated or partially unsaturated monocyclic heterocyclyl and 5- to 6-membered monocyclic heteroaryl is optionally substituted by C₁₋₆ alkyl.

In some embodiments, each R is independently selected from hydrogen, methyl, methylpiperidinyl, and pyridinyl.

In some embodiments, each R is independently selected from hydrogen, methyl, methylpiperidinyl, and pyridinyl. In some embodiments, R is hydrogen.

In some embodiments:
each R^{G1} is independently selected from methyl, chloro, pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, tetrazolyl, triazolyl, oxadiazolyl, indazolyl, quinolinyl, piperidinyl, piperazinyl, morpholinyl, isoindolinonyl, hexahydro-3H-oxazolo[3,4-a]pyrazin-3-onyl, 4,5,6,7-tetrahydropyrazolo[1,5-a]pyrazinyl, —C(O)OR, —C(O)N(R)₂, and —C(O)NR(OR), wherein the pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, tetrazolyl, triazolyl, oxadiazolyl, indazolyl, quinolinyl, piperidinyl, piperazinyl, morpholinyl, isoindolinonyl, hexahydro-3H-oxazolo[3,4-a]pyrazin-3-onyl, 4,5,6,7-tetrahydropyrazolo[1,5-a]pyrazinyl, are each optionally substituted by 1 or 2 R^{AG1} substituents independently selected from fluoro, methyl, ethyl, hydroxyethyl, —COOH, methylaminocarbonyl, dimethylaminocarbonyl, methylcarbonyl, methoxyaminocarbonyl, cyano, cyclopropyl, hydroxy, methoxy, dimethylamino, dimethylaminomethyl, and tetrahydropyranyl; and
each R is independently selected from hydrogen, methyl, methylpiperidinyl, and pyridinyl

In some embodiments:
each R^{G1} is independently selected from methyl, chloro, pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, tetrazolyl, —C(O)OR, —C(O)N(R)₂, and —C(O)NR(OR), wherein the pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, and tetrazolyl, are each optionally substituted by 1 or 2 R^{AG1} substituents independently selected from fluoro, methyl, hydroxyethyl, —COOH, methylaminocarbonyl, dimethylaminocarbonyl, methylcarbonyl, and methoxyaminocarbonyl; and
each R is independently selected from hydrogen, C₁₋₆ alkyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein each 3- to 7-membered saturated or partially unsaturated monocyclic heterocyclyl and 5- to 6-membered monocyclic heteroaryl is optionally substituted by C₁₋₆ alkyl.

In some embodiments:
each R^{G1} is independently selected from methyl, chloro, pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, tetrazolyl, —C(O)OR, —C(O)N(R)₂, and —C(O)NR(OR), wherein the pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, and tetrazolyl, are each optionally substituted by 1 or 2 R^{AG1} substituents independently selected from fluoro, methyl, hydroxyethyl, —COOH, methylaminocarbonyl, dimethylaminocarbonyl, methylcarbonyl, and methoxyaminocarbonyl; and
each R is independently selected from hydrogen, methyl, methylpiperidinyl, and pyridinyl.

In some embodiments of any of Formulae I, II, III, IV, V, Va, VI, VIa, VII, VIIa, and VIIb, each R^{m} is independently —OH, —CN, or R. In some embodiments of any of Formulae I, II, III, IV, V, Va, VI, and VIa each R^{m} is independently —OH, —CN, or R. As described above, in some embodiments of any of Formulae I, II, and III, each R^{m} is independently —OH, —CN, or R.

In some embodiments of any of Formulae I, II, III, IV, V, Va, VI, VIa, VII, VIIa, and VIIb, each n is 0, 1, 2, 3, or 4. In some embodiments of any of Formulae I, II, III, IV, V, Va, VI, and VIa, each n is 0, 1, 2, 3, or 4. As described above, in some embodiments of any of Formulae I, II, and III, each n is 0, 1, 2, 3, or 4. In some embodiments, n is 0. In some embodiments, n is 1, 2, 3, or 4. In some embodiments, n is 0 or 1. In some embodiments, n is 1.

In some embodiments, the compound of Formula I is a compound of Formula V:

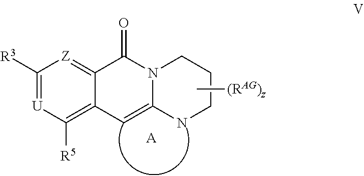

or a pharmaceutically acceptable salt thereof, wherein z is 0, 1, 2, or 3, and each of U, Z, Ring A, R³, R⁵, and R^{AG} are as defined above for Formula I and described in classes and subclasses herein, both singly and in combination.

In some embodiments, the compound of Formula I is a compound of Formula Va:

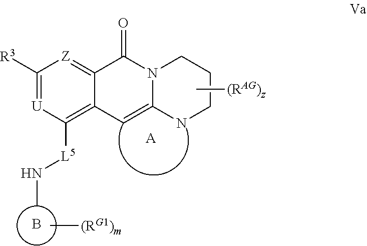

or a pharmaceutically acceptable salt thereof, wherein z is 0, 1, 2, or 3, and each of U, Z, Ring A, Ring B, R³, L⁵, R^{G1}, R^{AG}, and m are as defined above for Formula I and described in classes and subclasses herein, both singly and in combination.

In some embodiments, the compound of Formula I is a compound of Formula VI:

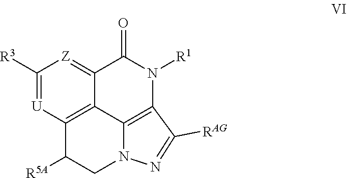

or a pharmaceutically acceptable salt thereof, wherein each of U, Z, R¹, R³, R^{5A}, and R^{AG} are as defined above for Formula I and described in classes and subclasses herein, both singly and in combination.

In some embodiments, the compound of Formula I is a compound of Formula VIa:

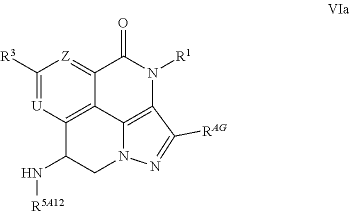

or a pharmaceutically acceptable salt thereof, wherein each of U, Z, R¹, R³, R^{5A12}, and R^{AG} are as defined above for Formula I and described in classes and subclasses herein, both singly and in combination.

In some embodiments, the compound is of Formula VII:

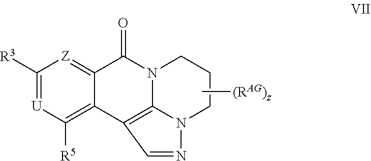

or a pharmaceutically acceptable salt thereof, wherein z is 0, 1, 2, or 3, and each of U, Z, R³, R⁵, and R^{AG} are as defined above for Formula I and described in classes and subclasses herein, both singly and in combination.

In some embodiments, the compound is of Formula VIIa:

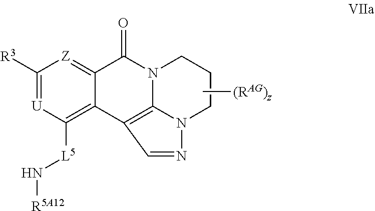

or a pharmaceutically acceptable salt thereof, wherein z is 0, 1, 2, or 3, and each of U, Z, R³, L⁵, R^{5A12}, and R^{AG} are as defined above for Formula I and described in classes and subclasses herein, both singly and in combination.

In some embodiments, the compound is of Formula VIIb:

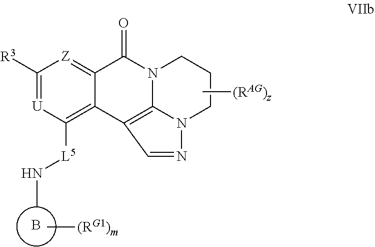

or a pharmaceutically acceptable salt thereof, wherein z is 0, 1, 2, or 3, and each of U, Z, Ring B, R³, L⁵, R^{G1}, R^{AG}, and m are as defined above for Formula I and described in classes and subclasses herein, both singly and in combination.

In some embodiments, the present disclosure provides compounds selected from Table 1, or a pharmaceutically acceptable salt thereof.

TABLE 1

| | |
|---|---|
| 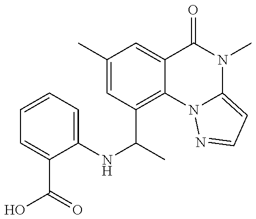 [SMILES: Cc1cc(C(C)Nc2ccccc2C(=O)O)c2c(c1)c(=O)n(C)c1ccnn21] | I-1 |
| 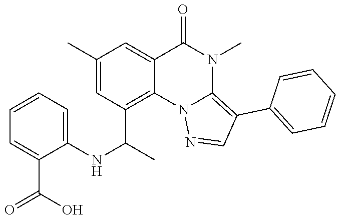 [SMILES: Cc1cc(C(C)Nc2ccccc2C(=O)O)c2c(c1)c(=O)n(C)c1c(-c3ccccc3)cnn21] | I-2 |
| 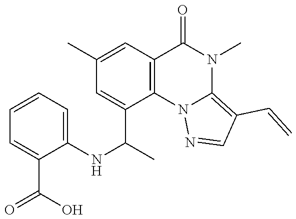 [SMILES: C=Cc1cnn2c3c(C(C)Nc4ccccc4C(=O)O)cc(C)cc3c(=O)n(C)c12] | I-3 |

In some embodiments, the compound provided herein is selected from:
2-((1-(4,7-dimethyl-5-oxo-4,5-dihydropyrazolo[1,5-a]quinazolin-9-yl)ethyl)amino)benzoic acid;
2-((1-(4,7-dimethyl-5-oxo-3-phenyl-4,5-dihydropyrazolo[1,5-a]quinazolin-9-yl)ethyl)amino)benzoic acid;
2-((1-(4,7-dimethyl-5-oxo-3-vinyl-4,5-dihydropyrazolo[1,5-a]quinazolin-9-yl)ethyl)amino)benzoic acid;
2-((1-(4,7-dimethyl-5-oxo-4,5-dihydroimidazo[1,5-a]quinazolin-9-yl)ethyl)amino)benzoic acid;
2-((1-(3-ethyl-4,7-dimethyl-5-oxo-4,5-dihydroimidazo[1,5-a]quinazolin-9-yl)ethyl)amino)benzoic acid;
2-((1-(4,7-dimethyl-5-oxo-3-(pyridin-2-yl)-4,5-dihydroimidazo[1,5-a]quinazolin-9-yl)ethyl)amino)benzoic acid;
2-((1-(3,4,7-trimethyl-5-oxo-4,5-dihydroimidazo[1,5-a]quinazolin-9-yl)ethyl)amino)benzoic acid;
6-methyl-3-((1-(3,4,7-trimethyl-5-oxo-4,5-dihydroimidazo[1,5-a]quinazolin-9-yl)ethyl)amino)picolinic acid;
2-((1-(7-chloro-3-ethyl-4-methyl-5-oxo-4,5-dihydroimidazo[1,5-a]quinazolin-9-yl)ethyl)amino)benzoic acid;
2-((1-(7-chloro-4-methyl-5-oxo-4,5-dihydroimidazo[1,5-a]quinazolin-9-yl)ethyl)amino)benzoic acid;
2-((1-(7-chloro-3,4-dimethyl-5-oxo-4,5-dihydroimidazo[1,5-a]quinazolin-9-yl)ethyl)amino)benzoic acid;
2-((1-(7-chloro-4-ethyl-5-oxo-4,5-dihydroimidazo[1,5-a]quinazolin-9-yl)ethyl)amino)benzoic acid;
2-((1-(7-chloro-3,4-diethyl-5-oxo-4,5-dihydroimidazo[1,5-a]quinazolin-9-yl)ethyl)amino)benzoic acid;
2-((1-(4,7-dimethyl-5-oxo-4,5-dihydroimidazo[1,2-a]quinazolin-9-yl)ethyl)amino)benzoic acid;
2-((1-(4,7-dimethyl-5-oxo-4,5-dihydro-[1,2,4]triazolo[1,5-a]quinazolin-9-yl)ethyl)amino)benzoic acid;
2-((1-(3,4,7-trimethyl-5-oxo-4,5-dihydro-3H-pyrazolo[3,4-c]isoquinolin-9-yl)ethyl)amino)benzoic acid;
6-chloro-3-((1-(3,4,7-trimethyl-5-oxo-4,5-dihydro-3H-pyrazolo[3,4-c]isoquinolin-9-yl)ethyl)amino)picolinic acid;
2-((1-(3-ethyl-4,7-dimethyl-5-oxo-4,5-dihydro-3H-pyrazolo[3,4-c]isoquinolin-9-yl)ethyl)amino)benzoic acid;
6-chloro-3-((1-(3-ethyl-4,7-dimethyl-5-oxo-4,5-dihydro-3H-pyrazolo[3,4-c]isoquinolin-9-yl)ethyl)amino)picolinic acid;
3-((1-(3-ethyl-4,7-dimethyl-5-oxo-4,5-dihydro-3H-pyrazolo[3,4-c]isoquinolin-9-yl)ethyl)amino)-6-methylpicolinic acid;
2-((1-(4-ethyl-3,7-dimethyl-5-oxo-4,5-dihydro-3H-pyrazolo[3,4-c]isoquinolin-9-yl)ethyl)amino)benzoic acid;
6-chloro-3-((1-(4-ethyl-3,7-dimethyl-5-oxo-4,5-dihydro-3H-pyrazolo[3,4-c]isoquinolin-9-yl)ethyl)amino)picolinic acid;
2-((1-(3,4-diethyl-7-methyl-5-oxo-4,5-dihydro-3H-pyrazolo[3,4-c]isoquinolin-9-yl)ethyl)amino)benzoic acid;
2-((1-(3,4-diethyl-7-methyl-5-oxo-4,5-dihydro-3H-pyrazolo[3,4-c]isoquinolin-9-yl)ethyl)amino)-5-fluorobenzoic acid;
3-((1-(3,4-diethyl-7-methyl-5-oxo-4,5-dihydro-3H-pyrazolo[3,4-c]isoquinolin-9-yl)ethyl)amino)-6-methylpicolinic acid;
2-((1-(4,7-dimethyl-5-oxo-2-propyl-4,5-dihydro-2H-pyrazolo[3,4-c]isoquinolin-9-yl)ethyl)amino)benzoic acid;
2-((1-(4,7-dimethyl-5-oxo-2-(tetrahydro-2H-pyran-4-yl)-4,5-dihydro-2H-pyrazolo[3,4-c]isoquinolin-9-yl)ethyl)amino)benzoic acid;
2-((1-(4,7-dimethyl-5-oxo-2-phenethyl-4,5-dihydro-2H-pyrazolo[3,4-c]isoquinolin-9-yl)ethyl)amino)benzoic acid;
2-((1-(2-(2-fluoropyridin-4-yl)-4,7-dimethyl-5-oxo-4,5-dihydro-2H-pyrazolo[3,4-c]isoquinolin-9-yl)ethyl)amino)benzoic acid;
2-((1-(4,7-dimethyl-5-oxo-2-(pyrimidin-5-yl)-4,5-dihydro-2H-pyrazolo[3,4-c]isoquinolin-9-yl)ethyl)amino)benzoic acid;
6-chloro-3-((1-(4,7-dimethyl-5-oxo-2-(o-tolyl)-4,5-dihydro-2H-pyrazolo[3,4-c]isoquinolin-9-yl)ethyl)amino)picolinic acid;
2-[1-(3-methoxy-5,8-dimethyl-6-oxo-benzo[c][1,8]naphthyridin-10-yl)ethylamino]benzoic acid;
2-[1-[3-(dimethylamino)-5,8-dimethyl-6-oxo-benzo[c][1,8]naphthyridin-10-yl]ethylamino]benzoic acid;
2-[1-[3-(2-methoxyethoxy)-5,8-dimethyl-6-oxo-benzo[c][1,8]naphthyridin-10-yl]ethylamino]benzoic acid;
2-((1-(7-chloro-3,4-dimethyl-5-oxo-4,5-dihydro-3H-pyrazolo[3,4-c]isoquinolin-9-yl)ethyl)amino)benzoic acid;
2-((1-(8-methyl-6-oxo-4,5-dihydro-3H,6H-2,2a,5a-triazaaceanthrylen-10-yl)ethyl)amino)benzoic acid;
6-methyl-3-((1-(8-methyl-6-oxo-4,5-dihydro-3H,6H-2,2a,5a-triazaaceanthrylen-10-yl)ethyl)amino)picolinic acid;
2-((1-(3,8-dimethyl-6-oxo-4,5-dihydro-3H,6H-2,2a,5a-triazaaceanthrylen-10-yl)ethyl)amino)benzoic acid;
6-chloro-3-((1-(3,8-dimethyl-6-oxo-4,5-dihydro-3H,6H-2,2a,5a-triazaaceanthrylen-10-yl)ethyl)amino)picolinic acid;
2-ethyl-3,6-dimethyl-8-((6-methyl-[2,4′-bipyridin]-3-yl)amino)-8,9-dihydrobenzo[de]pyrazolo[4,5,1-ij][1,7]naphthyridin-4(3H)-one;
9-(1-((6-chloro-1′-methyl-6′-oxo-1′,6′-dihydro-[2,3′-bipyridin]-3-yl)amino)ethyl)-3-ethyl-4,7-dimethyl-3,4-dihydro-5H-pyrazolo[3,4-c]isoquinolin-5-one;
4-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)-2-fluoro-N-methylbenzamide;
9-(1-((6-chloro-2-(pyrimidin-4-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(pyrimidin-5-yl)pyridin-3-yl)amino)ethyl)-3-ethyl-4,7-dimethyl-3,4-dihydro-5H-pyrazolo[3,4-c]isoquinolin-5-one;
9-(1-((6-chloro-3′-fluoro-[2,4′-bipyridin]-3-yl)amino)ethyl)-3-ethyl-4,7-dimethyl-3,4-dihydro-5H-pyrazolo[3,4-c]isoquinolin-5-one;
4-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)-N,N-dimethylbenzamide;
4-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)benzoic acid;
9-(1-((6-chloro-2-(1-methyl-1H-pyrazol-5-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(1,5-dimethyl-1H-pyrazol-4-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(1H-pyrazol-4-yl)pyridin-3-yl)amino)ethyl)-3-ethyl-4,7-dimethyl-3,4-dihydro-5H-pyrazolo[3,4-c]isoquinolin-5-one;
3-ethyl-4,7-dimethyl-9-(1-((6-methyl-2-(1-methyl-1H-pyrazol-4-yl)pyridin-3-yl)amino)ethyl)-3,4-dihydro-5H-pyrazolo[3,4-c]isoquinolin-5-one;
3-ethyl-4,7-dimethyl-9-(1-((6-methyl-2-(1H-pyrazol-4-yl)pyridin-3-yl)amino)ethyl)-3,4-dihydro-5H-pyrazolo[3,4-c]isoquinolin-5-one;
3-ethyl-4,7-dimethyl-9-(1-((6-methyl-2-(1-methyl-1H-pyrazol-5-yl)pyridin-3-yl)amino)ethyl)-3,4-dihydro-5H-pyrazolo[3,4-c]isoquinolin-5-one;
4-(3-((1-(3-ethyl-4,7-dimethyl-5-oxo-4,5-dihydro-3H-pyrazolo[3,4-c]isoquinolin-9-yl)ethyl)amino)-6-methylpyridin-2-yl)-2-fluoro-N-methylbenzamide;
3-ethyl-9-(1-((2-(1-(2-hydroxyethyl)-1H-pyrazol-4-yl)phenyl)amino)ethyl)-4,7-dimethyl-3,4-dihydro-5H-pyrazolo[3,4-c]isoquinolin-5-one;
9-(1-((2-(4-acetylpiperazin-1-yl)phenyl)amino)ethyl)-3-ethyl-4,7-dimethyl-3,4-dihydro-5H-pyrazolo[3,4-c]isoquinolin-5-one;
9-(1-((2-(5,6-dihydro-[1,2,4]triazolo[4,3-a]pyrazin-7(8H)-yl)phenyl)amino)ethyl)-3-ethyl-4,7-dimethyl-3,4-dihydro-5H-pyrazolo[3,4-c]isoquinolin-5-one;
3-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(4-(2,2-difluoroethyl)-3-ethyl-7-methyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3-cyclopentyl-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3-isopropyl-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(7-chloro-3-ethyl-4-methyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(7-chloro-3-(cyclopropylmethyl)-4-methyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(4,7-dimethyl-5-oxo-3-propyl-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(4,7-dimethyl-5-oxo-3-(tetrahydrofuran-3-yl)-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3-(2-methoxyethyl)-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3-(cyclopropylmethyl)-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3-ethyl-4-methyl-5-oxo-7-(trifluoromethyl)-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3,8-dimethyl-6-oxo-4,5-dihydro-3H,6H-2,2a,5a-triazaaceanthrylen-10-yl)ethyl)amino)pyridin-2-yl)-1,2,4-oxadiazol-5(4H)-one;
3-(6-chloro-3-((1-(3-ethyl-4,7-dimethyl-5-oxo-4,5-dihydroimidazo[1,5-a]quinazolin-9-yl)ethyl)amino)pyridin-2-yl)-1,2,4-oxadiazol-5(4H)-one;
9-(1-((2-(1H-tetrazol-5-yl)pyridin-3-yl)amino)ethyl)-3-ethyl-4,7-dimethyl-3,4-dihydro-5H-pyrazolo[3,4-c]isoquinolin-5-one;
6-chloro-3-((1-(3-ethyl-4,7-dimethyl-5-oxo-4,5-dihydro-3H-pyrazolo[3,4-c]isoquinolin-9-yl)ethyl)amino)-N-(1-methylpiperidin-4-yl)picolinamide;
6-chloro-3-((1-(3-ethyl-4,7-dimethyl-5-oxo-4,5-dihydro-3H-pyrazolo[3,4-c]isoquinolin-9-yl)ethyl)amino)-N-(pyridin-4-yl)picolinamide;
6-chloro-3-((1-(3-ethyl-4,7-dimethyl-5-oxo-4,5-dihydro-3H-pyrazolo[3,4-c]isoquinolin-9-yl)ethyl)amino)-N-methoxypicolinamide;
9-(1-((6-chloro-3′-fluoro-[2,4′-bipyridin]-3-yl)amino)-2-hydroxyethyl)-3-ethyl-4,7-dimethyl-3,4-dihydro-5H-pyrazolo[3,4-c]isoquinolin-5-one;
9-(1-((6-chloro-2-(1-methyl-1H-pyrazol-4-yl)pyridin-3-yl)amino)-2-hydroxyethyl)-3-ethyl-4,7-dimethyl-3,4-dihydro-5H-pyrazolo[3,4-c]isoquinolin-5-one;
9-(1-((6-chloro-2-(1,5-dimethyl-1H-pyrazol-4-yl)pyridin-3-yl)amino)-2-hydroxyethyl)-3-ethyl-4,7-dimethyl-3,4-dihydro-5H-pyrazolo[3,4-c]isoquinolin-5-one;
9-(1-((6-chloro-2-(1-(1-methylpiperidin-4-yl)-1H-pyrazol-4-yl)pyridin-3-yl)amino)ethyl)-3-ethyl-4,7-dimethyl-3,4-dihydro-5H-pyrazolo[3,4-c]isoquinolin-5-one;
9-(1-((2-(4-acetylpiperazin-1-yl)-6-chloropyridin-3-yl)amino)ethyl)-3-ethyl-4,7-dimethyl-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-2-(1-(2-hydroxyethyl)-1H-1,2,4-triazol-3-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-2-(1H-pyrazol-4-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-1′-methyl-6′-oxo-1′,6′-dihydro-[2,3′-bipyridin]-3-yl)amino)ethyl)-4,7-dimethyl-3-(1-methylazetidin-3-yl)-3,4-dihydro-5H-pyrazolo[3,4-c]isoquinolin-5-one;
5-(5-chloro-2-((1-(4,7-dimethyl-3-(1-methylazetidin-3-yl)-5-oxo-4,5-dihydro-3H-pyrazolo[3,4-c]isoquinolin-9-yl)ethyl)amino)phenyl)-N-methylpicolinamide;
9-(1-((6-chloro-2-(2-methyl-1-oxoisoindolin-5-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-2-(1-methyl-1H-indazol-5-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2-hydroxyethyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-cyclopropylazetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-((S)-2-hydroxypropyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-((R)-2-hydroxypropyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2-hydroxy-2-methylpropyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2,2-difluoroethyl) azetidin-3-yl)-4,7-dimethyl-3,4-dihydro-5H-pyrazolo[3,4-c]isoquinolin-5-one;
2-(3-(9-(1-((6-chloro-2-(1-methyl-1H-1,2,4-triazol-3-yl)pyridin-3-yl)amino)ethyl)-4,7-dimethyl-5-oxo-4,5-dihydro-3H-pyrazolo[3,4-c]isoquinolin-3-yl) azetidin-1-yl) acetonitrile;
3-(3-(9-(1-((6-chloro-2-(1-methyl-1H-1,2,4-triazol-3-yl)pyridin-3-yl)amino)ethyl)-4,7-dimethyl-5-oxo-4,5-dihydro-3H-pyrazolo[3,4-c]isoquinolin-3-yl) azetidin-1-yl)propanenitrile;
3-(1-acetylazetidin-3-yl)-9-(1-((6-chloro-2-(1-methyl-1H-1,2,4-triazol-3-yl)pyridin-3-yl)amino)ethyl)-4,7-dimethyl-3,4-dihydro-5H-pyrazolo[3,4-c]isoquinolin-5-one;
methyl 3-(9-(1-((6-chloro-2-(1-methyl-1H-1,2,4-triazol-3-yl)pyridin-3-yl)amino)ethyl)-4,7-dimethyl-5-oxo-4,5-dihydro-3H-pyrazolo[3,4-c]isoquinolin-3-yl) azetidine-1-carboxylate;
9-(1-((6-chloro-2-(1-methyl-1H-1,2,4-triazol-3-yl)pyridin-3-yl)amino)ethyl)-4,7-dimethyl-3-(1-(methylsulfonyl) azetidin-3-yl)-3,4-dihydro-5H-pyrazolo[3,4-c]isoquinolin-5-one;
3-(1-acetylazetidin-3-yl)-9-(1-((6-chloro-1′-methyl-6′-oxo-1′,6′-dihydro-[2,3′-bipyridin]-3-yl)amino)ethyl)-4,7-dimethyl-3,4-dihydro-5H-pyrazolo[3,4-c]isoquinolin-5-one;
3-((1-(3-(1-acetylazetidin-3-yl)-4,7-dimethyl-5-oxo-4,5-dihydro-3H-pyrazolo[3,4-c]isoquinolin-9-yl)ethyl)amino)-6-chloro-N-methyl-[2,3′-bipyridine]-6′-carboxamide;
3-(1-acetylazetidin-3-yl)-9-(1-((6-chloro-2-(1-(2-hydroxyethyl)-1H-1,2,4-triazol-3-yl)pyridin-3-yl)amino)ethyl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2-hydroxyacetyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(dimethylglycyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-7-methyl-4-(methyl-d3)-3-((S)-1-methylpyrrolidin-3-yl)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-((1-(2,2-difluoroethyl)piperidin-4-yl)methyl)-4,7-dimethyl-3,4-dihydro-5H-pyrazolo[3,4-c]isoquinolin-5-one;
3-((1-acetylpiperidin-4-yl)methyl)-9-(1-((6-chloro-2-(1-methyl-1H-pyrazol-4-yl)pyridin-3-yl)amino)ethyl)-4,7-dimethyl-3,4-dihydro-5H-pyrazolo[3,4-c]isoquinolin-5-one;
4-((9-(1-((6-chloro-2-(1-methyl-1H-pyrazol-4-yl)pyridin-3-yl)amino)ethyl)-4,7-dimethyl-5-oxo-4,5-dihydro-3H-pyrazolo[3,4-c]isoquinolin-3-yl)methyl)-N,N-dimethylpiperidine-1-carboxamide;
4-((9-(1-((6-chloro-3′-fluoro-[2,4′-bipyridin]-3-yl)amino)ethyl)-4,7-dimethyl-5-oxo-4,5-dihydro-3H-pyrazolo[3,4-c]isoquinolin-3-yl)methyl)-N-ethylpiperidine-1-carboxamide;
9-(1-((6-chloro-3′-fluoro-[2,4′-bipyridin]-3-yl)amino)ethyl)-3-((1-(2,2-difluoroethyl) pyrrolidin-3-yl)methyl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-hydroxyethyl)-4,7-dimethylimidazo[1,5-a]quinazolin-5(4H)-one;
9-(1-((6-chloro-2-(1-methyl-1H-1,2,4-triazol-3-yl)pyridin-3-yl)amino)ethyl)-4,7-dimethyl-3-(pyridin-3-yl) imidazo[1,5-a]quinazolin-5(4H)-one;
9-(1-((6-chloro-2-(1-methyl-1H-1,2,4-triazol-3-yl)pyridin-3-yl)amino)ethyl)-4,7-dimethyl-3-(2-methylpyridin-3-yl) imidazo[1,5-a]quinazolin-5(4H)-one;
9-(1-((6-chloro-2-(1-methyl-1H-1,2,4-triazol-3-yl)pyridin-3-yl)amino)ethyl)-4,7-dimethyl-3-(pyrimidin-5-yl) imidazo[1,5-a]quinazolin-5(4H)-one;
9-(1-((6-chloro-2-(1-methyl-1H-1,2,4-triazol-3-yl)pyridin-3-yl)amino)ethyl)-4,7-dimethyl-3-(2-methylpyridin-4-yl) imidazo[1,5-a]quinazolin-5(4H)-one;
9-(1-((6-chloro-2-(1-methyl-1H-pyrazol-4-yl)pyridin-3-yl)amino)ethyl)-4,7-dimethyl-3-(2-methylpyridin-3-yl) imidazo[1,5-a]quinazolin-5(4H)-one;
9-(1-((6-chloro-2-(1-methyl-1H-pyrazol-4-yl)pyridin-3-yl)amino)ethyl)-4,7-dimethyl-3-(pyrimidin-5-yl) imidazo[1,5-a]quinazolin-5(4H)-one;
4-(9-(1-((6-chloro-2-(1-methyl-1H-pyrazol-4-yl)pyridin-3-yl)amino)ethyl)-4,7-dimethyl-5-oxo-4,5-dihydroimidazo[1,5-a]quinazolin-3-yl)-N-methylpicolinamide;
9-(1-((6-chloro-2-(1-methyl-1H-1,2,4-triazol-3-yl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-(1-methyl-1H-pyrazol-4-yl)pyridin-3-yl)amino)ethyl)-N,N,4,7-tetramethyl-5-oxo-4,5-dihydroimidazo[1,5-a]quinazoline-3-carboxamide;
3-(azetidine-1-carbonyl)-9-(1-((6-chloro-2-(1-methyl-1H-pyrazol-4-yl)pyridin-3-yl)amino)ethyl)-4,7-dimethylimidazo[1,5-a]quinazolin-5(4H)-one;
9-(1-((6-chloro-2-(1-methyl-1H-pyrazol-4-yl)pyridin-3-yl)amino)ethyl)-3-(3-hydroxyazetidine-1-carbonyl)-4,7-dimethylimidazo[1,5-a]quinazolin-5(4H)-one;
9-(1-((6-chloro-2-(1-methyl-1H-1,2,4-triazol-3-yl)pyridin-3-yl)amino)ethyl)-4-(2,2-difluoroethyl)-N,N,7-trimethyl-5-oxo-4,5-dihydroimidazo[1,5-a]quinazoline-3-carboxamide;
9-(1-((6-chloro-2-(3-methyl-1H-pyrazol-4-yl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-(4-cyano-2-fluorophenyl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-(4-cyanophenyl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-morpholinopyridin-3-yl)amino)ethyl)-N,N,4,7-tetramethyl-5-oxo-4,5-dihydroimidazo[1,5-a]quinazoline-3-carboxamide;
9-(1-((4-fluoro-2-(1-methyl-1H-pyrazol-4-yl)phenyl)amino)ethyl)-N,N,4,7-tetramethyl-5-oxo-4,5-dihydroimidazo[1,5-a]quinazoline-3-carboxamide;
9-(1-((6-chloro-2-(1,5-dimethyl-1H-pyrazol-4-yl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-(1,3-dimethyl-1H-pyrazol-4-yl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-(1-methyl-1H-1,2,4-triazol-3-yl)pyridin-3-yl)amino)ethyl)-4,7-dimethyl-3-(1-methylpiperidin-4-yl) imidazo[1,5-a]quinazolin-5(4H)-one;
3-(1-acetylpiperidin-4-yl)-9-(1-((6-chloro-2-(1-methyl-1H-1,2,4-triazol-3-yl)pyridin-3-yl)amino)ethyl)-4,7-dimethylimidazo[1,5-a]quinazolin-5(4H)-one;
ethyl 4-(9-(1-((6-chloro-2-(1-methyl-1H-1,2,4-triazol-3-yl)pyridin-3-yl)amino)ethyl)-4,7-dimethyl-5-oxo-4,5-dihydroimidazo[1,5-a]quinazolin-3-yl)piperidine-1-carboxylate;
9-(1-((6-chloro-2-(1-methyl-1H-pyrazol-4-yl)pyridin-3-yl)amino)ethyl)-4,7-dimethyl-3-(1-methylpiperidin-4-yl) imidazo[1,5-a]quinazolin-5(4H)-one;
9-(1-((6-chloro-2-(1-methyl-1H-pyrazol-4-yl)pyridin-3-yl)amino)ethyl)-4,7-dimethyl-3-(1-methylpyrrolidin-3-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-methylpyrrolidin-3-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-ethylpyrrolidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2-hydroxyethyl) pyrrolidin-3-yl)-4,7-dimethyl-3,4-dihydro-5H-pyrazolo[3,4-c]isoquinolin-5-one;
9-(1-((6-chloro-2-(2-methyl-2H-tetrazol-5-yl)pyridin-3-yl)amino)ethyl)-7-methyl-4-(methyl-d3)-3-(1-methylpyrrolidin-3-yl)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-(1-(2-hydroxyethyl) pyrrolidin-3-yl)-7-methyl-4-(methyl-d3)-3,4-dihydro-5H-pyrazolo[3,4-c]isoquinolin-5-one;
9-(1-((6-chloro-3′-fluoro-[2,4′-bipyridin]-3-yl)amino)ethyl)-3-(1-isopropylpyrrolidin-3-yl)-4,7-dimethyl-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-(1-(2,2-difluoroethyl) pyrrolidin-3-yl)-4,7-dimethyl-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-(1-(2,2-difluoroethyl) pyrrolidin-3-yl)-7-methyl-4-(methyl-d3)-3,4-dihydro-5H-pyrazolo[3,4-c]isoquinolin-5-one;
2-(3-(9-(1-((6-chloro-2-(1-methyl-1H-pyrazol-4-yl)pyridin-3-yl)amino)ethyl)-7-methyl-4-(methyl-d3)-5-oxo-4,5-dihydro-3H-pyrazolo[3,4-c]isoquinolin-3-yl) pyrrolidin-1-yl) acetonitrile;
9-(1-((6-chloro-2-(1-methyl-1H-1,2,4-triazol-3-yl)pyridin-3-yl)amino)ethyl)-4-ethyl-3-(1-(2-hydroxyethyl) pyrrolidin-3-yl)-7-methyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4-ethyl-3-(1-(2-hydroxy-2-methylpropyl) pyrrolidin-3-yl)-7-methyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4-ethyl-3-(1-isopropylpyrrolidin-3-yl)-7-methyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4-ethyl-7-methyl-3-(1-(methylsulfonyl) pyrrolidin-3-yl)-3,4-dihydro-5H-pyrazolo[3,4-c]isoquinolin-5-one;
3-(1-acetylpyrrolidin-3-yl)-9-(1-((6-chloro-2-(1-methyl-1H-1,2,4-triazol-3-yl)pyridin-3-yl)amino)ethyl)-4-ethyl-7-methyl-3,4-dihydro-5H-pyrazolo[3,4-c]isoquinolin-5-one;
methyl 3-(9-(1-((6-chloro-2-(1-methyl-1H-1,2,4-triazol-3-yl)pyridin-3-yl)amino)ethyl)-4-ethyl-7-methyl-5-oxo-4,5-dihydro-3H-pyrazolo[3,4-c]isoquinolin-3-yl) pyrrolidine-1-carboxylate;
3-(9-(1-((6-chloro-3′-fluoro-[2,4′-bipyridin]-3-yl)amino)ethyl)-4,7-dimethyl-5-oxo-4,5-dihydro-3H-pyrazolo[3,4-c]isoquinolin-3-yl)-N,N-dimethylpyrrolidine-1-carboxamide;
3-(3-(9-(1-((6-chloro-2-(1-methyl-1H-pyrazol-4-yl)pyridin-3-yl)amino)ethyl)-7-methyl-4-(methyl-d3)-5-oxo-4,5-dihydro-3H-pyrazolo[3,4-c]isoquinolin-3-yl) pyrrolidin-1-yl)propanenitrile;
3-(3-(9-(1-((6-chloro-2-(2-methyl-2H-tetrazol-5-yl)pyridin-3-yl)amino)ethyl)-7-methyl-4-(methyl-d3)-5-oxo-4,5-dihydro-3H-pyrazolo[3,4-c]isoquinolin-3-yl) pyrrolidin-1-yl)propanenitrile;
9-(1-((6-chloro-2-(1-methyl-1H-pyrazol-4-yl)pyridin-3-yl)amino)ethyl)-3-(1-(2-hydroxyacetyl) pyrrolidin-3-yl)-7-methyl-4-(methyl-d3)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-(1-(dimethylglycyl) pyrrolidin-3-yl)-7-methyl-4-(methyl-d3)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-7-methyl-4-(methyl-d3)-3-(1-(1-methyl-1H-pyrazole-5-carbonyl) pyrrolidin-3-yl)-3,4-dihydro-5H-pyrazolo[3,4-c]isoquinolin-5-one;
1-(3-(9-(1-((6-chloro-2-(1-methyl-1H-pyrazol-4-yl)pyridin-3-yl)amino)ethyl)-7-methyl-4-(methyl-d3)-5-oxo-4,5-dihydro-3H-pyrazolo[3,4-c]isoquinolin-3-yl) pyrrolidine-1-carbonyl)cyclopropane-1-carbonitrile;
9-(1-((6-chloro-2-(1-methyl-1H-pyrazol-4-yl)pyridin-3-yl)amino)ethyl)-4,7-dimethyl-3-(1-methylpiperidin-4-yl)-3,4-dihydro-5H-pyrazolo[3,4-c]isoquinolin-5-one;
9-[1-[[6-chloro-2-(1-methyl-1,2,4-triazol-3-yl)-3-pyridyl]amino]ethyl]-4,7-dimethyl-3-(1-methyl-4-piperidyl)pyrazolo[3,4-c]isoquinolin-5-one;
9-[1-[[6-chloro-2-(1-ethylpyrazol-4-yl)-3-pyridyl]amino]ethyl]-4,7-dimethyl-3-(1-methyl-4-piperidyl)pyrazolo[3,4-c]isoquinolin-5-one;
9-[1-[[6-chloro-2-(2-methyltetrazol-5-yl)-3-pyridyl]amino]ethyl]-4,7-dimethyl-3-(1-methyl-4-piperidyl)pyrazolo[3,4-c]isoquinolin-5-one;
9-[1-[[6-chloro-2-(1,3,4-oxadiazol-2-yl)-3-pyridyl]amino]ethyl]-4,7-dimethyl-3-(1-methyl-4-piperidyl)pyrazolo[3,4-c]isoquinolin-5-one;
5-[6-chloro-3-[1-[4,7-dimethyl-3-(1-methyl-4-piperidyl)-5-oxo-pyrazolo[3,4-c]isoquinolin-9-yl]ethylamino]-2-pyridyl]-N-methyl-pyridine-2-carboxamide;
3-[6-chloro-3-[1-[4,7-dimethyl-3-(1-methyl-4-piperidyl)-5-oxo-pyrazolo[3,4-c]isoquinolin-9-yl]ethylamino]-2-pyridyl]-4H-1,2,4-oxadiazol-5-one;
9-[1-[[6-chloro-2-(1,3-dimethylpyrazol-4-yl)-3-pyridyl]amino]ethyl]-4,7-dimethyl-3-(1-methyl-4-piperidyl)pyrazolo[3,4-c]isoquinolin-5-one;
9-[1-[[6-chloro-2-(1-methyl-6-oxo-3-pyridyl)-3-pyridyl]amino]ethyl]-4,7-dimethyl-3-(1-methyl-4-piperidyl)pyrazolo[3,4-c]isoquinolin-5-one;
9-[1-[[6-chloro-2-(4-hydroxy-1-piperidyl)-3-pyridyl]amino]ethyl]-4,7-dimethyl-3-(1-methyl-4-piperidyl)pyrazolo[3,4-c]isoquinolin-5-one;
9-[1-[[6-chloro-2-(3-fluoro-4-pyridyl)-3-pyridyl]amino]ethyl]-4,7-dimethyl-3-(1-methyl-4-piperidyl)pyrazolo[3,4-c]isoquinolin-5-one;
9-[1-[[6-chloro-2-(3-pyridyl)-3-pyridyl]amino]ethyl]-4,7-dimethyl-3-(1-methyl-4-piperidyl)pyrazolo[3,4-c]isoquinolin-5-one;
5-[3-[1-[4,7-dimethyl-3-(1-methyl-4-piperidyl)-5-oxo-pyrazolo[3,4-c]isoquinolin-9-yl]ethylamino]-6-methyl-2-pyridyl]-N-methyl-pyridine-2-carboxamide;
9-[1-[[6-chloro-2-(1-methyl-2-oxo-4-pyridyl)-3-pyridyl]amino]ethyl]-4,7-dimethyl-3-(1-methyl-4-piperidyl)pyrazolo[3,4-c]isoquinolin-5-one;
9-[1-[[6-chloro-2-(1-methylpyrazol-4-yl)-3-pyridyl]amino]ethyl]-3-[1-(2-hydroxy-2-methyl-propyl)-4-piperidyl]-4,7-dimethyl-pyrazolo[3,4-c]isoquinolin-5-one;
9-[1-[[6-chloro-2-(1-methylpyrazol-4-yl)-3-pyridyl]amino]ethyl]-3-[1-[(2R)-2-hydroxypropyl]-4-piperidyl]-4,7-dimethyl-pyrazolo[3,4-c]isoquinolin-5-one;
9-[1-[[6-chloro-2-(1-methylpyrazol-4-yl)-3-pyridyl]amino]ethyl]-3-[1-[(2S)-2-hydroxypropyl]-4-piperidyl]-4,7-dimethyl-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2-hydroxy-2-methylpropyl)piperidin-4-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(piperidin-4-yl)-3,4-dihydro-5H-pyrazolo[3,4-c]isoquinolin-5-one;
9-[1-[[6-chloro-2-(1-methyl-1,2,4-triazol-3-yl)-3-pyridyl]amino]ethyl]-3-[1-(2-hydroxyethyl)-4-piperidyl]-4,7-dimethyl-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-((1-methyl-1H-imidazol-2-yl)methyl)piperidin-4-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-(oxetan-3-yl)piperidin-4-yl)-3,4-dihydro-5H-pyrazolo[3,4-c]isoquinolin-5-one;
3-[4-[9-[1-[[6-chloro-2-(1-methyl-1,2,4-triazol-3-yl)-3-pyridyl]amino]ethyl]-4,7-dimethyl-5-oxo-pyrazolo[3,4-c]isoquinolin-3-yl]-1-piperidyl]propanenitrile;
9-(1-((6-chloro-2-(1-methyl-1H-1,2,4-triazol-3-yl)pyridin-3-yl)amino)ethyl)-3-(1-(2,2-difluoroethyl)piperidin-4-yl)-4,7-dimethyl-3,4-dihydro-5H-pyrazolo[3,4-c]isoquinolin-5-one;
3-(1-acetylpiperidin-4-yl)-9-(1-((6-chloro-2-(1-methyl-1H-pyrazol-4-yl)pyridin-3-yl)amino)ethyl)-4,7-dimethyl-3,4-dihydro-5H-pyrazolo[3,4-c]isoquinolin-5-one;
9-[1-[[6-chloro-2-(1-methylpyrazol-4-yl)-3-pyridyl]amino]ethyl]-4,7-dimethyl-3-(1-methylsulfonyl-4-piperidyl)pyrazolo[3,4-c]isoquinolin-5-one;
methyl 4-[9-[1-[[6-chloro-2-(1-methylpyrazol-4-yl)-3-pyridyl]amino]ethyl]-4,7-dimethyl-5-oxo-pyrazolo[3,4-c]isoquinolin-3-yl]piperidine-1-carboxylate;
9-[1-[[6-chloro-2-(1-methylpyrazol-4-yl)-3-pyridyl]amino]ethyl]-3-(1-cyclopropylsulfonyl-4-piperidyl)-4,7-dimethyl-pyrazolo[3,4-c]isoquinolin-5-one;
4-(9-(1-((6-chloro-2-(1-methyl-1H-pyrazol-4-yl)pyridin-3-yl)amino)ethyl)-4,7-dimethyl-5-oxo-4,5-dihydro-3H-pyrazolo[3,4-c]isoquinolin-3-yl)-N,N-dimethylpiperidine-1-carboxamide;
3-(1-acetylpiperidin-4-yl)-9-(1-((6-chloro-1′-methyl-6′-oxo-1′,6′-dihydro-[2,3′-bipyridin]-3-yl)amino)ethyl)-4,7-dimethyl-3,4-dihydro-5H-pyrazolo[3,4-c]isoquinolin-5-one;
3-(1-acetylpiperidin-4-yl)-9-(1-((6-chloro-2-(1-methyl-1H-1,2,4-triazol-3-yl)pyridin-3-yl)amino)ethyl)-4,7-dimethyl-3,4-dihydro-5H-pyrazolo[3,4-c]isoquinolin-5-one;
4,7-dimethyl-9-(1-((6-methyl-2-(1-methyl-1H-pyrazol-4-yl)pyridin-3-yl)amino)ethyl)-3-(1-(methylsulfonyl)piperidin-4-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-(methylsulfonyl)piperidin-4-yl)-3,4-dihydro-5H-pyrazolo[3,4-c]isoquinolin-5-one;
9-(1-((6-chloro-2-(2-methyl-1-oxoisoindolin-5-yl)pyridin-3-yl)amino)ethyl)-4,7-dimethyl-3-(1-(methylsulfonyl)piperidin-4-yl)-3,4-dihydro-5H-pyrazolo[3,4-c]isoquinolin-5-one;
9-(1-((6-chloro-2-(quinolin-6-yl)pyridin-3-yl)amino)ethyl)-4,7-dimethyl-3-(1-(methylsulfonyl)piperidin-4-yl)-3,4-dihydro-5H-pyrazolo[3,4-c]isoquinolin-5-one;
9-[1-[[6-chloro-2-(1-methylpyrazol-4-yl)-3-pyridyl]amino]ethyl]-3-[1-(2-hydroxyacetyl)-4-piperidyl]-4,7-dimethyl-pyrazolo[3,4-c]isoquinolin-5-one;
1-(4-(9-(1-((6-chloro-2-(1-methyl-1H-1,2,4-triazol-3-yl)pyridin-3-yl)amino)ethyl)-4,7-dimethyl-5-oxo-4,5-dihydro-3H-pyrazolo[3,4-c]isoquinolin-3-yl)piperidine-1-carbonyl)cyclopropane-1-carbonitrile;
9-(1-((6-chloro-2-(1-methyl-1H-1,2,4-triazol-3-yl)pyridin-3-yl)amino)ethyl)-4,7-dimethyl-3-(1-(1-methyl-1H-pyrazole-5-carbonyl)piperidin-4-yl)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-(1-(dimethylglycyl)piperidin-4-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-(methyl-L-prolyl)piperidin-4-yl)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-4-methyl-3-(1-methylpiperidin-4-yl)-5-oxo-4,5-dihydro-3H-pyrazolo[3,4-c]isoquinoline-7-carbonitrile;
9-(1-((6-chloro-2-(1-methyl-1H-1,2,4-triazol-3-yl)pyridin-3-yl)amino)ethyl)-4-ethyl-7-methyl-2-(tetrahydro-2H-pyran-4-yl)-2,4-dihydro-5H-pyrazolo[3,4-c]isoquinolin-5-one;
3-(6-chloro-3-((1-(4,7-dimethyl-5-oxo-2-(tetrahydro-2H-pyran-4-yl)-4,5-dihydro-2H-pyrazolo[3,4-c]isoquinolin-9-yl)ethyl)amino)pyridin-2-yl)-1,2,4-oxadiazol-5(4H)-one;
9-(1-((6-chloro-2-(1-methyl-1H-1,2,4-triazol-3-yl)pyridin-3-yl)amino)ethyl)-4-ethyl-7-methyl-2-(1-methylpiperidin-4-yl)-2,4-dihydro-5H-pyrazolo[3,4-c]isoquinolin-5-one;
2-(1-acetylpiperidin-4-yl)-9-(1-((6-chloro-2-(1-methyl-1H-1,2,4-triazol-3-yl)pyridin-3-yl)amino)ethyl)-4-ethyl-7-methyl-2,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4-ethyl-2-(1-(2-hydroxyethyl)piperidin-4-yl)-7-methyl-2,4-dihydro-5H-pyrazolo[3,4-c]isoquinolin-5-one;
3-(4-(9-(1-((6-chloro-2-(1-methyl-1H-1,2,4-triazol-3-yl)pyridin-3-yl)amino)ethyl)-4-ethyl-7-methyl-5-oxo-4,5-dihydro-2H-pyrazolo[3,4-c]isoquinolin-2-yl)piperidin-1-yl)propanenitrile;
9-(1-((6-chloro-2-(1-methyl-1H-1,2,4-triazol-3-yl)pyridin-3-yl)amino)ethyl)-4-ethyl-7-methyl-2-(1-methylazetidin-3-yl)-2,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-4,7-dimethyl-2-(1-methylpiperidin-4-yl)-2,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-2-(1-isopropylpiperidin-4-yl)-4,7-dimethyl-2,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-2-(1-methylpiperidin-4-yl)-2,4-dihydro-5H-pyrazolo[3,4-c]isoquinolin-5-one;
2-(1-acetylpiperidin-4-yl)-9-(1-((6-chloro-2-(1-methyl-1H-pyrazol-4-yl)pyridin-3-yl)amino)ethyl)-4,7-dimethyl-2,4-dihydro-5H-pyrazolo[3,4-c]isoquinolin-5-one;
ethyl 4-(9-(1-((6-chloro-2-(1-methyl-1H-pyrazol-4-yl)pyridin-3-yl)amino)ethyl)-4,7-dimethyl-5-oxo-4,5-dihydro-2H-pyrazolo[3,4-c]isoquinolin-2-yl)piperidine-1-carboxylate;
4-(9-(1-((6-chloro-2-(1-methyl-1H-pyrazol-4-yl)pyridin-3-yl)amino)ethyl)-4,7-dimethyl-5-oxo-4,5-dihydro-2H-pyrazolo[3,4-c]isoquinolin-2-yl)-N,N-dimethylpiperidine-1-carboxamide;
9-(1-((6-chloro-2-(1-methyl-1H-pyrazol-4-yl)pyridin-3-yl)amino)ethyl)-2-(1-(2,2-difluoroethyl)piperidin-4-yl)-4,7-dimethyl-2,4-dihydro-5H-pyrazolo[3,4-c]isoquinolin-5-one;
3-[6-chloro-3-[1-[7-methyl-5-oxo-3-[(3S)-tetrahydrofuran-3-yl]-4-(trideuteriomethyl)pyrazolo[3,4-c]isoquinolin-9-yl]ethylamino]-2-pyridyl]-4H-1,2,4-oxadiazol-5-one;
9-(1-((6-chloro-2-(1-methyl-1H-pyrazol-4-yl)pyridin-3-yl)amino)ethyl)-7-methyl-4-(methyl-d3)-3-((S)-tetrahydrofuran-3-yl)-3,4-dihydro-5H-pyrazolo[3,4-c]isoquinolin-5-one;
3-(6-chloro-3-((1-(4-ethyl-7-methyl-5-oxo-3-((S)-tetrahydrofuran-3-yl)-4,5-dihydro-3H-pyrazolo[3,4-c]isoquinolin-9-yl)ethyl)amino)pyridin-2-yl)-1,2,4-oxadiazol-5(4H)-one;
9-(1-((6-chloro-2-(1-methyl-1H-1,2,4-triazol-3-yl)pyridin-3-yl)amino)ethyl)-4-ethyl-7-methyl-3-((S)-tetrahydrofuran-3-yl)-3,4-dihydro-5H-pyrazolo[3,4-c]isoquinolin-5-one;
9-(1-((6-chloro-2-(1-(1-methylpiperidin-4-yl)-1H-pyrazol-4-yl)pyridin-3-yl)amino)ethyl)-4-ethyl-7-methyl-3-((S)-tetrahydrofuran-3-yl)-3,4-dihydro-5H-pyrazolo[3,4-c]isoquinolin-5-one;
9-(1-((6-chloro-2′-methyl-[2,4′-bipyridin]-3-yl)amino)ethyl)-4-ethyl-7-methyl-3-((S)-tetrahydrofuran-3-yl)-3,4-dihydro-5H-pyrazolo[3,4-c]isoquinolin-5-one;
9-(1-((6-chloro-1′-methyl-6′-oxo-1′,6′-dihydro-[2,3′-bipyridin]-3-yl)amino)ethyl)-4-ethyl-7-methyl-3-((S)-tetrahydrofuran-3-yl)-3,4-dihydro-5H-pyrazolo[3,4-c]isoquinolin-5-one;
6-chloro-3-((1-(4-ethyl-7-methyl-5-oxo-3-((S)-tetrahydrofuran-3-yl)-4,5-dihydro-3H-pyrazolo[3,4-c]isoquinolin-9-yl)ethyl)amino)-N-methyl-[2,3′-bipyridine]-6′-carboxamide;
9-(1-((6-chloro-2-(1-methyl-1H-1,2,4-triazol-3-yl)pyridin-3-yl)amino)ethyl)-4-cyclopropyl-3-ethyl-7-methyl-3,4-dihydro-5H-pyrazolo[3,4-c]isoquinolin-5-one;
9-[1-[[6-chloro-2-(1-methyl-6-oxo-3-pyridyl)-3-pyridyl]amino]ethyl]-4-cyclopropyl-3-ethyl-7-methyl-pyrazolo[3,4-c]isoquinolin-5-one;
9-[1-[[6-chloro-2-(1-methyl-1,2,4-triazol-3-yl)-3-pyridyl]amino]ethyl]-3-ethyl-7-methyl-4-tetrahydrofuran-3-yl-pyrazolo[3,4-c]isoquinolin-5-one;
9-[1-[[6-chloro-2-(1-methyl-1,2,4-triazol-3-yl)-3-pyridyl]amino]ethyl]-3-ethyl-4-(2-hydroxyethyl)-7-methyl-pyrazolo[3,4-c]isoquinolin-5-one;
4-(6-chloro-3-(((3-ethyl-4,7-dimethyl-5-oxo-4,5-dihydro-3H-pyrazolo[3,4-c]isoquinolin-9-yl)methyl)amino)pyridin-2-yl)-2-fluoro-N-methylbenzamide;
9-(1-((6-chloro-2-(3-methyl-1H-pyrazol-4-yl)pyridin-3-yl)oxy)ethyl)-3-ethyl-4,7-dimethyl-3,4-dihydro-5H-pyrazolo[3,4-c]isoquinolin-5-one;
3-(6-chloro-3-((1-(3-ethyl-7-methyl-4-(methyl-d3)-5-oxo-4,5-dihydro-3H-pyrazolo[3,4-c]isoquinolin-9-yl)ethyl)amino)pyridin-2-yl)-1,2,4-oxadiazol-5(4H)-one;
9-(1-((6-chloro-2-(1-methyl-1H-1,2,4-triazol-3-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(4-methyl-4H-1,2,4-triazol-3-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(1H-tetrazol-5-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(1-methyl-1H-1,2,3-triazol-5-yl)pyridin-3-yl)amino)ethyl)-3-ethyl-4,7-dimethyl-3,4-dihydro-5H-pyrazolo[3,4-c]isoquinolin-5-one;
4-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)benzoic acid;
4-(4-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)-1H-pyrazol-1-yl)-N,N-dimethylpiperidine-1-carboxamide;
3-(4-(4-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)-1H-pyrazol-1-yl)piperidin-1-yl)propanenitrile;
3-ethyl-4,7-dimethyl-9-(1-((6-methyl-2-(3-methyl-1H-pyrazol-4-yl)pyridin-3-yl)amino)ethyl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4-(2,2-difluoroethyl)-3-ethyl-7-methyl-3,4-dihydro-5H-pyrazolo[3,4-c]isoquinolin-5-one;
9-(1-((6-chloro-2′-methyl-[2,4′-bipyridin]-3-yl)amino)ethyl)-4-(2,2-difluoroethyl)-3-ethyl-7-methyl-3,4-dihydro-5H-pyrazolo[3,4-c]isoquinolin-5-one;
9-(1-((6-chloro-2-(pyrimidin-5-yl)pyridin-3-yl)amino)ethyl)-4-(2,2-difluoroethyl)-3-ethyl-7-methyl-3,4-dihydro-5H-pyrazolo[3,4-c]isoquinolin-5-one;
9-(1-((6-chloro-1′-methyl-6′-oxo-1′,6′-dihydro-[2,3′-bipyridin]-3-yl)amino)ethyl)-4-(2,2-difluoroethyl)-3-ethyl-7-methyl-3,4-dihydro-5H-pyrazolo[3,4-c]isoquinolin-5-one;
6-chloro-3-((1-(4-(2,2-difluoroethyl)-3-ethyl-7-methyl-5-oxo-4,5-dihydro-3H-pyrazolo[3,4-c]isoquinolin-9-yl)ethyl)amino)-N-methyl-[2,3′-bipyridine]-6′-carboxamide;
9-[1-[[2-(1-acetyl-4-piperidyl)-6-chloro-3-pyridyl]amino]ethyl]-3-ethyl-4,7-dimethyl-pyrazolo[3,4-c]isoquinolin-5-one;
9-(1-((6-chloro-2-(1-methyl-1H-1,2,4-triazol-3-yl)pyridin-3-yl)amino)ethyl)-3-(2-hydroxyethyl)-4,7-dimethyl-3,4-dihydro-5H-pyrazolo[3,4-c]isoquinolin-5-one;
4-(6-chloro-3-((1-(3-(2-hydroxyethyl)-4,7-dimethyl-5-oxo-4,5-dihydro-3H-pyrazolo[3,4-c]isoquinolin-9-yl)ethyl)amino)pyridin-2-yl)-2-fluoro-N-methylbenzamide;
9-(1-((6-chloro-2-(1-methyl-1H-pyrazol-4-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(4-hydroxypiperidin-1-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(quinolin-6-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(1-methyl-1H-indazol-5-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(2-methyl-1-oxoisoindolin-5-yl)pyridin-3-yl)amino)ethyl)-3-(2-hydroxyethyl)-4,7-dimethyl-3,4-dihydro-5H-pyrazolo[3,4-c]isoquinolin-5-one;
3-(2-hydroxyethyl)-4,7-dimethyl-9-(1-((6-methyl-2-(1-methyl-1H-pyrazol-4-yl)pyridin-3-yl)amino)ethyl)-3,4-dihydro-5H-pyrazolo[3,4-c]isoquinolin-5-one;
9-(1-((6-chloro-2-(1,3,4-oxadiazol-2-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(2-methyl-2H-tetrazol-5-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(pyrimidin-5-yl)pyridin-3-yl)amino)ethyl)-3-(2-hydroxyethyl)-4,7-dimethyl-3,4-dihydro-5H-pyrazolo[3,4-c]isoquinolin-5-one;
5-[6-chloro-3-[1-[3-(2-hydroxyethyl)-4,7-dimethyl-5-oxo-pyrazolo[3,4-c]isoquinolin-9-yl]ethylamino]-2-pyridyl]-N-methyl-pyridine-2-carboxamide;
9-[1-[[6-chloro-2-(1-methyl-6-oxo-3-pyridyl)-3-pyridyl]amino]ethyl]-3-(2-hydroxyethyl)-4,7-dimethyl-pyrazolo[3,4-c]isoquinolin-5-one;
9-[1-[[6-chloro-2-(1-methyl-2-oxo-4-pyridyl)-3-pyridyl]amino]ethyl]-3-(2-hydroxyethyl)-4,7-dimethyl-pyrazolo[3,4-c]isoquinolin-5-one;
9-[1-[[6-chloro-2-(2-methoxy-4-pyridyl)-3-pyridyl]amino]ethyl]-3-(2-hydroxyethyl)-4,7-dimethyl-pyrazolo[3,4-c]isoquinolin-5-one;
9-[1-[[6-chloro-2-(1-methyl-4-piperidyl)-3-pyridyl]amino]ethyl]-3-(2-hydroxyethyl)-4,7-dimethyl-pyrazolo[3,4-c]isoquinolin-5-one;
9-[1-[[2-(1-acetyl-4-piperidyl)-6-chloro-3-pyridyl]amino]ethyl]-3-(2-hydroxyethyl)-4,7-dimethyl-pyrazolo[3,4-c]isoquinolin-5-one;
9-[1-[[6-chloro-2-(1-methyl-1,2,4-triazol-3-yl)-3-pyridyl]amino]ethyl]-4-(2,2-difluoroethyl)-3-(2-hydroxyethyl)-7-methyl-pyrazolo[3,4-c]isoquinolin-5-one;
5-[6-chloro-3-[1-[4-(2,2-difluoroethyl)-3-(2-hydroxyethyl)-7-methyl-5-oxo-pyrazolo[3,4-c]isoquinolin-9-yl]ethylamino]-2-pyridyl]-N-methyl-pyridine-2-carboxamide;
9-[1-[[6-chloro-2-(1-methyl-1,2,4-triazol-3-yl)-3-pyridyl]amino]ethyl]-4-ethyl-3-(2-hydroxyethyl)-7-methyl-pyrazolo[3,4-c]isoquinolin-5-one;
9-[1-[(6-chloro-2-morpholino-3-pyridyl)amino]ethyl]-4-ethyl-3-(2-hydroxyethyl)-7-methyl-pyrazolo[3,4-c]isoquinolin-5-one;
9-[1-[[6-chloro-2-(1-methylpyrazol-4-yl)-3-pyridyl]amino]ethyl]-4,7-dimethyl-3-(2-morpholinoethyl)pyrazolo[3,4-c]isoquinolin-5-one;
9-(1-((6-chloro-3′-fluoro-[2,4′-bipyridin]-3-yl)amino)ethyl)-4,7-dimethyl-3-(2-morpholinoethyl)-3,4-dihydro-5H-pyrazolo[3,4-c]isoquinolin-5-one;
9-(1-((6-chloro-3′-fluoro-[2,4′-bipyridin]-3-yl)amino)ethyl)-4,7-dimethyl-3-(2-(4-methylpiperazin-1-yl)ethyl)-3,4-dihydro-5H-pyrazolo[3,4-c]isoquinolin-5-one;
4-(6-chloro-3-((1-(4,7-dimethyl-3-(2-morpholinoethyl)-5-oxo-4,5-dihydro-3H-pyrazolo[3,4-c]isoquinolin-9-yl)ethyl)amino)pyridin-2-yl)-2-fluoro-N-methylbenzamide;
4-(6-chloro-3-((1-(4,7-dimethyl-3-(2-(4-methylpiperazin-1-yl)ethyl)-5-oxo-4,5-dihydro-3H-pyrazolo[3,4-c]isoquinolin-9-yl)ethyl)amino)pyridin-2-yl)-2-fluoro-N-methylbenzamide;
9-(1-((6-chloro-2-(1-methyl-1H-1,2,4-triazol-3-yl)pyridin-3-yl)amino)ethyl)-4-(2,2-difluoroethyl)-3-(2-(5,6-dihydro-[1,2,4]triazolo[1,5-a]pyrazin-7(8H)-yl)ethyl)-7-methyl-3,4-dihydro-5H-pyrazolo[3,4-c]isoquinolin-5-one;
9-[1-[[6-chloro-2-(1-methyl-1,2,4-triazol-3-yl)-3-pyridyl]amino]ethyl]-4-ethyl-7-methyl-3-(2-pyridyl)pyrazolo[3,4-c]isoquinolin-5-one;
9-(1-((6-chloro-2-morpholinopyridin-3-yl)amino)ethyl)-4-ethyl-7-methyl-3-(pyridin-2-yl)-3,4-dihydro-5H-pyrazolo[3,4-c]isoquinolin-5-one;
6-chloro-3-((1-(4-ethyl-7-methyl-5-oxo-3-(pyridin-2-yl)-4,5-dihydro-3H-pyrazolo[3,4-c]isoquinolin-9-yl)ethyl)amino)-N-methyl-[2,3′-bipyridine]-6′-carboxamide;
3-(6-chloro-3-((1-(4,7-dimethyl-5-oxo-3-(pyridin-2-yl)-4,5-dihydro-3H-pyrazolo[3,4-c]isoquinolin-9-yl)ethyl)amino)pyridin-2-yl)-1,2,4-oxadiazol-5(4H)-one;
9-(1-((6-chloro-2-(1-methyl-1H-pyrazol-4-yl)pyridin-3-yl)amino)ethyl)-4,7-dimethyl-3-(pyridin-2-yl)-3,4-dihydro-5H-pyrazolo[3,4-c]isoquinolin-5-one;
9-[1-[[6-chloro-2-(1-methyl-1,2,4-triazol-3-yl)-3-pyridyl]amino]ethyl]-4-ethyl-7-methyl-3-(3-pyridyl)pyrazolo[3,4-c]isoquinolin-5-one;
9-[1-[[6-chloro-2-(1-methylpyrazol-4-yl)-3-pyridyl]amino]ethyl]-4,7-dimethyl-3-(2-pyridylmethyl)pyrazolo[3,4-c]isoquinolin-5-one;
3-(6-chloro-3-((1-(4,7-dimethyl-5-oxo-3-(pyridin-2-ylmethyl)-4,5-dihydro-3H-pyrazolo[3,4-c]isoquinolin-9-yl)ethyl)amino)pyridin-2-yl)-1,2,4-oxadiazol-5(4H)-one;
9-[1-[[6-chloro-2-(1-methyl-1,2,4-triazol-3-yl)-3-pyridyl]amino]ethyl]-4,7-dimethyl-3-(1-methyl-3-piperidyl)pyrazolo[3,4-c]isoquinolin-5-one;
9-[1-[[6-chloro-2-(1-methyl-1,2,4-triazol-3-yl)-3-pyridyl]amino]ethyl]-3-[1-(2-hydroxyethyl)-3-piperidyl]-4,7-dimethyl-pyrazolo[3,4-c]isoquinolin-5-one;
9-[1-[[6-chloro-2-(1-methylpyrazol-4-yl)-3-pyridyl]amino]ethyl]-4,7-dimethyl-3-(1-methyl-3-piperidyl)pyrazolo[3,4-c]isoquinolin-5-one;
9-[1-[[6-chloro-2-(1-methylpyrazol-4-yl)-3-pyridyl]amino]ethyl]-3-[1-(2-hydroxyethyl)-3-piperidyl]-4,7-dimethyl-pyrazolo[3,4-c]isoquinolin-5-one;
methyl 3-[9-[1-[[6-chloro-2-(1-methylpyrazol-4-yl)-3-pyridyl]amino]ethyl]-4,7-dimethyl-5-oxo-pyrazolo[3,4-c]isoquinolin-3-yl]piperidine-1-carboxylate;
5-[6-chloro-3-[1-[4,7-dimethyl-3-(1-methyl-4-piperidyl)-5-oxo-pyrazolo[3,4-c]isoquinolin-9-yl]ethoxy]-2-pyridyl]-N-methyl-pyridine-2-carboxamide;
5-[3-[1-[3-(1-acetyl-4-piperidyl)-4,7-dimethyl-5-oxo-pyrazolo[3,4-c]isoquinolin-9-yl]ethoxy]-6-chloro-2-pyridyl]-N-methyl-pyridine-2-carboxamide;
10-(1-((6-chloro-2-(1-methyl-1H-1,2,4-triazol-3-yl)pyridin-3-yl)amino)ethyl)-3-(hydroxymethyl)-8-methyl-4,5-dihydro-3H,6H-2,2a,5a-triazaaceanthrylen-6-one;
10-(1-((6-chloro-2-(2-methyl-2H-tetrazol-5-yl)pyridin-3-yl)amino)ethyl)-3-(hydroxymethyl)-8-methyl-4,5-dihydro-3H,6H-2,2a,5a-triazaaceanthrylen-6-one;
10-(1-((6-chloro-2-(1,3,4-oxadiazol-2-yl)pyridin-3-yl)amino)ethyl)-3-(hydroxymethyl)-8-methyl-4,5-dihydro-3H,6H-2,2a,5a-triazaaceanthrylen-6-one;
10-(1-((6-chloro-2-(1-methyl-1H-pyrazol-4-yl)pyridin-3-yl)amino)ethyl)-3-(hydroxymethyl)-8-methyl-4,5-dihydro-3H,6H-2,2a,5a-triazaaceanthrylen-6-one;
10-(1-((6-chloro-2-(3-methyl-1H-pyrazol-4-yl)pyridin-3-yl)amino)ethyl)-3-(hydroxymethyl)-8-methyl-4,5-dihydro-3H,6H-2,2a,5a-triazaaceanthrylen-6-one;
10-(1-((6-chloro-1′-methyl-6′-oxo-1′,6′-dihydro-[2,3′-bipyridin]-3-yl)amino)ethyl)-3-(hydroxymethyl)-8-methyl-4,5-dihydro-3H,6H-2,2a,5a-triazaaceanthrylen-6-one;
10-(1-((6-chloro-2-(1-methyl-1H-1,2,4-triazol-3-yl)pyridin-3-yl)amino)ethyl)-3-((dimethylamino)methyl)-8-methyl-4,5-dihydro-3H,6H-2,2a,5a-triazaaceanthrylen-6-one;
10-(1-((6-chloro-2-(4-hydroxypiperidin-1-yl)pyridin-3-yl)amino)ethyl)-8-methyl-4,5-dihydro-3H,6H-2,2a,5a-triazaaceanthrylen-6-one;
1-(6-chloro-3-((1-(8-methyl-6-oxo-4,5-dihydro-3H,6H-2,2a,5a-triazaaceanthrylen-10-yl)ethyl)amino)pyridin-2-yl)-N-methylpiperidine-4-carboxamide;
10-(1-((2-(4-acetylpiperazin-1-yl)-6-chloropyridin-3-yl)amino)ethyl)-8-methyl-4,5-dihydro-3H,6H-2,2a,5a-triazaaceanthrylen-6-one;
10-(1-((6-chloro-2-(1,3,4-oxadiazol-2-yl)pyridin-3-yl)amino)ethyl)-8-methyl-4,5-dihydro-3H,6H-2,2a,5a-triazaaceanthrylen-6-one;
3-(6-chloro-3-((1-(8-methyl-6-oxo-4,5-dihydro-3H,6H-2,2a,5a-triazaaceanthrylen-10-yl)ethyl)amino)pyridin-2-yl)-1,2,4-oxadiazol-5(4H)-one;
10-(1-((6-chloro-2-(1-methyl-1H-1,2,4-triazol-3-yl)pyridin-3-yl)amino)ethyl)-8-methyl-4,5-dihydro-3H,6H-2,2a,5a-triazaaceanthrylen-6-one;
10-(1-((6-chloro-2-(1-(2-hydroxyethyl)-1H-1,2,4-triazol-3-yl)pyridin-3-yl)amino)ethyl)-8-methyl-4,5-dihydro-3H,6H-2,2a,5a-triazaaceanthrylen-6-one;
10-(1-((6-chloro-2-(2-methyl-2H-tetrazol-5-yl)pyridin-3-yl)amino)ethyl)-8-methyl-4,5-dihydro-3H,6H-2,2a,5a-triazaaceanthrylen-6-one;
10-(1-((6-chloro-2-(1-(tetrahydro-2H-pyran-4-yl)-1H-pyrazol-4-yl)pyridin-3-yl)amino)ethyl)-8-methyl-4,5-dihydro-3H,6H-2,2a,5a-triazaaceanthrylen-6-one;
10-(1-((6-chloro-2-(1-(2-hydroxyethyl)-1H-pyrazol-4-yl)pyridin-3-yl)amino)ethyl)-8-methyl-4,5-dihydro-3H,6H-2,2a,5a-triazaaceanthrylen-6-one;
10-(1-((6-chloro-2-(1-(1-methylpiperidin-4-yl)-1H-pyrazol-4-yl)pyridin-3-yl)amino)ethyl)-8-methyl-4,5-dihydro-3H,6H-2,2a,5a-triazaaceanthrylen-6-one;
10-(1-((6-chloro-2-(1-(1-methylpiperidin-3-yl)-1H-pyrazol-4-yl)pyridin-3-yl)amino)ethyl)-8-methyl-4,5-dihydro-3H,6H-2,2a,5a-triazaaceanthrylen-6-one;
10-(1-((6-chloro-2-(1-(1-methylpyrrolidin-3-yl)-1H-pyrazol-4-yl)pyridin-3-yl)amino)ethyl)-8-methyl-4,5-dihydro-3H,6H-2,2a,5a-triazaaceanthrylen-6-one;
10-(1-((6-chloro-2-(1-(1-methylazetidin-3-yl)-1H-pyrazol-4-yl)pyridin-3-yl)amino)ethyl)-8-methyl-4,5-dihydro-3H,6H-2,2a,5a-triazaaceanthrylen-6-one;
4-(4-(6-chloro-3-((1-(8-methyl-6-oxo-4,5-dihydro-3H,6H-2,2a,5a-triazaaceanthrylen-10-yl)ethyl)amino)pyridin-2-yl)-1H-pyrazol-1-yl)-N,N-dimethylpiperidine-1-carboxamide;
10-(1-((6-chloro-2′-methyl-[2,4′-bipyridin]-3-yl)amino)ethyl)-8-methyl-4,5-dihydro-3H,6H-2,2a,5a-triazaaceanthrylen-6-one;
10-(1-((6-chloro-[2,3′-bipyridin]-3-yl)amino)ethyl)-8-methyl-4,5-dihydro-3H,6H-2,2a,5a-triazaaceanthrylen-6-one;
10-(1-((6-chloro-1′-methyl-6′-oxo-1′,6′-dihydro-[2,3′-bipyridin]-3-yl)amino)ethyl)-8-methyl-4,5-dihydro-3H,6H-2,2a,5a-triazaaceanthrylen-6-one;
4-(6-chloro-3-((1-(8-methyl-6-oxo-4,5-dihydro-3H,6H-2,2a,5a-triazaaceanthrylen-10-yl)ethyl)amino)pyridin-2-yl)-2-fluoro-N-methylbenzamide;
6-chloro-N-methyl-3-((1-(8-methyl-6-oxo-4,5-dihydro-3H,6H-2,2a,5a-triazaaceanthrylen-10-yl)ethyl)amino)-[2,3′-bipyridine]-6′-carboxamide;
10-(1-((6-chloro-6′-methoxy-[2,3′-bipyridin]-3-yl)amino)ethyl)-8-methyl-4,5-dihydro-3H,6H-2,2a,5a-triazaaceanthrylen-6-one;
10-(1-((6-chloro-2′-methoxy-[2,4′-bipyridin]-3-yl)amino)ethyl)-8-methyl-4,5-dihydro-3H,6H-2,2a,5a-triazaaceanthrylen-6-one;
10-(1-((6-chloro-2′-(dimethylamino)-[2,4′-bipyridin]-3-yl)amino)ethyl)-8-methyl-4,5-dihydro-3H,6H-2,2a,5a-triazaaceanthrylen-6-one;
10-(1-((6-chloro-2-(2-methyl-1-oxoisoindolin-5-yl)pyridin-3-yl)amino)ethyl)-8-methyl-4,5-dihydro-3H,6H-2,2a,5a-triazaaceanthrylen-6-one;
10-(1-((6-chloro-2-(quinolin-6-yl)pyridin-3-yl)amino)ethyl)-8-methyl-4,5-dihydro-3H,6H-2,2a,5a-triazaaceanthrylen-6-one;
10-(1-((6-chloro-2-(1-methyl-1H-indazol-5-yl)pyridin-3-yl)amino)ethyl)-8-methyl-4,5-dihydro-3H,6H-2,2a,5a-triazaaceanthrylen-6-one;
2-fluoro-N-methyl-4-(6-methyl-3-((1-(8-methyl-6-oxo-4,5-dihydro-3H,6H-2,2a,5a-triazaaceanthrylen-10-yl)ethyl)amino)pyridin-2-yl)benzamide;
8-methyl-10-(1-((6-methyl-2-(1-(tetrahydro-2H-pyran-4-yl)-1H-pyrazol-4-yl)pyridin-3-yl)amino)ethyl)-4,5-dihydro-3H,6H-2,2a,5a-triazaaceanthrylen-6-one;
10-(1-((2-(1-(2-hydroxyethyl)-1H-pyrazol-4-yl)-6-methylpyridin-3-yl)amino)ethyl)-8-methyl-4,5-dihydro-3H,6H-2,2a,5a-triazaaceanthrylen-6-one;
10-(1-((2′,6-dimethyl-[2,4′-bipyridin]-3-yl)amino)ethyl)-8-methyl-4,5-dihydro-3H,6H-2,2a,5a-triazaaceanthrylen-6-one;
10-(1-((2-(1-cyclopropyl-1H-pyrazol-4-yl)-6-methylpyridin-3-yl)amino)ethyl)-8-methyl-4,5-dihydro-3H,6H-2,2a,5a-triazaaceanthrylen-6-one;
7-(6-chloro-3-((1-(8-methyl-6-oxo-4,5-dihydro-3H,6H-2,2a,5a-triazaaceanthrylen-10-yl)ethyl)amino)pyridin-2-yl) hexahydro-3H-oxazolo[3,4-a]pyrazin-3-one;
10-(1-((6-chloro-2-(5-methyl-4,5,6,7-tetrahydropyrazolo[1,5-a]pyrazin-3-yl)pyridin-3-yl)amino)ethyl)-8-methyl-4,5-dihydro-3H,6H-2,2a,5a-triazaaceanthrylen-6-one;
10-(1-((6-chloro-6′-((dimethylamino)methyl)-[2,3′-bipyridin]-3-yl)amino)ethyl)-8-methyl-4,5-dihydro-3H,6H-2,2a,5a-triazaaceanthrylen-6-one;
3-(3-((1-(3,8-dimethyl-6-oxo-4,5-dihydro-3H,6H-2,2a,5a-triazaaceanthrylen-10-yl)ethyl)amino)-6-methylpyridin-2-yl)-1,2,4-oxadiazol-5(4H)-one;
8-methyl-10-(1-((6-methyl-2-(2-methyl-2H-tetrazol-5-yl)pyridin-3-yl)amino)ethyl)-4,5-dihydro-3H,6H-2,2a,5a-triazaaceanthrylen-6-one;
3-(3-((1-(3-ethyl-4,7-dimethyl-5-oxo-4,5-dihydro-3H-pyrazolo[3,4-c]isoquinolin-9-yl)ethyl)amino)-6-methylpyridin-2-yl)-1,2,4-oxadiazol-5(4H)-one;
10-(1-((6-chloro-2-(1-methyl-1H-1,2,4-triazol-3-yl)pyridin-3-yl)amino)-2-hydroxyethyl)-8-methyl-4,5-dihydro-3H,6H-2,2a,5a-triazaaceanthrylen-6-one;
10-(1-((6-chloro-2-(1-methyl-1H-pyrazol-4-yl)pyridin-3-yl)amino)-2-hydroxyethyl)-8-methyl-4,5-dihydro-3H,6H-2,2a,5a-triazaaceanthrylen-6-one;
10-(1-((6-chloro-2-(1,5-dimethyl-1H-pyrazol-4-yl)pyridin-3-yl)amino)-2-hydroxyethyl)-8-methyl-4,5-dihydro-3H,6H-2,2a,5a-triazaaceanthrylen-6-one;
10-(1-((6-chloro-2-(1,3,4-oxadiazol-2-yl)pyridin-3-yl)amino)-2-hydroxyethyl)-8-methyl-4,5-dihydro-3H,6H-2,2a,5a-triazaaceanthrylen-6-one;
10-(1-((6-chloro-2-(2-methyl-2H-tetrazol-5-yl)pyridin-3-yl)amino)-2-hydroxyethyl)-8-methyl-4,5-dihydro-3H,6H-2,2a,5a-triazaaceanthrylen-6-one;
10-(1-((6-chloro-2-(2-methyl-1-oxoisoindolin-5-yl)pyridin-3-yl)amino)-2-hydroxyethyl)-8-methyl-4,5-dihydro-3H,6H-2,2a,5a-triazaaceanthrylen-6-one; and
9-(1-((6-chloro-2-(1-methyl-1H-pyrazol-4-yl)pyridin-3-yl)amino)-2-(4-methylpiperazin-1-yl)ethyl)-3-ethyl-4,7-dimethyl-3,4-dihydro-5H-pyrazolo[3,4-c]isoquinolin-5-one;
   or a pharmaceutically acceptable salt thereof.

In some embodiments, the present disclosure encompasses the recognition that provided compounds display certain desirable characteristics, e.g., as compared to other known compounds. For example, in some embodiments, provided compounds are more potent in one or more biochemical or cellular assays described herein, and/or have one or more other characteristics that make them more suitable for drug development, such as better selectivity for mutant PI3Kα over wide-type (WT) PI3Kα and/or better ADME (absorption, distribution, metabolism, and excretion) properties including but not limited to better permeability, cytotoxicity, hepatocyte stability, solubility, and/or plasma protein binding profiles, than other known compounds. In some embodiments, provided compounds display certain desirable characteristics in one or more assays described herein, e.g., compared to other known compounds.

In some embodiments, provided compounds are provided and/or utilized in a salt form (e.g., a pharmaceutically acceptable salt form). Reference to a compound provided herein is understood to include reference to salts thereof, unless otherwise indicated.

It will be understood that, unless otherwise specified or prohibited by the foregoing definition of any of Formulae I-VIIb, embodiments of variables X, Y, Z, U, Ring A, L^{A}, R^{A}, R^{A1}, R^{AG}, R¹, R², R³, R⁴, R⁵, L⁵, R^{5A}, R^{5A1}, R^{5A11}, R^{5A12}, R^{5AG}, R^{G1}, R, R^{m}, and n, as defined above and described in classes and subclasses herein, apply to compounds of any of Formulae I-VIIb, both singly and in combination.

It will be understood that, unless otherwise specified or prohibited by the foregoing definition of any of Formulae I, II, and III, embodiments of variables X, Y, Z, U, Ring A, L^{A}, R^{A}, R^{A1}, R^{AG}, R¹, R², R³, R⁴, R⁵, L⁵, R^{5A}, R^{5A1}, R^{5A11}, R^{5A12}, R^{5AG}, R^{G1}, R, R^{m}, and n, as defined above and described in classes and subclasses herein, apply to compounds of any of Formulae I, II, and III, both singly and in combination.

It will be appreciated that throughout the present disclosure, unless otherwise indicated, reference to a compound of Formula I is intended to also include any of Formulae I, II, and III, and compound species of such formulae disclosed herein.

### Preparing Provided Compounds

Compounds of the present disclosure, including salts thereof, can be prepared using known organic synthesis techniques and can be synthesized according to numerous possible synthetic routes, such as those in the schemes below. The schemes below provide general guidance in connection with preparing the compounds of the present disclosure. One skilled in the art would understand that the preparations shown in the schemes can be modified or optimized using general knowledge of organic chemistry to prepare various compounds of the present disclosure. Provided compounds may generally be made by the processes described in the ensuing schemes and examples.

In some embodiments, provided compounds are prepared according to the following Scheme:

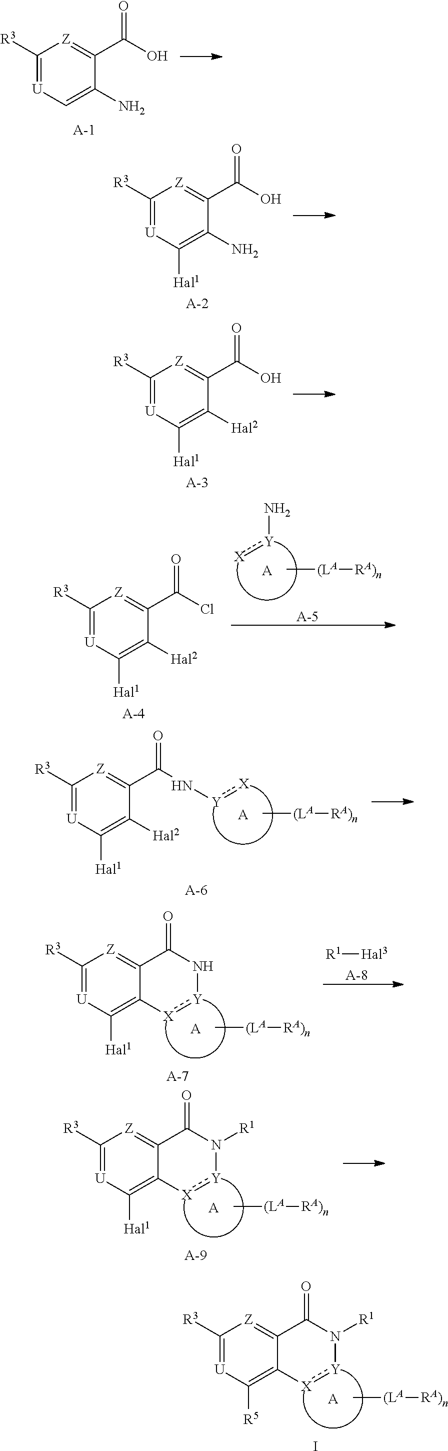

wherein each of Hal¹, Hal², and Hal³ is independently suitable halogen, and each of X, Y, Z, U, R¹, R³, R⁵, Ring A, L^{A}, R^{A}, and n is as defined above for Formula I, and described in classes and subclasses herein, both singly and in combination. Accordingly, in some embodiments, intermediate A-2 is prepared by a process comprising reacting compounds of Formula A-1 with a suitable reagent (e.g., N-bromosuccinimide). In some embodiments, intermediate A-3 is prepared by a process comprising reacting intermediate A-2 under Sandmeyer reaction conditions. In some embodiments, intermediate A-4 is prepared by a process comprising reacting intermediate A-3 with a suitable reagent (e.g., oxalyl chloride or thionyl chloride). In some embodiments, intermediate A-6 is prepared by a process comprising contacting intermediate A-4 with compounds of Formula A-5 under suitable conditions. In some embodiments, intermediate A-7 is prepared by a process comprising reacting intermediate A-6 under suitable cyclization conditions (e.g., S_{N}Ar reaction or C—H activation). In some embodiments, intermediate A-9 is prepared by a process comprising reacting intermediate A-7 with compounds of Formula A-8. In some embodiments, compounds of Formula I are prepared by a process comprising reacting intermediate A-9 under suitable conditions (e.g., transition metal-catalyzed cross-coupling reactions).

In some embodiments, provided compounds are prepared according to the following Scheme:

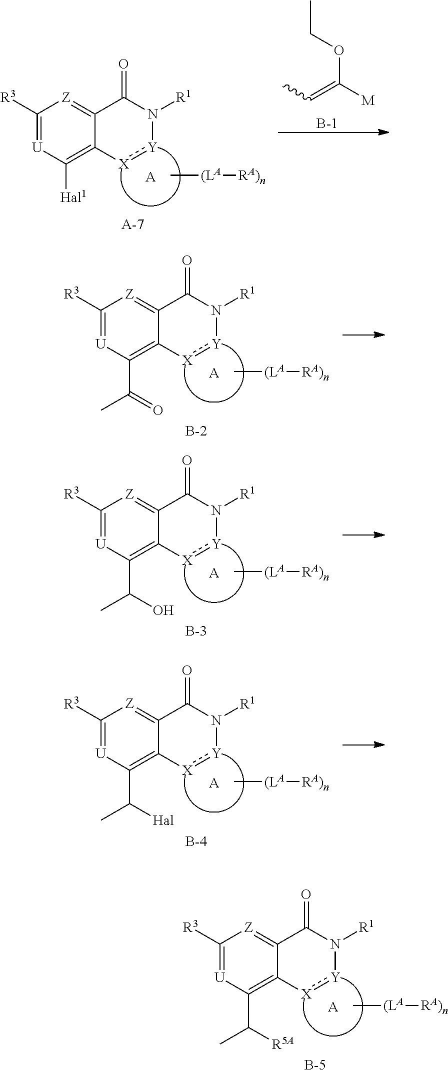

wherein each of Hal and Hal¹ is a suitable halogen, M is a suitable reacting group (e.g., pinacol boronic ester group or tributyltin group), and each of X, Y, Z, U, R¹, R³, R^{5A}, Ring A, L^{A}, R^{A} and n is as defined above for Formula I, and described in classes and subclasses herein, both singly and in combination. Accordingly, in some embodiments, intermediate B-2 is prepared by a process comprising contacting compounds of Formula A-7 and B-1 under suitable conditions (e.g., metal-catalyzed cross-coupling reactions). In some embodiments, intermediate B-3 is prepared by a process comprising reacting intermediate B-2 under suitable reducing conditions. In some embodiments, intermediate B-4 is prepared by a process comprising reacting intermediate B-3 with a suitable reagent (e.g., PBr₃). In some embodiments, compounds of Formula B-5 are prepared by a process comprising reacting intermediate B-4 with suitable nucleophiles.

### Compositions

The present disclosure also provides compositions comprising a compound provided herein with one or more other components. In some embodiments, provided compositions comprise and/or deliver a compound described herein (e.g., compounds of any of Formulae I, II, and III).

In some embodiments, a provided composition is a pharmaceutical composition that comprises and/or delivers a compound provided herein (e.g., compounds of any of Formulae I, II, and III) and further comprises a pharmaceutically acceptable carrier.

Pharmaceutical compositions typically contain an active agent (e.g., a compound described herein) in an amount effective to achieve a desired therapeutic effect while avoiding or minimizing adverse side effects. In some embodiments, provided pharmaceutical compositions comprise a compound described herein and one or more fillers, disintegrants, lubricants, glidants, anti-adherents, and/or anti-statics, etc. Provided pharmaceutical compositions can be in a variety of forms including oral dosage forms, topical creams, topical patches, iontophoresis forms, suppository, nasal spray and/or inhaler, eye drops, intraocular injection forms, depot forms, as well as injectable and infusible solutions. Methods of preparing pharmaceutical compositions are well known in the art.

In some embodiments, provided compounds are formulated in a unit dosage form for ease of administration and uniformity of dosage. The expression “unit dosage form” as used herein refers to a physically discrete unit of an active agent (e.g., a compound described herein) for administration to a subject. Typically, each such unit contains a predetermined quantity of active agent. In some embodiments, a unit dosage form contains an entire single dose of the agent. In some embodiments, more than one unit dosage form is administered to achieve a total single dose. In some embodiments, administration of multiple unit dosage forms is required, or expected to be required, in order to achieve an intended effect. A unit dosage form may be, for example, a liquid pharmaceutical composition containing a predetermined quantity of one or more active agents, a solid pharmaceutical composition (e.g., a tablet, a capsule, or the like) containing a predetermined amount of one or more active agents, a sustained release formulation containing a predetermined quantity of one or more active agents, or a drug delivery device containing a predetermined amount of one or more active agents, etc.

Provided compositions may be administered using any amount and any route of administration effective for treating or lessening the severity of any disease or disorder described herein.

### Uses

The present disclosure provides uses for compounds and compositions described herein. In some embodiments, provided compounds and compositions are for use in medicine (e.g., as therapy). In some embodiments, provided compounds and compositions are useful in treating a disease, disorder, or condition, wherein an underlying pathology is, wholly or partially, mediated by PI3Kα. In some embodiments, provided compounds and compositions are useful in research as, for example, analytical tools and/or control compounds in biological assays.

In some embodiments, the present disclosure provides methods of administering provided compounds or compositions to a subject in need thereof. In some embodiments, the present disclosure provides methods of administering provided compounds or compositions to a subject suffering from or susceptible to a disease, disorder, or condition associated with PI3Kα. In some embodiments, the present disclosure provides methods of administering provided compounds or compositions to a subject suffering from or susceptible to a disease, disorder, or condition, wherein an underlying pathology is, wholly or partially, mediated by PI3Kα.

In some embodiments, provided compounds are useful as PI3Kα inhibitors. In some embodiments, the present disclosure provides methods of inhibiting PI3Kα in a subject comprising administering a provided compound or composition. In some embodiments, the present disclosure provides methods of inhibiting PI3Kα in a biological sample comprising contacting the sample with a provided compound or composition.

In some embodiments, the present disclosure provides methods of treating a disease, disorder or condition associated with PI3Kα in a subject in need thereof, comprising administering to the subject a provided compound or composition. In some embodiments, a disease, disorder or condition is associated with mutation of PI3Kα. In some embodiments, the present disclosure provides methods of treating a disease, disorder or condition, wherein an underlying pathology is, wholly or partially, mediated by PI3Kα, in a subject in need thereof, comprising administering to the subject a provided compound or composition.

In some embodiments, the present disclosure provides methods of treating a variety of PI3Kα-dependent diseases and disorders. In some embodiments, the disease of disorder is a cancer (e.g., breast cancer, brain cancer, prostate cancer, endometrial cancer, gastric cancer, leukemia, lymphoma, sarcoma, colorectal cancer, lung cancer, ovarian cancer, skin cancer, and head and neck cancer). In some embodiments, the disease or disorder associated with PI3Kα includes, but is not limited to, CLOVES syndrome (congenital lipomatous overgrowth, vascular malformations, epidermal naevi, scoliosis/skeletal and spinal syndrome), PIK3CA-related overgrowth syndrome (PROS), endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma.

### Combination Therapy

One or more additional therapeutic agents such as, for example, chemotherapeutics or other anti-cancer agents, anti-inflammatory agents, steroids, immunosuppressants, anesthetics (e.g., for use in combination with a surgical procedure), or other agents useful for treating diseases associated with PI3Kα can be used in combination with the compounds and salts provided herein. The agents can be combined with the present compounds in a single dosage form, or the agents can be administered simultaneously or sequentially as separate dosage forms.

Compounds described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, CDK4/6, MEKK, ERK, MAPK, mTOR, EGFR, HER2, HER3, HER4, INS—R, IGF-1R, IR—R, PDGFαR, PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the inhibitor as described herein can be combined with inhibitors of kinases associated with the PIK3/Akt/mTOR signaling pathway, such as PI3K, Akt (including Akt1, Akt2 and Akt3) and mTOR kinases.

For treating cancer and other proliferative diseases, compounds described herein can be used in combination with targeted therapies, including JAK kinase inhibitors (ruxolitinib, additional JAK1/2 and JAK1-selective, baricitinib or itacitinib), Pim kinase inhibitors (e.g., LGH447, INCB053914 and SGI-1776), PI3 kinase inhibitors including PI3K-delta selective and broad spectrum PI3K inhibitors (e.g., parsaclisib and INCB50797), PI3K-gamma inhibitors such as PI3K-gamma selective inhibitors, MEK inhibitors, CSFIR inhibitors (e.g., PLX3397 and LY3022855), TAM receptor tyrosine kinases inhibitors (Tyro-3, Axl, and Mer; e.g., INCB81776), angiogenesis inhibitors, interleukin receptor inhibitors, Cyclin Dependent kinase inhibitors (e.g., palbociclib, ribociclib, and abemaciclib), BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), estrogen receptor modulators (e.g., fulvestrant), androgen receptor modulators (e.g., enzalutamide), BCL2 inhibitors (e.g., venetoclax), hypoxia-inducible factor-2 alpha inhibitors (e.g., belzutifan), exportin-1 (XPO-1) inhibitors (e.g., selinexor), KRAS inhibitors (e.g., sotorasib), arginase inhibitors (e.g., INCB1158), indoleamine 2,3-dioxygenase inhibitors (e.g., epacadostat, NLG919 or BMS-986205), PARP inhibiors (e.g., olaparib or rucaparib), and inhibitors of BTK such as ibrutinib.

For treating cancer and other proliferative diseases, compounds described herein can be used in combination with chemotherapeutic agents, agonists or antagonists of nuclear receptors, or other anti-proliferative agents. Compounds described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of: abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

Example anti-inflammatory agents include, but are not limited to, aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

Example steroids include, but are not limited to, corticosteroids such as cortisone, dexamethasone, hydrocortisone, methylprednisolone, prednisolone, and prednisone.

Example immunosuppressants include, but are not limited to, azathioprine, chlorambucil, cyclophosphamide, cyclosporine, daclizumab, infliximab, methotrexate, and tacrolimus.

Example anesthetics include, but are not limited, to local anesthetics (e.g., lidocaine, procain, ropivacaine) and general anesthetics (e.g., desflurane, enflurane, halothane, isoflurane, methoxyflurane, nitrous oxide, sevoflurane, mmobarbital, methohexital, thiamylal, thiopental, diazepam, lorazepam, midazolam, etomidate, ketamine, propofol, alfentanil, fentanyl, remifentanil, buprenorphine, butorphanol, hydromorphone levorphanol, meperidine, methadone, morphine, nalbuphine, oxymorphone, pentazocine).

In some embodiments, the additional therapeutic agent is administered simultaneously with a compound or salt provided herein. In some embodiments, the additional therapeutic agent is administered after administration of the compound or salt provided herein. In some embodiments, the additional therapeutic agent is administered prior to administration of the compound or salt provided herein. In some embodiments, the compound or salt provided herein is administered during a surgical procedure. In some embodiments, the compound or salt provided herein is administered in combination with an additional therapeutic agent during a surgical procedure.

As provided herein, the additional compounds, inhibitors, agents, etc. can be combined with the compounds provided herein in a single or continuous dosage form, or they can be administered simultaneously or sequentially as separate dosage forms.

### Labeled Compounds and Assay Methods

Another aspect of the present invention relates to fluorescent dye, spin label, heavy metal or radio-labeled compounds of the invention that would be useful not only in imaging but also in assays, both in vitro and in vivo, for localizing and quantitating the PI3Kα enzyme in tissue samples, including human, and for identifying PI3Kα enzyme ligands by inhibition binding of a labeled compound. Accordingly, the present invention includes PI3Kα enzyme assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro FGFR enzyme labeling and competition assays, compounds that incorporate ³H, ¹⁴C, ⁸²Br, ¹²⁵I, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

One or more constituent atoms of the compounds presented herein can be replaced or substituted with isotopes of the atoms in natural or non-natural abundance. In some embodiments, one or more atoms are replaced or substituted by deuterium. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as -CD₃ being substituted for —CH₃). In some embodiments, alkyl groups of the disclosed Formulas (e.g., the compound of any of Formulas I-VIIb) can be perdeuterated.

In some embodiments, the compound provided herein (e.g., the compound of any of Formulas I-VIIb), or a pharmaceutically acceptable salt thereof, comprises at least one deuterium atom.

In some embodiments, the compound provided herein (e.g., the compound of any of Formulas I-VIIb), or a pharmaceutically acceptable salt thereof, comprises two or more deuterium atoms.

In some embodiments, the compound provided herein (e.g., the compound of any of Formulas I-VIIb), or a pharmaceutically acceptable salt thereof, comprises three or more deuterium atoms.

In some embodiments, for a compound provided herein (e.g., the compound of any of Formulas I-VIIb), or a pharmaceutically acceptable salt thereof, all of the hydrogen atoms are replaced by deuterium atoms (i.e., the compound is “perdeuterated”).

It is understood that a “radio-labeled” or “labeled compound” is a compound that has incorporated at least one radionuclide. In some embodiments the radionuclide is selected from the group consisting of ³H, ¹⁴C, ¹²⁵I, ³⁵S and ⁸²Br.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the PI3Kα enzyme. Accordingly, the ability of a test compound to compete with the radio-labeled compound for binding to the PI3Kα enzyme directly correlates to its binding affinity.

### Kits

The present invention also includes pharmaceutical kits useful, for example, in the treatment or prevention of PI3Kα-associated diseases or disorders referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples were found to be inhibitors of PI3Kα as described below.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

### Intermediate 1: 1-(3-Amino-6-chloropyridin-2-yl)piperidin-4-ol

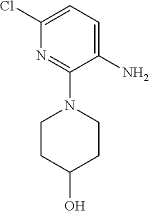 [SMILES: Nc1ccc(Cl)nc1N1CCC(O)CC1]

To a solution of 6-chloro-2-fluoro-3-nitro-pyridine (100.00 mg, 0.57 mmol) in 3 mL of EtOH was added piperidin-4-ol (57.30 mg, 0.57 mmol), stirred at 45° C. for 1 h. To this mixture was added potassium 2-methylpropan-2-olate (127.13 mg, 1.13 mmol) and 4,4,5,5-tetramethyl-2-(4,4,5,5-tetramethyl-1,3,2-dioxaborolan-2-yl)-1,3,2-dioxaborolane (431.54 mg, 1.70 mmol), After stirring at 80° C. for overnight, the mixture was diluted with water and extracted with EtOAc. The combined organics were washed with brine and dried with anhydrous sodium sulfate, filtered, and concentrated under reduced pressure. The residue was purified by silica gel column chromatography, eluted with DCM and ethyl acetate to provide the desired product as a yellow solid (90 mg, 70%). LCMS calculated for C₁₀H₁₅ClN₃O (M+H)⁺ m/z=228.1; found 228.1.

### Intermediates 2-5

Intermediates 2-5 in Table I-1 were prepared similarly as described for Intermediate 1.

TABLE I-1

| | | | LCMS |
|---|---|---|---|
| Intermediate | | | m/z |
| # | Chemical name | Structure | (M + H)⁺ |
| 2 | 1-(3-amino-6-chloropyridin-2-yl)-N- methylpiperidine-4-carboxamide | 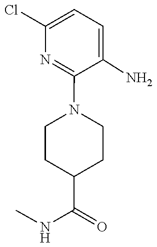 [SMILES: CNC(=O)C1CCN(c2nc(Cl)ccc2N)CC1] | 269.1 |
| 3 | 1-(4-(3-amino-6-chloropyridin-2- yl)piperazin-1-yl)ethan-1-one | 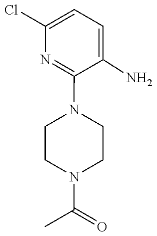 [SMILES: CC(=O)N1CCN(c2nc(Cl)ccc2N)CC1] | 255.1 |
| 4 | 6-chloro-2-morpholinopyridin-3-amine | 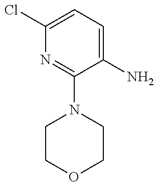 [SMILES: Nc1ccc(Cl)nc1N1CCOCC1] | 214.1 |
| 5 | 7-(3-amino-6-chloropyridin-2- yl)hexahydro-3H-oxazolo[3,4-a]pyrazin- 3-one | 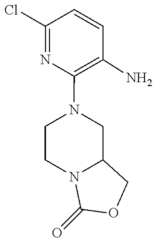 [SMILES: Nc1ccc(Cl)nc1N1CCN2C(=O)OCC2C1] | 269.1 |

### Intermediate 6: 6-Chloro-2-(1,3,4-oxadiazol-2-yl)pyridin-3-amine

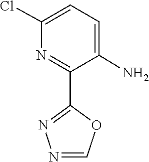 [SMILES: Nc1ccc(Cl)nc1-c1nnco1]

To a solution of 3-amino-6-chloropicolinic acid (100 mg, 0.58 mmol) in 2 mL of DCM was added (N-isocyanoimino)triphenylphosphorane (175 mg, 0.58 mmol). After stirring at r.t. for overnight, the mixture was diluted with water and extracted with DCM. The combined organics were washed with brine and dried with anhydrous sodium sulfate, filtered, and concentrated under reduced pressure. The residue was purified by silica gel column chromatography, eluted with DCM and ethyl acetate to provide the desired product as a yellow solid (84 mg, 74%). LCMS calculated for C₇H₆ClN₄O (M+H)⁺ m/z=197.0; found 197.0.

### Intermediate 7: 6-Chloro-2-(1-methyl-1H-1,2,4-triazol-3-yl)pyridin-3-amine

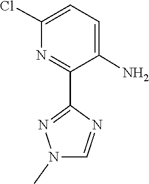

### Step 1: 3-amino-6-chloro-N′-methylpicolinimidohydrazide

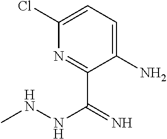

To a solution of 3-amino-6-chloro-pyridine-2-carbonitrile (500 mg, 3.26 mmol) in 10 mL of EtOH was added methylhydrazine (1.03 mL, 19.54 mml) at r.t. After stirring at 100° C. for 24 h, the solvent was removed under vacuum to provide the desired product as a yellow solid (600 mg, 92%). LCMS calculated for C₇H₁₁ClN₅ (M+H)⁺ m/z=200.1; found 200.1.

### Step 2: 6-chloro-2-(1-methyl-1H-1,2,4-triazol-3-yl)pyridin-3-amine

To a solution of 3-amino-6-chloro-N′-methylpicolinimidohydrazide (600 mg, 3 mmol) was dissolved in 5 mL of formic acid. After stirring at 105° C. or overnight, the solvent was removed under vacuum. Then to the residue was added 5 mL of ammonium hydroxide solution in water and EtOH, the mixture was stirred for overnight at 100° C. The mixture was diluted with water and extracted with EtOAc. The combined organics were washed with brine and dried with anhydrous sodium sulfate, filtered, and concentrated under reduced pressure. The residue was precipitated in ether, filtered and washed with ether to provide the desired product as a yellow solid (420 mg, 67%). LCMS calculated for C₈H₉ClN₅ (M+H)⁺ m/z=210.1; found 210.1.

### Intermediate 8: 2-(3-(3-Amino-6-chloropyridin-2-yl)-1H-1,2,4-triazol-1-yl) ethan-1-ol

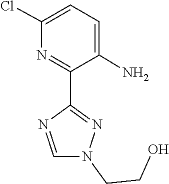 [SMILES: Nc1ccc(Cl)nc1-c1ncn(CCO)n1]

The titled compound was prepared using similar procedures as described for Intermediate 7 with 2-hydrazineylethan-1-ol replacing methylhydrazine in Step 1. LCMS calculated for C₉H₁₁ClN₅O (M+H)⁺ m/z=240.1; found 240.1.

### Intermediate 9: 6-Chloro-2-(4-methyl-4H-1,2,4-triazol-3-yl)pyridin-3-amine

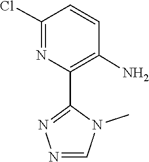

4-methyl-1,2,4-triazole (120.16 mg, 1.45 mmol) was dissolved in 2 mL of THF and cooled to −78° C. n-Butyllithium (578.44 uL, 1.45 mmol) was added via a syringe. After stirring for 10 min, dichlorozinc (2M THF solution, 761.10 uL, 1.45 mmol) was added. The reaction mixture was stirred at −78° C. for 20 min, then warmed to room temperature. The resulting mixture was transferred via a syringe into a pressure flask which contained a mixture of 2-bromo-6-chloro-pyridin-3-amine (100.0 mg, 0.48 mmol), tetrakis(triphenylphosphine)palladium (55.70 mg, 0.05 mmol) and 3 mL of dioxane. The reaction mixture was heated at 120° C. in the sealed flask for 15 h, and then cooled to r.t. EtOAc was added to quench the reaction. The resulting mixture was washed with water. The organic layer was concentrated and purified with silica gel column to give product as yellow solid (80 mg, 79%). LCMS calculated for C₈H₉ClN₅ (M+H)⁺ m/z=210.1; found 210.1.

### Intermediate 10: 6-Chloro-2-(1H-tetrazol-5-yl)pyridin-3-amine

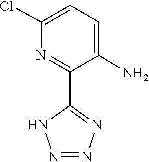 [SMILES: Nc1ccc(Cl)nc1-c1nnn[nH]1]

To a solution of 3-amino-6-chloro-pyridine-2-carbonitrile (1.54 g, 10.00 mmol) in 20 mL DMF was added sodium azide (1.3 g, 20.00 mmol) and ammonia hydrochloride (1.7 g, 20.00 mmol) in one portion. After stirring at 120° C. for 12 h, the solvent was removed under vacuum. The residue was dissolved in water and adjusted the pH to 2 by adding 10% HCl. The precipitate was collected by filtration to give product as white solid (1.8 g, 92%). LCMS calculated for C₆H₆ClN₆ (M+H)⁺ m/z=197.0; found 197.0.

### Intermediate 11: 6-Chloro-2-(2-methyl-2H-tetrazol-5-yl)pyridin-3-amine

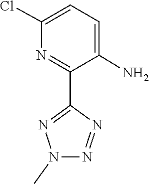

### Step 1: 2-(6-chloro-2-(1H-tetrazol-5-yl)pyridin-3-yl) isoindoline-1,3-dione

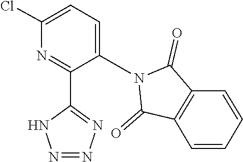 [SMILES: O=C1c2ccccc2C(=O)N1c1ccc(Cl)nc1-c1nnn[nH]1]

To a solution of 6-chloro-2-(1H-tetrazol-5-yl)pyridin-3-amine (Intermediate 10) (1.8 g, 9.16 mmol) in 10 mL of acetic acid was added isobenzofuran-1,3-dione (3.4 g, 22.89 mmol) at r.t. After stirring at 120° C. for overnight, the solvent was removed under vacuum to provide the desired product as a yellow solid (2.9 g, 97%). LCMS calculated for C₁₄H₈ClN₆O₂ (M+H)⁺ m/z=327.0; found 327.0.

### Step 2: 2-(6-chloro-2-(2-methyl-2H-tetrazol-5-yl)pyridin-3-yl) isoindoline-1,3-dione

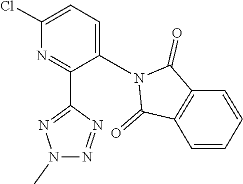 [SMILES: Cn1nnc(-c2nc(Cl)ccc2N2C(=O)c3ccccc3C2=O)n1]

To a solution of 2-[6-chloro-2-(1H-tetrazol-5-yl)-3-pyridyl]isoindoline-1,3-dione (3.00 g, 9.18 mmol) in 10 mL DMF was added iodomethane (0.86 mL, 1.96 g, 13.77 mmol) and dipotassium carbonate (3.81 g, 27.55 mmol). After stirring at r.t. for 2 h, the mixture was diluted with water and extracted with EtOAc. The combined organics were washed with brine and dried with anhydrous sodium sulfate, filtered, and concentrated under reduced pressure. The resulting mixture was purified by silica gel column chromatography, eluted with DCM and ethyl acetate to provide the desired product as yellow solid (540 mg, 17%) and another regioisomer as yellow solid (570 mg, 18%). LCMS calculated for C₁₅H₁₀ClN₆O₂ (M+H)⁺ m/z=341.1; found 341.1.

### Step 3: 6-chloro-2-(2-methyl-2H-tetrazol-5-yl)pyridin-3-amine

To a solution of 2-(6-chloro-2-(2-methyl-2H-tetrazol-5-yl)pyridin-3-yl) isoindoline-1,3-dione (540 mg, 1.58 mmol) was added 10 mL of hydrazine monohydrate. After stirring at 45° C. for 2 h, the resulting mixture was diluted with water. The precipitate was filtered and washed with water to give the product as white solid (330 mg, 100%). LCMS calculated for C₇H₈CN₆ (M+H)⁺ m/z=211.0; found 211.0.

### Intermediate 12: 1-(1-methylazetidin-3-yl)-4-(4,4,5,5-tetramethyl-1,3,2-dioxaborolan-2-yl)-1H-pyrazole

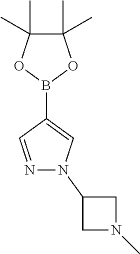

### Step 1: 1-(azetidin-3-yl)-4-(4,4,5,5-tetramethyl-1,3,2-dioxaborolan-2-yl)-1H-pyrazole

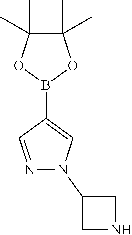 [SMILES: CC1(C)OB(c2cnn(C3CNC3)c2)OC1(C)C]

Dissolved tert-butyl 3-(4-(4,4,5,5-tetramethyl-1,3,2-dioxaborolan-2-yl)-1H-pyrazol-1-yl) azetidine-1-carboxylate (500 mg, 1.43 mmol) in 30% TFA in DCM. After stirring at r.t. for 1 h, the solvent was removed under vacuum to give product (360 mg, 100%). LCMS calculated for C₁₂H₂₁BN₃O₂ (M+H)⁺ m/z=250.2; found 250.2.

### Step 2: 1-(1-methylazetidin-3-yl)-4-(4,4,5,5-tetramethyl-1,3,2-dioxaborolan-2-yl)-1H-pyrazole

To a solution of 1-(azetidin-3-yl)-4-(4,4,5,5-tetramethyl-1,3,2-dioxaborolan-2-yl) pyrazole (50.00 mg, 0.20 mmol) in 1 mL of ACN and 0.5 mL AcOH was added formaldehyde (73.94 uL, 60.26 mg, 2.01 mmol) and sodium triacetoxyborohydride (212.69 mg, 1.00 mmol). After stirring at r.t. for 30 min, the mixture was quenched with water and extracted with EtOAc. The combined organics were washed with brine and dried with anhydrous sodium sulfate, filtered, and concentrated under reduced pressure. The resulting mixture was purified by silica gel column chromatography, eluted with DCM and ethyl acetate to provide the desired product as white solid (43 mg, 81%). LCMS calculated for C₁₃H₂₃BN₃O₂(M+H)⁺ m/z=264.2; found 264.2.

### Intermediates 13-14

Intermediates 13-14 in Table I-2 were prepared similarly as described for Intermediate 12.

TABLE I-2

| Intermediate | | | LCMS |
|---|---|---|---|
| # | Chemical name | Structure | m/z (M + H)⁺ |
| 13 | 1-(1-methylpyrrolidin-3-yl)-4-(4,4,5,5- tetramethyl-1,3,2-dioxaborolan-2-yl)-1H- pyrazole | 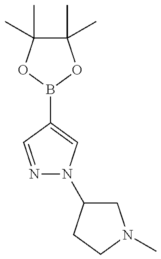 | 278.2 |
| 14 | 1-methyl-3-(4-(4,4,5,5-tetramethyl-1,3,2- dioxaborolan-2-yl)-1H-pyrazol-1- yl)piperidine | 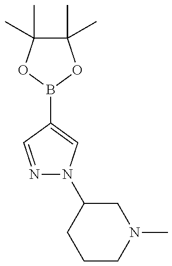 | 292.2 |

### Intermediate 15: N,N-Dimethyl-4-(4-(4,4,5,5-tetramethyl-1,3,2-dioxaborolan-2-yl)-1H-pyrazol-1-yl)piperidine-1-carboxamide

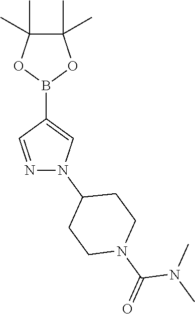

To a solution of 4-(4-(4,4,5,5-tetramethyl-1,3,2-dioxaborolan-2-yl)-1H-pyrazol-1-yl)piperidine (100 mg, 0.36 mmol) in 3 mL ACN was added dimethylcarbamic chloride (77 mg, 0.72 mmol) and triethylamine (0.15 mL, 1.1 mmol). After stirring at r.t. for 30 min, the mixture was quenched with water and extracted with EtOAc. The combined organics were washed with brine and dried with anhydrous sodium sulfate, filtered, and concentrated under reduced pressure. The resulting mixture was purified by silica gel column chromatography, eluted with DCM and ethyl acetate to provide the desired product as white solid (102 mg, 81%). LCMS calculated for C₁₇H₃₀BN₄O₃(M+H)⁺ m/z=349.2; found 349.2.

### Intermediate 16: 3-(4-(4-(4,4,5,5-Tetramethyl-1,3,2-dioxaborolan-2-yl)-1H-pyrazol-1-yl)piperidin-1-yl)propanenitrile

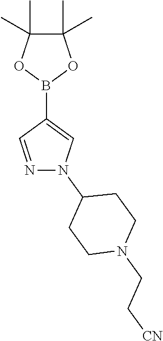 [SMILES: CC1(C)OB(c2cnn(C3CCN(CCC#N)CC3)c2)OC1(C)C]

To a solution of 4-(4-(4,4,5,5-tetramethyl-1,3,2-dioxaborolan-2-yl)-1H-pyrazol-1-yl)piperidine (50 mg, 0.18 mmol) in 1 mL ACN was added acrylonitrile (47 mg, 0.2 mmol). After stirring at r.t. for 30 min, the mixture was quenched with water and extracted with EtOAc. The combined organics were washed with brine and dried with anhydrous sodium sulfate, filtered, and concentrated under reduced pressure. The resulting mixture was purified by silica gel column chromatography, eluted with DCM and ethyl acetate to provide the desired product as white solid (26 mg, 44%). LCMS calculated for C₁₇H₂₈BN₄O₂(M+H)⁺ m/z=331.2; found 331.2.

### Intermediate 17: tert-Butyl 3-(3-amino-6-chloropyridin-2-yl)-6,7-dihydropyrazolo[1,5-a]pyrazine-5(4H)-carboxylate

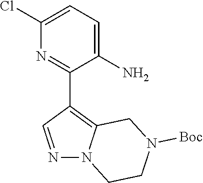 [SMILES: CC(C)(C)OC(=O)N1CCn2ncc(-c3nc(Cl)ccc3N)c2C1]

### Step 1: tert-butyl 3-(4,4,5,5-tetramethyl-1,3,2-dioxaborolan-2-yl)-6,7-dihydropyrazolo[1,5-a]pyrazine-5(4H)-carboxylate

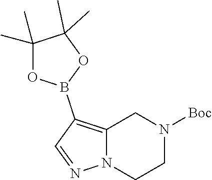 [SMILES: CC(C)(C)OC(=O)N1CCn2ncc(B3OC(C)(C)C(C)(C)O3)c2C1]

Under nitrogen, to a mixture of tert-butyl 3-bromo-6,7-dihydropyrazolo[1,5-a]pyrazine-5(4H)-carboxylate (100 mg, 0.33 mmol) and bis(pinacolato)diboron (100.85 mg, 0.40 mmol) was added potassium acetate (65 mg, 0.66 mmol), Pd(dppf)Cl2·DCM (54.0 mg, 0.07 mmol) and anhydrous 1,4-dioxane (3.3 mL) at room temperature. The resulting mixture was heated at 100° C. for 3 hours. Upon completion of reaction, the mixture was cooled to room temperature, solvent was removed under reduced pressure. The residue was purified with silica gel chromatography, eluting with 0-100% ethyl acetate/hexanes to give desired product as white solid (75.7 mg, 66%), LCMS calculated for C₁₇H₂₉BN₃O₄(M+H)⁺ m/z=350.2; found: 350.1;

### Step 2: tert-Butyl 3-(3-amino-6-chloropyridin-2-yl)-6,7-dihydropyrazolo[1,5-a]pyrazine-5(4H)-carboxylate

At room temperature, to a mixture of tert-butyl 3-(4,4,5,5-tetramethyl-1,3,2-dioxaborolan-2-yl)-6,7-dihydropyrazolo[1,5-a]pyrazine-5(4H)-carboxylate (75.8 mg, 0.22 mmol) and 2-bromo-6-chloropyridin-3-amine (50.0 mg, 0.24 mmol) was added tetrakis(triphenylphosphine)palladium(0) (55.7 mg, 0.05 mmol), cesium carbonate (157.1 mg, 0.48 mmol), and 5 to 1 mixture of 1,4-dioxane and water (2.4 mL). The resulting mixture was heated at 80° C. for 1 hour. Upon completion of reaction, the mixture was cooled to room temperature. Solvent was removed under reduced pressure. The residue was purified with silica gel chromatography, eluting with 0-100% Ethyl acetate/Hexanes to give desired product as yellow solid (40.3 mg, 48%). LCMS calculated for C₁₆H₂₁ClN₅O₂ (M+H)⁺ m/z=350.1; found: 350.1;

### Intermediate 18: 6-Chloro-2-(1-methyl-1H-pyrazol-4-yl)pyridin-3-amine

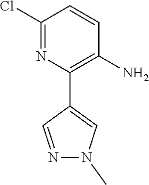

To a solution of 2-bromo-6-chloropyridin-3-amine (100 mg, 0.48 mmol) in 2.5 mL of dioxane and 0.5 mL of water was added (1-methyl-1H-pyrazol-4-yl) boronic acid (73 mg, 0.58 mmol), potassium carbonate (133 mg, 0.96 mmol) and tetrakis(triphenylphosphine)palladium (111 mg, 0.1 mmol) under nitrogen. After stirring at 85° C. for 1 h, the mixture was diluted with water and extracted with EtOAc. The combined organics were washed with brine and dried with anhydrous sodium sulfate, filtered, and concentrated under reduced pressure. The residue was purified by silica gel column chromatography, eluted with DCM and ethyl acetate to provide the desired product as a yellow solid (82 mg, 81%). LCMS calculated for C₉H₁₀ClN₄ (M+H)⁺ m/z=209.1; found 209.1.

### Intermediates 19-50

Intermediates 19-34 in Table I-3 were prepared similarly as described for Intermediate 18.

TABLE I-3

| Intermediate | | | LCMS |
|---|---|---|---|
| # | Chemical name | Structure | m/z (M + H)⁺ |
| 19 | tert-butyl 4-(3-amino-6-chloropyridin-2- yl)-1H-pyrazole-1-carboxylate | 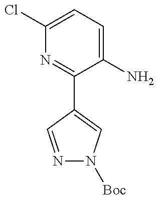 [SMILES: CC(C)(C)OC(=O)n1cc(-c2nc(Cl)ccc2N)cn1] | 295.1 |
| 20 | 6-chloro-2-(1-ethyl-1H-pyrazol-4- yl)pyridin-3-amine | 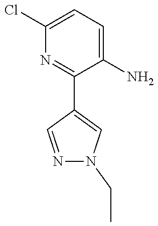 | 223.1 |
| 21 | 6-chloro-2-(1,3-dimethyl-1H-pyrazol-4- yl)pyridin-3-amine | 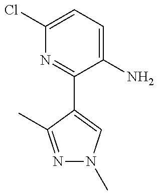 | 223.1 |
| 22 | tert-butyl 4-(3-amino-6-chloropyridin-2- yl)-3-methyl-1H-pyrazole-1-carboxylate | 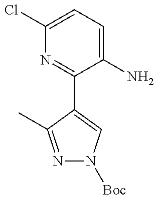 [SMILES: Cc1nn(C(=O)OC(C)(C)C)cc1-c1nc(Cl)ccc1N] | 309.1 |
| 23 | 6-chloro-2-(1,5-dimethyl-1H-pyrazol-4- yl)pyridin-3-amine | 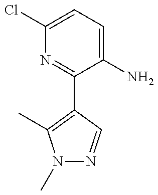 | 223.1 |
| 24 | 3-amino-6-chloro-1′-methyl-[2,3′- bipyridin]-6′(1′H)-one | 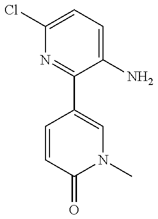 [SMILES: Cn1cc(-c2nc(Cl)ccc2N)ccc1=O] | 236.1 |
| 25 | 5-(2-amino-5-chlorophenyl)-N- methylpicolinamide | 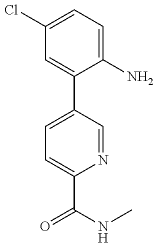 [SMILES: CNC(=O)c1ccc(-c2cc(Cl)ccc2N)cn1] | 262.1 |
| 26 | 6-chloro-3′-fluoro-[1,4′-bipyridin]-3-amine | 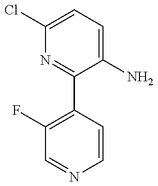 [SMILES: Nc1ccc(Cl)nc1-c1ccncc1F] | 224.1 |
| 27 | 6-chloro-[2,3′-bipyridin]-3-amine | 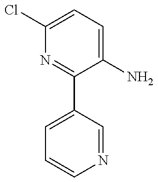 [SMILES: Nc1ccc(Cl)nc1-c1cccnc1] | 206.1 |
| 28 | 3-amino-6-chloro-1′-methyl-[2,4′- bipyridin]-2′-(1′H)-one | 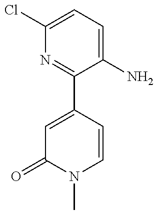 [SMILES: Cn1ccc(-c2nc(Cl)ccc2N)cc1=O] | 236.1 |
| 29 | 6-chloro-2′-methyl-[2,4′-bipyridin]-3- amine | 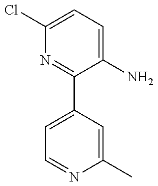 | 220.1 |
| 30 | 4-(3-amino-6-chloropyridin-2-yl)-2-fluoro- N-methylbenzamide | 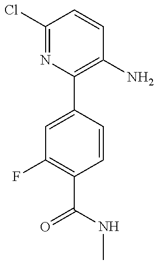 [SMILES: CNC(=O)c1ccc(-c2nc(Cl)ccc2N)cc1F] | 280.1 |
| 31 | 6-chloro-2-(pyrimidin-5-yl)pyridin-3- amine | 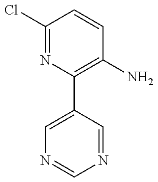 [SMILES: Nc1ccc(Cl)nc1-c1cncnc1] | 207.1 |
| 32 | 5-(3-amino-6-chloropyridin-2-yl)-2- methylisoindolin-1-one | 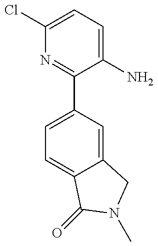 [SMILES: CN1Cc2cc(-c3nc(Cl)ccc3N)ccc2C1=O] | 274.1 |
| 33 | 6-chloro-2-(1-methyl-1H-indazol-5- yl)pyridin-3-amine | 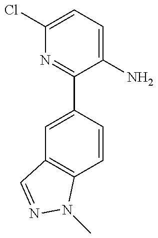 [SMILES: Cn1ncc2cc(-c3nc(Cl)ccc3N)ccc21] | 259.1 |
| 34 | 6-chloro-2-(quinolin-6-yl)pyridin-3-amine | 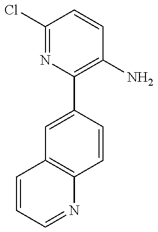 [SMILES: Nc1ccc(Cl)nc1-c1ccc2ncccc2c1] | 256.1 |
| 35 | 6-chloro-2-(1-(tetrahydro-2H-pyran-4-yl)- 1H-pyrazol-4-yl)pyridin-3-amine | 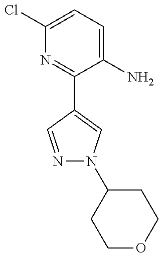 [SMILES: Nc1ccc(Cl)nc1-c1cnn(C2CCOCC2)c1] | 279.1 |
| 36 | 6-chloro-2′-methoxy-[2,4′-bipyridin]-3- amine | 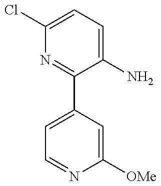 [SMILES: COc1cc(-c2nc(Cl)ccc2N)ccn1] | 236.1 |
| 37 | 6-chloro-6′-methoxy-[2,3′-bipyridin]-3- amine | 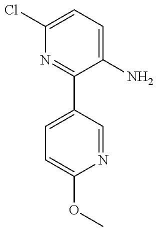 [SMILES: COc1ccc(-c2nc(Cl)ccc2N)cn1] | 236.1 |
| 38 | 6-chloro-N2′,N2′-dimethyl-[2,4′- bipyridine]-2′,3-diamine | 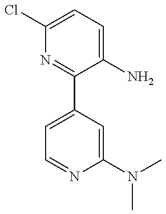 [SMILES: CN(C)c1cc(-c2nc(Cl)ccc2N)ccn1] | 249.1 |
| 39 | 4-(3-amino-6-chloropyridin-2-yl)-3- fluorobenzonitrile | 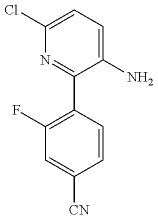 [SMILES: N#Cc1ccc(-c2nc(Cl)ccc2N)c(F)c1] | 248.1 |
| 40 | 4-(3-amino-6-chloropyridin-2- yl)benzonitrile | 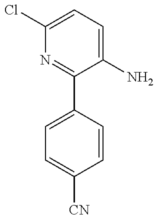 [SMILES: N#Cc1ccc(-c2nc(Cl)ccc2N)cc1] | 230.1 |
| 41 | 4-(3-amino-6-chloropyridin-2-yl)benzoic acid | 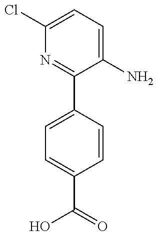 [SMILES: Nc1ccc(Cl)nc1-c1ccc(C(=O)O)cc1] | 249.1 |
| 42 | 6-(3-amino-6-chloropyridin-2-yl)-2- methyl-3,4-dihydroisoquinolin-1(2H)-one | 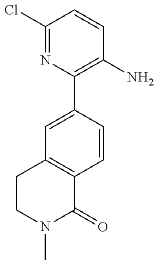 [SMILES: CN1CCc2cc(-c3nc(Cl)ccc3N)ccc2C1=O] | 288.1 |
| 43 | 4-(3-amino-6-methylpyridin-2-yl)-2- fluoro-N-methylbenzamide | 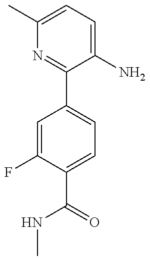 [SMILES: CNC(=O)c1ccc(-c2nc(C)ccc2N)cc1F] | 260.1 |
| 44 | 2-(1-cyclopropyl-1H-pyrazol-4-yl)-6- methylpyridin-3-amine | 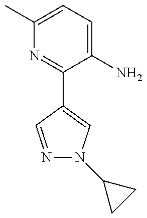 [SMILES: Cc1ccc(N)c(-c2cnn(C3CC3)c2)n1] | 215.1 |
| 45 | 6-methyl-2-(1-(tetrahydro-2H-pyran-4-yl)- 1H-pyrazol-4-yl)pyridin-3-amine | 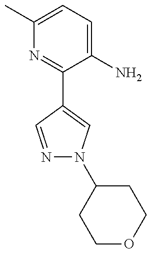 [SMILES: Cc1ccc(N)c(-c2cnn(C3CCOCC3)c2)n1] | 259.2 |
| 46 | 2′,6-dimethyl-[2,4′-bipyridin]-3-amine | 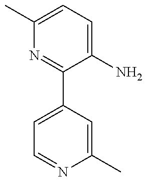 | 200.1 |
| 47 | 2-(4-(3-amino-6-methylpyridin-2-yl)-1H- pyrazol-1-yl)ethan-1-ol | 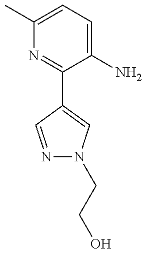 [SMILES: Cc1ccc(N)c(-c2cnn(CCO)c2)n1] | 219.1 |
| 48 | tert-butyl 4-(3-amino-6-methylpyridin-2- yl)-3-methyl-1H-pyrazole-1-carboxylate | 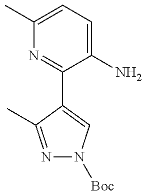 [SMILES: Cc1ccc(N)c(-c2cn(C(=O)OC(C)(C)C)nc2C)n1] | 289.2 |
| 49 | 4-fluoro-2-(1-methyl-1H-pyrazol-4- yl)aniline | 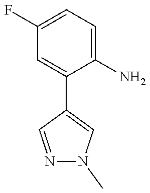 | 192.1 |
| 50 | 6-methyl-2-(1-methyl-1H-pyrazol-4- yl)pyridin-3-amine | 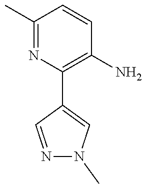 | 189.1 |

### Intermediate 51: 1-(2-((Triisopropylsilyl)oxy)ethyl)-1H-pyrazol-5-amine

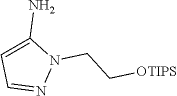

To a stirred solution of 2-(5-aminopyrazol-1-yl) ethanol (7.50 g, 58.99 mmol) in 100 mL DCM was added chloro(triisopropyl)silane (18.93 mL, 88.48 mmol), imidazole (8.03 g, 117.98 mmol) and N,N-dimethylpyridin-4-amine (1.08 g, 8.85 mmol) at 0° C. The reaction was stirred at rt for 12 h. LCMS analysis indicated the reaction was complete. The reaction was quenched with 100 mL water and extracted with 2×100 mL DCM. The combined organics were washed with saturated NaCl, dried with anhydrous sodium sulfate, filtered, and concentrated under reduced pressure. The residue was purified by silica gel column chromatography, eluted with 0˜10% EtOAc in DCM to provide the desired product (13.00 g, 77.7%). LCMS calculated for C₁₄H₃₀N₃OSi (M+H)⁺ m/z=284.2; found 284.2.

### Intermediate 52: 9-Bromo-4-cyclopropyl-3-ethyl-7-methyl-3,4-dihydro-5H-pyrazolo[3,4-c]isoquinolin-5-one

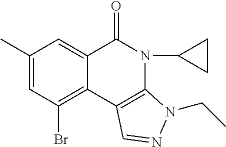 [SMILES: CCn1ncc2c3c(Br)cc(C)cc3c(=O)n(C3CC3)c21]

### Step 1: 3-bromo-N-cyclopropyl-2-iodo-5-methyl-benzamide

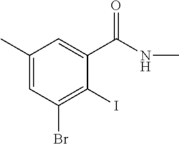

To a solution of 3-bromo-2-iodo-5-methyl-benzoic acid (2.0 g, 5.87 mmol) in 30 mL DMF was added cyclopropylamine (401.91 mg, 7.04 mmol) followed by HATU (3.3 g, 8.80 mmol) and N-ethyl-N-isopropyl-propan-2-amine (2.04 mL, 11.73 mmol). The resulting solution was stirred at r.t. for 1 h, then poured into a mixture of 200 mL EtOAc and 200 mL water. The aqueous phase was washed with 50 mL ethyl acetate 3 times. The combined organics were washed with sat. NaCl and dried with anhydrous sodium sulfate, filtered, and concentrated under reduced pressure. The residue was purified by silica gel column chromatography, eluted with EtOAc/DCM (0 to 100%) to provide the desired product. LCMS calculated for C₁₁H₁₂BrINO (M+H)⁺ m/z=379.9; found 379.9.

### Step 2: 9-bromo-4-cyclopropyl-3-ethyl-7-methyl-pyrazolo[3,4-c]isoquinolin-5-one

To a 250 mL round bottom flask was added 3-bromo-N-cyclopropyl-2-iodo-5-methyl-benzamide (1.80 g, 4.74 mmol) and 1-ethyl-5-iodo-pyrazole (1.05 g, 4.74 mmol), followed by tris(2-furyl)phosphane (219.94 mg, 0.95 mmol), Pd(OAc)₂ (106.34 mg, 0.47 mmol) and K₂CO₃ (1309.25 mg, 9.47 mmol). The flask was sealed with a rubber septum, evacuated and backfilled with nitrogen for three times. To this flask was added 90 mL DMF. The resulting solution was heated to 105° C. for 16 h under nitrogen atmosphere. Upon cooling to room temperature, the mixture was diluted with water and extracted with EtOAc three times. The combined organics were washed with sat. NaCl and dried with anhydrous sodium sulfate, filtered, and concentrated under reduced pressure. The residue was purified by silica gel column chromatography, eluted with EtOAc/DCM (0 to 100%) to provide the desired product. LCMS calculated for C₁₆H₁₇BrN₃O (M+H)⁺ m/z=346.1; found page 345.9.

### Intermediate 53: 9-Bromo-3-ethyl-7-methyl-4-(tetrahydrofuran-3-yl)-3,4-dihydro-5H-pyrazolo[3,4-c]isoquinolin-5-one

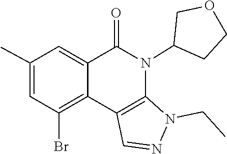 [SMILES: CCn1ncc2c3c(Br)cc(C)cc3c(=O)n(C3CCOC3)c21]

The title compound was prepared using similar procedures as described for Intermediate 52 with tetrahydrofuran-3-amine replacing cyclopropylamine in Step 1. The crude product was purified by silica gel column chromatography, eluted with EtOAc/DCM (0 to 100%) to provide the desired product. LCMS calculated for C₁₇H₁₉BrN₃O₂ (M+H)⁺ m/z=376.1; found page 376.1.

### Intermediate 54: Benzyl 3-hydrazineylpiperidine-1-carboxylate

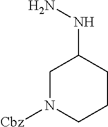 [SMILES: NNC1CCCN(C(=O)OCc2ccccc2)C1]

### Step 1: benzyl 3-(2-(tert-butoxycarbonyl) hydrazineyl)piperidine-1-carboxylate

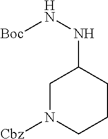 [SMILES: CC(C)(C)OC(=O)NNC1CCCN(C(=O)OCc2ccccc2)C1]

To a mixture of benzyl 3-oxopiperidine-1-carboxylate (5.0 g, 21.4 mmol) was added MeOH (300 mL), tert-butyl hydrazinecarboxylate (2.9 g, 22 moml), and acetic acid (3 mL), and sodium cyanoborohydride (1.4 g, 22.8 mmol). The mixture was stirred for 12 h at rt, then quenched sat. NaHCO₃. The mixture was extracted with DCM/IPA 3:1 for three times. The combined organics were washed with sat. NaCl and dried with anhydrous sodium sulfate, filtered, and concentrated under reduced pressure. The residue was purified by silica gel column chromatography, eluted with DCM/MeOH to provide the desired product. LCMS calculated for C₁₈H₂₈N₃O₄ (M+H)⁺ m/z=350.2; found page 350.2.

### Step 2: benzyl 3-hydrazinopiperidine-1-carboxylate

To a stirred solution of benzyl 3-(2-tert-butoxycarbonylhydrazino)piperidine-1-carboxylate (15.00 g, 42.93 mmol) in 50 mL DCM was added 50 mL TFA at rt. The resulting mixture was kept stirred at rt overnight. LCMS analysis indicated the reaction was complete. The reaction mixture was concentrated under reduced pressure. The residue was used directly in the following step without further purification. LCMS calculated for C₁₃H₂₀N₃O₂ (M+H)⁺ m/z=250.2; found page 250.2.

### Intermediates 55-56

Intermediates 39-40 in Table I-4 were prepared similarly as described for intermediate 38.

TABLE I-4

| Intermediate | | | LCMS |
|---|---|---|---|
| # | Chemical name | Structure | m/z (M + H)⁺ |
| 55 | benzyl 4- (hydrazineylmethyl)piperidine-1- carboxylate | 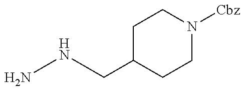 [SMILES: NNCC1CCN(C(=O)OCc2ccccc2)CC1] | 264.2 |
| 56 | benzyl 3- (hydrazineylmethyl)pyrrolidine-1- carboxylate | 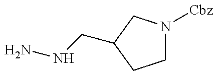 [SMILES: NNCC1CCN(C(=O)OCc2ccccc2)C1] | 250.2 |

### Preparation of Provided Compounds

### Example 1: 2-((1-(4,7-dimethyl-5-oxo-4,5-dihydropyrazolo[1,5-a]quinazolin-9-yl)ethyl)amino)benzoic acid (I-1)

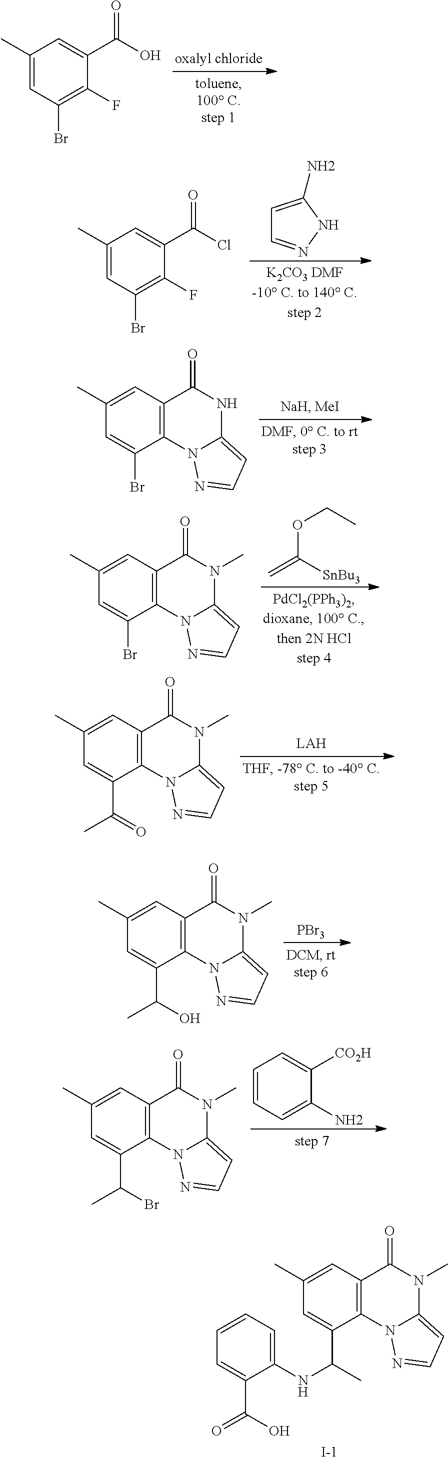

### Step 1: 3-bromo-2-fluoro-5-methylbenzoyl chloride

To a mixture of 3-bromo-2-fluoro-5-methylbenzoic acid (1.0 g, 4.29 mmol) in toluene (100 mL) were added oxalyl dichloride (1.09 g, 8.58 mmol) and 0.1 mL DMF. The resulting mixture was stirred at 100° C. for 3 h. Upon cooling to room temperature, the reaction was concentrated under reduced pressure. The residue was used directly in next step without further purification.

### Step 2: 9-bromo-7-methylpyrazolo[1,5-a]quinazolin-5(4H)-one

The mixture of 3-bromo-2-fluoro-5-methylbenzoyl chloride (0.9 g, 3.6 mmol) in DMF (50 mL) was added 1H-pyrazol-5-amine (427 mg, 5.15 mmol) and K₂CO₃ (1.18 g, 8.58 mmol) at −10° C. The reaction was then slowly warmed to rt and stirred for 30 min. After completion, the reaction was heated to 140° C. and stirred at this temperature for 6 h before quenched with water and extracted with EtOAc. The combined organics were removed partially, and the resulting solid was collected to afford pure product as a light brown solid (320 mg, 32%). LCMS calculated for C₁₁H₉BrN₃O (M+H)⁺ m/z=278.0; found 278.1.

### Step 3: 9-bromo-4,7-dimethylpyrazolo[1,5-a]quinazolin-5(4H)-one

To a mixture of 9-bromo-7-methylpyrazolo[1,5-a]quinazolin-5(4H)-one (320 mg, 1.15 mmol) in DMF (20 mL) was added NaH (50 mg, 1.2 mmol) at 0° C. under nitrogen atmosphere. The resulting mixture was stirred at same temperature for 30 min MeI (213 mg, 1.5 mmol) was added. The reaction mixture was then slowly warmed to rt and stirred for 4 h. After completion, the reaction was carefully quenched with water at 0° C. while vigorously stirred. The mixture was then extracted with ethyl acetate (2×300 mL). The combined organics were dried with anhydrous sodium sulfate, filtered, and concentrated under reduced pressure. The residue was purified by silica gel column chromatography, eluted with hexanes and ethyl acetate to provide the desired product, which was further purified by silica gel column chromatography, eluted with DCM and ethyl acetate to provide the desired product as a brown oil (210 mg, 65%). LCMS calculated for C₁₂H₁BrN₃O (M+H)⁺ m/z=292.0; found 292.0.

### Step 4: 9-acetyl-4,7-dimethylpyrazolo[1,5-a]quinazolin-5(4H)-one

The mixture of 9-bromo-4,7-dimethylpyrazolo[1,5-a]quinazolin-5(4H)-one (100 mg, 0.34 mmol), PdCl₂(PPh₃)₂ (23 mg, 0.03 mmol), and tributyl(1-ethoxyvinyl)stannane (143 mg, 0.4 mmol) in dioxane (2 mL) was heated at 100° C. for 12 h under nitrogen atmosphere. Upon cooling to room temperature, the mixture was added 2 N HCl and stirred for 30 min. Then 200 mg CsF was added and stirred for another 30 min before the mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a brown oil (60 mg, 69%). LCMS calculated for C₁₄H₁₄N₃O₂ (M+H)⁺ m/z=256.1; found 256.1.

### Step 5: 9-(1-hydroxyethyl)-4,7-dimethylpyrazolo[1,5-a]quinazolin-5(4H)-one

To a mixture of 9-acetyl-4,7-dimethylpyrazolo[1,5-a]quinazolin-5(4H)-one (60 mg, 0.23 mmol) in THF (2 mL) was added LAH (0.3 mL, 0.3 mmol, 1 M solution in THF) at −78° C. The resulting mixture was stirred at same temperature for 10 min, and then warmed to −40° C. before quenched with sat. NH₄Cl. The mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a colorless oil (20 mg, 33%). LCMS calculated for C₁₄H₁₄N₃O (M−OH)⁺ m/z=240.1; found 240.1.

### Step 6: 9-(1-bromoethyl)-4,7-dimethylpyrazolo[1,5-a]quinazolin-5(4H)-one

To a mixture of 9-(1-hydroxyethyl)-4,7-dimethylpyrazolo[1,5-a]quinazolin-5(4H)-one (20 mg, 0.08 mmol) in DCM (1 mL) was added PBr₃ (27 mg, 0.1 mmol). The resulting mixture was stirred at rt for 2 h. The mixture was then quenched with sat. NaHCO₃ and extracted with DCM. The combined organics were washed with sat. NaCl, dried with anhydrous sodium sulfate, filtered, and concentrated under reduced pressure. The residue was used directly in next step without further purification.

### Step 7: 2-((1-(4,7-dimethyl-5-oxo-4,5-dihydropyrazolo[1,5-a]quinazolin-9-yl)ethyl)amino)benzoic acid (I-1)

9-(1-bromoethyl)-4,7-dimethylpyrazolo[1,5-a]quinazolin-5(4H)-one (20 mg, 0.06 mmol) in 2 dram vial was added DMF (0.3 mL), and 2-aminobenzoic acid (20 mg, 0.14 mmol). The mixture was heated to 90° C. for 2 h. Upon cooling to room temperature, the mixture was diluted with MeOH and purified by prep-HPLC (column: Sunfire prep C18 column, 30*150 mm, 5 um; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₁H₂₁N₄O₃ (M+H)⁺ m/z=377.1; found 377.2.

### Example 2: 2-((1-(4,7-dimethyl-5-oxo-3-phenyl-4,5-dihydropyrazolo[1,5-a]quinazolin-9-yl)ethyl)amino)benzoic acid (I-2)

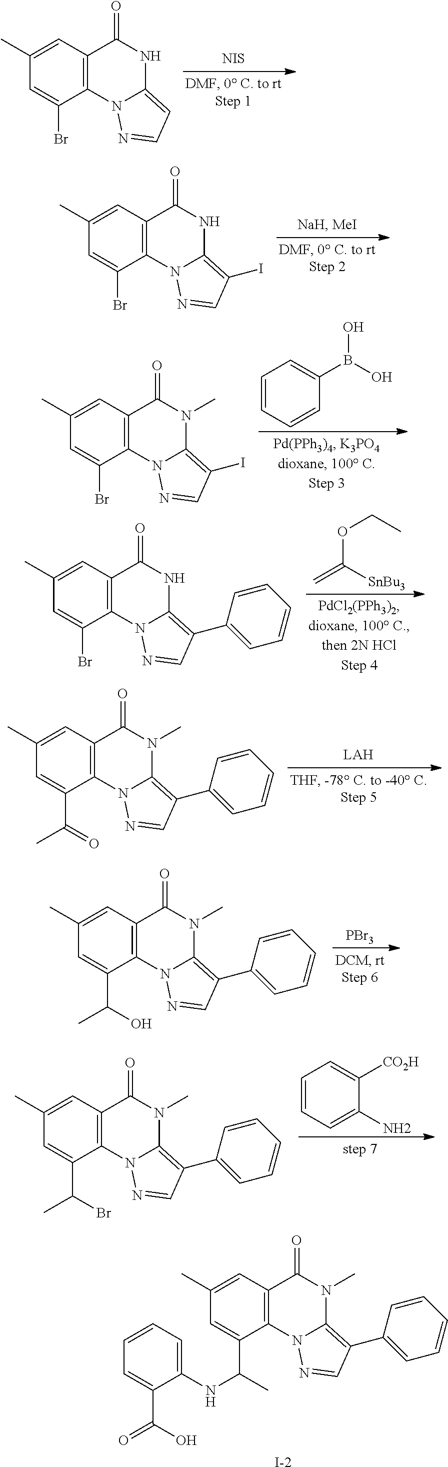

### Step 1: 9-bromo-3-iodo-7-methylpyrazolo[1,5-a]quinazolin-5(4H)-one

To a mixture of 9-bromo-7-methylpyrazolo[1,5-a]quinazolin-5(4H)-one (Example 1, step 2) (640 mg, 2.3 mmol) in DMF (20 mL) was added NIS (517 mg, 2.3 mmol) at 0° C. under nitrogen atmosphere. The reaction mixture was then slowly warmed to rt and stirred for 1 h. After completion, the reaction was diluted with ice water while stirred. The resulting solid was collected to afford pure product as a light brown solid (723 mg, 78%). LCMS calculated for C₁₁H₈BrIN₃O (M+H)⁺ m/z=404.0; found 404.1.

### Step 2: 9-bromo-3-iodo-4,7-dimethylpyrazolo[1,5-a]quinazolin-5(4H)-one

To a mixture of 9-bromo-3-iodo-7-methylpyrazolo[1,5-a]quinazolin-5(4H)-one (442 mg, 1.10 mmol) in DMF (20 mL) was added NaH (47 mg, 1.1 mmol) at 0° C. under nitrogen atmosphere. The resulting mixture was stirred at same temperature for 30 min MeI (200 mg, 1.3 mmol) was added. The reaction mixture was then slowly warmed to rt and stirred for 4 h. After completion, the reaction was carefully quenched with water at 0° C. while vigorously stirred. The mixture was then extracted with ethyl acetate (2×300 mL). The combined organics were dried with anhydrous sodium sulfate, filtered, and concentrated under reduced pressure. The residue was purified by silica gel column chromatography, eluted with hexanes and ethyl acetate to provide the desired product, which was further purified by silica gel column chromatography, eluted with DCM and ethyl acetate to provide the desired product as a white solid (332 mg, 72%). LCMS calculated for C₁₂H₁₀BrIN₃O (M+H)⁺ m/z=418.0; found 418.0.

### Step 3: 9-bromo-4,7-dimethyl-3-phenylpyrazolo[1,5-a]quinazolin-5(4H)-one

The mixture of 9-bromo-3-iodo-4,7-dimethylpyrazolo[1,5-a]quinazolin-5(4H)-one (200 mg, 0.48 mmol), Pd(PPh₃)₄ (57 mg, 0.05 mmol), phenylboronic acid (61 mg, 0.5 mmol), and K₃PO₄ (212 mg, 1 mmol) in dioxane (6 mL) and water (1 mL) was heated at 85° C. for 6 h under nitrogen atmosphere. Upon cooling to room temperature, the mixture was concentrated under reduced pressure. The residue was purified by silica gel column chromatography, eluted with DCM and ethyl acetate to provide the desired product as a brown solid (88 mg, 50%). LCMS calculated for C₁₈H₁₅BrN₃O (M+H)⁺ m/z=368.1; found 368.1.

### Step 4: 9-acetyl-4,7-dimethyl-3-phenylpyrazolo[1,5-a]quinazolin-5(4H)-one

The mixture of 9-bromo-4,7-dimethyl-3-phenylpyrazolo[1,5-a]quinazolin-5(4H)-one (88 mg, 0.24 mmol), PdCl₂(PPh₃)₂ (20 mg, 0.03 mmol), and tributyl(1-ethoxyvinyl)stannane (80 mg, 0.24 mmol) in dioxane (2 mL) was heated at 100° C. for 12 h under nitrogen atmosphere. Upon cooling to room temperature, the mixture was added 2 N HCl and stirred for 30 min. Then 200 mg CsF was added and stirred for another 30 min before the mixture was diluted with water and extracted with EtOAc. The combined organics were washed with sat. NaCl, dried with anhydrous sodium sulfate, filtered, and concentrated under reduced pressure. The residue was purified by silica gel column chromatography, eluted with DCM and ethyl acetate to provide the desired product as a brown oil (43 mg, 55%). LCMS calculated for C₂₀H₁₈N₃O₂ (M+H)⁺ m/z=332.1; found 332.1.

### Step 5: 9-(1-hydroxyethyl)-4,7-dimethyl-3-phenylpyrazolo[1,5-a]quinazolin-5(4H)-one

To a mixture of 9-acetyl-4,7-dimethyl-3-phenylpyrazolo[1,5-a]quinazolin-5(4H)-one (43 mg, 0.13 mmol) in THF (4 mL) was added LAH (0.2 mL, 0.2 mmol, 1 M solution in THF) at −78° C. The resulting mixture was stirred at same temperature for 10 min, and then warmed to −40° C. before quenched with sat. NH₄Cl. The mixture was diluted with water and extracted with EtOAc. The combined organics were washed with sat. NaCl, dried with anhydrous sodium sulfate, filtered, and concentrated under reduced pressure. The residue was purified by silica gel column chromatography, eluted with DCM and ethyl acetate to provide the desired product as a colorless oil (13 mg, 30%). LCMS calculated for C₂₀H₁₈N₃O (M−OH)⁺ m/z=316.1; found 316.1.

### Step 6: 9-(1-bromoethyl)-4,7-dimethyl-3-phenylpyrazolo[1,5-a]quinazolin-5(4H)-one

To a mixture of 9-(1-hydroxyethyl)-4,7-dimethyl-3-phenylpyrazolo[1,5-a]quinazolin-5(4H)-one (13 mg, 0.04 mmol) in DCM (1 mL) was added PBr₃ (20 mg, 0.7 mmol). The resulting mixture was stirred at rt for 2 h. The mixture was then quenched with sat. NaHCO₃ and extracted with DCM. The combined organics were washed with sat. NaCl, dried with anhydrous sodium sulfate, filtered, and concentrated under reduced pressure. The residue was used directly in next step without further purification.

### Step 7: 2-((1-(4,7-dimethyl-5-oxo-3-phenyl-4,5-dihydropyrazolo[1,5-a]quinazolin-9-yl)ethyl)amino)benzoic acid (I-2)

9-(1-bromoethyl)-4,7-dimethyl-3-phenylpyrazolo[1,5-a]quinazolin-5(4H)-one (14 mg, 0.035 mmol) in 2 dram vial was added DMF (0.3 mL), and 2-aminobenzoic acid (20 mg, 0.14 mmol). The mixture was heated to 90° C. for 2 h. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₂₅N₄O₃ (M+H)⁺ m/z=453.2; found 453.3.

### Example 3: 2-((1-(4,7-dimethyl-5-oxo-3-vinyl-4,5-dihydropyrazolo[1,5-a]quinazolin-9-yl)ethyl)amino)benzoic acid (I-3)

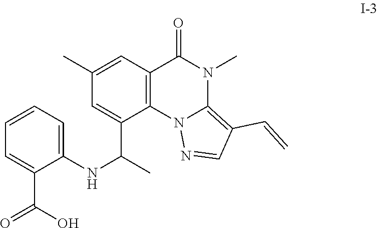 [SMILES: C=Cc1cnn2c3c(C(C)Nc4ccccc4C(=O)O)cc(C)cc3c(=O)n(C)c12]

The titled compound was prepared using similar procedures as described for Example 2 with 4,4,5,5-tetramethyl-2-vinyl-1,3,2-dioxaborolane replacing phenylboronic acid in Step 3. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₃N₄O₃ (M+H)+ m/z=403.2; found 403.2.

### Example 4: 2-((1-(4,7-Dimethyl-5-oxo-4,5-dihydroimidazo[1,5-a]quinazolin-9-yl)ethyl)amino)benzoic acid (I-4)

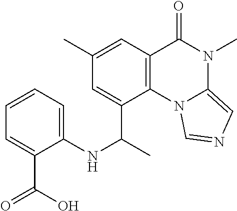 [SMILES: Cc1cc(C(C)Nc2ccccc2C(=O)O)c2c(c1)c(=O)n(C)c1cncn21]

### Step 1: 2-Amino-3-bromo-5-methylbenzoic acid

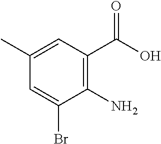

To a mixture of 2-amino-5-methylbenzoic acid (20 g, 132 mmol) in DMF (20 mL) was added NBS (23.4 g, 132 mmol) at 0° C. under nitrogen atmosphere. The reaction mixture was then slowly warmed to rt and stirred for 1 h. After completion, the reaction was diluted with ice water while stirred. The resulting solid was collected to afford the desired product as a light brown solid (25.2 g, 83%). LCMS calculated for C₈H₉BrNO₂ (M+H)⁺ m/z=230.0; found 230.1.

### Step 2: 8-Bromo-6-methyl-2H-benzo[d][1,3]oxazine-2,4(1H)-dione

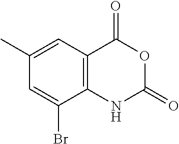

To a mixture of 2-amino-3-bromo-5-methylbenzoic acid (20 g, 87.3 mmol) in dioxane (300 mL) was added triphosgene (10.32 g, 34.9 mmol) at 0° C. The resulting mixture was stirred at 100° C. for 2 h. Upon cooling to room temperature, the resulting solid was collected to afford the desired product as a white solid, which was used in the next step without further purification.

### Step 3: 2-Amino-3-bromo-N,5-dimethylbenzamide

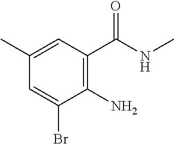

To a mixture of 8-bromo-6-methyl-2H-benzo[d][1,3]oxazine-2,4(1H)-dione (18 g, 70.8 mmol) in THF (300 mL) was added methylamine (35 mL, 2 M solution in THF) at 0° C. The resulting mixture was warmed to rt, and then stirred for 1 h. The solvent was removed, and crude product was used in next step without further purification.

### Step 4: 8-Bromo-2-hydroxy-3,6-dimethylquinazolin-4(3H)-one

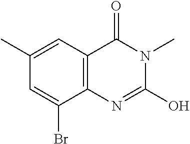

To the solid from Step 3 was added THF (300 mL) and triphosgene (10.32 g, 34.9 mmol) at rt. The resulting mixture was heated at 70° C. for 2 h, upon cooling to room temperature, half of the solvent was removed under vacuum, the resulting solid was collected. The solid product was then washed with 4:1 hexanes and ethyl acetate, then dried under vacuum overnight to afford pure product as a white solid, which was used in the next step without further purification.

### Step 5: 8-Bromo-2-chloro-3,6-dimethylquinazolin-4(3H)-one

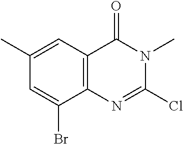

To a mixture of 8-bromo-2-hydroxy-3,6-dimethylquinazolin-4(3H)-one (16 g, 59.7 mmol) in POCl₃ (83 mL, 891.88 mmoL) was added DIEA (40.45 mL, 237.8 mmol) at 0° C. The resulting mixture was stirred at 120° C. for 12 h in a pressure vessel. Upon cooling to room temperature, the mixture was slowly poured onto 2000 mL ice carefully. After 2 h, the resulting solid was collected to afford a light brown solid, which was washed with water, sat NaHCO₃ then water. The solid was dried under vacuum to afford the desired product as light brown solid (13.6 g, 80%).

### Step 6: tert-Butyl 9-bromo-4,7-dimethyl-5-oxo-4,5-dihydroimidazo[1,5-a]quinazoline-3-carboxylate

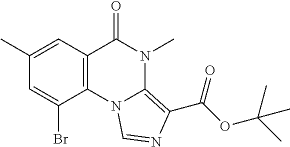 [SMILES: Cc1cc(Br)c2c(c1)c(=O)n(C)c1c(C(=O)OC(C)(C)C)ncn21]

To a mixture of KO^{t}Bu (2.9 g, 26.0 mmol) in dry DMF (100 mL) under nitrogen was added tert-butyl 2-isocyanoacetate (2.7 mL, 19.13 mmol) at 0° C. dropwise. The resulting mixture was stirred at 0° C. for 10 min before 8-bromo-2-chloro-3,6-dimethylquinazolin-4(3H)-one (5 g, 17.3 mmol) was added in one portion. The resulting mixture was warmed to rt and stirred for 2 h. After completion, the reaction was poured into 300 mL sat. NH₄Cl solution, then diluted with 200 mL water. The resulting solid was collected to afford pure product as a light brown solid, which was washed with water (2×). The solid was dried under vacuum to provide the desired product as beige solid (5.6 g, 85%). LCMS calculated for C₁₇H₁₉BrN₃O₃ (M+H)⁺ m/z=392.0; found 392.1.

### Step 7: tert-Butyl 9-acetyl-4,7-dimethyl-5-oxo-4,5-dihydroimidazo[1,5-a]quinazoline-3-carboxylate

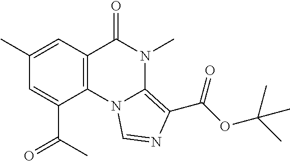 [SMILES: CC(=O)c1cc(C)cc2c(=O)n(C)c3c(C(=O)OC(C)(C)C)ncn3c12]

The mixture of tert-butyl 9-bromo-4,7-dimethyl-5-oxo-4,5-dihydroimidazo[1,5-a]quinazoline-3-carboxylate (5.2 g, 13.2 mmol), PdCl₂(PPh₃)₂ (930 mg, 1.33 mmol), and tributyl(1-ethoxyvinyl)stannane (5.7 g, 15.9 mmol) in dioxane (150 mL) was heated at 100° C. for 8 h under nitrogen atmosphere. Upon cooling to room temperature, the mixture was added 2 N HCl and stirred for 4 h. The mixture was diluted with water and extracted with EtOAc. The combined organics were washed with sat. NaCl, dried with anhydrous sodium sulfate, filtered, and concentrated under reduced pressure. The residue was purified by silica gel column chromatography, eluted with DCM and ethyl acetate to provide the desired product as a brown oil (4.1 g, 88%). LCMS calculated for C₁₉H₂₂N₃O₄ (M+H)⁺ m/z=356.2; found 356.2.

### Step 8: tert-Butyl 9-(1-hydroxyethyl)-4,7-dimethyl-5-oxo-4,5-dihydroimidazo[1,5-a]quinazoline-3-carboxylate

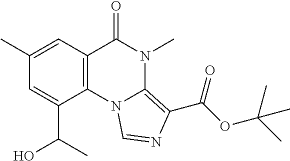 [SMILES: Cc1cc(C(C)O)c2c(c1)c(=O)n(C)c1c(C(=O)OC(C)(C)C)ncn21]

tert-Butyl 9-acetyl-4,7-dimethyl-5-oxo-4,5-dihydroimidazo[1,5-a]quinazoline-3-carboxylate (5 g, 14 mmol) was dissolved in MeOH (100 mL) and DCM (100 mL) then NaBH₄ (551 mg, 14.58 mmol) was added at 0° C. in three portions under nitrogen. The resulting mixture was stirred at same temperature for 10 min before quenched with sat. NH₄Cl. The mixture was diluted with water and extracted with DCM. The combined organics were washed with sat. NaCl, and dried with anhydrous sodium sulfate, filtered, and concentrated under reduced pressure. The residue was purified by silica gel column chromatography, eluted with DCM and MeOH (0 to 4%) to provide the desired product as a light yellow solid (3.5 g, 70%). LCMS calculated for C₁₉H₂₄N₃O₄ (M+H)⁺ m/z=358.2; found 358.2.

### Step 9: tert-Butyl 9-(1-bromoethyl)-4,7-dimethyl-5-oxo-4,5-dihydroimidazo[1,5-a]quinazoline-3-carboxylate

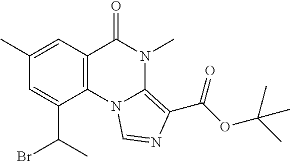 [SMILES: Cc1cc(C(C)Br)c2c(c1)c(=O)n(C)c1c(C(=O)OC(C)(C)C)ncn21]

To a mixture of tert-butyl 9-(1-hydroxyethyl)-4,7-dimethyl-5-oxo-4,5-dihydroimidazo[1,5-a]quinazoline-3-carboxylate (3.0 g, 8.4 mmol) in DCM (200 mL) was added PBr₃ (0.95 mL, 10 mmol) at 0° C. The resulting mixture was warmed to rt and stirred for 2 h. The mixture was then quenched with sat. NaHCO₃ and extracted with DCM (2×). The combined organics were washed with sat. NaCl, and dried with anhydrous sodium sulfate, filtered, and concentrated under reduced pressure. The residue was used directly in next step without further purification.

### Step 10: tert-Butyl 9-(1-((2-(methoxycarbonyl)phenyl)amino)ethyl)-4,7-dimethyl-5-oxo-4,5-dihydroimidazo[1,5-a]quinazoline-3-carboxylate

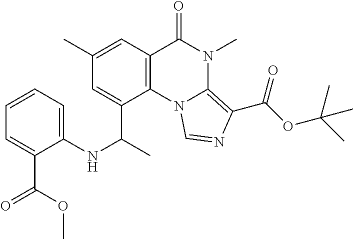 [SMILES: COC(=O)c1ccccc1NC(C)c1cc(C)cc2c(=O)n(C)c3c(C(=O)OC(C)(C)C)ncn3c12]

To the solution of tert-butyl 9-(1-bromoethyl)-4,7-dimethyl-5-oxo-4,5-dihydroimidazo[1,5-a]quinazoline-3-carboxylate (3.2 g, 7.6 mmol) in DMF (100 mL) was added methyl 2-aminobenzoate (4.0 g, 26 mmol). The mixture was heated to 80° C. for 2 h. Upon cooling to room temperature, the mixture was diluted with water and extracted with ethyl acetate (3×). The combined organics were washed with water, sat. NaCl, and dried with anhydrous sodium sulfate, filtered, and concentrated under reduced pressure. The residue was purified by silica gel column chromatography, eluted with hexanes and EtOAc (0 to 30%) to provide the desired product as a light yellow solid (2.7 g, 73%). LCMS calculated for C₂₇H₃₁N₄O₅ (M+H)⁺ m/z=491.2; found 491.2.

### Step 11: 9-(1-((2-(Methoxycarbonyl)phenyl)amino)ethyl)-4,7-dimethyl-5-oxo-4,5-dihydroimidazo[1,5-a]quinazoline-3-carboxylic acid

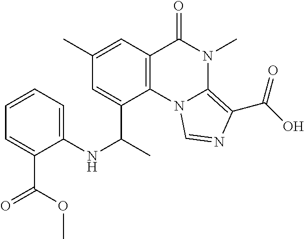 [SMILES: COC(=O)c1ccccc1NC(C)c1cc(C)cc2c(=O)n(C)c3c(C(=O)O)ncn3c12]

tert-Butyl 9-(1-((2-(methoxycarbonyl)phenyl)amino)ethyl)-4,7-dimethyl-5-oxo-4,5-dihydroimidazo[1,5-a]quinazoline-3-carboxylate (1.7 g, 3.4 mmol) was dissolved in TFA (20 mL). The mixture was stirred for 3 h at rt. After completion, TFA was removed under vacuum. 200 mL of ice water and 50 mL of EtOAc were added to the reaction residue while stirring. After stirring for 20 min, the resulting solid was collected to afford a light brown solid, which was washed with water (2×). The solid was dried under vacuum to provide the pure product (1.2 g, 81%). LCMS calculated for C₂₃H₂₃N₄O₅ (M+H)⁺ m/z=435.2; found 435.2.

### Step 12: Methyl 2-((1-(3-iodo-4,7-dimethyl-5-oxo-4,5-dihydroimidazo[1,5-a]quinazolin-9-yl)ethyl)amino)benzoate

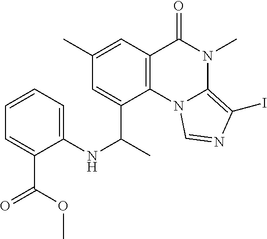 [SMILES: COC(=O)c1ccccc1NC(C)c1cc(C)cc2c(=O)n(C)c3c(I)ncn3c12]

To a mixture of 9-(1-((2-(methoxycarbonyl)phenyl)amino)ethyl)-4,7-dimethyl-5-oxo-4,5-dihydroimidazo[1,5-a]quinazoline-3-carboxylic acid (1.2 g, 2.76 mmol) in DMF (20 mL) under nitrogen was added NaHCO₃ (928 mg, 11.05 mmol) at 0° C., after stirring for 5 min, NIS (745 mg, 3.31 mmol) was added in one portion. The resulting mixture was warmed to rt and stirred for 2 h. After completion, the reaction was quenched with sat. Na₂S₂O₃ and diluted with water. The mixture was then extracted with EtOAc (3×). The combined organics were washed with water, sat. NaCl, and dried with anhydrous sodium sulfate, filtered, and concentrated under reduced pressure. The residue was purified by silica gel column chromatography, eluted with hexanes and EtOAc (0 to 30%) to provide the desired product as a white solid (1.1 g, 74%). LCMS calculated for C₂₂H₂₂IN₄O₃ (M+H)⁺ m/z=517.1; found 517.1.

### Step 13: Methyl 2-((1-(4,7-dimethyl-5-oxo-4,5-dihydroimidazo[1,5-a]quinazolin-9-yl)ethyl)amino)benzoate

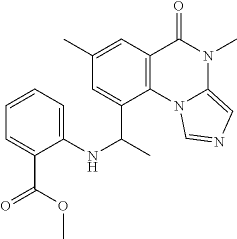 [SMILES: COC(=O)c1ccccc1NC(C)c1cc(C)cc2c(=O)n(C)c3cncn3c12]

In a 2 mL drum vial, methyl 2-((1-(3-iodo-4,7-dimethyl-5-oxo-4,5-dihydroimidazo[1,5-a]quinazolin-9-yl)ethyl)amino)benzoate (20 mg, 0.04 mmol) and Pd/C (10 wt % on active carbon, 10 mg) was dissolved in MeOH (2 mL). The vial was then charged with 1 atm H₂ and stirred for 2 h. The reaction mixture was then filtrate through a short pad of celite and solvent was removed under vacuum to afford the crude product which was used in next step without further purification.

### Step 14: 2-((1-(4,7-Dimethyl-5-oxo-4,5-dihydroimidazo[1,5-a]quinazolin-9-yl)ethyl)amino)benzoic acid

To a mixture of methyl 2-((1-(4,7-dimethyl-5-oxo-4,5-dihydroimidazo[1,5-a]quinazolin-9-yl)ethyl)amino)benzoate (12 mg, 0.03 mmol) in THF (0.2 mL) and MeOH (0.5 mL) was added 0.5 mL of 2N NaOH solution. The mixture was heated at 75° C. for 20 min before neutralized by adding 2N HCl solution. The mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₁H₂₁N₄O₃ (M+H)⁺ m/z=377.2; found 377.2.

### Example 5: 2-((1-(3-Ethyl-4,7-dimethyl-5-oxo-4,5-dihydroimidazo[1,5-a]quinazolin-9-yl)ethyl)amino)benzoic acid (I-5)

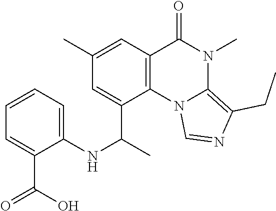 [SMILES: CCc1ncn2c3c(C(C)Nc4ccccc4C(=O)O)cc(C)cc3c(=O)n(C)c12]

### Step 1: Methyl 2-((1-(4,7-dimethyl-5-oxo-3-vinyl-4,5-dihydroimidazo[1,5-a]quinazolin-9-yl)ethyl)amino)benzoate

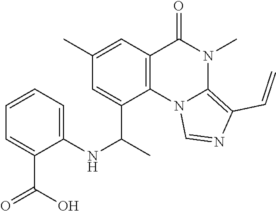 [SMILES: C=Cc1ncn2c3c(C(C)Nc4ccccc4C(=O)O)cc(C)cc3c(=O)n(C)c12]

A mixture of methyl 2-((1-(3-iodo-4,7-dimethyl-5-oxo-4,5-dihydroimidazo[1,5-a]quinazolin-9-yl)ethyl)amino)benzoate (Example 4, Step 12:40 mg, 0.08 mmol), XphosG2-Pd (7 mg, 0.01 mmol), 4,4,5,5-tetramethyl-2-vinyl-1,3,2-dioxaborolane (15 mg, 0.1 mmol), and K₃PO₄ (21 mg, 0.1 mmol) in dioxane (1 mL) and water (0.1 mL) was heated at 100° C. for 4 h under nitrogen atmosphere. Upon cooling to room temperature, the mixture was concentrated under reduced pressure. The residue was purified by silica gel column chromatography, eluted with hexanes and ethyl acetate to provide the desired product as a brown solid (23 mg, 71%). LCMS calculated for C₂₄H₂₅N₄O₃ (M+H)⁺ m/z=417.2; found 417.2.

### Step 2: Methyl 2-((1-(3-ethyl-4,7-dimethyl-5-oxo-4,5-dihydroimidazo[1,5-a]quinazolin-9-yl)ethyl)amino)benzoate

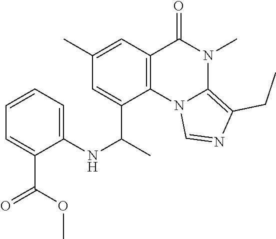 [SMILES: CCc1ncn2c3c(C(C)Nc4ccccc4C(=O)OC)cc(C)cc3c(=O)n(C)c12]

In a 2 mL drum vial, methyl 2-((1-(4,7-dimethyl-5-oxo-3-vinyl-4,5-dihydroimidazo[1,5-a]quinazolin-9-yl)ethyl)amino)benzoate (23 mg, 0.06 mmol) and Pd/C (10 wt % on active carbon, 7 mg) was dissolved in MeOH (2 mL). The vial was then charged with 1 atm H₂ and stirred for 2 h. The reaction mixture was then filtrate through a short pad of celite and solvent was removed under vacuum to afford the crude product which was used in next step without further purification.

### Step 3: 2-((1-(3-Ethyl-4,7-dimethyl-5-oxo-4,5-dihydroimidazo[1,5-a]quinazolin-9-yl)ethyl)amino)benzoic acid

To a mixture of methyl 2-((1-(3-ethyl-4,7-dimethyl-5-oxo-4,5-dihydroimidazo[1,5-a]quinazolin-9-yl)ethyl)amino)benzoate (17 mg, 0.04 mmol) in THF (0.2 mL) and MeOH (0.5 mL) was added 0.5 mL of 2N NaOH solution. The mixture was heated at 75° C. for 20 min before neutralized by adding 2N HCl solution. The mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₅N₄O₃ (M+H)⁺ m/z=405.2; found 405.2.

### Example 6: 2-((1-(4,7-Dimethyl-5-oxo-3-(pyridin-2-yl)-4,5-dihydroimidazo[1,5-a]quinazolin-9-yl)ethyl)amino)benzoic acid (I-6)

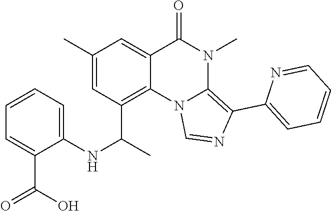 [SMILES: Cc1cc(C(C)Nc2ccccc2C(=O)O)c2c(c1)c(=O)n(C)c1c(-c3ccccn3)ncn21]

### Step 1: Methyl 2-((1-(4,7-dimethyl-5-oxo-3-(pyridin-2-yl)-4,5-dihydroimidazo[1,5-a]quinazolin-9-yl)ethyl)amino)benzoate

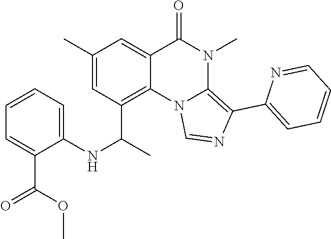 [SMILES: COC(=O)c1ccccc1NC(C)c1cc(C)cc2c(=O)n(C)c3c(-c4ccccn4)ncn3c12]

A mixture of methyl 2-((1-(3-iodo-4,7-dimethyl-5-oxo-4,5-dihydroimidazo[1,5-a]quinazolin-9-yl)ethyl)amino)benzoate (Example 4, Step 12:20 mg, 0.04 mmol), Pd(PPh₃)₄ (11 mg, 0.01 mmol), 2-(tributylstannyl)pyridine (29 mg, 0.08 mmol), CuI (2 mg, 0.01 mmol) and CsF (24 mg, 0.16 mmol) in dioxane (1 mL) was heated at 100° C. for 8 h under nitrogen atmosphere. Upon cooling to room temperature, the mixture was concentrated under reduced pressure. The residue was purified by silica gel column chromatography, eluted with hexanes and ethyl acetate to provide the desired product as a brown solid (11 mg, 61%). LCMS calculated for C₂₇H₂₆N₅O₃ (M+H)⁺ m/z=468.2; found 468.2.

### Step 2: 2-((1-(4,7-Dimethyl-5-oxo-3-(pyridin-2-yl)-4,5-dihydroimidazo[1,5-a]quinazolin-9-yl)ethyl)amino)benzoic acid

To a mixture of methyl 2-((1-(4,7-dimethyl-5-oxo-3-(pyridin-2-yl)-4,5-dihydroimidazo[1,5-a]quinazolin-9-yl)ethyl)amino)benzoate (11 mg, 0.024 mmol) in THF (0.2 mL) and MeOH (0.5 mL) was added 0.5 mL of 2N NaOH solution. The mixture was heated at 75° C. for 20 min before 0.7 mL of 2N HCl solution was added. The mixture was heated at 75° C. for 20 min before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₂₄N₅O₃ (M+H)⁺ m/z=454.2; found 454.2.

### Example 7: 2-((1-(3,4,7-Trimethyl-5-oxo-4,5-dihydroimidazo[1,5-a]quinazolin-9-yl)ethyl)amino)benzoic acid (I-7)

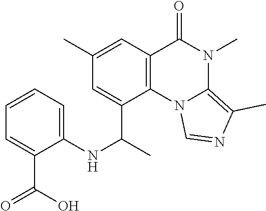 [SMILES: Cc1cc(C(C)Nc2ccccc2C(=O)O)c2c(c1)c(=O)n(C)c1c(C)ncn21]

The titled compound was prepared using similar procedures as described for Example 5 with methylboronic acid replacing 4,4,5,5-tetramethyl-2-vinyl-1,3,2-dioxaborolane in Step 1. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₂H₂₃N₄O₃ (M+H)⁺ m/z=391.2; found 391.2.

### Example 8: 6-Methyl-3-((1-(3,4,7-trimethyl-5-oxo-4,5-dihydroimidazo[1,5-a]quinazolin-9-yl)ethyl)amino)picolinic acid (I-8)

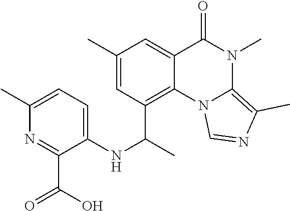

### Step 1: Methyl 3-((1-(3-iodo-4,7-dimethyl-5-oxo-4,5-dihydroimidazo[1,5-a]quinazolin-9-yl)ethyl)amino)-6-methylpicolinate

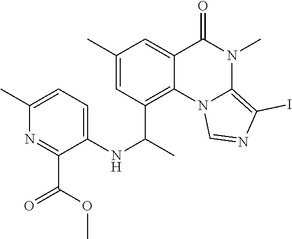 [SMILES: COC(=O)c1nc(C)ccc1NC(C)c1cc(C)cc2c(=O)n(C)c3c(I)ncn3c12]

The titled compound was prepared using similar procedures as described for Example 4 with methyl 3-amino-6-methylpicolinate replacing methyl 2-aminobenzoate in Step 10. The product was purified by silica gel column chromatography, eluted with DCM and MeOH to provide the desired product as a brown solid. LCMS calculated for C₂₂H₂₃IN₅O₃ (M+H)⁺ m/z=532.1; found 532.1.

### Step 2: Methyl 6-methyl-3-((1-(3,4,7-trimethyl-5-oxo-4,5-dihydroimidazo[1,5-a]quinazolin-9-yl)ethyl)amino)picolinate

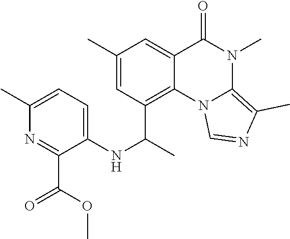

A mixture of methyl 3-((1-(3-iodo-4,7-dimethyl-5-oxo-4,5-dihydroimidazo[1,5-a]quinazolin-9-yl)ethyl)amino)-6-methylpicolinate (40 mg, 0.08 mmol), XphosG2-Pd (7 mg, 0.01 mmol), methylboronic acid (18 mg, 0.3 mmol), and K₃PO₄ (40 mg, 0.2 mmol) in dioxane (1 mL) and water (0.1 mL) was heated at 100° C. for 4 h under nitrogen atmosphere. Upon cooling to room temperature, the mixture was concentrated under reduced pressure. The residue was purified by silica gel column chromatography, eluted with hexanes and ethyl acetate to provide the desired product as a brown solid (19 mg, 60%). LCMS calculated for C₂₃H₂₆N₅O₃ (M+H)⁺ m/z=420.2; found 420.2.

### Step 3: 6-Methyl-3-((1-(3,4,7-trimethyl-5-oxo-4,5-dihydroimidazo[1,5-a]quinazolin-9-yl)ethyl)amino)picolinic acid

To a mixture of methyl 6-methyl-3-((1-(3,4,7-trimethyl-5-oxo-4,5-dihydroimidazo[1,5-a]quinazolin-9-yl)ethyl)amino)picolinate (10 mg, 0.023 mmol) in THF (0.2 mL) and MeOH (0.5 mL) was added 0.5 mL of 2N NaOH solution. The mixture was heated at 75° C. for 20 min before neutralized by adding 2N HCl solution. The mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₂H₂₄N₅O₃ (M+H)⁺ m/z=406.2; found 406.2.

### Example 9: 2-((1-(7-Chloro-3-ethyl-4-methyl-5-oxo-4,5-dihydroimidazo[1,5-a]quinazolin-9-yl)ethyl)amino)benzoic acid (I-9)

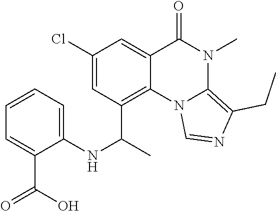 [SMILES: CCc1ncn2c3c(C(C)Nc4ccccc4C(=O)O)cc(Cl)cc3c(=O)n(C)c12]

The titled compound was prepared using similar procedures as described for Example 5 with methyl 2-((1-(7-chloro-3-iodo-4-methyl-5-oxo-4,5-dihydroimidazo[1,5-a]quinazolin-9-yl)ethyl)amino)benzoate (prepared using similar procedures as described for Example 4) replacing methyl 2-((1-(3-iodo-4,7-dimethyl-5-oxo-4,5-dihydroimidazo[1,5-a]quinazolin-9-yl)ethyl)amino)benzoate in Step 1. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₂H₂₂ClN₄O₃ (M+H)⁺ m/z=425.1; found 425.2.

### Example 10: 2-((1-(7-Chloro-4-methyl-5-oxo-4,5-dihydroimidazo[1,5-a]quinazolin-9-yl)ethyl)amino)benzoic acid (I-10)

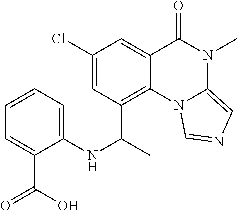 [SMILES: CC(Nc1ccccc1C(=O)O)c1cc(Cl)cc2c(=O)n(C)c3cncn3c12]

The titled compound was prepared using similar procedures as described for Example 4 with methyl 2-amino-5-chlorobenzoic acid replacing 2-amino-5-methylbenzoic acid in Step 1. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₀H₁₈ClN₄O₃ (M+H)⁺ m/z=397.1; found 397.1.

### Example 11: 2-((1-(7-Chloro-3,4-dimethyl-5-oxo-4,5-dihydroimidazo[1,5-a]quinazolin-9-yl)ethyl)amino)benzoic acid (I-11)

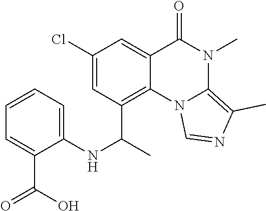 [SMILES: Cc1ncn2c3c(C(C)Nc4ccccc4C(=O)O)cc(Cl)cc3c(=O)n(C)c12]

The titled compound was prepared using similar procedures as described for Example 5 with methylboronic acid replacing 4,4,5,5-tetramethyl-2-vinyl-1,3,2-dioxaborolane, and methyl 2-((1-(7-chloro-3-iodo-4-methyl-5-oxo-4,5-dihydroimidazo[1,5-a]quinazolin-9-yl)ethyl)amino)benzoate replacing methyl 2-((1-(3-iodo-4,7-dimethyl-5-oxo-4,5-dihydroimidazo[1,5-a]quinazolin-9-yl)ethyl)amino)benzoate in Step 1. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₁H₂₀ClN₄O₃ (M+H)⁺ m/z=411.1; found 411.1.

### Example 12: 2-((1-(7-Chloro-4-ethyl-5-oxo-4,5-dihydroimidazo[1,5-a]quinazolin-9-yl)ethyl)amino)benzoic acid (I-12)

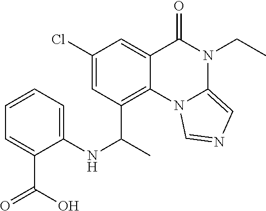 [SMILES: CCn1c(=O)c2cc(Cl)cc(C(C)Nc3ccccc3C(=O)O)c2n2cncc12]

The titled compound was prepared using similar procedures as described for Example 4 with 2-amino-5-chlorobenzoic acid replacing 2-amino-5-methylbenzoic acid in Step 1 and ethyl amine replacing methylamine in Step 3. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₁H₂₀ClN₄O₃ (M+H)⁺ m/z=411.1; found 411.2.

### Example 13: 2-((1-(7-Chloro-3,4-diethyl-5-oxo-4,5-dihydroimidazo[1,5-a]quinazolin-9-yl)ethyl)amino)benzoic acid (I-13)

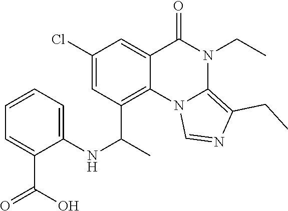 [SMILES: CCc1ncn2c3c(C(C)Nc4ccccc4C(=O)O)cc(Cl)cc3c(=O)n(CC)c12]

The titled compound was prepared using similar procedures as described for Example 5 with methyl 2-((1-(7-chloro-4-ethyl-3-iodo-5-oxo-4,5-dihydroimidazo[1,5-a]quinazolin-9-yl)ethyl)amino)benzoate replacing methyl 2-((1-(3-iodo-4,7-dimethyl-5-oxo-4,5-dihydroimidazo[1,5-a]quinazolin-9-yl)ethyl)amino)benzoate in Step 1. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₄ClN₄O₃ (M+H)⁺ m/z=439.2; found 439.2.

### Example 14: 2-((1-(4,7-Dimethyl-5-oxo-4,5-dihydroimidazo[1,2-a]quinazolin-9-yl)ethyl)amino)benzoic acid (I-14)

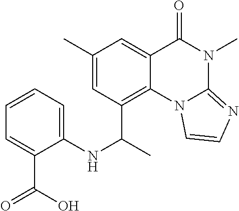 [SMILES: Cc1cc(C(C)Nc2ccccc2C(=O)O)c2c(c1)c(=O)n(C)c1nccn21]

### Step 1: 9-Bromo-4,7-dimethylimidazo[1,2-a]quinazolin-5(4H)-one

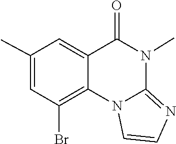

To a mixture of 8-bromo-2-chloro-3,6-dimethylquinazolin-4(3H)-one (Example 4, Step 5:200 mg, 0.7 mmol) in DMF (1 mL) was added 2,2-dimethoxyethan-1-amine (219 mg, 2.1 mmol) under nitrogen atmosphere. The reaction mixture was heated at 145° C. for 12 h. After completion, the reaction was diluted with water and extracted with EtOAc. The combined organics were washed with water, sat. NaCl, and dried with anhydrous sodium sulfate, filtered, and concentrated under reduced pressure. The residue was purified by silica gel column chromatography, eluted with DCM and MeOH (0 to 10%) to provide the desired product as a light yellow solid (85 mg, 42%). LCMS calculated for C₁₂H₁₁BrN₃O (M+H)⁺ m/z=292.0; found 292.0.

### Step 2: 9-Acetyl-4,7-dimethylimidazo[1,2-a]quinazolin-5(4H)-one

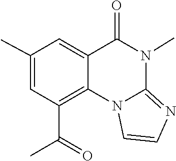

A mixture of 9-bromo-4,7-dimethylimidazo[1,2-a]quinazolin-5(4H)-one (85 mg, 0.29 mmol), PdCl₂(PPh₃)₂ (14 mg, 0.02 mmol), and tributyl(1-ethoxyvinyl)stannane (108 mg, 0.3 mmol) in dioxane (2 mL) was heated at 100° C. for 8 h under nitrogen atmosphere. Upon cooling to room temperature, the mixture was added 2 N HCl and stirred for 4 h. The mixture was diluted with water and extracted with EtOAc. The combined organics were washed with sat. NaCl, dried with anhydrous sodium sulfate, filtered, and concentrated under reduced pressure. The residue was purified by silica gel column chromatography, eluted with DCM and MeOH to provide the desired product as a brown oil (59 mg, 80%). LCMS calculated for C₁₄H₁₄N₃O₂ (M+H)⁺ m/z=256.1; found 256.2.

### Step 3: 9-(1-Hydroxyethyl)-4,7-dimethylimidazo[1,2-a]quinazolin-5(4H)-one

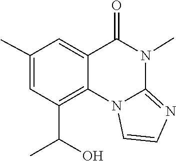

9-Acetyl-4,7-dimethylimidazo[1,2-a]quinazolin-5(4H)-one (59 mg, 0.23 mmol) was dissolved in THF (4 mL) and cooled down to −40° C. LAH (1M THF solution, 0.23 mL) was added at dropwise. The resulting mixture was stirred at same temperature for 10 min before quenched with sat. NH₄Cl. The mixture was diluted with water and extracted with EtOAc. The combined organics were washed with sat. NaCl, and dried with anhydrous sodium sulfate, filtered, and concentrated under reduced pressure. The residue was purified by silica gel column chromatography, eluted with DCM and MeOH (0 to 20%) to provide the desired product as a light yellow solid (23 mg 40%). LCMS calculated for C₁₄H₁₆N₃O₂ (M+H)⁺ m/z=258.1; found 258.1.

### Step 4: 9-(1-Bromoethyl)-4,7-dimethylimidazo[1,2-a]quinazolin-5(4H)-one

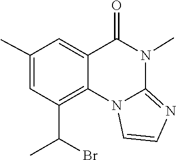

To a mixture of 9-(1-hydroxyethyl)-4,7-dimethylimidazo[1,2-a]quinazolin-5(4H)-one (23 mg, 0.09 mmol) in DCM (2 mL) was added PBr₃ (56 mg, 0.2 mmol) at 0° C. The resulting mixture was warmed to rt and stirred for 2 h. The mixture was then quenched with sat. NaHCO₃ and extracted with DCM (2×). The combined organics were washed with sat. NaCl, and dried with anhydrous sodium sulfate, filtered, and concentrated under reduced pressure. The residue was used directly in next step without further purification.

### Step 5: 2-((1-(4,7-Dimethyl-5-oxo-4,5-dihydroimidazo[1,2-a]quinazolin-9-yl)ethyl)amino)benzoic acid

To a solution of 9-(1-bromoethyl)-4,7-dimethylimidazo[1,2-a]quinazolin-5(4H)-one (12 mg, 0.04 mmol) in DMF (0.5 mL) was added 2-aminobenzoic acid (30 mg, 0.22 mmol). The mixture was heated to 80° C. for 2 h. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₁H₂₁N₄O₃ (M+H)⁺ m/z=377.2;
found 377.2.

### Example 15: 2-((1-(4,7-Dimethyl-5-oxo-4,5-dihydro-[1,2,4]triazolo[1,5-a]quinazolin-9-yl)ethyl)amino)benzoic acid (I-15)

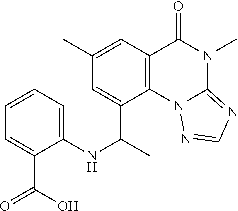 [SMILES: Cc1cc(C(C)Nc2ccccc2C(=O)O)c2c(c1)c(=O)n(C)c1ncnn21]

The titled compound was prepared using similar procedures as described for Example 1 with 1H-1,2,4-triazol-5-amine replacing 1H-pyrazol-5-amine in Step 2. The product was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₀H₂₀N₅O₃ (M+H)⁺ m/z=378.2; found 378.2.

### Example 16: 2-((1-(3,4,7-Trimethyl-5-oxo-4,5-dihydro-3H-pyrazolo[3,4-c]isoquinolin-9-yl)ethyl)amino)benzoic acid (I-16)

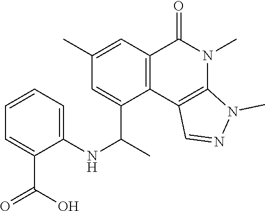 [SMILES: Cc1cc(C(C)Nc2ccccc2C(=O)O)c2c(c1)c(=O)n(C)c1c2cnn1C]

### Step 1: 3-Bromo-2-iodo-5-methyl-N-(1-methyl-1H-pyrazol-5-yl)benzamide

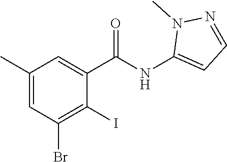 [SMILES: Cc1cc(Br)c(I)c(C(=O)Nc2ccnn2C)c1]

To a mixture of 3-bromo-2-iodo-5-methylbenzoic acid (1.9 g, 5.7 mmol) in DMF (10 mL) was added HATU (2.35 g, 6.2 mmol), 1-methyl-1H-pyrazol-5-amine (500 mg, 5.2 mmol) and DIPEA (2.7 mL, 15.5 mmol). The reaction was stirred at r.t. for 4 h. After completion, the reaction was quenched with water. The mixture was then extracted with ethyl acetate (2×300 mL). The combined organics were dried with anhydrous sodium sulfate, filtered, and concentrated under reduced pressure, which was further purified by silica gel column chromatography, eluted with DCM and ethyl acetate to provide the desired product as white solid (1.8 g, 83%). LCMS calculated for C₁₂H₁₂BrIN₃O (M+H)⁺ m/z=419.9; found 419.9.

### Step 2: 3-Bromo-2-iodo-N,5-dimethyl-N-(1-methyl-1H-pyrazol-5-yl)benzamide

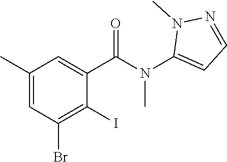 [SMILES: Cc1cc(Br)c(I)c(C(=O)N(C)c2ccnn2C)c1]

To a mixture of 3-bromo-2-iodo-5-methyl-N-(1-methyl-1H-pyrazol-5-yl)benzamide (459 mg, 1.10 mmol) in DMF (20 mL) was added NaH (47 mg, 1.1 mmol) at 0° C. under nitrogen atmosphere. The resulting mixture was stirred at same temperature for 30 min and then MeI (200 mg, 1.3 mmol) was added. The reaction mixture was then slowly warmed to rt and stirred for 1 h. After completion, the reaction was carefully quenched with water at 0° C. while vigorously stirred. The mixture was then extracted with ethyl acetate (2×300 mL). The combined organics were dried with anhydrous sodium sulfate, filtered, and concentrated under reduced pressure. The residue was purified by silica gel column chromatography, eluted with hexanes and ethyl acetate to provide the desired product, which was further purified by silica gel column chromatography, eluted with DCM and ethyl acetate to provide the desired product as a white solid (370 mg, 78%). LCMS calculated for C₁₃H₁₄BrIN₃O (M+H)⁺ m/z=433.9; found 434.0.

### Step 3: 9-Bromo-3,4,7-trimethyl-3,4-dihydro-5H-pyrazolo[3,4-c]isoquinolin-5-one

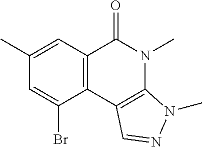

A mixture of 3-bromo-2-iodo-N,5-dimethyl-N-(1-methyl-1H-pyrazol-5-yl)benzamide (148 mg, 0.34 mmol), Pd(PPh₃)₄ (196 mg, 0.17 mmol), KOAc (100 mg, 1 mmol) in DMAc (2 mL) was heated at 120° C. for 16 h under nitrogen atmosphere. Upon cooling to room temperature, the mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a white solid (57 mg, 54%). LCMS calculated for C₁₃H₁₃BrN₃O (M+H)⁺ m/z=306.0; found 306.0.

### Step 4: 9-Acetyl-3,4,7-trimethyl-3,4-dihydro-5H-pyrazolo[3,4-c]isoquinolin-5-one

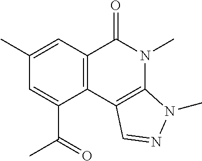

A mixture of 9-bromo-3,4,7-trimethyl-3,4-dihydro-5H-pyrazolo[3,4-c]isoquinolin-5-one (100 mg, 0.33 mmol), PdCl₂(PPh₃)₂ (23 mg, 0.03 mmol), and tributyl(1-ethoxyvinyl)stannane (143 mg, 0.4 mmol) in dioxane (2 mL) was heated at 100° C. for 2 h under nitrogen atmosphere. Upon cooling to room temperature, the mixture was added 2 N HCl and stirred for 30 min. Then 200 mg CsF was added and stirred for another 30 min before the mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a white solid (59 mg, 67%). LCMS calculated for C₁₅H₁₆N₃O₂ (M+H)⁺ m/z=270.1; found 270.1.

### Step 5: 9-(1-Hydroxyethyl)-3,4,7-trimethyl-3,4-dihydro-5H-pyrazolo[3,4-c]isoquinolin-5-one

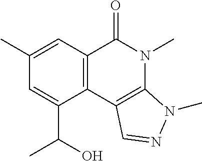

To a mixture of 9-acetyl-3,4,7-trimethyl-3,4-dihydro-5H-pyrazolo[3,4-c]isoquinolin-5-one (60 mg, 0.22 mmol) in THF (2 mL) was added LAH (0.3 mL, 0.3 mmol, 1 M solution in THF) at −78° C. The resulting mixture was stirred at same temperature for 10 min, and then warmed to −40° C. before quenched with sat. NH₄Cl. The mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a white solid (55 mg, 92%). LCMS calculated for C₁₅H₁₈N₃O₂ (M+H)⁺ m/z=272.1; found 272.1.

### Step 6: 9-(1-Bromoethyl)-3,4,7-trimethyl-3,4-dihydro-5H-pyrazolo[3,4-c]isoquinolin-5-one

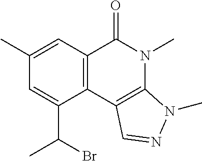

To a mixture of 9-(1-hydroxyethyl)-3,4,7-trimethyl-3,4-dihydro-5H-pyrazolo[3,4-c]isoquinolin-5-one (20 mg, 0.07 mmol) in DCM (1 mL) was added PBr₃ (38 mg, 0.14 mmol). The resulting mixture was stirred at rt for 2 h. The mixture was then quenched with sat. NaHCO₃ and extracted with DCM. The combined organics were washed with sat. NaCl, dried with anhydrous sodium sulfate, filtered, and concentrated under reduced pressure. The residue was used directly in next step without further purification.

### Step 7: 2-((1-(3,4,7-Trimethyl-5-oxo-4,5-dihydro-3H-pyrazolo[3,4-c]isoquinolin-9-yl)ethyl)amino)benzoic acid

9-(1-Bromoethyl)-3,4,7-trimethyl-3,4-dihydro-5H-pyrazolo[3,4-c]isoquinolin-5-one (20 mg, 0.06 mmol) in 2 dram vial was added DMF (0.3 mL), and 2-aminobenzoic acid (20 mg, 0.14 mmol). The mixture was heated to 90° C. for 2 h. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₂H₂₃N₄O₃ (M+H)⁺ m/z=391.2; found 391.2.

### Example 17: 6-Chloro-3-((1-(3,4,7-trimethyl-5-oxo-4,5-dihydro-3H-pyrazolo[3,4-c]isoquinolin-9-yl)ethyl)amino)picolinic acid (I-17)

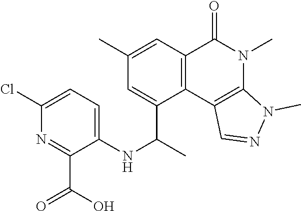 [SMILES: Cc1cc(C(C)Nc2ccc(Cl)nc2C(=O)O)c2c(c1)c(=O)n(C)c1c2cnn1C]

The titled compound was prepared using similar procedures as described for Example 16 with 3-amino-6-chloropyridine-2-carboxylic acid replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₁H₂₁ClN₅O₃ (M+H)⁺ m/z=426.1; found 426.1.

### Example 18: 2-((1-(3-Ethyl-4,7-dimethyl-5-oxo-4,5-dihydro-3H-pyrazolo[3,4-c]isoquinolin-9-yl)ethyl)amino)benzoic acid (I-18)

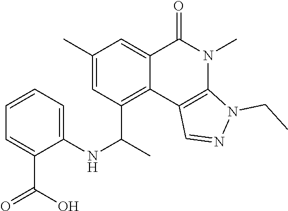 [SMILES: CCn1ncc2c3c(C(C)Nc4ccccc4C(=O)O)cc(C)cc3c(=O)n(C)c21]

The titled compound was prepared using similar procedures as described for Example 16 with 1-ethyl-1H-pyrazol-5-amine replacing 1-methyl-1H-pyrazol-5-amine in Step 1. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₅N₄O₃ (M+H)⁺ m/z=405.2; found 405.2.

### Example 19: 6-Chloro-3-((1-(3-ethyl-4,7-dimethyl-5-oxo-4,5-dihydro-3H-pyrazolo[3,4-c]isoquinolin-9-yl)ethyl)amino)picolinic acid (I-19)

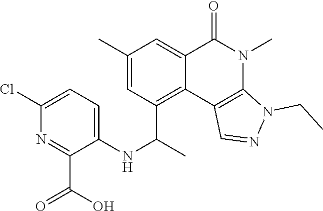 [SMILES: CCn1ncc2c3c(C(C)Nc4ccc(Cl)nc4C(=O)O)cc(C)cc3c(=O)n(C)c21]

The titled compound was prepared using similar procedures as described for Example 16 with 1-ethyl-1H-pyrazol-5-amine replacing 1-methyl-1H-pyrazol-5-amine in Step 1 and 3-amino-6-chloropyridine-2-carboxylic acid replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₂H₂₃ClN₅O₃ (M+H)⁺ m/z=440.2; found 440.2.

### Example 20: 3-((1-(3-Ethyl-4,7-dimethyl-5-oxo-4,5-dihydro-3H-pyrazolo[3,4-c]isoquinolin-9-yl)ethyl)amino)-6-methylpicolinic acid (I-20)

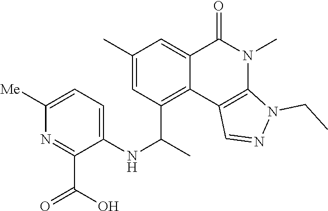 [SMILES: CCn1ncc2c3c(C(C)Nc4ccc(C)nc4C(=O)O)cc(C)cc3c(=O)n(C)c21]

The titled compound was prepared using similar procedures as described for Example 16 with 1-ethyl-1H-pyrazol-5-amine replacing 1-methyl-1H-pyrazol-5-amine in Step 1 and 3-amino-6-methylpicolinic acid replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₆N₅O₃ (M+H)⁺ m/z=420.2; found 420.2.

### Example 21: 2-((1-(4-Ethyl-3,7-dimethyl-5-oxo-4,5-dihydro-3H-pyrazolo[3,4-c]isoquinolin-9-yl)ethyl)amino)benzoic acid (I-21)

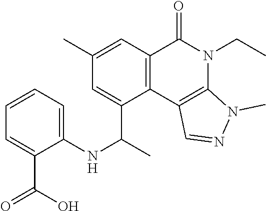 [SMILES: CCn1c(=O)c2cc(C)cc(C(C)Nc3ccccc3C(=O)O)c2c2cnn(C)c21]

The titled compound was prepared using similar procedures as described for Example 16 with iodoethane replacing iodomethane in Step 2. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₅N₄O₃ (M+H)⁺ m/z=405.2; found 405.2.

### Example 22: 6-Chloro-3-((1-(4-ethyl-3,7-dimethyl-5-oxo-4,5-dihydro-3H-pyrazolo[3,4-c]isoquinolin-9-yl)ethyl)amino)picolinic acid (I-22)

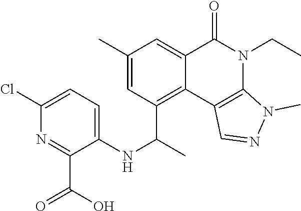 [SMILES: CCn1c(=O)c2cc(C)cc(C(C)Nc3ccc(Cl)nc3C(=O)O)c2c2cnn(C)c21]

The titled compound was prepared using similar procedures as described for Example 16 with iodoethane replacing iodomethane in Step 2 and 3-amino-6-chloropyridine-2-carboxylic acid replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₂H₂₃ClN₅O₃ (M+H)⁺ m/z=440.2; found 440.2.

### Example 23: 2-((1-(3,4-Diethyl-7-methyl-5-oxo-4,5-dihydro-3H-pyrazolo[3,4-c]isoquinolin-9-yl)ethyl)amino)benzoic acid (I-23)

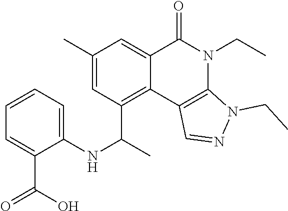 [SMILES: CCn1ncc2c3c(C(C)Nc4ccccc4C(=O)O)cc(C)cc3c(=O)n(CC)c21]

The titled compound was prepared using similar procedures as described for Example 16 with 1-ethyl-1H-pyrazol-5-amine replacing 1-methyl-1H-pyrazol-5-amine in Step 1 and iodoethane replacing iodomethane in Step 2. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₇N₄O₃ (M+H)⁺ m/z=419.2; found 419.2.

### Example 24: 2-((1-(3,4-Diethyl-7-methyl-5-oxo-4,5-dihydro-3H-pyrazolo[3,4-c]isoquinolin-9-yl)ethyl)amino)-5-fluorobenzoic acid (I-24)

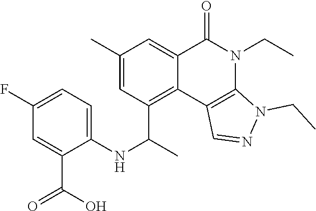 [SMILES: CCn1ncc2c3c(C(C)Nc4ccc(F)cc4C(=O)O)cc(C)cc3c(=O)n(CC)c21]

The titled compound was prepared using similar procedures as described for Example 16 with 1-ethyl-1H-pyrazol-5-amine replacing 1-methyl-1H-pyrazol-5-amine in Step 1, iodoethane replacing iodomethane in Step 2, and 2-amino-5-fluorobenzoic acid replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₆FN₄O₃ (M+H)⁺ m/z=437.2; found 437.2.

### Example 25: 3-((1-(3,4-Diethyl-7-methyl-5-oxo-4,5-dihydro-3H-pyrazolo[3,4-c]isoquinolin-9-yl)ethyl)amino)-6-methylpicolinic acid (I-25)

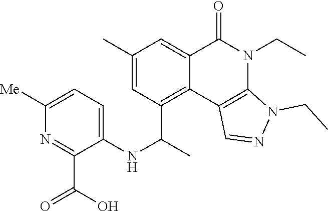 [SMILES: CCn1ncc2c3c(C(C)Nc4ccc(C)nc4C(=O)O)cc(C)cc3c(=O)n(CC)c21]

The titled compound was prepared using similar procedures as described for Example 16 with 1-ethyl-1H-pyrazol-5-amine replacing 1-methyl-1H-pyrazol-5-amine in Step 1, iodoethane replacing iodomethane in Step 2, and 3-amino-6-methylpicolinic acid replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₈N₅O₃ (M+H)⁺ m/z=434.2; found 434.2.

### Example 26: 2-((1-(4,7-Dimethyl-5-oxo-2-propyl-4,5-dihydro-2H-pyrazolo[3,4-c]isoquinolin-9-yl)ethyl)amino)benzoic acid (I-26)

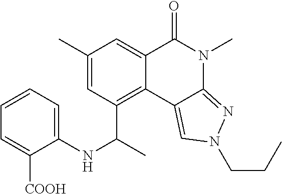 [SMILES: CCCn1cc2c3c(C(C)Nc4ccccc4C(=O)O)cc(C)cc3c(=O)n(C)c2n1]

### Step 1. 3-bromo-2-iodo-N-(1-(4-methoxybenzyl)-1H-pyrazol-5-yl)-5-methylbenzamide

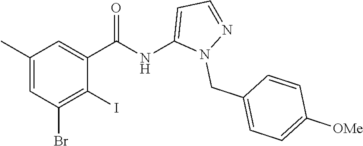 [SMILES: COc1ccc(Cn2nccc2NC(=O)c2cc(C)cc(Br)c2I)cc1]

To a solution of 3-bromo-2-iodo-5-methylbenzoic acid (25.83 g, 75.77 mmol) in DMF (250 mL) was added HATU (37.98 g, 100.0 mmol). The resulting mixture was stirred at room temperature for 15 minutes then 2-[(4-methoxyphenyl)methyl]pyrazol-3-amine (14.0 g, 68.9 mmol) and N,N-diisopropylethylamine (36.0 mL, 206.6 mmol) were added sequentially. The reaction mixture was stirred at room temperature for 24 hours and then poured into water (1500 mL). The resulting solid was collected via filtration, washed with water (500 mL), and dried. The resulting solid was then triturated with ethyl acetate (100 mL) and the suspension was stirred for 4 hours at room temperature. The suspension was filtered to afford the desired product as a yellow solid (22.0 g, 61% yield). LCMS calculated for C₁₉H₁₈BrIN₃O₂ (M+H)⁺ m/z=526.0; found 526.0.

### Step 2. 3-Bromo-2-iodo-N-(1-(4-methoxybenzyl)-1H-pyrazol-5-yl)-N,5-dimethylbenzamide

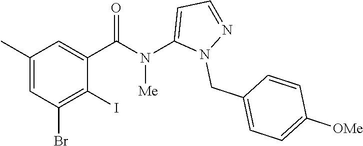 [SMILES: COc1ccc(Cn2nccc2N(C)C(=O)c2cc(C)cc(Br)c2I)cc1]

To a solution of 3-bromo-2-iodo-N-(1-(4-methoxybenzyl)-1H-pyrazol-5-yl)-5-methylbenzamide (9.0 g, 17.10 mmol) in DMF (150 mL) was added NaH (533.6 mg, 22.2 mmol) at 0° C. under nitrogen atmosphere. The resulting mixture was stirred at same temperature for 30 min and then MeI (3.40 g, 23.9 mmol) was added. The reaction mixture was then slowly warmed to rt and stirred for 4 h. After completion, the reaction was carefully quenched with water at 0° C. while vigorously stirred. The mixture was then extracted with ethyl acetate (2×400 mL). The combined organics were dried with anhydrous sodium sulfate, filtered, and concentrated under reduced pressure. The residue was purified by silica gel column chromatography, eluted with hexanes and ethyl acetate to provide the desired product as a white solid (7.6 g, 82% yield). LCMS calculated for C₂₀H₂₀BrIN₃O₂ (M+H)⁺ m/z=540.0; found 540.0.

### Step 3. 9-Bromo-3-(4-methoxybenzyl)-4,7-dimethyl-3,4-dihydro-5H-pyrazolo[3,4-c]isoquinolin-5-one

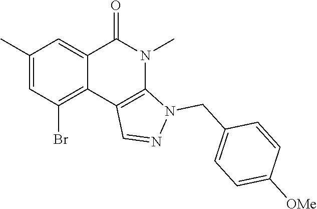 [SMILES: COc1ccc(Cn2ncc3c4c(Br)cc(C)cc4c(=O)n(C)c32)cc1]

A mixture of 3-bromo-2-iodo-N-(1-(4-methoxybenzyl)-1H-pyrazol-5-yl)-N,5-dimethylbenzamide (400 mg, 0.74 mmol), Pd(PPh₃)₄ (427.8 mg, 0.37 mmol) and KOAc (436.0 mg, 4.44 mmol) in DMAc (6.5 mL) was heated at 120° C. for 16 h under nitrogen atmosphere. Upon cooling to room temperature, the mixture was diluted with water and extracted with EtOAc (2×30 mL). The combined organics were washed with sat. NaCl and dried with anhydrous sodium sulfate, filtered, and concentrated under reduced pressure. The residue was purified by silica gel column chromatography, eluted with hexanes and ethyl acetate to provide the desired product (160 mg, 52% yield). LCMS calculated for C₂₀H₁₉BrN₃O₂ (M+H)⁺ m/z=412.1; found 412.1.

### Step 4. 9-Acetyl-3-(4-methoxybenzyl)-4,7-dimethyl-3,4-dihydro-5H-pyrazolo[3,4-c]isoquinolin-5-one

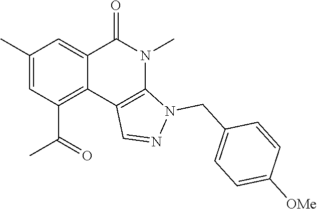 [SMILES: COc1ccc(Cn2ncc3c4c(C(C)=O)cc(C)cc4c(=O)n(C)c32)cc1]

A mixture of 9-bromo-3-(4-methoxybenzyl)-4,7-dimethyl-3,4-dihydro-5H-pyrazolo[3,4-c]isoquinolin-5-one (660 mg, 1.60 mmol), Pd(PPh₃)₄ (370.0 mg, 0.32 mmol), copper iodide (30.5 mg, 0.16 mmol) and tributyl(1-ethoxyvinyl)stannane (1.16 g, 3.20 mmol) in dioxane (16 mL) was heated at 100° C. for 12 h under nitrogen atmosphere. Upon cooling to room temperature, the mixture was added 2 N HCl and stirred for 30 min. Then 400 mg CsF was added and stirred for another 30 min before the mixture was diluted with water and extracted with EtOAc (2×80 mL). The combined organics were washed with sat. NaCl and dried with anhydrous sodium sulfate, filtered, and concentrated under reduced pressure. The residue was purified by silica gel column chromatography, eluted with hexanes and ethyl acetate to provide the desired product (360 mg, 60% yield). LCMS calculated for C₂₂H₂₂N₃O₃ (M+H)⁺ m/z=376.2; found 376.2.

### Step 5. 9-(1-Hydroxyethyl)-3-(4-methoxybenzyl)-4,7-dimethyl-3,4-dihydro-5H-pyrazolo[3,4-c]isoquinolin-5-one

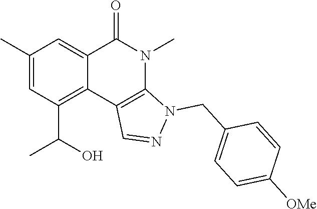 [SMILES: COc1ccc(Cn2ncc3c4c(C(C)O)cc(C)cc4c(=O)n(C)c32)cc1]

To a mixture of 9-acetyl-3-(4-methoxybenzyl)-4,7-dimethyl-3,4-dihydro-5H-pyrazolo[3,4-c]isoquinolin-5-one (360 mg, 1.0 mmol) in THF (10 mL) was added LAH (2.0 mL, 2.0 mmol, 1 M solution in THF) at −78° C. The resulting mixture was stirred at same temperature for 10 min, and then warmed to −40° C. before quenched with sat. NH₄Cl. The mixture was diluted with water and extracted with EtOAc (2×50 mL). The combined organics were washed with sat. NaCl and dried with anhydrous sodium sulfate, filtered, and concentrated under reduced pressure. The residue was purified by silica gel column chromatography, eluted with hexanes and ethyl acetate to provide the desired product (220 mg, 58% yield). LCMS calculated for C₂₂H₂₄N₃O₃ (M+H)⁺ m/z=378.2; found 378.2.

### Step 6. 9-(1-Bromoethyl)-3-(4-methoxybenzyl)-4,7-dimethyl-3,4-dihydro-5H-pyrazolo[3,4-c]isoquinolin-5-one

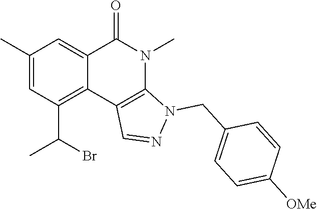 [SMILES: COc1ccc(Cn2ncc3c4c(C(C)Br)cc(C)cc4c(=O)n(C)c32)cc1]

To a mixture of 9-(1-hydroxyethyl)-3-(4-methoxybenzyl)-4,7-dimethyl-3,4-dihydro-5H-pyrazolo[3,4-c]isoquinolin-5-one (200 mg, 0.53 mmol) in DCM (20 mL) was added PBr₃ (215.2 mg, 0.79 mmol). The resulting mixture was stirred at rt for 2 h. The mixture was then quenched with sat. NaHCO₃ and extracted with DCM. The combined organics were washed with sat. NaCl, dried with anhydrous sodium sulfate, filtered, and concentrated under reduced pressure. The residue was used directly in next step without further purification.

### Step 7. Methyl 2-((1-(3-(4-methoxybenzyl)-4,7-dimethyl-5-oxo-4,5-dihydro-3H-pyrazolo[3,4-c]isoquinolin-9-yl)ethyl)amino)benzoate

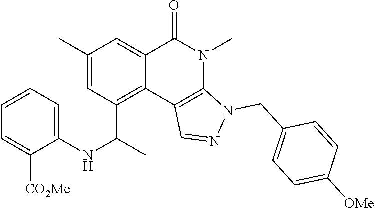 [SMILES: COC(=O)c1ccccc1NC(C)c1cc(C)cc2c(=O)n(C)c3c(cnn3Cc3ccc(OC)cc3)c12]

To a solution of 9-(1-bromoethyl)-3-(4-methoxybenzyl)-4,7-dimethyl-3,4-dihydro-5H-pyrazolo[3,4-c]isoquinolin-5-one (198.2 mg, 0.45 mmol) in DMF (2.0 mL) was added methyl 2-aminobenzoate (204.1 mg, 1.35 mmol) and the resulting reaction mixture was heated at 80° C. for 30 minutes. The mixture was diluted with water and extracted with EtOAc (2×50 mL). The combined organics were washed with sat. NaCl and dried with anhydrous sodium sulfate, filtered, and concentrated under reduced pressure. The residue was used directly in next step without further purification. LCMS calculated for C₃₀H₃₁N₄O₄ (M+H)⁺ m/z=511.2; found 511.2.

### Step 8. Methyl 2-((1-(4,7-dimethyl-5-oxo-4,5-dihydro-3H-pyrazolo[3,4-c]isoquinolin-9-yl)ethyl)amino)benzoate

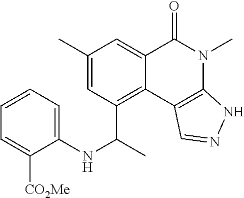 [SMILES: COC(=O)c1ccccc1NC(C)c1cc(C)cc2c(=O)n(C)c3[nH]ncc3c12]

A solution of methyl 2-((1-(3-(4-methoxybenzyl)-4,7-dimethyl-5-oxo-4,5-dihydro-3H-pyrazolo[3,4-c]isoquinolin-9-yl)ethyl)amino)benzoate (230 mg, 0.45 mmol) in TFA (5 ml) and DCM (5 mL) was stirred at 60° C. for 1 hour. The reaction mixture was concentrated under reduced pressure. The resulting solid was dissolved in sat. NaHCO₃ and extracted with ethyl acetate (2×50 mL). The combined organics were washed with sat. NaCl and dried with anhydrous sodium sulfate, filtered, and concentrated under reduced pressure. The residue was purified by silica gel column chromatography, eluted with DCM and ethyl acetate to provide the desired product (140 mg, 80% yield). LCMS calculated for C₂₂H₂₃N₄O₃ (M+H)⁺ m/z=391.2; found 391.1.

### Step 9. Methyl 2-((1-(4,7-dimethyl-5-oxo-2-propyl-4,5-dihydro-2H-pyrazolo[3,4-c]isoquinolin-9-yl)ethyl)amino)benzoate

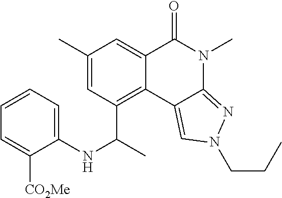 [SMILES: CCCn1cc2c3c(C(C)Nc4ccccc4C(=O)OC)cc(C)cc3c(=O)n(C)c2n1]

To a solution of methyl 2-((1-(4,7-dimethyl-5-oxo-4,5-dihydro-3H-pyrazolo[3,4-c]isoquinolin-9-yl)ethyl)amino)benzoate (20 mg, 0.05 mmol) was added 1-iodopropane (17.4 mg, 0.1 mmol) and potassium carbonate (28.3 mg, 0.2 mmol). The reaction mixture was stirred at 60° C. for 8 hours. The mixture was diluted with water and extracted with EtOAc (2×25 mL). The combined organics were washed with sat. NaCl and dried with anhydrous sodium sulfate, filtered, and concentrated under reduced pressure. The residue was used directly in next step without further purification. LCMS calculated for C₂₅H₂₉N₄O₃ (M+H)⁺ m/z=433.2; found 433.2.

### Step 10. 2-((1-(4,7-Dimethyl-5-oxo-2-propyl-4,5-dihydro-2H-pyrazolo[3,4-c]isoquinolin-9-yl)ethyl)amino)benzoic acid

A solution of methyl 2-((1-(4,7-dimethyl-5-oxo-2-propyl-4,5-dihydro-2H-pyrazolo[3,4-c]isoquinolin-9-yl)ethyl)amino)benzoate (21.6 mg, 0.05 mmol) in THF (0.2 mL), MeOH (0.4 mL) and 2M NaOH aq. Solution (0.6 mL) was stirred at 75° C. for 2 hours. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₇N₄O₃ (M+H)⁺ m/z=419.2; found 419.2.

### Example 27: 2-((1-(4,7-Dimethyl-5-oxo-2-(tetrahydro-2H-pyran-4-yl)-4,5-dihydro-2H-pyrazolo[3,4-c]isoquinolin-9-yl)ethyl)amino)benzoic acid (I-27)

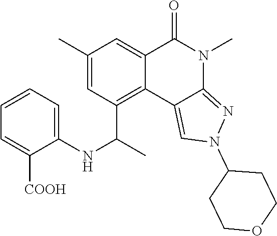 [SMILES: Cc1cc(C(C)Nc2ccccc2C(=O)O)c2c(c1)c(=O)n(C)c1nn(C3CCOCC3)cc21]

The titled compound was prepared using similar procedures as described for Example 26 with 4-bromotetrahydropyran replacing 1-iodopropane in Step 9. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₂₉N₄O₄ (M+H)⁺ m/z=461.2; found 461.2.

### Example 28: 2-((1-(4,7-Dimethyl-5-oxo-2-phenethyl-4,5-dihydro-2/I-pyrazolo[3,4-c]isoquinolin-9-yl)ethyl)amino)benzoic acid (I-28)

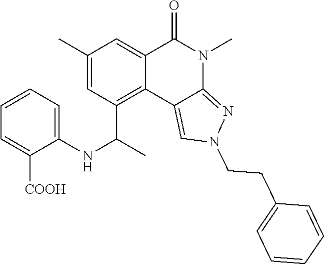 [SMILES: Cc1cc(C(C)Nc2ccccc2C(=O)O)c2c(c1)c(=O)n(C)c1nn(CCc3ccccc3)cc21]

The titled compound was prepared using similar procedures as described for Example 26 with (2-bromoethyl)benzene replacing 1-iodopropane in Step 9. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₂₉N₄O₃ (M+H)⁺ m/z=481.2; found 481.2.

### Example 29: 2-((1-(2-(2-Fluoropyridin-4-yl)-4,7-dimethyl-5-oxo-4,5-dihydro-2H-pyrazolo[3,4-c]isoquinolin-9-yl)ethyl)amino)benzoic acid (I-29)

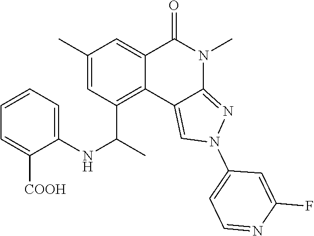 [SMILES: Cc1cc(C(C)Nc2ccccc2C(=O)O)c2c(c1)c(=O)n(C)c1nn(-c3ccnc(F)c3)cc21]

### Step 1. 2-((1-(3-(4-Methoxybenzyl)-4,7-dimethyl-5-oxo-4,5-dihydro-3H-pyrazolo[3,4-c]isoquinolin-9-yl)ethyl)amino)benzoic acid

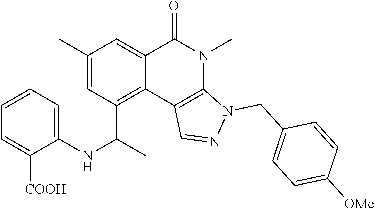 [SMILES: COc1ccc(Cn2ncc3c4c(C(C)Nc5ccccc5C(=O)O)cc(C)cc4c(=O)n(C)c32)cc1]

To a solution of 9-(1-bromoethyl)-3-(4-methoxybenzyl)-4,7-dimethyl-3,4-dihydro-5H-pyrazolo[3,4-c]isoquinolin-5-one (210 mg, 0.48 mmol) in DMF (2.0 mL) was added 2-aminobenzoic acid (197.4 mg, 1.44 mmol) and the resulting reaction mixture was heated at 80° C. for 60 minutes. The mixture was diluted with water and extracted with EtOAc (2×50 mL). The combined organics were washed with sat. NaCl and dried with anhydrous sodium sulfate, filtered, and concentrated under reduced pressure. The residue was used directly in next step without further purification. LCMS calculated for C₂₉H₂₉N₄O₄ (M+H)⁺ m/z=497.2; found 497.2.

### Step 2. 2-((1-(4,7-Dimethyl-5-oxo-4,5-dihydro-3H-pyrazolo[3,4-c]isoquinolin-9-yl)ethyl)amino)benzoic acid

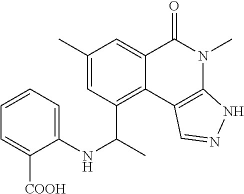 [SMILES: Cc1cc(C(C)Nc2ccccc2C(=O)O)c2c(c1)c(=O)n(C)c1[nH]ncc21]

A solution of 2-((1-(3-(4-methoxybenzyl)-4,7-dimethyl-5-oxo-4,5-dihydro-3H-pyrazolo[3,4-c]isoquinolin-9-yl)ethyl)amino)benzoic acid (238.2 mg, 0.48 mmol) in TFA (5 ml) and DCM (5 mL) was stirred at 60° C. for 1 hour. The reaction mixture was concentrated under reduced pressure. The resulting solid was purified by silica gel column chromatography, eluted with DCM and ethyl acetate to provide the desired product (140 mg, 77% yield). LCMS calculated for C₂₁H₂₁N₄O₃ (M+H)⁺ m/z=377.2; found 377.1.

### Step 3. 2-((1-(2-(2-Fluoropyridin-4-yl)-4,7-dimethyl-5-oxo-4,5-dihydro-2H-pyrazolo[3,4-c]isoquinolin-9-yl)ethyl)amino)benzoic acid

A mixture of 2-((1-(4,7-dimethyl-5-oxo-4,5-dihydro-3H-pyrazolo[3,4-c]isoquinolin-9-yl)ethyl)amino)benzoic acid (15 mg, 0.04 mmol), copper acetate (29.0 mg, 0.16 mmol), pyridine (12.7 mg, 0.16 mmol) and (2-fluoropyridin-4-yl) boronic acid (22.6 mg, 0.16 mmol) in DCM (0.5 mL) was stirred at room temperature for 16 hours. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₂₃FN₅O₃ (M+H)⁺ m/z=472.2; found 472.2.

### Example 30: 2-((1-(4,7-Dimethyl-5-oxo-2-(pyrimidin-5-yl)-4,5-dihydro-2H-pyrazolo[3,4-c]isoquinolin-9-yl)ethyl)amino)benzoic acid (I-30)

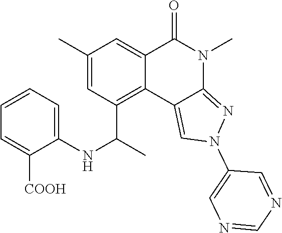 [SMILES: Cc1cc(C(C)Nc2ccccc2C(=O)O)c2c(c1)c(=O)n(C)c1nn(-c3cncnc3)cc21]

The titled compound was prepared using similar procedures as described for Example 29 with pyrimidin-5-ylboronic acid replacing (2-fluoropyridin-4-yl) boronic acid in Step 3. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₂₃N₆O₃ (M+H)⁺ m/z=455.2; found 455.1.

### Example 31: 6-Chloro-3-((1-(4,7-dimethyl-5-oxo-2-(o-tolyl)-4,5-dihydro-2H-pyrazolo[3,4-c]isoquinolin-9-yl)ethyl)amino)picolinic acid (I-31)

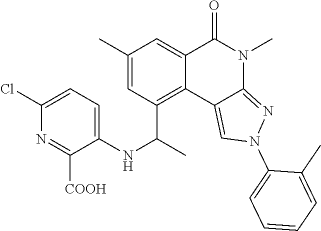 [SMILES: Cc1cc(C(C)Nc2ccc(Cl)nc2C(=O)O)c2c(c1)c(=O)n(C)c1nn(-c3ccccc3C)cc21]

### Step 1. 6-Chloro-3-((1-(3-(4-methoxybenzyl)-4,7-dimethyl-5-oxo-4,5-dihydro-3H-pyrazolo[3,4-c]isoquinolin-9-yl)ethyl)amino)picolinic acid

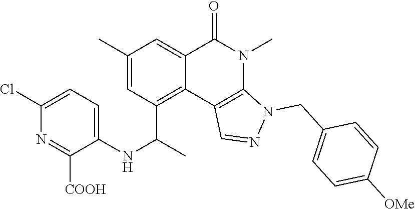 [SMILES: COc1ccc(Cn2ncc3c4c(C(C)Nc5ccc(Cl)nc5C(=O)O)cc(C)cc4c(=O)n(C)c32)cc1]

To a solution of 9-(1-bromoethyl)-3-(4-methoxybenzyl)-4,7-dimethyl-3,4-dihydro-5H-pyrazolo[3,4-c]isoquinolin-5-one (90 mg, 0.20 mmol) in DMF (1 mL) was added 3-amino-6-chloropicolinic acid (103.5 mg, 0.60 mmol) and the resulting reaction mixture was heated at 80° C. for 60 minutes. The mixture was diluted with water and extracted with EtOAc (2×50 mL). The combined organics were washed with sat. NaCl and dried with anhydrous sodium sulfate, filtered, and concentrated under reduced pressure. The residue was used directly in next step without further purification. LCMS calculated for C₂₈H₂₇ClN₅O₄ (M+H)⁺ m/z=532.2; found 532.1.

### Step 2. 6-Chloro-3-((1-(4,7-dimethyl-5-oxo-4,5-dihydro-3H-pyrazolo[3,4-c]isoquinolin-9-yl)ethyl)amino)picolinic acid

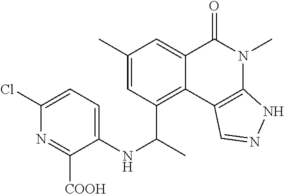 [SMILES: Cc1cc(C(C)Nc2ccc(Cl)nc2C(=O)O)c2c(c1)c(=O)n(C)c1[nH]ncc21]

A solution of 6-chloro-3-((1-(3-(4-methoxybenzyl)-4,7-dimethyl-5-oxo-4,5-dihydro-3H-pyrazolo[3,4-c]isoquinolin-9-yl)ethyl)amino)picolinic acid (106 mg, 0.20 mmol) in TFA (5 ml) and DCM (5 mL) was stirred at 60° C. for 1 hour. The reaction mixture was concentrated under reduced pressure. The resulting solid was purified by silica gel column chromatography, eluted with DCM and ethyl acetate to provide the desired product (49 mg, 60% yield). LCMS calculated for C₂₀H₁₉ClN₅O₃ (M+H)⁺ m/z=412.1; found 412.1.

### Step 3. 6-Chloro-3-((1-(4,7-dimethyl-5-oxo-2-(o-tolyl)-4,5-dihydro-2H-pyrazolo[3,4-c]isoquinolin-9-yl)ethyl)amino)picolinic acid

A mixture of 6-chloro-3-((1-(4,7-dimethyl-5-oxo-4,5-dihydro-3H-pyrazolo[3,4-c]isoquinolin-9-yl)ethyl)amino)picolinic acid (16.5 mg, 0.04 mmol), copper acetate (29.0 mg, 0.16 mmol), pyridine (12.7 mg, 0.16 mmol) and o-tolylboronic acid (21.8 mg, 0.16 mmol) in DCM (0.5 mL) was stirred at room temperature for 16 hours. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₂₅ClN₅O₃ (M+H)⁺ m/z=502.2; found 502.1.

### Example 32: 2-[1-(3-Methoxy-5,8-dimethyl-6-oxo-benzo[c][1,8]naphthyridin-10-yl)ethylamino]benzoic acid (I-32)

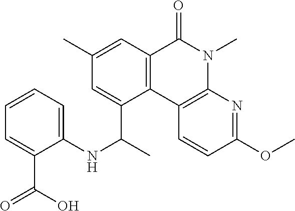 [SMILES: COc1ccc2c3c(C(C)Nc4ccccc4C(=O)O)cc(C)cc3c(=O)n(C)c2n1]

### Step 1: 2-Amino-3-bromo-5-methyl-benzoic acid

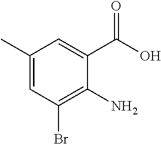

A mixture of 2-amino-5-methyl-benzoic acid, (20.00 g, 132.31 mmol) and 1-bromopyrrolidine-2,5-dione, (25.90 g, 145.54 mmol) in N,N-dimethylformamide (180 mL) was stirred at room temperature for 3 hours. The reaction mixture was poured into water (500 mL) and the solid product was filtered off and washed with water to afford the title compound 2-amino-3-bromo-5-methyl-benzoic acid (29.00 g, 95.3%). LCMS calculated for C₈H₉BrNO₂ (M+H)⁺ m/z=230.0/232.0; found 230.0/231.9.

### Step 2: 3-Bromo-2-iodo-5-methyl-benzoic acid

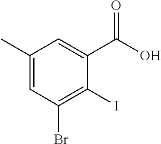

2-Amino-3-bromo-5-methyl-benzoic acid (25 g, 108.6 mmol) was suspended in 120 mL water with stirring. The mixture was cooled in an ice bath and 100 mL concentrated sulfuric acid was added slowly. A solution of sodium nitrite, (13.50 g, 195.60 mmol) in 100 mL water was added dropwise over 0.5 hour. The reaction mixture was stirred for an additional 0.25 hour at 0-5°. The mixture was poured into a well-stirred solution of potassium iodide (27.06 g, 163.00 mmol) in 100 mL water. The resulting solid was collected by filtration and stirred in 200 mL ethyl acetate. A solution of 200 mL 20% sodium hydrogen sulfite was added slowly. The organic layer was separated, washed with 100 mL 20% sodium hydrogen sulfite, 50 mL saturated sodium chloride, dried (magnesium sulfate) and concentrated under reduced pressure. The residue was recrystallized from ether/hexane to provide 3-bromo-2-iodo-5-methyl-benzoic acid (22.00 g, 59.4%).

### Step 3: 3-Bromo-2-iodo-N,5-dimethyl-benzamide

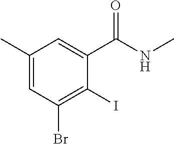

To a stirred solution of 3-bromo-2-iodo-5-methyl-benzoic acid (20.00 g, 58.66 mmol) in THF (250 mL) was added [dimethylamino (triazolo[4,5-b]pyridin-3-yloxy)methylene]-dimethyl-ammonium hexafluorophosphate (44.61 g, 117.32 mmol) and N-ethyl-N-isopropyl-propan-2-amine (18.39 mL, 105.59 mmol) and the resulting mixture was kept stirred for 15 min. methanamine (3.64 g, 117.32 mmol) was then added to the reaction mixture and the resulting mixture was kept stirred at rt overnight. LCMS analysis indicated the reaction was complete. The reaction was quenched with water (200 mL) and THF was partially removed. The suspension was stirred for 30 min until precipitate formed. The precipitate was collected by filtration and washed with water to afford 3-bromo-2-iodo-N,5-dimethyl-benzamide (18.80 g, 90.5%). LCMS calculated for C₉H₁₀BrINO (M+H)⁺ m/z=353.9/355.9; found 354.0/356.0.

### Step 4: 10-Bromo-3-fluoro-5,8-dimethyl-benzo[c][1,8]naphthyridin-6-one

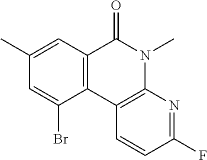 [SMILES: Cc1cc(Br)c2c(c1)c(=O)n(C)c1nc(F)ccc21]

A solution of 3-bromo-2-iodo-N,5-dimethyl-benzamide (10.00 g, 28.25 mmol), (2,6-difluoro-3-pyridyl) boronic acid (8.08 g, 50.85 mmol), dicesium carbonate (23.01 g, 70.62 mmol) and palladium triphenylphosphine (7.83 g, 6.78 mmol) in 100 mL toluene, 50 mL EtOH and 50 mL H₂O was bubbled with N₂ for 5 min. And the resulting reaction mixture was stirred at 70° C. overnight. LCMS analysis indicated the reaction was complete. The mixture was then extracted with ethyl acetate (2×150 mL). The combined organics were dried with anhydrous sodium sulfate, filtered, and concentrated under reduced pressure. The residue was purified by silica gel column chromatography, eluted with hexanes, DCM and ethyl acetate to provide the desired product 10-bromo-3-fluoro-5,8-dimethyl-benzo[c][1,8]naphthyridin-6-one (2.85 g, 31.4%). LCMS calculated for C₁₄H₁₁BrFN₂O (M+H)⁺ m/z=321.0/323.0; found 321.0/323.0.

### Step 5: 3-Fluoro-5,8-dimethyl-10-vinyl-benzo[c][1,8]naphthyridin-6-one

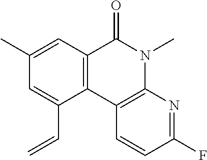 [SMILES: C=Cc1cc(C)cc2c(=O)n(C)c3nc(F)ccc3c12]

A mixture of 10-bromo-3-fluoro-5,8-dimethyl-benzo[c][1,8]naphthyridin-6-one (2.50 g, 7.78 mmol), potassium trifluoro (vinyl) borate (3.13 g, 23.35 mmol), dipotassium carbonate (3.23 g, 23.35 mmol) and palladium triphenylphosphine (1.35 g, 1.17 mmol) in THF (40 mL) and water (10 mL) was stirred at 70° C. for 12 h under nitrogen atmosphere. Upon cooling to room temperature, the mixture was diluted with water (100 mL) with stirring. The white precipitate was collected by filtration and washed with water. The crude product was directly used in the following step without further purification. LCMS calculated for C₁₆H₁₄FN₂O (M+H)⁺ m/z=269.1; found 269.1.

### Step 6: 3-Fluoro-5,8-dimethyl-6-oxo-benzo[c][1,8]naphthyridine-10-carbaldehyde

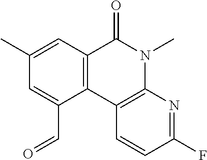 [SMILES: Cc1cc(C=O)c2c(c1)c(=O)n(C)c1nc(F)ccc21]

To a stirred solution of 3-fluoro-5,8-dimethyl-10-vinyl-benzo[c][1,8]naphthyridin-6-one (2.10 g, 7.83 mmol), sodium periodate (8.37 g, 39.14 mmol) and 2,6-dimethylpyridine (1.81 mL, 15.65 mmol) in 50 mL THF and 15 mL H₂O was added tetraoxoosmium (0.16 g, 0.63 mmol) (4% aq. solution) dropwise and the resulting reaction mixture was kept stirred at rt for 3 h. LCMS analysis indicated the reaction was complete. The reaction mixture was diluted with water and extracted with EtOAc. The combined organics were washed with sat. NaCl and dried with anhydrous sodium sulfate, filtered, and concentrated under reduced pressure to afford 3-fluoro-5,8-dimethyl-6-oxo-benzo[c][1,8]naphthyridine-10-carbaldehyde (1.81 g, 85%) as a crude product which was directly used in the following step without further purification. LCMS calculated for C₁₅H₁₂FN₂O₂ (M+H)⁺ m/z=271.1; found 271.1.

### Step 7: 3-Fluoro-10-(1-hydroxyethyl)-5,8-dimethyl-benzo[c][1,8]naphthyridin-6-one

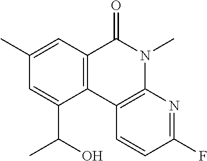 [SMILES: Cc1cc(C(C)O)c2c(c1)c(=O)n(C)c1nc(F)ccc21]

To a stirred solution of 3-fluoro-5,8-dimethyl-6-oxo-benzo[c][1,8]naphthyridine-10-carbaldehyde (1.80 g, 6.66 mmol) in 50 mL THF was added bromo(methyl)magnesium (6.66 mL, 19.98 mmol) dropwise at −78° C. The resulting mixture was stirred at same temperature for 10 min, and then warmed to rt. LCMS analysis indicated the reaction was complete. The reaction was quenched with sat. NH₄Cl and diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product 3-fluoro-10-(1-hydroxyethyl)-5,8-dimethyl-benzo[c][1,8]naphthyridin-6-one (710 mg, 37%). LCMS calculated for C₁₆H₁₆FN₂O₂ (M+H)⁺ m/z=287.1; found 287.1.

### Step 8: 1-(3-Fluoro-5,8-dimethyl-6-oxo-benzo[c][1,8]naphthyridin-10-yl)ethyl methanesulfonate

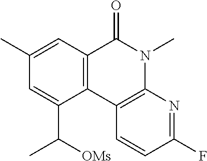 [SMILES: Cc1cc(C(C)OS(C)(=O)=O)c2c(c1)c(=O)n(C)c1nc(F)ccc21]

To a stirred solution of 3-fluoro-10-(1-hydroxyethyl)-5,8-dimethyl-benzo[c][1,8]naphthyridin-6-one (70 mg, 0.24 mmol) and methanesulfonyl chloride (28 uL, 0.37 mmol) in 2 mL DCM was added N,N-diethylethanamine (136 uL, 0.98 mmol) at 0° C. The resulting mixture was stirred at same temperature for 10 min, and then warmed to rt and stirred for 1 h. The reaction mixture was concentrated to dryness and the crude was directly used in the following step without further purification.

### Step 9: 2-[1-(3-Fluoro-5,8-dimethyl-6-oxo-benzo[c][1,8]naphthyridin-10-yl)ethylamino]benzoic acid

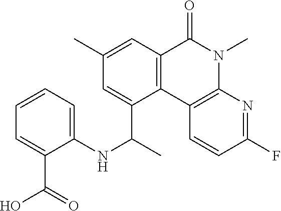 [SMILES: Cc1cc(C(C)Nc2ccccc2C(=O)O)c2c(c1)c(=O)n(C)c1nc(F)ccc21]

1-(3-Fluoro-5,8-dimethyl-6-oxo-benzo[c][1,8]naphthyridin-10-yl)ethyl methanesulfonate (80 mg, 0.22 mmol) in 2 dram vial was added DMF (1 mL) and 2-aminobenzoic acid (90 mg, 0.66 mmol). The reaction mixture was stirred at 100° C. for 12 h. Upon cooling to room temperature, the mixture was diluted with water and extracted with EtOAc 3 times. The combined organics were washed with sat. NaCl, dried with anhydrous sodium sulfate, filtered, and concentrated under reduced pressure. The residue was purified by silica gel column chromatography, eluted with DCM and ethyl acetate to provide the desired product (36 mg). LCMS calculated for C₂₃H₂₁FN₃O₃ (M+H)⁺ m/z=406.2; found 406.2.

### Step 10: 2-[1-(3-Methoxy-5,8-dimethyl-6-oxo-benzo[c][1,8]naphthyridin-10-yl)ethylamino]benzoic acid

2-[1-(3-Fluoro-5,8-dimethyl-6-oxo-benzo[c][1,8]naphthyridin-10-yl)ethylamino]benzoic acid, (8 mg, 0.02 mmol) in 2 dram vial was added DMF (0.3 mL) and sodium methanolate (3.2 mg, 0.06 mmol). The mixture was heated to 90° C. for 2 h. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product. LCMS calculated for C₂₄H₂₃N₃Na₀₄ (M+Na)⁺ m/z=440.2; found 440.2.

### Example 33: 2-[1-[3-(Dimethylamino)-5,8-dimethyl-6-oxo-benzo[c][1,8]naphthyridin-10-yl]ethylamino]benzoic acid (I-33)

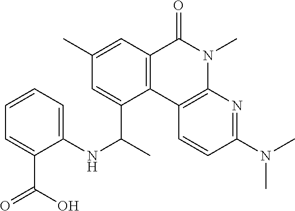 [SMILES: Cc1cc(C(C)Nc2ccccc2C(=O)O)c2c(c1)c(=O)n(C)c1nc(N(C)C)ccc21]

2-[1-(3-Fluoro-5,8-dimethyl-6-oxo-benzo[c][1,8]naphthyridin-10-yl)ethylamino]benzoic acid, (6.0 mg, 0.01 mmol) in 2 dram vial was added DMF (0.3 mL) and N-methylmethanamine (29.6 uL, 0.06 mmol). The mixture was heated to 90° C. for 2 h. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product. LCMS calculated for C₂₅H₂₆N₄NaO₃ (M+Na)⁺ m/z=453.2; found 453.2.

### Example 34: 2-[1-[3-(2-Methoxyethoxy)-5,8-dimethyl-6-oxo-benzo[c][1,8]naphthyridin-10-yl]ethylamino]benzoic acid (I-34)

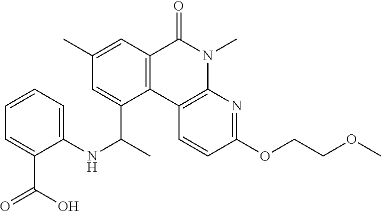 [SMILES: COCCOc1ccc2c3c(C(C)Nc4ccccc4C(=O)O)cc(C)cc3c(=O)n(C)c2n1]

To a stirred solution of 2-methoxyethanol (100 mg, 1.31 mmol) in 1 mL THF was added sodium hydride (60% dispersion in mineral oil, 47.3 mg, 1.18 mmol) at 0° C. The reaction mixture was stirred at 0° C. for 1 h. And 50 μL of the above solution was added to a solution of 2-[1-(3-fluoro-5,8-dimethyl-6-oxo-benzo[c][1,8]naphthyridin-10-yl)ethylamino]benzoic acid (6.0 mg, 0.01 mmol) in DMF (0.3 mL). The mixture was heated to 90° C. for 2 h. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product. LCMS calculated for C₂₆H₂₇N₃NaO₅ (M+Na)⁺ m/z=484.2; found 484.2.

### Example 35: 2-((1-(7-Chloro-3,4-dimethyl-5-oxo-4,5-dihydro-3H-pyrazolo[3,4-c]isoquinolin-9-yl)ethyl)amino)benzoic acid (I-35)

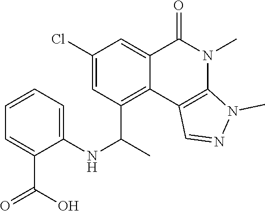 [SMILES: CC(Nc1ccccc1C(=O)O)c1cc(Cl)cc2c(=O)n(C)c3c(cnn3C)c12]

The titled compound was prepared using similar procedures as described for Example 16 with 3-3-bromo-5-chloro-2-iodobenzoic acid replacing 3-bromo-2-iodo-5-methylbenzoic acid in Step 1. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₁H₂₀ClN₄O₃ (M+H)⁺ m/z=411.1; found 411.1.

### Example 36: 2-((1-(8-Methyl-6-oxo-4,5-dihydro-3H,6H-2,2a,5a-triazaaceanthrylen-10-yl)ethyl)amino)benzoic acid (I-36)

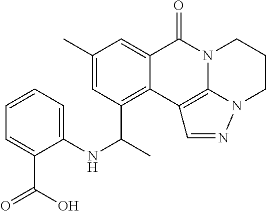 [SMILES: Cc1cc(C(C)Nc2ccccc2C(=O)O)c2c(c1)c(=O)n1c3c2cnn3CCC1]

### Step 1: 3-Bromo-2-iodo-5-methylbenzoic acid

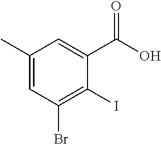

2-Amino-3-bromo-5-methyl-benzoic acid (25 g, 108.6 mmol) was suspended in 120 mL water with stirring. The mixture was cooled in an ice bath and 100 mL concentrated sulfuric acid was added slowly. A solution of sodium nitrite (13.50 g, 195.60 mmol) in 100 mL water was added dropwise over 0.5 hour. The reaction mixture was stirred for an additional 0.25 hour at 0-5° C. The mixture was poured into a well-stirred solution of potassium iodide (27.06 g, 163.00 mmol) in 100 mL water. The resulting solid was collected by filtration and stirred in 200 mL ethyl acetate. A solution of 200 mL 20% sodium hydrogen sulfite was added slowly. The organic layer was separated, washed with 100 mL 20% sodium hydrogen sulfite, 50 mL saturated sodium chloride, dried (magnesium sulfate) and concentrated under reduced pressure. The residue was recrystallized from ether/hexane to provide 3-bromo-2-iodo-5-methyl-benzoic acid (22.00 g, 59.4%). LCMS calculated for C₈H₇BrIO₂ (M+H)⁺ m/z=340.9; found 340.9.

### Step 2: (3-Bromo-2-iodo-5-methylphenyl) (6,7-dihydropyrazolo[1,5-a]pyrimidin-4(5H)-yl)methanone

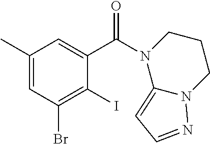 [SMILES: Cc1cc(Br)c(I)c(C(=O)N2CCCn3nccc32)c1]

To a mixture of 3-bromo-2-iodo-5-methylbenzoic acid (664 mg, 1.95 mmol) in DMF (5 mL) was added HATU (741 mg, 1.95 mmol), 4,5,6,7-tetrahydropyrazolo[1,5-a]pyrimidine (200 mg, 1.62 mmol) and DIPEA (0.85 mL, 4.87 mmol). The reaction mixture was stirred at r.t. (room temperature) for 4 h. After completion, the reaction was quenched with water. The mixture was then extracted with ethyl acetate (2×300 mL). The combined organics were dried with anhydrous sodium sulfate, filtered, and concentrated under reduced pressure, which was further purified by silica gel column chromatography, eluted with DCM and ethyl acetate to provide the desired product as white solid (520 mg, 72%). LCMS calculated for C₁₄H₁₄BrIN₃O (M+H)⁺ m/z=445.9; found 445.9.

### Step 3: 10-Bromo-8-methyl-4,5-dihydro-3H,6H-2,2a,5a-triazaaceanthrylen-6-one

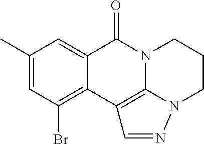 [SMILES: Cc1cc(Br)c2c(c1)c(=O)n1c3c2cnn3CCC1]

A mixture of (3-bromo-2-iodo-5-methylphenyl) (6,7-dihydropyrazolo[1,5-a]pyrimidin-4(5H)-yl)methanone (500 mg, 1.12 mmol), Pd(PPh₃)₄ (647 mg, 0.56 mmol), KOAc (660 mg, 6.73 mmol) in DMAc (5 mL) was heated at 120° C. for 16 h under nitrogen atmosphere. Upon cooling to room temperature, the mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a white solid (210 mg, 59%). LCMS calculated for C₁₄H₁₃BrN₃O (M+H)⁺ m/z=318.0; found 318.0.

### Step 4: 10-Acetyl-8-methyl-4,5-dihydro-3H,6H-2,2a,5a-triazaaceanthrylen-6-one

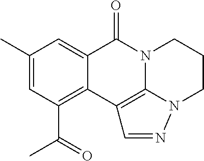 [SMILES: CC(=O)c1cc(C)cc2c(=O)n3c4c(cnn4CCC3)c12]

A mixture of 10-bromo-8-methyl-4,5-dihydro-3H,6H-2,2a,5a-triazaaceanthrylen-6-one (210 mg, 0.66 mmol), PdCl₂(PPh₃)₂ (92 mg, 0.13 mmol), and tributyl(1-ethoxyvinyl)stannane (357 mg, 0.99 mmol) in dioxane (5 mL) was heated at 100° C. for 2 h under nitrogen atmosphere. Upon cooling to room temperature, the mixture was added 2 N HCl and stirred for 30 min. Then 200 mg CsF was added and stirred for another 30 min before the mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a white solid (150 mg, 81%). LCMS calculated for C₁₆H₁₆N₃O₂ (M+H)⁺ m/z=282.1; found 282.1.

### Step 5: 10-(1-Hydroxyethyl)-8-methyl-4,5-dihydro-3H,6H-2,2a,5a-triazaaceanthrylen-6-one

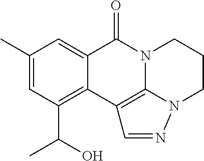 [SMILES: Cc1cc(C(C)O)c2c(c1)c(=O)n1c3c2cnn3CCC1]

To a mixture of 10-acetyl-8-methyl-4,5-dihydro-3H,6H-2,2a,5a-triazaaceanthrylen-6-one (150 mg, 0.53 mmol) in THF (2 mL) was added LAH (0.8 mL, 0.8 mmol, 1 M solution in THF) at −78° C. The resulting mixture was stirred at same temperature for 10 min, and then warmed to −40° C. before quenched with sat. NH₄Cl. The mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a white solid (140 mg, 92%). LCMS calculated for C₁₆H₁₈N₃O₂ (M+H)⁺ m/z=284.1; found 284.1.

### Step 6: 10-(1-Bromoethyl)-8-methyl-4,5-dihydro-3H,6H-2,2a,5a-triazaaceanthrylen-6-one

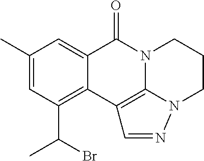 [SMILES: Cc1cc(C(C)Br)c2c(c1)c(=O)n1c3c2cnn3CCC1]

To a mixture of 10-(1-hydroxyethyl)-8-methyl-4,5-dihydro-3H,6H-2,2a,5a-triazaaceanthrylen-6-one (20 mg, 0.07 mmol) in DCM (1 mL) was added PBr₃ (38 mg, 0.14 mmol). The resulting mixture was stirred at rt for 2 h. The mixture was then quenched with sat. NaHCO₃ and extracted with DCM. The combined organics were washed with sat. NaCl, dried with anhydrous sodium sulfate, filtered, and concentrated under reduced pressure. The residue was used directly in next step without further purification.

### Step 7: 2-((1-(8-Methyl-6-oxo-4,5-dihydro-3H,6H-2,2a,5a-triazaaceanthrylen-10-yl)ethyl)amino)benzoic acid

To 10-(1-Bromoethyl)-8-methyl-4,5-dihydro-3H,6H-2,2a,5a-triazaaceanthrylen-6-one (20 mg, 0.06 mmol) in 2 dram vial was added DMF (0.3 mL) and 2-aminobenzoic acid (20 mg, 0.14 mmol). The mixture was heated to 90° C. for 2 h. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₃N₄O₃ (M+H)⁺ m/z=403.2; found 403.2.

### Example 37: 6-Methyl-3-((1-(8-methyl-6-oxo-4,5-dihydro-3H,6H-2,2a,5a-triazaaceanthrylen-10-yl)ethyl)amino)picolinic acid (1-37)

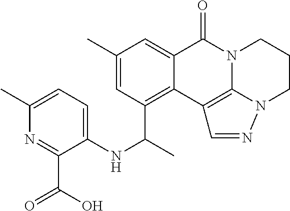 [SMILES: Cc1cc(C(C)Nc2ccc(C)nc2C(=O)O)c2c(c1)c(=O)n1c3c2cnn3CCC1]

The titled compound was prepared using similar procedures as described for Example 36 with 3-amino-6-methylpicolinic acid replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₄N₅O₃ (M+H)⁺ m/z=418.2; found 418.2.

### Example 38: 2-((1-(3,8-Dimethyl-6-oxo-4,5-dihydro-3H,6H-2,2a,5a-triazaaceanthrylen-10-yl)ethyl)amino)benzoic acid (I-38)

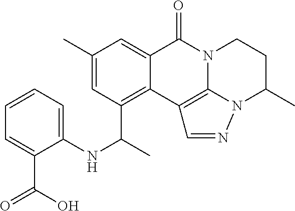 [SMILES: Cc1cc(C(C)Nc2ccccc2C(=O)O)c2c(c1)c(=O)n1c3c2cnn3C(C)CC1]

The titled compound was prepared using similar procedures as described for Example 36 with 7-methyl-4,5,6,7-tetrahydropyrazolo[1,5-a]pyrimidine replacing 4,5,6,7-tetrahydropyrazolo[1,5-a]pyrimidine in Step 2. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₅N₄O₃ (M+H)⁺ m/z=417.2; found 417.2.

### Example 39: 6-Chloro-3-((1-(3,8-dimethyl-6-oxo-4,5-dihydro-3H,6H-2,2a,5a-triazaaceanthrylen-10-yl)ethyl)amino)picolinic acid (I-39)

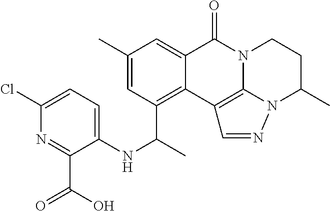 [SMILES: Cc1cc(C(C)Nc2ccc(Cl)nc2C(=O)O)c2c(c1)c(=O)n1c3c2cnn3C(C)CC1]

The titled compound was prepared using similar procedures as described for Example 36 with 7-methyl-4,5,6,7-tetrahydropyrazolo[1,5-a]pyrimidine replacing 4,5,6,7-tetrahydropyrazolo[1,5-a]pyrimidine in Step 2, and 3-amino-6-chloropicolinic acid replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₃ClN₅O₃ (M+H)⁺ m/z=452.1; found 452.1.

### Example 40. 2-Ethyl-3,6-dimethyl-8-((6-methyl-[2,4′-bipyridin]-3-yl)amino)-8,9-dihydrobenzo[de]pyrazolo[4,5,1-ij][1,7]naphthyridin-4(3H)-one

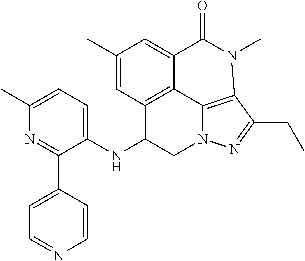 [SMILES: CCc1nn2c3c4c(cc(C)cc4c(=O)n(C)c13)C(Nc1ccc(C)nc1-c1ccncc1)C2]

### Step 1. 2-Bromo-3-iodo-5-methylbenzoic acid

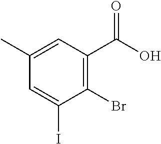

In a 1 L three necked flask fitted with a dropping funnel and thermometer, were charged 2-amino-3-iodo-5-methylbenzoic acid (17.8 g, 64.2 mmol) and acetonitrile (160 mL). After cooling to 0° C., HBr (47%, 160 mL) was added drop wise over 10 min. To the resulting solution a solution of NaNO₂ (4.88 g, 70.7 mmol) in water (20.0 mL) was added drop wise over 1 h. After addition, the reaction mixture was stirred at 0° C. for 30 min, and copper (I) bromide (11.06 g, 77.1 mmol) was added portion wise over 30 min. The resulting mixture was then heated at 70° C. in an oil bath for 1 h. After cooling to 0° C., 700 mL of water was added and the precipitate was filtered, washed with cold water and dried under vacuum to give the title compound as a white solid. The crude product was used in the next step without further purification.

### Step 2. 2-Bromo-3-iodo-N,N,5-trimethylbenzamide

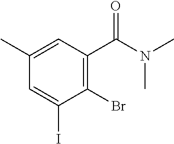

To a stirred solution of 2-bromo-3-iodo-5-methylbenzoic acid (5.0 g, 14.7 mmol) in DMF (50 mL) was added HATU (7.2 g, 19.1 mmol), dimethylamine (2M THF, 8 mL, 16 mmol) and N-ethyl-N-isopropyl-propan-2-amine (3.33 mL, 19.1 mmol). The resulting mixture was kept stirring overnight before quenched with ice water. The mixture was extracted with EtOAc. The combined organics were washed with sat. NaCl, dried with anhydrous sodium sulfate, filtered, and concentrated under reduced pressure. The residue was purified by silica gel column chromatography, eluted with hexanes and ethyl acetate to provide the desired product as a brown solid (4.8 g, 90%). LCMS calculated for C₁₀H₁₂BrINO (M+H)⁺ m/z=368.0; found 368.0.

### Step 3. 2-Bromo-N,N,5-trimethyl-3-vinylbenzamide

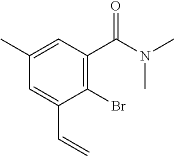

The mixture of 2-bromo-3-iodo-N,N,5-trimethylbenzamide (3.0 g, 8.8 mmol), PdCl₂(PPh₃)₂ (572 mg, 0.82 mmol), and tributyl(vinyl)stannane (2.84 g, 8.97 mmol) in dioxane (50 mL) was heated at 100° C. for 2 h under nitrogen atmosphere. Upon cooling to room temperature, the mixture was diluted with water and extracted with EtOAc. The combined organics were washed with sat. NaCl, dried with anhydrous sodium sulfate, filtered, and concentrated under reduced pressure. The residue was purified by silica gel column chromatography, eluted with DCM and ethyl acetate to provide the desired product as a brown solid (2.0 g, 85%). LCMS calculated for C₁₂H₁₅BrNO (M+H)⁺ m/z=268.0; found 268.1.

### Step 4. 2-Bromo-N,N,5-trimethyl-3-(oxiran-2-yl)benzamide

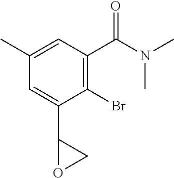

To a mixture of 2-bromo-N,N,5-trimethyl-3-vinylbenzamide (4.3 g, 16.1 mmol) in DCM (500 mL) was added mCPBA (4.7 g, 19.2 mmol) at 0° C. in three portions over 1 h. The reaction mixture was then warmed to rt and stirred for 2 h. After completion, the reaction was diluted with ice water and washed with sat NaHCO₃ and sat. NaCl, dried with anhydrous sodium sulfate, filtered, and concentrated under reduced pressure to give the title compound as a light-yellow oil. The crude product was used in the next step without further purification.

### Step 5. 2-Bromo-3-(1-hydroxy-2-(4-nitro-1H-pyrazol-1-yl)ethyl)-N,N,5-trimethylbenzamide

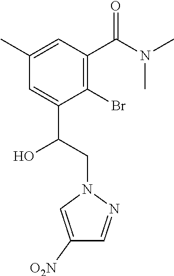

To a mixture of 2-bromo-N,N,5-trimethyl-3-(oxiran-2-yl)benzamide (2.0 g, 7.1 mmol) in PhCF₃ (100 mL) was added 4-nitro-1H-pyrazole (1.6 g, 14.1 mmol) and Cs₂CO₃ (1.1 g, 3.5 mmol) at rt. The resulting mixture was stirred at 110° C. for 6 h. Upon cooling to room temperature, the reaction was diluted with water and extracted with DCM/IPA 3:1. The combined organics were washed with sat. NaCl, dried with anhydrous sodium sulfate, filtered, and concentrated under reduced pressure. The residue was purified by silica gel column chromatography, eluted with DCM and MeOH to provide the desired product as a brown oil (1.3 g, 45%). LCMS calculated for C₁₅H₁₈BrN₄O₄ (M+H)⁺ m/z=397.0; found 397.1.

### Step 6. 2-Bromo-N,N,5-trimethyl-3-(2-(4-nitro-1H-pyrazol-1-yl)-1-((2-(trimethylsilyl)ethoxy)methoxy)ethyl)benzamide

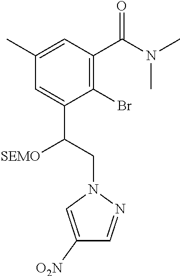

To a mixture of 2-bromo-3-(1-hydroxy-2-(4-nitro-1H-pyrazol-1-yl)ethyl)-N,N,5-trimethylbenzamide (830 mg, 2.1 mmol) in DMF (8 mL) was added NaH (100 mg, 2.5 mmol) at 0° C. under nitrogen atmosphere. The resulting mixture was stirred at same temperature for 30 min and then SEMCl (418 mg, 2.5 mmol) was added. The reaction mixture was then slowly warmed to rt and stirred for 1 h. After completion, the reaction was carefully quenched with water at 0° C. while vigorously stirred. The mixture was then extracted with ethyl acetate (2×50 mL). The combined organics were dried with anhydrous sodium sulfate, filtered, and concentrated under reduced pressure. The residue was purified by silica gel column chromatography, eluted with hexanes and ethyl acetate to provide the desired product, which was further purified by silica gel column chromatography, eluted with hexanes and ethyl acetate to provide the desired product as a white solid (883 mg, 80%). LCMS calculated for C₂₁H₃₂BrN₄O₅Si (M+H)⁺ m/z=527.1; found 527.1.

### Step 7. N,N,8-Trimethyl-1-nitro-6-((2-(trimethylsilyl) ethoxy) methoxy)-5,6-dihydropyrazolo[5,1-a]isoquinoline-10-carboxamide

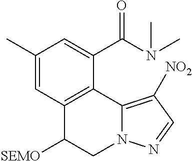

The mixture of 2-bromo-N,N,5-trimethyl-3-(2-(4-nitro-1H-pyrazol-1-yl)-1-((2-(trimethylsilyl)ethoxy)methoxy)ethyl)benzamide (883 mg, 1.68 mmol), Pd(PPh₃)₄ (576 mg, 0.5 mmol), KOAc (1.0 g, 10.1 mmol) in DMAc (10 mL) was heated at 120° C. for 2 h under nitrogen atmosphere. Upon cooling to room temperature, the mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with hexanes and ethyl acetate to provide the desired product as a white solid (446 mg, 65%). LCMS calculated for C₂₁H₃₁N₄O₅Si (M+H)⁺ m/z=447.2; found 447.2.

### Step 8: 1-Amino-N,N,8-trimethyl-6-((2-(trimethylsilyl) ethoxy) methoxy)-5,6-dihydropyrazolo[5,1-a]isoquinoline-10-carboxamide

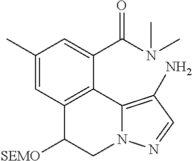

The mixture of N,N,8-trimethyl-1-nitro-6-((2-(trimethylsilyl) ethoxy) methoxy)-5,6-dihydropyrazolo[5,1-a]isoquinoline-10-carboxamide (446 mg, 1.0 mmol), and Fe (223 mg, 4 mmol), in MeOH (3 mL), THF (3 mL) and sat. NH₄Cl (1.5 mL) was heated at 60° C. for 2 h under nitrogen atmosphere. Upon cooling to room temperature, the mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and MeOH to provide the desired product as a brown oil (183 mg, 44%). LCMS calculated for C₂₁H₃₃N₄O₃Si (M+H)⁺ m/z=417.2; found 417.2.

### Step 9: 6-Methyl-8-((2-(trimethylsilyl) ethoxy) methoxy)-8,9-dihydrobenzo[de]pyrazolo[4,5,1-ij][1,7]naphthyridin-4(3H)-one

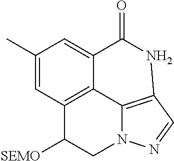

The mixture of 1-amino-N,N,8-trimethyl-6-((2-(trimethylsilyl) ethoxy) methoxy)-5,6-dihydropyrazolo[5,1-a]isoquinoline-10-carboxamide (183 mg, 0.44 mmol) in THF (3 mL) was added LDA (2M in THF, 0.3 mL, 0.6 mmol) at −78° C. under nitrogen atmosphere. After stirring for 10 min at −78° C., the mixture was diluted sat. NH₄Cl and extracted with EtOAc. The combined organics were washed with sat. NaCl and dried with anhydrous sodium sulfate, filtered, and concentrated under reduced pressure. The residue was purified by silica gel column chromatography, eluted with DCM and MeOH to provide the desired product as a brown oil (127 mg, 78%). LCMS calculated for C₁₉H₂₆N₃O₃Si (M+H)⁺ m/z=372.2; found 372.2.

### Step 10: 3,6-Dimethyl-8-((2-(trimethylsilyl) ethoxy) methoxy)-8,9-dihydrobenzo[de]pyrazolo[4,5,1-ij][1,7]naphthyridin-4(3H)-one

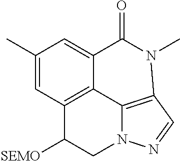

To a mixture of 6-methyl-8-((2-(trimethylsilyl) ethoxy) methoxy)-8,9-dihydrobenzo[de]pyrazolo[4,5,1-ij][1,7]naphthyridin-4(3H)-one (127 mg, 0.34 mmol) in DMF (2 mL) was added NaH (17 mg, 0.44 mmol) at 0° C. under nitrogen atmosphere. The resulting mixture was stirred at same temperature for 30 min and then MeI (62 mg, 0.44 mmol) was added. The reaction mixture was then slowly warmed to rt and stirred for 1 h. After completion, the reaction was carefully quenched with water at 0° C. while vigorously stirred. The mixture was then extracted with ethyl acetate (2×5 mL). The combined organics were dried with anhydrous sodium sulfate, filtered, and concentrated under reduced pressure. The residue was purified by silica gel column chromatography, eluted with hexanes and ethyl acetate to provide the desired product, which was further purified by silica gel column chromatography, eluted with DCM and MeOH to provide the desired product as a white solid (102 mg, 80%). LCMS calculated for C₂₀H₂₈N₃O₃Si (M+H)⁺ m/z=386.2; found 386.2.

### Step 11. 2-Bromo-3,6-dimethyl-8-((2-(trimethylsilyl) ethoxy) methoxy)-8,9-dihydrobenzo[de]pyrazolo[4,5,1-ij][1,7]naphthyridin-4(3H)-one

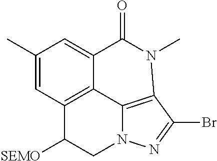

To a mixture of 2-bromo-3,6-dimethyl-8-((2-(trimethylsilyl) ethoxy) methoxy)-8,9-dihydrobenzo[de]pyrazolo[4,5,1-ij][1,7]naphthyridin-4(3H)-one (102 mg, 0.22 mmol) in DCM (2 mL) was added NBS (39 mg, 0.22 mmol) at 0° C. under nitrogen atmosphere. The reaction mixture was then slowly warmed to rt and stirred for 30 min. After completion, the reaction was diluted with sodium thiosulfate solution while stirred. The mixture was then extracted with ethyl acetate (2×5 mL). The combined organics were dried with anhydrous sodium sulfate, filtered, and concentrated under reduced pressure. The residue was purified by silica gel column chromatography, eluted with hexanes and ethyl acetate to provide the desired product, which was further purified by silica gel column chromatography, eluted with DCM and EtOAc to provide the desired product as a light-yellow solid (76 mg, 75%). LCMS calculated for C₂₀H₂₇BrN₃O₃Si (M+H)⁺ m/z=464.1; found 464.1.

### Step 12. 3,6-Dimethyl-8-((2-(trimethylsilyl) ethoxy) methoxy)-2-vinyl-8,9-dihydrobenzo[de]pyrazolo[4,5,1-ij][1,7]naphthyridin-4(3H)-one

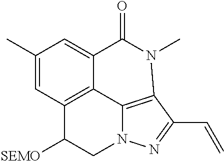

The mixture of 2-bromo-3,6-dimethyl-8-((2-(trimethylsilyl) ethoxy) methoxy)-8,9-dihydrobenzo[de]pyrazolo[4,5,1-ij][1,7]naphthyridin-4(3H)-one (76 mg, 0.16 mmol), PdCl₂(PPh₃)₂ (14 mg, 0.02 mmol), and tributyl(vinyl)stannane (63 mg, 0.2 mmol) in dioxane (2 mL) was heated at 100° C. for 2 h under nitrogen atmosphere. Upon cooling to room temperature, the mixture was diluted with water and extracted with EtOAc. The combined organics were washed with sat. NaCl, dried with anhydrous sodium sulfate, filtered, and concentrated under reduced pressure. The residue was purified by silica gel column chromatography, eluted with DCM and ethyl acetate to provide the desired product as a brown solid (59 mg, 90%). LCMS calculated for C₂₂H₃₀N₃O₃Si (M+H)⁺ m/z=412.2; found 412.2.

### Step 13. 2-Ethyl-3,6-dimethyl-8-((2-(trimethylsilyl) ethoxy) methoxy)-8,9-dihydrobenzo[de]pyrazolo[4,5,1-ij][1,7]naphthyridin-4(3H)-one

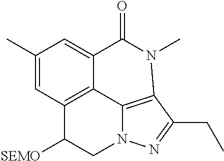

In a 2 mL drum vial, 3,6-dimethyl-8-((2-(trimethylsilyl) ethoxy) methoxy)-2-vinyl-8,9-dihydrobenzo[de]pyrazolo[4,5,1-ij][1,7]naphthyridin-4(3H)-one (59 mg, 0.14 mmol) and Pd/C (10 wt % on active carbon, 10 mg) was dissolved in MeOH (5 mL). The vial was then charged with 1 atm H₂ and stirred for 8 h. The reaction mixture was then filtrate through a short pad of celite and solvent was removed under vacuum to afford the crude product which was used in next step without further purification.

### Step 14. 2-Ethyl-8-hydroxy-3,6-dimethyl-8,9-dihydrobenzo[de]pyrazolo[4,5,1-ij][1,7]naphthyridin-4(3H)-one

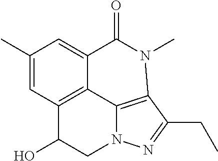 [SMILES: CCc1nn2c3c4c(cc(C)cc4c(=O)n(C)c13)C(O)C2]

In a 2 mL drum vial, 2-ethyl-3,6-dimethyl-8-((2-(trimethylsilyl) ethoxy) methoxy)-8,9-dihydrobenzo[de]pyrazolo[4,5,1-ij][1,7]naphthyridin-4(3H)-one (50 mg, 0.12 mmol) was dissolved in TFA (1 mL). The reaction mixture was stirred for 1 h, and solvent was removed under vacuum. The crude was diluted with sat. NaHCO₃ and extracted with DCM/IPA. The combined organics were washed with sat. NaCl, dried with anhydrous sodium sulfate, filtered, and concentrated under reduced pressure. The residue was purified by silica gel column chromatography, eluted with DCM and MeOH to provide the desired product as an oil (27 mg, 80%). LCMS calculated for C₁₆H₁₈N₃O₂ (M+H)⁺ m/z=284.1; found 284.1.

### Step 15. 8-Bromo-2-ethyl-3,6-dimethyl-8,9-dihydrobenzo[de]pyrazolo[4,5,1-ij][1,7]naphthyridin-4(3H)-one

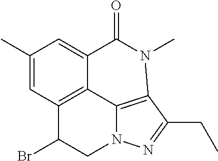 [SMILES: CCc1nn2c3c4c(cc(C)cc4c(=O)n(C)c13)C(Br)C2]

To a mixture of 2-ethyl-8-hydroxy-3,6-dimethyl-8,9-dihydrobenzo[de]pyrazolo[4,5,1-ij][1,7]naphthyridin-4(3H)-one (27 mg, 0.09 mmol) in DCM (2 mL) was added PBr₃ (20 uL, 0.2 mmol) at 0° C. The resulting mixture was warmed to rt and stirred for 2 h. The mixture was then quenched with sat. NaHCO₃ and extracted with DCM (2×). The combined organics were washed with sat. NaCl, and dried with anhydrous sodium sulfate, filtered, and concentrated under reduced pressure. The residue was used directly in next step without further purification.

### Step 16. 8-((2-Bromo-6-methylpyridin-3-yl)amino)-2-ethyl-3,6-dimethyl-8,9-dihydrobenzo[de]pyrazolo[4,5,1-ij][1,7]naphthyridin-4(3H)-one

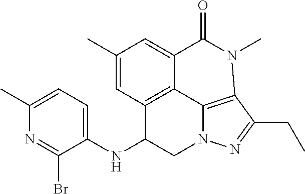 [SMILES: CCc1nn2c3c4c(cc(C)cc4c(=O)n(C)c13)C(Nc1ccc(C)nc1Br)C2]

To the solution of 8-bromo-2-ethyl-3,6-dimethyl-8,9-dihydrobenzo[de]pyrazolo[4,5,1-ij][1,7]naphthyridin-4(3H)-one (24 mg, 0.07 mmol) in DMF (0.3 mL) was added 2-bromo-6-methylpyridin-3-amine (40 mg, 0.2 mmol). The mixture was heated to 80° C. for 2 h. Upon cooling to room temperature, the mixture was diluted with water and extracted with ethyl acetate (3×). The combined organics were washed with water, sat. NaCl, and dried with anhydrous sodium sulfate, filtered, and concentrated under reduced pressure. The residue was purified by silica gel column chromatography, eluted with DCM and MeOH to provide the desired product as a light yellow solid (13 mg, 40%). LCMS calculated for C₂₂H₂₃BrN₅O (M+H)⁺ m/z=452.1; found 452.1.

### Step 17. 2-Ethyl-3,6-dimethyl-8-((6-methyl-[2,4′-bipyridin]-3-yl)amino)-8,9-dihydrobenzo[de]pyrazolo[4,5,1-ij][1,7]naphthyridin-4(3H)-one

The mixture of 8-((2-bromo-6-methylpyridin-3-yl)amino)-2-ethyl-3,6-dimethyl-8,9-dihydrobenzo[de]pyrazolo[4,5,1-ij][1,7]naphthyridin-4(3H)-one (10 mg, 0.02 mmol), Pd(PPh₃)₄ (3 mg, 0.003 mmol), and pyridin-4-ylboronic acid (4 mg, 0.03 mmol) in dioxane (0.5 mL) and water (0.1 mL) was heated at 80° C. for 10 min under nitrogen atmosphere. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₂₇N₆O (M+H)⁺ m/z=451.2; found 451.2.

### Example 41: 9-(1-((6-chloro-1′-methyl-6′-oxo-1′,6′-dihydro-[2,3′-bipyridin]-3-yl)amino)ethyl)-3-ethyl-4,7-dimethyl-3,4-dihydro-5H-pyrazolo[3,4-c]isoquinolin-5-one

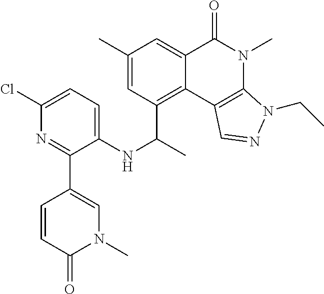 [SMILES: CCn1ncc2c3c(C(C)Nc4ccc(Cl)nc4-c4ccc(=O)n(C)c4)cc(C)cc3c(=O)n(C)c21]

### Step 1. 9-(1-((2-Bromo-6-chloropyridin-3-yl)amino)ethyl)-3-ethyl-4,7-dimethyl-3,4-dihydro-5H-pyrazolo[3,4-c]isoquinolin-5-one

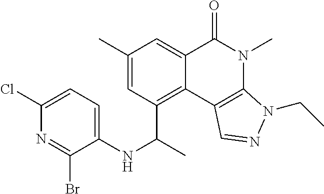 [SMILES: CCn1ncc2c3c(C(C)Nc4ccc(Cl)nc4Br)cc(C)cc3c(=O)n(C)c21]

The titled compound was prepared using similar procedures as described for Example 16 with 2-bromo-6-chloropyridin-3-amine replacing 2-aminobenzoic acid in Step 7 and 1-ethyl-1H-pyrazol-5-amine replacing 1-methyl-1H-pyrazol-5-amine in step 1. Crude product was purified with silica gel chromatography, eluting with 0% to 100% EtOAc in DCM to give the desired product as a beige solid. LCMS calculated for C₂₁H₂₂BrClN₅O (M+H)⁺ m/z=474.1; found 474.

### Step 2. 9-(1-((6-Chloro-1′-methyl-6′-oxo-1′,6′-dihydro-[2,3′-bipyridin]-3-yl)amino)ethyl)-3-ethyl-4,7-dimethyl-3,4-dihydro-5H-pyrazolo[3,4-c]isoquinolin-5-one

The mixture of 9-[1-[(2-bromo-6-chloro-3-pyridyl)amino]ethyl]-3-ethyl-4,7-dimethyl-pyrazolo[3,4-c]isoquinolin-5-one (10.60 mg, 0.02 mmol), 1-methyl-5-(4,4,5,5-tetramethyl-1,3,2-dioxaborolan-2-yl)pyridin-2-one (7.87 mg, 0.03 mmol), potassium carbonate (9.26 mg, 0.07 mmol) and tetrakis(triphenylphosphine)palladium(0) (2.6 mg, 0.002 mmol) in a 5 to 1 mixture of toluene and water (0.2 mL) was heated at 100° C. for 1 h under nitrogen atmosphere. Upon cooling to room temperature, the mixture was concentrated under reduced pressure. The residue was diluted with methanol and purified by prep-HPLC (column: Sunfire prep 15 C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₂₈ClN₆O₂ (M+H)⁺ m/z=503.2; found: 503.2;
The following compounds in Table 4 were prepared similarly as described for Example 41.

TABLE 4

| Ex. | Chemical | | LCMS |
|---|---|---|---|
| # | Name | Structure | m/z (M + H)⁺ |
| 42 | 4-(6-chloro-3-((1-(3-ethyl-4,7- dimethyl-5-oxo-4,5-dihydro-3H- pyrazolo[3,4-c]isoquinolin-9- yl)ethyl)amino)pyridin-2-yl)-2- fluoro-N-methylbenzamide | 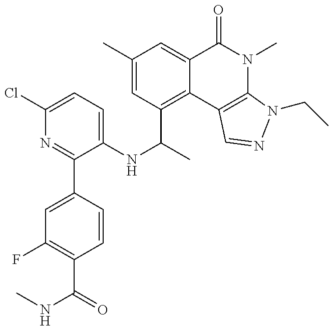 [SMILES: CCn1ncc2c3c(C(C)Nc4ccc(Cl)nc4-c4ccc(C(=O)NC)c(F)c4)cc(C)cc3c(=O)n(C)c21] | 547.2 |
| 43 | 9-(1-((6-chloro-2-(pyrimidin-4- yl)pyridin-3-yl)amino)ethyl)-3- ethyl-4,7-dimethyl-3,4-dihydro-5H- pyrazolo[3,4-c]isoquinolin-5-one | 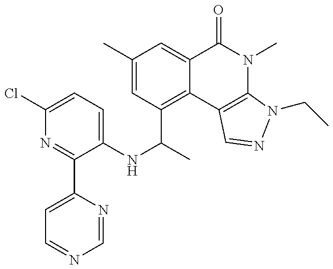 [SMILES: CCn1ncc2c3c(C(C)Nc4ccc(Cl)nc4-c4ccncn4)cc(C)cc3c(=O)n(C)c21] | 474.2 |
| 44 | 9-(1-((6-chloro-2-(pyrimidin-5- yl)pyridin-3-yl)amino)ethyl)-3- ethyl-4,7-dimethyl-3,4-dihydro-5H- pyrazolo[3,4-c]isoquinolin-5-one | 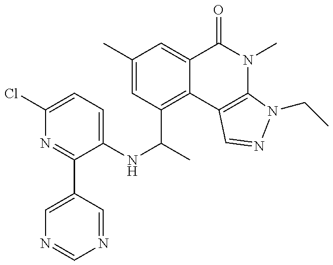 [SMILES: CCn1ncc2c3c(C(C)Nc4ccc(Cl)nc4-c4cncnc4)cc(C)cc3c(=O)n(C)c21] | 474.2 |
| 45 | 9-(1-((6-chloro-3′-fluoro-[2,4′- bipyridin]-3-yl)amino)ethyl)-3- ethyl-4,7-dimethyl-3,4-dihydro-5H- pyrazolo[3,4-c]isoquinolin-5-one | 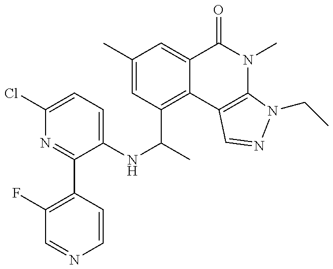 [SMILES: CCn1ncc2c3c(C(C)Nc4ccc(Cl)nc4-c4ccncc4F)cc(C)cc3c(=O)n(C)c21] | 491.2 |
| 46 | 4-(6-chloro-3-((1-(3-ethyl-4,7- dimethyl-5-oxo-4,5-dihydro-3H- pyrazolo[3,4-c]isoquinolin-9- yl)ethyl)amino)pyridin-2-yl)-N,N- dimethylbenzamide | 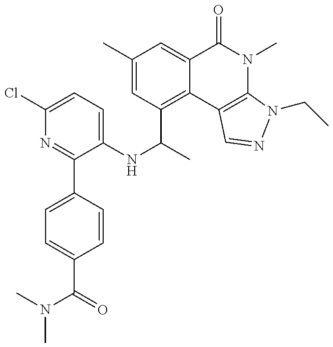 [SMILES: CCn1ncc2c3c(C(C)Nc4ccc(Cl)nc4-c4ccc(C(=O)N(C)C)cc4)cc(C)cc3c(=O)n(C)c21] | 543.2 |
| 47 | 4-(6-chloro-3-((1-(3-ethyl-4,7- dimethyl-5-oxo-4,5-dihydro-3H- pyrazolo[3,4-c]isoquinolin-9- yl)ethyl)amino)pyridin-2-yl) benzoic acid | 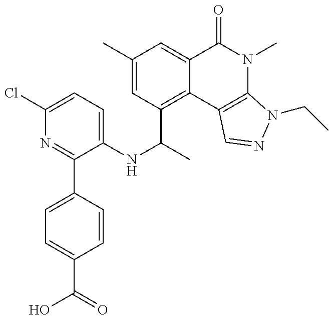 [SMILES: CCn1ncc2c3c(C(C)Nc4ccc(Cl)nc4-c4ccc(C(=O)O)cc4)cc(C)cc3c(=O)n(C)c21] | 516.2 |
| 48 | 9-(1-((6-chloro-2-(1-methyl-1H- pyrazol-5-yl)pyridin-3- yl)amino)ethyl)-3-ethyl-4,7- dimethyl-3,4-dihydro-5H- pyrazolo[3,4-c]isoquinolin-5-one | 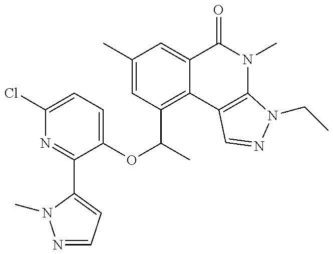 [SMILES: CCn1ncc2c3c(C(C)Oc4ccc(Cl)nc4-c4ccnn4C)cc(C)cc3c(=O)n(C)c21] | 476.2 |
| 49 | 9-(1-((6-chloro-2-(1,5-dimethyl- 1H-pyrazol-4-yl)pyridin-3- yl)amino)ethyl)-3-ethyl-4,7- dimethyl-3,4-dihydro-5H- pyrazolo[3,4-c]isoquinolin-5-one | 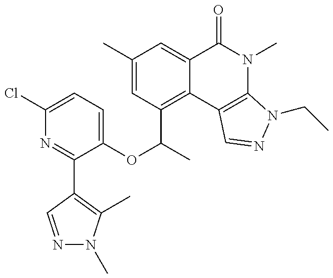 [SMILES: CCn1ncc2c3c(C(C)Oc4ccc(Cl)nc4-c4cnn(C)c4C)cc(C)cc3c(=O)n(C)c21] | 490.2 |
| 50 | 9-(1-((6-chloro-2-(1-methyl-1H- pyrazol-4-yl)pyridin-3- yl)amino)ethyl)-3-ethyl-4,7- dimethyl-3,4-dihydro-5H- pyrazolo[3,4-c]isoquinolin-5-one | 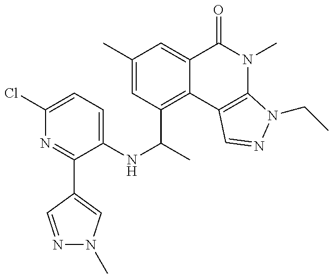 [SMILES: CCn1ncc2c3c(C(C)Nc4ccc(Cl)nc4-c4cnn(C)c4)cc(C)cc3c(=O)n(C)c21] | 476.2 |
| 51 | 9-(1-((6-chloro-2-(1H-pyrazol-4- yl)pyridin-3-yl)amino)ethyl)-3- ethyl-4,7-dimethyl-3,4-dihydro-5H- pyrazolo[3,4-c]isoquinolin-5-one | 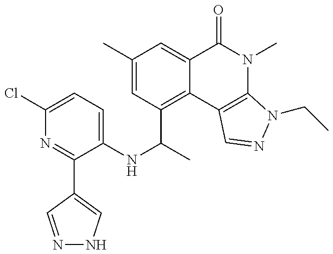 [SMILES: CCn1ncc2c3c(C(C)Nc4ccc(Cl)nc4-c4cn[nH]c4)cc(C)cc3c(=O)n(C)c21] | 462.2 |

### Example 52. 3-Ethyl-4,7-dimethyl-9-(1-((6-methyl-2-(1-methyl-1H-pyrazol-4-yl)pyridin-3-yl)amino)ethyl)-3,4-dihydro-5H-pyrazolo[3,4-c]isoquinolin-5-one

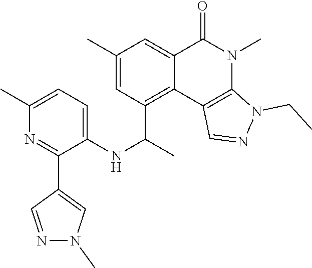 [SMILES: CCn1ncc2c3c(C(C)Nc4ccc(C)nc4-c4cnn(C)c4)cc(C)cc3c(=O)n(C)c21]

### Step 1. 9-(1-((2-Bromo-6-methylpyridin-3-yl)amino)ethyl)-3-ethyl-4,7-dimethyl-3,4-dihydro-5H-pyrazolo[3,4-c]isoquinolin-5-one

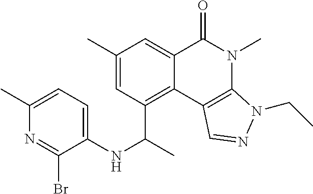 [SMILES: CCn1ncc2c3c(C(C)Nc4ccc(C)nc4Br)cc(C)cc3c(=O)n(C)c21]

The titled compound was prepared using similar procedures as described for Example 16 with 1-ethyl-1H-pyrazol-5-amine replacing 1-methyl-1H-pyrazol-5-amine in Step 1 and 2-bromo-6-methylpyridin-3-amine replacing 2-aminobenzoic acid in Step 7. The resulting mixture was diluted with water and extracted with EtOAc. The combined organics were washed with sat. NaCl, dried with anhydrous sodium sulfate, filtered, and concentrated under reduced pressure. The residue was purified by silica gel column chromatography, eluted with DCM and ethyl acetate to provide the desired product as a white solid (248 mg, 83%). LCMS calculated for C₂₂H₂₅BrN₅O (M+H)⁺ m/z=454.1; found 454.1.

### Step 2: 3-Ethyl-4,7-dimethyl-9-(1-((6-methyl-2-(1-methyl-1H-pyrazol-4-yl)pyridin-3-yl)amino)ethyl)-3,4-dihydro-5H-pyrazolo[3,4-c]isoquinolin-5-one

The mixture of 9-(1-((2-bromo-6-methylpyridin-3-yl)amino)ethyl)-3-ethyl-4,7-dimethyl-3,4-dihydro-5H-pyrazolo[3,4-c]isoquinolin-5-one (10 mg, 0.02 mmol), 1-methyl-4-(4,4,5,5-tetramethyl-1,3,2-dioxaborolan-2-yl)-1H-pyrazole (9 mg, 0.04 mml), Cs₂CO₃ (14 mg, 0.04 mmol) and 1,1′-Bis(diphenylphosphino)ferrocene dichloropalladium (II) (1.6 mg, 0.002 mmol) in dioxane (0.5 mL) and water (0.1 mL) was heated at 80° C. for 1 h under nitrogen atmosphere. The mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₃₀N₇O (M+H)⁺ m/z=456.3; found 456.3.

The following compounds in Table 5 were prepared similarly as described for Example 52.

TABLE 5

| Ex. | Chemical | | LCMS |
|---|---|---|---|
| # | Name | Structure | m/z (M + H)⁺ |
| 53 | 3-ethyl-4,7-dimethyl-9-(1-((6- methyl-2-(1H-pyrazol-4- yl)pyridin-3-yl)amino)ethyl)- 3,4-dihydro-5H-pyrazolo[3,4- c]isoquinolin-5-one | 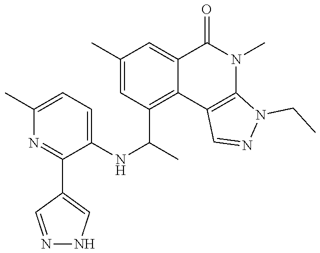 [SMILES: CCn1ncc2c3c(C(C)Nc4ccc(C)nc4-c4cn[nH]c4)cc(C)cc3c(=O)n(C)c21] | 442.2 |
| 54 | 3-ethyl-4,7-dimethyl-9-(1-((6- methyl-2-(1-methyl-1H- pyrazol-5-yl)pyridin-3- yl)amino)ethyl)-3,4-dihydro- 5H-pyrazolo[3,4- c]isoquinolin-5-one | 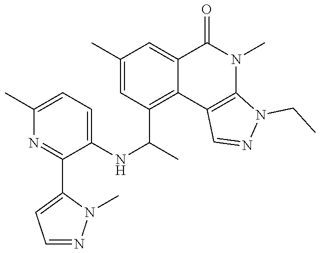 [SMILES: CCn1ncc2c3c(C(C)Nc4ccc(C)nc4-c4ccnn4C)cc(C)cc3c(=O)n(C)c21] | 456.2 |
| 55 | 4-(3-((1-(3-ethyl-4,7- dimethyl-5-oxo-4,5-dihydro- 3H-pyrazolo[3,4- c]isoquinolin-9- yl)ethyl)amino)-6- methylpyridin-2-yl)-2-fluoro- N-methylbenzamide | 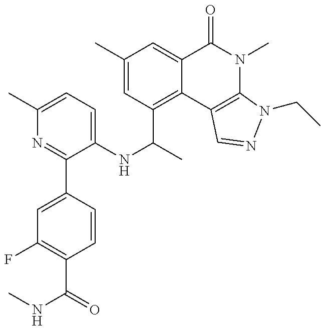 [SMILES: CCn1ncc2c3c(C(C)Nc4ccc(C)nc4-c4ccc(C(=O)NC)c(F)c4)cc(C)cc3c(=O)n(C)c21] | 527.2 |

### Example 56: 3-ethyl-9-(1-((2-(1-(2-hydroxyethyl)-1H-pyrazol-4-yl)phenyl)amino)ethyl)-4,7-dimethyl-3,4-dihydro-5H-pyrazolo[3,4-c]isoquinolin-5-one

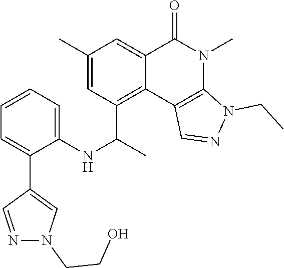 [SMILES: CCn1ncc2c3c(C(C)Nc4ccccc4-c4cnn(CCO)c4)cc(C)cc3c(=O)n(C)c21]

### Step 1. 3-Ethyl-9-(1-((2-iodophenyl)amino)ethyl)-4,7-dimethyl-3,4-dihydro-5H-pyrazolo[3,4-c]isoquinolin-5-one

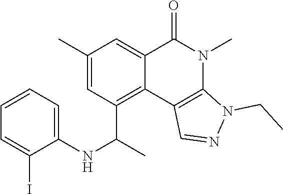 [SMILES: CCn1ncc2c3c(C(C)Nc4ccccc4I)cc(C)cc3c(=O)n(C)c21]

The titled compound was prepared using similar procedures as described for Example 16 with 2-iodoaniline replacing 2-aminobenzoic acid in Step 7 and 1-ethyl-1H-pyrazol-5-amine replacing 1-methyl-1H-pyrazol-5-amine in Step 1. The crude mixture was purified with silica gel chromatography, eluting with 0% to 100% EtOAc in DCM to give the desired product as a white solid. LCMS calculated for C₂₂H₂₄IN₄O (M+H)⁺ m/z=487.1; found 487.1.

### Step 2. 3-Ethyl-9-(1-((2-(1-(2-hydroxyethyl)-1H-pyrazol-4-yl)phenyl)amino)ethyl)-4,7-dimethyl-3,4-dihydro-5H-pyrazolo[3,4-c]isoquinolin-5-one

The mixture of 3-ethyl-9-(1-((2-iodophenyl)amino)ethyl)-4,7-dimethyl-3,4-dihydro-5H-pyrazolo[3,4-c]isoquinolin-5-one (10.0 mg, 0.02 mmol), Pd(dppf)Cl₂·DCM (2.0 mg, 2 μmol), (1-(2-hydroxyethyl)-1H-pyrazol-4-yl) boronic acid (4.4 mg, 0.03 mmol) and cesium carbonate (22.7 mg, 0.07 mmol) in a 5 to 1 mixture of toluene and water (0.2 mL) was heated at 100° C. for 1 h under nitrogen atmosphere. Upon cooling to room temperature, the mixture was concentrated under reduced pressure. The residue was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₁N₆O₂ (M+H)⁺ m/z=471.2; found: 471.2.

### Example 57. 9-(1-((2-(4-Acetylpiperazin-1-yl)phenyl)amino)ethyl)-3-ethyl-4,7-dimethyl-3,4-dihydro-5H-pyrazolo[3,4-c]isoquinolin-5-one

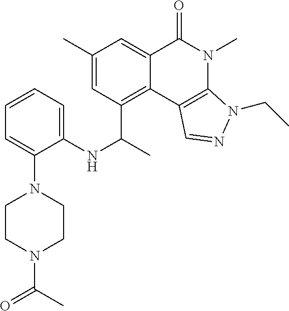 [SMILES: CCn1ncc2c3c(C(C)Nc4ccccc4N4CCN(C(C)=O)CC4)cc(C)cc3c(=O)n(C)c21]

### Step 1. 9-(1-((2-Bromophenyl)amino)ethyl)-3-ethyl-4,7-dimethyl-3,4-dihydro-5H-pyrazolo[3,4-c]isoquinolin-5-one

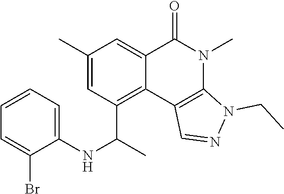 [SMILES: CCn1ncc2c3c(C(C)Nc4ccccc4Br)cc(C)cc3c(=O)n(C)c21]

The titled was prepared using similar procedures as described for Example 16 with 2-bromoaniline replacing 2-aminobenzoic acid in Step 7 and 1-ethyl-1H-pyrazol-5-amine replacing 1-methyl-1H-pyrazol-5-amine in step 1. The crude mixture was purified with silica gel chromatography, eluting with 0% to 100% EtOAc in DCM to give the desired product as a white solid. LCMS calculated for C₂₂H₂₄BrN₄O (M+H)⁺ m/z=439.1; found 439.1;

### Step 2:9-(1-((2-(4-Acetylpiperazin-1-yl)phenyl)amino)ethyl)-3-ethyl-4,7-dimethyl-3,4-dihydro-5H-pyrazolo[3,4-c]isoquinolin-5-one

The mixture of 9-[1-(2-bromoanilino)ethyl]-3-ethyl-4,7-dimethyl-pyrazolo[3,4-c]isoquinolin-5-one (10.20 mg, 0.02 mmol), 1-piperazin-1-ylethanone (4.5 mg, 0.03 mmol), [2-(2-aminophenyl)phenyl]-chloro-palladium; dicyclohexyl-[2-(2,6-diisopropoxyphenyl)phenyl]phosphane (3.61 mg, 0.005 mmol) and potassium carbonate (22.7 mg, 0.07 mmol) in anhydrous 1,4-dioxane (0.2 mL) was heated at 100° C. for overnight under nitrogen. Upon cooling to room temperature, the mixture was concentrated under reduced pressure. The residue was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₃₅N₆O₂ (M+H)⁺ m/z=487.3; found: 487.3.

### Example 58. 9-(1-((2-(5,6-Dihydro-[1,2,4]triazolo[4,3-a]pyrazin-7(8H)-yl)phenyl)amino)ethyl)-3-ethyl-4,7-dimethyl-3,4-dihydro-5H-pyrazolo[3,4-c]isoquinolin-5-one

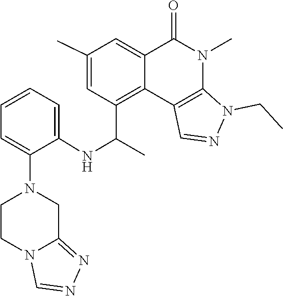 [SMILES: CCn1ncc2c3c(C(C)Nc4ccccc4N4CCn5cnnc5C4)cc(C)cc3c(=O)n(C)c21]

The titled compound was prepared using similar procedures as described for Example 57 with 5,6,7,8-tetrahydro-[1,2,4]triazolo[4,3-a]pyrazine replacing 1-piperazin-1-ylethanone in Step 2. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₁N₈O (M+H)⁺ m/z=483.2; found 483.2.

### Example 59. 3-(6-Chloro-3-((1-(3-ethyl-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one

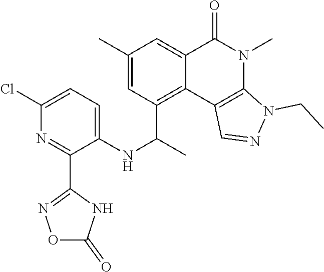

### Step 1. 3-Amino-6-chloro-N-hydroxypicolinimidamide

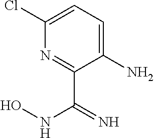 [SMILES: N=C(NO)c1nc(Cl)ccc1N]

The mixture of 3-amino-6-chloropicolinonitrile (5 g, 32.5 mmol) and 50% aqueous hydroxylamine (2 mL, 32.5 mmol) in MeOH (100 mL) was stirred at rt. for 1 h. Then the mixture was poured into water (500 mL). The resulting solid was collected via filtration, washed with water (100 mL), and dried to afford the desired product as a brown solid (5.1 g, 84% yield). LCMS calculated for C₆H₈ClN₄O (M+H)⁺ m/z=187.0; found 187.0.

### Step 2. 3-(3-Amino-6-chloropyridin-2-yl)-1,2,4-oxadiazol-5(4H)-one

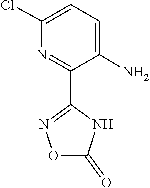 [SMILES: Nc1ccc(Cl)nc1-c1noc(=O)[nH]1]

To a solution of 3-amino-6-chloro-N-hydroxypicolinimidamide (5.1 g, 27.3 mmol) in THF (100 mL) was added 1,1′-Carbonyldiimidazole (4.87 g, 30 mmol) and 1,8-Diazabicyclo[5.4.0]undec-7-ene (16.6 mL, 109 mmol) at 0° C. After stirring for 4 h at rt., the mixture was diluted with 500 mL of water. The aqueous phase was washed with 50 mL ethyl acetate 3 times, then adjusted pH to 7 with 3N HCl. The resulting solid was collected via filtration, washed with water (100 mL), and dried to afford the desired product as a brown solid (4.9 g, 84% yield). LCMS calculated for CH₆ClN₄O₂ (M+H)⁺ m/z=213.0; found 213.0.

### Step 3: 3-(6-Chloro-3-((1-(3-ethyl-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one

The titled compound was prepared using similar procedures as described for Example 16 with 1-ethyl-1H-pyrazol-5-amine replacing 1-methyl-1H-pyrazol-5-amine in Step 1 and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₃ClN₇O₃ (M+H)⁺ m/z=480.2; found 480.2.

### Example 60. 3-(6-Chloro-3-((1-(4-(2,2-difluoroethyl)-3-ethyl-7-methyl-5-oxo-4,5-dihydro-3H-pyrazolo[3,4-c]isoquinolin-9-yl)ethyl)amino)pyridin-2-yl)-1,2,4-oxadiazol-5(4H)-one

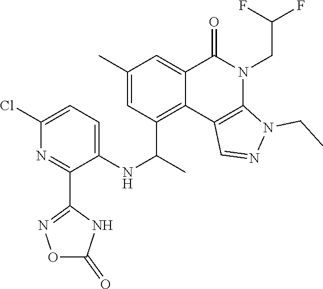 [SMILES: CCn1ncc2c3c(C(C)Nc4ccc(Cl)nc4-c4noc(=O)[nH]4)cc(C)cc3c(=O)n(CC(F)F)c21]

The titled compound was prepared using similar procedures as described for Example 16 with 1-ethyl-1H-pyrazol-5-amine replacing 1-methyl-1H-pyrazol-5-amine in Step 1, 1,1-difluoro-2-iodoethane replacing iodomethane in Step 2 and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₃ClF₂N₇O₃ (M+H)⁺ m/z=530.2; found 530.2.

### Example 61. 3-(6-Chloro-3-((1-(3-cyclopentyl-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one

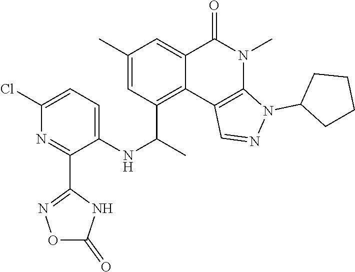

The titled compound was prepared using similar procedures as described for Example 16 with 1-cyclopentyl-1H-pyrazol-5-amine replacing 1-methyl-1H-pyrazol-5-amine in Step 1 and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₂₇ClN₇O₃ (M+H)⁺ m/z=520.2; found 520.2.

### Example 62. 3-(6-Chloro-3-((1-(3-isopropyl-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one

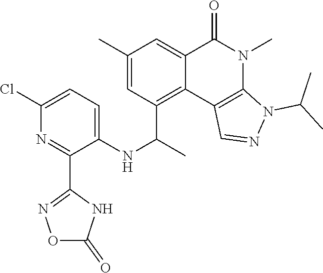

The titled compound was prepared using similar procedures as described for Example 16 with 1-isopropyl-1H-pyrazol-5-amine replacing 1-methyl-1H-pyrazol-5-amine in Step 1 and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₅ClN₇O₃ (M+H)⁺ m/z=494.2; found 494.2.

### Example 63. 3-(6-Chloro-3-((1-(7-chloro-3-ethyl-4-methyl-5-oxo-4,5-dihydro-3H-pyrazolo[3,4-c]isoquinolin-9-yl)ethyl)amino)pyridin-2-yl)-1,2,4-oxadiazol-5(4H)-one

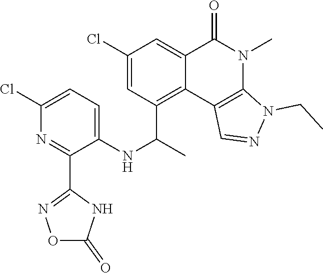

The titled compound was prepared using similar procedures as described for Example 16 with 1-ethyl-1H-pyrazol-5-amine replacing 1-methyl-1H-pyrazol-5-amine in Step 1, 3-bromo-5-chloro-2-iodobenzoic acid replacing 3-bromo-2-iodo-5-methylbenzoic acid in Step 1, and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₂H₂₀C₁₂N₇O₃ (M+H)⁺ m/z=500.1; found 500.1.

### Example 64. 3-(6-Chloro-3-((1-(7-chloro-3-(cyclopropylmethyl)-4-methyl-5-oxo-4,5-dihydro-3H-pyrazolo[3,4-c]isoquinolin-9-yl)ethyl)amino)pyridin-2-yl)-1,2,4-oxadiazol-5(4H)-one

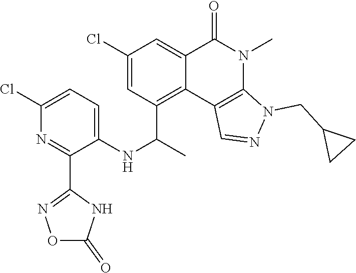 [SMILES: CC(Nc1ccc(Cl)nc1-c1noc(=O)[nH]1)c1cc(Cl)cc2c(=O)n(C)c3c(cnn3CC3CC3)c12]

The titled compound was prepared using similar procedures as described for Example 16 with 1-(cyclopropylmethyl)-1H-pyrazol-5-amine replacing 1-methyl-1H-pyrazol-5-amine in Step 1, 3-bromo-5-chloro-2-iodobenzoic acid replacing 3-bromo-2-iodo-5-methylbenzoic acid in Step 1, and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₂C₁₂N₇O₃ (M+H)⁺ m/z=526.1; found 526.1.

### Example 65. 3-(6-Chloro-3-((1-(4,7-dimethyl-5-oxo-3-propyl-4,5-dihydro-3H-pyrazolo[3,4-c]isoquinolin-9-yl)ethyl)amino)pyridin-2-yl)-1,2,4-oxadiazol-5(4H)-one

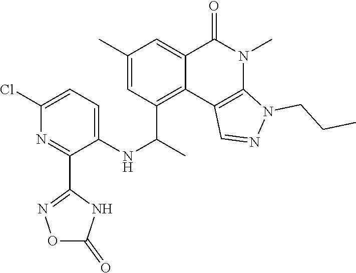

The titled compound was prepared using similar procedures as described for Example 16 with 1-propyl-1H-pyrazol-5-amine replacing 1-methyl-1H-pyrazol-5-amine in Step 1 and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₅ClN₇O₃ (M+H)⁺ m/z=494.2; found 494.2.

### Example 66. 3-(6-Chloro-3-((1-(4,7-dimethyl-5-oxo-3-(tetrahydrofuran-3-yl)-4,5-dihydro-3H-pyrazolo[3,4-c]isoquinolin-9-yl)ethyl)amino)pyridin-2-yl)-1,2,4-oxadiazol-5(4H)-one

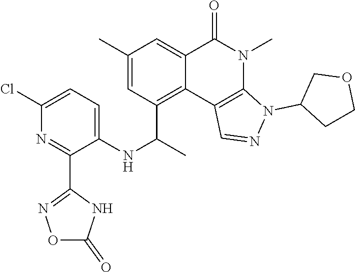

The titled compound was prepared using similar procedures as described for Example 16 with 1-(tetrahydrofuran-3-yl)-1H-pyrazol-5-amine replacing 1-methyl-1H-pyrazol-5-amine in Step 1, and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₂₅ClN₇O₄ (M+H)⁺ m/z=522.2; found 522.2.

### Example 67. 3-(6-Chloro-3-((1-(3-(2-methoxyethyl)-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one

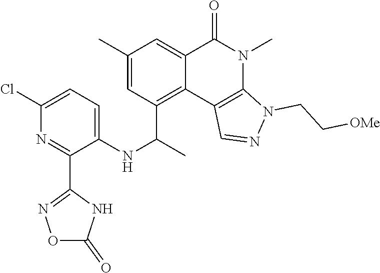 [SMILES: COCCn1ncc2c3c(C(C)Nc4ccc(Cl)nc4-c4noc(=O)[nH]4)cc(C)cc3c(=O)n(C)c21]

The titled compound was prepared using similar procedures as described for Example 16 with 1-(2-methoxyethyl)-1H-pyrazol-5-amine replacing 1-methyl-1H-pyrazol-5-amine in Step 1 and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₅ClN₇O₄ (M+H)⁺ m/z=510.2; found 510.2.

### Example 68. 3-(6-Chloro-3-((1-(3-(cyclopropylmethyl)-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one

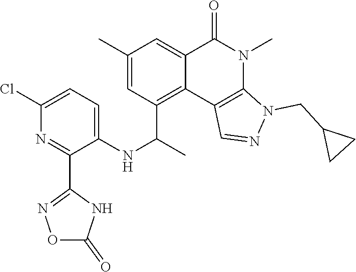 [SMILES: Cc1cc(C(C)Nc2ccc(Cl)nc2-c2noc(=O)[nH]2)c2c(c1)c(=O)n(C)c1c2cnn1CC1CC1]

The titled compound was prepared using similar procedures as described for Example 16 with 1-(cyclopropylmethyl)-1H-pyrazol-5-amine replacing 1-methyl-1H-pyrazol-5-amine in Step 1 and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₂₅ClN₇O₃ (M+H)⁺ m/z=506.2; found 506.2.

### Example 69. 3-(6-Chloro-3-((1-(3-ethyl-4-methyl-5-oxo-7-(trifluoromethyl)-4,5-dihydro-3H-pyrazolo[3,4-c]isoquinolin-9-yl)ethyl)amino)pyridin-2-yl)-1,2,4-oxadiazol-5(4H)-one

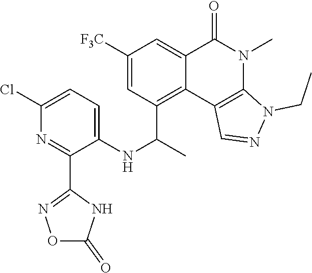 [SMILES: CCn1ncc2c3c(C(C)Nc4ccc(Cl)nc4-c4noc(=O)[nH]4)cc(C(F)(F)F)cc3c(=O)n(C)c21]

The titled compound was prepared using similar procedures as described for Example 16 with 3-bromo-2-iodo-5-(trifluoromethyl)benzoic acid replacing 3-bromo-2-iodo-5-methylbenzoic acid in Step 1, 1-ethyl-1H-pyrazol-5-amine replacing 1-methyl-1H-pyrazol-5-amine in Step 1, and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₀ClF₃N₇O₃ (M+H)⁺ m/z=534.1; found 534.1.

### Example 70. 3-(6-Chloro-3-((1-(3,8-dimethyl-6-oxo-4,5-dihydro-3H,6H-2,2a,5a-triazaaceanthrylen-10-yl)ethyl)amino)pyridin-2-yl)-1,2,4-oxadiazol-5(4H)-one

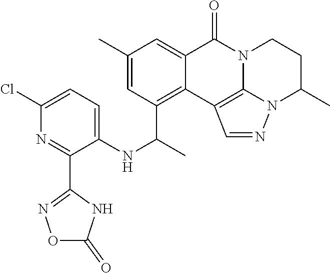 [SMILES: Cc1cc(C(C)Nc2ccc(Cl)nc2-c2noc(=O)[nH]2)c2c(c1)c(=O)n1c3c2cnn3C(C)CC1]

The titled compound was prepared using similar procedures as described for Example 36 with 7-methyl-4,5,6,7-tetrahydropyrazolo[1,5-a]pyrimidine replacing 4,5,6,7-tetrahydropyrazolo[1,5-a]pyrimidine in Step 2 and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₃ClN₇O₃ (M+H)⁺ m/z=492.2; found 492.2.

### Example 71. 3-(6-Chloro-3-((1-(3-ethyl-4,7-dimethyl-5-oxo-4,5-dihydroimidazo[1,5-a]quinazolin-9-yl)ethyl)amino)pyridin-2-yl)-1,2,4-oxadiazol-5(4H)-one

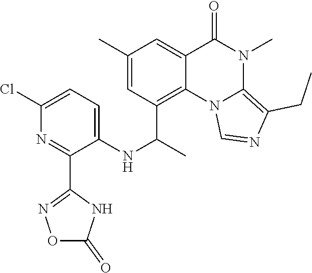 [SMILES: CCc1ncn2c3c(C(C)Nc4ccc(Cl)nc4-c4noc(=O)[nH]4)cc(C)cc3c(=O)n(C)c12]

### Step 1. 9-Acetyl-3-iodo-4,7-dimethylimidazo[1,5-a]quinazolin-5(4H)-one

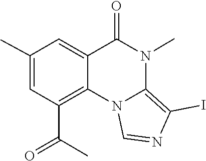 [SMILES: CC(=O)c1cc(C)cc2c(=O)n(C)c3c(I)ncn3c12]

tert-Butyl 9-acetyl-4,7-dimethyl-5-oxo-4,5-dihydroimidazo[1,5-a]quinazoline-3-carboxylate (Example 4, Step 7) (3.0 g, 8.4 mmol) was dissolved in TFA (20 mL). The mixture was stirred for 3 h at rt. After completion, TFA was removed under vacuum. 200 mL of ice water and 50 mL of EtOAc were added to the reaction residue while stirring. After stirring for 20 min, the resulting solid was collected to afford a light brown solid, which was washed with water (2×). The solid was dried under vacuum to provide a beige solid. The solid product was dissolved in DMF (20 mL). To this mixture was added NaHCO₃ (742 mg, 8.8 mmol) at 0° C., after stirring for 5 min, NIS (600 mg, 2.6 mmol) was added in one portion. The resulting mixture was warmed to rt and stirred for 2 h under vacuum. After completion, the reaction was quenched with sat. Na₂S₂O₃ and diluted with water. The resulting solid was collected to afford a light brown solid, which was washed with water (2×). The solid was dried under vacuum to provide a beige solid. LCMS calculated for C₁₄H₁₃IN₃O₂ (M+H)⁺ m/z=382.0; found 382.0.

### Step 2: 9-Acetyl-4,7-dimethyl-3-vinylimidazo[1,5-a]quinazolin-5(4H)-one

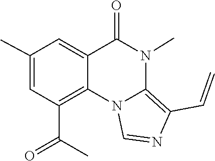 [SMILES: C=Cc1ncn2c3c(C(C)=O)cc(C)cc3c(=O)n(C)c12]

The mixture of 9-acetyl-3-iodo-4,7-dimethylimidazo[1,5-a]quinazolin-5(4H)-one (2.0 g, 5.2 mmol), PdCl₂(PPh₃)₂ (360 mg, 0.5 mmol), and tributyl(vinyl)stannane (1.9 g, 6.0 mmol) in dioxane (20 mL) was heated at 100° C. for 2 h under nitrogen atmosphere. Upon cooling to room temperature, the mixture was diluted with water and extracted with EtOAc. The combined organics were washed with sat. NaCl, dried with anhydrous sodium sulfate, filtered, and concentrated under reduced pressure. The residue was purified by silica gel column chromatography, eluted with DCM and ethyl acetate to provide the desired product as a brown solid (1.3 g, 90%). LCMS calculated for C₁₆H₁₆N₃O₂ (M+H)⁺ m/z=282.1; found 282.2.

### Step 3. 9-Acetyl-3-ethyl-4,7-dimethylimidazo[1,5-a]quinazolin-5(4H)-one

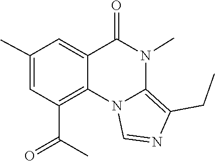 [SMILES: CCc1ncn2c3c(C(C)=O)cc(C)cc3c(=O)n(C)c12]

In a 250 mL flask, 9-acetyl-4,7-dimethyl-3-vinylimidazo[1,5-a]quinazolin-5(4H)-one (1.3 g, 4.6 mmol) and Pd/C (10 wt % on active carbon, 200 mg) was dissolved in MeOH (150 mL). The flask was then charged with 1 atm H₂ and stirred for 18 h. The reaction mixture was then filtrate through a short pad of celite and solvent was removed under vacuum to afford the crude product which was used in next step without further purification.

### Step 4. 3-Ethyl-9-(1-hydroxyethyl)-4,7-dimethylimidazo[1,5-a]quinazolin-5(4H)-one

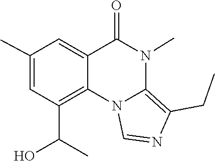 [SMILES: CCc1ncn2c3c(C(C)O)cc(C)cc3c(=O)n(C)c12]

9-Acetyl-3-ethyl-4,7-dimethylimidazo[1,5-a]quinazolin-5(4H)-one (1.0 g, 3.5 mmol) was dissolved in MeOH (100 mL) and DCM (100 mL) then NaBH₄ (182 mg, 4.8 mmol) was added at 0° C. in three portions under nitrogen. The resulting mixture was stirred at same temperature for 10 min before quenched with sat. NH₄Cl. The mixture was diluted with water and extracted with DCM. The combined organics were washed with sat. NaCl, and dried with anhydrous sodium sulfate, filtered, and concentrated under reduced pressure. The residue was purified by silica gel column chromatography, eluted with DCM and MeOH (0 to 4%) to provide the desired product as a light yellow solid (798 mg, 80%). LCMS calculated for C₁₆H₂₀N₃O₂ (M+H)⁺ m/z=286.2; found 286.2.

### Step 5. 9-(1-Bromoethyl)-3-ethyl-4,7-dimethylimidazo[1,5-a]quinazolin-5(4H)-one

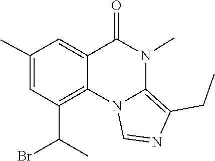 [SMILES: CCc1ncn2c3c(C(C)Br)cc(C)cc3c(=O)n(C)c12]

To a mixture of 3-ethyl-9-(1-hydroxyethyl)-4,7-dimethylimidazo[1,5-a]quinazolin-5(4H)-one (100 mg, 0.35 mmol) in DCM (200 mL) was added PBr₃ (57 uL, 0.6 mmol) at 0° C. The resulting mixture was warmed to rt and stirred for 2 h. The mixture was then quenched with sat. NaHCO₃ and extracted with DCM (2×). The combined organics were washed with sat. NaCl, and dried with anhydrous sodium sulfate, filtered, and concentrated under reduced pressure. The residue was used directly in next step without further purification.

### Step 6. 3-(6-Chloro-3-((1-(3-ethyl-4,7-dimethyl-5-oxo-4,5-dihydroimidazo[1,5-a]quinazolin-9-yl)ethyl)amino)pyridin-2-yl)-1,2,4-oxadiazol-5(4H)-one

To the solution of 9-(1-bromoethyl)-3-ethyl-4,7-dimethylimidazo[1,5-a]quinazolin-5(4H)-one (10 mg, 0.03 mmol) in DMF (0.3 mL) was added 3-(3-amino-6-chloropyridin-2-yl)-1,2,4-oxadiazol-5(4H)-one (20 mg, 0.09 mmol). The mixture was heated to 80° C. for 2 h. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₃ClN₇O₃ (M+H)⁺ m/z=480.2; found 480.2.

### Example 72. 9-(1-((2-(1H-Tetrazol-5-yl)pyridin-3-yl)amino)ethyl)-3-ethyl-4,7-dimethyl-3,4-dihydro-5H-pyrazolo[3,4-c]isoquinolin-5-one

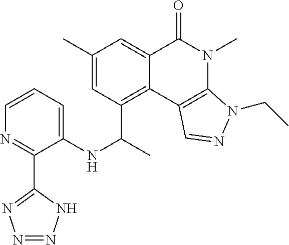 [SMILES: CCn1ncc2c3c(C(C)Nc4cccnc4-c4nnn[nH]4)cc(C)cc3c(=O)n(C)c21]

### Step 1. 2-(1H-Tetrazol-5-yl)pyridin-3-amine

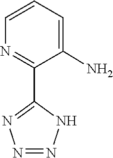 [SMILES: Nc1cccnc1-c1nnn[nH]1]

To a solution of 3-aminopicolinonitrile (1.18 g, 10 mmol) in DMAc (20 mL) was added sodium azide (1.30 g, 20 mmol) and ammonium chloride (1.07 g, 20 mmol) in one portion. The suspension was heated to 120° C., stirred overnight and cooled to rt. The solvent was removed under reduced pressure. The residue was dissolved in 10 mL water, neutralized with 10% HCl and filtered off. The precipitate was washed with water and dried to afford the desired product as a yellow solid (1.0 g, 64% yield). LCMS calculated for C₆H₇N₆ (M+H)⁺ m/z=163.1; found 163.1.

### Step 2. 9-(1-((2-(1H-Tetrazol-5-yl)pyridin-3-yl)amino)ethyl)-3-ethyl-4,7-dimethyl-3,4-dihydro-5H-pyrazolo[3,4-c]isoquinolin-5-one

The titled compound was prepared using similar procedures as described for Example 16 with 1-ethyl-1H-pyrazol-5-amine replacing 1-methyl-1H-pyrazol-5-amine in Step 1 and 2-(1H-tetrazol-5-yl)pyridin-3-amine replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₂H₂₄N₉O (M+H)⁺ m/z=430.2; found 430.2.

### Example 73. 6-Chloro-3-((1-(3-ethyl-4,7-dimethyl-5-oxo-4,5-dihydro-3H-pyrazolo[3,4-c]isoquinolin-9-yl)ethyl)amino)-N-(1-methylpiperidin-4-yl)picolinamide

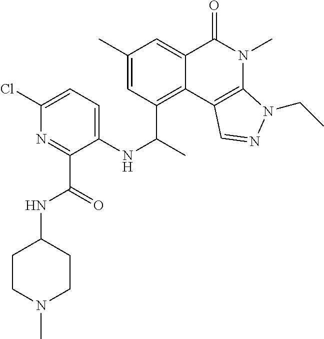 [SMILES: CCn1ncc2c3c(C(C)Nc4ccc(Cl)nc4C(=O)NC4CCN(C)CC4)cc(C)cc3c(=O)n(C)c21]

To a solution of 6-chloro-3-((1-(3-ethyl-4,7-dimethyl-5-oxo-4,5-dihydro-3H-pyrazolo[3,4-c]isoquinolin-9-yl)ethyl)amino)picolinic acid (Example 19) (7 mg, 0.016 mmol) in DMF (1 ml) was added HATU (9 mg, 0.023 mmol) at room temperature. The resulting mixture was stirred for 15 minutes then 1-methylpiperidin-4-amine hydrochloride (3.5 mg, 0.023 mmol) and triethylamine (11.1 uL, 0.08 mmol) were added sequentially. The mixture was stirred at room temperature for 2 hours. The mixture was diluted MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a yellow solid. LCMS calculated for C₂₈H₃₅ClN₇O₂ (M+H)⁺ m/z=536.2; found 536.2.

The following compounds in Table 6 were prepared similarly as described for Example 73.

TABLE 6

| Ex. | Chemical | | LCMS |
|---|---|---|---|
| # | Name | Structure | m/z (M + H)⁺ |
| 74 | 6-chloro-3-((1-(3-ethyl-4,7-dimethyl- 5-oxo-4,5-dihydro-3H-pyrazolo[3,4- c]isoquinolin-9-yl)ethyl)amino)-N- (pyridin-4-yl)picolinamide | 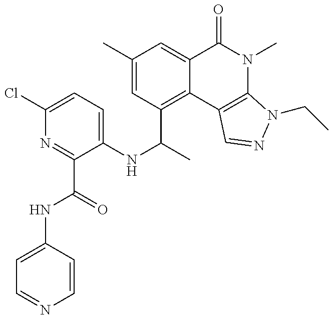 [SMILES: CCn1ncc2c3c(C(C)Nc4ccc(Cl)nc4C(=O)Nc4ccncc4)cc(C)cc3c(=O)n(C)c21] | 516.2 |
| 75 | 6-chloro-3-((1-(3-ethyl-4,7-dimethyl- 5-oxo-4,5-dihydro-3H-pyrazolo[3,4- c]isoquinolin-9-yl)ethyl)amino)-N- methoxypicolinamide | 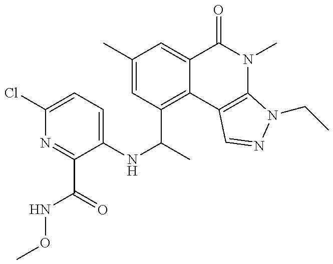 [SMILES: CCn1ncc2c3c(C(C)Nc4ccc(Cl)nc4C(=O)NOC)cc(C)cc3c(=O)n(C)c21] | 469.2 |

### Example 76. 9-(1-((6-Chloro-3′-fluoro-[2,4′-bipyridin]-3-yl)amino)-2-hydroxyethyl)-3-ethyl-4,7-dimethyl-3,4-dihydro-5H-pyrazolo[3,4-c]isoquinolin-5-one

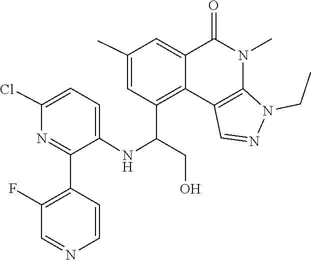 [SMILES: CCn1ncc2c3c(C(CO)Nc4ccc(Cl)nc4-c4ccncc4F)cc(C)cc3c(=O)n(C)c21]

### Step 1. 9-Bromo-3-ethyl-4,7-dimethyl-3,4-dihydro-5H-pyrazolo[3,4-c]isoquinolin-5-one

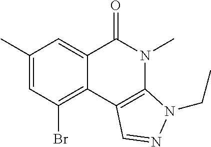 [SMILES: CCn1ncc2c3c(Br)cc(C)cc3c(=O)n(C)c21]

The titled compound was prepared using similar procedures as described for Example 16 with 1-ethyl-1H-pyrazol-5-amine replacing 1-methyl-1H-pyrazol-5-amine in Step 1. The resulting mixture was purified with silica gel chromatography, eluting with 0% to 100% EtOAc in DCM to give the desired product as a beige solid. LCMS calculated for C₁₄H₁₅BrN₃O (M+H)⁺ m/z=320.0; found 320.0.

### Step 2. 3-Ethyl-4,7-dimethyl-9-vinyl-3,4-dihydro-5H-pyrazolo[3,4-c]isoquinolin-5-one

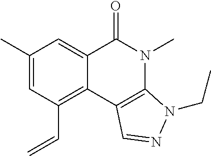 [SMILES: C=Cc1cc(C)cc2c(=O)n(C)c3c(cnn3CC)c12]

The mixture of 9-bromo-3-ethyl-4,7-dimethyl-3,4-dihydro-5H-pyrazolo[3,4-c]isoquinolin-5-one (500 mg, 1.6 mmol), PdCl₂(PPh₃)₂ (110 mg, 0.16 mmol), and tributyl (vinyl)stannane (500 mg, 1.6 mmol) in dioxane (5 mL) was heated at 100° C. for 2 h under nitrogen atmosphere. Upon cooling to room temperature, the mixture was diluted with water and extracted with EtOAc. The combined organics were washed with sat. NaCl, dried with anhydrous sodium sulfate, filtered, and concentrated under reduced pressure. The residue was purified by silica gel column chromatography, eluted with DCM and ethyl acetate to provide the desired product as a brown solid. LCMS calculated for C₁₆H₁₈N₃O (M+H)⁺ m/z=268.0; found 268.1.

### Step 3. 3-Ethyl-4,7-dimethyl-9-(oxiran-2-yl)-3,4-dihydro-5H-pyrazolo[3,4-c]isoquinolin-5-one

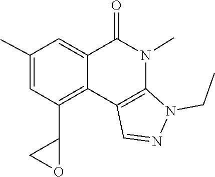 [SMILES: CCn1ncc2c3c(C4CO4)cc(C)cc3c(=O)n(C)c21]

To a mixture of 3-ethyl-4,7-dimethyl-9-vinyl-3,4-dihydro-5H-pyrazolo[3,4-c]isoquinolin-5-one (400 mg, 1.50 mmol) in DCM (50 mL) was added mCPBA (489 mg, 2.0 mmol) at 0° C. in three portions over 1 h. The reaction mixture was then warmed to rt and stirred for 2 h. After completion, the reaction was diluted with ice water and washed with sat NaHCO₃ and sat. NaCl, dried with anhydrous sodium sulfate, filtered, and concentrated under reduced pressure to give the title compound as a light-yellow oil. The crude product was used in the next step without further purification.

### Step 4. 9-(1-((2-Bromo-6-chloropyridin-3-yl)amino)-2-hydroxyethyl)-3-ethyl-4,7-dimethyl-3,4-dihydro-5H-pyrazolo[3,4-c]isoquinolin-5-one

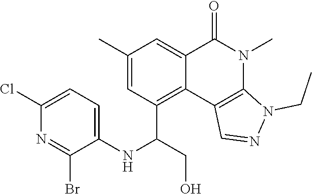 [SMILES: CCn1ncc2c3c(C(CO)Nc4ccc(Cl)nc4Br)cc(C)cc3c(=O)n(C)c21]

To a mixture of 3-ethyl-4,7-dimethyl-9-(oxiran-2-yl)-3,4-dihydro-5H-pyrazolo[3,4-c]isoquinolin-5-one (100 mg, 0.35 mmol) in acetonitrile (10 mL) was added 2-bromo-6-chloropyridin-3-amine (100 mg, 0.48 mmol) and Sc(OTf)₃ (20 mg, 0.04 mmol) at rt. The reaction mixture was then warmed to rt and stirred for 2 h. After completion, the reaction was diluted with ice water and washed with sat NaHCO₃ and sat. NaCl, dried with anhydrous sodium sulfate, filtered, and concentrated under reduced pressure to give the crude product. The residue was purified by silica gel column chromatography, eluted with DCM and MeOH to provide the desired product as a brown solid. LCMS calculated for C₂₁H₂₂BrClN₅O₂ (M+H)⁺ m/z=490.0; found 490.1.

### Step 5. 9-(1-((6-Chloro-3′-fluoro-[2,4′-bipyridin]-3-yl)amino)-2-hydroxyethyl)-3-ethyl-4,7-dimethyl-3,4-dihydro-5H-pyrazolo[3,4-c]isoquinolin-5-one

The mixture of 9-(1-((2-bromo-6-chloropyridin-3-yl)amino)-2-hydroxyethyl)-3-ethyl-4,7-dimethyl-3,4-dihydro-5H-pyrazolo[3,4-c]isoquinolin-5-one (10 mg, 0.02 mmol), Pd(PPh₃)₄ (3 mg, 0.003 mmol), and (3-fluoropyridin-4-yl) boronic acid (4 mg, 0.03 mmol) in dioxane (0.5 mL) and water (0.1 mL) was heated at 80° C. for 10 min under nitrogen atmosphere. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₂₅ClFN₆O₂ (M+H)⁺ m/z=507.2; found 507.2.

The following compounds in Table 7 were prepared similarly as described for Example 76.

TABLE 7

| Ex. | Chemical | | LCMS |
|---|---|---|---|
| # | Name | Structure | m/z (M + H)⁺ |
| 77 | 9-(1-((6-Chloro-2-(1-methyl-1H- pyrazol-4-yl)pyridin-3-yl)amino)-2- hydroxyethyl)-3-ethyl-4,7- dimethyl-3,4-dihydro-5H- pyrazolo[3,4-c]isoquinolin-5-one | 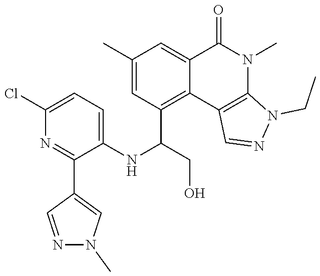 [SMILES: CCn1ncc2c3c(C(CO)Nc4ccc(Cl)nc4-c4cnn(C)c4)cc(C)cc3c(=O)n(C)c21] | 492.2 |
| 78 | 9-(1-((6-Chloro-2-(1,5-dimethyl- 1H-pyrazol-4-yl)pyridin-3- yl)amino)-2-hydroxyethyl)-3-ethyl- 4,7-dimethyl-3,4-dihydro-5H- pyrazolo[3,4-c]isoquinolin-5-one | 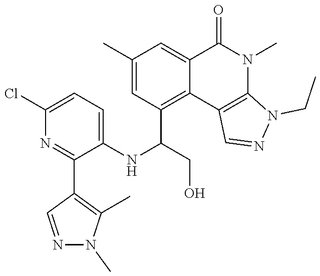 [SMILES: CCn1ncc2c3c(C(CO)Nc4ccc(Cl)nc4-c4cnn(C)c4C)cc(C)cc3c(=O)n(C)c21] | 506.2 |

### Example 79. 9-(1-((6-Chloro-2-(1-(1-methylpiperidin-4-yl)-1H-pyrazol-4-yl)pyridin-3-yl)amino)ethyl)-3-ethyl-4,7-dimethyl-3,4-dihydro-5H-pyrazolo[3,4-c]isoquinolin-5-one

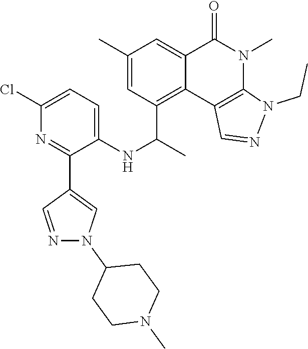 [SMILES: CCn1ncc2c3c(C(C)Nc4ccc(Cl)nc4-c4cnn(C5CCN(C)CC5)c4)cc(C)cc3c(=O)n(C)c21]

The title compound was prepared using similar procedures as described for Example 41 with 1-methyl-4-(4-(4,4,5,5-tetramethyl-1,3,2-dioxaborolan-2-yl)-1H-pyrazol-1-yl)piperidine replacing 1-methyl-5-(4,4,5,5-tetramethyl-1,3,2-dioxaborolan-2-yl)pyridin-2-one in Step 2. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₀H₃₆ClN₈O (M+H)⁺ m/z=559.2; found 559.2.

### Example 80. 9-(1-((2-(4-Acetylpiperazin-1-yl)-6-chloropyridin-3-yl)amino)ethyl)-3-ethyl-4,7-dimethyl-3,4-dihydro-5H-pyrazolo[3,4-c]isoquinolin-5-one

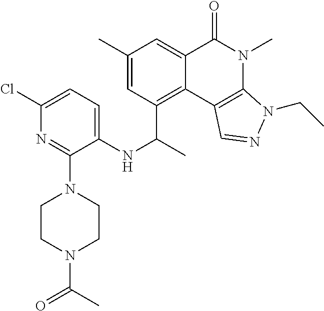 [SMILES: CCn1ncc2c3c(C(C)Nc4ccc(Cl)nc4N4CCN(C(C)=O)CC4)cc(C)cc3c(=O)n(C)c21]

### Step 1: 9-(1-((6-chloro-2-fluoropyridin-3-yl)amino)ethyl)-3-ethyl-4,7-dimethyl-3,4-dihydro-5H-pyrazolo[3,4-c]isoquinolin-5-one

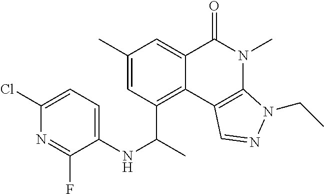 [SMILES: CCn1ncc2c3c(C(C)Nc4ccc(Cl)nc4F)cc(C)cc3c(=O)n(C)c21]

The title compound was prepared using similar procedures as described for Example 16 with 6-chloro-2-fluoropyridin-3-amine replacing 2-aminobenzoic acid in Step 7 and 1-ethyl-1H-pyrazol-5-amine replacing 1-methyl-1H-pyrazol-5-amine in Step 1. The crude product was purified with silica gel chromatography, eluting with 0% to 100% EtOAc in DCM to give the desired product as a beige solid. LCMS calculated for C₂₁H₂₂ClFN₅O (M+H)⁺ m/z=414.1; found 414.

### Step 2: 9-(1-((2-(4-acetylpiperazin-1-yl)-6-chloropyridin-3-yl)amino)ethyl)-3-ethyl-4,7-dimethyl-3,4-dihydro-5H-pyrazolo[3,4-c]isoquinolin-5-one

To a solution of 9-(1-((6-chloro-2-fluoropyridin-3-yl)amino)ethyl)-3-ethyl-4,7-dimethyl-3,4-dihydro-5H-pyrazolo[3,4-c]isoquinolin-5-one (10.0 mg, 0.02 mmol) in anhydrous DMF (0.3 mL) was added N-ethyl-N-isopropyl-propan-2-amine (37.0 uL, 0.22 mmol) and 1-(piperazin-1-yl) ethan-1-one (11.2 uL, 0.09 mmol). The resulting mixture was heated at 100° C. for 3 hours. Upon completion of reaction, the residue was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₃ClN₇O₂ (M+H)⁺ m/z=522.2; found 522.2.

### Example 81: 9-(1-((6-Chloro-2-(1-methyl1H-pyrazol-4-yl)pyridin-3-yl)amino)ethyl)-4,7-dimethyl-3-(1-methylazetidin-3-yl)-3,4-dihydro-5H-pyrazolo[3,4-c]isoquinolin-5-one

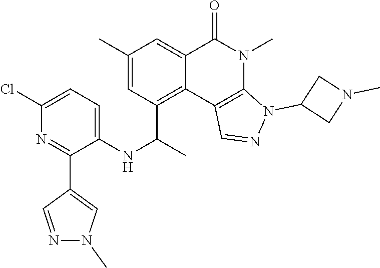 [SMILES: Cc1cc(C(C)Nc2ccc(Cl)nc2-c2cnn(C)c2)c2c(c1)c(=O)n(C)c1c2cnn1C1CN(C)C1]

### Step 1: tert-butyl 3-hydrazineylazetidine-1-carboxylate

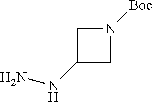 [SMILES: CC(C)(C)OC(=O)N1CC(NN)C1]

To a 500 mL seal flask was added tert-butyl 3-((methylsulfonyl)oxy) azetidine-1-carboxylate (20 g, 79.7 mmol) and hydrazine monohydrate (50 mL). The mixture was then heated at 100° C. for 20 h. After cooled to rt, the reaction mixture was diluted with 50 mL of water and extracted with EtOAc (4×). The combined organics were dried with anhydrous sodium sulfate, filtered, and concentrated under reduced pressure. The residue was used directly in the next step without further purification.

### Step 2: ethyl 5-amino-1-(1-(tert-butoxycarbonyl) azetidin-3-yl)-1H-pyrazole-4-carboxylate

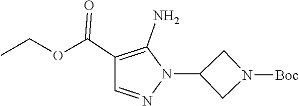 [SMILES: CCOC(=O)c1cnn(C2CN(C(=O)OC(C)(C)C)C2)c1N]

To a 500 mL flask was added tert-butyl 3-hydrazineylazetidine-1-carboxylate (15 g, 80 mmol) and ethyl (E)-2-cyano-3-ethoxyacrylate (16.26 g, 96 mmol) and EtOH (500 mL). The mixture was then heated at 85° C. for 15 h. After cooled to rt, the reaction mixture was concentrated under reduced pressure. The resulting crude product was stirred in 200 mL of DCM. The resulting solid was collected after filtration and used directly in the next step without further purification.

### Step 3: tert-butyl 3-(5-amino-1H-pyrazol-1-yl) azetidine-1-carboxylate

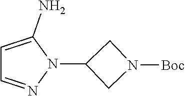 [SMILES: CC(C)(C)OC(=O)N1CC(n2nccc2N)C1]

To a 500 mL flask was added ethyl 5-amino-1-(1-(tert-butoxycarbonyl) azetidin-3-yl)-1H-pyrazole-4-carboxylate (20 g, 64 mmol), EtOH (500 mL) and 5N NaOH solution (100 mL). The mixture was then heated at 85° C. for 2 h. After cooled to rt, the reaction mixture was concentrated under reduced pressure. The resulting residue was acidified by adding 6N HCl slowly at 0° C. The resulting solid was collected after filtration and dried under vacuum to afford a white solid. To the solid was added chlorobenzene (400 mL), and the resulting mixture was heated at 140° C. for 12 h. After completion, the solvent was removed under vacuum and the residue was used directly in next step as crude product.

### Step 4: tert-butyl 3-(5-(3-bromo-2-iodo-5-methylbenzamido)-1H-pyrazol-1-yl) azetidine-1-carboxylate

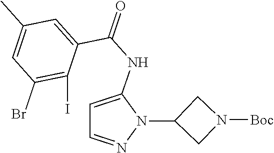 [SMILES: Cc1cc(Br)c(I)c(C(=O)Nc2ccnn2C2CN(C(=O)OC(C)(C)C)C2)c1]

To a 500 mL flask was added tert-butyl 3-(5-amino-1H-pyrazol-1-yl) azetidine-1-carboxylate (12 g, 50 mmol), 3-bromo-2-iodo-5-methylbenzoic acid (19.4 g, 57 mmoL) and THF (500 mL). To a mixture was then added propanephosphonic acid anhydride (50% THF solution, 50 mL) and DIEA (13 mL, 71 mmol). The reaction mixture was then heated at 70° C. overnight. After completion, the solvent was removed under vacuum. The residue was diluted with EtOAc, washed with sat. NaHCO₃ and brine. The combined organic layer was dried over Na₂SO₄ and concentrated under vacuum. The residue was purified by silica gel column chromatography, eluted with DCM and MeOH (0 to 4%) to provide the desired product as a brown solid (14 g, 50%). LCMS calculated for C₁₉H₂₃BrIN₄O₃ (M+H)⁺ m/z=561.0; found 561.0.

### Step 5: tert-butyl 3-(5-(3-bromo-2-iodo-N,5-dimethylbenzamido)-1H-pyrazol-1-yl) azetidine-1-carboxylate

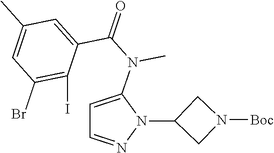 [SMILES: Cc1cc(Br)c(I)c(C(=O)N(C)c2ccnn2C2CN(C(=O)OC(C)(C)C)C2)c1]

To a mixture of tert-butyl 3-(5-(3-bromo-2-iodo-5-methylbenzamido)-1H-pyrazol-1-yl) azetidine-1-carboxylate (14 g, 25 mmol) in DMF (300 mL) was added NaH (1.2 g, 30 mmol) at 0° C. under nitrogen atmosphere. The resulting mixture was stirred at same temperature for 30 min and then MeI (4.3 g, 30 mmol) was added. The reaction mixture was then slowly warmed to rt and stirred for 1 h. After completion, the reaction mixture was carefully quenched with water at 0° C. while vigorously stirred. The mixture was then extracted with ethyl acetate. The combined organics were dried with anhydrous sodium sulfate, filtered, and concentrated under reduced pressure. The residue was purified by silica gel column chromatography, eluted with hexanes and ethyl acetate to provide the desired product, which was further purified by silica gel column chromatography, eluted with DCM and EtOAc to provide the desired product as a white solid (11.4 g, 80%). LCMS calculated for C₂₀H₂₅BrIN₄O₃ (M+H)⁺ m/z=575.1; found 575.0.

### Step 6: tert-butyl 3-(9-bromo-4,7-dimethyl-5-oxo-4,5-dihydro-3H-pyrazolo[3,4-c]isoquinolin-3-yl) azetidine-1-carboxylate

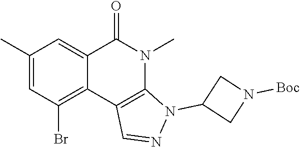 [SMILES: Cc1cc(Br)c2c(c1)c(=O)n(C)c1c2cnn1C1CN(C(=O)OC(C)(C)C)C1]

A mixture of tert-butyl 3-(5-(3-bromo-2-iodo-5-methylbenzamido)-1H-pyrazol-1-yl) azetidine-1-carboxylate (11.4 g, 20 mmol), Pd(PPh₃)₄ (6.9 g, 6 mmol), KOAc (11.7 g, 120 mmol) in DMAc (200 mL) was heated at 105° C. for 24 h under nitrogen atmosphere. Upon cooling to room temperature, the mixture was diluted with water and extracted with EtOAc. The combined organics were washed with sat. NaCl, dried with anhydrous sodium sulfate and filtered. The filtrate was concentrated and used directly in the next step without future purification. LCMS calculated for C₂₀H₂₄BrN₄O₃ (M+H)⁺ m/z=447.1; found 447.1.

### Step 7: tert-butyl 3-(9-acetyl-4,7-dimethyl-5-oxo-4,5-dihydro-3H-pyrazolo[3,4-c]isoquinolin-3-yl) azetidine-1-carboxylate

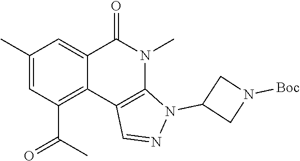 [SMILES: CC(=O)c1cc(C)cc2c(=O)n(C)c3c(cnn3C3CN(C(=O)OC(C)(C)C)C3)c12]

A mixture of tert-butyl 3-(9-bromo-4,7-dimethyl-5-oxo-4,5-dihydro-3H-pyrazolo[3,4-c]isoquinolin-3-yl) azetidine-1-carboxylate (5.3 g, 12 mmol), PdCl₂(PPh₃)₂ (842 mg, 1.2 mmol), and tributyl(1-ethoxyvinyl)stannane (4.4 g, 14 mmol) in dioxane (200 mL) was heated at 100° C. for 8 h under nitrogen atmosphere. Upon cooling to room temperature, to the mixture was added 2 N HCl and stirred for 30 min before the mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a white solid (3.4 g, 70%). LCMS calculated for C₂₂H₂₇N₄O₄ (M+H)⁺ m/z=411.2; found 411.2.

### Step 8: tert-butyl 3-(9-(1-hydroxyethyl)-4,7-dimethyl-5-oxo-4,5-dihydro-3H-pyrazolo[3,4-c]isoquinolin-3-yl) azetidine-1-carboxylate

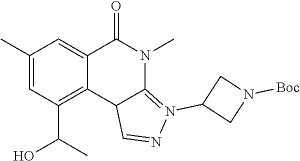

To a mixture of tert-butyl 3-(9-acetyl-4,7-dimethyl-5-oxo-4,5-dihydro-3H-pyrazolo[3,4-c]isoquinolin-3-yl) azetidine-1-carboxylate (3.4 g, 8.3 mmol) in MeOH (100 mL) and DCM (100 mL) was added NaBH₄ (342 mg, 9 mmol) at 0° C. The resulting mixture was stirred at same temperature for 10 min before quenched with sat. NH₄Cl. The mixture was diluted with water and extracted with DCM/IPA 3:1. The combined organics were washed with sat. NaCl and dried with anhydrous sodium sulfate, filtered, and concentrated under reduced pressure. The residue was purified by silica gel column chromatography, eluted with DCM and MeOH to provide the desired product as a white solid (2.8 g, 82%). LCMS calculated for C₂₂H₂₉N₄O₄ (M+H)⁺ m/z=413.2; found 413.2.

### Step 9: tert-butyl 3-(9-(1-bromoethyl)-4,7-dimethyl-5-oxo-4,5-dihydro-3H-pyrazolo[3,4-c]isoquinolin-3-yl) azetidine-1-carboxylate

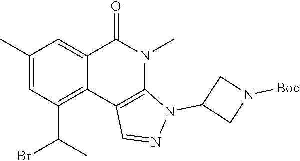 [SMILES: Cc1cc(C(C)Br)c2c(c1)c(=O)n(C)c1c2cnn1C1CN(C(=O)OC(C)(C)C)C1]

To a mixture of tert-butyl 3-(9-(1-hydroxyethyl)-4,7-dimethyl-5-oxo-4,5-dihydro-3H-pyrazolo[3,4-c]isoquinolin-3-yl) azetidine-1-carboxylate (500 mg, 1.2 mmol) in THF (50 mL) was added pyridine (0.29 mL, 3.6 mmol) and PBr₃ (648 mg, 2.4 mmol). The resulting mixture was stirred at rt for 2 h. The mixture was then quenched with sat. NaHCO₃ and extracted with EtOAc. The combined organics were washed with sat. NaCl, dried with anhydrous sodium sulfate, filtered, and concentrated under reduced pressure. The residue was used directly in the next step without further purification.

### Step 10: 3-(azetidin-3-yl)-9-(1-((6-chloro-2-(1-methyl-1H-pyrazol-4-yl)pyridin-3-yl)amino)ethyl)-4,7-dimethyl-3,4-dihydro-5H-pyrazolo[3,4-c]isoquinolin-5-one

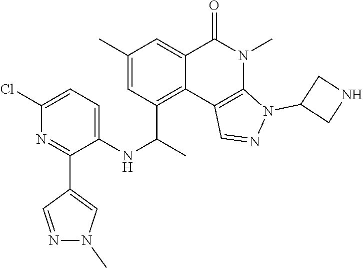 [SMILES: Cc1cc(C(C)Nc2ccc(Cl)nc2-c2cnn(C)c2)c2c(c1)c(=O)n(C)c1c2cnn1C1CNC1]

To tert-butyl 3-(9-(1-bromoethyl)-4,7-dimethyl-5-oxo-4,5-dihydro-3H-pyrazolo[3,4-c]isoquinolin-3-yl) azetidine-1-carboxylate (20 mg, 0.042 mmol) in 2 dram vial was added DMF (0.2 mL), and 6-chloro-2-(1-methyl-1H-pyrazol-4-yl)pyridin-3-amine (20 mg, 0.096 mmol). The mixture was heated to 80° C. for 1 h. Upon cooling to room temperature, TFA (0.5 mL) was added to the mixture. The mixture was stirred for 30 min before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₂₈ClN₈O (M+H)⁺ m/z=503.2; found 503.2.

### Step 11: 9-(1-((6-chloro-2-(1-methyl-1H-pyrazol-4-yl)pyridin-3-yl)amino)ethyl)-4,7-dimethyl-3-(1-methylazetidin-3-yl)-3,4-dihydro-5H-pyrazolo[3,4-c]isoquinolin-5-one

To a mixture of 3-(azetidin-3-yl)-9-(1-((6-chloro-2-(1-methyl-1H-pyrazol-4-yl)pyridin-3-yl)amino)ethyl)-4,7-dimethyl-3,4-dihydro-5H-pyrazolo[3,4-c]isoquinolin-5-one (5 mg, 0.01 mmol) was added DCM (0.5 mL), formaldehyde (water solution, 10 uL) and sodium triacetoxyborohydride (5 mg, 0.023 mmol). The mixture was stirred for 30 min, then diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₀ClN₈O (M+H)⁺ m/z=517.2; found 517.2.

### Examples 82-88

Examples 82-88 in Table 8 were prepared similarly as described for Example 81.

TABLE 8

| Example | Chemical | | LCMS |
|---|---|---|---|
| # | name | Structure | m/z (M + H)⁺ |
| 82 | 9-(1-((6-chloro-2-(1-methyl- 1H-1,2,4-triazol-3-yl)pyridin- 3-yl)amino)ethyl)-4,7- dimethyl-3-(1-methylazetidin- 3-yl)-3,4-dihydro-5H- pyrazolo[3,4-c]isoquinolin-5- one | 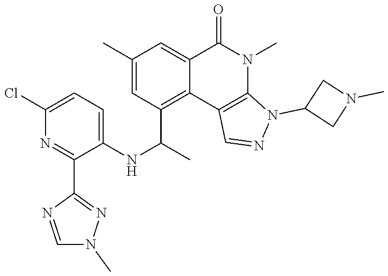 [SMILES: Cc1cc(C(C)Nc2ccc(Cl)nc2-c2ncn(C)n2)c2c(c1)c(=O)n(C)c1c2cnn1C1CN(C)C1] | 518.2 |
| 83 | 9-(1-((6-chloro-2-(1-(2- hydroxyethyl)-1H-1,2,4- triazol-3-yl)pyridin-3- yl)amino)ethyl)-4,7-dimethyl- 3-(1-methylazetidin-3-yl)-3,4- dihydro-5H-pyrazolo[3,4- c]isoquinolin-5-one | 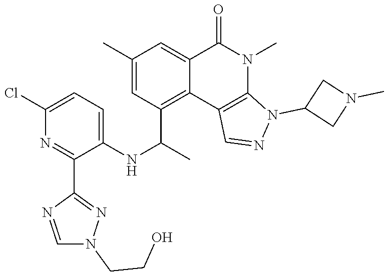 [SMILES: Cc1cc(C(C)Nc2ccc(Cl)nc2-c2ncn(CCO)n2)c2c(c1)c(=O)n(C)c1c2cnn1C1CN(C)C1] | 548.2 |
| 84 | 9-(1-((6-chloro-2-(1H-pyrazol- 4-yl)pyridin-3-yl)amino)ethyl)- 4,7-dimethyl-3-(1- methylazetidin-3-yl)-3,4- dihydro-5H-pyrazolo[3,4- c]isoquinolin-5-one | 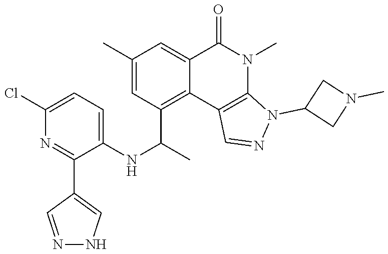 [SMILES: Cc1cc(C(C)Nc2ccc(Cl)nc2-c2cn[nH]c2)c2c(c1)c(=O)n(C)c1c2cnn1C1CN(C)C1] | 503.2 |
| 85 | 9-(1-((6-chloro-1′-methyl-6′- oxo-1′,6′-dihydro-[2,3′- bipyridin]-3-yl)amino)ethyl)- 4,7-dimethyl-3-(1- methylazetidin-3-yl)-3,4- dihydro-5H-pyrazolo[3,4- c]isoquinolin-5-one | 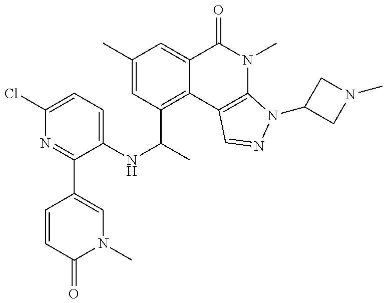 [SMILES: Cc1cc(C(C)Nc2ccc(Cl)nc2-c2ccc(=O)n(C)c2)c2c(c1)c(=O)n(C)c1c2cnn1C1CN(C)C1] | 544.2 |
| 86 | 5-(5-chloro-2-((1-(4,7- dimethyl-3-(1-methylazetidin- 3-yl)-5-oxo-4,5-dihydro-3H- pyrazolo[3,4-c]isoquinolin-9- yl)ethyl)amino)phenyl)-N- methylpicolinamide | 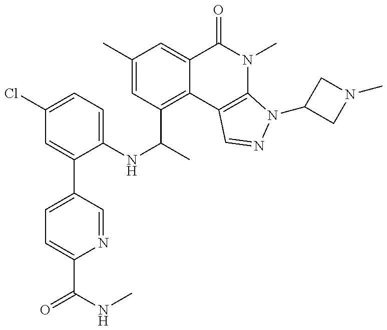 [SMILES: CNC(=O)c1ccc(-c2cc(Cl)ccc2NC(C)c2cc(C)cc3c(=O)n(C)c4c(cnn4C4CN(C)C4)c23)cn1] | 570.2 |
| 87 | 9-(1-((6-chloro-2-(2-methyl-1- oxoisoindolin-5-yl)pyridin-3- yl)amino)ethyl)-4,7-dimethyl- 3-(1-methylazetidin-3-yl)-3,4- dihydro-5H-pyrazolo[3,4- c]isoquinolin-5-one | 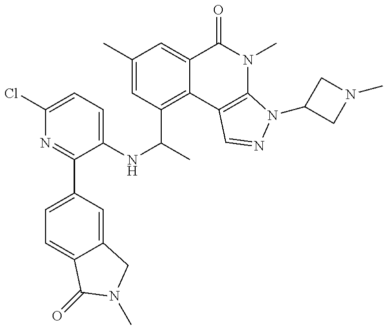 [SMILES: Cc1cc(C(C)Nc2ccc(Cl)nc2-c2ccc3c(c2)CN(C)C3=O)c2c(c1)c(=O)n(C)c1c2cnn1C1CN(C)C1] | 582.2 |
| 88 | 9-(1-((6-chloro-2-(1-methyl- 1H-indazol-5-yl)pyridin-3- yl)amino)ethyl)-4,7-dimethyl- 3-(1-methylazetidin-3-yl)-3,4- dihydro-5H-pyrazolo[3,4- c]isoquinolin-5-one | 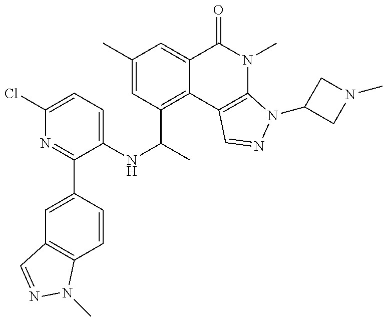 [SMILES: Cc1cc(C(C)Nc2ccc(Cl)nc2-c2ccc3c(cnn3C)c2)c2c(c1)c(=O)n(C)c1c2cnn1C1CN(C)C1] | 567.2 |

### Example 89: 9-(1-((6-chloro-2-(1-methyl-1H-1,2,4-triazol-3-yl)pyridin-3-yl)amino)ethyl)-3-(1-(2-hydroxyethyl) azetidin-3-yl)-4,7-dimethyl-3,4-dihydro-5H-pyrazolo[3,4-c]isoquinolin-5-one

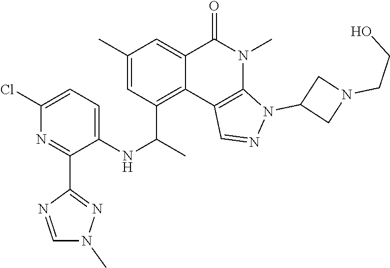 [SMILES: Cc1cc(C(C)Nc2ccc(Cl)nc2-c2ncn(C)n2)c2c(c1)c(=O)n(C)c1c2cnn1C1CN(CCO)C1]

### Step 1: 3-(azetidin-3-yl)-9-(1-((6-chloro-2-(1-methyl-1H-1,2,4-triazol-3-yl)pyridin-3-yl)amino)ethyl)-4,7-dimethyl-3,4-dihydro-5H-pyrazolo[3,4-c]isoquinolin-5-one

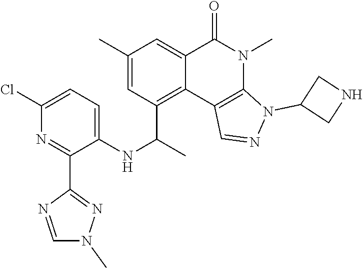 [SMILES: Cc1cc(C(C)Nc2ccc(Cl)nc2-c2ncn(C)n2)c2c(c1)c(=O)n(C)c1c2cnn1C1CNC1]

The title compound was prepared using similar procedures as described for Example 81 with 6-chloro-2-(1-methyl-1H-1,2,4-triazol-3-yl)pyridin-3-amine replacing 6-chloro-2-(1-methyl-1H-pyrazol-4-yl)pyridin-3-amine in Step 10. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₂₇ClN₉O (M+H)⁺ m/z=504.2; found 504.2.

### Step 2: 9-(1-((6-chloro-2-(1-methyl-1H-1,2,4-triazol-3-yl)pyridin-3-yl)amino)ethyl)-3-(1-(2-hydroxyethyl) azetidin-3-yl)-4,7-dimethyl-3,4-dihydro-5H-pyrazolo[3,4-c]isoquinolin-5-one

To a mixture of 3-(azetidin-3-yl)-9-(1-((6-chloro-2-(1-methyl-1H-1,2,4-triazol-3-yl)pyridin-3-yl)amino)ethyl)-4,7-dimethyl-3,4-dihydro-5H-pyrazolo[3,4-c]isoquinolin-5-one (10 mg, 0.02 mmol) was added DCM (0.5 mL), 2-((tert-butyldimethylsilyl)oxy) acetaldehyde (10 mg, 0.05 mmol) and sodium triacetoxyborohydride (5 mg, 0.023 mmol). The mixture was stirred for 30 min, solvent was removed under vacuum. 4 M HCl in dioxane (0.3 mL) and MeOH (0.2 mL) was added and the mixture was stirred for 10 min before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₁ClN₉O₂ (M+H)⁺ m/z=548.2; found 548.2.

### Example 90: 9-(1-((6-chloro-2-(1-methyl-1H-1,2,4-triazol-3-yl)pyridin-3-yl)amino)ethyl)-3-(1-cyclopropylazetidin-3-yl)-4,7-dimethyl-3,4-dihydro-5H-pyrazolo[3,4-c]isoquinolin-5-one

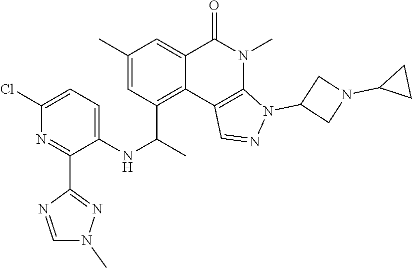 [SMILES: Cc1cc(C(C)Nc2ccc(Cl)nc2-c2ncn(C)n2)c2c(c1)c(=O)n(C)c1c2cnn1C1CN(C2CC2)C1]

To a mixture of 3-(azetidin-3-yl)-9-(1-((6-chloro-2-(1-methyl-1H-1,2,4-triazol-3-yl)pyridin-3-yl)amino)ethyl)-4,7-dimethyl-3,4-dihydro-5H-pyrazolo[3,4-c]isoquinolin-5-one (10 mg, 0.02 mmol) was added MeOH (0.5 mL), (1-ethoxycyclopropoxy)trimethylsilane (30 mg, 0.17 mmol), sodium cyanoborohydride (10 mg, 0.16 mmol) and acetic acid (10 uL). The mixture was stirred for 12 h before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₃₁ClN₉O (M+H)⁺ m/z=544.2; found 544.2.

### Example 91: 9-(1-((6-chloro-2-(1-methyl-1H-1,2,4-triazol-3-yl)pyridin-3-yl)amino)ethyl)-3-(1-((S)-2-hydroxypropyl) azetidin-3-yl)-4,7-dimethyl-3,4-dihydro-5H-pyrazolo[3,4-c]isoquinolin-5-one

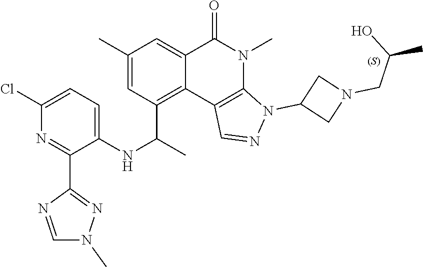

To a mixture of 3-(azetidin-3-yl)-9-(1-((6-chloro-2-(1-methyl-1H-1,2,4-triazol-3-yl)pyridin-3-yl)amino)ethyl)-4,7-dimethyl-3,4-dihydro-5H-pyrazolo[3,4-c]isoquinolin-5-one (10 mg, 0.02 mmol) was added acetonitrile (0.5 mL), (S)-2-methyloxirane (10 mg, 0.17 mmol) and DIEA (10 uL). The mixture was stirred at 70° C. for 6 h before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₃₃ClN₉O₂ (M+H)⁺ m/z=562.2; found 562.2.

### Examples 92-96

Examples 92-96 in Table 9 were prepared similarly as described for Example 91.

TABLE 9

| Example | Chemical | | LCMS |
|---|---|---|---|
| # | name | Structure | m/z (M + H)⁺ |
| 92 | 9-(1-((6-chloro-2-(1- methyl-1H-1,2,4- triazol-3-yl)pyridin-3- yl)amino)ethyl)-3-(1- ((R)-2- hydroxypropyl)azetidin- 3-yl)-4,7-dimethyl- 3,4-dihydro-5H- pyrazolo[3,4- c]isoquinolin-5-one | 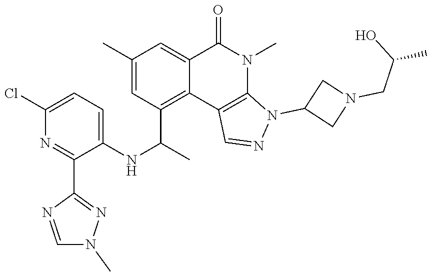 [SMILES: Cc1cc(C(C)Nc2ccc(Cl)nc2-c2ncn(C)n2)c2c(c1)c(=O)n(C)c1c2cnn1C1CN(C[C@@H](C)O)C1] | 562.2 |
| 93 | 9-(1-((6-chloro-2-(1- methyl-1H-1,2,4- triazol-3-yl)pyridin-3- yl)amino)ethyl)-3-(1- (2-hydroxy-2- methylpropyl)azetidin- 3-yl)-4,7-dimethyl- 3,4-dihydro-5H- pyrazolo[3,4- c]isoquinolin-5-one | 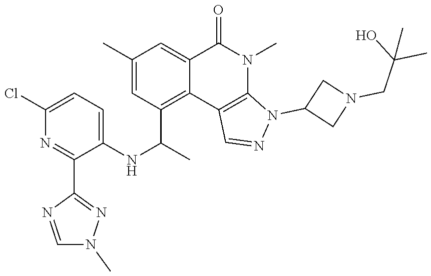 [SMILES: Cc1cc(C(C)Nc2ccc(Cl)nc2-c2ncn(C)n2)c2c(c1)c(=O)n(C)c1c2cnn1C1CN(CC(C)(C)O)C1] | 576.2 |
| 94 | 9-(1-((6-chloro-2-(1- methyl-1H-1,2,4- triazol-3-yl)pyridin-3- yl)amino)ethyl)-3-(1- (2,2- difluoroethyl)azetidin- 3-yl)-4,7-dimethyl- 3,4-dihydro-5H- pyrazolo[3,4- c]isoquinolin-5-one | 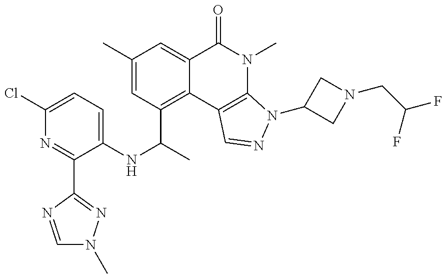 [SMILES: Cc1cc(C(C)Nc2ccc(Cl)nc2-c2ncn(C)n2)c2c(c1)c(=O)n(C)c1c2cnn1C1CN(CC(F)F)C1] | 568.2 |
| 95 | 2-(3-(9-(1-((6-chloro- 2-(1-methyl-1H-1,2,4- triazol-3-yl)pyridin-3- yl)amino)ethyl)-4,7- dimethyl-5-oxo-4,5- dihydro-3H- pyrazolo[3,4- c]isoquinolin-3- yl)azetidin-1- yl)acetonitrile | 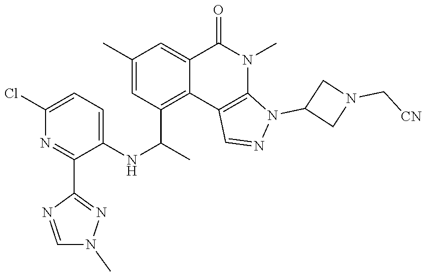 [SMILES: Cc1cc(C(C)Nc2ccc(Cl)nc2-c2ncn(C)n2)c2c(c1)c(=O)n(C)c1c2cnn1C1CN(CC#N)C1] | 543.2 |
| 96 | 3-(3-(9-(1-((6-chloro- 2-(1-methyl-1H-1,2,4- triazol-3-yl)pyridin-3- yl)amino)ethyl)-4,7- dimethyl-5-oxo-4,5- dihydro-3H- pyrazolo[3,4- c]isoquinolin-3- yl)azetidin-1- yl)propanenitrile | 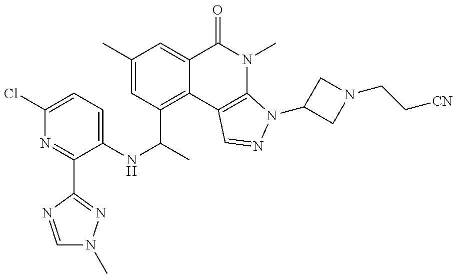 [SMILES: Cc1cc(C(C)Nc2ccc(Cl)nc2-c2ncn(C)n2)c2c(c1)c(=O)n(C)c1c2cnn1C1CN(CCC#N)C1] | 557.2 |

### Example 97: 3-(1-Acetylazetidin-3-yl)-9-(1-((6-chloro-2-(1-methyl-1H-1,2,4-triazol-3-yl)pyridin-3-yl)amino)ethyl)-4,7-dimethyl-3,4-dihydro-5H-pyrazolo[3,4-c]isoquinolin-5-one

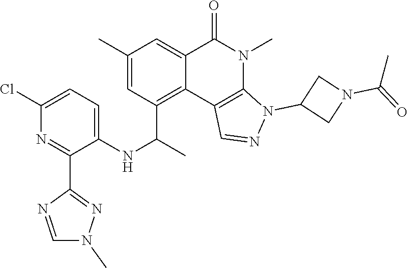 [SMILES: CC(=O)N1CC(n2ncc3c4c(C(C)Nc5ccc(Cl)nc5-c5ncn(C)n5)cc(C)cc4c(=O)n(C)c32)C1]

To a mixture of 3-(azetidin-3-yl)-9-(1-((6-chloro-2-(1-methyl-1H-1,2,4-triazol-3-yl)pyridin-3-yl)amino)ethyl)-4,7-dimethyl-3,4-dihydro-5H-pyrazolo[3,4-c]isoquinolin-5-one (Example 89, step 1) (10 mg, 0.02 mmol) in DCM (0.5 mL) was added acetyl chloride (3.2 mg, 0.04 mmol) DCM (0.2 mL) solution and N,N-diisopropylethylamine (5.0 uL) at 0° C. The reaction mixture was stirred at same temperature for 30 minutes before the solvent was removed under vacuum. The resulting mixture was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₂₉ClN₉O₂ (M+H)⁺ m/z=546.2; found 546.2.

### Examples 98-99

Examples 98-99 in Table 10 were prepared similarly as described for Example 97

TABLE 10

| Example | Chemical | | LCMS |
|---|---|---|---|
| # | Name | Structure | m/z (M + H)⁺ |
| 98 | methyl 3-(9-(1-((6-chloro- 2-(1-methyl-1H-1,2,4- triazol-3-yl)pyridin-3- yl)amino)ethyl)-4,7- dimethyl-5-oxo-4,5- dihydro-3H-pyrazolo[3,4- c]isoquinolin-3- yl)azetidine-1-carboxylate | 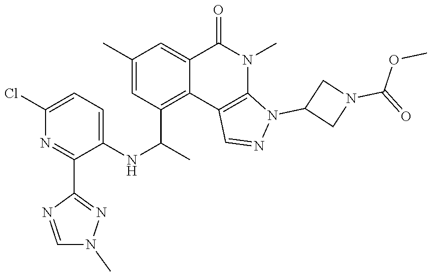 [SMILES: COC(=O)N1CC(n2ncc3c4c(C(C)Nc5ccc(Cl)nc5-c5ncn(C)n5)cc(C)cc4c(=O)n(C)c32)C1] | 562.2 |
| 99 | 9-(1-((6-chloro-2-(1- methyl-1H-1,2,4-triazol-3- yl)pyridin-3- yl)amino)ethyl)-4,7- dimethyl-3-(1- (methylsulfonyl)azetidin- 3-yl)-3,4-dihydro-5H- pyrazolo[3,4- c]isoquinolin-5-one | 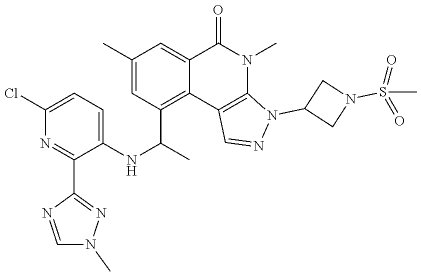 [SMILES: Cc1cc(C(C)Nc2ccc(Cl)nc2-c2ncn(C)n2)c2c(c1)c(=O)n(C)c1c2cnn1C1CN(S(C)(=O)=O)C1] | 582.2 |

### Example 100: 3-(1-Acetylazetidin-3-yl)-9-(1-((6-chloro-1′-methyl-6′-oxo-1′,6′-dihydro-[2,3′-bipyridin]-3-yl)amino)ethyl)-4,7-dimethyl-3,4-dihydro-5H-pyrazolo[3,4-c]isoquinolin-5-one

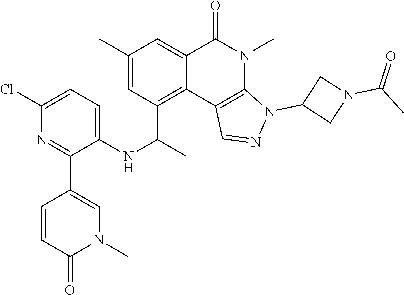 [SMILES: CC(=O)N1CC(n2ncc3c4c(C(C)Nc5ccc(Cl)nc5-c5ccc(=O)n(C)c5)cc(C)cc4c(=O)n(C)c32)C1]

### Step 1: 9-acetyl-3-(azetidin-3-yl)-4,7-dimethyl-3,4-dihydro-5H-pyrazolo[3,4-c]isoquinolin-5-one

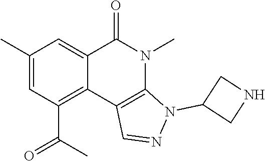 [SMILES: CC(=O)c1cc(C)cc2c(=O)n(C)c3c(cnn3C3CNC3)c12]

To tert-butyl 3-(9-acetyl-4,7-dimethyl-5-oxo-4,5-dihydro-3H-pyrazolo[3,4-c]isoquinolin-3-yl) azetidine-1-carboxylate (Example 81, step 7) (200 mg, 0.57 mmol) in 2 dram vial was added TFA (0.5 mL). The mixture was stirred at rt for 1 h. The solvent was removed under vacuum. The residue was washed with sat. NaHCO₃, then extracted with DCM/IPA 3:1. The combined organic layer was dried over Na₂SO₄ and concentrated under vacuum. The residue was used directly in the next step without further purification. LCMS calculated for C₁₇H₁₉N₄O₂ (M+H)⁺ m/z=311.1; found 311.2.

### Step 2: 9-acetyl-3-(1-acetylazetidin-3-yl)-4,7-dimethyl-3,4-dihydro-5H-pyrazolo[3,4-c]isoquinolin-5-one

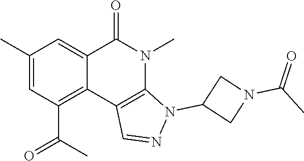 [SMILES: CC(=O)c1cc(C)cc2c(=O)n(C)c3c(cnn3C3CN(C(C)=O)C3)c12]

To a mixture of 9-acetyl-3-(azetidin-3-yl)-4,7-dimethyl-3,4-dihydro-5H-pyrazolo[3,4-c]isoquinolin-5-one (110 mg, 0.37 mmol) in DCM (3 mL) was added acetyl chloride (32 mg, 0.4 mmol) DCM (2 mL) solution and N,N-diisopropylethylamine (110 uL 0.6 mmol) at 0° C. The reaction mixture was stirred at same temperature for 30 minutes before the solvent was removed under vacuum. The resulting mixture was as washed with sat. NaHCO₃, then extracted with EtOAc. The combined organic layer was dried over Na₂SO₄ and concentrated under vacuum. The residue was used directly in the next step without further purification. LCMS calculated for C₁₉H₂₁N₄O₃ (M+H)⁺ m/z=353.2; found 353.2.

### Step 3: 3-(1-acetylazetidin-3-yl)-9-(1-hydroxyethyl)-4,7-dimethyl-3,4-dihydro-5H-pyrazolo[3,4-c]isoquinolin-5-one

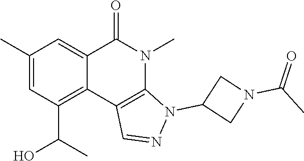 [SMILES: CC(=O)N1CC(n2ncc3c4c(C(C)O)cc(C)cc4c(=O)n(C)c32)C1]

To a mixture of 9-acetyl-3-(1-acetylazetidin-3-yl)-4,7-dimethyl-3,4-dihydro-5H-pyrazolo[3,4-c]isoquinolin-5-one (80 mg, 0.23 mmol) in MeOH (1 mL) and DCM (1 mL) was added NaBH₄ (12 mg, 0.3 mmol) at 0° C. The resulting mixture was stirred at same temperature for 10 min before quenched with sat. NH₄Cl. The mixture was diluted with water and extracted with DCM/IPA 3:1. The combined organics were washed with sat. NaCl and dried with anhydrous sodium sulfate, filtered, and concentrated under reduced pressure. The residue was purified by silica gel column chromatography, eluted with DCM and MeOH to provide the desired product as a white solid (73 mg, 90%). LCMS calculated for C₁₉H₂₃N₄O₃ (M+H)⁺ m/z=355.2; found 355.2.

### Step 4: 3-(1-acetylazetidin-3-yl)-9-(1-bromoethyl)-4,7-dimethyl-3,4-dihydro-5H-pyrazolo[3,4-c]isoquinolin-5-one

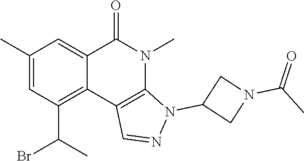 [SMILES: CC(=O)N1CC(n2ncc3c4c(C(C)Br)cc(C)cc4c(=O)n(C)c32)C1]

To a mixture of 3-(1-acetylazetidin-3-yl)-9-(1-hydroxyethyl)-4,7-dimethyl-3,4-dihydro-5H-pyrazolo[3,4-c]isoquinolin-5-one (73 mg, 0.21 mmol) in THF (1 mL) was added pyridine (29 uL, 0.36 mmol) PBr₃ (65 mg, 0.24 mmol). The resulting mixture was stirred at rt for 2 h. The mixture was then quenched with sat. NaHCO₃ and extracted with EtOAc. The combined organics were washed with sat. NaCl, dried with anhydrous sodium sulfate, filtered, and concentrated under reduced pressure. The residue was used directly in the next step without further purification.

### Step 5: 3-(1-acetylazetidin-3-yl)-9-(1-((6-chloro-1′-methyl-6′-oxo-1′,6′-dihydro-[2,3′-bipyridin]-3-yl)amino)ethyl)-4,7-dimethyl-3,4-dihydro-5H-pyrazolo[3,4-c]isoquinolin-5-one

3-(1-Acetylazetidin-3-yl)-9-(1-bromoethyl)-4,7-dimethyl-3,4-dihydro-5H-pyrazolo[3,4-c]isoquinolin-5-one (10 mg, 0.024 mmol) in 2 dram vial was added DMF (0.2 mL), and 3-amino-3-amino-6-chloro-1′-methyl-[2,3′-bipyridin]-6′(1′H)-one (intermediate 24) (10 mg, 0.04 mmol). The mixture was heated to 80° C. for 1 h, then diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₀H₃₁ClN₇O₃ (M+H)⁺ m/z=572.2; found 572.2.

### Examples 101-102

Examples 101-102 in Table 11 were prepared similarly as described for Example 100.

TABLE 11

| Example | Chemical | | LCMS |
|---|---|---|---|
| # | name | Structure | m/z (M + H)⁺ |
| 101 | 3-((1-(3-(1-acetylazetidin-3- yl)-4,7-dimethyl-5-oxo-4,5- dihydro-3H-pyrazolo[3,4- c]isoquinolin-9- yl)ethyl)amino)-6-chloro-N- methyl-[2,3′-bipyridine]-6′- carboxamide | 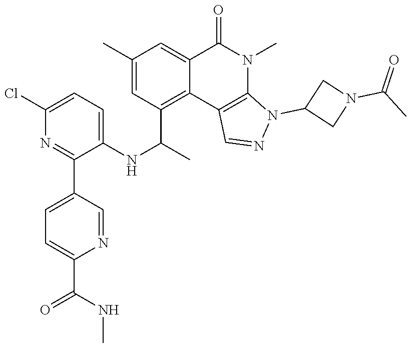 [SMILES: CNC(=O)c1ccc(-c2nc(Cl)ccc2NC(C)c2cc(C)cc3c(=O)n(C)c4c(cnn4C4CN(C(C)=O)C4)c23)cn1] | 599.2 |
| 102 | 3-(1-acetylazetidin-3-yl)-9-(1- ((6-chloro-2-(1-(2- hydroxyethyl)-1H-1,2,4- triazol-3-yl)pyridin-3- yl)amino)ethyl)-4,7-dimethyl- 3,4-dihydro-5H-pyrazolo[3,4- c]isoquinolin-5-one | 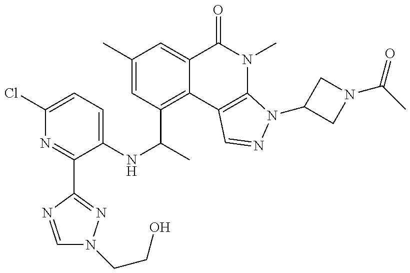 [SMILES: CC(=O)N1CC(n2ncc3c4c(C(C)Nc5ccc(Cl)nc5-c5ncn(CCO)n5)cc(C)cc4c(=O)n(C)c32)C1] | 576.2 |

### Example 103: 9-(1-((6-Chloro-2-(1-methyl-1H-1,2,4-triazol-3-yl)pyridin-3-yl)amino)ethyl)-3-(1-(2-hydroxyacetyl) azetidin-3-yl)-4,7-dimethyl-3,4-dihydro-5H-pyrazolo[3,4-c]isoquinolin-5-one

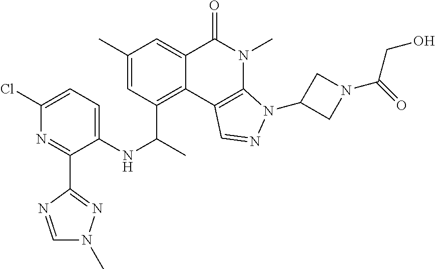 [SMILES: Cc1cc(C(C)Nc2ccc(Cl)nc2-c2ncn(C)n2)c2c(c1)c(=O)n(C)c1c2cnn1C1CN(C(=O)CO)C1]

To a mixture of 3-(azetidin-3-yl)-9-(1-((6-chloro-2-(1-methyl-1H-1,2,4-triazol-3-yl)pyridin-3-yl)amino)ethyl)-4,7-dimethyl-3,4-dihydro-5H-pyrazolo[3,4-c]isoquinolin-5-one (Example 89, step 1) (10 mg, 0.02 mmol) was added DMF (0.5 mL), 2-hydroxyacetic acid (10 mg, 0.13 mmol), HATU (10 mg, 0.026 mmol) and DIEA (10 uL) at 0° C. The mixture was warmed to rt and stirred for 2 h before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₂₉ClN₉O₃ (M+H)⁺ m/z=562.2; found 562.2.

### Example 104: 9-(1-((6-Chloro-2-(1-methyl-1H-1,2,4-triazol-3-yl)pyridin-3-yl)amino)ethyl)-3-(1-(dimethylglycyl) azetidin-3-yl)-4,7-dimethyl-3,4-dihydro-5H-pyrazolo[3,4-c]isoquinolin-5-one

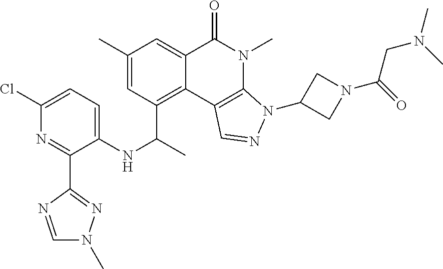 [SMILES: Cc1cc(C(C)Nc2ccc(Cl)nc2-c2ncn(C)n2)c2c(c1)c(=O)n(C)c1c2cnn1C1CN(C(=O)CN(C)C)C1]

The title compound was prepared using similar procedures as described for Example 103 with dimethylglycine replacing 2-hydroxyacetic acid. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₄ClN₁₀O₂ (M+H)⁺ m/z=589.2; found 589.2.

### Example 105: 9-(1-((6-Chloro-2-(1-methyl-1H-1,2,4-triazol-3-yl)pyridin-3-yl)amino)ethyl)-7-methyl-4-(methyl-d3)-3-((S)-1-methylpyrrolidin-3-yl)-3,4-dihydro-5H-pyrazolo[3,4-c]isoquinolin-5-one

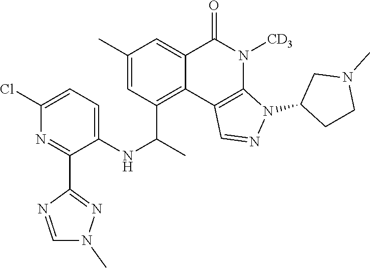 [SMILES: [2H]C([2H])([2H])n1c(=O)c2cc(C)cc(C(C)Nc3ccc(Cl)nc3-c3ncn(C)n3)c2c2cnn([C@H]3CCN(C)C3)c21]

The title compound was prepared using similar procedures as described for Example 81 with tert-butyl (R)-3-((methylsulfonyl)oxy) pyrrolidine-1-carboxylate replacing tert-butyl 3-((methylsulfonyl)oxy) azetidine-1-carboxylate in step 1, CD₃I replacing methyl iodide in step 5, and 6-chloro-2-(1-methyl-1H-1,2,4-triazol-3-yl)pyridin-3-amine replacing 6-chloro-2-(1-methyl-1H-pyrazol-4-yl)pyridin-3-amine in step 10. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₂₈D₃ClN₉O (M+H)⁺ m/z=535.3; found 535.2.

### Example 106: 9-(1-((6-Chloro-2-(1-methyl-1H-pyrazol-4-yl)pyridin-3-yl)amino)ethyl)-3-((1-(2,2-difluoroethyl)piperidin-4-yl)methyl)-4,7-dimethyl-3,4-dihydro-5H-pyrazolo[3,4-c]isoquinolin-5-one

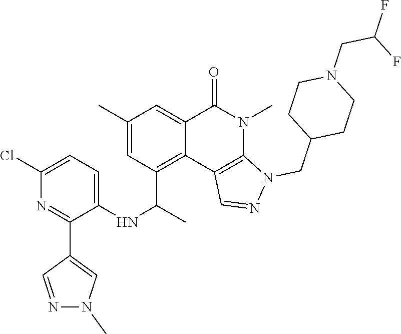 [SMILES: Cc1cc(C(C)Nc2ccc(Cl)nc2-c2cnn(C)c2)c2c(c1)c(=O)n(C)c1c2cnn1CC1CCN(CC(F)F)CC1]

### Step 1: benzyl 4-((9-(1-bromoethyl)-4,7-dimethyl-5-oxo-4,5-dihydro-3H-pyrazolo[3,4-c]isoquinolin-3-yl)methyl)piperidine-1-carboxylate

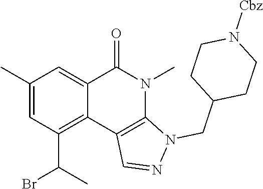 [SMILES: Cc1cc(C(C)Br)c2c(c1)c(=O)n(C)c1c2cnn1CC1CCN(C(=O)OCc2ccccc2)CC1]

The title compound was prepared using similar procedures as described for Example 81 with benzyl 4-(hydrazineylmethyl)piperidine-1-carboxylate replacing tert-butyl 3-hydrazineylazetidine-1-carboxylate in step 2.

### Step 2: benzyl 4-((9-(1-((6-chloro-2-(1-methyl-1H-pyrazol-4-yl)pyridin-3-yl)amino)ethyl)-4,7-dimethyl-5-oxo-4,5-dihydro-3H-pyrazolo[3,4-c]isoquinolin-3-yl)methyl)piperidine-1-carboxylate

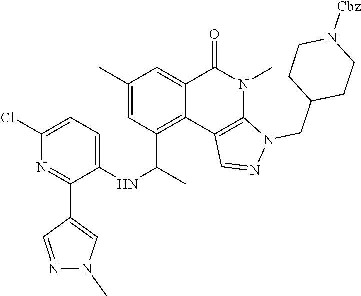 [SMILES: Cc1cc(C(C)Nc2ccc(Cl)nc2-c2cnn(C)c2)c2c(c1)c(=O)n(C)c1c2cnn1CC1CCN(C(=O)OCc2ccccc2)CC1]

To benzyl 4-((9-(1-bromoethyl)-4,7-dimethyl-5-oxo-4,5-dihydro-3H-pyrazolo[3,4-c]isoquinolin-3-yl)methyl)piperidine-1-carboxylate (100 mg, 0.18 mmol) in 2 dram vial was added DMF (0.5 mL), and 6-chloro-2-(1-methyl-1H-pyrazol-4-yl)pyridin-3-amine (100 mg, 0.5 mmol). The mixture was heated at 80° C. for 1 h. Upon cooling to room temperature and diluted with water. The mixture was extracted with EtOAc, the combined organic layer was dried over Na₂SO₄ and concentrated under vacuum. The residue was purified by silica gel column chromatography, eluted with DCM and EtOAc to provide the desired product as a brown solid (73 mg, 60%). LCMS calculated for C₃₇H₄₀ClN₈O₃ (M+H)⁺ m/z=679.3; found 679.3.

### Step 3: 9-(1-((6-chloro-2-(1-methyl-1H-pyrazol-4-yl)pyridin-3-yl)amino)ethyl)-4,7-dimethyl-3-(piperidin-4-ylmethyl)-3,4-dihydro-5H-pyrazolo[3,4-c]isoquinolin-5-one

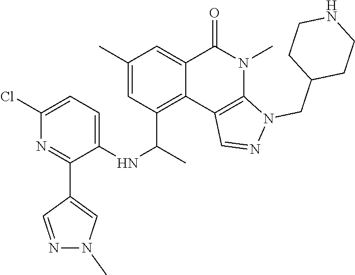 [SMILES: Cc1cc(C(C)Nc2ccc(Cl)nc2-c2cnn(C)c2)c2c(c1)c(=O)n(C)c1c2cnn1CC1CCNCC1]

To a mixture of benzyl 4-((9-(1-((6-chloro-2-(1-methyl-1H-pyrazol-4-yl)pyridin-3-yl)amino)ethyl)-4,7-dimethyl-5-oxo-4,5-dihydro-3H-pyrazolo[3,4-c]isoquinolin-3-yl)methyl)piperidine-1-carboxylate (73 mg, 0.11 mmol) was added Pd/C (10 wt %, 20 mg) and MeOH (4 mL). The reaction mixture was purged with hydrogen, stirred at rt for 2 h. After completion, the mixture was filtered. The solvent was then removed under vacuum to afford the crude product as a pale oil, which was used directly without further purification. LCMS calculated for C₂₉H₃₄ClN₈O (M+H)⁺ m/z=545.3; found 545.3.

### Step 4: 9-(1-((6-chloro-2-(1-methyl-1H-pyrazol-4-yl)pyridin-3-yl)amino)ethyl)-3-((1-(2,2-difluoroethyl)piperidin-4-yl)methyl)-4,7-dimethyl-3,4-dihydro-5H-pyrazolo[3,4-c]isoquinolin-5-one

To a mixture of 9-(1-((6-chloro-2-(1-methyl-1H-pyrazol-4-yl)pyridin-3-yl)amino)ethyl)-4,7-dimethyl-3-(piperidin-4-ylmethyl)-3,4-dihydro-5H-pyrazolo[3,4-c]isoquinolin-5-one (10 mg, 0.018 mmol), DIEA (10 uL) and MeCN (0.5 mL) was added 2,2-difluoroethyl trifluoromethanesulfonate (10 uL). The reaction mixture was heated and stirred at 60° C. for 1 h. After completion, the resulting mixture was dilute with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₁H₃₆ClF₂N₈O (M+H)⁺ m/z=609.3; found 609.2.

### Examples 107-108

Examples 107-108 in Table 12 were prepared similarly as described for Example 106.

TABLE 12

| Example | Chemical | | LCMS |
|---|---|---|---|
| # | Name | Structure | m/z (M + H)⁺ |
| 107 | 3-((1-acetylpiperidin-4- yl)methyl)-9-(1-((6-chloro-2- (1-methyl-1H-pyrazol-4- yl)pyridin-3-yl)amino)ethyl)- 4,7-dimethyl-3,4-dihydro-5H- pyrazolo[3,4-c]isoquinolin-5- one | 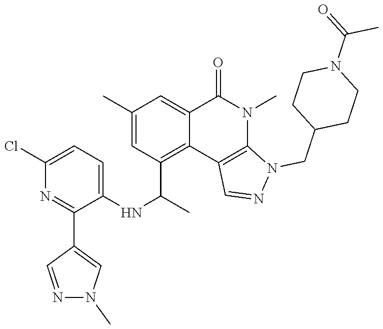 [SMILES: CC(=O)N1CCC(Cn2ncc3c4c(C(C)Nc5ccc(Cl)nc5-c5cnn(C)c5)cc(C)cc4c(=O)n(C)c32)CC1] | 587.3 |
| 108 | 4-((9-(1-((6-chloro-2-(1- methyl-1H-pyrazol-4- yl)pyridin-3-yl)amino)ethyl)- 4,7-dimethyl-5-oxo-4,5- dihydro-3H-pyrazolo[3,4- c]isoquinolin-3-yl)methyl)- N,N-dimethylpiperidine-1- carboxamide | 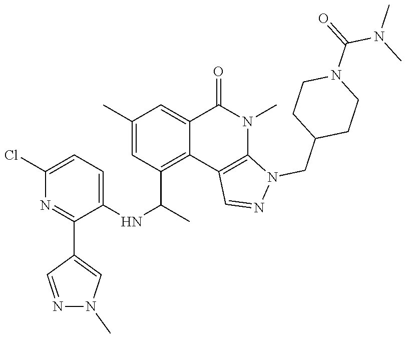 [SMILES: Cc1cc(C(C)Nc2ccc(Cl)nc2-c2cnn(C)c2)c2c(c1)c(=O)n(C)c1c2cnn1CC1CCN(C(=O)N(C)C)CC1] | 616.3 |

### Example 109: 4-((9-(1-((6-Chloro-3′-fluoro-[2,4′-bipyridin]-3-yl)amino)ethyl)-4,7-dimethyl-5-oxo-4,5-dihydro-3H-pyrazolo[3,4-c]isoquinolin-3-yl)methyl)-N-ethylpiperidine-1-carboxamide

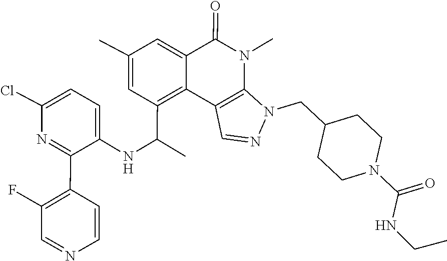 [SMILES: CCNC(=O)N1CCC(Cn2ncc3c4c(C(C)Nc5ccc(Cl)nc5-c5ccncc5F)cc(C)cc4c(=O)n(C)c32)CC1]

### Step 1: 9-(1-((6-chloro-3′-fluoro-[2,4′-bipyridin]-3-yl)amino)ethyl)-4,7-dimethyl-3-(piperidin-4-ylmethyl)-3,4-dihydro-5H-pyrazolo[3,4-c]isoquinolin-5-one

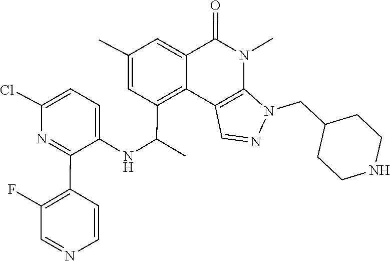 [SMILES: Cc1cc(C(C)Nc2ccc(Cl)nc2-c2ccncc2F)c2c(c1)c(=O)n(C)c1c2cnn1CC1CCNCC1]

The title compound was prepared using similar procedures as described for Example 106 with 6-chloro-3′-fluoro-[2,4′-bipyridin]-3-amine replacing 6-chloro-2-(1-methyl-1H-pyrazol-4-yl)pyridin-3-amine in step 2. LCMS calculated for C₃₀H₃₂ClFN₇O (M+H)⁺ m/z=560.2; found 560.2.

### Step 2: 4-((9-(1-((6-chloro-3′-fluoro-[2,4′-bipyridin]-3-yl)amino)ethyl)-4,7-dimethyl-5-oxo-4,5-dihydro-3H-pyrazolo[3,4-c]isoquinolin-3-yl)methyl)-N-ethylpiperidine-1-carboxamide

To a mixture of 9-(1-((6-chloro-3′-fluoro-[2,4′-bipyridin]-3-yl)amino)ethyl)-4,7-dimethyl-3-(piperidin-4-ylmethyl)-3,4-dihydro-5H-pyrazolo[3,4-c]isoquinolin-5-one (10 mg, 0.018 mmol), and MeCN (0.5 mL) was added ethyl isocyanate (10 uL). The reaction mixture was heated and stirred at 80° C. for 1 h. After completion, the resulting mixture was dilute with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₃H₃₇ClFN₈O₂ (M+H)⁺ m/z=631.3; found 631.2.

### Example 110: 9-(1-((6-Chloro-3′-fluoro-[2,4′-bipyridin]-3-yl)amino)ethyl)-3-((1-(2,2-difluoroethyl) pyrrolidin-3-yl)methyl)-4,7-dimethyl-3,4-dihydro-5H-pyrazolo[3,4-c]isoquinolin-5-one

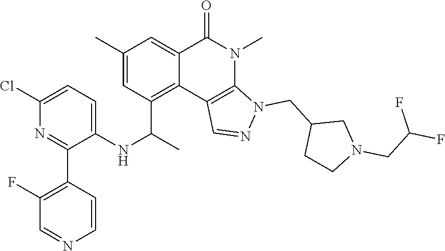 [SMILES: Cc1cc(C(C)Nc2ccc(Cl)nc2-c2ccncc2F)c2c(c1)c(=O)n(C)c1c2cnn1CC1CCN(CC(F)F)C1]

The titled compound was prepared using similar procedures as described for Example 106 with benzyl 3-(hydrazineylmethyl) pyrrolidine-1-carboxylate replacing 4-(hydrazineylmethyl)piperidine-1-carboxylate in step 1 and 6-chloro-3′-fluoro-[2,4′-bipyridin]-3-amine replacing 6-chloro-2-(1-methyl-1H-pyrazol-4-yl)pyridin-3-amine in step 2. The product was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₁H₃₂ClF₃N₇O (M+H)⁺ m/z=610.2; found 610.2.

### Example 111: 9-(1-((6-Chloro-2-(1-methyl-1H-1,2,4-triazol-3-yl)pyridin-3-yl)amino)ethyl)-3-(1-hydroxyethyl)-4,7-dimethylimidazo[1,5-a]quinazolin-5(4H)-one

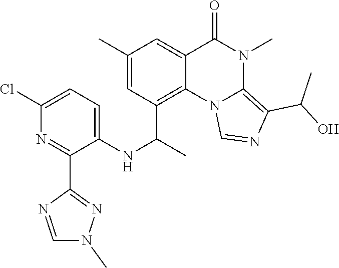 [SMILES: Cc1cc(C(C)Nc2ccc(Cl)nc2-c2ncn(C)n2)c2c(c1)c(=O)n(C)c1c(C(C)O)ncn21]

### Step 1: 9-(1-((6-chloro-2-(1-methyl-1H-1,2,4-triazol-3-yl)pyridin-3-yl)amino)ethyl)-4,7-dimethyl-5-oxo-4,5-dihydroimidazo[1,5-a]quinazoline-3-carboxylic acid

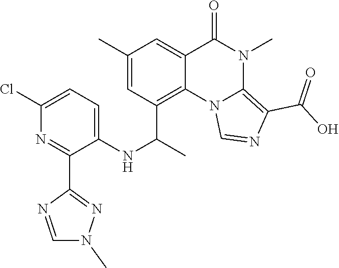 [SMILES: Cc1cc(C(C)Nc2ccc(Cl)nc2-c2ncn(C)n2)c2c(c1)c(=O)n(C)c1c(C(=O)O)ncn21]

The title compound was prepared using similar procedures as described for Example 4 with 6-chloro-2-(1-methyl-1H-1,2,4-triazol-3-yl)pyridin-3-amine replacing methyl 2-aminobenzoate in step 10. The crude product was used directly without further purification. LCMS calculated for C₂₃H₂₂ClN₈O₃ (M+H)⁺ m/z=493.1; found 493.1.

### Step 2: 9-(1-((6-chloro-2-(1-methyl-1H-1,2,4-triazol-3-yl)pyridin-3-yl)amino)ethyl)-3-iodo-4,7-dimethylimidazo[1,5-a]quinazolin-5(4H)-one

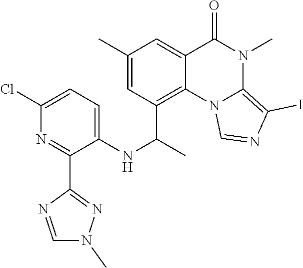 [SMILES: Cc1cc(C(C)Nc2ccc(Cl)nc2-c2ncn(C)n2)c2c(c1)c(=O)n(C)c1c(I)ncn21]

To a mixture of 9-(1-((6-chloro-2-(1-methyl-1H-1,2,4-triazol-3-yl)pyridin-3-yl)amino)ethyl)-4,7-dimethyl-5-oxo-4,5-dihydroimidazo[1,5-a]quinazoline-3-carboxylic acid (520 mg, 1.05 mmol) in DMF (5 mL) under nitrogen was added NaHCO₃ (441 mg, 5.25 mmol) at 0° C., after stirring for 5 min, NIS (268 mg, 1.2 mmol) was added in one portion. The resulting mixture was warmed to rt and stirred for 2 h under vacuum. After completion, the reaction mixture was quenched with sat. Na₂S₂O₃ and diluted with water. The crude product was collected as a brown solid by filtration which was purified by silica gel column chromatography, eluted with DCM and EtOAc to provide the desired product as a brown solid (488 mg, 81%). LCMS calculated for C₂₂H₂₁ClIN₈O (M+H)⁺ m/z=575.1; found 575.1.

### Step 3: 3-acetyl-9-(1-((6-chloro-2-(1-methyl-1H-1,2,4-triazol-3-yl)pyridin-3-yl)amino)ethyl)-4,7-dimethylimidazo[1,5-a]quinazolin-5(4H)-one

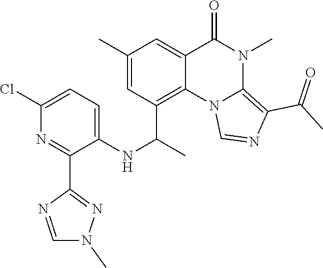 [SMILES: CC(=O)c1ncn2c3c(C(C)Nc4ccc(Cl)nc4-c4ncn(C)n4)cc(C)cc3c(=O)n(C)c12]

The mixture of 9-(1-((6-chloro-2-(1-methyl-1H-1,2,4-triazol-3-yl)pyridin-3-yl)amino)ethyl)-3-iodo-4,7-dimethylimidazo[1,5-a]quinazolin-5(4H)-one (50 mg, 0.087 mmol), PdCl₂(PPh₃)₂ (7 mg, 0.01 mmol), and tributyl(1-ethoxyvinyl)stannane (32 mg, 0.09 mmol) in dioxane (1 mL) was heated at 100° C. for 4 h under nitrogen atmosphere. Upon cooling to room temperature, the mixture was added 2 N HCl and stirred for 30 min before the mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and MeOH to provide the desired product as a brown solid (32 mg, 76%). LCMS calculated for C₂₄H₂₄ClN₈O₂ (M+H)⁺ m/z=491.2; found 491.2.

### Step 4: 9-(1-((6-chloro-2-(1-methyl-1H-1,2,4-triazol-3-yl)pyridin-3-yl)amino)ethyl)-3-(1-hydroxyethyl)-4,7-dimethylimidazo[1,5-a]quinazolin-5(4H)-one

To a mixture of 3-acetyl-9-(1-((6-chloro-2-(1-methyl-1H-1,2,4-triazol-3-yl)pyridin-3-yl)amino)ethyl)-4,7-dimethylimidazo[1,5-a]quinazolin-5(4H)-one (10 mg, 0.02 mmol), and MeOH (0.5 mL) was added NaBH₄ (5 mg, 0.12 mmol) at 0° C. The reaction mixture was stirred at same temperature for 10 min before quenched with sat. NH₄Cl. The resulting mixture was dilute with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₆ClN₈O₂ (M+H)⁺ m/z=493.2; found 493.2.

### Example 112: 9-(1-((6-Chloro-2-(1-methyl-1H-1,2,4-triazol-3-yl)pyridin-3-yl)amino)ethyl)-4,7-dimethyl-3-(pyridin-3-yl) imidazo[1,5-a]quinazolin-5(4H)-one

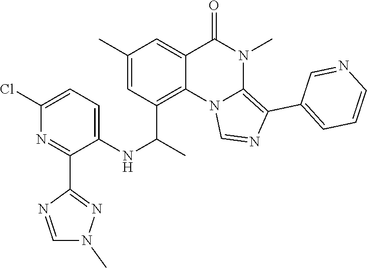 [SMILES: Cc1cc(C(C)Nc2ccc(Cl)nc2-c2ncn(C)n2)c2c(c1)c(=O)n(C)c1c(-c3cccnc3)ncn21]

The mixture of 9-(1-((6-chloro-2-(1-methyl-1H-1,2,4-triazol-3-yl)pyridin-3-yl)amino)ethyl)-3-iodo-4,7-dimethylimidazo[1,5-a]quinazolin-5(4H)-one (Example 111, step 2) (10 mg, 0.02 mmol), pyridin-3-ylboronic acid (5 mg, 0.04 moml), K₃PO₄ (10 mg, 0.05 mmol) and Pd(PPh₃)₄ (2 mg, 0.002 mmol) in dioxane (0.5 mL) and water (0.1 mL) was heated at 100° C. for 20 min under nitrogen atmosphere. The mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₂₅ClN₉O (M+H)⁺ m/z=526.2; found 526.2.

### Examples 113-115

Examples 113-115 in Table 13 were prepared similarly as described for Example 112.

TABLE 13

| Example | Chemical | | LCMS |
|---|---|---|---|
| # | name | Structure | m/z (M + H)⁺ |
| 113 | 9-(1-((6-chloro-2-(1-methyl-1H- 1,2,4-triazol-3-yl)pyridin-3- yl)amino)ethyl)-4,7-dimethyl-3- (2-methylpyridin-3- yl)imidazo[1,5-a]quinazolin- 5(4H)-one | 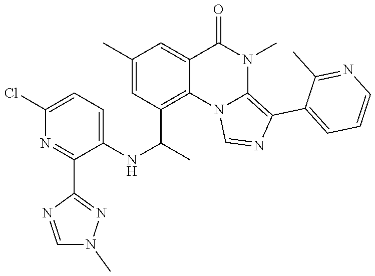 [SMILES: Cc1cc(C(C)Nc2ccc(Cl)nc2-c2ncn(C)n2)c2c(c1)c(=O)n(C)c1c(-c3cccnc3C)ncn21] | 540.2 |
| 114 | 9-(1-((6-chloro-2-(1-methyl-1H- 1,2,4-triazol-3-yl)pyridin-3- yl)amino)ethyl)-4,7-dimethyl-3- (pyrimidin-5-yl)imidazo[1,5- a]quinazolin-5(4H)-one | 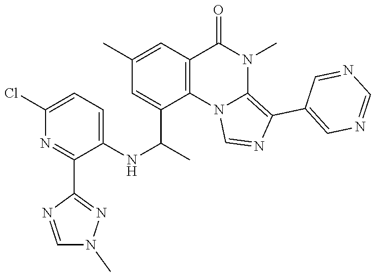 [SMILES: Cc1cc(C(C)Nc2ccc(Cl)nc2-c2ncn(C)n2)c2c(c1)c(=O)n(C)c1c(-c3cncnc3)ncn21] | 527.2 |
| 115 | 9-(1-((6-chloro-2-(1-methyl-1H- 1,2,4-triazol-3-yl)pyridin-3- yl)amino)ethyl)-4,7-dimethyl-3- (2-methylpyridin-4- yl)imidazo[1,5-a]quinazolin- 5(4H)-one | 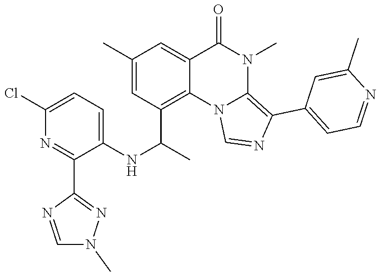 [SMILES: Cc1cc(C(C)Nc2ccc(Cl)nc2-c2ncn(C)n2)c2c(c1)c(=O)n(C)c1c(-c3ccnc(C)c3)ncn21] | 540.2 |

### Example 116: 9-(1-((6-Chloro-2-(1-methyl-1H-pyrazol-4-yl)pyridin-3-yl)amino)ethyl)-4,7-dimethyl-3-(2-methylpyridin-3-yl) imidazo[1,5-a]quinazolin-5(4H)-one

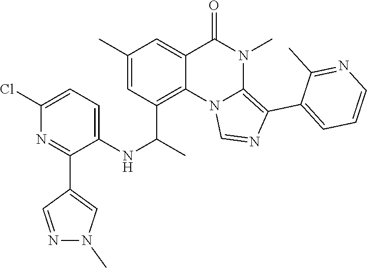 [SMILES: Cc1cc(C(C)Nc2ccc(Cl)nc2-c2cnn(C)c2)c2c(c1)c(=O)n(C)c1c(-c3cccnc3C)ncn21]

### Step 1: 9-(1-((6-chloro-2-(1-methyl-1H-pyrazol-4-yl)pyridin-3-yl)amino)ethyl)-4,7-dimethyl-5-oxo-4,5-dihydroimidazo[1,5-a]quinazoline-3-carboxylic acid

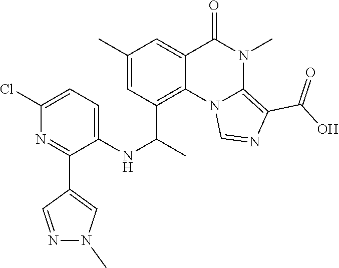 [SMILES: Cc1cc(C(C)Nc2ccc(Cl)nc2-c2cnn(C)c2)c2c(c1)c(=O)n(C)c1c(C(=O)O)ncn21]

The title compound was prepared using similar procedures as described for Example 4 with 6-chloro-2-(1-methyl-1H-pyrazol-4-yl)pyridin-3-amine (intermediate 18) replacing methyl 2-aminobenzoate in step 10. The crude product was used directly without further purification. LCMS calculated for C₂₄H₂₃ClN₇O₃ (M+H)⁺ m/z=492.2; found 492.2.

### Step 2: 9-(1-((6-chloro-2-(1-methyl-1H-pyrazol-4-yl)pyridin-3-yl)amino)ethyl)-3-iodo-4,7-dimethylimidazo[1,5-a]quinazolin-5(4H)-one

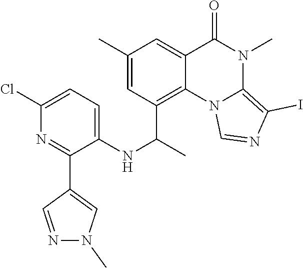 [SMILES: Cc1cc(C(C)Nc2ccc(Cl)nc2-c2cnn(C)c2)c2c(c1)c(=O)n(C)c1c(I)ncn21]

To a mixture of 9-(1-((6-chloro-2-(1-methyl-1H-pyrazol-4-yl)pyridin-3-yl)amino)ethyl)-4,7-dimethyl-5-oxo-4,5-dihydroimidazo[1,5-a]quinazoline-3-carboxylic acid (260 mg, 0.5 mmol) in DMF (2 mL) under nitrogen was added NaHCO₃ (220 mg, 2.6 mmol) at 0° C., after stirring for 5 min, NIS (134 mg, 0.6 mmol) was added in one portion. The resulting mixture was warmed to rt and stirred for 2 h under vacuum. After completion, the reaction mixture was quenched with sat. Na₂S₂O₃ and diluted with water. The crude product was collected as a brown solid by filtration which was purified by silica gel column chromatography, eluted with DCM and EtOAc to provide the desired product as a brown solid (230 mg, 75%). LCMS calculated for C₂₃H₂₂ClIN₇O (M+H)⁺ m/z=574.1; found 574.1.

### Step 3: 9-(1-((6-chloro-2-(1-methyl-1H-pyrazol-4-yl)pyridin-3-yl)amino)ethyl)-4,7-dimethyl-3-(2-methylpyridin-3-yl) imidazo[1,5-a]quinazolin-5(4H)-one

The mixture of 9-(1-((6-chloro-2-(1-methyl-1H-pyrazol-4-yl)pyridin-3-yl)amino)ethyl)-3-iodo-4,7-dimethylimidazo[1,5-a]quinazolin-5(4H)-one (10 mg, 0.02 mmol), 2-methyl-3-(4,4,5,5-tetramethyl-1,3,2-dioxaborolan-2-yl)pyridine (5 mg, 0.04 mol), K₃PO₄ (10 mg, 0.05 mmol) and Pd(PPh₃)₄ (2 mg, 0.002 mmol) in dioxane (0.5 mL) and water (0.1 mL) was heated at 100° C. for 20 min under nitrogen atmosphere. The mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₂₈ClN₈O (M+H)⁺ m/z=539.2; found 539.2.

### Examples 117-118

Examples 117-118 in Table 14 were prepared similarly as described for Example 116.

TABLE 14

| Example | Chemical | | LCMS |
|---|---|---|---|
| # | name | Structure | m/z (M + H)⁺ |
| 117 | 9-(1-((6-chloro-2-(1-methyl-1H- pyrazol-4-yl)pyridin-3- yl)amino)ethyl)-4,7-dimethyl-3- (pyrimidin-5-yl)imidazo[1,5- a]quinazolin-5(4H)-one | 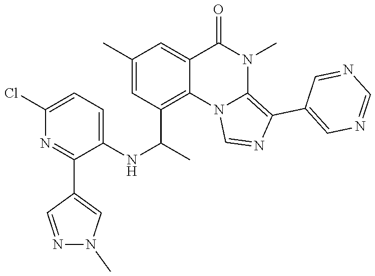 [SMILES: Cc1cc(C(C)Nc2ccc(Cl)nc2-c2cnn(C)c2)c2c(c1)c(=O)n(C)c1c(-c3cncnc3)ncn21] | 526.2 |
| 118 | 4-(9-(1-((6-chloro-2-(1-methyl- 1H-pyrazol-4-yl)pyridin-3- yl)amino)ethyl)-4,7-dimethyl-5- oxo-4,5-dihydroimidazo[1,5- a]quinazolin-3-yl)-N- methylpicolinamide | 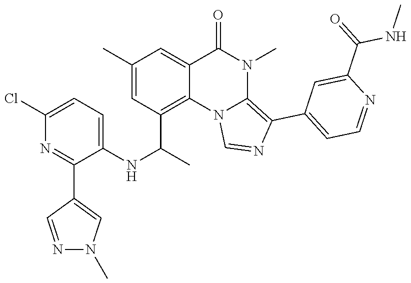 [SMILES: CNC(=O)c1cc(-c2ncn3c4c(C(C)Nc5ccc(Cl)nc5-c5cnn(C)c5)cc(C)cc4c(=O)n(C)c23)ccn1] | 582.2 |

### Example 119: 9-(1-((6-Chloro-2-(1-methyl-1H-1,2,4-triazol-3-yl)pyridin-3-yl)amino)ethyl)-N,N,4,7-tetramethyl-5-oxo-4,5-dihydroimidazo[1,5-a]quinazoline-3-carboxamide

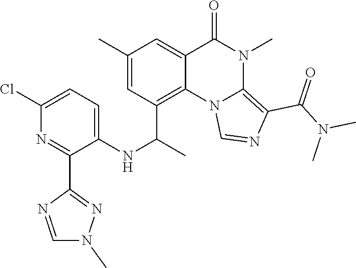 [SMILES: Cc1cc(C(C)Nc2ccc(Cl)nc2-c2ncn(C)n2)c2c(c1)c(=O)n(C)c1c(C(=O)N(C)C)ncn21]

To a solution of 9-(1-((6-chloro-2-(1-methyl-1H-1,2,4-triazol-3-yl)pyridin-3-yl)amino)ethyl)-4,7-dimethyl-5-oxo-4,5-dihydroimidazo[1,5-a]quinazoline-3-carboxylic acid (Example 111, step 1) (20 mg, 0.04 mmol) in DMF (0.5 ml) was added propanephosphonic acid anhydride (50% THF solution, 32 μL, 0.05 mmol) and DIEA (20 μL), and dimethylamine (2M THF solution, 40 μL) at 0° C. The resulting mixture was warmed to rt and stirred for 5 h. The mixture was diluted MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a yellow solid. LCMS calculated for C₂₅H₂₇ClN₉O₂ (M+H)⁺ m/z=520.2; found 520.2.

### Example 120: 9-(1-((6-Chloro-2-(1-methyl-1H-pyrazol-4-yl)pyridin-3-yl)amino)ethyl)-N,N,4,7-tetramethyl-5-oxo-4,5-dihydroimidazo[1,5-a]quinazoline-3-carboxamide

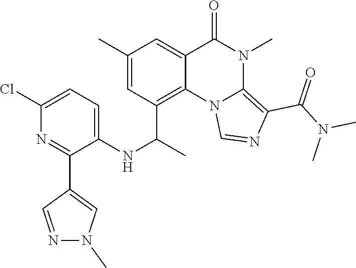 [SMILES: Cc1cc(C(C)Nc2ccc(Cl)nc2-c2cnn(C)c2)c2c(c1)c(=O)n(C)c1c(C(=O)N(C)C)ncn21]

To a solution of 9-(1-((6-chloro-2-(1-methyl-1H-pyrazol-4-yl)pyridin-3-yl)amino)ethyl)-4,7-dimethyl-5-oxo-4,5-dihydroimidazo[1,5-a]quinazoline-3-carboxylic acid (Example 116, step 1) (20 mg, 0.04 mmol) in DMF (0.5 ml) was added propanephosphonic acid anhydride (50% THF solution, 32 μL, 0.05 mmol) and DIEA (20 μL), and dimethylamine (2M THF solution, 40 μL) at 0° C. The resulting mixture was warmed to rt and stirred for 5 h. The mixture was diluted MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a yellow solid. LCMS calculated for C₂₆H₂₈ClN₈O₂ (M+H)⁺ m/z=519.2; found 519.2.

### Examples 121-122

Examples 121-122 in Table 15 were prepared similarly as described for Example 120.

TABLE 15

| Example | Chemical | | LCMS |
|---|---|---|---|
| # | name | Structure | m/z (M + H)⁺ |
| 121 | 3-(azetidine-1-carbonyl)-9-(1- ((6-chloro-2-(1-methyl-1H- pyrazol-4-yl)pyridin-3- yl)amino)ethyl)-4,7- dimethylimidazo[1,5- a]quinazolin-5(4H)-one | 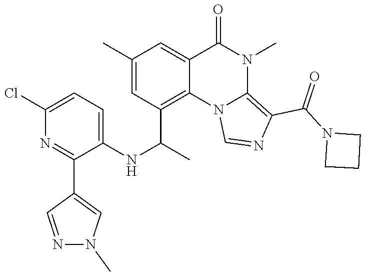 [SMILES: Cc1cc(C(C)Nc2ccc(Cl)nc2-c2cnn(C)c2)c2c(c1)c(=O)n(C)c1c(C(=O)N3CCC3)ncn21] | 531.2 |
| 122 | 9-(1-((6-chloro-2-(1-methyl- 1H-pyrazol-4-yl)pyridin-3- yl)amino)ethyl)-3-(3- hydroxyazetidine-1-carbonyl)- 4,7-dimethylimidazo[1,5- a]quinazolin-5(4H)-one | 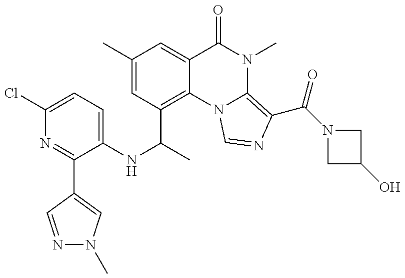 [SMILES: Cc1cc(C(C)Nc2ccc(Cl)nc2-c2cnn(C)c2)c2c(c1)c(=O)n(C)c1c(C(=O)N3CC(O)C3)ncn21] | 547.2 |

### Example 123: 9-(1-((6-chloro-2-(1-methyl-1H-1,2,4-triazol-3-yl)pyridin-3-yl)amino)ethyl)-4-(2,2-difluoroethyl)-N,N,7-trimethyl-5-oxo-4,5-dihydroimidazo[1,5-a]quinazoline-3-carboxamide

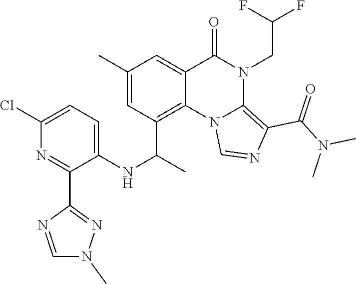 [SMILES: Cc1cc(C(C)Nc2ccc(Cl)nc2-c2ncn(C)n2)c2c(c1)c(=O)n(CC(F)F)c1c(C(=O)N(C)C)ncn21]

### Step 1: 9-(1-((6-chloro-2-(1-methyl-1H-1,2,4-triazol-3-yl)pyridin-3-yl)amino)ethyl)-4-(2,2-difluoroethyl)-7-methyl-5-oxo-4,5-dihydroimidazo[1,5-a]quinazoline-3-carboxylic acid

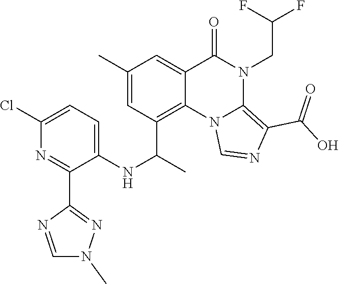 [SMILES: Cc1cc(C(C)Nc2ccc(Cl)nc2-c2ncn(C)n2)c2c(c1)c(=O)n(CC(F)F)c1c(C(=O)O)ncn21]

The title compound was prepared using similar procedures as described for Example 4 with 2,2-difluoroethan-1-amine replacing methylamine in step 3, and 6-chloro-2-(1-methyl-1H-1,2,4-triazol-3-yl)pyridin-3-amine replacing methyl 2-aminobenzoate in step 10. The crude product was used directly without further purification. LCMS calculated for C₂₄H₂₂ClF₂N₈O₃ (M+H)⁺ m/z=543.1; found 543.1.

### Step 2: 9-(1-((6-chloro-2-(1-methyl-1H-1,2,4-triazol-3-yl)pyridin-3-yl)amino)ethyl)-4-(2,2-difluoroethyl)-N,N,7-trimethyl-5-oxo-4,5-dihydroimidazo[1,5-a]quinazoline-3-carboxamide

To a solution of 9-(1-((6-chloro-2-(1-methyl-1H-1,2,4-triazol-3-yl)pyridin-3-yl)amino)ethyl)-4-(2,2-difluoroethyl)-7-methyl-5-oxo-4,5-dihydroimidazo[1,5-a]quinazoline-3-carboxylic acid (20 mg, 0.04 mmol) in DMF (0.5 ml) was added propanephosphonic acid anhydride (50% THF solution, 32 μL, 0.05 mmol) and DIEA (20 μL), and dimethylamine (2M THF solution, 40 μL) at 0° C. The resulting mixture was warmed to rt and stirred for 5 h. The mixture was diluted MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a yellow solid. LCMS calculated for C₂₆H₂₇ClF₂N₉O₂ (M+H)⁺ m/z=570.2; found 570.2.

### Example 124: 9-(1-((6-Chloro-2-(3-methyl-1H-pyrazol-4-yl)pyridin-3-yl)amino)ethyl)-N,N,4,7-tetramethyl-5-oxo-4,5-dihydroimidazo[1,5-a]quinazoline-3-carboxamide

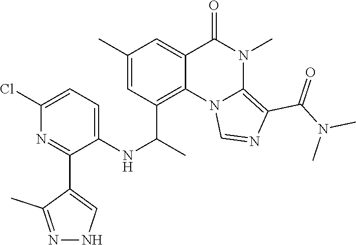

### Step 1: 9-acetyl-N,N,4,7-tetramethyl-5-oxo-4,5-dihydroimidazo[1,5-a]quinazoline-3-carboxamide

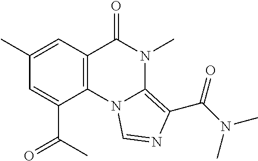

tert-Butyl 9-acetyl-4,7-dimethyl-5-oxo-4,5-dihydroimidazo[1,5-a]quinazoline-3-carboxylate (Example 4, Step 7) (1.0 g, 2.8 mmol) was dissolved in TFA (5 mL). The mixture was stirred for 3 h at rt. After completion, TFA was removed under vacuum. 50 mL of ice water and 20 mL of EtOAc were added to the reaction residue while stirring. After stirring for 20 min, the resulting solid was collected to afford a light brown solid, which was washed with water (2×). The solid was dried under vacuum to provide a beige solid. The solid product was dissolved in DMF (10 mL), then was added propanephosphonic acid anhydride (50% THF solution, 1.9 mL, 0.05 mmol) and DIEA (522 μL, 3 mmoL), and dimethylamine (2M THF solution, 1.5 mL) at 0° C. The resulting mixture was warmed to rt and stirred for 5 h. The mixture was diluted with water and extracted with DCM/IPA 3:1. The combined organic layers were washed with sat. NaCl, dried with anhydrous sodium sulfate, filtered, and concentrated under reduced pressure which was used directly in the next step. LCMS calculated for C₁₇H₁₉N₄O₃ (M+H)⁺ m/z=327.1; found 327.2.

### Step 2: 9-(1-hydroxyethyl)-N,N,4,7-tetramethyl-5-oxo-4,5-dihydroimidazo[1,5-a]quinazoline-3-carboxamide

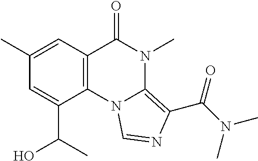

9-Acetyl-N,N,4,7-tetramethyl-5-oxo-4,5-dihydroimidazo[1,5-a]quinazoline-3-carboxamide (500 mg, 1.53 mmol) was dissolved in MeOH (10 mL) and DCM (10 mL) then NaBH₄ (60 mg, 1.6 mmol) was added at 0° C. in three portions under nitrogen. The resulting mixture was stirred at same temperature for 10 min before quenched with sat. NH₄Cl. The mixture was diluted with water and extracted with DCM. The combined organics were washed with sat. NaCl, and dried with anhydrous sodium sulfate, filtered, and concentrated under reduced pressure. The residue was purified by silica gel column chromatography, eluted with DCM and MeOH (0 to 4%) to provide the desired product as a light yellow solid (378 mg, 77%). LCMS calculated for C₁₇H₂₁N₄O₃ (M+H)⁺ m/z=329.2; found 329.2.

### Step 3: 9-(1-bromoethyl)-N,N,4,7-tetramethyl-5-oxo-4,5-dihydroimidazo[1,5-a]quinazoline-3-carboxamide

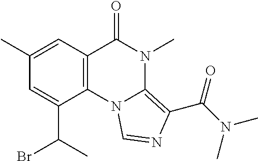

To a mixture of 9-(1-hydroxyethyl)-N,N,4,7-tetramethyl-5-oxo-4,5-dihydroimidazo[1,5-a]quinazoline-3-carboxamide (300 mg, 0.91 mmol) in DCM (200 mL) was added PBr₃ (114 μL, 1.2 mmol) at 0° C. The resulting mixture was warmed to rt and stirred for 2 h. The mixture was then quenched with sat. NaHCO₃ and extracted with DCM (2×). The combined organics were washed with sat. NaCl, and dried with anhydrous sodium sulfate, filtered, and concentrated under reduced pressure. The residue was used directly in the next step without further purification.

### Step 4: 9-(1-((6-chloro-2-(3-methyl-1H-pyrazol-4-yl)pyridin-3-yl)amino)ethyl)-N,N, 4,7-tetramethyl-5-oxo-4,5-dihydroimidazo[1,5-a]quinazoline-3-carboxamide

To the solution of 9-(1-bromoethyl)-N,N,4,7-tetramethyl-5-oxo-4,5-dihydroimidazo[1,5-a]quinazoline-3-carboxamide (10 mg, 0.025 mmol) in DMF (0.2 mL) was added tert-butyl 4-(3-amino-6-chloropyridin-2-yl)-3-methyl-1H-pyrazole-1-carboxylate (intermediate 22) (20 mg, 0.06 mmol). The mixture was heated to 80° C. for 2 h. Upon cooling to room temperature, the mixture was added TFA (0.5 mL) and stirred for 30 min, then diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₂₈ClN₈O₂ (M+H)⁺ m/z=519.2; found 519.2.

### Examples 125-130

Examples 125-130 in Table 16 were prepared similarly as described for Example 124.

TABLE 16

| Example | Chemical | | LCMS |
|---|---|---|---|
| # | name | Structure | m/z (M + H)⁺ |
| 125 | 9-(1-((6-chloro-2-(4-cyano-2- fluorophenyl)pyridin-3- yl)amino)ethyl)-N,N,4,7- tetramethyl-5-oxo-4,5- dihydroimidazo[1,5-a]quinazoline- 3-carboxamide | 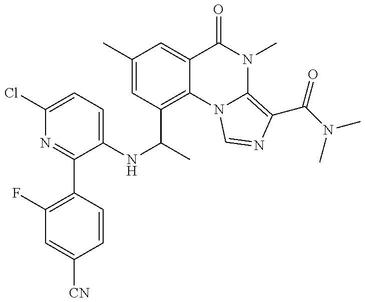 [SMILES: Cc1cc(C(C)Nc2ccc(Cl)nc2-c2ccc(C#N)cc2F)c2c(c1)c(=O)n(C)c1c(C(=O)N(C)C)ncn21] | 558.2 |
| 126 | 9-(1-((6-chloro-2-(4- cyanophenyl)pyridin-3- yl)amino)ethyl)-N,N,4,7- tetramethyl-5-oxo-4,5- dihydroimidazo[1,5-a]quinazoline- 3-carboxamide | 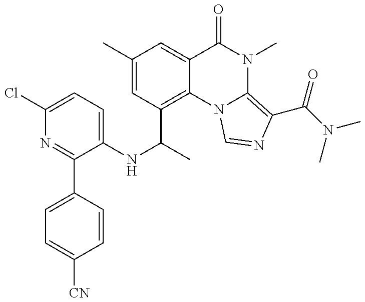 [SMILES: Cc1cc(C(C)Nc2ccc(Cl)nc2-c2ccc(C#N)cc2)c2c(c1)c(=O)n(C)c1c(C(=O)N(C)C)ncn21] | 540.2 |
| 127 | 9-(1-((6-chloro-2- morpholinopyridin-3- yl)amino)ethyl)-N,N,4,7- tetramethyl-5-oxo-4,5- dihydroimidazo[1,5-a]quinazoline- 3-carboxamide | 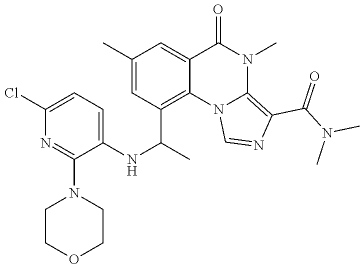 [SMILES: Cc1cc(C(C)Nc2ccc(Cl)nc2N2CCOCC2)c2c(c1)c(=O)n(C)c1c(C(=O)N(C)C)ncn21] | 524.2 |
| 128 | 9-(1-((4-fluoro-2-(1-methyl-1H- pyrazol-4-yl)phenyl)amino)ethyl)- N,N,4,7-tetramethyl-5-oxo-4,5- dihydroimidazo[1,5-a]quinazoline- 3-carboxamide | 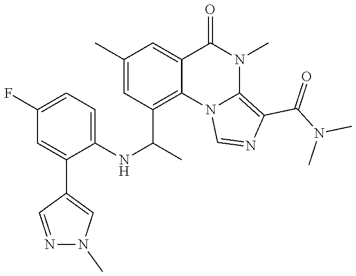 [SMILES: Cc1cc(C(C)Nc2ccc(F)cc2-c2cnn(C)c2)c2c(c1)c(=O)n(C)c1c(C(=O)N(C)C)ncn21] | 502.2 |
| 129 | 9-(1-((6-chloro-2-(1,5-dimethyl- 1H-pyrazol-4-yl)pyridin-3- yl)amino)ethyl)-N,N,4,7- tetramethyl-5-oxo-4,5- dihydroimidazo[1,5-a]quinazoline- 3-carboxamide | 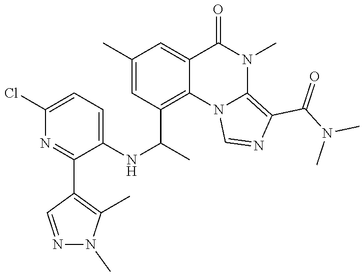 [SMILES: Cc1cc(C(C)Nc2ccc(Cl)nc2-c2cnn(C)c2C)c2c(c1)c(=O)n(C)c1c(C(=O)N(C)C)ncn21] | 533.2 |
| 130 | 9-(1-((6-chloro-2-(1,3-dimethyl- 1H-pyrazol-4-yl)pyridin-3- yl)amino)ethyl)-N,N,4,7- tetramethyl-5-oxo-4,5- dihydroimidazo[1,5-a]quinazoline- 3-carboxamide | 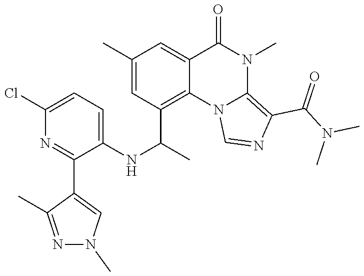 [SMILES: Cc1cc(C(C)Nc2ccc(Cl)nc2-c2cn(C)nc2C)c2c(c1)c(=O)n(C)c1c(C(=O)N(C)C)ncn21] | 533.2 |

### Example 131: 9-(1-((6-Chloro-2-(1-methyl-1H-1,2,4-triazol-3-yl)pyridin-3-yl)amino)ethyl)-4,7-dimethyl-3-(1-methylpiperidin-4-yl) imidazo[1,5-a]quinazolin-5(4H)-one

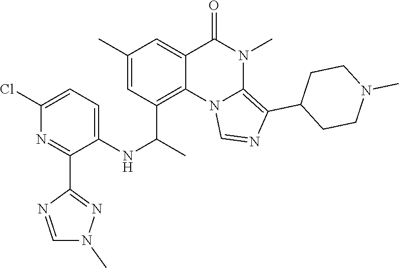 [SMILES: Cc1cc(C(C)Nc2ccc(Cl)nc2-c2ncn(C)n2)c2c(c1)c(=O)n(C)c1c(C3CCN(C)CC3)ncn21]

### Step 1: tert-butyl 4-(9-acetyl-4,7-dimethyl-5-oxo-4,5-dihydroimidazo[1,5-a]quinazolin-3-yl)-3,6-dihydropyridine-1 (2H)-carboxylate

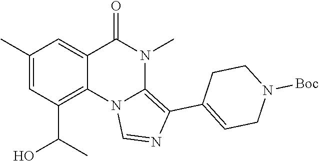 [SMILES: Cc1cc(C(C)O)c2c(c1)c(=O)n(C)c1c(C3=CCN(C(=O)OC(C)(C)C)CC3)ncn21]

A mixture of 9-acetyl-3-iodo-4,7-dimethylimidazo[1,5-a]quinazolin-5(4H)-one (Example 71, step 1) (1.0 g, 2.6 mmol), PdXPhosG2 (156 mg, 0.2 mmol), tert-butyl 4-(4,4,5,5-tetramethyl-1,3,2-dioxaborolan-2-yl)-3,6-dihydropyridine-1 (2H)-carboxylate (927 mg, 3 mmol) and K₃PO₄ (636 mg, 3 mmol) in dioxane (20 mL) and water (4 mL) was heated at 100° C. for 2 h under nitrogen atmosphere. Upon cooling to room temperature, the mixture was diluted with water and extracted with EtOAc. The combined organic layers were washed with sat. NaCl, dried with anhydrous sodium sulfate, filtered, and concentrated under reduced pressure. The residue was purified by silica gel column chromatography, eluted with DCM and ethyl acetate to provide the desired product as a brown solid (829 mg, 73%). LCMS calculated for C₂₄H₂₉N₄O₄ (M+H)⁺ m/z=437.2; found 437.2.

### Step 2: tert-butyl 4-(9-(1-hydroxyethyl)-4,7-dimethyl-5-oxo-4,5-dihydroimidazo[1,5-a]quinazolin-3-yl)-3,6-dihydropyridine-1 (2H)-carboxylate

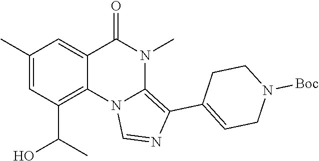 [SMILES: Cc1cc(C(C)O)c2c(c1)c(=O)n(C)c1c(C3=CCN(C(=O)OC(C)(C)C)CC3)ncn21]

To a mixture of tert-butyl 4-(9-acetyl-4,7-dimethyl-5-oxo-4,5-dihydroimidazo[1,5-a]quinazolin-3-yl)-3,6-dihydropyridine-1 (2H)-carboxylate (829 mg, 1.9 mmol) in MeOH (30 mL) and DCM (30 mL) was added NaBH₄ (114 mg, 3 mmol) at 0° C. The resulting mixture was stirred at same temperature for 10 min before being quenched with sat. NH₄Cl. The mixture was diluted with water and extracted with DCM/IPA 3:1. The combined organic layers were washed with sat. NaCl and dried with anhydrous sodium sulfate, filtered, and concentrated under reduced pressure. The residue was purified by silica gel column chromatography, eluted with DCM and MeOH to provide the desired product as a white solid (757 mg, 91%). LCMS calculated for C₂₄H₃₁N₄O₄ (M+H)⁺ m/z=439.2; found 439.2.

### Step 3: tert-butyl 4-(9-(1-hydroxyethyl)-4,7-dimethyl-5-oxo-4,5-dihydroimidazo[1,5-a]quinazolin-3-yl)piperidine-1-carboxylate

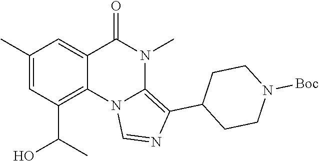 [SMILES: Cc1cc(C(C)O)c2c(c1)c(=O)n(C)c1c(C3CCN(C(=O)OC(C)(C)C)CC3)ncn21]

To a mixture of tert-butyl 4-(9-(1-hydroxyethyl)-4,7-dimethyl-5-oxo-4,5-dihydroimidazo[1,5-a]quinazolin-3-yl)-3,6-dihydropyridine-1 (2H)-carboxylate (757 mg, 1.7 mmol) in MeOH (20 mL) was added ammonium formate (630 mg, 10 mmol), Pd/C (10 wt %, 185 mg, 0.17 mmol) and acetic acid (0.5 mL) at rt under nitrogen atmosphere. The resulting mixture was stirred at 50° C. for 10 h. The mixture was filtered, and concentrated under reduced pressure. The residue was purified by silica gel column chromatography, eluted with DCM and MeOH to provide the desired product as a white solid (751 mg, 86%). LCMS calculated for C₂₄H₃₃N₄O₄ (M+H)⁺ m/z=441.2; found 441.2.

### Step 4: tert-butyl 4-(9-(1-bromoethyl)-4,7-dimethyl-5-oxo-4,5-dihydroimidazo[1,5-a]quinazolin-3-yl)piperidine-1-carboxylate

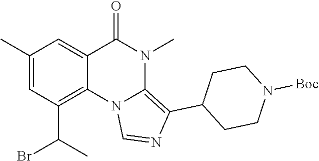 [SMILES: Cc1cc(C(C)Br)c2c(c1)c(=O)n(C)c1c(C3CCN(C(=O)OC(C)(C)C)CC3)ncn21]

To a mixture of tert-butyl 4-(9-(1-hydroxyethyl)-4,7-dimethyl-5-oxo-4,5-dihydroimidazo[1,5-a]quinazolin-3-yl)piperidine-1-carboxylate (300 mg, 0.68 mmol) in THF (20 mL) was added pyridine (0.22 mL, 2.72 mmol), PBr₃ (367 mg, 1.38 mmol). The resulting mixture was stirred at rt for 2 h. The mixture was then quenched with sat. NaHCO₃ and extracted with EtOAc. The combined organic layers were washed with sat. NaCl, dried with anhydrous sodium sulfate, filtered, and concentrated under reduced pressure. The residue was used directly in the next step without further purification.

### Step 5: 9-(1-((6-chloro-2-(1-methyl-1H-1,2,4-triazol-3-yl)pyridin-3-yl)amino)ethyl)-4,7-dimethyl-3-(piperidin-4-yl) imidazo[1,5-a]quinazolin-5(4H)-one

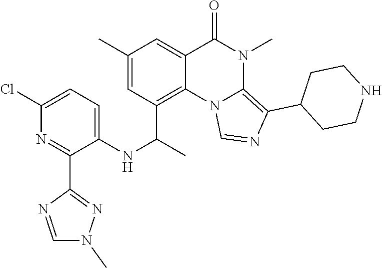 [SMILES: Cc1cc(C(C)Nc2ccc(Cl)nc2-c2ncn(C)n2)c2c(c1)c(=O)n(C)c1c(C3CCNCC3)ncn21]

tert-Butyl 4-(9-(1-bromoethyl)-4,7-dimethyl-5-oxo-4,5-dihydroimidazo[1,5-a]quinazolin-3-yl)piperidine-1-carboxylate (60 mg, 0.12 mmol) in 2 dram vial was added DMF (0.3 mL), and 6-chloro-2-(1-methyl-1H-1,2,4-triazol-3-yl)pyridin-3-amine (60 mg, 0.30 mmol). The mixture was heated to 80° C. for 1 h. Upon cooling to room temperature, to the mixture was added TFA (0.5 mL) and stirred for 30 min before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₁ClN₉O (M+H)⁺ m/z=532.2; found 532.2.

### Step 6: 9-(1-((6-chloro-2-(1-methyl-1H-1,2,4-triazol-3-yl)pyridin-3-yl)amino)ethyl)-4,7-dimethyl-3-(1-methylpiperidin-4-yl) imidazo[1,5-a]quinazolin-5(4H)-one

To a mixture of 9-(1-((6-chloro-2-(1-methyl-1H-1,2,4-triazol-3-yl)pyridin-3-yl)amino)ethyl)-4,7-dimethyl-3-(piperidin-4-yl) imidazo[1,5-a]quinazolin-5(4H)-one (10 mg, 0.02 mmol) was added DCM (0.5 mL), formaldehyde (water solution, 10 μL) and sodium triacetoxyborohydride (10 mg, 0.04 mmol). The mixture was stirred for 30 min, then diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₃₃ClN₉O (M+H)⁺ m/z=546.2; found 546.2.

### Example 132: 3-(1-Acetylpiperidin-4-yl)-9-(1-((6-chloro-2-(1-methyl-1H-1,2,4-triazol-3-yl)pyridin-3-yl)amino)ethyl)-4,7-dimethylimidazo[1,5-a]quinazolin-5(4H)-one

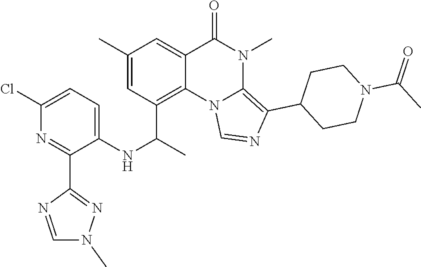 [SMILES: CC(=O)N1CCC(c2ncn3c4c(C(C)Nc5ccc(Cl)nc5-c5ncn(C)n5)cc(C)cc4c(=O)n(C)c23)CC1]

To a mixture of 9-(1-((6-chloro-2-(1-methyl-1H-1,2,4-triazol-3-yl)pyridin-3-yl)amino)ethyl)-4,7-dimethyl-3-(piperidin-4-yl) imidazo[1,5-a]quinazolin-5(4H)-one (Example 131, step 5) (10 mg, 0.019 mmol) in DCM (0.5 mL) was added acetyl chloride (3.2 mg, 0.04 mmol) DCM (0.2 mL) solution and N,N-diisopropylethylamine (5.0 μL) at 0° C. The reaction mixture was stirred at same temperature for 30 minutes before the solvent was removed under vacuum. The resulting mixture was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₃ClN₉O₂ (M+H)⁺ m/z=574.2; found 574.2.

### Example 133

Example 133 in Table 17 were prepared similarly as described for Example 132.

TABLE 17

| Example | Chemical | | LCMS |
|---|---|---|---|
| # | name | Structure | m/z (M + H)⁺ |
| 133 | ethyl 4-(9-(1-((6- chloro-2-(1-methyl- 1H-1,2,4-triazol-3- yl)pyridin-3- yl)amino)ethyl)-4,7- dimethyl-5-oxo-4,5- dihydroimidazo[1,5- a]quinazolin-3- yl)piperidine-1- carboxylate | 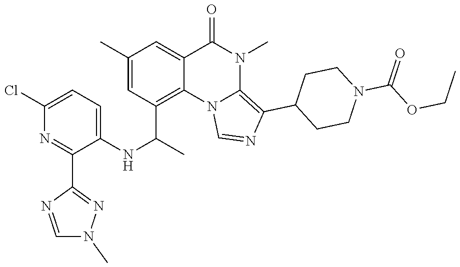 [SMILES: CCOC(=O)N1CCC(c2ncn3c4c(C(C)Nc5ccc(Cl)nc5-c5ncn(C)n5)cc(C)cc4c(=O)n(C)c23)CC1] | 604.2 |

### Example 134: 9-(1-((6-Chloro-2-(1-methyl-1H-pyrazol-4-yl)pyridin-3-yl)amino)ethyl)-4,7-dimethyl-3-(1-methylpiperidin-4-yl) imidazo[1,5-a]quinazolin-5(4H)-one

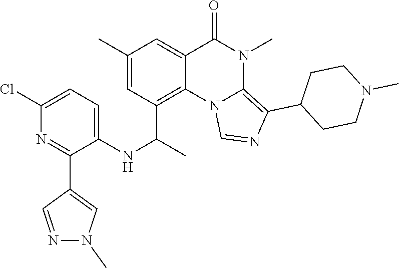 [SMILES: Cc1cc(C(C)Nc2ccc(Cl)nc2-c2cnn(C)c2)c2c(c1)c(=O)n(C)c1c(C3CCN(C)CC3)ncn21]

The titled compound was prepared using similar procedures as described for Example 131 with 6-chloro-2-(1-methyl-1H-pyrazol-4-yl)pyridin-3-amine replacing 6-chloro-2-(1-methyl-1H-1,2,4-triazol-3-yl)pyridin-3-amine in step 5. The crude product was used directly without further purification. LCMS calculated for C₂₉H₃₄ClN₈O (M+H)⁺ m/z=545.3; found 545.2.

### Example 135: 9-(1-((6-Chloro-2-(1-methyl-1H-pyrazol-4-yl)pyridin-3-yl)amino)ethyl)-4,7-dimethyl-3-(1-methylpyrrolidin-3-yl)-3,4-dihydro-5H-pyrazolo[3,4-c]isoquinolin-5-one

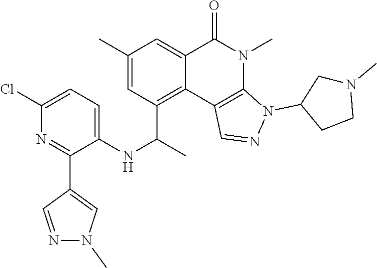 [SMILES: Cc1cc(C(C)Nc2ccc(Cl)nc2-c2cnn(C)c2)c2c(c1)c(=O)n(C)c1c2cnn1C1CCN(C)C1]

### Step 1: benzyl 3-(9-(1-bromoethyl)-4,7-dimethyl-5-oxo-4,5-dihydro-3H-pyrazolo[3,4-c]isoquinolin-3-yl) pyrrolidine-1-carboxylate

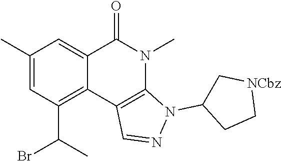 [SMILES: Cc1cc(C(C)Br)c2c(c1)c(=O)n(C)c1c2cnn1C1CCN(C(=O)OCc2ccccc2)C1]

The titled compound was prepared using similar procedures as described for Example 81 with phenylmethyl 3-[(methylsulfonyl)oxy]-1-pyrrolidinecarboxylate replacing tert-butyl 3-((methylsulfonyl)oxy) azetidine-1-carboxylate in Step 1.

### Step 2: 9-(1-((6-chloro-2-(1-methyl-1H-pyrazol-4-yl)pyridin-3-yl)amino)ethyl)-4,7-dimethyl-3-(pyrrolidin-3-yl)-3,4-dihydro-5H-pyrazolo[3,4-c]isoquinolin-5-one

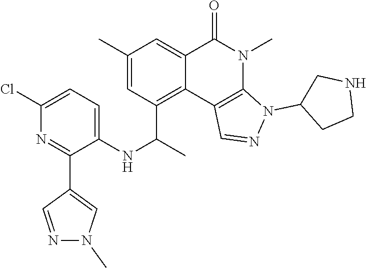 [SMILES: Cc1cc(C(C)Nc2ccc(Cl)nc2-c2cnn(C)c2)c2c(c1)c(=O)n(C)c1c2cnn1C1CCNC1]

Benzyl 3-(9-(1-bromoethyl)-4,7-dimethyl-5-oxo-4,5-dihydro-3H-pyrazolo[3,4-c]isoquinolin-3-yl) pyrrolidine-1-carboxylate (58 mg, 0.11 mmol) in 2 dram vial was added DMF (0.2 mL), and 6-chloro-2-(1-methyl-1H-pyrazol-4-yl)pyridin-3-amine (20 mg, 0.096 mmol). The mixture was heated at 80° C. for 1 h. Upon cooling to room temperature, the mixture was added HBr (48 wt. % in acetic acid, 1 mL) and stirred for 30 min at 0° C. The resulting mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₀ClN₈O_{(M+H)}⁺ m/z=517.2; found 517.2.

### Step 3: 9-(1-((6-chloro-2-(1-methyl-1H-pyrazol-4-yl)pyridin-3-yl)amino)ethyl)-4,7-dimethyl-3-(1-methylpyrrolidin-3-yl)-3,4-dihydro-5H-pyrazolo[3,4-c]isoquinolin-5-one

To a mixture of 9-(1-((6-chloro-2-(1-methyl-1H-pyrazol-4-yl)pyridin-3-yl)amino)ethyl)-4,7-dimethyl-3-(pyrrolidin-3-yl)-3,4-dihydro-5H-pyrazolo[3,4-c]isoquinolin-5-one (5 mg, 0.01 mmol) was added DCM (0.5 mL), formaldehyde (water solution, 10 μL) and sodium triacetoxyborohydride (4.2 mg, 0.02 mmol). The mixture was stirred for 30 min, then diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₃₂ClN₈O (M+H)⁺ m/z=531.2; found 531.2.

### Examples 136-141

Examples 136-141 in Table 18 were prepared similarly as described for Example 135.

TABLE 18

| Example | Chemical | | LCMS |
|---|---|---|---|
| # | name | Structure | m/z (M + H)⁺ |
| 136 | 9-(1-((6-chloro-2-(1- methyl-1H-1,2,4-triazol- 3-yl)pyridin-3- yl)amino)ethyl)-4,7- dimethyl-3-(1- methylpyrrolidin-3-yl)- 3,4-dihydro-5H- pyrazolo[3,4- c]isoquinolin-5-one | 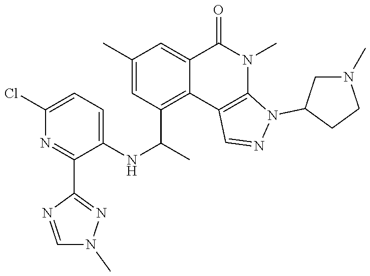 [SMILES: Cc1cc(C(C)Nc2ccc(Cl)nc2-c2ncn(C)n2)c2c(c1)c(=O)n(C)c1c2cnn1C1CCN(C)C1] | 532.2 |
| 137 | 9-(1-((6-chloro-2-(1- methyl-1H-1,2,4-triazol- 3-yl)pyridin-3- yl)amino)ethyl)-3-(1- ethylpyrrolidin-3-yl)-4,7- dimethyl-3,4-dihydro-5H- pyrazolo[3,4- c]isoquinolin-5-one | 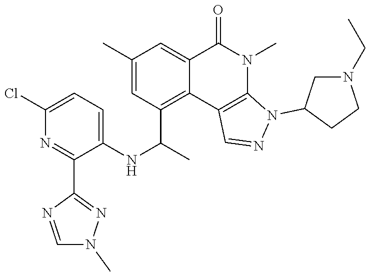 [SMILES: CCN1CCC(n2ncc3c4c(C(C)Nc5ccc(Cl)nc5-c5ncn(C)n5)cc(C)cc4c(=O)n(C)c32)C1] | 546.2 |
| 138 | 9-(1-((6-chloro-2-(1- methyl-1H-1,2,4-triazol- 3-yl)pyridin-3- yl)amino)ethyl)-3-(1-(2- hydroxyethyl)pyrrolidin- 3-yl)-4,7-dimethyl-3,4- dihydro-5H-pyrazolo[3,4- c]isoquinolin-5-one | 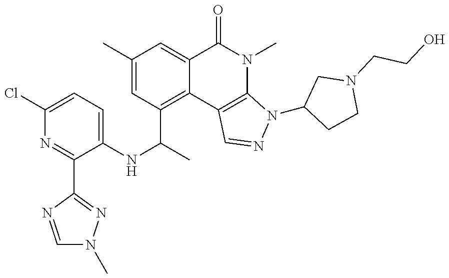 [SMILES: Cc1cc(C(C)Nc2ccc(Cl)nc2-c2ncn(C)n2)c2c(c1)c(=O)n(C)c1c2cnn1C1CCN(CCO)C1] | 562.2 |
| 139 | 9-(1-((6-chloro-2-(2- methyl-2H-tetrazol-5- yl)pyridin-3- yl)amino)ethyl)-7-methyl- 4-(methyl-d3)-3-(1- methylpyrrolidin-3-yl)- 3,4-dihydro-5H- pyrazolo[3,4- c]isoquinolin-5-one | 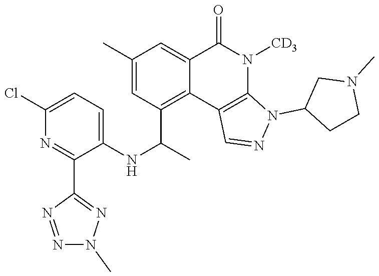 [SMILES: [2H]C([2H])([2H])n1c(=O)c2cc(C)cc(C(C)Nc3ccc(Cl)nc3-c3nnn(C)n3)c2c2cnn(C3CCN(C)C3)c21] | 536.2 |
| 140 | 9-(1-((6-chloro-2-(1- methyl-1H-pyrazol-4- yl)pyridin-3- yl)amino)ethyl)-3-(1-(2- hydroxyethyl)pyrrolidin- 3-yl)-7-methyl-4-(methyl- d3)-3,4-dihydro-5H- pyrazolo[3,4- c]isoquinolin-5-one | 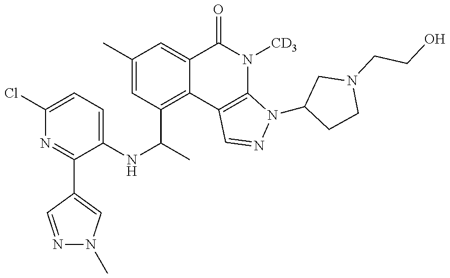 [SMILES: [2H]C([2H])([2H])n1c(=O)c2cc(C)cc(C(C)Nc3ccc(Cl)nc3-c3cnn(C)c3)c2c2cnn(C3CCN(CCO)C3)c21] | 564.3 |
| 141 | 9-(1-((6-chloro-3′-fluoro- [2,4′-bipyridin]-3- yl)amino)ethyl)-3-(1- isopropylpyrrolidin-3-yl)- 4,7-dimethyl-3,4-dihydro- 5H-pyrazolo[3,4- c]isoquinolin-5-one | 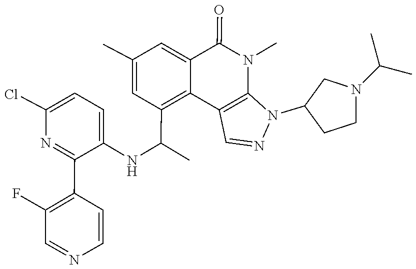 [SMILES: Cc1cc(C(C)Nc2ccc(Cl)nc2-c2ccncc2F)c2c(c1)c(=O)n(C)c1c2cnn1C1CCN(C(C)C)C1] | 574.2 |

### Example 142: 9-(1-((6-Chloro-2-(1-methyl-1H-pyrazol-4-yl)pyridin-3-yl)amino)ethyl)-3-(1-(2,2-difluoroethyl) pyrrolidin-3-yl)-4,7-dimethyl-3,4-dihydro-5H-pyrazolo[3,4-c]isoquinolin-5-one

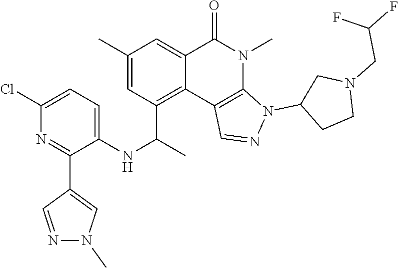 [SMILES: Cc1cc(C(C)Nc2ccc(Cl)nc2-c2cnn(C)c2)c2c(c1)c(=O)n(C)c1c2cnn1C1CCN(CC(F)F)C1]

To a solution of 9-(1-((6-chloro-2-(1-methyl-1H-pyrazol-4-yl)pyridin-3-yl)amino)ethyl)-4,7-dimethyl-3-(pyrrolidin-3-yl)-3,4-dihydro-5H-pyrazolo[3,4-c]isoquinolin-5-one (Example 135, step 2) (5 mg, 0.01 mmol) in DMF (0.2 mL) was added 2,2-difluoroethyl trifluoromethanesulfonate (10.4 mg, 0.05 mmol) and N,N-diisopropylethylamine (6.3 mg, 0.05 mmol). The reaction was stirred at room temperature for 16 hours. The resulting mixture was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₂ClF₂N₈O (M+H)⁺ m/z=581.2; found 581.2.

### Examples 143-144

Examples 143-144 in Table 19 were prepared similarly as described for Example 142.

TABLE 19

| Ex- | | | LCMS |
|---|---|---|---|
| ample | | | m/z |
| # | Chemical name | Structure | (M + H)⁺ |
| 143 | 9-(1-((6-chloro-2-(1- methyl-1H-pyrazol-4- yl)pyridin-3- yl)amino)ethyl)-3-(1-(2,2- difluoroethyl)pyrrolidin-3- yl)-7-methyl-4-(methyl- d3)-3,4-dihydro-5H- pyrazolo[3,4-c]isoquinolin- 5-one | 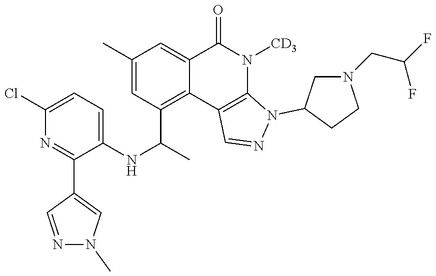 [SMILES: [2H]C([2H])([2H])n1c(=O)c2cc(C)cc(C(C)Nc3ccc(Cl)nc3-c3cnn(C)c3)c2c2cnn(C3CCN(CC(F)F)C3)c21] | 584.3 |
| 144 | 2-(3-(9-(1-((6-chloro-2-(1- methyl-1H-pyrazol-4- yl)pyridin-3- yl)amino)ethyl)-7-methyl- 4-(methyl-d₃)-5-oxo-4,5- dihydro-3H-pyrazolo[3,4- c]isoquinolin-3- yl)pyrrolidin-1- yl)acetonitrile | 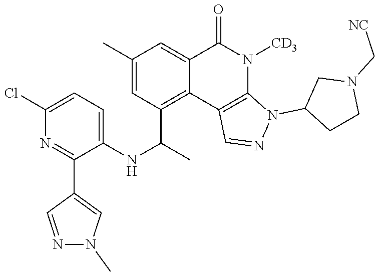 [SMILES: [2H]C([2H])([2H])n1c(=O)c2cc(C)cc(C(C)Nc3ccc(Cl)nc3-c3cnn(C)c3)c2c2cnn(C3CCN(CC#N)C3)c21] | 559.2 |

### Example 145:9-(1-((6-Chloro-2-(1-methyl-1H-1,2,4-triazol-3-yl)pyridin-3-yl)amino)ethyl)-4-ethyl-3-(1-(2-hydroxyethyl) pyrrolidin-3-yl)-7-methyl-3,4-dihydro-5H-pyrazolo[3,4-c]isoquinolin-5-one

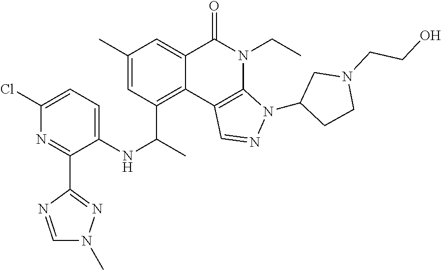 [SMILES: CCn1c(=O)c2cc(C)cc(C(C)Nc3ccc(Cl)nc3-c3ncn(C)n3)c2c2cnn(C3CCN(CCO)C3)c21]

### Step 1: 9-(1-((6-chloro-2-(1-methyl-1H-1,2,4-triazol-3-yl)pyridin-3-yl)amino)ethyl)-4-ethyl-7-methyl-3-(pyrrolidin-3-yl)-3,4-dihydro-5H-pyrazolo[3,4-c]isoquinolin-5-one

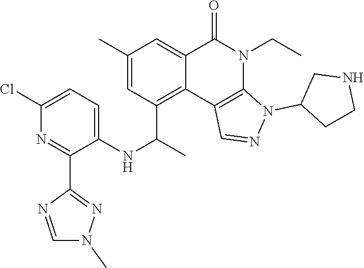 [SMILES: CCn1c(=O)c2cc(C)cc(C(C)Nc3ccc(Cl)nc3-c3ncn(C)n3)c2c2cnn(C3CCNC3)c21]

The titled compound was prepared using similar procedures as described for Example 81 with phenylmethyl 3-[(methylsulfonyl)oxy]-1-pyrrolidinecarboxylate replacing tert-butyl 3-((methylsulfonyl)oxy) azetidine-1-carboxylate in Step 1, iodoethane replacing iodomethane in step 5, and 6-chloro-2-(1-methyl-1H-1,2,4-triazol-3-yl)pyridin-3-amine replacing 6-chloro-2-(1-methyl-1H-pyrazol-4-yl)pyridin-3-amine in Step 10. The crude product was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₁ClN₉O (M+H)⁺ m/z=532.2; found 532.2.

### Step 2: 9-(1-((6-chloro-2-(1-methyl-1H-1,2,4-triazol-3-yl)pyridin-3-yl)amino)ethyl)-4-ethyl-3-(1-(2-hydroxyethyl) pyrrolidin-3-yl)-7-methyl-3,4-dihydro-5H-pyrazolo[3,4-c]isoquinolin-5-one

To a mixture of 9-(1-((6-chloro-2-(1-methyl-1H-1,2,4-triazol-3-yl)pyridin-3-yl)amino)ethyl)-4-ethyl-7-methyl-3-(pyrrolidin-3-yl)-3,4-dihydro-5H-pyrazolo[3,4-c]isoquinolin-5-one (7 mg, 0.01 mmol) was added DCM (0.5 mL), 2-((tert-butyldimethylsilyl)oxy) acetaldehyde (10 mg, 0.05 mmol) and sodium triacetoxyborohydride (5.6 mg, 0.03 mmol). The mixture was stirred for 30 min, solvent was removed under vacuum. 4 M HCl in dioxane (0.3 mL) and MeOH (0.2 mL) were added and the mixture was stirred for 10 min before being diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₅ClN₉O₂ (M+H)⁺ m/z=576.2; found 576.2.

### Examples 146-147

Examples 146-147 in Table 20 were prepared similarly as described for Example 145.

TABLE 20

| Ex- | | | LCMS |
|---|---|---|---|
| ample | | | m/z |
| # | Chemical name | Structure | (M + H)⁺ |
| 146 | 9-(1-((6-chloro-2-(1- methyl-1H-1,2,4-triazol- 3-yl)pyridin-3- yl)amino)ethyl)-4-ethyl- 3-(1-(2-hydroxy-2- methylpropyl)pyrrolidin- 3-yl)-7-methyl-3,4- dihydro-5H- pyrazolo[3,4- c]isoquinolin-5-one | 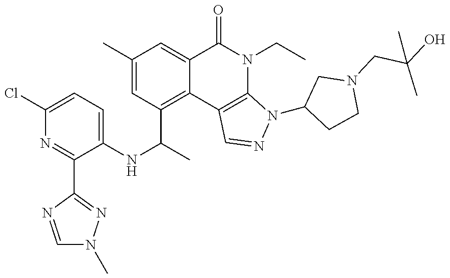 [SMILES: CCn1c(=O)c2cc(C)cc(C(C)Nc3ccc(Cl)nc3-c3ncn(C)n3)c2c2cnn(C3CCN(CC(C)(C)O)C3)c21] | 604.3 |
| 147 | 9-(1-((6-chloro-2-(1- methyl-1H-1,2,4-triazol- 3-yl)pyridin-3- yl)amino)ethyl)-4-ethyl- 3-(1-isopropylpyrrolidin- 3-yl)-7-methyl-3,4- dihydro-5H- pyrazolo[3,4- c]isoquinolin-5-one | 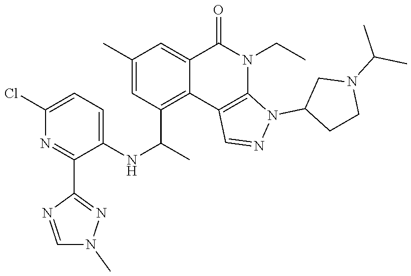 [SMILES: CCn1c(=O)c2cc(C)cc(C(C)Nc3ccc(Cl)nc3-c3ncn(C)n3)c2c2cnn(C3CCN(C(C)C)C3)c21] | 574.3 |

### Example 148: 9-(1-((6-Chloro-2-(1-methyl-1H-1,2,4-triazol-3-yl)pyridin-3-yl)amino)ethyl)-4-ethyl-7-methyl-3-(1-(methylsulfonyl) pyrrolidin-3-yl)-3,4-dihydro-5H-pyrazolo[3,4-c]isoquinolin-5-one

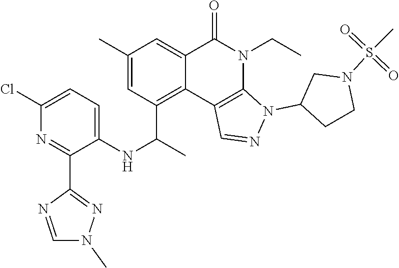 [SMILES: CCn1c(=O)c2cc(C)cc(C(C)Nc3ccc(Cl)nc3-c3ncn(C)n3)c2c2cnn(C3CCN(S(C)(=O)=O)C3)c21]

To a solution of 9-(1-((6-chloro-2-(1-methyl-1H-1,2,4-triazol-3-yl)pyridin-3-yl)amino)ethyl)-4-ethyl-7-methyl-3-(pyrrolidin-3-yl)-3,4-dihydro-5H-pyrazolo[3,4-c]isoquinolin-5-one (Example 145, step 1) (5.0 mg, 0.01 mmol) in DCM (0.5 mL) was added methanesulfonyl chloride (4.0 mg, 0.04 mmol) and N,N-diisopropylethylamine (2.4 mg, 0.02 mmol). The reaction mixture was stirred at room temperature for 30 minutes before the solvent was removed under vacuum. The resulting mixture was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₃₃ClN₉O₃S (M+H)⁺ m/z=610.2; found 610.2.

### Examples 149-151

Examples 149-151 in Table 21 were prepared similarly as described for Example 148.

TABLE 21

| Ex | | | LCMS |
|---|---|---|---|
| ample | | | m/z |
| # | Chemical name | Structure | (M + H)⁺ |
| 149 | 3-(1-acetylpyrrolidin-3-yl)-9- (1-((6-chloro-2-(1-methyl-1H- 1,2,4-triazol-3-yl)pyridin-3- yl)amino)ethyl)-4-ethyl-7- methyl-3,4-dihydro-5H- pyrazolo[3,4-c]isoquinolin-5- one | 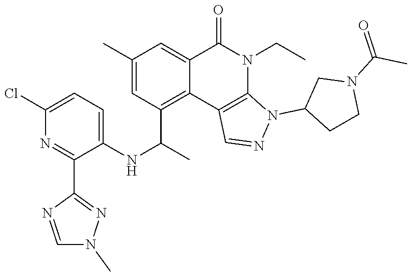 [SMILES: CCn1c(=O)c2cc(C)cc(C(C)Nc3ccc(Cl)nc3-c3ncn(C)n3)c2c2cnn(C3CCN(C(C)=O)C3)c21] | 574.2 |
| 150 | methyl 3-(9-(1-((6-chloro-2- (1-methyl-1H-1,2,4-triazol-3- yl)pyridin-3-yl)amino)ethyl)- 4-ethyl-7-methyl-5-oxo-4,5- dihydro-3H-pyrazolo[3,4- c]isoquinolin-3-yl)pyrrolidine- 1-carboxylate | 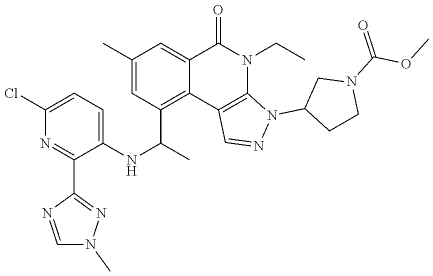 [SMILES: CCn1c(=O)c2cc(C)cc(C(C)Nc3ccc(Cl)nc3-c3ncn(C)n3)c2c2cnn(C3CCN(C(=O)OC)C3)c21] | 590.2 |
| 151 | 3-(9-(1-((6-chloro-3′-fluoro- [2,4′-bipyridin]-3- yl)amino)ethyl)-4,7-dimethyl- 5-oxo-4,5-dihydro-3H- pyrazolo[3,4-c]isoquinolin-3- yl)-N,N-dimethylpyrrolidine- 1-carboxamide | 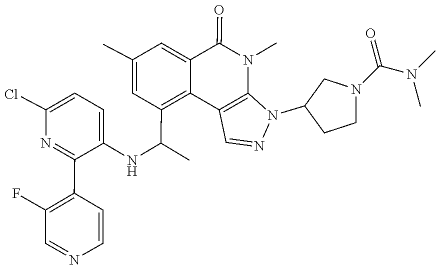 [SMILES: Cc1cc(C(C)Nc2ccc(Cl)nc2-c2ccncc2F)c2c(c1)c(=O)n(C)c1c2cnn1C1CCN(C(=O)N(C)C)C1] | 603.2 |

### Example 152: 3-(3-(9-(1-((6-Chloro-2-(1-methyl-1H-pyrazol-4-yl)pyridin-3-yl)amino)ethyl)-7-methyl-4-(methyl-d3)-5-oxo-4,5-dihydro-3H-pyrazolo[3,4-c]isoquinolin-3-yl) pyrrolidin-1-yl)propanenitrile

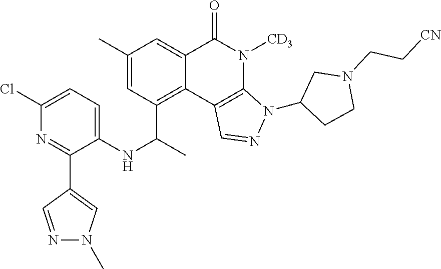 [SMILES: [2H]C([2H])([2H])n1c(=O)c2cc(C)cc(C(C)Nc3ccc(Cl)nc3-c3cnn(C)c3)c2c2cnn(C3CCN(CCC#N)C3)c21]

### Step 1: 9-(1-((6-chloro-2-(1-methyl-1H-pyrazol-4-yl)pyridin-3-yl)amino)ethyl)-7-methyl-4-(methyl-d3)-3-(pyrrolidin-3-yl)-3,4-dihydro-5H-pyrazolo[3,4-c]isoquinolin-5-one

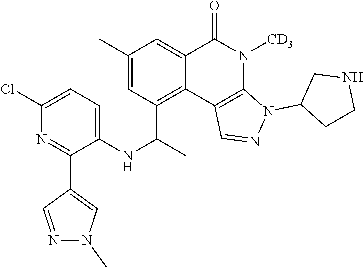 [SMILES: [2H]C([2H])([2H])n1c(=O)c2cc(C)cc(C(C)Nc3ccc(Cl)nc3-c3cnn(C)c3)c2c2cnn(C3CCNC3)c21]

The titled compound was prepared using similar procedures as described for Example 81 with phenylmethyl 3-[(methylsulfonyl)oxy]-1-pyrrolidinecarboxylate replacing tert-butyl 3-((methylsulfonyl)oxy) azetidine-1-carboxylate in Step 1, iodomethane-d3 replacing iodomethane in step 5. The crude product was deprotected with neat 30% HBr AcOH solution and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₂₇D₃ClN₈O (M+H)⁺ m/z=520.2; found 520.2.

### Step 2: 3-(3-(9-(1-((6-chloro-2-(1-methyl-1H-pyrazol-4-yl)pyridin-3-yl)amino)ethyl)-7-methyl-4-(methyl-d3)-5-oxo-4,5-dihydro-3H-pyrazolo[3,4-c]isoquinolin-3-yl) pyrrolidin-1-yl)propanenitrile

To a solution of 9-(1-((6-chloro-2-(1-methyl-1H-pyrazol-4-yl)pyridin-3-yl)amino)ethyl)-7-methyl-4-(methyl-d3)-3-(pyrrolidin-3-yl)-3,4-dihydro-5H-pyrazolo[3,4-c]isoquinolin-5-one (10 mg, 0.02 mmol) in methanol (0.5 mL) was added acrylonitrile (5.1 mg, 0.1 mmol) and triethylamine (9.7 mg, 0.1 mmol). The reaction mixture was stirred at 50° C. for 6 hours. Upon cooling to room temperature, the resulting mixture was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₀H₃₀D₃ClN₉O (M+H)⁺ m/z=573.2; found 573.2.

### Example 153

Example 153 in Table 22 were prepared similarly as described for Example 152.

TABLE 22

| Ex- | | | LCMS |
|---|---|---|---|
| ample | | | m/z |
| # | Chemical name | Structure | (M + H)⁺ |
| 153 | 3-(3-(9-(1-((6-chloro-2-(2- methyl-2H-tetrazol-5- yl)pyridin-3-yl)amino)ethyl)-7- methyl-4-(methyl-d3)-5-oxo- 4,5-dihydro-3H-pyrazolo[3,4- c]isoquinolin-3-yl)pyrrolidin- 1-yl)propanenitrile | 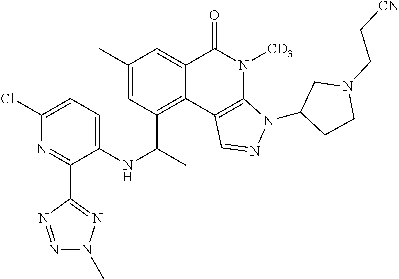 [SMILES: [2H]C([2H])([2H])n1c(=O)c2cc(C)cc(C(C)Nc3ccc(Cl)nc3-c3nnn(C)n3)c2c2cnn(C3CCN(CCC#N)C3)c21] | 575.2 |

### Example 154: 9-(1-((6-Chloro-2-(1-methyl-1H-pyrazol-4-yl)pyridin-3-yl)amino)ethyl)-3-(1-(2-hydroxyacetyl) pyrrolidin-3-yl)-7-methyl-4-(methyl-d3)-3,4-dihydro-5H-pyrazolo[3,4-c]isoquinolin-5-one

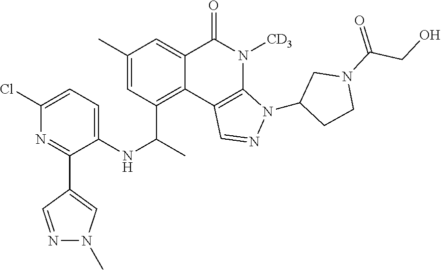 [SMILES: [2H]C([2H])([2H])n1c(=O)c2cc(C)cc(C(C)Nc3ccc(Cl)nc3-c3cnn(C)c3)c2c2cnn(C3CCN(C(=O)CO)C3)c21]

To a solution of 9-(1-((6-chloro-2-(1-methyl-1H-pyrazol-4-yl)pyridin-3-yl)amino)ethyl)-7-methyl-4-(methyl-d3)-3-(pyrrolidin-3-yl)-3,4-dihydro-5H-pyrazolo[3,4-c]isoquinolin-5-one (Example 152, step 1) (10 mg, 0.02 mmol) in DMF (0.2 mL) was added 2-hydroxyacetic acid (2.9 mg, 0.04 mmol), HATU (14.6 mg, 0.04 mmol) and N,N-diisopropylethylamine (7.5 mg, 0.06). The reaction mixture was stirred at room temperature for 30 minutes. The resulting mixture was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₂₉D₃ClN₈O₃ (M+H)⁺ m/z=578.2; found 578.2.

### Examples 155-157

Examples 155-157 in Table 23 were prepared similarly as described for Example 154.

TABLE 23

| Ex- | | | LCMS |
|---|---|---|---|
| ample | | | m/z |
| # | Chemical name | Structure | (M + H)⁺ |
| 155 | 9-(1-((6-chloro-2-(1- methyl-1H-pyrazol-4- yl)pyridin-3- yl)amino)ethyl)-3-(1- (dimethylglycyl)pyrrolidin- 3-yl)-7-methyl-4-(methyl- d₃)-3,4-dihydro-5H- pyrazolo[3,4-c]isoquinolin- 5-one | 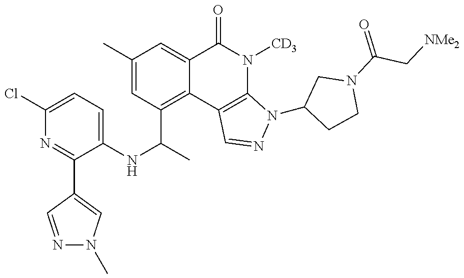 [SMILES: [2H]C([2H])([2H])n1c(=O)c2cc(C)cc(C(C)Nc3ccc(Cl)nc3-c3cnn(C)c3)c2c2cnn(C3CCN(C(=O)CN(C)C)C3)c21] | 605.3 |
| 156 | 9-(1-((6-chloro-2-(1- methyl-1H-pyrazol-4- yl)pyridin-3- yl)amino)ethyl)-7-methyl- 4-(methyl-d3)-3-(1-(1- methyl-1H-pyrazole-5- carbonyl)pyrrolidin-3-yl)- 3,4-dihydro-5H- pyrazolo[3,4-c]isoquinolin- 5-one | 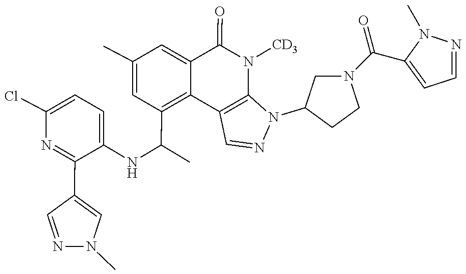 [SMILES: [2H]C([2H])([2H])n1c(=O)c2cc(C)cc(C(C)Nc3ccc(Cl)nc3-c3cnn(C)c3)c2c2cnn(C3CCN(C(=O)c4ccnn4C)C3)c21] | 628.3 |
| 157 | 1-(3-(9-(1-((6-chloro-2-(1- methyl-1H-pyrazol-4- yl)pyridin-3- yl)amino)ethyl)-7-methyl- 4-(methyl-d3)-5-oxo-4,5- dihydro-3H-pyrazolo[3,4- c]isoquinolin-3- yl)pyrrolidine-1- carbonyl)cyclopropane-1- carbonitrile | 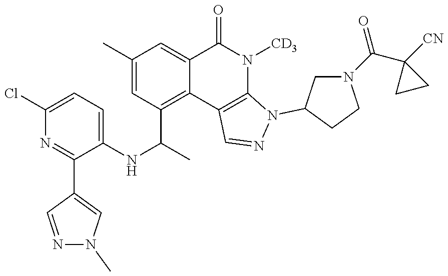 [SMILES: [2H]C([2H])([2H])n1c(=O)c2cc(C)cc(C(C)Nc3ccc(Cl)nc3-c3cnn(C)c3)c2c2cnn(C3CCN(C(=O)C4(C#N)CC4)C3)c21] | 613.3 |

### Example 158: 9-(1-((6-Chloro-2-(1-methyl-1H-pyrazol-4-yl)pyridin-3-yl)amino)ethyl)-4,7-dimethyl-3-(1-methylpiperidin-4-yl)-3,4-dihydro-5H-pyrazolo[3,4-c]isoquinolin-5-one

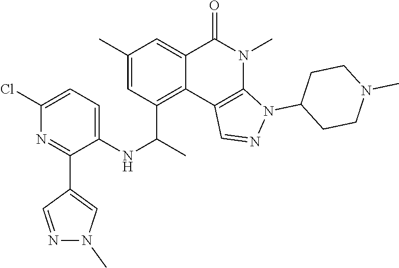 [SMILES: Cc1cc(C(C)Nc2ccc(Cl)nc2-c2cnn(C)c2)c2c(c1)c(=O)n(C)c1c2cnn1C1CCN(C)CC1]

### Step 1: benzyl 4-[9-(1-bromoethyl)-4,7-dimethyl-5-oxo-pyrazolo[3,4-c]isoquinolin-3-yl]piperidine-1-carboxylate

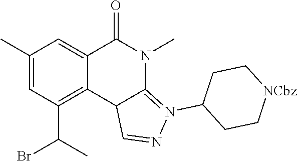

The titled compound was prepared using similar procedures as described for Example 81 with benzyl 4-hydrazinopiperidine-1-carboxylate replacing tert-butyl 3-hydrazineylazetidine-1-carboxylate in Step 2. The resulting residue was used directly in next step without further purification.

### Step 2: benzyl 4-[9-[1-[[6-chloro-2-(1-methylpyrazol-4-yl)-3-pyridyl]amino]ethyl]-4,7-dimethyl-5-oxo-pyrazolo[3,4-c]isoquinolin-3-yl]piperidine-1-carboxylate

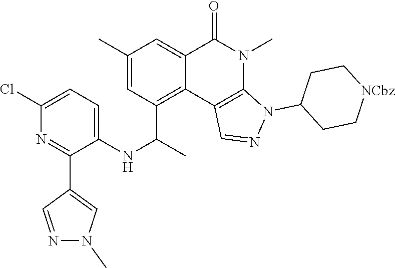 [SMILES: Cc1cc(C(C)Nc2ccc(Cl)nc2-c2cnn(C)c2)c2c(c1)c(=O)n(C)c1c2cnn1C1CCN(C(=O)OCc2ccccc2)CC1]

To a solution of benzyl 4-[9-(1-bromoethyl)-4,7-dimethyl-5-oxo-pyrazolo[3,4-c]isoquinolin-3-yl]piperidine-1-carboxylate (83.0 mg, 0.15 mmol) in 0.5 mL DMF was added 6-chloro-2-(1-methylpyrazol-4-yl)pyridin-3-amine (38.7 mg, 0.19 mmol) The resulting solution was heated to 60° C. for 3 h. Upon cooling down to r.t., the mixture was concentrated under reduced pressure. The residue was purified by silica gel column chromatography, eluted with ethyl acetate in dichloromethane (0 to 100%) to afford the desired product. LCMS calculated for C₃₆H₃₈ClN₈O₃ (M+H)⁺ m/z=665.3 Found 665.3.

### Step 3: 9-[1-[[6-chloro-2-(1-methylpyrazol-4-yl)-3-pyridyl]amino]ethyl]-4,7-dimethyl-3-(4-piperidyl)pyrazolo[3,4-c]isoquinolin-5-one

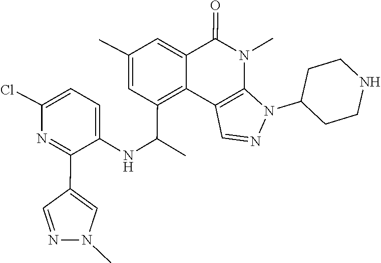 [SMILES: Cc1cc(C(C)Nc2ccc(Cl)nc2-c2cnn(C)c2)c2c(c1)c(=O)n(C)c1c2cnn1C1CCNCC1]

At 0° C., to a 2 dram vial containing benzyl 4-[9-[1-[[6-chloro-2-(1-methylpyrazol-4-yl)-3-pyridyl]amino]ethyl]-4,7-dimethyl-5-oxo-pyrazolo[3,4-c]isoquinolin-3-yl]piperidine-1-carboxylate (18.0 mg, 0.03 mmol) was added 0.5 mL hydrogen bromide solution (33 wt. % in acetic acid). The resulting solution was stirred at the same temperature for 30 min, then diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₃₂ClN₈O (M+H)⁺ m/z=531.2; found 531.2.

### Step 4: 9-(1-((6-chloro-2-(1-methyl-1H-pyrazol-4-yl)pyridin-3-yl)amino)ethyl)-4,7-dimethyl-3-(1-methylpiperidin-4-yl)-3,4-dihydro-5H-pyrazolo[3,4-c]isoquinolin-5-one

To a solution of 9-[1-[[6-chloro-2-(1-methylpyrazol-4-yl)-3-pyridyl]amino]ethyl]-4,7-dimethyl-3-(4-piperidyl)pyrazolo[3,4-c]isoquinolin-5-one (5.0 mg, 2.8 μmol) in 1 mL DCM was added formaldehyde (1.05 μL, 14.10 μmol), followed by sodium triacetoxyborohydride (1.8 mg, 8.46 μmol). The resulting solution was stirred at r.t. for 1 h, then concentrated under vacuum. The crude product was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₄ClN₈O (M+H)⁺ m/z=545.3, found 545.2.

### Examples 159-171

Examples 159-171 in Table 24 were prepared similarly as described for Example 158.

TABLE 24

| Ex- | | | LCMS |
|---|---|---|---|
| ample | | | m/z |
| # | Chemical name | Structure | (M + H)⁺ |
| 159 | 9-[1-[[6-chloro-2-(1-methyl-1,2,4- triazol-3-yl)-3-pyridyl]amino]ethyl]- 4,7-dimethyl-3-(1-methyl-4- piperidyl)pyrazolo[3,4-c]isoquinolin- 5-one | 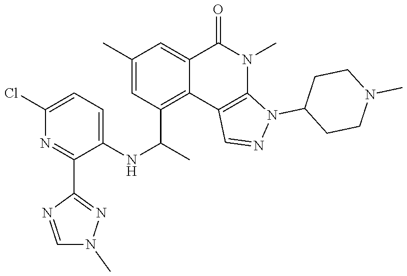 [SMILES: Cc1cc(C(C)Nc2ccc(Cl)nc2-c2ncn(C)n2)c2c(c1)c(=O)n(C)c1c2cnn1C1CCN(C)CC1] | 546.3 |
| 160 | 9-[1-[[6-chloro-2-(1-ethylpyrazol-4- yl)-3-pyridyl]amino]ethyl]-4,7- dimethyl-3-(1-methyl-4- piperidyl)pyrazolo[3,4-c]isoquinolin- 5-one | 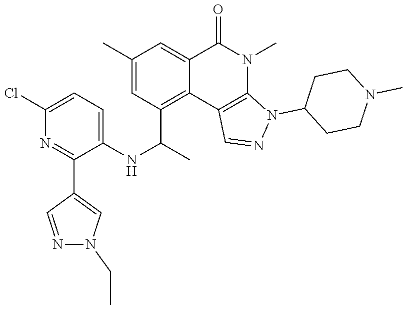 [SMILES: CCn1cc(-c2nc(Cl)ccc2NC(C)c2cc(C)cc3c(=O)n(C)c4c(cnn4C4CCN(C)CC4)c23)cn1] | 559.3 |
| 161 | 9-[1-[[6-chloro-2-(2-methyltetrazol- 5-yl)-3-pyridyl]amino]ethyl]-4,7- dimethyl-3-(1-methyl-4- piperidyl)pyrazolo[3,4-c]isoquinolin- 5-one | 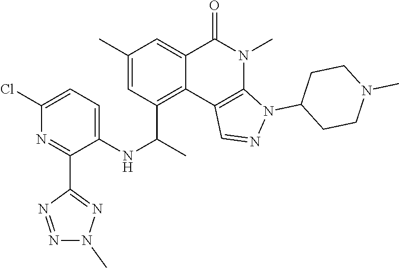 [SMILES: Cc1cc(C(C)Nc2ccc(Cl)nc2-c2nnn(C)n2)c2c(c1)c(=O)n(C)c1c2cnn1C1CCN(C)CC1] | 547.2 |
| 162 | 9-[1-[[6-chloro-2-(1,3,4-oxadiazol-2- yl)-3-pyridyl]amino]ethyl]-4,7- dimethyl-3-(1-methyl-4- piperidyl)pyrazolo[3,4-c]isoquinolin- 5-one | 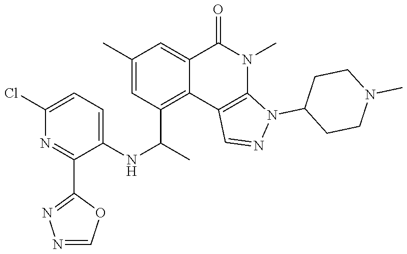 [SMILES: Cc1cc(C(C)Nc2ccc(Cl)nc2-c2nnco2)c2c(c1)c(=O)n(C)c1c2cnn1C1CCN(C)CC1] | 533.2 |
| 163 | 5-[6-chloro-3-[1-[4,7-dimethyl-3-(1- methyl-4-piperidyl)-5-oxo- pyrazolo[3,4-c]isoquinolin-9- yl]ethylamino]-2-pyridyl]-N-methyl- pyridine-2-carboxamide | 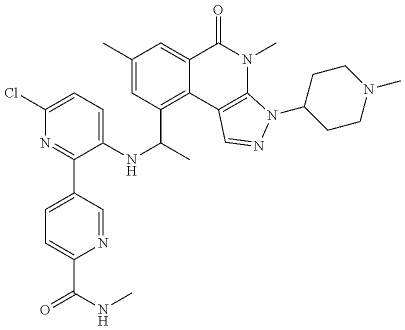 [SMILES: CNC(=O)c1ccc(-c2nc(Cl)ccc2NC(C)c2cc(C)cc3c(=O)n(C)c4c(cnn4C4CCN(C)CC4)c23)cn1] | 599.3 |
| 164 | 3-[6-chloro-3-[1-[4,7-dimethyl-3-(1- methyl-4-piperidyl)-5-oxo- pyrazolo[3,4-c]isoquinolin-9- yl]ethylamino]-2-pyridyl]-4H-1,2,4- oxadiazol-5-one | 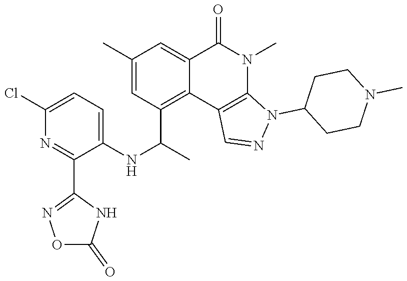 [SMILES: Cc1cc(C(C)Nc2ccc(Cl)nc2-c2noc(=O)[nH]2)c2c(c1)c(=O)n(C)c1c2cnn1C1CCN(C)CC1] | 549.2 |
| 165 | 9-[1-[[6-chloro-2-(1,3- dimethylpyrazol-4-yl)-3- pyridyl]amino]ethyl]-4,7-dimethyl-3- (1-methyl-4-piperidyl)pyrazolo[3,4- c]isoquinolin-5-one | 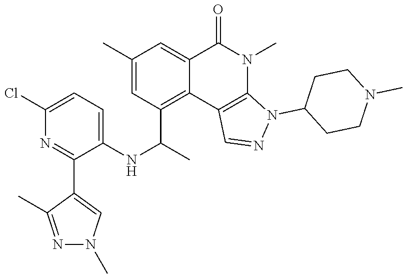 [SMILES: Cc1cc(C(C)Nc2ccc(Cl)nc2-c2cn(C)nc2C)c2c(c1)c(=O)n(C)c1c2cnn1C1CCN(C)CC1] | 559.3 |
| 166 | 9-[1-[[6-chloro-2-(1-methyl-6-oxo-3- pyridyl)-3-pyridyl]amino]ethyl]-4,7- dimethyl-3-(1-methyl-4- piperidyl)pyrazolo[3,4-c]isoquinolin- 5-one | 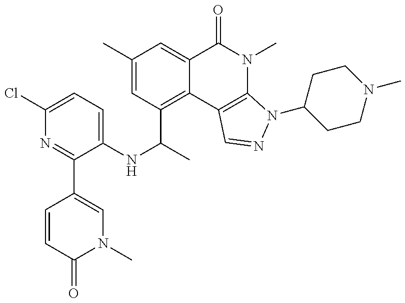 [SMILES: Cc1cc(C(C)Nc2ccc(Cl)nc2-c2ccc(=O)n(C)c2)c2c(c1)c(=O)n(C)c1c2cnn1C1CCN(C)CC1] | 572.3 |
| 167 | 9-[1-[[6-chloro-2-(4-hydroxy-1- piperidyl)-3-pyridyl]amino]ethyl]- 4,7-dimethyl-3-(1-methyl-4- piperidyl)pyrazolo[3,4-c]isoquinolin- 5-one | 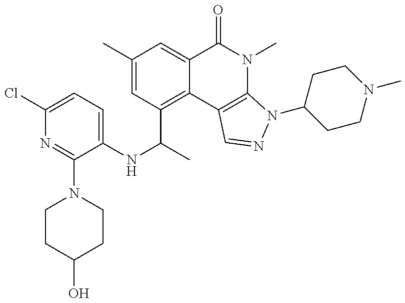 [SMILES: Cc1cc(C(C)Nc2ccc(Cl)nc2N2CCC(O)CC2)c2c(c1)c(=O)n(C)c1c2cnn1C1CCN(C)CC1] | 564.3 |
| 168 | 9-[1-[[6-chloro-2-(3-fluoro-4- pyridyl)-3-pyridyl]amino]ethyl]-4,7- dimethyl-3-(1-methyl-4- piperidyl)pyrazolo[3,4-c]isoquinolin- 5-one | 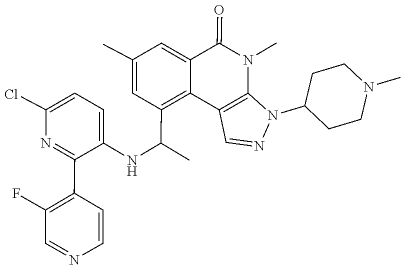 [SMILES: Cc1cc(C(C)Nc2ccc(Cl)nc2-c2ccncc2F)c2c(c1)c(=O)n(C)c1c2cnn1C1CCN(C)CC1] | 560.2 |
| 169 | 9-[1-[[6-chloro-2-(3-pyridyl)-3- pyridyl]amino]ethyl]-4,7-dimethyl-3- (1-methyl-4-piperidyl)pyrazolo[3,4- c]isoquinolin-5-one | 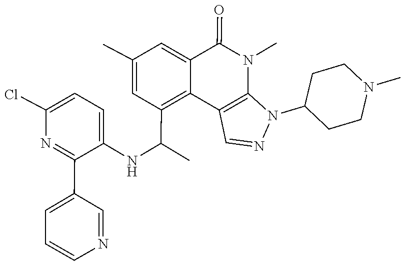 [SMILES: Cc1cc(C(C)Nc2ccc(Cl)nc2-c2cccnc2)c2c(c1)c(=O)n(C)c1c2cnn1C1CCN(C)CC1] | 542.2 |
| 170 | 5-[3-[1-[4,7-dimethyl-3-(1-methyl-4- piperidyl)-5-oxo-pyrazolo[3,4- c]isoquinolin-9-ylethylamino]-6- methyl-2-pyridyl]-N-methyl- pyridine-2-carboxamide | 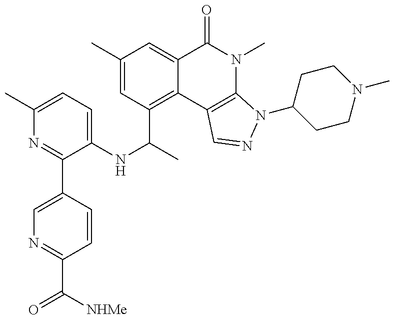 | 579.3 |
| 171 | 9-[1-[[6-chloro-2-(1-methyl-2-oxo-4- pyridyl)-3-pyridyl]amino]ethyl]-4,7- dimethyl-3-(1-methyl-4- piperidyl)pyrazolo[3,4-c]isoquinolin- 5-one | 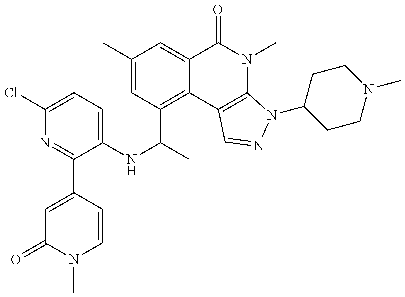 [SMILES: Cc1cc(C(C)Nc2ccc(Cl)nc2-c2ccn(C)c(=O)c2)c2c(c1)c(=O)n(C)c1c2cnn1C1CCN(C)CC1] | 572.3 |

### Example 172: 9-[1-[[6-Chloro-2-(1-methylpyrazol-4-yl)-3-pyridyl]amino]ethyl]-3-[1-(2-hydroxy-2-methyl-propyl)-4-piperidyl]-4,7-dimethyl-pyrazolo[3,4-c]isoquinolin-5-one

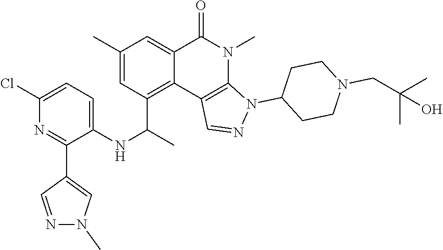 [SMILES: Cc1cc(C(C)Nc2ccc(Cl)nc2-c2cnn(C)c2)c2c(c1)c(=O)n(C)c1c2cnn1C1CCN(CC(C)(C)O)CC1]

To a solution of 9-[1-[[6-chloro-2-(1-methylpyrazol-4-yl)-3-pyridyl]amino]ethyl]-4,7-dimethyl-3-(4-piperidyl)pyrazolo[3,4-c]isoquinolin-5-one (Example 158, step 3) (5.0 mg, 0.01 mmol) in 0.5 mL EtOH was added 2,2-dimethyloxirane (8 μL, 0.09 mmol), the resulting solution was heated to 80° C. for 30 min. Upon cooling down to r.t. The mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₂H₄₀ClN₈O₂ (M+H)⁺ m/z=603.3; found 603.2.

### Examples 173-175

Examples 173-175 in Table 25 were prepared similarly as described for Example 172.

TABLE 25

| Ex- | | | LCMS |
|---|---|---|---|
| ample | | | m/z |
| # | Chemical name | Structure | (M + H)⁺ |
| 173 | 9-[1-[[6-chloro-2-(1- methylpyrazol-4-yl)-3- pyridyl]amino]ethyl]-3-[1- [(2R)-2-hydroxypropyl]-4- piperidyl]-4,7-dimethyl- pyrazolo[3,4-c]isoquinolin-5- one | 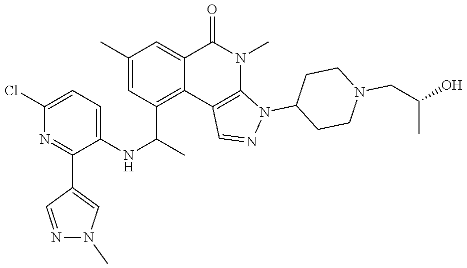 [SMILES: Cc1cc(C(C)Nc2ccc(Cl)nc2-c2cnn(C)c2)c2c(c1)c(=O)n(C)c1c2cnn1C1CCN(C[C@@H](C)O)CC1] | 589.3 |
| 174 | 9-[1-[[6-chloro-2-(1- methylpyrazol-4-yl)-3- pyridyl]amino]ethyl]-3-[1- [(2S)-2-hydroxypropyl]-4- piperidyl]-4,7-dimethyl- pyrazolo[3,4-c]isoquinolin-5- one | 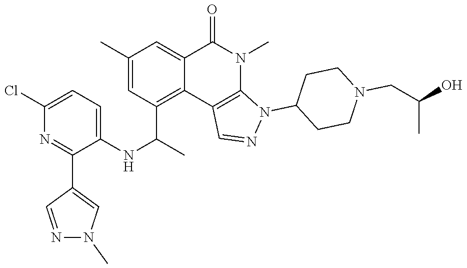 [SMILES: Cc1cc(C(C)Nc2ccc(Cl)nc2-c2cnn(C)c2)c2c(c1)c(=O)n(C)c1c2cnn1C1CCN(C[C@H](C)O)CC1] | 589.3 |
| 175 | 9-(1-((6-chloro-2-(1-methyl- 1H-1,2,4-triazol-3-yl)pyridin- 3-yl)amino)ethyl)-3-(1-(2- hydroxy-2- methylpropyl)piperidin-4-yl)- 4,7-dimethyl-3,4-dihydro-5H- pyrazolo[3,4-c]isoquinolin-5- one | 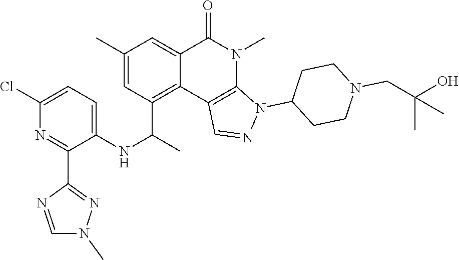 [SMILES: Cc1cc(C(C)Nc2ccc(Cl)nc2-c2ncn(C)n2)c2c(c1)c(=O)n(C)c1c2cnn1C1CCN(CC(C)(C)O)CC1] | 604.2 |

### Example 176: 9-(1-((6-Chloro-2-(1-methyl-1H-1,2,4-triazol-3-yl)pyridin-3-yl)amino)ethyl)-4,7-dimethyl-3-(piperidin-4-yl)-3,4-dihydro-5H-pyrazolo[3,4-c]isoquinolin-5-one

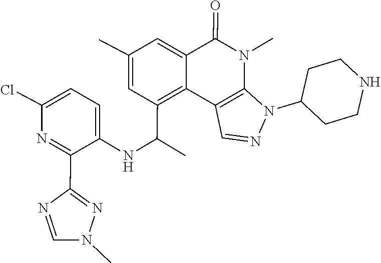 [SMILES: Cc1cc(C(C)Nc2ccc(Cl)nc2-c2ncn(C)n2)c2c(c1)c(=O)n(C)c1c2cnn1C1CCNCC1]

The titled compound was prepared using similar procedures as described for Example 158 with 6-chloro-2-(1-methyl-1H-1,2,4-triazol-3-yl)pyridin-3-amine replacing 6-chloro-2-(1-methyl-1H-pyrazol-4-yl)pyridin-3-amine in Step 2. The crude product was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₁ClN₉O (M+H)⁺ m/z=532.2; found 532.2.

### Example 177:9-[1-[[6-Chloro-2-(1-methyl-1,2,4-triazol-3-yl)-3-pyridyl]amino]ethyl]-3-[1-(2-hydroxyethyl)-4-piperidyl]-4,7-dimethyl-pyrazolo[3,4-c]isoquinolin-5-one

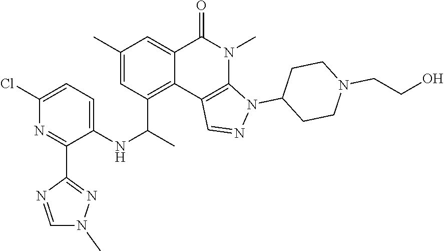 [SMILES: Cc1cc(C(C)Nc2ccc(Cl)nc2-c2ncn(C)n2)c2c(c1)c(=O)n(C)c1c2cnn1C1CCN(CCO)CC1]

To a solution of 9-[1-[[6-chloro-2-(1-methyl-1,2,4-triazol-3-yl)-3-pyridyl]amino]ethyl]-4,7-dimethyl-3-(4-piperidyl)pyrazolo[3,4-c]isoquinolin-5-one (5.0 mg, 0.01 mmol) in 1 ml DMF was added was added 2-[tert-butyl(dimethyl) silyl]oxyacetaldehyde (4.9 mg, 0.03 mmol), followed by sodium triacetoxyborohydride (5 mg, 0.023 mmol). The mixture was stirred for 30 min, solvent was removed under vacuum. 4 M HCl in dioxane (0.3 mL) and MeOH (0.2 mL) was added and the mixture was stirred for 10 min before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₅ClN₉O₂ (M+H)⁺ m/z=576.3; found page 576.2.

### Examples 178-179

Examples 178-179 in Table 26 were prepared similarly as described for Example 177.

TABLE 26

| Ex- | | | LCMS |
|---|---|---|---|
| ample | | | m/z |
| # | Chemical name | Structure | (M + H)⁺ |
| 178 | 9-(1-((6-chloro-2-(1-methyl- 1H-1,2,4-triazol-3- yl)pyridin-3-yl)amino)ethyl)- 4,7-dimethyl-3-(1-((1- methyl-1H-imidazol-2- yl)methyl)piperidin-4-yl)- 3,4-dihydro-5H- pyrazolo[3,4-c]isoquinolin-5- one | 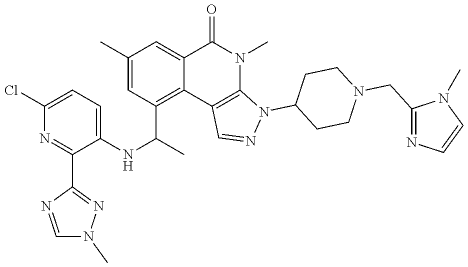 [SMILES: Cc1cc(C(C)Nc2ccc(Cl)nc2-c2ncn(C)n2)c2c(c1)c(=O)n(C)c1c2cnn1C1CCN(Cc2nccn2C)CC1] | 626.3 |
| 179 | 9-(1-((6-chloro-2-(1-methyl- 1H-1,2,4-triazol-3- yl)pyridin-3-yl)amino)ethyl)- 4,7-dimethyl-3-(1-(oxetan-3- yl)piperidin-4-yl)-3,4- dihydro-5H-pyrazolo[3,4- c]isoquinolin-5-one | 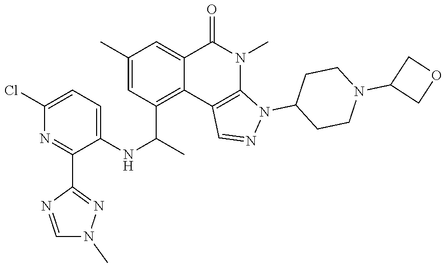 [SMILES: Cc1cc(C(C)Nc2ccc(Cl)nc2-c2ncn(C)n2)c2c(c1)c(=O)n(C)c1c2cnn1C1CCN(C2COC2)CC1] | 588.3 |

### Example 180: 3-[4-[9-[1-[[6-Chloro-2-(1-methyl-1,2,4-triazol-3-yl)-3-pyridyl]amino]ethyl]-4,7-dimethyl-5-oxo-pyrazolo[3,4-c]isoquinolin-3-yl]-1-piperidyl]propanenitrile

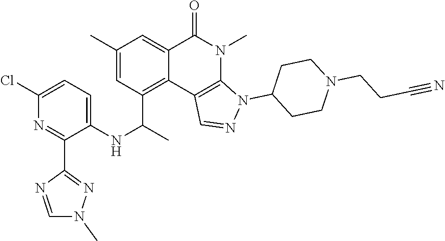 [SMILES: Cc1cc(C(C)Nc2ccc(Cl)nc2-c2ncn(C)n2)c2c(c1)c(=O)n(C)c1c2cnn1C1CCN(CCC#N)CC1]

The titled compound was prepared using similar procedures as described for Example 152 with 9-(1-((6-chloro-2-(1-methyl-1H-1,2,4-triazol-3-yl)pyridin-3-yl)amino)ethyl)-4,7-dimethyl-3-(piperidin-4-yl)-3,4-dihydro-5H-pyrazolo[3,4-c]isoquinolin-5-one replacing 9-(1-((6-chloro-2-(1-methyl-1H-pyrazol-4-yl)pyridin-3-yl)amino)ethyl)-7-methyl-4-(methyl-d3)-3-(pyrrolidin-3-yl)-3,4-dihydro-5H-pyrazolo[3,4-c]isoquinolin-5-one in step 2. The crude product was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₀H₃₄ClN₁₀O (M+H)⁺ m/z=585.2; found 585.2.

### Example 181: 9-(1-((6-Chloro-2-(1-methyl-1H-1,2,4-triazol-3-yl)pyridin-3-yl)amino)ethyl)-3-(1-(2,2-difluoroethyl)piperidin-4-yl)-4,7-dimethyl-3,4-dihydro-5H-pyrazolo[3,4-c]isoquinolin-5-one

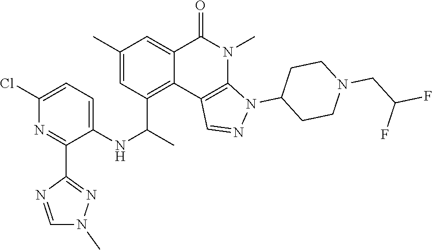 [SMILES: Cc1cc(C(C)Nc2ccc(Cl)nc2-c2ncn(C)n2)c2c(c1)c(=O)n(C)c1c2cnn1C1CCN(CC(F)F)CC1]

The titled compound was prepared using similar procedures as described for Example 142 with 9-(1-((6-chloro-2-(1-methyl-1H-1,2,4-triazol-3-yl)pyridin-3-yl)amino)ethyl)-4,7-dimethyl-3-(piperidin-4-yl)-3,4-dihydro-5H-pyrazolo[3,4-c]isoquinolin-5-one (Example 176) replacing 9-(1-((6-chloro-2-(1-methyl-1H-pyrazol-4-yl)pyridin-3-yl)amino)ethyl)-4,7-dimethyl-3-(pyrrolidin-3-yl)-3,4-dihydro-5H-pyrazolo[3,4-c]isoquinolin-5-one. The crude product was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₃ClF₂N₉O (M+H)⁺ m/z=596.2; found 596.2.

### Example 182: 3-(1-Acetylpiperidin-4-yl)-9-(1-((6-chloro-2-(1-methyl-1H-pyrazol-4-yl)pyridin-3-yl)amino)ethyl)-4,7-dimethyl-3,4-dihydro-5H-pyrazolo[3,4-c]isoquinolin-5-one

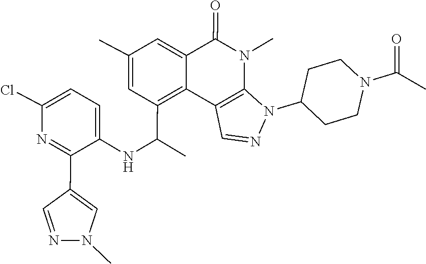 [SMILES: CC(=O)N1CCC(n2ncc3c4c(C(C)Nc5ccc(Cl)nc5-c5cnn(C)c5)cc(C)cc4c(=O)n(C)c32)CC1]

At 0° C., to a solution of 9-[1-[[6-chloro-2-(1-methylpyrazol-4-yl)-3-pyridyl]amino]ethyl]-4,7-dimethyl-3-(4-piperidyl)pyrazolo[3,4-c]isoquinolin-5-one (Example 158, step 3) (10.0 mg, 0.02 mmol) in 1 ml DCM was added N-ethyl-N-isopropyl-propan-2-amine (9 uL, 0.06 mmol). Then acetyl chloride (2 uL, 0.03 mmol) dissolved in 0.5 mL DCM was added dropwise. The resulting solution was stirred at 0° C. for 15 min and then concentrated under vacuum. The crude product was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₀H₃₄ClN₈O₂ (M+H)⁺ m/z=573.3; found 573.3.

### Examples 183-192

Examples 183-192 in Table 27 were prepared similarly as described for Example 182.

TABLE 27

| Ex- | | | LCMS |
|---|---|---|---|
| ample | | | m/z |
| # | Chemical name | Structure | (M + H)⁺ |
| 183 | 9-[1-[[6-chloro-2-(1- methylpyrazol-4-yl)-3- pyridyl]amino]ethyl]-4,7- dimethyl-3-(1- methylsulfonyl-4- piperidyl)pyrazolo[3,4- c]isoquinolin-5-one | 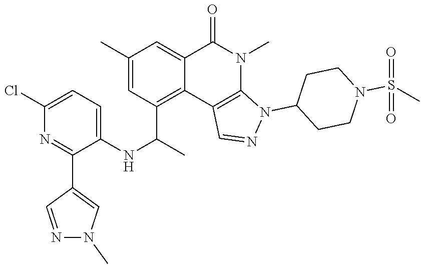 [SMILES: Cc1cc(C(C)Nc2ccc(Cl)nc2-c2cnn(C)c2)c2c(c1)c(=O)n(C)c1c2cnn1C1CCN(S(C)(=O)=O)CC1] | 609.2 |
| 184 | methyl 4-[9-[1-[[6-chloro- 2-(1-methylpyrazol-4-yl)-3- pyridyl]amino]ethyl]-4,7- dimethyl-5-oxo- pyrazolo[3,4-c]isoquinolin- 3-yl]piperidine-1- carboxylate | 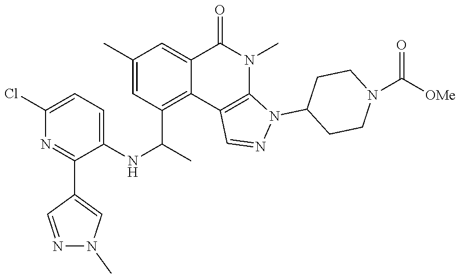 [SMILES: COC(=O)N1CCC(n2ncc3c4c(C(C)Nc5ccc(Cl)nc5-c5cnn(C)c5)cc(C)cc4c(=O)n(C)c32)CC1] | 589.2 |
| 185 | 9-[1-[[6-chloro-2-(1- methylpyrazol-4-yl)-3- pyridyl]amino]ethyl]-3-(1- cyclopropylsulfonyl-4- piperidyl)-4,7-dimethyl- pyrazolo[3,4-c]isoquinolin- 5-one | 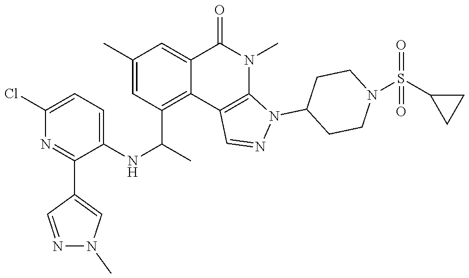 [SMILES: Cc1cc(C(C)Nc2ccc(Cl)nc2-c2cnn(C)c2)c2c(c1)c(=O)n(C)c1c2cnn1C1CCN(S(=O)(=O)C2CC2)CC1] | 635.2 |
| 186 | 4-(9-(1-((6-chloro-2-(1- methyl-1H-pyrazol-4- yl)pyridin-3- yl)amino)ethyl)-4,7- dimethyl-5-oxo-4,5- dihydro-3H-pyrazolo[3,4- c]isoquinolin-3-yl)-N,N- dimethylpiperidine-1- carboxamide | 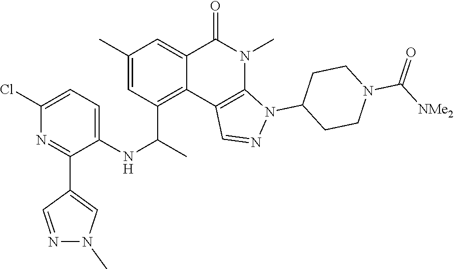 [SMILES: Cc1cc(C(C)Nc2ccc(Cl)nc2-c2cnn(C)c2)c2c(c1)c(=O)n(C)c1c2cnn1C1CCN(C(=O)N(C)C)CC1] | 602.3 |
| 187 | 3-(1-acetylpiperidin-4-yl)- 9-(1-((6-chloro-1′-methyl- 6′-oxo-1′,6′-dihydro-[2,3′- bipyridin]-3- yl)amino)ethyl)-4,7- dimethyl-3,4-dihydro-5H- pyrazolo[3,4-c]isoquinolin- 5-one | 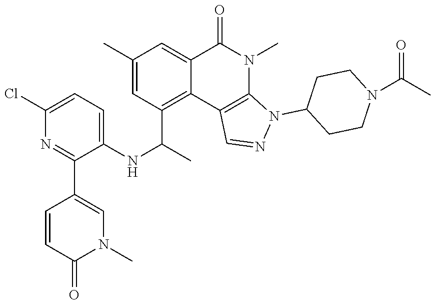 [SMILES: CC(=O)N1CCC(n2ncc3c4c(C(C)Nc5ccc(Cl)nc5-c5ccc(=O)n(C)c5)cc(C)cc4c(=O)n(C)c32)CC1] | 600.2 |
| 188 | 3-(1-acetylpiperidin-4-yl)- 9-(1-((6-chloro-2-(1- methyl-1H-1,2,4-triazol-3- yl)pyridin-3- yl)amino)ethyl)-4,7- dimethyl-3,4-dihydro-5H- pyrazolo[3,4-c]isoquinolin- 5-one | 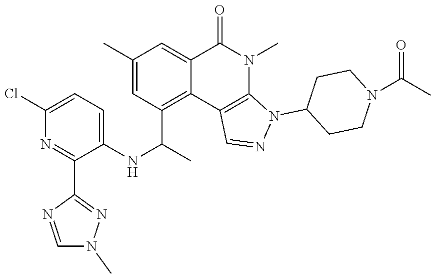 [SMILES: CC(=O)N1CCC(n2ncc3c4c(C(C)Nc5ccc(Cl)nc5-c5ncn(C)n5)cc(C)cc4c(=O)n(C)c32)CC1] | 574.2 |
| 189 | 4,7-dimethyl-9-(1-((6- methyl-2-(1-methyl-1H- pyrazol-4-yl)pyridin-3- yl)amino)ethyl)-3-(1- (methylsulfonyl)piperidin- 4-yl)-3,4-dihydro-5H- pyrazolo[3,4-c]isoquinolin- 5-one | 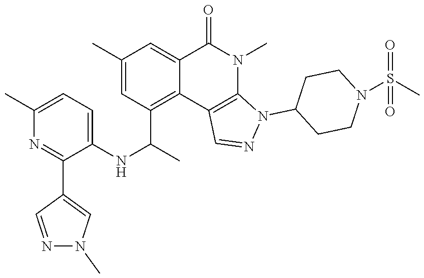 | 589.3 |
| 190 | 9-(1-((6-chloro-2-(1- methyl-1H-1,2,4-triazol-3- yl)pyridin-3- yl)amino)ethyl)-4,7- dimethyl-3-(1- (methylsulfonyl)piperidin- 4-yl)-3,4-dihydro-5H- pyrazolo[3,4-c]isoquinolin- 5-one | 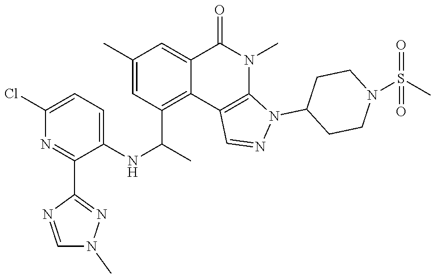 [SMILES: Cc1cc(C(C)Nc2ccc(Cl)nc2-c2ncn(C)n2)c2c(c1)c(=O)n(C)c1c2cnn1C1CCN(S(C)(=O)=O)CC1] | 610.2 |
| 191 | 9-(1-((6-chloro-2-(2- methyl-1-oxoisoindolin-5- yl)pyridin-3- yl)amino)ethyl)-4,7- dimethyl-3-(1- (methylsulfonyl)piperidin- 4-yl)-3,4-dihydro-5H- pyrazolo[3,4-c]isoquinolin- 5-one | 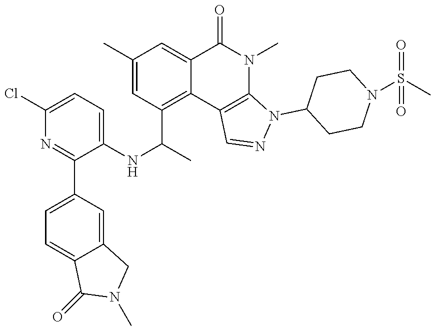 [SMILES: Cc1cc(C(C)Nc2ccc(Cl)nc2-c2ccc3c(c2)CN(C)C3=O)c2c(c1)c(=O)n(C)c1c2cnn1C1CCN(S(C)(=O)=O)CC1] | 674.2 |
| 192 | 9-(1-((6-chloro-2-(quinolin- 6-yl)pyridin-3- yl)amino)ethyl)-4,7- dimethyl-3-(1- (methylsulfonyl)piperidin- 4-yl)-3,4-dihydro-5H- pyrazolo[3,4-c]isoquinolin- 5-one | 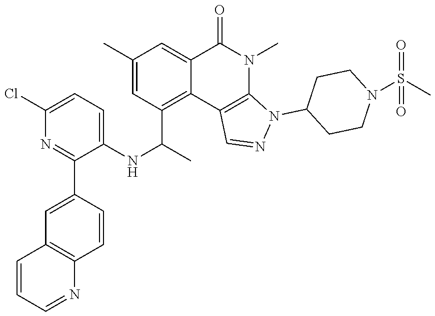 [SMILES: Cc1cc(C(C)Nc2ccc(Cl)nc2-c2ccc3ncccc3c2)c2c(c1)c(=O)n(C)c1c2cnn1C1CCN(S(C)(=O)=O)CC1] | 656.2 |

### Example 193: 9-[1-[[6-Chloro-2-(1-methylpyrazol-4-yl)-3-pyridyl]amino]ethyl]-3-[1-(2-hydroxyacetyl)-4-piperidyl]-4,7-dimethyl-pyrazolo[3,4-c]isoquinolin-5-one

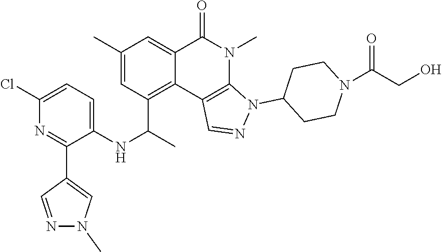 [SMILES: Cc1cc(C(C)Nc2ccc(Cl)nc2-c2cnn(C)c2)c2c(c1)c(=O)n(C)c1c2cnn1C1CCN(C(=O)CO)CC1]

To a solution of 9-(1-((6-chloro-2-(1-methyl-1H-pyrazol-4-yl)pyridin-3-yl)amino)ethyl)-4,7-dimethyl-3-(piperidin-4-yl)-3,4-dihydro-5H-pyrazolo[3,4-c]isoquinolin-5-one (Example 158, step 3) (10 mg, 0.02 mmol) in DMF (0.2 mL) was added 2-hydroxyacetic acid (3 mg, 0.04 mmol), HATU (15 mg, 0.04 mmol) and N,N-diisopropylethylamine (7 mg, 0.06). The reaction mixture was stirred at room temperature for 30 minutes. The resulting mixture was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₀H₃₄ClN₈O₃ (M+H)⁺ m/z=589.2; found 589.2.

### Examples 194-197

Examples 194-197 in Table 28 were prepared similarly as described for Example 193.

TABLE 28

| Ex- | | | LCMS |
|---|---|---|---|
| ample | | | m/z |
| # | Chemical Name | Structure | (M + H)⁺ |
| 194 | 1-(4-(9-(1-((6-chloro-2-(1- methyl-1H-1,2,4-triazol-3- yl)pyridin-3-yl)amino)ethyl)- 4,7-dimethyl-5-oxo-4,5- dihydro-3H-pyrazolo[3,4- c]isoquinolin-3- yl)piperidine-1- carbonyl)cyclopropane-1- carbonitrile | 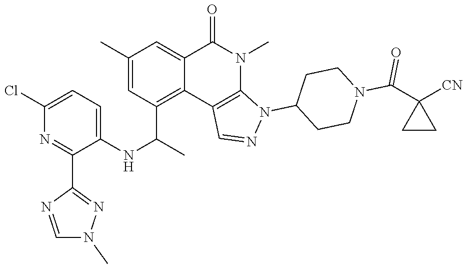 [SMILES: Cc1cc(C(C)Nc2ccc(Cl)nc2-c2ncn(C)n2)c2c(c1)c(=O)n(C)c1c2cnn1C1CCN(C(=O)C2(C#N)CC2)CC1] | 625.3 |
| 195 | 9-(1-((6-chloro-2-(1-methyl- 1H-1,2,4-triazol-3- yl)pyridin-3-yl)amino)ethyl)- 4,7-dimethyl-3-(1-(1-methyl- 1H-pyrazole-5- carbonyl)piperidin-4-yl)-3,4- dihydro-5H-pyrazolo[3,4- c]isoquinolin-5-one | 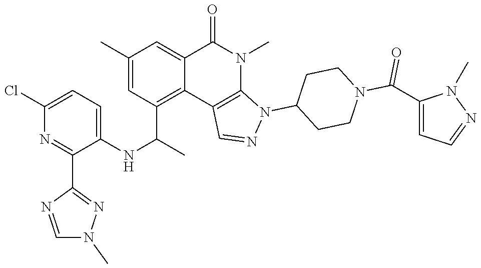 [SMILES: Cc1cc(C(C)Nc2ccc(Cl)nc2-c2ncn(C)n2)c2c(c1)c(=O)n(C)c1c2cnn1C1CCN(C(=O)c2ccnn2C)CC1] | 640.3 |
| 196 | 9-(1-((6-chloro-2-(1-methyl- 1H-pyrazol-4-yl)pyridin-3- yl)amino)ethyl)-3-(1- (dimethylglycyl)piperidin-4- yl)-4,7-dimethyl-3,4- dihydro-5H-pyrazolo[3,4- c]isoquinolin-5-one | 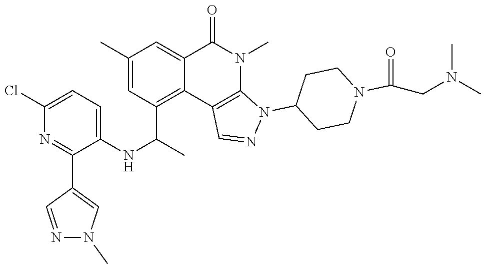 [SMILES: Cc1cc(C(C)Nc2ccc(Cl)nc2-c2cnn(C)c2)c2c(c1)c(=O)n(C)c1c2cnn1C1CCN(C(=O)CN(C)C)CC1] | 616.3 |
| 197 | 9-(1-((6-chloro-2-(1-methyl- 1H-1,2,4-triazol-3- yl)pyridin-3-yl)amino)ethyl)- 4,7-dimethyl-3-(1-(methyl-L- prolyl)piperidin-4-yl)-3,4- dihydro-5H-pyrazolo[3,4- c]isoquinolin-5-one | 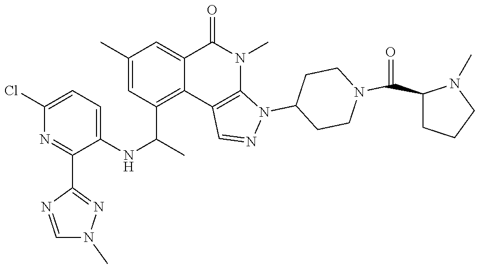 [SMILES: Cc1cc(C(C)Nc2ccc(Cl)nc2-c2ncn(C)n2)c2c(c1)c(=O)n(C)c1c2cnn1C1CCN(C(=O)[C@@H]2CCCN2C)CC1] | 643.3 |

### Example 198: 9-(1-((6-Chloro-2-(1-methyl-1H-pyrazol-4-yl)pyridin-3-yl)amino)ethyl)-4-methyl-3-(1-methylpiperidin-4-yl)-5-oxo-4,5-dihydro-3H-pyrazolo[3,4-c]isoquinoline-7-carbonitrile

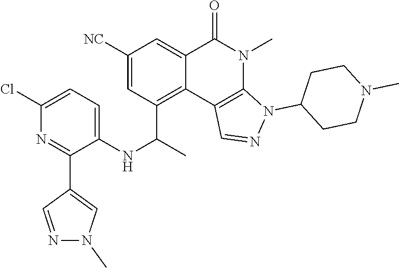 [SMILES: CC(Nc1ccc(Cl)nc1-c1cnn(C)c1)c1cc(C#N)cc2c(=O)n(C)c3c(cnn3C3CCN(C)CC3)c12]

### Step 1: tert-butyl 4-(9-acetyl-7-chloro-4-methyl-5-oxo-pyrazolo[3,4-c]isoquinolin-3-yl)piperidine-1-carboxylate

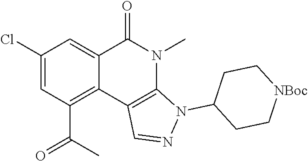 [SMILES: CC(=O)c1cc(Cl)cc2c(=O)n(C)c3c(cnn3C3CCN(C(=O)OC(C)(C)C)CC3)c12]

The titled compound was prepared using similar procedures as described for Example 81 with tert-butyl 4-hydrazineylpiperidine-1-carboxylate replacing tert-butyl 3-hydrazineylazetidine-1-carboxylate in Step 2, and 3-bromo-5-chloro-2-iodobenzoic acid replacing 3-bromo-2-iodo-5-methylbenzoic acid in step 4. The resulting residue was purified by silica gel column chromatography, eluted with ethyl acetate in DCM (0 to 100%) to afford the desired product as a white solid. LCMS calculated for C₂₃H₂₈ClN₄O₄ (M+H)⁺ m/z=459.2, found 459.2.

### Step 2: tert-butyl 4-(9-acetyl-7-cyano-4-methyl-5-oxo-pyrazolo[3,4-c]isoquinolin-3-yl)piperidine-1-carboxylate

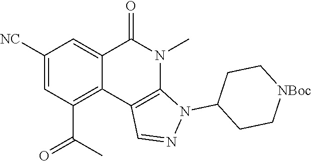 [SMILES: CC(=O)c1cc(C#N)cc2c(=O)n(C)c3c(cnn3C3CCN(C(=O)OC(C)(C)C)CC3)c12]

To a screw-cap vial equipped with a magnetic stir bar was added tert-butyl 4-(9-acetyl-7-chloro-4-methyl-5-oxo-pyrazolo[3,4-c]isoquinolin-3-yl)piperidine-1-carboxylate (100 mg, 0.22 mmol), tBuXPhos Pd G3 (35 mg, 0.04 mmol), zinc cyanide (13 mg, 0.11 mmol) and zinc formate dihydrate (63 mg, 0.33 mmol). The vial was sealed with a Teflon-lined septum, evacuated, and backfilled with nitrogen three times, then 4 mL DMAc was added. The resulting mixture was heated at 110° C. for 12 h. Upon cooling to room temperature, the mixture was diluted with 20 mL water and 20 mL EtOAc. The aqueous layer was extracted with EtOAc (3×10 mL). The combined organic layers were washed with brine (20 mL), dried over anhydrous sodium sulfate. After filtration, the filtrate was concentrated under reduced pressure. The resulting residue was purified by silica gel column chromatography, eluted with ethyl acetate in dichloromethane (0 to 100%) to afford the desired product. LCMS calculated for C₂₄H₂₈N₅O₄ (M+H)⁺ m/z=450.2; found 450.2.

### Step 3: tert-butyl 4-(7-cyano-9-(1-hydroxyethyl)-4-methyl-5-oxo-4,5-dihydro-3H-pyrazolo[3,4-c]isoquinolin-3-yl)piperidine-1-carboxylate

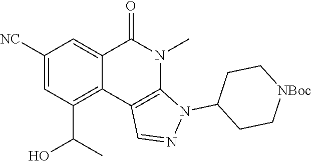 [SMILES: CC(O)c1cc(C#N)cc2c(=O)n(C)c3c(cnn3C3CCN(C(=O)OC(C)(C)C)CC3)c12]

At 0° C., to a solution of tert-butyl 4-(9-acetyl-7-cyano-4-methyl-5-oxo-pyrazolo[3,4-c]isoquinolin-3-yl)piperidine-1-carboxylate (80 mg, 0.18 mmol) in the mixture of 2 mL MeOH and 2 mL DCM was added sodium borohydride (10 mg, 0.27 mmol), the resulting solution was stirred at 0° C. for 30 min, then 1 ml 0.5 N HCl was added, the resulting solution was stirred at the same temperature for 10 min, then poured into the mixture of DCM and water. The aqueous layer was washed with DCM for 3 times. The combined organic layers were washed with brine, dried over anhydrous sodium sulfate. After filtration, the filtrate was concentrated under reduced pressure. The resulting residue was purified by silica gel column chromatography, eluted with ethyl acetate in dichloromethane (0 to 100%) to afford the desired product. LCMS calculated for C₂₄H₃₀N₅O₄ (M+H)⁺ m/z=452.2; found 452.2.

### Step 4: tert-butyl 4-[9-(1-bromoethyl)-7-cyano-4-methyl-5-oxo-pyrazolo[3,4-c]isoquinolin-3-yl]piperidine-1-carboxylate

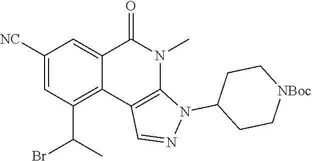 [SMILES: CC(Br)c1cc(C#N)cc2c(=O)n(C)c3c(cnn3C3CCN(C(=O)OC(C)(C)C)CC3)c12]

At 0° C., to a solution of tert-butyl 4-[7-cyano-9-(1-hydroxyethyl)-4-methyl-5-oxo-pyrazolo[3,4-c]isoquinolin-3-yl]piperidine-1-carboxylate (100 mg, 0.22 mmol) in 6 mL THF was added pyridine (0.07 mL, 0.89 mmol), then tribromophosphane (42 uL, 0.44 mmol) dissolved in 1 mL THF was added. The resulting solution was slowly warm to r.t. and stirred for 2 h. The resulting solution was diluted with DCM and water. The aqueous layer was washed with DCM for three times. The combined organic layers were washed with brine (20 mL), dried over anhydrous sodium sulfate. After filtration, the filtrate was concentrated under reduced pressure. The residue was used directly in the next step without further purification.

### Step 5: 9-[1-[[6-chloro-2-(1-methylpyrazol-4-yl)-3-pyridyl]amino]ethyl]-4-methyl-5-oxo-3-(4-piperidyl)pyrazolo[3,4-c]isoquinoline-7-carbonitrile

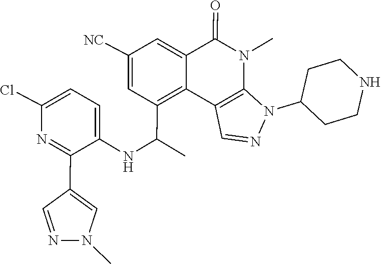 [SMILES: CC(Nc1ccc(Cl)nc1-c1cnn(C)c1)c1cc(C#N)cc2c(=O)n(C)c3c(cnn3C3CCNCC3)c12]

tert-Butyl 4-[9-(1-bromoethyl)-7-cyano-4-methyl-5-oxo-pyrazolo[3,4-c]isoquinolin-3-yl]piperidine-1-carboxylate (74 mg, 0.14 mmol) in 2 dram vial was added DMF (0.5 mL), followed by 6-chloro-2-(1-methylpyrazol-4-yl)pyridin-3-amine (60 mg, 0.29 mmol) The mixture was heated to 60° C. for 3 h. Upon cooling to room temperature, 0.5 mL TFA was added. The resulting solution was stirred for 30 min before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₂₉ClN₉O (M+H)⁺ m/z=542.2; found 542.2.

### Step 6: 9-[1-[[6-chloro-2-(1-methylpyrazol-4-yl)-3-pyridyl]amino]ethyl]-4-methyl-3-(1-methyl-4-piperidyl)-5-oxo-pyrazolo[3,4-c]isoquinoline-7-carbonitrile

To a solution of 9-[1-[[6-chloro-2-(1-methylpyrazol-4-yl)-3-pyridyl]amino]ethyl]-4-methyl-5-oxo-3-(4-piperidyl)pyrazolo[3,4-c]isoquinoline-7-carbonitrile (10 mg, 0.02 mmol) in 1 mL DCM was added formaldehyde (1 uL, 14 umol), followed by sodium triacetoxyborohydride (12 mg, 0.06 mmol). The resulting solution was stirred at r.t. for 1 h, then concentrated under vacuum. The crude product was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₁ClN₉O (M+H)⁺ m/z=556.2; found 556.2.

### Example 199: 9-(1-((6-Chloro-2-(1-methyl-1H-1,2,4-triazol-3-yl)pyridin-3-yl)amino)ethyl)-4-ethyl-7-methyl-2-(tetrahydro-2H-pyran-4-yl)-2,4-dihydro-5H-pyrazolo[3,4-c]isoquinolin-5-one

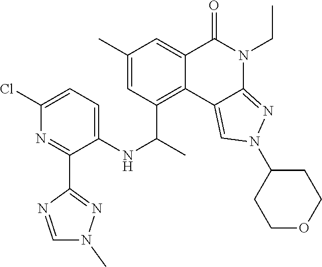 [SMILES: CCn1c(=O)c2cc(C)cc(C(C)Nc3ccc(Cl)nc3-c3ncn(C)n3)c2c2cn(C3CCOCC3)nc21]

### Step 1: 9-(1-((6-chloro-2-(1-methyl-1H-1,2,4-triazol-3-yl)pyridin-3-yl)amino)ethyl)-4-ethyl-3-(4-methoxybenzyl)-7-methyl-3,4-dihydro-5H-pyrazolo[3,4-c]isoquinolin-5-one

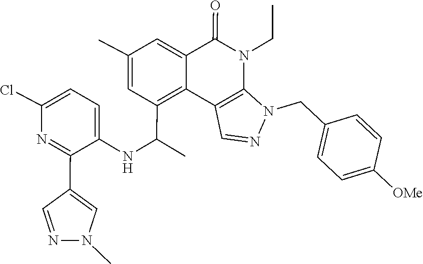 [SMILES: CCn1c(=O)c2cc(C)cc(C(C)Nc3ccc(Cl)nc3-c3cnn(C)c3)c2c2cnn(Cc3ccc(OC)cc3)c21]

The titled compound was prepared using similar procedures as described for Example 26 with 1-iodoethane replacing iodomethane in step 2 and 6-chloro-2-(1-methyl-1H-1,2,4-triazol-3-yl)pyridin-3-amine replacing methyl 2-aminobenzoate in Step 7. The resulting mixture was diluted with water and extracted with EtOAc. The combined organics were washed with sat. NaCl, dried with anhydrous sodium sulfate, filtered, and concentrated under reduced pressure. The crude product was used in the next step without purification. LCMS calculated for C₃₁H₃₂ClN₈O₂ (M+H)⁺ m/z=583.2; found 583.2.

### Step 2: 9-(1-((6-chloro-2-(1-methyl-1H-1,2,4-triazol-3-yl)pyridin-3-yl)amino)ethyl)-4-ethyl-7-methyl-2,4-dihydro-5H-pyrazolo[3,4-c]isoquinolin-5-one

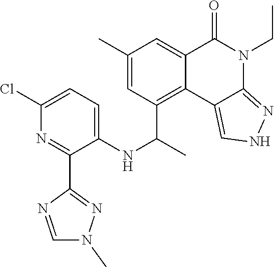 [SMILES: CCn1c(=O)c2cc(C)cc(C(C)Nc3ccc(Cl)nc3-c3ncn(C)n3)c2c2c[nH]nc21]

A solution of methyl 9-(1-((6-chloro-2-(1-methyl-1H-1,2,4-triazol-3-yl)pyridin-3-yl)amino)ethyl)-4-ethyl-3-(4-methoxybenzyl)-7-methyl-3,4-dihydro-5H-pyrazolo[3,4-c]isoquinolin-5-one (60 mg, 0.1 mmol) in TFA (2 ml) and DCM (2 mL) was stirred at 60° C. for 1 hour. The reaction mixture was concentrated under reduced pressure. The resulting solid was dissolved in sat. NaHCO₃ and extracted with dichloromethane (2×50 mL). The combined organics were washed with sat. NaCl and dried with anhydrous sodium sulfate, filtered, and concentrated under reduced pressure. The residue was purified by silica gel column chromatography, eluted with DCM and ethyl acetate to provide the desired product (26 mg, 56% yield). LCMS calculated for C₂₃H₂₄ClN₈O (M+H)⁺ m/z=463.2; found 463.2.

### Step 3: 9-(1-((6-chloro-2-(1-methyl-1H-1,2,4-triazol-3-yl)pyridin-3-yl)amino)ethyl)-4-ethyl-7-methyl-2-(tetrahydro-2H-pyran-4-yl)-2,4-dihydro-5H-pyrazolo[3,4-c]isoquinolin-5-one

To a solution of 9-(1-((6-chloro-2-(1-methyl-1H-1,2,4-triazol-3-yl)pyridin-3-yl)amino)ethyl)-4-ethyl-7-methyl-2,4-dihydro-5H-pyrazolo[3,4-c]isoquinolin-5-one (8 mg, 0.02 mmol) in DMF (0.2 mL) was added 4-iodotetrahydropyran (18.3 mg, 0.09 mmol) and potassium carbonate (11.9 mg, 0.09 mmol). The reaction mixture was stirred at 80° C. for 8 hours. The mixture was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₃₂ClN₈O₂ (M+H)⁺ m/z=547.2; found: 547.2;

### Example 200

Example 200 in Table 29 were prepared similarly as described for Example 199.

TABLE 29

| Ex- | | | LCMS |
|---|---|---|---|
| ample | | | m/z |
| # | Chemical Name | Structure | (M + H)⁺ |
| 200 | 3-(6-chloro-3-((1-(4,7-dimethyl-5- oxo-2-(tetrahydro-2H-pyran-4-yl)- 4,5-dihydro-2H-pyrazolo[3,4- c]isoquinolin-9- yl)ethyl)amino)pyridin-2-yl)-1,2,4- oxadiazol-5(4H)-one | 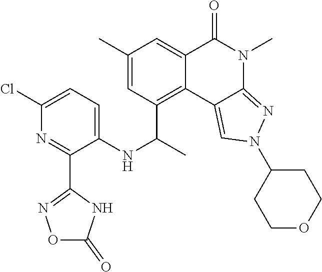 [SMILES: Cc1cc(C(C)Nc2ccc(Cl)nc2-c2noc(=O)[nH]2)c2c(c1)c(=O)n(C)c1nn(C3CCOCC3)cc21] | 536.2 |

### Example 201: 9-(1-((6-Chloro-2-(1-methyl-1H-1,2,4-triazol-3-yl)pyridin-3-yl)amino)ethyl)-4-ethyl-7-methyl-2-(1-methylpiperidin-4-yl)-2,4-dihydro-5H-pyrazolo[3,4-c]isoquinolin-5-one

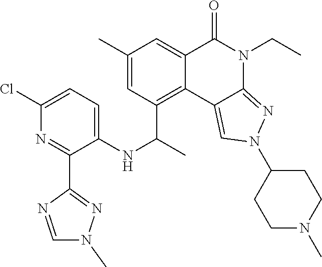 [SMILES: CCn1c(=O)c2cc(C)cc(C(C)Nc3ccc(Cl)nc3-c3ncn(C)n3)c2c2cn(C3CCN(C)CC3)nc21]

### Step 1: tert-butyl 4-(9-(1-((6-chloro-2-(1-methyl-1H-1,2,4-triazol-3-yl)pyridin-3-yl)amino)ethyl)-4-ethyl-7-methyl-5-oxo-4,5-dihydro-2H-pyrazolo[3,4-c]isoquinolin-2-yl)piperidine-1-carboxylate

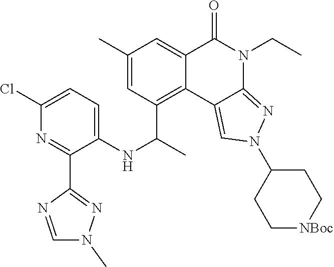 [SMILES: CCn1c(=O)c2cc(C)cc(C(C)Nc3ccc(Cl)nc3-c3ncn(C)n3)c2c2cn(C3CCN(C(=O)OC(C)(C)C)CC3)nc21]

The titled compound was prepared using similar procedures as described for Example 26 1-iodoethane replacing iodomethane in step 2, 6-chloro-2-(1-methyl-1H-1,2,4-triazol-3-yl)pyridin-3-amine replacing methyl 2-aminobenzoate in Step 7, and 1-boc-4-bromopiperidine replacing 1-iodopropane in step 9. The resulting mixture was diluted with water and extracted with EtOAc. The combined organics were washed with sat. NaCl, dried with anhydrous sodium sulfate, filtered, and concentrated under reduced pressure. The crude product was used in the next step without purification. LCMS calculated for C₃₃H₄₁ClN₉O₃ (M+H)⁺ m/z=646.3; found 646.3.

### Step 2: 9-(1-((6-chloro-2-(1-methyl-1H-1,2,4-triazol-3-yl)pyridin-3-yl)amino)ethyl)-4-ethyl-7-methyl-2-(piperidin-4-yl)-2,4-dihydro-5H-pyrazolo[3,4-c]isoquinolin-5-one

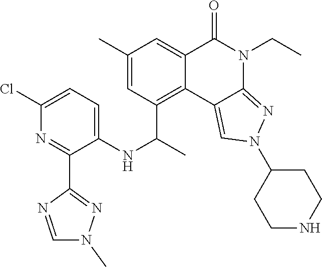 [SMILES: CCn1c(=O)c2cc(C)cc(C(C)Nc3ccc(Cl)nc3-c3ncn(C)n3)c2c2cn(C3CCNCC3)nc21]

A solution of tert-butyl 4-(9-(1-((6-chloro-2-(1-methyl-1H-1,2,4-triazol-3-yl)pyridin-3-yl)amino)ethyl)-4-ethyl-7-methyl-5-oxo-4,5-dihydro-2H-pyrazolo[3,4-c]isoquinolin-2-yl)piperidine-1-carboxylate (50 mg, 0.08 mmol) in 4N HCl in dioxane (2 mL) was stirred at room temperature for 30 min. The mixture diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid (22 mg, 51%). LCMS calculated for C₂₈H₃₃ClN₉O (M+H)⁺ m/z=546.2; found 546.2.

### Step 3: 9-(1-((6-chloro-2-(1-methyl-1H-1,2,4-triazol-3-yl)pyridin-3-yl)amino)ethyl)-4-ethyl-7-methyl-2-(1-methylpiperidin-4-yl)-2,4-dihydro-5H-pyrazolo[3,4-c]isoquinolin-5-one

To a mixture of 9-(1-((6-chloro-2-(1-methyl-1H-1,2,4-triazol-3-yl)pyridin-3-yl)amino)ethyl)-4-ethyl-7-methyl-2-(piperidin-4-yl)-2,4-dihydro-5H-pyrazolo[3,4-c]isoquinolin-5-one (5 mg, 0.01 mmol) was added DCM (0.5 mL), formaldehyde (water solution, 10 μL) and sodium triacetoxyborohydride (4 mg, 0.02 mmol). The mixture was stirred for 30 min, then diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₅ClN₉O (M+H)⁺ m/z=560.2; found 560.2.

### Example 202: 2-(1-Acetylpiperidin-4-yl)-9-(1-((6-chloro-2-(1-methyl-1H-1,2,4-triazol-3-yl)pyridin-3-yl)amino)ethyl)-4-ethyl-7-methyl-2,4-dihydro-5H-pyrazolo[3,4-c]isoquinolin-5-one

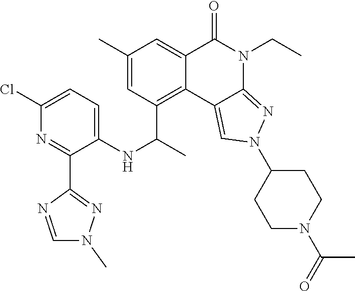 [SMILES: CCn1c(=O)c2cc(C)cc(C(C)Nc3ccc(Cl)nc3-c3ncn(C)n3)c2c2cn(C3CCN(C(C)=O)CC3)nc21]

To a solution of 9-(1-((6-chloro-2-(1-methyl-1H-1,2,4-triazol-3-yl)pyridin-3-yl)amino)ethyl)-4-ethyl-7-methyl-2-(piperidin-4-yl)-2,4-dihydro-5H-pyrazolo[3,4-c]isoquinolin-5-one (8 mg, 0.01 mmol) in DCM (0.2 mL) was added acetyl chloride (1.73 mg, 0.015 mmol) and N,N-diisopropylethylamine (2.84 mg, 0.015 mmol). The mixture was stirred for 30 min, then diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₀H₃₅ClN₉O₂ (M+H)⁺ m/z=588.2; found 588.2.

### Example 203: 9-(1-((6-Chloro-2-(1-methyl-1H-1,2,4-triazol-3-yl)pyridin-3-yl)amino)ethyl)-4-ethyl-2-(1-(2-hydroxyethyl)piperidin-4-yl)-7-methyl-2,4-dihydro-5H-pyrazolo[3,4-c]isoquinolin-5-one

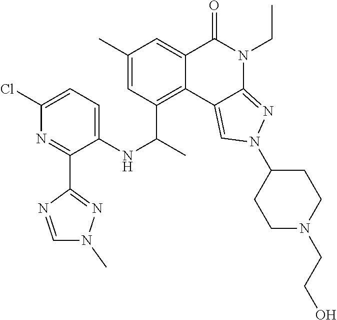 [SMILES: CCn1c(=O)c2cc(C)cc(C(C)Nc3ccc(Cl)nc3-c3ncn(C)n3)c2c2cn(C3CCN(CCO)CC3)nc21]

To a mixture of 9-(1-((6-chloro-2-(1-methyl-1H-1,2,4-triazol-3-yl)pyridin-3-yl)amino)ethyl)-4-ethyl-7-methyl-2-(piperidin-4-yl)-2,4-dihydro-5H-pyrazolo[3,4-c]isoquinolin-5-one (10 mg, 0.02 mmol) was added DCM (0.5 mL), 2-((tert-butyldimethylsilyl)oxy) acetaldehyde (16 mg, 0.09 mmol) and sodium triacetoxyborohydride (11.6 mg, 0.05 mmol). The mixture was stirred for 30 min, solvent was removed under vacuum. 4 M HCl in dioxane (0.3 mL) and MeOH (0.2 mL) was added and the mixture was stirred for 10 min before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₀H₃₇ClN₉O₂ (M+H)⁺ m/z=590.3; found 590.3.

### Example 204: 3-(4-(9-(1-((6-Chloro-2-(1-methyl-1H-1,2,4-triazol-3-yl)pyridin-3-yl)amino)ethyl)-4-ethyl-7-methyl-5-oxo-4,5-dihydro-2H-pyrazolo[3,4-c]isoquinolin-2-yl)piperidin-1-yl)propanenitrile

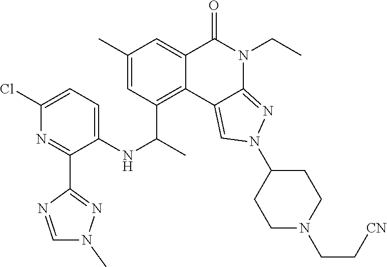 [SMILES: CCn1c(=O)c2cc(C)cc(C(C)Nc3ccc(Cl)nc3-c3ncn(C)n3)c2c2cn(C3CCN(CCC#N)CC3)nc21]

To a solution 9-(1-((6-chloro-2-(1-methyl-1H-1,2,4-triazol-3-yl)pyridin-3-yl)amino)ethyl)-4-ethyl-7-methyl-2-(piperidin-4-yl)-2,4-dihydro-5H-pyrazolo[3,4-c]isoquinolin-5-one (5 mg, 0.01 mmol) in methanol (0.2 mL) was added prop-2-enenitrile (3 mg, 0.05 mmol). The reaction mixture was stirred at 50° C. for 18 hours before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₁H₃₆ClN₁₀O (M+H)⁺ m/z=599.3; found 599.3.

### Example 205: 9-(1-((6-Chloro-2-(1-methyl-1H-1,2,4-triazol-3-yl)pyridin-3-yl)amino)ethyl)-4-ethyl-7-methyl-2-(1-methylazetidin-3-yl)-2,4-dihydro-5H-pyrazolo[3,4-c]isoquinolin-5-one

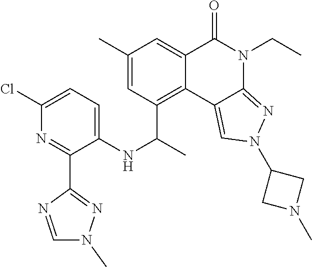 [SMILES: CCn1c(=O)c2cc(C)cc(C(C)Nc3ccc(Cl)nc3-c3ncn(C)n3)c2c2cn(C3CN(C)C3)nc21]

The titled compound was prepared using similar procedures as described for Example 201 with 1-boc-3-iodoazetidine replacing 1-boc-4-bromopiperidine in Step 1. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₁ClN₉O (M+H)⁺ m/z=532.2; found 532.2.

### Example 206: 9-(1-((6-Chloro-2-(1-methyl-1H-pyrazol-4-yl)pyridin-3-yl)amino)ethyl)-4,7-dimethyl-2-(1-methylpiperidin-4-yl)-2,4-dihydro-5H-pyrazolo[3,4-c]isoquinolin-5-one

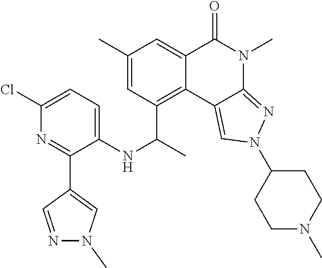 [SMILES: Cc1cc(C(C)Nc2ccc(Cl)nc2-c2cnn(C)c2)c2c(c1)c(=O)n(C)c1nn(C3CCN(C)CC3)cc21]

### Step 1. tert-butyl 4-(9-(1-((6-chloro-2-(1-methyl-1H-pyrazol-4-yl)pyridin-3-yl)amino)ethyl)-4,7-dimethyl-5-oxo-4,5-dihydro-2H-pyrazolo[3,4-c]isoquinolin-2-yl)piperidine-1-carboxylate

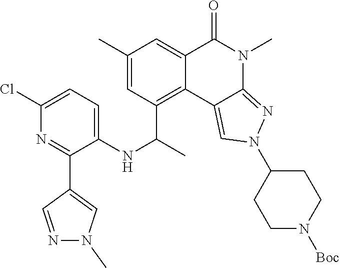 [SMILES: Cc1cc(C(C)Nc2ccc(Cl)nc2-c2cnn(C)c2)c2c(c1)c(=O)n(C)c1nn(C3CCN(C(=O)OC(C)(C)C)CC3)cc21]

The titled compound was prepared using similar procedures as described for Example 26 with 6-chloro-2-(1-methyl-1H-pyrazol-4-yl)pyridin-3-amine replacing methyl 2-aminobenzoate in Step 7 and tert-butyl 4-iodopiperidine-1-carboxylate replacing 1-iodopropane in Step 9. The mixture was diluted with water and extracted with EtOAc. The combined organics were dried with anhydrous sodium sulfate, filtered, and concentrated under reduced pressure. The residue was purified by silica gel column chromatography, eluted with hexane and ethyl acetate to provide the desired product as a white solid. LCMS calculated for C₃₃H₄₀ClN₈O₃ (M+H)⁺ m/z=631.3; found 631.3.

### Step 2. 9-(1-((6-chloro-2-(1-methyl-1H-pyrazol-4-yl)pyridin-3-yl)amino)ethyl)-4,7-dimethyl-2-(piperidin-4-yl)-2,4-dihydro-5H-pyrazolo[3,4-c]isoquinolin-5-one

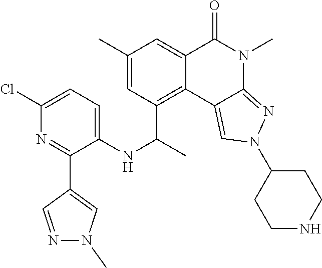 [SMILES: Cc1cc(C(C)Nc2ccc(Cl)nc2-c2cnn(C)c2)c2c(c1)c(=O)n(C)c1nn(C3CCNCC3)cc21]

tert-Butyl 4-(9-(1-((6-chloro-2-(1-methyl-1H-pyrazol-4-yl)pyridin-3-yl)amino)ethyl)-4,7-dimethyl-5-oxo-4,5-dihydro-2H-pyrazolo[3,4-c]isoquinolin-2-yl)piperidine-1-carboxylate (10 mg, 0.016 mmol) was dissolved in 1 mL of 30% TFA in DCM. After stirring for 30 min at r.t., the solvent was removed under vacuum. The residue was used directly in the next step without further purification. LCMS calculated for C₂₈H₃₂ClN₈O (M+H)⁺ m/z=531.2; found 531.2.

### Step 3. 9-(1-((6-Chloro-2-(1-methyl-1H-pyrazol-4-yl)pyridin-3-yl)amino)ethyl)-4,7-dimethyl-2-(1-methylpiperidin-4-yl)-2,4-dihydro-5H-pyrazolo[3,4-c]isoquinolin-5-one

To a solution of 9-(1-((6-chloro-2-(1-methyl-1H-pyrazol-4-yl)pyridin-3-yl)amino)ethyl)-4,7-dimethyl-2-(piperidin-4-yl)-2,4-dihydro-5H-pyrazolo[3,4-c]isoquinolin-5-one (5 mg, 0.01 mmol) in 0.5 mL of ACN and 0.1 mL AcOH was added formaldehyde (6 mg, 0.2 mmol) and sodium triacetoxyhydroborate (7.6 mg, 0.04 mml) at 0° C. After stirring at r.t. for 30 min, the mixture was diluted with water and extracted with EtOAc. The combined organics were dried with anhydrous sodium sulfate, filtered, and concentrated under reduced pressure. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₄ClN₈O (M+H)⁺ m/z=545.3; found 545.3.

### Examples 207-208

Examples 207-208 in Table 30 were prepared similarly as described for Example 206

TABLE 30

| Ex- | | | LCMS |
|---|---|---|---|
| ample | | | m/z |
| # | Chemical name | Structure | (M + H)⁺ |
| 207 | 9-(1-((6-chloro-2-(1-methyl-1H- pyrazol-4-yl)pyridin-3- yl)amino)ethyl)-2-(1- isopropylpiperidin-4-yl)-4,7- dimethyl-2,4-dihydro-5H- pyrazolo[3,4-c]isoquinolin-5-one | 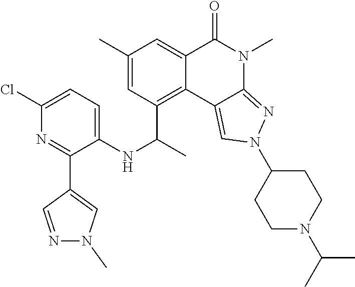 [SMILES: Cc1cc(C(C)Nc2ccc(Cl)nc2-c2cnn(C)c2)c2c(c1)c(=O)n(C)c1nn(C3CCN(C(C)C)CC3)cc21] | 573.3 |
| 208 | 9-(1-((6-chloro-2-(1-methyl-1H- 1,2,4-triazol-3-yl)pyridin-3- yl)amino)ethyl)-4,7-dimethyl-2-(1- methylpiperidin-4-yl)-2,4-dihydro- 5H-pyrazolo[3,4-c]isoquinolin-5- one | 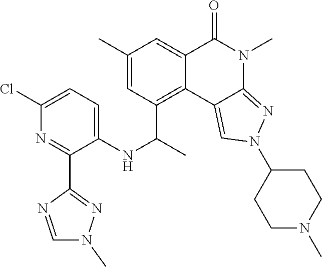 [SMILES: Cc1cc(C(C)Nc2ccc(Cl)nc2-c2ncn(C)n2)c2c(c1)c(=O)n(C)c1nn(C3CCN(C)CC3)cc21] | 546.2 |

### Example 209: 2-(1-Acetylpiperidin-4-yl)-9-(1-((6-chloro-2-(1-methyl-1H-pyrazol-4-yl)pyridin-3-yl)amino)ethyl)-4,7-dimethyl-2,4-dihydro-5H-pyrazolo[3,4-c]isoquinolin-5-one

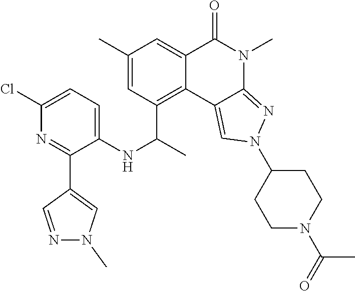 [SMILES: CC(=O)N1CCC(n2cc3c4c(C(C)Nc5ccc(Cl)nc5-c5cnn(C)c5)cc(C)cc4c(=O)n(C)c3n2)CC1]

The titled compound was prepared using similar procedures as described for Example 202 with 9-(1-((6-chloro-2-(1-methyl-1H-pyrazol-4-yl)pyridin-3-yl)amino)ethyl)-4,7-dimethyl-2-(piperidin-4-yl)-2,4-dihydro-5H-pyrazolo[3,4-c]isoquinolin-5-one (Example 206, step 2) replacing 9-(1-((6-chloro-2-(1-methyl-1H-1,2,4-triazol-3-yl)pyridin-3-yl)amino)ethyl)-4-ethyl-7-methyl-2-(piperidin-4-yl)-2,4-dihydro-5H-pyrazolo[3,4-c]isoquinolin-5-one. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₀H₃₄ClN₈O₂ (M+H)⁺ m/z=573.2; found 573.2.

### Examples 210-211

Examples 210-211 in Table 31 were prepared similarly as described for Example 209.

TABLE 31

| Ex- | | | LCMS |
|---|---|---|---|
| ample | | | m/z |
| # | Chemical name | Structure | (M + H)⁺ |
| 210 | ethyl 4-(9-(1-((6-chloro-2-(1- methyl-1H-pyrazol-4- yl)pyridin-3-yl)amino)ethyl)- 4,7-dimethyl-5-oxo-4,5- dihydro-2H-pyrazolo[3,4- c]isoquinolin-2-yl)piperidine- 1-carboxylate | 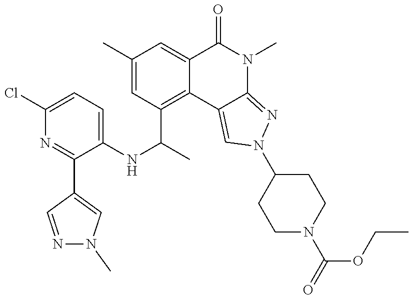 [SMILES: CCOC(=O)N1CCC(n2cc3c4c(C(C)Nc5ccc(Cl)nc5-c5cnn(C)c5)cc(C)cc4c(=O)n(C)c3n2)CC1] | 603.3 |
| 211 | 4-(9-(1-((6-chloro-2-(1- methyl-1H-pyrazol-4- yl)pyridin-3-yl)amino)ethyl)- 4,7-dimethyl-5-oxo-4,5- dihydro-2H-pyrazolo[3,4- c]isoquinolin-2-yl)-N,N- dimethylpiperidine-1- carboxamide | 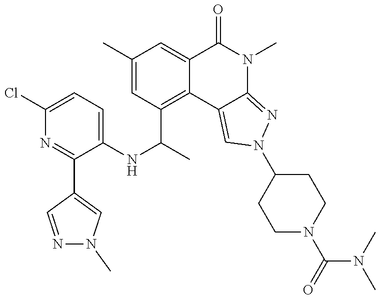 [SMILES: Cc1cc(C(C)Nc2ccc(Cl)nc2-c2cnn(C)c2)c2c(c1)c(=O)n(C)c1nn(C3CCN(C(=O)N(C)C)CC3)cc21] | 602.3 |

### Example 212: 9-(1-((6-Chloro-2-(1-methyl-1H-pyrazol-4-yl)pyridin-3-yl)amino)ethyl)-2-(1-(2,2-difluoroethyl)piperidin-4-yl)-4,7-dimethyl-2,4-dihydro-5H-pyrazolo[3,4-c]isoquinolin-5-one

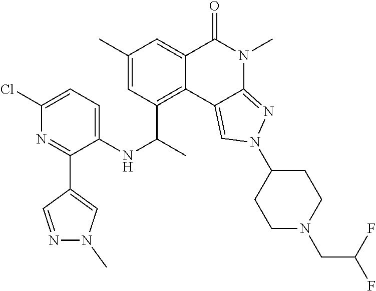 [SMILES: Cc1cc(C(C)Nc2ccc(Cl)nc2-c2cnn(C)c2)c2c(c1)c(=O)n(C)c1nn(C3CCN(CC(F)F)CC3)cc21]

The titled compound was prepared using similar procedures as described for Example 142 with 9-(1-((6-chloro-2-(1-methyl-1H-pyrazol-4-yl)pyridin-3-yl)amino)ethyl)-4,7-dimethyl-2-(piperidin-4-yl)-2,4-dihydro-5H-pyrazolo[3,4-c]isoquinolin-5-one (Example 206, step 2) replacing 9-(1-((6-chloro-2-(1-methyl-1H-pyrazol-4-yl)pyridin-3-yl)amino)ethyl)-4,7-dimethyl-3-(pyrrolidin-3-yl)-3,4-dihydro-5H-pyrazolo[3,4-c]isoquinolin-5-one. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₀H₃₄ClF₂N₈O (M+H)⁺ m/z=595.2; found 595.2.

### Example 213: 3-[6-Chloro-3-[1-[7-methyl-5-oxo-3-[(3S)-tetrahydrofuran-3-yl]-4-(trideuteriomethyl)pyrazolo[3,4-c]isoquinolin-9-yl]ethylamino]-2-pyridyl]-4H-1,2,4-oxadiazol-5-one

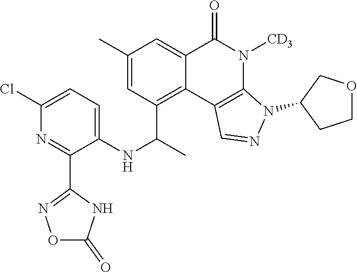 [SMILES: [2H]C([2H])([2H])n1c(=O)c2cc(C)cc(C(C)Nc3ccc(Cl)nc3-c3noc(=O)[nH]3)c2c2cnn([C@H]3CCOC3)c21]

### Step 1. 9-(1-bromoethyl)-7-methyl-4-(methyl-d3)-3-((S)-tetrahydrofuran-3-yl)-3,4-dihydro-5H-pyrazolo[3,4-c]isoquinolin-5-one

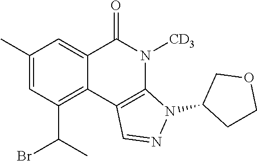 [SMILES: [2H]C([2H])([2H])n1c(=O)c2cc(C)cc(C(C)Br)c2c2cnn([C@H]3CCOC3)c21]

The title compound was prepared using similar procedures as described for Example 81 with with [(3R)-tetrahydrofuran-3-yl]hydrazine replacing tert-butyl 3-hydrazineylazetidine-1-carboxylate in Step 2, and CD₃I replacing iodomethane in step 5.

### Step 2. 3-[6-chloro-3-[1-[7-methyl-5-oxo-3-[(3S)-tetrahydrofuran-3-yl]-4-(trideuteriomethyl)pyrazolo[3,4-c]isoquinolin-9-yl]ethylamino]-2-pyridyl]-4H-1,2,4-oxadiazol-5-one

9-(1-Bromoethyl)-7-methyl-4-(methyl-d3)-3-((S)-tetrahydrofuran-3-yl)-3,4-dihydro-5H-pyrazolo[3,4-c]isoquinolin-5-one (20 mg, 0.06 mmol) in 2 dram vial was added DMF (0.3 mL), and 3-(3-amino-6-chloropyridin-2-yl)-1,2,4-oxadiazol-5(4H)-one (20 mg, 0.09 mmol). The mixture was heated to 80° C. for 2 h. Upon cooling to room temperature, the mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₂₂D₃ClN₇O₄ (M+H)⁺ m/z=525.2; found 525.2.

### Examples 214-220

Examples 214-220 in Table 32 were prepared similarly as described for Example 213.

TABLE 32

| Ex- | | | LCMS |
|---|---|---|---|
| ample | | | m/z |
| # | Chemical name | Structure | (M + H)⁺ |
| 214 | 9-(1-((6-chloro-2-(1-methyl-1H- pyrazol-4-yl)pyridin-3- yl)amino)ethyl)-7-methyl-4-(methyl- d3)-3-((S)-tetrahydrofuran-3-yl)-3,4- dihydro-5H-pyrazolo[3,4- c]isoquinolin-5-one | 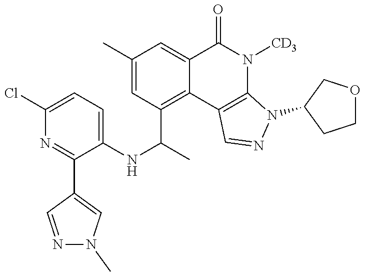 [SMILES: [2H]C([2H])([2H])n1c(=O)c2cc(C)cc(C(C)Nc3ccc(Cl)nc3-c3cnn(C)c3)c2c2cnn([C@H]3CCOC3)c21] | 521.2 |
| 215 | 3-(6-chloro-3-((1-(4-ethyl-7-methyl- 5-oxo-3-((S)-tetrahydrofuran-3-yl)- 4,5-dihydro-3H-pyrazolo[3,4- c]isoquinolin-9- yl)ethyl)amino)pyridin-2-yl)-1,2,4- oxadiazol-5(4H)-one | 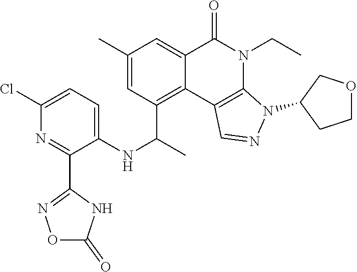 [SMILES: CCn1c(=O)c2cc(C)cc(C(C)Nc3ccc(Cl)nc3-c3noc(=O)[nH]3)c2c2cnn([C@H]3CCOC3)c21] | 536.2 |
| 216 | 9-(1-((6-chloro-2-(1-methyl-1H- 1,2,4-triazol-3-yl)pyridin-3- yl)amino)ethyl)-4-ethyl-7-methyl-3- ((S)-tetrahydrofuran-3-yl)-3,4- dihydro-5H-pyrazolo[3,4- c]isoquinolin-5-one | 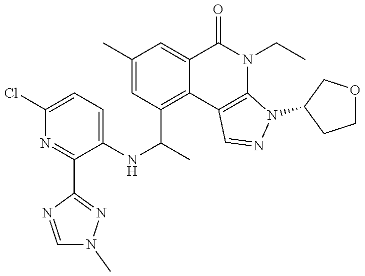 [SMILES: CCn1c(=O)c2cc(C)cc(C(C)Nc3ccc(Cl)nc3-c3ncn(C)n3)c2c2cnn([C@H]3CCOC3)c21] | 533.2 |
| 217 | 9-(1-((6-chloro-2-(1-(1- methylpiperidin-4-yl)-1H-pyrazol-4- yl)pyridin-3-yl)amino)ethyl)-4-ethyl- 7-methyl-3-((S)-tetrahydrofuran-3- yl)-3,4-dihydro-5H-pyrazolo[3,4- c]isoquinolin-5-one | 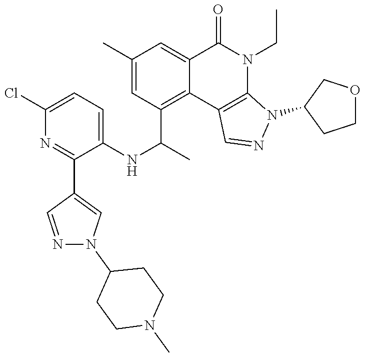 [SMILES: CCn1c(=O)c2cc(C)cc(C(C)Nc3ccc(Cl)nc3-c3cnn(C4CCN(C)CC4)c3)c2c2cnn([C@H]3CCOC3)c21] | 615.3 |
| 218 | 9-(1-((6-chloro-2′-methyl-[2,4′- bipyridin]-3-yl)amino)ethyl)-4-ethyl- 7-methyl-3-((S)-tetrahydrofuran-3- yl)-3,4-dihydro-5H-pyrazolo[3,4- c]isoquinolin-5-one | 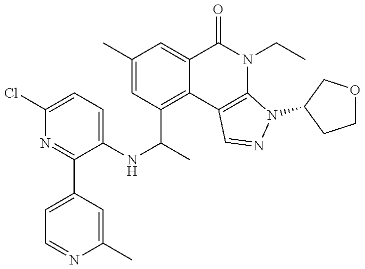 [SMILES: CCn1c(=O)c2cc(C)cc(C(C)Nc3ccc(Cl)nc3-c3ccnc(C)c3)c2c2cnn([C@H]3CCOC3)c21] | 543.2 |
| 219 | 9-(1-((6-chloro-1′-methyl-6′-oxo- 1′,6′-dihydro-[2,3′-bipyridin]-3- yl)amino)ethyl)-4-ethyl-7-methyl-3- ((S)-tetrahydrofuran-3-yl)-3,4- dihydro-5H-pyrazolo[3,4- c]isoquinolin-5-one | 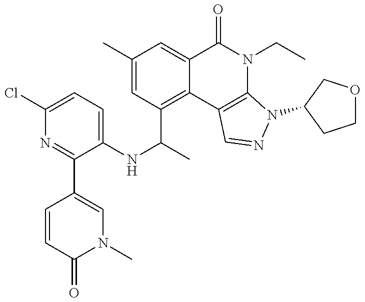 [SMILES: CCn1c(=O)c2cc(C)cc(C(C)Nc3ccc(Cl)nc3-c3ccc(=O)n(C)c3)c2c2cnn([C@H]3CCOC3)c21] | 559.2 |
| 220 | 6-chloro-3-((1-(4-ethyl-7-methyl-5- oxo-3-((S)-tetrahydrofuran-3-yl)-4,5- dihydro-3H-pyrazolo[3,4- c]isoquinolin-9-yl)ethyl)amino)-N- methyl-[2,3′-bipyridine]-6′- carboxamide | 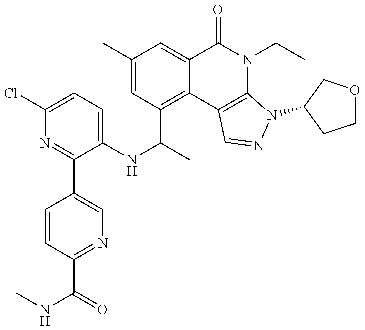 [SMILES: CCn1c(=O)c2cc(C)cc(C(C)Nc3ccc(Cl)nc3-c3ccc(C(=O)NC)nc3)c2c2cnn([C@H]3CCOC3)c21] | 586.2 |

### Example 221: 9-(1-((6-Chloro-2-(1-methyl-1H-1,2,4-triazol-3-yl)pyridin-3-yl)amino)ethyl)-4-cyclopropyl-3-ethyl-7-methyl-3,4-dihydro-5H-pyrazolo[3,4-c]isoquinolin-5-one

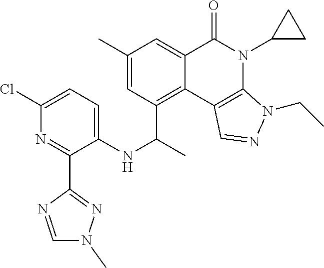 [SMILES: CCn1ncc2c3c(C(C)Nc4ccc(Cl)nc4-c4ncn(C)n4)cc(C)cc3c(=O)n(C3CC3)c21]

The titled compound was prepared using similar procedures as described for Example 16 with 9-bromo-4-cyclopropyl-3-ethyl-7-methyl-pyrazolo[3,4-c]isoquinolin-5-one (intermediate 52) replacing 9-bromo-3,4,7-trimethyl-3,4-dihydro-5H-pyrazolo[3,4-c]isoquinolin-5-one in Step 4, and 6-chloro-2-(1-methyl-1H-1,2,4-triazol-3-yl)pyridin-3-amine replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₂₈ClN₈O (M+H)⁺ m/z=503.2; found 503.2.

### Example 222

Example 222 in Table 33 were prepared similarly as described for Example 221

TABLE 33

| Ex- | | | LCMS |
|---|---|---|---|
| ample | | | m/z |
| # | Chemical name | Structure | (M + H)⁺ |
| 222 | 9-[1-[[6-chloro-2-(1-methyl-6-oxo-3- pyridyl)-3-pyridyl]amino]ethyl]-4- cyclopropyl-3-ethyl-7-methyl- pyrazolo[3,4-c]isoquinolin-5-one | 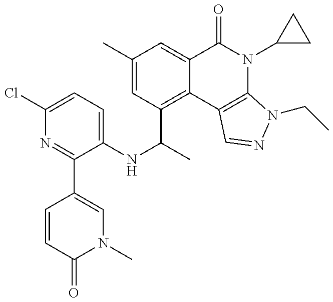 [SMILES: CCn1ncc2c3c(C(C)Nc4ccc(Cl)nc4-c4ccc(=O)n(C)c4)cc(C)cc3c(=O)n(C3CC3)c21] | 529.2 |

### Example 223: 9-[1-[[6-Chloro-2-(1-methyl-1,2,4-triazol-3-yl)-3-pyridyl]amino]ethyl]-3-ethyl-7-methyl-4-tetrahydrofuran-3-yl-pyrazolo[3,4-c]isoquinolin-5-one

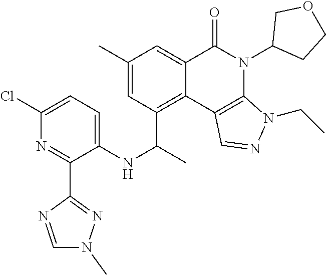 [SMILES: CCn1ncc2c3c(C(C)Nc4ccc(Cl)nc4-c4ncn(C)n4)cc(C)cc3c(=O)n(C3CCOC3)c21]

The titled compound was prepared using similar procedures as described for Example 16 with 9-bromo-3-ethyl-7-methyl-4-(tetrahydrofuran-3-yl)-3,4-dihydro-5H-pyrazolo[3,4-c]isoquinolin-5-one (intermediate 53) replacing 9-bromo-3,4,7-trimethyl-3,4-dihydro-5H-pyrazolo[3,4-c]isoquinolin-5-one in Step 4, and 6-chloro-2-(1-methyl-1H-1,2,4-triazol-3-yl)pyridin-3-amine replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₀ClN₈O₂ (M+H)⁺ m/z=533.2; found 533.2.

### Example 224: 9-[1-[[6-Chloro-2-(1-methyl-1,2,4-triazol-3-yl)-3-pyridyl]amino]ethyl]-3-ethyl-4-(2-hydroxyethyl)-7-methyl-pyrazolo[3,4-c]isoquinolin-5-one

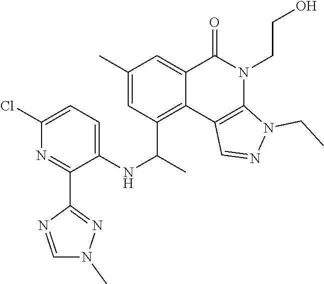 [SMILES: CCn1ncc2c3c(C(C)Nc4ccc(Cl)nc4-c4ncn(C)n4)cc(C)cc3c(=O)n(CCO)c21]

The titled compound was prepared using similar procedures as described for Example 16 with 1-ethyl-1H-pyrazol-5-amine replacing 1-methyl-1H-pyrazol-5-amine in Step 1 and tert-Butyl(2-iodoethoxy)dimethylsilane replacing methyl iodide in step 2, 6-chloro-2-(1-methyl-1,2,4-triazol-3-yl)pyridin-3-amine replacing 2-aminobenzoic acid in Step 7. The crude product from Step 7 was dissolved in 4N HCl and stirred for 30 min before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₂₈ClN₈O₂ (M+H)⁺ m/z=507.2; found 507.1.

### Example 225: 4-(6-Chloro-3-(((3-ethyl-4,7-dimethyl-5-oxo-4,5-dihydro-3H-pyrazolo[3,4-c]isoquinolin-9-yl)methyl)amino)pyridin-2-yl)-2-fluoro-N-methylbenzamide

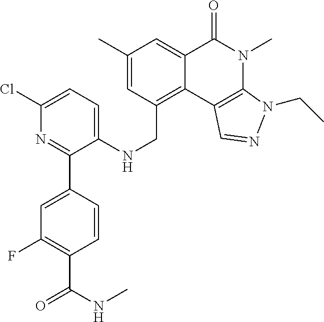 [SMILES: CCn1ncc2c3c(CNc4ccc(Cl)nc4-c4ccc(C(=O)NC)c(F)c4)cc(C)cc3c(=O)n(C)c21]

### Step 1: 3-ethyl-4,7-dimethyl-5-oxo-4,5-dihydro-3H-pyrazolo[3,4-c]isoquinoline-9-carbaldehyde

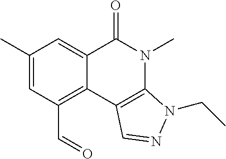 [SMILES: CCn1ncc2c3c(C=O)cc(C)cc3c(=O)n(C)c21]

To a stirred solution of 3-ethyl-4,7-dimethyl-9-vinyl-3,4-dihydro-5H-pyrazolo[3,4-c]isoquinolin-5-one (260 mg, 0.97 mmol) (see example 76, step 2), sodium periodate (1.04 g, 4.86 mmol) and 2,6-dimethylpyridine (0.23 mL, 1.95 mmol) in 10 mL THF and 2 mL H₂O was added OsO₄ (19.8 mg, 0.08 mmol) (4% aq. solution) dropwise and the resulting reaction mixture was kept stirred at rt for 3 h. LCMS analysis indicated the reaction was complete. The reaction mixture was diluted with water and extracted with EtOAc. The combined organics were washed with sat. NaCl and dried with anhydrous sodium sulfate, filtered, and concentrated under reduced pressure to afford the desired product (0.16 g, 58%) which was directly used in the following step without further purification. LCMS calculated for C₁₅H₁₆N₃O₂ (M+H)⁺ m/z=270.1; found 270.1.

### Step 2: 3-ethyl-9-(hydroxymethyl)-4,7-dimethyl-3,4-dihydro-5H-pyrazolo[3,4-c]isoquinolin-5-one

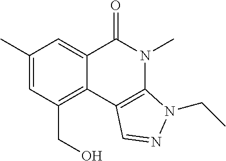 [SMILES: CCn1ncc2c3c(CO)cc(C)cc3c(=O)n(C)c21]

To a mixture of 3-ethyl-4,7-dimethyl-5-oxo-4,5-dihydro-3H-pyrazolo[3,4-c]isoquinoline-9-carbaldehyde (150 mg, 0.56 mmol) in THF (6 mL) was added LAH (1.1 mL, 1.1 mmol, 1 M solution in THF) at −78° C. The resulting mixture was stirred at same temperature for 10 min, and then warmed to −40° C. before quenched with sat. NH₄Cl. The mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a white solid (125 mg, 83%). LCMS calculated for C₁₅H₁₈N₃O₂ (M+H)⁺ m/z=272.1; found 272.1.

### Step 3: 9-(bromomethyl)-3-ethyl-4,7-dimethyl-3,4-dihydro-5H-pyrazolo[3,4-c]isoquinolin-5-one

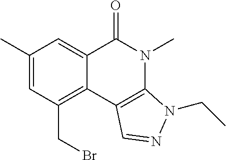 [SMILES: CCn1ncc2c3c(CBr)cc(C)cc3c(=O)n(C)c21]

To a mixture of 3-ethyl-9-(hydroxymethyl)-4,7-dimethyl-3,4-dihydro-5H-pyrazolo[3,4-c]isoquinolin-5-one (80 mg, 0.29 mmol) in DCM (1 mL) was added PBr₃ (160 mg, 0.59 mmol). The resulting mixture was stirred at rt for 2 h. The mixture was then quenched with sat. NaHCO₃ and extracted with DCM. The combined organics were washed with sat. NaCl, dried with anhydrous sodium sulfate, filtered, and concentrated under reduced pressure. The residue was used directly in the next step without further purification.

### Step 4: 9-(((2-bromo-6-chloropyridin-3-yl)amino)methyl)-3-ethyl-4,7-dimethyl-3,4-dihydro-5H-pyrazolo[3,4-c]isoquinolin-5-one

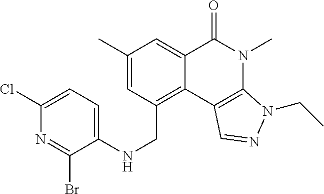 [SMILES: CCn1ncc2c3c(CNc4ccc(Cl)nc4Br)cc(C)cc3c(=O)n(C)c21]

To the solution of 9-(bromomethyl)-3-ethyl-4,7-dimethyl-3,4-dihydro-5H-pyrazolo[3,4-c]isoquinolin-5-one (97 mg, 0.29 mmol) in DMF (0.3 mL) was added 2-bromo-6-chloro-pyridin-3-amine (184 mg, 0.88 mmol). The mixture was heated to 80° C. for 2 h. Upon cooling to room temperature, the mixture was diluted with water and extracted with ethyl acetate (3×). The combined organics were washed with water, sat. NaCl, and dried with anhydrous sodium sulfate, filtered, and concentrated under reduced pressure. The residue was purified by silica gel column chromatography, eluted with DCM and MeOH to provide the desired product as a light yellow solid (75 mg, 54%). LCMS calculated for C₂₀H₂₀BrClN₅O (M+H)⁺ m/z=460.0; found 460.0.

### Step 5: 4-(6-chloro-3-(((3-ethyl-4,7-dimethyl-5-oxo-4,5-dihydro-3H-pyrazolo[3,4-c]isoquinolin-9-yl)methyl)amino)pyridin-2-yl)-2-fluoro-N-methylbenzamide

A mixture of 9-(((2-bromo-6-chloropyridin-3-yl)amino)methyl)-3-ethyl-4,7-dimethyl-3,4-dihydro-5H-pyrazolo[3,4-c]isoquinolin-5-one (10.0 mg, 0.02 mmol), [3-fluoro-4-(methylcarbamoyl)phenyl]boronic acid (4.27 mg, 0.02 mmol), cesium carbonate (21.2 mg, 0.07 mmol) and tetrakis(triphenylphosphine)palladium(0) (5.0 mg, 0.004 mmol) in a 5 to 1 mixture of toluene and water (0.2 mL) was heated at 100° C. for 1 h under nitrogen atmosphere. Upon cooling to room temperature, the mixture was concentrated under reduced pressure. The residue was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₂₇ClFN₆O₂ (M+H)⁺ m/z=533.2; found: 533.2;

### Example 226: 9-(1-((6-Chloro-2-(3-methyl-1H-pyrazol-4-yl)pyridin-3-yl)oxy)ethyl)-3-ethyl-4,7-dimethyl-3,4-dihydro-5H-pyrazolo[3,4-c]isoquinolin-5-one

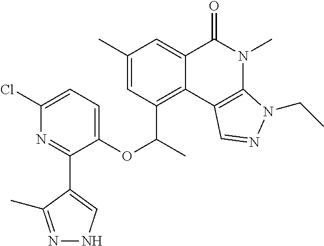 [SMILES: CCn1ncc2c3c(C(C)Oc4ccc(Cl)nc4-c4c[nH]nc4C)cc(C)cc3c(=O)n(C)c21]

### Step 1: 9-(1-bromoethyl)-3-ethyl-4,7-dimethyl-3,4-dihydro-5H-pyrazolo[3,4-c]isoquinolin-5-one

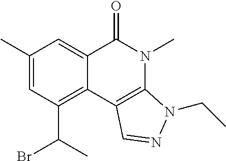 [SMILES: CCn1ncc2c3c(C(C)Br)cc(C)cc3c(=O)n(C)c21]

The title compound was prepared using similar procedures as described for Example 16 with 1-ethyl-1H-pyrazol-5-amine replacing 1-methyl-1H-pyrazol-5-amine in step 1. The crude product was used in the next step without purification.

### Step 2: 9-(1-((2-bromo-6-chloropyridin-3-yl)oxy)ethyl)-3-ethyl-4,7-dimethyl-3,4-dihydro-5H-pyrazolo[3,4-c]isoquinolin-5-one

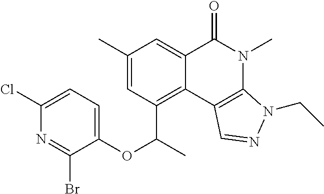 [SMILES: CCn1ncc2c3c(C(C)Oc4ccc(Cl)nc4Br)cc(C)cc3c(=O)n(C)c21]

To a solution of 9-(1-bromoethyl)-3-ethyl-4,7-dimethyl-3,4-dihydro-5H-pyrazolo[3,4-c]isoquinolin-5-one (115 mg, 0.33 mmol) in DMF (0.5 mL) was added 2-bromo-6-chloro-pyridin-3-ol (208.2 mg, 1.0 mmol) and potassium carbonate (138.0 mg, 1.0 mmol). The mixture was heated to 60° C. for 16 h. Upon cooling to room temperature, the mixture was diluted with water and extracted with ethyl acetate (3×). The combined organics were washed with water, sat. NaCl, and dried with anhydrous sodium sulfate, filtered, and concentrated under reduced pressure. The residue was purified by silica gel column chromatography, eluted with DCM and MeOH to provide the desired product as a light yellow solid (80 mg, 50%). LCMS calculated for C₂₁H₂₁BrClN₄O₂ (M+H)⁺ m/z=475.1; found 475.1.

### Step 3: 9-(1-((6-chloro-2-(3-methyl-1H-pyrazol-4-yl)pyridin-3-yl)oxy)ethyl)-3-ethyl-4,7-dimethyl-3,4-dihydro-5H-pyrazolo[3,4-c]isoquinolin-5-one

A mixture of 9-(1-((2-bromo-6-chloropyridin-3-yl)oxy)ethyl)-3-ethyl-4,7-dimethyl-3,4-dihydro-5H-pyrazolo[3,4-c]isoquinolin-5-one (10 mg, 0.02 mmol), 3-methyl-4-(4,4,5,5-tetramethyl-1,3,2-dioxaborolan-2-yl)-1H-pyrazole (4.4 mg, 0.02 mmol), cesium carbonate (20.5 mg, 0.06 mmol) and tetrakis(triphenylphosphine)palladium(0) (4.9 mg, 0.004 mmol) in a 5 to 1 mixture of dioxane and water (0.2 mL) was heated at 100° C. for 1 h under nitrogen atmosphere. Upon cooling to room temperature, the mixture was concentrated under reduced pressure. The residue was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₂₆ClN₆O₂ (M+H)⁺ m/z=477.2; found: 477.2;

### Example 227: 3-(6-Chloro-3-((1-(3-ethyl-7-methyl-4-(methyl-d3)-5-oxo-4,5-dihydro-3H-pyrazolo[3,4-c]isoquinolin-9-yl)ethyl)amino)pyridin-2-yl)-1,2,4-oxadiazol-5(4H)-one

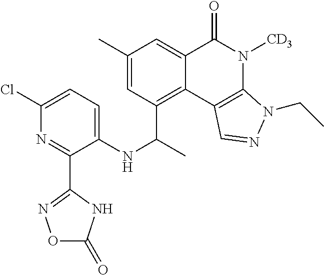 [SMILES: [2H]C([2H])([2H])n1c(=O)c2cc(C)cc(C(C)Nc3ccc(Cl)nc3-c3noc(=O)[nH]3)c2c2cnn(CC)c21]

The titled compound was prepared using similar procedures as described for Example 16 with iodomethane-d3 replacing iodomethane in Step 2 and 3-(3-amino-6-chloropyridin-2-yl)-1,2,4-oxadiazol-5(4H)-one replacing 2-aminobenzoic acid in Step 7. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₂₀D₃ClN₇O₃ (M+H)⁺ m/z=483.2; found 483.2.

### Examples 228-235

Examples 228-235 in Table 34 were prepared similarly as described for Example 18

TABLE 34

| | | | LCMS |
|---|---|---|---|
| Example | | | m/z |
| # | Chemical name | Structure | (M + H)⁺ |
| 228 | 9-(1-((6-chloro-2-(1-methyl-1H- 1,2,4-triazol-3-yl)pyridin-3- yl)amino)ethyl)-3-ethyl-4,7- dimethyl-3,4-dihydro-5H- pyrazolo[3,4-c]isoquinolin-5-one | 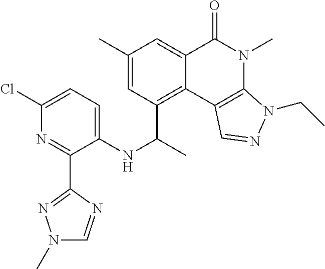 [SMILES: CCn1ncc2c3c(C(C)Nc4ccc(Cl)nc4-c4ncn(C)n4)cc(C)cc3c(=O)n(C)c21] | 477.2 |
| 229 | 9-(1-((6-chloro-2-(4-methyl-1H- 1,2,4-triazol-3-yl)pyridin-3- yl)amino)ethyl)-3-ethyl-4,7- dimethyl-3,4-dihydro-5H- pyrazolo[3,4-c]isoquinolin-5-one | 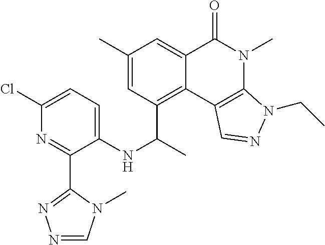 [SMILES: CCn1ncc2c3c(C(C)Nc4ccc(Cl)nc4-c4nncn4C)cc(C)cc3c(=O)n(C)c21] | 477.2 |
| 230 | 9-(1-((6-chloro-2-(1H-tetrazol-5- yl)pyridin-3-yl)amino)ethyl)-3- ethyl-4,7-dimethyl-3,4-dihydro- 5H-pyrazolo[3,4-c]isoquinolin-5- one | 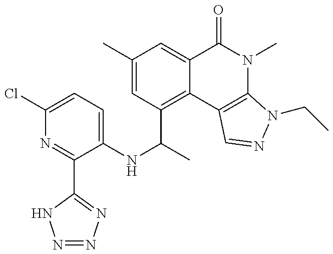 [SMILES: CCn1ncc2c3c(C(C)Nc4ccc(Cl)nc4-c4nnn[nH]4)cc(C)cc3c(=O)n(C)c21] | 464.2 |
| 231 | 9-(1-((6-chloro-2-(1-methyl-1H- 1,2,3-triazol-5-yl)pyridin-3- yl)amino)ethyl)-3-ethyl-4,7- dimethyl-3,4-dihydro-5H- pyrazolo[3,4-c]isoquinolin-5-one | 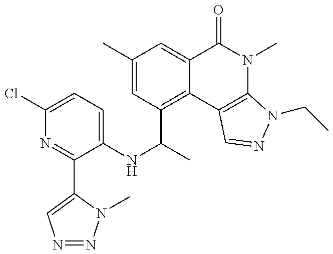 [SMILES: CCn1ncc2c3c(C(C)Nc4ccc(Cl)nc4-c4cnnn4C)cc(C)cc3c(=O)n(C)c21] | 477.2 |
| 232 | 4-(6-chloro-3-((1-(3-ethyl-4,7- dimethyl-5-oxo-4,5-dihydro-3H- pyrazolo[3,4-c]isoquinolin-9- yl)ethyl)amino)pyridin-2- yl)benzoic acid | 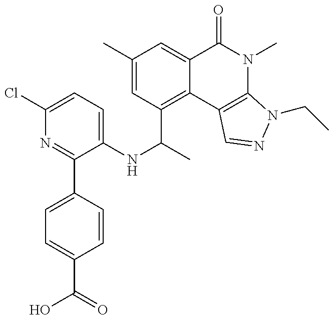 [SMILES: CCn1ncc2c3c(C(C)Nc4ccc(Cl)nc4-c4ccc(C(=O)O)cc4)cc(C)cc3c(=O)n(C)c21] | 516.2 |
| 233 | 4-(4-(6-chloro-3-((1-(3-ethyl-4,7- dimethyl-5-oxo-4,5-dihydro-3H- pyrazolo[3,4-c]isoquinolin-9- yl)ethyl)amino)pyridin-2-yl)-1H- pyrazol-1-yl)-N,N- dimethylpiperidine-1-carboxamide | 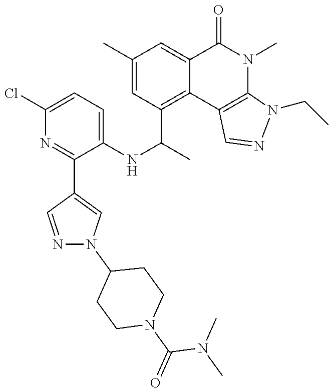 [SMILES: CCn1ncc2c3c(C(C)Nc4ccc(Cl)nc4-c4cnn(C5CCN(C(=O)N(C)C)CC5)c4)cc(C)cc3c(=O)n(C)c21] | 616.3 |
| 234 | 3-(4-(4-(6-chloro-3-((1-(3-ethyl- 4,7-dimethyl-5-oxo-4,5-dihydro- 3H-pyrazolo[3,4-c]isoquinolin-9- yl)ethyl)amino)pyridin-2-yl)-1H- pyrazol-1-yl)piperidin-1- yl)propanenitrile | 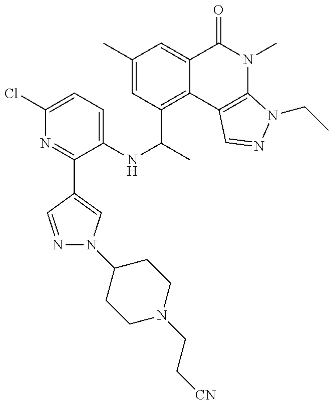 [SMILES: CCn1ncc2c3c(C(C)Nc4ccc(Cl)nc4-c4cnn(C5CCN(CCC#N)CC5)c4)cc(C)cc3c(=O)n(C)c21] | 598.3 |
| 235 | 3-ethyl-4,7-dimethyl-9-(1-((6- methyl-2-(3-methyl-1H-pyrazol-4- yl)pyridin-3-yl)amino)ethyl)-3,4- dihydro-5H-pyrazolo[3,4- c]isoquinolin-5-one | 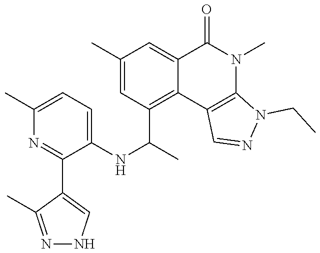 [SMILES: CCn1ncc2c3c(C(C)Nc4ccc(C)nc4-c4c[nH]nc4C)cc(C)cc3c(=O)n(C)c21] | 456.3 |

### Examples 236-240

Examples 236-240 in Table 35 were prepared similarly as described for Example 60.

TABLE 35

| | | | LCMS |
|---|---|---|---|
| Example | | | m/z |
| # | Chemical name | Structure | (M + H)⁺ |
| 236 | 9-(1-((6-chloro-2-(1-methyl-1H- 1,2,4-triazol-3-yl)pyridin-3- yl)amino)ethyl)-4-(2,2- difluoroethyl)-3-ethyl-7-methyl- 3,4-dihydro-5H-pyrazolo[3,4- c]isoquinolin-5-one | 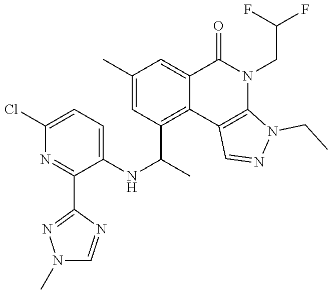 [SMILES: CCn1ncc2c3c(C(C)Nc4ccc(Cl)nc4-c4ncn(C)n4)cc(C)cc3c(=O)n(CC(F)F)c21] | 527.2 |
| 237 | 9-(1-((6-chloro-2′-methyl-[2,4′- bipyridin]-3-yl)amino)ethyl)-4- (2,2-difluoroethyl)-3-ethyl-7- methyl-3,4-dihydro-5H- pyrazolo[3,4-c]isoquinolin-5-one | 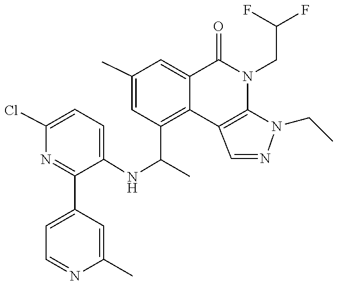 [SMILES: CCn1ncc2c3c(C(C)Nc4ccc(Cl)nc4-c4ccnc(C)c4)cc(C)cc3c(=O)n(CC(F)F)c21] | 537.2 |
| 238 | 9-(1-((6-chloro-2-(pyrimidin-5- yl)pyridin-3-yl)amino)ethyl)-4- (2,2-difluoroethyl)-3-ethyl-7- methyl-3,4-dihydro-5H- pyrazolo[3,4-c]isoquinolin-5-one | 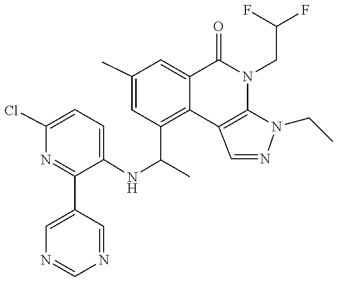 [SMILES: CCn1ncc2c3c(C(C)Nc4ccc(Cl)nc4-c4cncnc4)cc(C)cc3c(=O)n(CC(F)F)c21] | 524.2 |
| 239 | 9-(1-((6-chloro-1′-methyl-6′-oxo- 1′,6′-dihydro-[2,3′-bipyridin]-3- yl)amino)ethyl)-4-(2,2- difluoroethyl)-3-ethyl-7-methyl- 3,4-dihydro-5H-pyrazolo[3,4- c]isoquinolin-5-one | 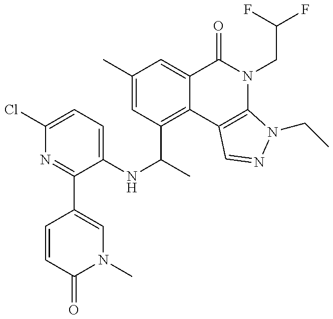 [SMILES: CCn1ncc2c3c(C(C)Nc4ccc(Cl)nc4-c4ccc(=O)n(C)c4)cc(C)cc3c(=O)n(CC(F)F)c21] | 553.2 |
| 240 | 6-chloro-3-((1-(4-(2,2- difluoroethyl)-3-ethyl-7-methyl-5- oxo-4,5-dihydro-3H-pyrazolo[3,4- c]isoquinolin-9-yl)ethyl)amino)-N- methyl-[2,3′-bipyridine]-6′- carboxamide | 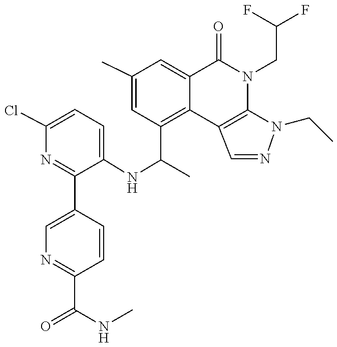 [SMILES: CCn1ncc2c3c(C(C)Nc4ccc(Cl)nc4-c4ccc(C(=O)NC)nc4)cc(C)cc3c(=O)n(CC(F)F)c21] | 580.2 |

### Example 241: 9-[1-[[2-(1-Acetyl-4-piperidyl)-6-chloro-3-pyridyl]amino]ethyl]-3-ethyl-4,7-dimethyl-pyrazolo[3,4-c]isoquinolin-5-one

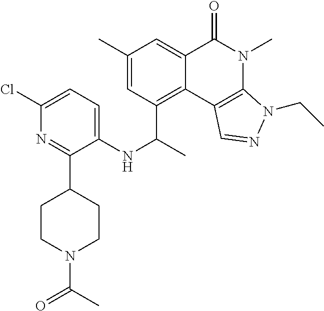 [SMILES: CCn1ncc2c3c(C(C)Nc4ccc(Cl)nc4C4CCN(C(C)=O)CC4)cc(C)cc3c(=O)n(C)c21]

### Step 1: tert-butyl 4-(3-amino-6-chloro-2-pyridyl)piperidine-1-carboxylate

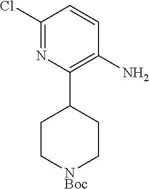 [SMILES: CC(C)(C)OC(=O)N1CCC(c2nc(Cl)ccc2N)CC1]

To a solution of 2-bromo-6-chloro-pyridin-3-amine (300 mg, 1.45 mmol) in 4 mL of THF was added Pd(dppf)Cl₂ (106 mg, 0.14 mmol), followed by (1-tert-butoxycarbonyl-4-piperidyl)-iodo-zinc (0.5 M THF solution, 3.5 mL, 1.74 mmol) dropwise at rt. The resulting solution was then heated to 70° C. for 12 h under nitrogen gas. Upon cooling down to r.t. the crude mixture was diluted with DCM and washed with water. The aqueous layer was extracted with DCM for 3 times. The combined organic layer was washed with brine and dried over sodium sulfate. Column purified: EtOAc/DCM=0% to 100% to get desired product. LCMS calculated for C₁₁H₁₅ClN₃O₂ [M−56+H]⁺256.1, found 256.1.

### Step 2: 9-[1-[[6-chloro-2-(4-piperidyl)-3-pyridyl]amino]ethyl]-3-ethyl-4,7-dimethyl-pyrazolo[3,4-c]isoquinolin-5-one

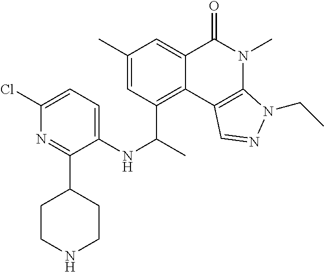 [SMILES: CCn1ncc2c3c(C(C)Nc4ccc(Cl)nc4C4CCNCC4)cc(C)cc3c(=O)n(C)c21]

The titled compound was prepared using similar procedures as described for Example 16 with 1-ethyl-1H-pyrazol-5-amine replacing 1-methyl-1H-pyrazol-5-amine in Step 1 and tert-butyl 4-(3-amino-6-chloro-2-pyridyl)piperidine-1-carboxylate replacing 2-aminobenzoic acid in Step 7. The crude product was dissolved the mixture of DCM/TFA (1:1) and stirred for 30 min. After concentration under the vacuum, the crude product was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₃₂ClN₆O (M+H)⁺ m/z=479.2 found 479.1.

### Step 3: 9-[1-[[2-(1-acetyl-4-piperidyl)-6-chloro-3-pyridyl]amino]ethyl]-3-ethyl-4,7-dimethyl-pyrazolo[3,4-c]isoquinolin-5-one

To a solution of 9-[1-[[6-chloro-2-(4-piperidyl)-3-pyridyl]amino]ethyl]-3-ethyl-4,7-dimethyl-pyrazolo[3,4-c]isoquinolin-5-one (10 mg, 0.02 mmol) in 1 mL of DCM was added N-ethyl-N-isopropyl-propan-2-amine (18 μL, 0.10 mmol). Then acetyl chloride (4 μL, 0.06 mmol) dissolved in 0.5 mL DCM was added dropwise at 0° C. The resulting solution was stirred at 0° C. for 15 min and then concentrated under vacuum. The crude product was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₃₄ClN₆O₂ (M+H)⁺ m/z=521.2; found page 521.2.

### Example 242: 9-(1-((6-Chloro-2-(1-methyl-1H-1,2,4-triazol-3-yl)pyridin-3-yl)amino)ethyl)-3-(2-hydroxyethyl)-4,7-dimethyl-3,4-dihydro-5H-pyrazolo[3,4-c]isoquinolin-5-one

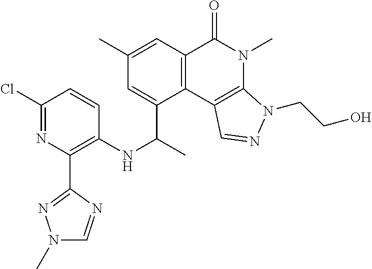 [SMILES: Cc1cc(C(C)Nc2ccc(Cl)nc2-c2ncn(C)n2)c2c(c1)c(=O)n(C)c1c2cnn1CCO]

The titled compound was prepared using similar procedures as described for Example 16 with 1-(2-((triisopropylsilyl)oxy)ethyl)-1H-pyrazol-5-amine (intermediate 51) replacing 1-methyl-1H-pyrazol-5-amine in Step 1, and 6-chloro-2-(1-methyl-1H-1,2,4-triazol-3-yl)pyridin-3-amine replacing 2-aminobenzoic acid in Step 7. The resulting mixture was diluted with 1 mL THF and tetrabutylammonium fluoride (THF solution, 86.44 uL, 0.09 mmol) was added to the reaction mixture at 0° C. The reaction mixture was stirred at 0° C. for 30 min. LCMS analysis indicated the reaction was complete. The reaction was quenched with water and extracted with EtOAc. The combined organics were concentrated under reduced pressure. The residue was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₆ClN₈O2 (M+H)⁺ m/z=493.2; found 493.2.

### Examples 243-252

Examples 243-252 in Table 36 were prepared similarly as described for Example 242.

TABLE 36

| | | | LCMS |
|---|---|---|---|
| Example | | | m/z |
| # | Chemical name | Structure | (M + H)⁺ |
| 243 | 4-(6-chloro-3-((1-(3-(2- hydroxyethyl)-4,7-dimethyl-5- oxo-4,5-dihydro-3H- pyrazolo[3,4-c]isoquinolin-9- yl)ethyl)amino)pyridin-2-yl)-2- fluoro-N-methylbenzamide | 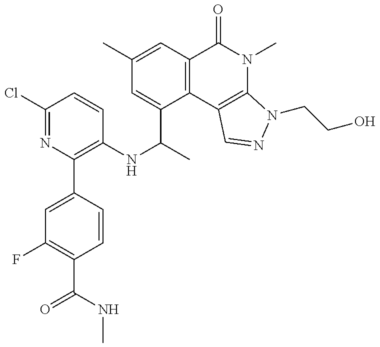 [SMILES: CNC(=O)c1ccc(-c2nc(Cl)ccc2NC(C)c2cc(C)cc3c(=O)n(C)c4c(cnn4CCO)c23)cc1F] | 563.2 |
| 244 | 9-(1-((6-chloro-2-(1-methyl- 1H-pyrazol-4-yl)pyridin-3- yl)amino)ethyl)-3-(2- hydroxyethyl)-4,7-dimethyl- 3,4-dihydro-5H-pyrazolo[3,4- c]isoquinolin-5-one | 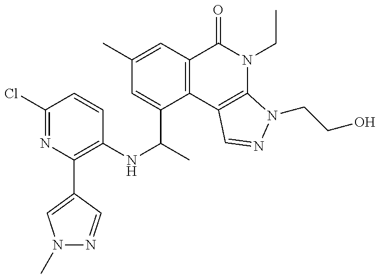 [SMILES: CCn1c(=O)c2cc(C)cc(C(C)Nc3ccc(Cl)nc3-c3cnn(C)c3)c2c2cnn(CCO)c21] | 492.2 |
| 245 | 9-(1-((6-chloro-2-(4- hydroxypiperidin-1-yl)pyridin- 3-yl)amino)ethyl)-3-(2- hydroxyethyl)-4,7-dimethyl- 3,4-dihydro-5H-pyrazolo[3,4- c]isoquinolin-5-one | 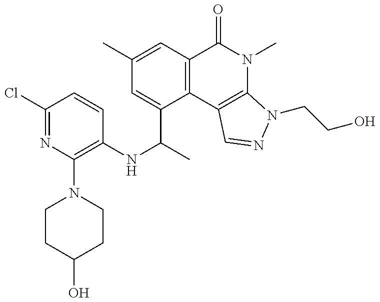 [SMILES: Cc1cc(C(C)Nc2ccc(Cl)nc2N2CCC(O)CC2)c2c(c1)c(=O)n(C)c1c2cnn1CCO] | 511.2 |
| 246 | 9-(1-((6-chloro-2-(quinolin-6- yl)pyridin-3-yl)amino)ethyl)-3- (2-hydroxyethyl)-4,7- dimethyl-3,4-dihydro-5H- pyrazolo[3,4-c]isoquinolin-5- one | 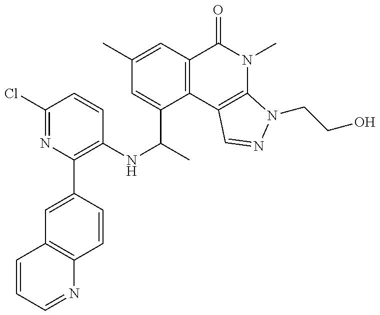 [SMILES: Cc1cc(C(C)Nc2ccc(Cl)nc2-c2ccc3ncccc3c2)c2c(c1)c(=O)n(C)c1c2cnn1CCO] | 539.2 |
| 247 | 9-(1-((6-chloro-2-(1-methyl- 1H-indazol-5-yl)pyridin-3- yl)amino)ethyl)-3-(2- hydroxyethyl)-4,7-dimethyl- 3,4-dihydro-5H-pyrazolo[3,4- c]isoquinolin-5-one | 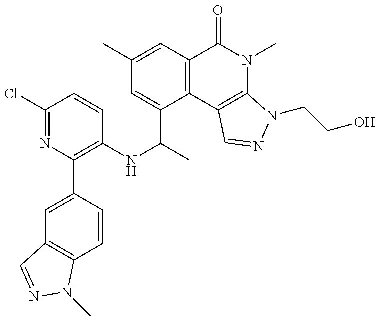 [SMILES: Cc1cc(C(C)Nc2ccc(Cl)nc2-c2ccc3c(cnn3C)c2)c2c(c1)c(=O)n(C)c1c2cnn1CCO] | 542.2 |
| 248 | 9-(1-((6-chloro-2-(2-methyl-1- oxoisoindolin-5-yl)pyridin-3- yl)amino)ethyl)-3-(2- hydroxyethyl)-4,7-dimethyl- 3,4-dihydro-5H-pyrazolo[3,4- c]isoquinolin-5-one | 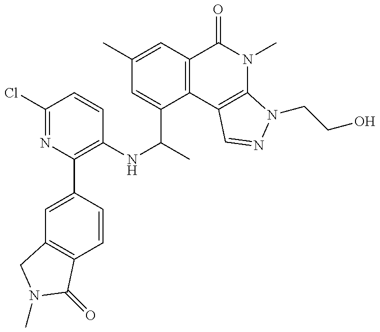 [SMILES: Cc1cc(C(C)Nc2ccc(Cl)nc2-c2ccc3c(c2)CN(C)C3=O)c2c(c1)c(=O)n(C)c1c2cnn1CCO] | 557.2 |
| 249 | 3-(2-hydroxyethyl)-4,7- dimethyl-9-(1-((6-methyl-2-(1- methyl-1H-pyrazol-4- yl)pyridin-3-yl)amino)ethyl)- 3,4-dihydro-5H-pyrazolo[3,4- c]isoquinolin-5-one | 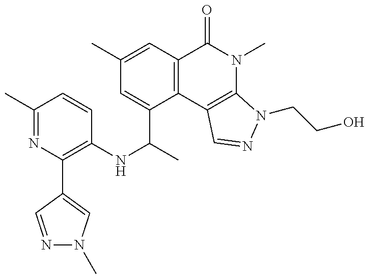 [SMILES: Cc1cc(C(C)Nc2ccc(C)nc2-c2cnn(C)c2)c2c(c1)c(=O)n(C)c1c2cnn1CCO] | 472.2 |
| 250 | 9-(1-((6-chloro-2-(1,3,4- oxadiazol-2-yl)pyridin-3- yl)amino)ethyl)-3-(2- hydroxyethyl)-4,7-dimethyl- 3,4-dihydro-5H-pyrazolo[3,4- c]isoquinolin-5-one | 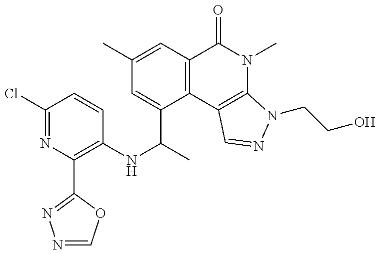 [SMILES: Cc1cc(C(C)Nc2ccc(Cl)nc2-c2nnco2)c2c(c1)c(=O)n(C)c1c2cnn1CCO] | 480.1 |
| 251 | 9-(1-((6-chloro-2-(2-methyl- 2H-tetrazol-5-yl)pyridin-3- yl)amino)ethyl)-3-(2- hydroxyethyl)-4,7-dimethyl- 3,4-dihydro-5H-pyrazolo[3,4- c]isoquinolin-5-one | 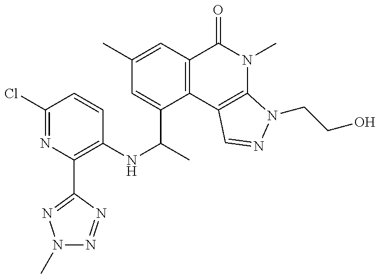 [SMILES: Cc1cc(C(C)Nc2ccc(Cl)nc2-c2nnn(C)n2)c2c(c1)c(=O)n(C)c1c2cnn1CCO] | 494.2 |
| 252 | 9-(1-((6-chloro-2-(pyrimidin- 5-yl)pyridin-3-yl)amino)ethyl)- 3-(2-hydroxyethyl)-4,7- dimethyl-3,4-dihydro-5H- pyrazolo[3,4-c]isoquinolin-5- one | 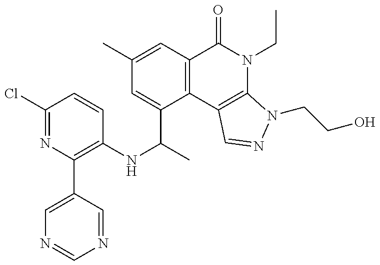 [SMILES: CCn1c(=O)c2cc(C)cc(C(C)Nc3ccc(Cl)nc3-c3cncnc3)c2c2cnn(CCO)c21] | 490.2 |

### Example 253. 5-[6-Chloro-3-[1-[3-(2-hydroxyethyl)-4,7-dimethyl-5-oxo-pyrazolo[3,4-c]isoquinolin-9-yl]ethylamino]-2-pyridyl]-N-methyl-pyridine-2-carboxamide

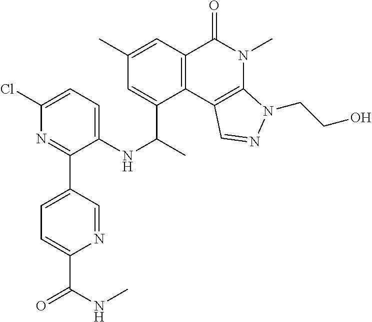 [SMILES: CNC(=O)c1ccc(-c2nc(Cl)ccc2NC(C)c2cc(C)cc3c(=O)n(C)c4c(cnn4CCO)c23)cn1]

### Step 1: 9-[1-[(2-bromo-6-chloro-3-pyridyl)amino]ethyl]-3-(2-hydroxyethyl)-4,7-dimethyl-pyrazolo[3,4-c]isoquinolin-5-one

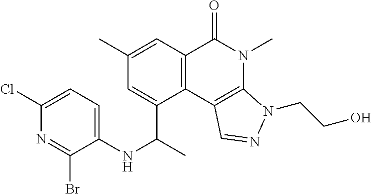 [SMILES: Cc1cc(C(C)Nc2ccc(Cl)nc2Br)c2c(c1)c(=O)n(C)c1c2cnn1CCO]

The titled compound was prepared using similar procedures as described for Example 16 with 1-(2-((triisopropylsilyl)oxy)ethyl)-1H-pyrazol-5-amine (Intermediate 51) replacing 1-methyl-1H-pyrazol-5-amine in Step 1, and 2-bromo-6-chloro-pyridin-3-amine replacing 2-aminobenzoic acid in Step 7. The resulting mixture was diluted with 1 mL THF and tetrabutylammonium fluoride (THF solution, 86.44 μL, 0.09 mmol) was added to the reaction mixture at 0° C. The reaction was stirred at 0° C. for 30 min then quenched with water and extracted with EtOAc. The combined organics were concentrated under reduced pressure. The residue was purified by silica gel column chromatography, eluted with DCM and MeOH to provide the desired product. LCMS calculated for C₂₁H₂₂BrClN₅O₂ (M+H)⁺ m/z=490.1 found 490.0.

### Step 2: 5-[6-chloro-3-[1-[3-(2-hydroxyethyl)-4,7-dimethyl-5-oxo-pyrazolo[3,4-c]isoquinolin-9-yl]ethylamino]-2-pyridyl]-N-methyl-pyridine-2-carboxamide

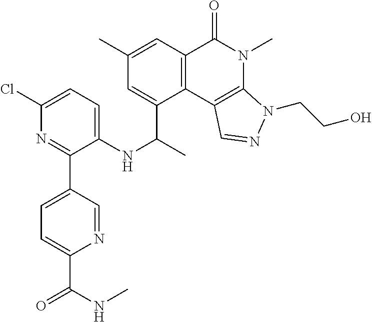 [SMILES: CNC(=O)c1ccc(-c2nc(Cl)ccc2NC(C)c2cc(C)cc3c(=O)n(C)c4c(cnn4CCO)c23)cn1]

A mixture of 9-[1-[(2-bromo-6-chloro-3-pyridyl)amino]ethyl]-3-(2-hydroxyethyl)-4,7-dimethyl-pyrazolo[3,4-c]isoquinolin-5-one (12.5 mg, 0.03 mmol), tetrakis(triphenylphosphine)palladium(0) (5.9 mg, 0.01 mmol), K₃PO₄ (16.2 mg, 0.08 mmol) and N-methyl-5-(4,4,5,5-tetramethyl-1,3,2-dioxaborolan-2-yl)pyridine-2-carboxamide (8.01 mg, 0.03 mmol) in 1 mL dioxane/H₂O(5:1) was bubbled with N₂ for 1 min. The reaction mixture was stirred at 80° C. for 12 h under nitrogen atmosphere. LCMS analysis indicated the reaction was complete. The reaction was quenched with water and extracted with EA. The combined organics were concentrated under reduced pressure. The residue was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a light yellow solid. LCMS calculated for C₂₈H₂₉ClN₇O₃ (M+H)⁺ m/z=546.2; found 546.2.

### Examples 254-256

Examples 254-256 in Table 37 were prepared similarly as described for Example 253.

TABLE 37

| | | | LCMS |
|---|---|---|---|
| Example | | | m/z |
| # | Chemical name | Structure | (M + H)⁺ |
| 254 | 9-[1-[[6-chloro-2-(1-methyl-6- oxo-3-pyridyl)-3- pyridyl]amino]ethyl]-3-(2- hydroxyethyl)-4,7-dimethyl- pyrazolo[3,4-c]isoquinolin-5- one | 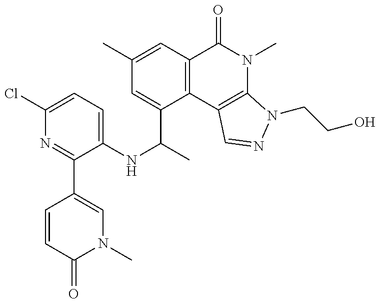 [SMILES: Cc1cc(C(C)Nc2ccc(Cl)nc2-c2ccc(=O)n(C)c2)c2c(c1)c(=O)n(C)c1c2cnn1CCO] | 519.2 |
| 255 | 9-[1-[[6-chloro-2-(1-methyl-2- oxo-4-pyridyl)-3- pyridyl]amino]ethyl]-3-(2- hydroxyethyl)-4,7-dimethyl- pyrazolo[3,4-c]isoquinolin-5- one | 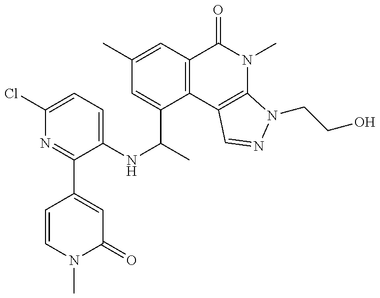 [SMILES: Cc1cc(C(C)Nc2ccc(Cl)nc2-c2ccn(C)c(=O)c2)c2c(c1)c(=O)n(C)c1c2cnn1CCO] | 519.2 |
| 256 | 9-[1-[[6-chloro-2-(2-methoxy-4- pyridyl)-3- pyridyl]amino]ethyl]-3-(2- hydroxyethyl)-4,7-dimethyl- pyrazolo[3,4-c]isoquinolin-5- one | 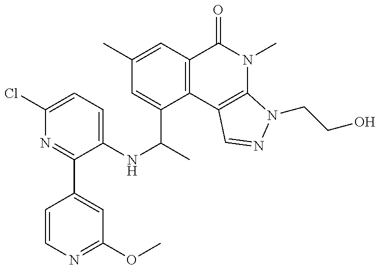 [SMILES: COc1cc(-c2nc(Cl)ccc2NC(C)c2cc(C)cc3c(=O)n(C)c4c(cnn4CCO)c23)ccn1] | 519.2 |

### Example 257. 9-[1-[[6-Chloro-2-(1-methyl-4-piperidyl)-3-pyridyl]amino]ethyl]-3-(2-hydroxyethyl)-4,7-dimethyl-pyrazolo[3,4-c]isoquinolin-5-one

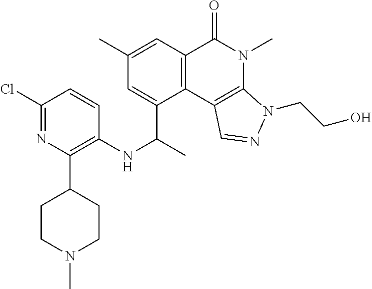 [SMILES: Cc1cc(C(C)Nc2ccc(Cl)nc2C2CCN(C)CC2)c2c(c1)c(=O)n(C)c1c2cnn1CCO]

### Step 1: 9-[1-[[6-chloro-2-(4-piperidyl)-3-pyridyl]amino]ethyl]-3-(2-hydroxyethyl)-4,7-dimethyl-pyrazolo[3,4-c]isoquinolin-5-one

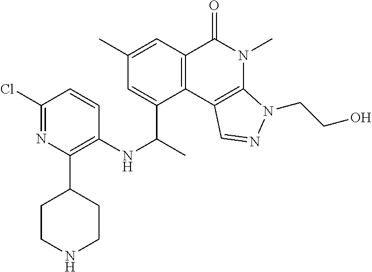 [SMILES: Cc1cc(C(C)Nc2ccc(Cl)nc2C2CCNCC2)c2c(c1)c(=O)n(C)c1c2cnn1CCO]

The titled compound was prepared using similar procedures as described for Example 16 with 1-(2-((triisopropylsilyl)oxy)ethyl)-1H-pyrazol-5-amine (Intermediate 51) replacing 1-methyl-1H-pyrazol-5-amine in Step 1, and tert-butyl 4-(3-amino-6-chloro-2-pyridyl)piperidine-1-carboxylate (Example 241, step 1) replacing 2-aminobenzoic acid in Step 7. The reaction was quenched with 1 mL water and extracted with 2×1 mL EA. The organics were concentrated to dryness. The residue was dissolved in 1 mL 4N HCl in dioxane and the resulting mixture was kept stirred at rt for 2 h. LCMS analysis indicated the reaction was complete. The crude was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product (29.00 mg, 61.0%). LCMS calculated for C₂₆H₃₂ClN₆O₂ (M+H)⁺ m/z=495.2; found 495.2.

### Step 2: 9-[1-[[6-chloro-2-(1-methyl-4-piperidyl)-3-pyridyl]amino]ethyl]-3-(2-hydroxyethyl)-4,7-dimethyl-pyrazolo[3,4-c]isoquinolin-5-one

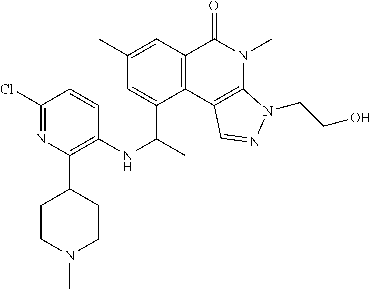 [SMILES: Cc1cc(C(C)Nc2ccc(Cl)nc2C2CCN(C)CC2)c2c(c1)c(=O)n(C)c1c2cnn1CCO]

To a stirred solution of 9-[1-[[6-chloro-2-(4-piperidyl)-3-pyridyl]amino]ethyl]-3-(2-hydroxyethyl)-4,7-dimethyl-pyrazolo[3,4-c]isoquinolin-5-one (7.00 mg, 0.01 mmol) in 0.5 mL DCM was added formaldehyde (5.74 μL, 0.07 mmol) and the resulting mixture was kept stirred at rt for 15 min. Sodium triacetoxyborohydride (8.99 mg, 0.04 mmol) was then added to the reaction mixture and the resulting mixture was kept stirred at rt for 1 h. LCMS analysis indicated the reaction was complete. The crude was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product. LCMS calculated for C₂₇H₃₄ClN₆O₂ (M+H)⁺ m/z=509.2. found: 509.2.

### Example 258. 9-[1-[[2-(1-Acetyl-4-piperidyl)-6-chloro-3-pyridyl]amino]ethyl]-3-(2-hydroxyethyl)-4,7-dimethyl-pyrazolo[3,4-c]isoquinolin-5-one

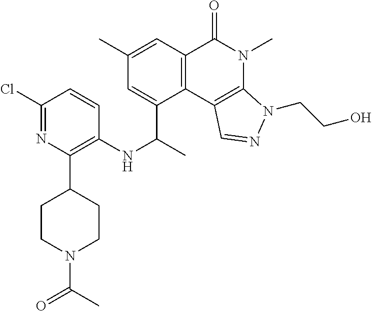 [SMILES: CC(=O)N1CCC(c2nc(Cl)ccc2NC(C)c2cc(C)cc3c(=O)n(C)c4c(cnn4CCO)c23)CC1]

To a stirred solution of 9-[1-[[6-chloro-2-(4-piperidyl)-3-pyridyl]amino]ethyl]-3-(2-hydroxyethyl)-4,7-dimethyl-pyrazolo[3,4-c]isoquinolin-5-one (Example 257, step 1) (7.00 mg, 0.01 mmol) and acetyl chloride (1.47 μL, 0.02 mmol) in 1 mL DCM was added N-ethyl-N-isopropyl-propan-2-amine (7.6 μL, 0.04 mmol) and the resulting mixture was kept stirred at rt for 1 h. LCMS analysis indicated the reaction was complete. The crude was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product. LCMS calculated for C₂₈H₃₄ClN₆O₃ (M+H)⁺ m/z=537.2. found: 537.2.

### Example 259. 9-[1-[[6-Chloro-2-(1-methyl-1,2,4-triazol-3-yl)-3-pyridyl]amino]ethyl]-4-(2,2-difluoroethyl)-3-(2-hydroxyethyl)-7-methyl-pyrazolo[3,4-c]isoquinolin-5-one

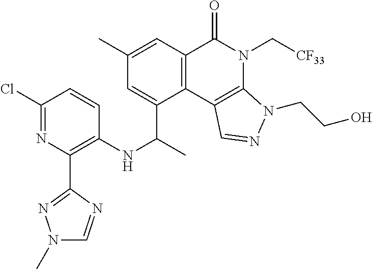

The titled compound was prepared using similar procedures as described for Example 16 with 1-(2-((triisopropylsilyl)oxy)ethyl)-1H-pyrazol-5-amine (intermediate 51) replacing 1-methyl-1H-pyrazol-5-amine in Step 1, 1,1-difluoro-2-iodoethane replacing iodomethane in Step 2, and 6-chloro-2-(1-methyl-1H-1,2,4-triazol-3-yl)pyridin-3-amine replacing 2-aminobenzoic acid in Step 7. The product was deprotected with TBAF. The crude product was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₂₆ClF₂N₈O₂ (M+H)⁺ m/z=543.2; found 543.2.

### Example 260. 5-[6-Chloro-3-[1-[4-(2,2-difluoroethyl)-3-(2-hydroxyethyl)-7-methyl-5-oxo-pyrazolo[3,4-c]isoquinolin-9-yl]ethylamino]-2-pyridyl]-N-methyl-pyridine-2-carboxamide

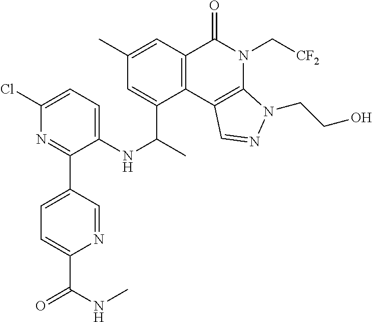

### Step 1: 9-[1-[(2-bromo-6-chloro-3-pyridyl)amino]ethyl]-4-(2,2-difluoroethyl)-3-(2-hydroxyethyl)-7-methyl-pyrazolo[3,4-c]isoquinolin-5-one

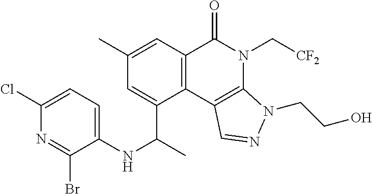

The titled compound was prepared using similar procedures as described for Example 16 with 1-(2-((triisopropylsilyl)oxy)ethyl)-1H-pyrazol-5-amine (intermediate 51) replacing 1-methyl-1H-pyrazol-5-amine in Step 1, 1,1-difluoro-2-iodoethane replacing iodomethane in Step 2, and 2-bromo-6-chloropyridin-3-amine replacing 2-aminobenzoic acid in Step 7. The product was deprotected with TBAF. The crude product was purified by silica gel column chromatography, eluted with DCM and MeOH to provide the desired product (49.0 mg, 79.0%) as a white solid. LCMS calculated for C₂₂H₂₃BrClF₂N₅O₂ (M+H)⁺ m/z=540.1; found 540.1.

### Step 2: 5-[6-chloro-3-[1-[4-(2,2-difluoroethyl)-3-(2-hydroxyethyl)-7-methyl-5-oxo-pyrazolo[3,4-c]isoquinolin-9-yl]ethylamino]-2-pyridyl]-N-methyl-pyridine-2-carboxamide

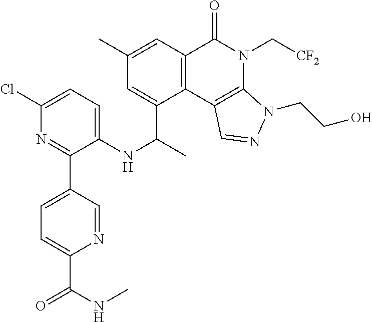

A mixture of 9-[1-[(2-bromo-6-chloro-3-pyridyl)amino]ethyl]-4-(2,2-difluoroethyl)-3-(2-hydroxyethyl)-7-methyl-pyrazolo[3,4-c]isoquinolin-5-one (15.00 mg, 0.03 mmol), tetrakis(triphenylphosphine)palladium(0) (6.41 mg, 0.01 mmol), tripotassium phosphate (17.66 mg, 0.08 mmol) and N-methyl-5-(4,4,5,5-tetramethyl-1,3,2-dioxaborolan-2-yl)pyridine-2-carboxamide (10.91 mg, 0.04 mmol) in 1 mL dioxane/H₂O(5:1) was bubbled with N₂ for 1 min. The reaction mixture was stirred at 80° C. for 12 h under nitrogen atmosphere. LCMS analysis indicated the reaction was complete. The reaction was quenched with water and extracted with EA. The combined organics were concentrated under reduced pressure. The residue was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a light yellow solid. LCMS calculated for C₂₉H₂₉ClF₂N₇O₃ (M+H)⁺ m/z=596.2; found 596.2.

### Example 261. 9-[1-[[6-Chloro-2-(1-methyl-1,2,4-triazol-3-yl)-3-pyridyl]amino]ethyl]-4-ethyl-3-(2-hydroxyethyl)-7-methyl-pyrazolo[3,4-c]isoquinolin-5-one

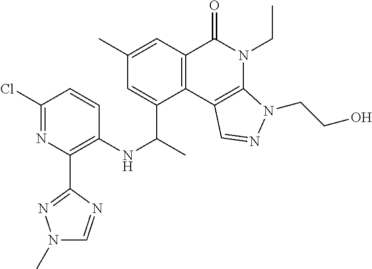 [SMILES: CCn1c(=O)c2cc(C)cc(C(C)Nc3ccc(Cl)nc3-c3ncn(C)n3)c2c2cnn(CCO)c21]

The titled compound was prepared using similar procedures as described for Example 16 with 1-(2-((triisopropylsilyl)oxy)ethyl)-1H-pyrazol-5-amine (intermediate 51) replacing 1-methyl-1H-pyrazol-5-amine in Step 1, iodoethane replacing iodomethane in Step 2, and 6-chloro-2-(1-methyl-1H-1,2,4-triazol-3-yl)pyridin-3-amine replacing 2-aminobenzoic acid in Step 7. The product was deprotected with TBAF. The crude product was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₂₈ClN₈O₂ (M+H)⁺ m/z=507.2; found 507.2.

### Example 262. 9-[1-[(6-Chloro-2-morpholino-3-pyridyl)amino]ethyl]-4-ethyl-3-(2-hydroxyethyl)-7-methyl-pyrazolo[3,4-c]isoquinolin-5-one

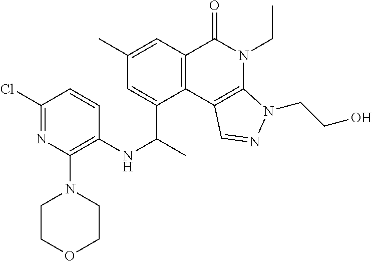 [SMILES: CCn1c(=O)c2cc(C)cc(C(C)Nc3ccc(Cl)nc3N3CCOCC3)c2c2cnn(CCO)c21]

The titled compound was prepared using similar procedures as described for Example 16 with 1-(2-((triisopropylsilyl)oxy)ethyl)-1H-pyrazol-5-amine (intermediate 51) replacing 1-methyl-1H-pyrazol-5-amine in Step 1, iodoethane replacing iodomethane in Step 2, and 6-chloro-2-morpholinopyridin-3-amine replacing 2-aminobenzoic acid in Step 7. The product was deprotected with TBAF. The crude product was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₃₂ClN₆O₃ (M+H)⁺ m/z=511.2; found 511.2.

### Example 263. 9-[1-[[6-Chloro-2-(1-methylpyrazol-4-yl)-3-pyridyl]amino]ethyl]-4,7-dimethyl-3-(2-morpholinoethyl)pyrazolo[3,4-c]isoquinolin-5-one

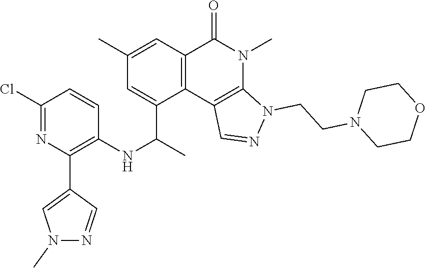 [SMILES: Cc1cc(C(C)Nc2ccc(Cl)nc2-c2cnn(C)c2)c2c(c1)c(=O)n(C)c1c2cnn1CCN1CCOCC1]

To a stirred solution of 9-[1-[[6-chloro-2-(1-methylpyrazol-4-yl)-3-pyridyl]amino]ethyl]-3-(2-hydroxyethyl)-4,7-dimethyl-pyrazolo[3,4-c]isoquinolin-5-one (Example 244) (35 mg, 0.07 mmol), methanesulfonyl chloride (8 uL, 12.22 mg, 0.11 mmol) in 2 mL DCM was added N,N-diethylethanamine (29.75 uL, 0.21 mmol) and the resulting mixture was kept stirred at rt for 2 h. The reaction mixture was then concentrated to dryness under reduced pressure. A solution of the residue in 0.2 mL DMF was added morpholine (11 mg, 0.13 mmol) and the resulting mixture was kept stirred at rt for 4 h. LCMS analysis indicated the reaction was complete. The reaction mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₄ClN₈O₂ (M+H)⁺ m/z=561.2; found 561.2.

### Examples 264-268

Examples 264-268 in Table 38 were prepared similarly as described for Example 263.

TABLE 38

| | | | LCMS |
|---|---|---|---|
| Example | | | m/z |
| # | Chemical name | Structure | (M + H)⁺ |
| 264 | 9-(1-((6-chloro-3′-fluoro- [2,4′-bipyridin]-3- yl)amino)ethyl)-4,7- dimethyl-3-(2- morpholinoethyl)-3,4- dihydro-5H-pyrazolo[3,4- c]isoquinolin-5-one | 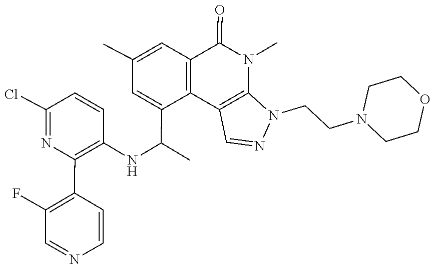 [SMILES: Cc1cc(C(C)Nc2ccc(Cl)nc2-c2ccncc2F)c2c(c1)c(=O)n(C)c1c2cnn1CCN1CCOCC1] | 576.2 |
| 265 | 9-(1-((6-chloro-3′-fluoro- [2,4′-bipyridin]-3- yl)amino)ethyl)-4,7- dimethyl-3-(2-(4- methylpiperazin-1- yl)ethyl)-3,4-dihydro-5H- pyrazolo[3,4- c]isoquinolin-5-one | 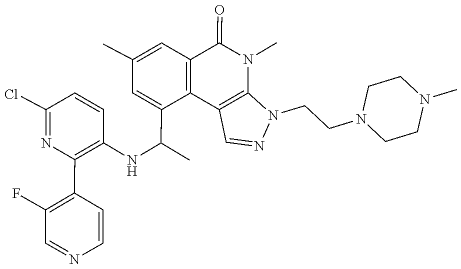 [SMILES: Cc1cc(C(C)Nc2ccc(Cl)nc2-c2ccncc2F)c2c(c1)c(=O)n(C)c1c2cnn1CCN1CCN(C)CC1] | 589.3 |
| 266 | 4-(6-chloro-3-((1-(4,7- dimethyl-3-(2- morpholinoethyl)-5-oxo- 4,5-dihydro-3H- pyrazolo[3,4- c]isoquinolin-9- yl)ethyl)amino)pyridin-2- yl)-2-fluoro-N- methylbenzamide | 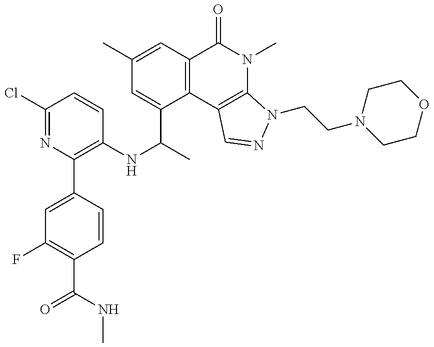 [SMILES: CNC(=O)c1ccc(-c2nc(Cl)ccc2NC(C)c2cc(C)cc3c(=O)n(C)c4c(cnn4CCN4CCOCC4)c23)cc1F] | 632.3 |
| 267 | 4-(6-chloro-3-((1-(4,7- dimethyl-3-(2-(4- methylpiperazin-1- yl)ethyl)-5-oxo-4,5- dihydro-3H-pyrazolo[3,4- c]isoquinolin-9- yl)ethyl)amino)pyridin-2- yl)-2-fluoro-N- methylbenzamide | 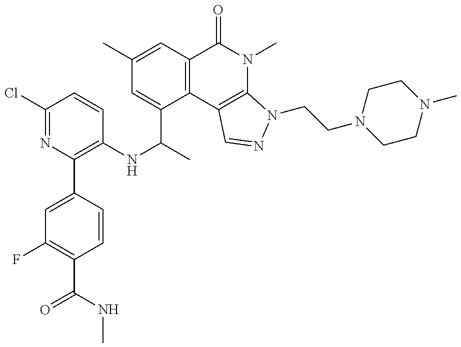 [SMILES: CNC(=O)c1ccc(-c2nc(Cl)ccc2NC(C)c2cc(C)cc3c(=O)n(C)c4c(cnn4CCN4CCN(C)CC4)c23)cc1F] | 645.3 |
| 268 | 9-(1-((6-chloro-2-(1- methyl-1H-1,2,4-triazol-3- yl)pyridin-3- yl)amino)ethyl)-4-(2,2- difluoroethyl)-3-(2-(5,6- dihydro- [1,2,4]triazolo[1,5- a]pyrazin-7(8H)-yl)ethyl)- 7-methyl-3,4-dihydro-5H- pyrazolo[3,4- c]isoquinolin-5-one | 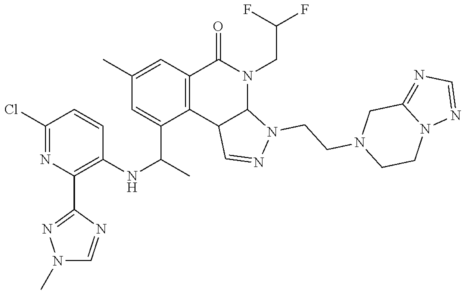 [SMILES: Cc1cc2c(c(C(C)Nc3ccc(Cl)nc3-c3ncn(C)n3)c1)C1C=NN(CCN3CCn4ncnc4C3)C1N(CC(F)F)C2=O] | 649.2 |

### Example 269: 9-[1-[[6-Chloro-2-(1-methyl-1,2,4-triazol-3-yl)-3-pyridyl]amino]ethyl]-4-ethyl-7-methyl-3-(2-pyridyl)pyrazolo[3,4-c]isoquinolin-5-one

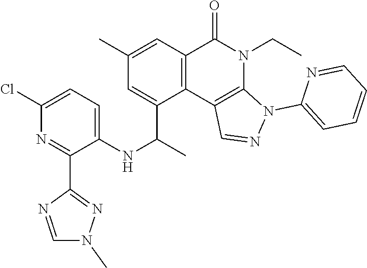 [SMILES: CCn1c(=O)c2cc(C)cc(C(C)Nc3ccc(Cl)nc3-c3ncn(C)n3)c2c2cnn(-c3ccccn3)c21]

### Step 1:9-(1-bromoethyl)-4-ethyl-7-methyl-3-(2-pyridyl)pyrazolo[3,4-c]isoquinolin-5-one

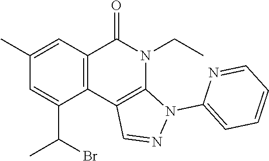 [SMILES: CCn1c(=O)c2cc(C)cc(C(C)Br)c2c2cnn(-c3ccccn3)c21]

The title compound was prepared using similar procedures as described for Example 81 with ethyl 5-amino-1-(2-pyridyl) pyrazole-4-carboxylate replacing ethyl 5-amino-1-(1-(tert-butoxycarbonyl) azetidin-3-yl)-1H-pyrazole-4-carboxylate in Step 3, and iodoethane replacing MeI in step 5. The residue was used directly in the next step without further purification.

### Step 2: 9-[1-[[6-chloro-2-(1-methyl-1,2,4-triazol-3-yl)-3-pyridyl]amino]ethyl]-4-ethyl-7-methyl-3-(2-pyridyl)pyrazolo[3,4-c]isoquinolin-5-one

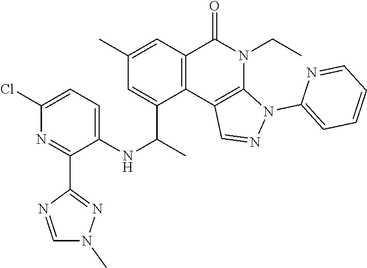 [SMILES: CCn1c(=O)c2cc(C)cc(C(C)Nc3ccc(Cl)nc3-c3ncn(C)n3)c2c2cnn(-c3ccccn3)c21]

To a stirred mixture of 9-(1-bromoethyl)-4-ethyl-7-methyl-3-(2-pyridyl)pyrazolo[3,4-c]isoquinolin-5-one (10 mg, 0.02 mmol) in 0.1 mL DMF was added 6-chloro-2-(1-methyl-1,2,4-triazol-3-yl)pyridin-3-amine (10 mg, 0.05 mmol). The mixture was stirred at 65° C. for 2 h. The residue was diluted with MeOH and then purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product (3.40 mg, 25.9%) as a white solid. LCMS calculated for C₂₈H₂₇ClN₉O (M+H)⁺ m/z=540.2; found 540.1.

### Examples 270-273

Examples 270-273 in Table 39 were prepared similarly as described for Example 269.

TABLE 39

| | | | LCMS |
|---|---|---|---|
| Example | | | m/z |
| # | Chemical name | Structure | (M + H)⁺ |
| 270 | 9-(1-((6-chloro-2- morpholinopyridin-3- yl)amino)ethyl)-4-ethyl-7- methyl-3-(pyridin-2-yl)-3,4- dihydro-5H-pyrazolo[3,4- c]isoquinolin-5-one | 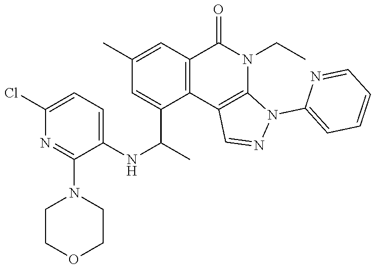 [SMILES: CCn1c(=O)c2cc(C)cc(C(C)Nc3ccc(Cl)nc3N3CCOCC3)c2c2cnn(-c3ccccn3)c21] | 544.2 |
| 271 | 6-chloro-3-((1-(4-ethyl-7- methyl-5-oxo-3-(pyridin-2-yl)- 4,5-dihydro-3H-pyrazolo[3,4- c]isoquinolin-9-yl)ethyl)amino)- N-methyl-[2,3′-bipyridine]-6′- carboxamide | 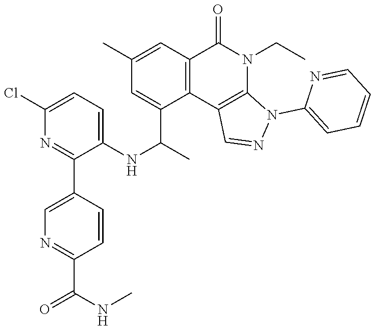 [SMILES: CCn1c(=O)c2cc(C)cc(C(C)Nc3ccc(Cl)nc3-c3ccc(C(=O)NC)nc3)c2c2cnn(-c3ccccn3)c21] | 593.1 |
| 272 | 3-(6-chloro-3-((1-(4,7-dimethyl- 5-oxo-3-(pyridin-2-yl)-4,5- dihydro-3H-pyrazolo[3,4- c]isoquinolin-9- yl)ethyl)amino)pyridin-2-yl)- 1,2,4-oxadiazol-5(4H)-one | 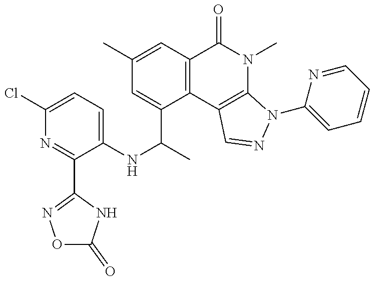 [SMILES: Cc1cc(C(C)Nc2ccc(Cl)nc2-c2noc(=O)[nH]2)c2c(c1)c(=O)n(C)c1c2cnn1-c1ccccn1] | 529.1 |
| 273 | 9-(1-((6-chloro-2-(1-methyl-1H- pyrazol-4-yl)pyridin-3- yl)amino)ethyl)-4,7-dimethyl-3- (pyridin-2-yl)-3,4-dihydro-5H- pyrazolo[3,4-c]isoquinolin-5- one | 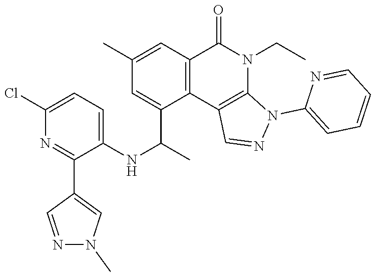 [SMILES: CCn1c(=O)c2cc(C)cc(C(C)Nc3ccc(Cl)nc3-c3cnn(C)c3)c2c2cnn(-c3ccccn3)c21] | 525.1 |

### Example 274: 9-[1-[[6-Chloro-2-(1-methyl-1,2,4-triazol-3-yl)-3-pyridyl]amino]ethyl]-4-ethyl-7-methyl-3-(3-pyridyl)pyrazolo[3,4-c]isoquinolin-5-one

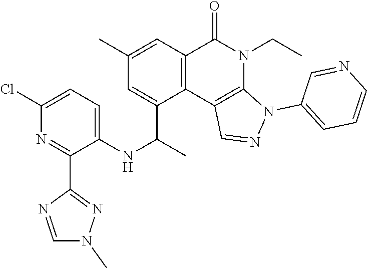 [SMILES: CCn1c(=O)c2cc(C)cc(C(C)Nc3ccc(Cl)nc3-c3ncn(C)n3)c2c2cnn(-c3cccnc3)c21]

The titled compound was prepared using similar procedures as described for Example 81 with 3-pyridylhydrazine replacing tert-butyl 3-hydrazineylazetidine-1-carboxylate in Step 2, iodoethane replacing iodomethane in step 5, and 6-chloro-2-(1-methyl-1H-1,2,4-triazol-3-yl)pyridin-3-amine replacing 6-chloro-2-(1-methyl-1H-pyrazol-4-yl)pyridin-3-amine in step 10. The crude product A: water (0.1% TFA), mobile phase B: acetonitrile; flow rate: 60 mL/min); eluted fractions were collected and lyophilized to provide the TFA salt of the desired product as a white solid. LCMS calculated for C₂₈H₂₇ClN₉O (M+H)⁺ m/z=540.2; found 540.1.

### Example 275: 9-[1-[[6-Chloro-2-(1-methylpyrazol-4-yl)-3-pyridyl]amino]ethyl]-4,7-dimethyl-3-(2-pyridylmethyl)pyrazolo[3,4-c]isoquinolin-5-one

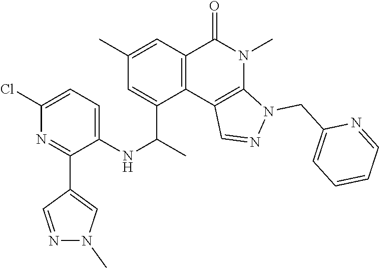 [SMILES: Cc1cc(C(C)Nc2ccc(Cl)nc2-c2cnn(C)c2)c2c(c1)c(=O)n(C)c1c2cnn1Cc1ccccn1]

The titled compound was prepared using similar procedures as described for Example 81 with 2-(hydrazineylmethyl)pyridine replacing tert-butyl 3-hydrazineylazetidine-1-carboxylate in Step 2. The crude product A: water (0.1% TFA), mobile phase B: acetonitrile; flow rate: 60 mL/min); eluted fractions were collected and lyophilized to provide the TFA salt of the desired product as a white solid. LCMS calculated for C₂₉H₂₈ClN₈O (M+H)⁺ m/z=539.2; found 539.1.

### Example 276. 3-(6-Chloro-3-((1-(4,7-dimethyl-5-oxo-3-(pyridin-2-ylmethyl)-4,5-dihydro-3H-pyrazolo[3,4-c]isoquinolin-9-yl)ethyl)amino)pyridin-2-yl)-1,2,4-oxadiazol-5(4H)-one

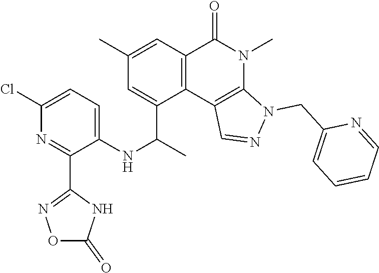 [SMILES: Cc1cc(C(C)Nc2ccc(Cl)nc2-c2noc(=O)[nH]2)c2c(c1)c(=O)n(C)c1c2cnn1Cc1ccccn1]

The titled compound was prepared using similar procedures as described for Example 81 with 2-(hydrazineylmethyl)pyridine replacing tert-butyl 3-hydrazineylazetidine-1-carboxylate in Step 2, and 3-(3-amino-6-chloropyridin-2-yl)-1,2,4-oxadiazol-5(4H)-one replacing 6-chloro-2-(1-methyl-1H-pyrazol-4-yl)pyridin-3-amine in step 10 The crude product A: water (0.1% TFA), mobile phase B: acetonitrile; flow rate: 60 mL/min); eluted fractions were collected and lyophilized to provide the TFA salt of the desired product as a white solid. LCMS calculated for C₂₇H₂₄ClN₈O₃ (M+H)⁺ m/z=543.2; found 543.1.

### Example 277: 9-[1-[[6-Chloro-2-(1-methyl-1,2,4-triazol-3-yl)-3-pyridyl]amino]ethyl]-4,7-dimethyl-3-(1-methyl-3-piperidyl)pyrazolo[3,4-c]isoquinolin-5-one

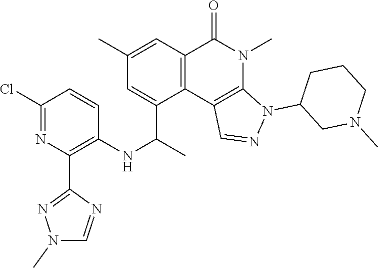 [SMILES: Cc1cc(C(C)Nc2ccc(Cl)nc2-c2ncn(C)n2)c2c(c1)c(=O)n(C)c1c2cnn1C1CCCN(C)C1]

### Step 1: benzyl 3-[9-(1-bromoethyl)-4,7-dimethyl-5-oxo-pyrazolo[3,4-c]isoquinolin-3-yl]piperidine-1-carboxylate

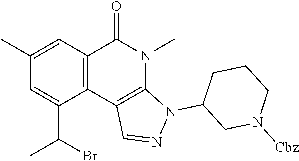 [SMILES: Cc1cc(C(C)Br)c2c(c1)c(=O)n(C)c1c2cnn1C1CCCN(C(=O)OCc2ccccc2)C1]

The titled compound was prepared using similar procedures as described for Example 81 with benzyl 3-hydrazinopiperidine-1-carboxylate replacing tert-butyl 3-hydrazineylazetidine-1-carboxylate in Step 2. The crude product was used directly in next step without further purification.

### Step 2: 9-[1-[[6-chloro-2-(1-methyl-1,2,4-triazol-3-yl)-3-pyridyl]amino]ethyl]-4,7-dimethyl-3-(3-piperidyl)pyrazolo[3,4-c]isoquinolin-5-one

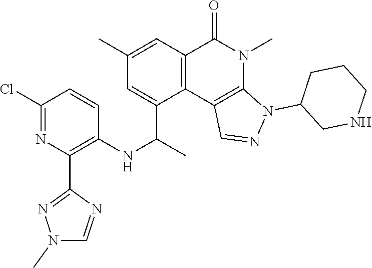 [SMILES: Cc1cc(C(C)Nc2ccc(Cl)nc2-c2ncn(C)n2)c2c(c1)c(=O)n(C)c1c2cnn1C1CCCNC1]

To a stirred solution of benzyl 3-[9-(1-bromoethyl)-4,7-dimethyl-5-oxo-pyrazolo[3,4-c]isoquinolin-3-yl]piperidine-1-carboxylate (80.00 mg, 0.15 mmol) in 0.3 mL DMF was added 6-chloro-2-(1-methyl-1,2,4-triazol-3-yl)pyridin-3-amine (46.81 mg, 0.22 mmol) and the resulting mixture was kept stirred at 65° C. for 4 h. The reaction was quenched with water and extracted with EA. The crude was concentrated and 1 mL HBr in acetic acid (33 wt. %) was added at 0° C. The resulting mixture kept stirred at 0° C. for 15 min. The reaction mixture was diluted with MeOH, and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product (42.00 mg, 53.0%) as a white solid. LCMS calculated for C₂₇H₃₁ClN₉O (M+H)⁺ m/z=532.2; found 532.2.

### Step 3: 9-[1-[[6-chloro-2-(1-methyl-1,2,4-triazol-3-yl)-3-pyridyl]amino]ethyl]-4,7-dimethyl-3-(1-methyl-3-piperidyl)pyrazolo[3,4-c]isoquinolin-5-one

To a stirred solution of 9-[1-[[6-chloro-2-(1-methyl-1,2,4-triazol-3-yl)-3-pyridyl]amino]ethyl]-4,7-dimethyl-3-(3-piperidyl)pyrazolo[3,4-c]isoquinolin-5-one (8.00 mg, 0.02 mmol) in 0.5 mL DCM was added formaldehyde (6.10 μL, 2.26 mg, 0.08 mmol) and the resulting mixture was kept stirred at rt for 15 min. sodium triacetoxyborohydride (9.56 mg, 0.05 mmol) was then added to the reaction mixture and the resulting mixture was kept stirred at rt for 1 h. LCMS analysis indicated the reaction was complete. The crude was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product. LCMS calculated for C₂₈H₃₃ClN₉O (M+H)⁺ m/z=546.2. found: 546.2.

### Examples 278-281

Examples 278-281 in Table 40 were prepared similarly as described for Example 277.

TABLE 40

| | | | LCMS |
|---|---|---|---|
| Example | | | m/z |
| # | Chemical name | Structure | (M + H)⁺ |
| 278 | 9-[1-[[6-chloro-2-(1-methyl- 1,2,4-triazol-3-yl)-3- pyridyl]amino]ethyl]-3-[1-(2- hydroxyethyl)-3-piperidyl]- 4,7-dimethyl-pyrazolo[3,4- c]isoquinolin-5-one | 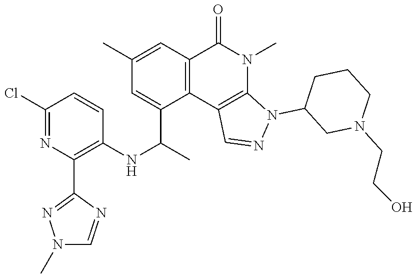 [SMILES: Cc1cc(C(C)Nc2ccc(Cl)nc2-c2ncn(C)n2)c2c(c1)c(=O)n(C)c1c2cnn1C1CCCN(CCO)C1] | 576.3 |
| 279 | 9-[1-[[6-chloro-2-(1- methylpyrazol-4-yl)-3- pyridyl]amino]ethyl]-4,7- dimethyl-3-(1-methyl-3- piperidyl)pyrazolo[3,4- c]isoquinolin-5-one | 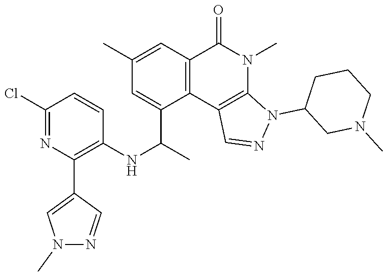 [SMILES: Cc1cc(C(C)Nc2ccc(Cl)nc2-c2cnn(C)c2)c2c(c1)c(=O)n(C)c1c2cnn1C1CCCN(C)C1] | 545.2 |
| 280 | 9-[1-[[6-chloro-2-(1- methylpyrazol-4-yl)-3- pyridyl]amino]ethyl]-3-[1-(2- hydroxyethyl)-3-piperidyl]- 4,7-dimethyl-pyrazolo[3,4- c]isoquinolin-5-one | 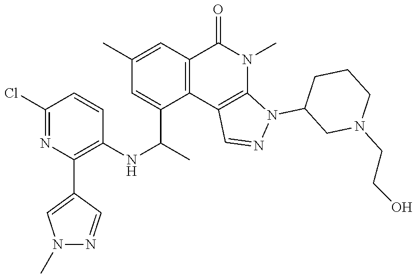 [SMILES: Cc1cc(C(C)Nc2ccc(Cl)nc2-c2cnn(C)c2)c2c(c1)c(=O)n(C)c1c2cnn1C1CCCN(CCO)C1] | 575.2 |
| 281 | methyl 3-[9-[1-[[6-chloro-2-(1- methylpyrazol-4-yl)-3- pyridyl]amino]ethyl]-4,7- dimethyl-5-oxo-pyrazolo[3,4- c]isoquinolin-3-yl]piperidine- 1-carboxylate | 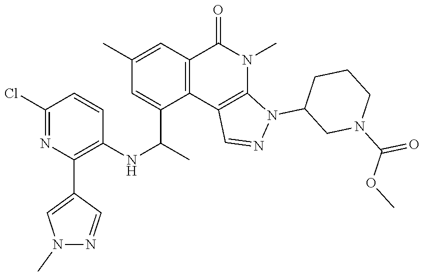 [SMILES: COC(=O)N1CCCC(n2ncc3c4c(C(C)Nc5ccc(Cl)nc5-c5cnn(C)c5)cc(C)cc4c(=O)n(C)c32)C1] | 589.2 |

### Example 282. 5-[6-Chloro-3-[1-[4,7-dimethyl-3-(1-methyl-4-piperidyl)-5-oxo-pyrazolo[3,4-c]isoquinolin-9-yl]ethoxy]-2-pyridyl]-N-methyl-pyridine-2-carboxamide

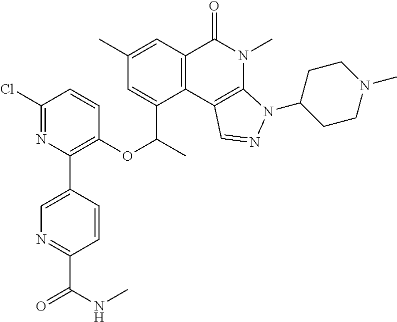 [SMILES: CNC(=O)c1ccc(-c2nc(Cl)ccc2OC(C)c2cc(C)cc3c(=O)n(C)c4c(cnn4C4CCN(C)CC4)c23)cn1]

### Step 1: benzyl 4-[9-[1-[(2-bromo-6-chloro-3-pyridyl)oxy]ethyl]-4,7-dimethyl-5-oxo-pyrazolo[3,4-c]isoquinolin-3-yl]piperidine-1-carboxylate

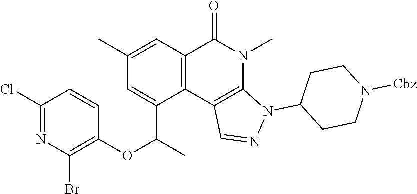 [SMILES: Cc1cc(C(C)Oc2ccc(Cl)nc2Br)c2c(c1)c(=O)n(C)c1c2cnn1C1CCN(C(=O)OCc2ccccc2)CC1]

To a stirred solution of 2-bromo-6-chloro-pyridin-3-ol (38.78 mg, 0.19 mmol) in 0.2 mL DMF was added sodium hydride (6.70 mg, 0.17 mmol) and the resulting mixture was kept stirred at room temperature (rt) for 1 h. Benzyl 4-[9-(1-bromoethyl)-4,7-dimethyl-5-oxo-pyrazolo[3,4-c]isoquinolin-3-yl]piperidine-1-carboxylate (Example 158, step 1) (50.0 mg, 0.09 mmol) was then added to the reaction mixture and the resulting mixture was kept stirred at 65° C. for 12 h. LCMS analysis indicated the reaction was complete. The reaction was quenched with water and extracted with 2×2 mL ethyl acetate (EA). The combined organics were concentrated to dryness and the residue was purified by silica gel column chromatography, eluted with dichloromethane (DCM) and MeOH to provide the desired product (42.00 mg, 67.9%) as a white solid. LCMS calculated for C₃₂H₃₂BrClN₅O₄ (M+H)⁺ m/z=664.1/666.1; found 664.1/666.1.

### Step 2: 5-[6-chloro-3-[1-[4,7-dimethyl-5-oxo-3-(4-piperidyl)pyrazolo[3,4-c]isoquinolin-9-yl]ethoxy]-2-pyridyl]-N-methyl-pyridine-2-carboxamide

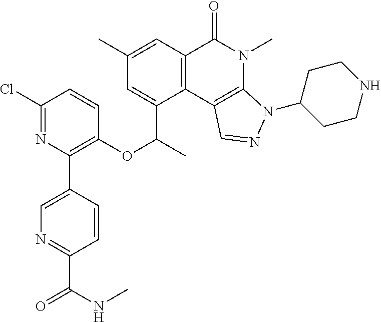 [SMILES: CNC(=O)c1ccc(-c2nc(Cl)ccc2OC(C)c2cc(C)cc3c(=O)n(C)c4c(cnn4C4CCNCC4)c23)cn1]

A mixture of benzyl 4-[9-[1-[(2-bromo-6-chloro-3-pyridyl)oxy]ethyl]-4,7-dimethyl-5-oxo-pyrazolo[3,4-c]isoquinolin-3-yl]piperidine-1-carboxylate (40.0 mg, 0.06 mmol), tetrakis(triphenylphosphine)palladium(0) (13.9 mg, 0.01 mmol), tripotassium phosphate (38.3 mg, 0.18 mmol) and N-methyl-5-(4,4,5,5-tetramethyl-1,3,2-dioxaborolan-2-yl)pyridine-2-carboxamide (18.92 mg, 0.07 mmol) in 1.5 mL dioxane/H₂O(5:1) was bubbled with N₂ for 1 min. The reaction mixture was stirred at 80° C. for 12 h under nitrogen atmosphere. LCMS analysis indicated the reaction was complete. The reaction was quenched with water and extracted with EA. The crude was concentrated and 1 mL HBr in acetic acid (33 wt. %) was added at 0° C. The resulting mixture kept stirred at 0° C. for 15 min. The reaction mixture was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product (22 mg, 62.4%) as a white solid. LCMS calculated for C₃₁H₃₃ClN₇O₃ (M+H)⁺ m/z=586.2; found 586.2.

### Step 3: 5-[6-chloro-3-[1-[4,7-dimethyl-3-(1-methyl-4-piperidyl)-5-oxo-pyrazolo[3,4-c]isoquinolin-9-yl]ethoxy]-2-pyridyl]-N-methyl-pyridine-2-carboxamide

To a stirred solution of 5-[6-chloro-3-[1-[4,7-dimethyl-5-oxo-3-(4-piperidyl)pyrazolo[3,4-c]isoquinolin-9-yl]ethoxy]-2-pyridyl]-N-methyl-pyridine-2-carboxamide (7.00 mg, 0.01 mmol) in 0.5 mL DCM was added formaldehyde (4.85 μL, 1.79 mg, 0.06 mmol) and the resulting mixture was kept stirred at rt for 15 min. sodium triacetoxyborohydride (7.59 mg, 0.04 mmol) was then added to the reaction mixture and the resulting mixture was kept stirred at rt for 1 h. LCMS analysis indicated the reaction was complete. The crude was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product (4.60 mg, 64.2%). LCMS calculated for C₃₂H₃₅ClN₇O₃ (M+H)⁺ m/z=600.2. found: 600.2.

### Example 283: 5-[3-[1-[3-(1-Acetyl-4-piperidyl)-4,7-dimethyl-5-oxo-pyrazolo[3,4-c]isoquinolin-9-yl]ethoxy]-6-chloro-2-pyridyl]-N-methyl-pyridine-2-carboxamide

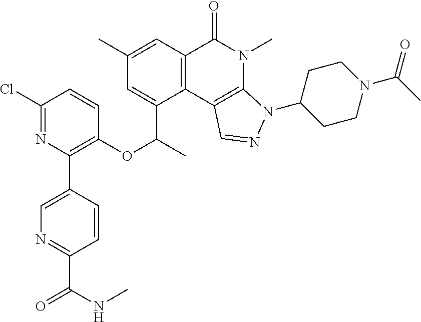 [SMILES: CNC(=O)c1ccc(-c2nc(Cl)ccc2OC(C)c2cc(C)cc3c(=O)n(C)c4c(cnn4C4CCN(C(C)=O)CC4)c23)cn1]

To a stirred solution of 5-[6-chloro-3-[1-[4,7-dimethyl-5-oxo-3-(4-piperidyl)pyrazolo[3,4-c]isoquinolin-9-yl]ethoxy]-2-pyridyl]-N-methyl-pyridine-2-carboxamide (7.00 mg, 0.01 mmol) in 0.5 mL DCM was added N-ethyl-N-isopropyl-propan-2-amine (6.62 uL, 4.63 mg, 0.04 mmol) and acetyl chloride (1.28 uL, 1.41 mg, 0.02 mmol) and the resulting mixture was kept stirred at rt for 10 min. LCMS analysis indicated the reaction was complete. The crude was diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product. LCMS calculated for C₃₃H₃₅ClN₇O₄ (M+H)⁺ m/z=628.2; found 628.2.

### Example 284: 10-(1-((6-Chloro-2-(1-methyl-1H-1,2,4-triazol-3-yl)pyridin-3-yl)amino)ethyl)-3-(hydroxymethyl)-8-methyl-4,5-dihydro-3H,6H-2,2a,5a-triazaaceanthrylen-6-one

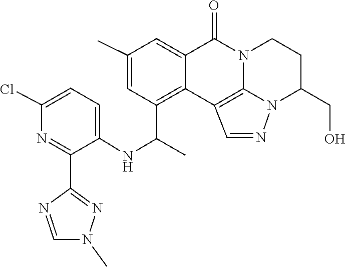 [SMILES: Cc1cc(C(C)Nc2ccc(Cl)nc2-c2ncn(C)n2)c2c(c1)c(=O)n1c3c2cnn3C(CO)CC1]

### Step 1: 9-bromo-4-(3-((tert-butyldimethylsilyl)oxy) propyl)-3-(4-methoxybenzyl)-7-methyl-3,4-dihydro-5H-pyrazolo[3,4-c]isoquinolin-5-one

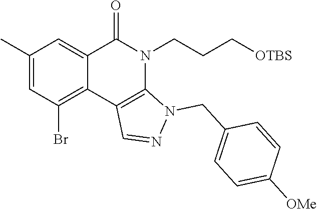 [SMILES: COc1ccc(Cn2ncc3c4c(Br)cc(C)cc4c(=O)n(CCCO[Si](C)(C)C(C)(C)C)c32)cc1]

The titled compound was prepared using similar procedures as described from Step 1 to Step 3 of Example 26 with (3-bromopropoxy) (tert-butyl)dimethylsilane replacing iodomethane in Step 2. The resulting mixture was diluted with water and extracted with EtOAc. The combined organics were washed with sat. NaCl, dried with anhydrous sodium sulfate, filtered, and concentrated under reduced pressure. The residue was purified by silica gel column chromatography, eluted with DCM and ethyl acetate to provide the desired product as a white solid. LCMS calculated for C₂₈H₃₇BrN₃O₃Si (M+H)⁺ m/z=570.2; found 570.2.

### Step 2: 9-bromo-4-(3-hydroxypropyl)-3-(4-methoxybenzyl)-7-methyl-3,4-dihydro-5H-pyrazolo[3,4-c]isoquinolin-5-one

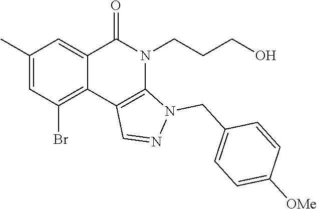 [SMILES: COc1ccc(Cn2ncc3c4c(Br)cc(C)cc4c(=O)n(CCCO)c32)cc1]

9-Bromo-4-(3-((tert-butyldimethylsilyl)oxy) propyl)-3-(4-methoxybenzyl)-7-methyl-3,4-dihydro-5H-pyrazolo[3,4-c]isoquinolin-5-one (3.7 g, 6.5 mmol) was dissolved in 60 mL of 4N HCl in dioxane. After stirring for 30 min at r.t., the mixture was diluted with water and extracted with EtOAc. The combined organics were dried with anhydrous sodium sulfate, filtered, and concentrated under reduced pressure. The residue was purified by silica gel column chromatography, eluted with hexanes and ethyl acetate to provide the desired product as a white solid (2.8 g, 94% yield). LCMS calculated for C₂₂H₂₃BrN₃O₃ (M+H)⁺ m/z=456.1; found 456.1.

### Step 3: 3-(9-bromo-3-(4-methoxybenzyl)-7-methyl-5-oxo-3,5-dihydro-4H-pyrazolo[3,4-c]isoquinolin-4-yl) propanal

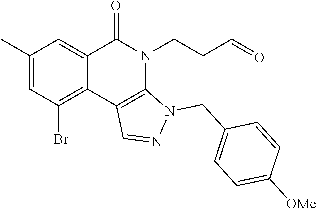 [SMILES: COc1ccc(Cn2ncc3c4c(Br)cc(C)cc4c(=O)n(CCC=O)c32)cc1]

To a solution of 9-bromo-4-(3-hydroxypropyl)-3-(4-methoxybenzyl)-7-methyl-3,4-dihydro-5H-pyrazolo[3,4-c]isoquinolin-5-one (2.8 g, 6.14 mmol) in 100 mL of DCM was added Dess-Martin periodinane (3.9 g, 9.2 mmol) at 0° C. After stirring at r.t. for overnight, the mixture was diluted with water and extracted with EtOAc. The combined organics were washed with NaHSO₃ and brine, then dried with anhydrous sodium sulfate, filtered, and concentrated under reduced pressure. The residue was purified by silica gel column chromatography, eluted with hexanes and ethyl acetate to provide the desired product as a white solid (1.5 g, 54% yield). LCMS calculated for C₂₂H₂₁BrN₃O₃ (M+H)⁺ m/z=454.1; found 454.1.

### Step 4: 9-bromo-4-(3-hydroxypent-4-en-1-yl)-3-(4-methoxybenzyl)-7-methyl-3,4-dihydro-5H-pyrazolo[3,4-c]isoquinolin-5-one

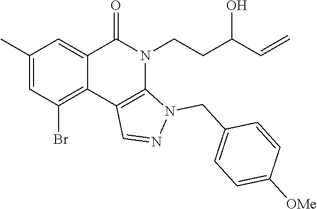 [SMILES: C=CC(O)CCn1c(=O)c2cc(C)cc(Br)c2c2cnn(Cc3ccc(OC)cc3)c21]

To a solution of 3-(9-bromo-3-(4-methoxybenzyl)-7-methyl-5-oxo-3,5-dihydro-4H-pyrazolo[3,4-c]isoquinolin-4-yl) propanal (1.0 g, 2.2 mmol) in 20 mL of THF was slowly added vinylmagnesium bromide solution (3.3 mL, 1M in THF, 3.3 mmol) at −15° C. After stirring at −15° C. for 1.5 h, the mixture was quenched with sat. NH₄Cl and extracted with EtOAc. The combined organics were dried with anhydrous sodium sulfate, filtered, and concentrated under reduced pressure. The residue was purified by silica gel column chromatography, eluted with hexanes and ethyl acetate to provide the desired product as a white solid (1.0 g, 94% yield). LCMS calculated for C₂₄H₂₅BrN₃O₃ (M+H)⁺ m/z=482.1; found 482.1.

### Step 5: 5-(9-bromo-7-methyl-5-oxo-3,5-dihydro-4H-pyrazolo[3,4-c]isoquinolin-4-yl)pent-1-en-3-yl ethyl carbonate

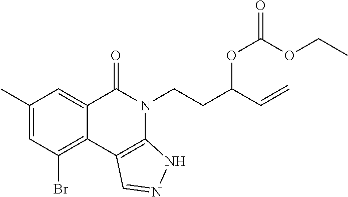 [SMILES: C=CC(CCn1c(=O)c2cc(C)cc(Br)c2c2cn[nH]c21)OC(=O)OCC]

To a solution of 9-bromo-4-(3-hydroxypent-4-en-1-yl)-3-(4-methoxybenzyl)-7-methyl-3,4-dihydro-5H-pyrazolo[3,4-c]isoquinolin-5-one (1.0 g, 2.07 mmol) in 20 mL of DCM was added pyridine (0.5 mL, 6.22 mmol) and ethyl carbonochloridate (0.4 mL, 4.15 mmol) at 0° C. After stirring at r.t. for 2 h, the mixture was diluted with water and extracted with DCM. The combined organics were dried with anhydrous sodium sulfate, filtered, and concentrated under reduced pressure. The residue was dissolved in 10 mL of 50% TFA in DCM at 0° C. After stirring at 60° C. for 30 min, the mixture was quenched with sat. NaHCO₃ and extracted with DCM. The combined organics were dried with anhydrous sodium sulfate, filtered, and concentrated under reduced pressure. The residue was purified by silica gel column chromatography, eluted with dichloromethane and ethyl acetate to provide the desired product as a white solid (645 mg, 71% yield). LCMS calculated for C₁₉H₂₁BrN₃O₄ (M+H)⁺ m/z=434.1; found 434.1.

### Step 6: 10-bromo-8-methyl-3-vinyl-4,5-dihydro-3H,6H-2,2a,5a-triazaaceanthrylen-6-one

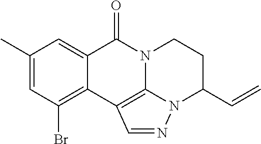 [SMILES: C=CC1CCn2c(=O)c3cc(C)cc(Br)c3c3cnn1c32]

To a solution of 5-(9-bromo-7-methyl-5-oxo-3,5-dihydro-4H-pyrazolo[3,4-c]isoquinolin-4-yl)pent-1-en-3-yl ethyl carbonate (209 mg, 0.48 mmol) in 2 mL of THF was added tris(dibenzylideneacetone)dipalladium (88.1 mg, 0.1 mmol) and 1,4-bis(diphenylphosphino) butane (82.1 mg, 0.19 mmol) at r.t. and degassed with nitrogen three times. After stirring at 60° C. for 30 min, the mixture was diluted with water and extracted with EtOAc. The combined organics were dried with anhydrous sodium sulfate, filtered, and concentrated under reduced pressure. The residue was purified by silica gel column chromatography, eluted with hexane and ethyl acetate to provide the desired product as a white solid (124 mg, 75% yield). LCMS calculated for C₁₆H₁₅BrN₃O (M+H)⁺ m/z=344.0; found 344.0.

### Step 7: 10-acetyl-8-methyl-3-vinyl-4,5-dihydro-3H,6H-2,2a,5a-triazaaceanthrylen-6-one

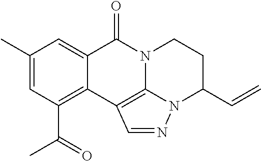 [SMILES: C=CC1CCn2c(=O)c3cc(C)cc(C(C)=O)c3c3cnn1c32]

A mixture of 10-bromo-8-methyl-3-vinyl-4,5-dihydro-3H,6H-2,2a,5a-triazaaceanthrylen-6-one (124 mg, 0.36 mmol), PdCl₂(PPh₃)₂ (51 mg, 0.07 mmol), and tributyl(1-ethoxyvinyl)stannane (260 mg, 0.72 mmol) in dioxane (2 mL) was heated at 100° C. for 2 h under nitrogen atmosphere. Upon cooling to room temperature, the mixture was added 2 N HCl and stirred for 30 min. Then 200 mg CsF was added and stirred for another 30 min before the mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a white solid (74 mg, 67%). LCMS calculated for C₁₈H₁₈N₃O₂ (M+H)⁺ m/z=308.1; found 308.1.

### Step 8: 10-(1-hydroxyethyl)-8-methyl-3-vinyl-4,5-dihydro-3H,6H-2,2a,5a-triazaaceanthrylen-6-one

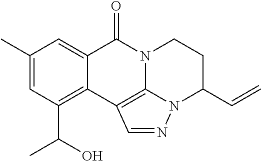 [SMILES: C=CC1CCn2c(=O)c3cc(C)cc(C(C)O)c3c3cnn1c32]

To a mixture of 10-acetyl-8-methyl-3-vinyl-4,5-dihydro-3H,6H-2,2a,5a-triazaaceanthrylen-6-one (74 mg, 0.24 mmol) in 2 mL of THF was added LAH (0.36 mL, 0.36 mmol, 1 M solution in THF) at −78° C. The resulting mixture was stirred at same temperature for 30 min, and then quenched with sat. NH₄Cl. The mixture was diluted with water and extracted with EtOAc. The combined organics were washed with sat. NaCl and dried with anhydrous sodium sulfate, filtered, and concentrated under reduced pressure. The residue was purified by silica gel column chromatography, eluted with DCM and ethyl acetate to provide the desired product as a white solid (55 mg, 74%). LCMS calculated for C₁₈H₂₀N₃O₂ (M+H)⁺ m/z=310.2; found 310.2.

### Step 9: 10-(1-bromoethyl)-8-methyl-3-vinyl-4,5-dihydro-3H,6H-2,2a,5a-triazaaceanthrylen-6-one

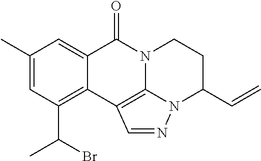 [SMILES: C=CC1CCn2c(=O)c3cc(C)cc(C(C)Br)c3c3cnn1c32]

To a mixture of 10-(1-hydroxyethyl)-8-methyl-3-vinyl-4,5-dihydro-3H,6H-2,2a,5a-triazaaceanthrylen-6-one (20 mg, 0.06 mmol) in DCM (1 mL) was added PBr₃ (35 mg, 0.13 mmol). The resulting mixture was stirred at rt for 2 h. The mixture was then diluted with water and extracted with DCM. The combined organics were washed with sat. NaCl, dried with anhydrous sodium sulfate, filtered, and concentrated under reduced pressure. The residue was used directly in the next step without further purification.

### Step 10: 10-(1-((6-chloro-2-(1-methyl-1H-1,2,4-triazol-3-yl)pyridin-3-yl)amino)ethyl)-8-methyl-3-vinyl-4,5-dihydro-3H,6H-2,2a,5a-triazaaceanthrylen-6-one

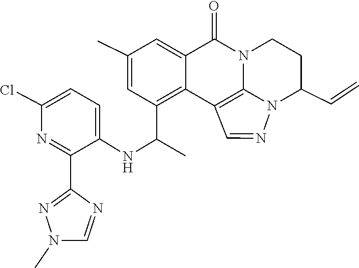 [SMILES: C=CC1CCn2c(=O)c3cc(C)cc(C(C)Nc4ccc(Cl)nc4-c4ncn(C)n4)c3c3cnn1c32]

To a solution of 10-(1-bromoethyl)-8-methyl-3-vinyl-4,5-dihydro-3H,6H-2,2a,5a-triazaaceanthrylen-6-one (23 mg, 0.066 mmol) in 0.6 mL of DMF was added 6-chloro-2-(1-methyl-1H-1,2,4-triazol-3-yl)pyridin-3-amine (26 mg, 0.12 mmol). After stirring at 60° C. for 1 h, the mixture was diluted with water and extracted with EtOAc. The combined organics were dried with anhydrous sodium sulfate, filtered, and concentrated under reduced pressure. The residue was purified by silica gel column chromatography, eluted with hexane and ethyl acetate to provide the desired product as a white solid (28 mg, 90% yield). LCMS calculated for C₂₆H₂₆ClN₈O (M+H)⁺ m/z=501.2; found 501.2.

### Step 11: 10-(1-((6-chloro-2-(1-methyl-1H-1,2,4-triazol-3-yl)pyridin-3-yl)amino)ethyl)-8-methyl-6-oxo-4,5-dihydro-3H,6H-2,2a,5a-triazaaceanthrylene-3-carbaldehyde

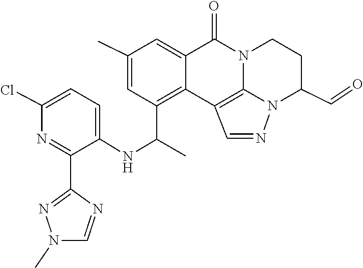 [SMILES: Cc1cc(C(C)Nc2ccc(Cl)nc2-c2ncn(C)n2)c2c(c1)c(=O)n1c3c2cnn3C(C=O)CC1]

To a solution of 10-(1-((6-chloro-2-(1-methyl-1H-1,2,4-triazol-3-yl)pyridin-3-yl)amino)ethyl)-8-methyl-3-vinyl-4,5-dihydro-3H,6H-2,2a,5a-triazaaceanthrylen-6-one (28 mg, 0.06 mmol) in 1 mL of THF and 0.5 mL of water was added sodium periodate (48 mg, 0.22 mmol) and Osmium tetroxide (0.07 mL, 0.01 mmol, 4% in water). After stirring at r.t. for 2 h, the mixture was diluted with water and extracted with EtOAc. The combined organics were dried with anhydrous sodium sulfate, filtered, and concentrated under reduced pressure. The residue was purified by silica gel column chromatography, eluted with hexane and ethyl acetate to provide the desired product as a white solid (12 mg, 43% yield). LCMS calculated for C₂₅H₂₄ClN₈O₂ (M+H)⁺ m/z=503.2; found 503.2.

### Step 12: 10-(1-((6-chloro-2-(1-methyl-1H-1,2,4-triazol-3-yl)pyridin-3-yl)amino)ethyl)-3-(hydroxymethyl)-8-methyl-4,5-dihydro-3H,6H-2,2a,5a-triazaaceanthrylen-6-one

To a solution of 10-(1-((6-chloro-2-(1-methyl-1H-1,2,4-triazol-3-yl)pyridin-3-yl)amino)ethyl)-8-methyl-6-oxo-4,5-dihydro-3H,6H-2,2a,5a-triazaaceanthrylene-3-carbaldehyde (6 mg, 0.01 mmol) in 0.5 mL of MeOH was added sodium borohydride (1 mg, 0.02 mml) at 0° C. After stirring at 0° C. for 30 min, the mixture was diluted with water and extracted with EtOAc. The combined organics were dried with anhydrous sodium sulfate, filtered, and concentrated under reduced pressure. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₂₆ClN₈O₂ (M+H)⁺ m/z=505.2; found 505.2.

### Examples 285-289

Examples 285-289 in Table 41 were prepared similarly as described for Example 284.

TABLE 41

| | | | LCMS |
|---|---|---|---|
| Example | | | m/z |
| # | Chemical name | Structure | (M + H)⁺ |
| 285 | 10-(1-((6-chloro-2-(2- methyl-2H-tetrazol-5- yl)pyridin-3-yl)amino)ethyl)- 3-(hydroxymethyl)-8- methyl-4,5-dihydro-3H,6H- 2,2a,5a-triazaaceanthrylen-6- one | 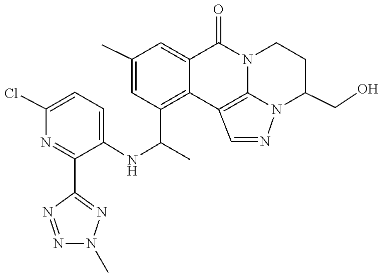 [SMILES: Cc1cc(C(C)Nc2ccc(Cl)nc2-c2nnn(C)n2)c2c(c1)c(=O)n1c3c2cnn3C(CO)CC1] | 506.2 |
| 286 | 10-(1-((6-chloro-2-(1,3,4- oxadiazol-2-yl)pyridin-3- yl)amino)ethyl)-3- (hydroxymethyl)-8-methyl- 4,5-dihydro-3H,6H-2,2a,5a- triazaaceanthrylen-6-one | 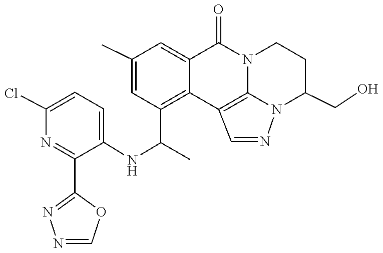 [SMILES: Cc1cc(C(C)Nc2ccc(Cl)nc2-c2nnco2)c2c(c1)c(=O)n1c3c2cnn3C(CO)CC1] | 492.2 |
| 287 | 10-(1-((6-chloro-2-(1- methyl-1H-pyrazol-4- yl)pyridin-3-yl)amino)ethyl)- 3-(hydroxymethyl)-8- methyl-4,5-dihydro-3H,6H- 2,2a,5a-triazaaceanthrylen-6- one | 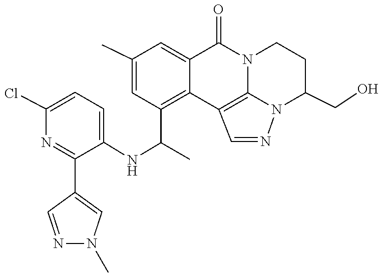 [SMILES: Cc1cc(C(C)Nc2ccc(Cl)nc2-c2cnn(C)c2)c2c(c1)c(=O)n1c3c2cnn3C(CO)CC1] | 504.2 |
| 288 | 10-(1-((6-chloro-2-(3- methyl-1H-pyrazol-4- yl)pyridin-3-yl)amino)ethyl)- 3-(hydroxymethyl)-8- methyl-4,5-dihydro-3H,6H- 2,2a,5a-triazaaceanthrylen-6- one | 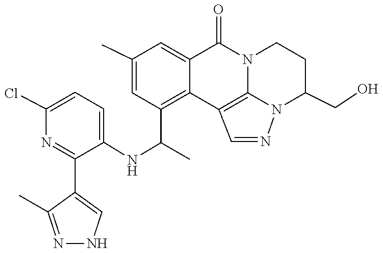 [SMILES: Cc1cc(C(C)Nc2ccc(Cl)nc2-c2c[nH]nc2C)c2c(c1)c(=O)n1c3c2cnn3C(CO)CC1] | 504.2 |
| 289 | 10-(1-((6-chloro-1′-methyl- 6′-oxo-1′,6′-dihydro-[2,3′- bipyridin]-3-yl)amino)ethyl)- 3-(hydroxymethyl)-8- methyl-4,5-dihydro-3H,6H- 2,2a,5a-triazaaceanthrylen-6- one | 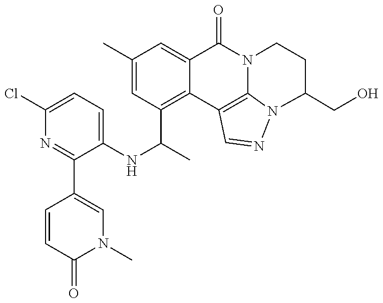 [SMILES: Cc1cc(C(C)Nc2ccc(Cl)nc2-c2ccc(=O)n(C)c2)c2c(c1)c(=O)n1c3c2cnn3C(CO)CC1] | 531.2 |

### Example 290: 10-(1-((6-Chloro-2-(1-methyl-1H-1,2,4-triazol-3-yl)pyridin-3-yl)amino)ethyl)-3-((dimethylamino)methyl)-8-methyl-4,5-dihydro-3H,6H-2,2a,5a-triazaaceanthrylen-6-one

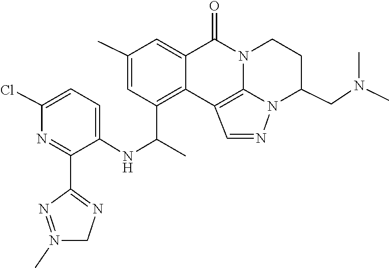

To a solution of 10-(1-((6-chloro-2-(1-methyl-1H-1,2,4-triazol-3-yl)pyridin-3-yl)amino)ethyl)-8-methyl-6-oxo-4,5-dihydro-3H,6H-2,2a,5a-triazaaceanthrylene-3-carbaldehyde (6 mg, 0.01 mmol) in 0.5 mL of ACN and 0.1 mL AcOH was added dimethylamine (0.12 mL, 0.12 mmol, 1 M in THF) and sodium triacetoxyborohydride (7.6 mg, 0.04 mml) at 0° C. After stirring at r.t. for 30 min, the mixture was diluted with water and extracted with EtOAc. The combined organics were dried with anhydrous sodium sulfate, filtered, and concentrated under reduced pressure. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₃₁ClN₉O (M+H)⁺ m/z=532.2; found 532.2.

### Examples 291-322

Examples 291-322 in Table 42 were prepared similarly as described for Example 36.

TABLE 42

| | | | LCMS |
|---|---|---|---|
| Example | | | m/z |
| # | Chemical name | Structure | (M + H)⁺ |
| 291 | 10-(1-((6-chloro-2-(4- hydroxypiperidin-1-yl)pyridin-3- yl)amino)ethyl)-8-methyl-4,5- dihydro-3H,6H-2,2a,5a- triazaaceanthrylen-6-one | 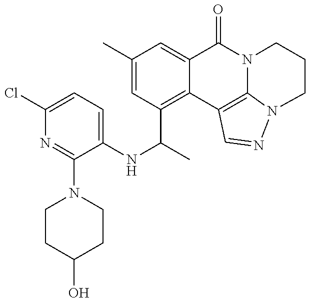 [SMILES: Cc1cc(C(C)Nc2ccc(Cl)nc2N2CCC(O)CC2)c2c(c1)c(=O)n1c3c2cnn3CCC1] | 493.2 |
| 292 | 1-(6-chloro-3-((1-(8-methyl-6-oxo- 4,5-dihydro-3H,6H-2,2a,5a- triazaaceanthrylen-10- yl)ethyl)amino)pyridin-2-yl)-N- methylpiperidine-4-carboxamide | 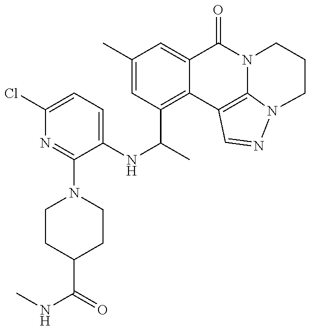 [SMILES: CNC(=O)C1CCN(c2nc(Cl)ccc2NC(C)c2cc(C)cc3c(=O)n4c5c(cnn5CCC4)c23)CC1] | 534.2 |
| 293 | 10-(1-((2-(4-acetylpiprerazin-1-yl)-6- chloropyridin-3-yl)amino)ethyl)-8- methyl-4,5-dihydro-3H,6H-2,2a,5a- triazaaceanthrylen-6-one | 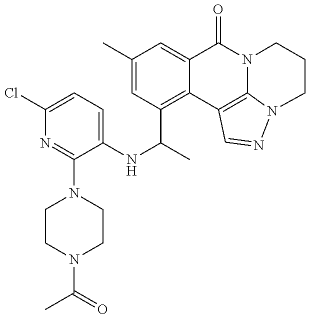 [SMILES: CC(=O)N1CCN(c2nc(Cl)ccc2NC(C)c2cc(C)cc3c(=O)n4c5c(cnn5CCC4)c23)CC1] | 520.2 |
| 294 | 10-(1-((6-chloro-2-(1,3,4-oxadiazol- 2-yl)pyridin-3-yl)amino)ethyl)-8- methyl-4,5-dihydro-3H,6H-2,2a,5a- triazaaceanthrylen-6-one | 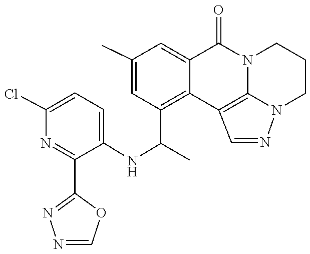 [SMILES: Cc1cc(C(C)Nc2ccc(Cl)nc2-c2nnco2)c2c(c1)c(=O)n1c3c2cnn3CCC1] | 462.1 |
| 295 | 3-(6-chloro-3-((1-(8-methyl-6-oxo- 4,5-dihydro-3H,6H-2,2a,5a- triazaaceanthrylen-10- yl)ethyl)amino)pyridin-2-yl)-1,2,4- oxadiazol-5(4H)-one | 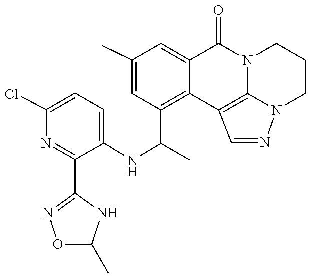 [SMILES: Cc1cc(C(C)Nc2ccc(Cl)nc2C2=NOC(C)N2)c2c(c1)c(=O)n1c3c2cnn3CCC1] | 478.1 |
| 296 | 10-(1-((6-chloro-2-(1-methyl-1H- 1,2,4-triazol-3-yl)pyridin-3- yl)amino)ethyl)-8-methyl-4,5- dihydro-3H,6H-2,2a,5a- triazaaceanthrylen-6-one | 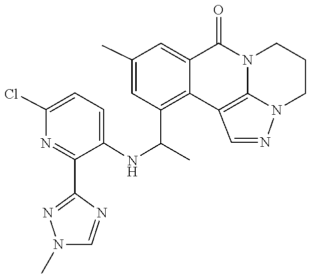 [SMILES: Cc1cc(C(C)Nc2ccc(Cl)nc2-c2ncn(C)n2)c2c(c1)c(=O)n1c3c2cnn3CCC1] | 475.2 |
| 297 | 10-(1-((6-chloro-2-(1-(2- hydroxyethyl)-1H-1,2,4-triazol-3- yl)pyridin-3-yl)amino)ethyl)-8- methyl-4,5-dihydro-3H,6H-2,2a,5a- triazaaceanthrylen-6-one | 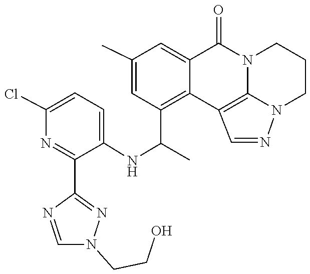 [SMILES: Cc1cc(C(C)Nc2ccc(Cl)nc2-c2ncn(CCO)n2)c2c(c1)c(=O)n1c3c2cnn3CCC1] | 505.2 |
| 298 | 10-(1-((6-chloro-2-(2-methyl-2H- tetrazol-5-yl)pyridin-3- yl)amino)ethyl)-8-methyl-4,5- dihydro-3H,6H-2,2a,5a- triazaaceanthrylen-6-one | 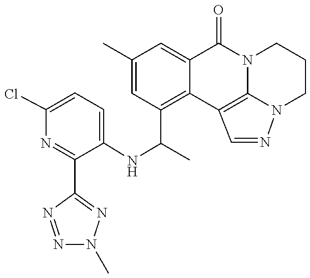 [SMILES: Cc1cc(C(C)Nc2ccc(Cl)nc2-c2nnn(C)n2)c2c(c1)c(=O)n1c3c2cnn3CCC1] | 476.2 |
| 299 | 10-(1-((6-chloro-2-(1-(tetrahydro- 2H-pyran-4-yl)-1H-pyrazol-4- yl)pyridin-3-yl)amino)ethyl)-8- methyl-4,5-dihydro-3H,6H-2,2a,5a- triazaaceanthrylen-6-one | 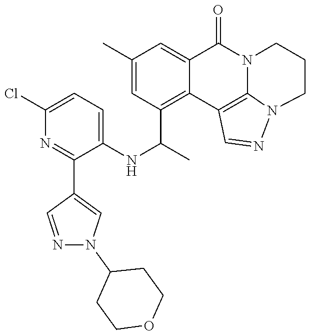 [SMILES: Cc1cc(C(C)Nc2ccc(Cl)nc2-c2cnn(C3CCOCC3)c2)c2c(c1)c(=O)n1c3c2cnn3CCC1] | 544.2 |
| 300 | 10-(1-((6-chloro-2-(1-(2- hydroxyethyl)-1H-pyrazol-4- yl)pyridin-3-yl)amino)ethyl)-8- methyl-4,5-dihydro-3H,6H-2,2a,5a- triazaaceanthrylen-6-one | 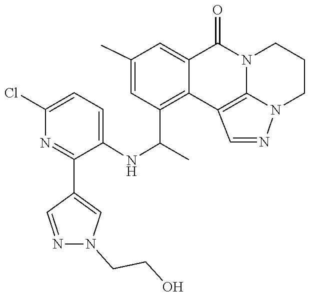 [SMILES: Cc1cc(C(C)Nc2ccc(Cl)nc2-c2cnn(CCO)c2)c2c(c1)c(=O)n1c3c2cnn3CCC1] | 504.2 |
| 301 | 10-(1-((6-chloro-2-(1-(1- methylpyridin-4-yl)-1H-pyrazol-4- yl)pyridin-3-yl)amino)ethyl)-8- methyl-4,5-dihydro-3H,6H-2,2a,5a- triazaaceanthrylen-6-one | 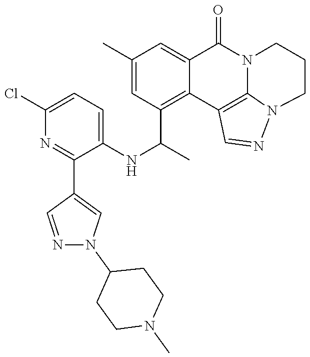 [SMILES: Cc1cc(C(C)Nc2ccc(Cl)nc2-c2cnn(C3CCN(C)CC3)c2)c2c(c1)c(=O)n1c3c2cnn3CCC1] | 557.3 |
| 302 | 10-(1-((6-chloro-2-(1-(1- methylpyridin-3-yl)-1H-pyrazol-4- yl)pyridin-3-yl)amino)ethyl)-8- methyl-4,5-dihydro-3H,6H-2,2a,5a- triazaaceanthrylen-6-one | 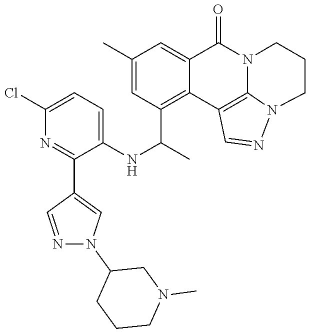 [SMILES: Cc1cc(C(C)Nc2ccc(Cl)nc2-c2cnn(C3CCCN(C)C3)c2)c2c(c1)c(=O)n1c3c2cnn3CCC1] | 557.3 |
| 303 | 10-(1-((6-chloro-2-(1-(1- methylpyrrolidin-3-yl)-1H-pyrazol- 4-yl)pyridin-3-yl)amino)ethyl)-8- methyl-4,5-dihydro-3H,6H-2,2a,5a- triazaaceanthrylen-6-one | 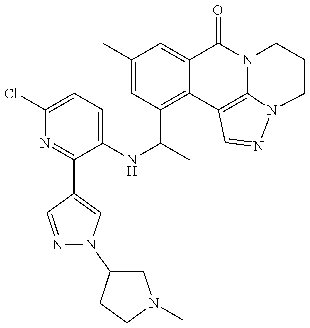 [SMILES: Cc1cc(C(C)Nc2ccc(Cl)nc2-c2cnn(C3CCN(C)C3)c2)c2c(c1)c(=O)n1c3c2cnn3CCC1] | 543.2 |
| 304 | 10-(1-((6-chloro-2-(1-(1- methylazetidin-3-yl)-1H-pyrazol-4- yl)pyridin-3-yl)amino)ethyl)-8- methyl-4,5-dihydro-3H,6H-2,2a,5a- triazaaceanthrylen-6-one | 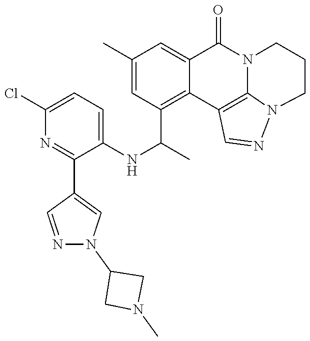 [SMILES: Cc1cc(C(C)Nc2ccc(Cl)nc2-c2cnn(C3CN(C)C3)c2)c2c(c1)c(=O)n1c3c2cnn3CCC1] | 529.2 |
| 305 | 4-(4-(6-chloro-3-((1-(8-methyl-6- oxo-4,5-dihydro-3H,6H-2,2a,5a- triazaaceanthrylen-10- yl)ethyl)amino)pyridin-2-yl)-1H- pyrazol-1-yl)-N,N- dimethylpiperidine-1-carboxamide | 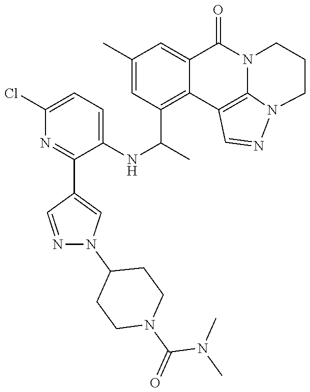 [SMILES: Cc1cc(C(C)Nc2ccc(Cl)nc2-c2cnn(C3CCN(C(=O)N(C)C)CC3)c2)c2c(c1)c(=O)n1c3c2cnn3CCC1] | 614.3 |
| 306 | 10-(1-((6-chloro-2′-methyl-[2,4′- bipyridin]-3-yl)amino)ethyl)-8- methyl-4,5-dihydro-3H,6H-2,2a,5a- triazaaceanthrylen-6-one | 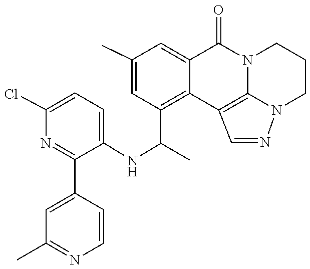 [SMILES: Cc1cc(C(C)Nc2ccc(Cl)nc2-c2ccnc(C)c2)c2c(c1)c(=O)n1c3c2cnn3CCC1] | 485.2 |
| 307 | 10-(1-((6-chloro-[2,3′-bipyridin]-3- yl)amino)ethyl)-8-methyl-4,5- dihydro-3H,6H-2,2a,5a- triazaaceanthrylen-6-one | 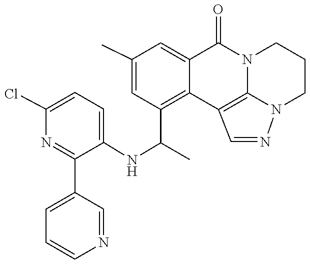 [SMILES: Cc1cc(C(C)Nc2ccc(Cl)nc2-c2cccnc2)c2c(c1)c(=O)n1c3c2cnn3CCC1] | 471.2 |
| 308 | 10-(1-((6-chloro-1′-methyl-6′-oxo- 1′,6′-dihydro-[2,3′-pyridin]-3- yl)amino)ethyl)-8-methyl-4,5- dihydro-3H,6H-2,2a,5a- triazaaceanthrylen-6-one | 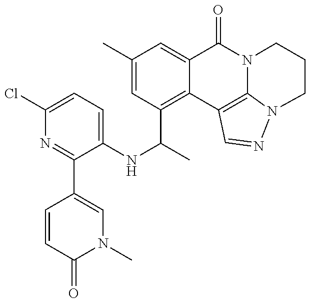 [SMILES: Cc1cc(C(C)Nc2ccc(Cl)nc2-c2ccc(=O)n(C)c2)c2c(c1)c(=O)n1c3c2cnn3CCC1] | 501.2 |
| 309 | 4-(6-chloro-3-((1-(8-methyl-6-oxo- 4,5-dihydro-3H,6H-2,2a,5a- triazaaceanthrylen-10- yl)ethyl)amino)pyridin-2-yl)-2- fluoro-N-methylbenzamide | 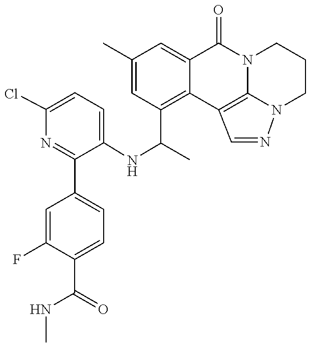 [SMILES: CNC(=O)c1ccc(-c2nc(Cl)ccc2NC(C)c2cc(C)cc3c(=O)n4c5c(cnn5CCC4)c23)cc1F] | 545.2 |
| 310 | 6-chloro-N-methyl-3-((1-(8-methyl- 6-oxo-4,5-dihydro-3H,6H-2,2a,5a- triazaaceanthrylen-10- yl)ethyl)amino)-[2,3′-bipyridine]-6′- carboxamide | 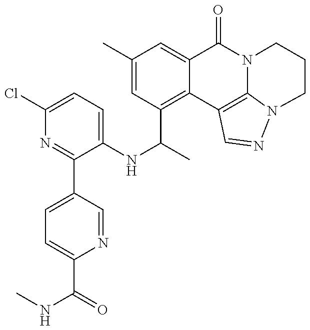 [SMILES: CNC(=O)c1ccc(-c2nc(Cl)ccc2NC(C)c2cc(C)cc3c(=O)n4c5c(cnn5CCC4)c23)cn1] | 528.2 |
| 311 | 10-(1-((6-chloro-6′-methoxy-[2,3′- bipyridin]-3-yl)amino)ethyl)-8- methyl-4,5-dihydro-3H,6H-2,2a,5a- triazaaceanthrylen-6-one | 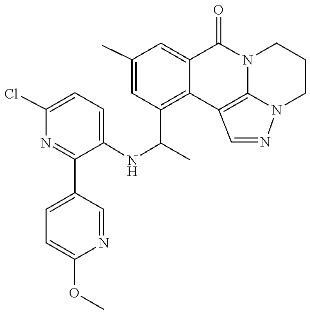 [SMILES: COc1ccc(-c2nc(Cl)ccc2NC(C)c2cc(C)cc3c(=O)n4c5c(cnn5CCC4)c23)cn1] | 501.2 |
| 312 | 10-(1-((6-chloro-2′-methoxy-[2,4′- bipyridin]-3-yl)amino)ethyl)-8- methyl-4,5-dihydro-3H,6H-2,2a,5a- triazaaceanthrylen-6-one | 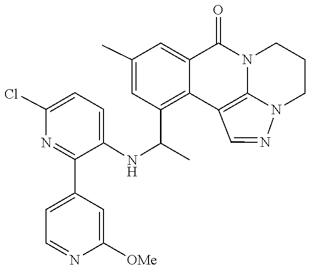 [SMILES: COc1cc(-c2nc(Cl)ccc2NC(C)c2cc(C)cc3c(=O)n4c5c(cnn5CCC4)c23)ccn1] | 501.2 |
| 313 | 10-(1-((6-chloro-2′-(dimethylamino)- [2,4′-bipyridin]-3-yl)amino)ethyl)-8- methyl-4,5-dihydro-3H,6H-2,2a,5a- triazaaceanthrylen-6-one | 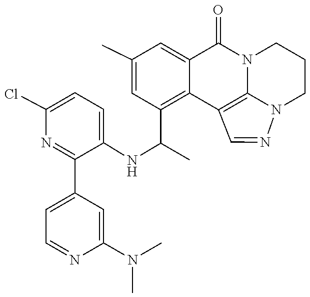 [SMILES: Cc1cc(C(C)Nc2ccc(Cl)nc2-c2ccnc(N(C)C)c2)c2c(c1)c(=O)n1c3c2cnn3CCC1] | 514.2 |
| 314 | 10-(1-((6-chloro-2-(2-methyl-1- oxoisoindolin-5-yl)pyridin-3- yl)amino)ethyl)-8-methyl-4,5- dihydro-3H,6H-2,2a,5a- triazaaceanthrylen-6-one | 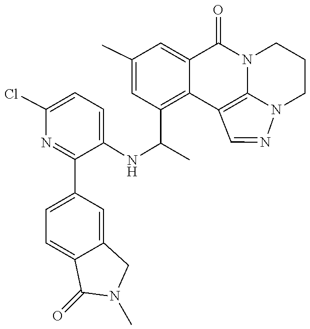 [SMILES: Cc1cc(C(C)Nc2ccc(Cl)nc2-c2ccc3c(c2)CN(C)C3=O)c2c(c1)c(=O)n1c3c2cnn3CCC1] | 539.2 |
| 315 | 10-(1-((6-chloro-2-(quinolin-6- yl)pyridin-3-yl)amino)ethyl)-8- methyl-4,5-dihydro-3H,6H-2,2a,5a- triazaaceanthrylen-6-one | 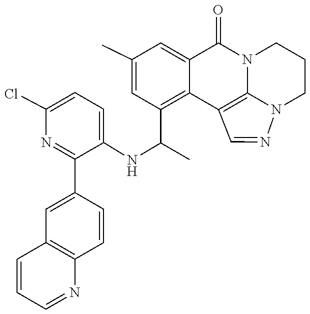 [SMILES: Cc1cc(C(C)Nc2ccc(Cl)nc2-c2ccc3ncccc3c2)c2c(c1)c(=O)n1c3c2cnn3CCC1] | 521.2 |
| 316 | 10-(1-((6-chloro-2-(1-methyl-1H- indazol-5-yl)pyridin-3- yl)amino)ethyl)-8-methyl-4,5- dihydro-3H,6H-2,2a,5a- triazaaceanthrylen-6-one | 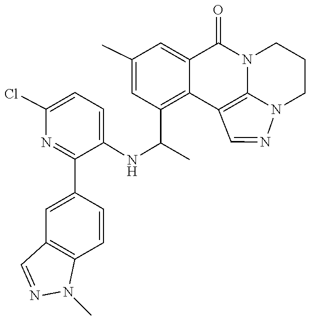 [SMILES: Cc1cc(C(C)Nc2ccc(Cl)nc2-c2ccc3c(cnn3C)c2)c2c(c1)c(=O)n1c3c2cnn3CCC1] | 524.2 |
| 317 | 2-fluoro-N-methyl-4-(6-methyl-3- ((1-(8-methyl-6-oxo-4,5-dihydro- 3H,6H-2,2a,5a-triazaaceanthrylen- 10-yl)ethyl)amino)pyridin-2- yl)benzamide | 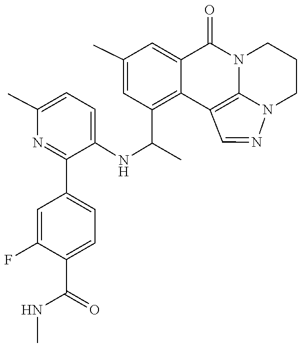 [SMILES: CNC(=O)c1ccc(-c2nc(C)ccc2NC(C)c2cc(C)cc3c(=O)n4c5c(cnn5CCC4)c23)cc1F] | 525.2 |
| 318 | 8-methyl-10-(1-((6-methyl-2-(1- (tetrahydro-2H-pyran-4-yl)-1H- pyrazol-4-yl)pyridin-3- yl)amino)ethyl)-4,5-dihydro-3H,6H- 2,2a,5a-triazaaceanthrylen-6-one | 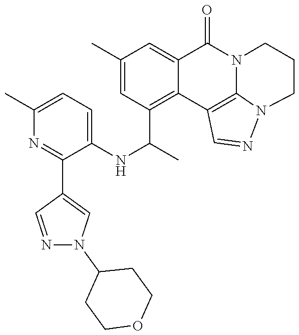 [SMILES: Cc1cc(C(C)Nc2ccc(C)nc2-c2cnn(C3CCOCC3)c2)c2c(c1)c(=O)n1c3c2cnn3CCC1] | 524.3 |
| 319 | 10-(1-((2-(1-(2-hydroxyethyl)-1H- pyrazol-4-yl)-6-methylpyridin-3- yl)amino)ethyl)-8-methyl-4,5- dihydro-3H,6H-2,2a,5a- triazaaceanthrylen-6-one | 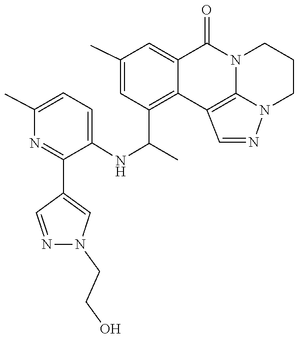 [SMILES: Cc1cc(C(C)Nc2ccc(C)nc2-c2cnn(CCO)c2)c2c(c1)c(=O)n1c3c2cnn3CCC1] | 484.2 |
| 320 | 10-(1-((2′,6-dimethyl-[2,4′- bipyridin]-3-yl)amino)ethyl)-8- methyl-4,5-dihydro-3H,6H-2,2a,5a- triazaaceanthrylen-6-one | 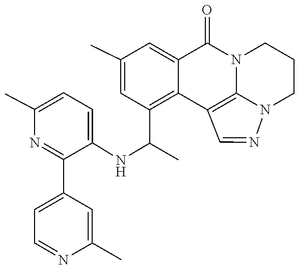 [SMILES: Cc1cc(C(C)Nc2ccc(C)nc2-c2ccnc(C)c2)c2c(c1)c(=O)n1c3c2cnn3CCC1] | 465.2 |
| 321 | 10-(1-((2-(1-cyclopropyl-1H- pyrazol-4-yl)-6-methylpyridin-3- yl)amino)ethyl)-8-methyl-4,5- dihydro-3H,6H-2,2a,5a- triazaaceanthrylen-6-one | 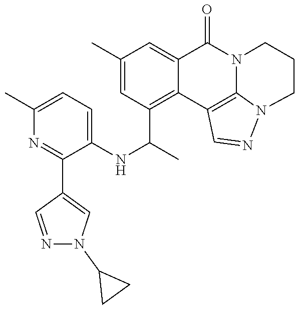 [SMILES: Cc1cc(C(C)Nc2ccc(C)nc2-c2cnn(C3CC3)c2)c2c(c1)c(=O)n1c3c2cnn3CCC1] | 480.3 |
| 322 | 7-(6-chloro-3-((1-(8-methyl-6-oxo- 4,5-dihydro-3H,6H-2,2a,5a- triazaaceanthrylen-10- yl)ethyl)amino)pyridin-2- yl)hexahydro-3H-oxazolo[3,4- a]pyrazin-3-one | 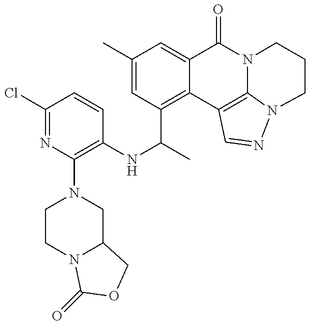 [SMILES: Cc1cc(C(C)Nc2ccc(Cl)nc2N2CCN3C(=O)OCC3C2)c2c(c1)c(=O)n1c3c2cnn3CCC1] | 534.2 |

### Example 323: 10-(1-((6-Chloro-2-(5-methyl-4,5,6,7-tetrahydropyrazolo[1,5-a]pyrazin-3-yl)pyridin-3-yl)amino)ethyl)-8-methyl-4,5-dihydro-3H,6H-2,2a,5a-triazaaceanthrylen-6-one

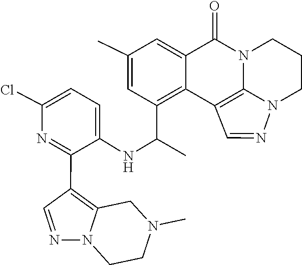 [SMILES: Cc1cc(C(C)Nc2ccc(Cl)nc2-c2cnn3c2CN(C)CC3)c2c(c1)c(=O)n1c3c2cnn3CCC1]

### Step 1: tert-butyl 3-(6-chloro-3-((1-(8-methyl-6-oxo-4,5-dihydro-3H, 6H-2,2a,5a-triazaaceanthrylen-10-yl)ethyl)amino)pyridin-2-yl)-6,7-dihydropyrazolo[1,5-a]pyrazine-5(4H)-carboxylate

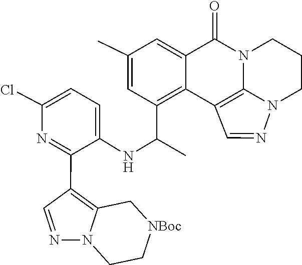 [SMILES: Cc1cc(C(C)Nc2ccc(Cl)nc2-c2cnn3c2CN(C(=O)OC(C)(C)C)CC3)c2c(c1)c(=O)n1c3c2cnn3CCC1]

To a mixture of tert-butyl 3-(3-amino-6-chloropyridin-2-yl)-6,7-dihydropyrazolo[1,5-a]pyrazine-5(4H)-carboxylate (intermediate 17) (15.2 mg, 0.04 mmol) and 10-(1-bromoethyl)-8-methyl-4,5-dihydro-3H,6H-2,2a,5a-triazaaceanthrylen-6-one (Example 36, step 6) (10.0 mg, 0.03 mmol) was added DMF (0.1 mL). The resulting mixture was heated at 60° C. for 1 hour. Mixture was cooled to room temperature, diluted with methanol, purified with by prep-HPLC (column: Sunfire prep 15 C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product. LCMS calculated for C₃₂H₃₆ClN₈O₃ (M+H)⁺ m/z=615.3; found: 615.3.

### Step 2: 10-(1-((6-chloro-2-(5-methyl-4,5,6,7-tetrahydropyrazolo[1,5-a]pyrazin-3-yl)pyridin-3-yl)amino)ethyl)-8-methyl-4,5-dihydro-3H,6H-2,2a,5a-triazaaceanthrylen-6-one

At room temperature, tert-butyl 3-(6-chloro-3-((1-(8-methyl-6-oxo-4,5-dihydro-3H,6H-2,2a,5a-triazaaceanthrylen-10-yl)ethyl)amino)pyridin-2-yl)-6,7-dihydropyrazolo[1,5-a]pyrazine-5(4H)-carboxylate (4.00 mg, 0.01 mmol) was treated with 1 to 1 mixture of trifluoroacetic acid and dichloromethane. The resulting mixture was stirred at room temperature for 30 minutes. Solvent was removed under reduced pressure. The crude product was added acetonitrile (0.5 mL), acetic acid (0.1 mL), formaldehyde (0.1 mL) and sodium triacetoxyhydroborate (0.82 mg, 3.88 μmol). the resulting mixture was stirred at rt for 2 hours. Upon completion of reaction, the residue was diluted with methanol and purified by prep-HPLC (column: Sunfire prep 15 C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₃₀ClN₈O (M+H)⁺ m/z=529.2; found: 529.2;

### Example 324: 10-(1-((6-Chloro-6′-((dimethylamino)methyl)-[2,3′-bipyridin]-3-yl)amino)ethyl)-8-methyl-4,5-dihydro-3H,6H-2,2a,5a-triazaaceanthrylen-6-one

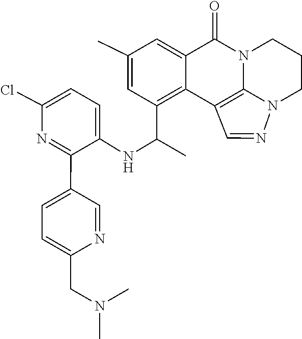 [SMILES: Cc1cc(C(C)Nc2ccc(Cl)nc2-c2ccc(CN(C)C)nc2)c2c(c1)c(=O)n1c3c2cnn3CCC1]

### Step 1: 10-(1-((2-bromo-6-chloropyridin-3-yl)amino)ethyl)-8-methyl-4,5-dihydro-3H,6H-2,2a,5a-triazaaceanthrylen-6-one

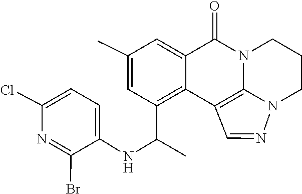 [SMILES: Cc1cc(C(C)Nc2ccc(Cl)nc2Br)c2c(c1)c(=O)n1c3c2cnn3CCC1]

The titled compound was prepared using similar procedures as described for Example 36 with 2-bromo-6-chloropyridin-3-amine replacing 2-aminobenzoic acid in Step 7. The resulting mixture was diluted with water and extracted with EtOAc. The combined organics were washed with sat. NaCl, dried with anhydrous sodium sulfate, filtered, and concentrated under reduced pressure. The residue was purified by silica gel column chromatography, eluted with DCM and ethyl acetate to provide the desired product as a white solid. LCMS calculated for C₂₁H₂₀BrClN₅O (M+H)⁺ m/z=472.1; found 472.1.

### Step 2: 6-chloro-3-((1-(8-methyl-6-oxo-4,5-dihydro-3H,6H-2,2a,5a-triazaaceanthrylen-10-yl)ethyl)amino)-[2,3′-bipyridine]-6′-carbaldehyde

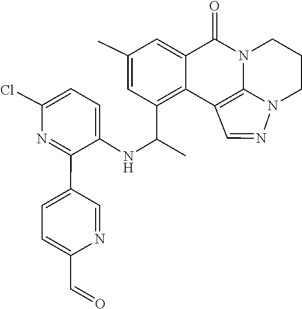 [SMILES: Cc1cc(C(C)Nc2ccc(Cl)nc2-c2ccc(C=O)nc2)c2c(c1)c(=O)n1c3c2cnn3CCC1]

To a solution of 10-(1-((2-bromo-6-chloropyridin-3-yl)amino)ethyl)-8-methyl-4,5-dihydro-3H,6H-2,2a,5a-triazaaceanthrylen-6-one (12 mg, 0.03 mmol) in 1.2 mL of toluene and 0.2 mL water was added tetrakis(triphenylphosphine)palladium (5.87 mg, 0.1 mmol), potassium carbonate (10.5 mg, 0.08 mmol) and 5-(4,4,5,5-tetramethyl-1,3,2-dioxaborolan-2-yl) picolinaldehyde (9 mg, 0.04 mmol) under nitrogen. After stirring at 80° C. for 1 h, the mixture was diluted with water and extracted with EtOAc. The combined organics were dried with anhydrous sodium sulfate, filtered, and concentrated under reduced pressure. The residue was purified by silica gel column chromatography, eluted with hexane and ethyl acetate to provide the desired product as a white solid (10 mg, 79% yield). LCMS calculated for C₂₇H₂₄ClN₆O₂ (M+H)⁺ m/z=499.2; found 499.2.

### Step 3: 10-(1-((6-chloro-6′-((dimethylamino)methyl)-[2,3′-bipyridin]-3-yl)amino)ethyl)-8-methyl-4,5-dihydro-3H,6H-2,2a,5a-triazaaceanthrylen-6-one

To a solution of 6-chloro-3-((1-(8-methyl-6-oxo-4,5-dihydro-3H,6H-2,2a,5a-triazaaceanthrylen-10-yl)ethyl)amino)-[2,3′-bipyridine]-6′-carbaldehyde (5 mg, 0.01 mmol) in 0.5 mL of ACN and 0.1 mL AcOH was added dimethylamine (0.06 mL, 0.12 mmol, 2 M in THF) and sodium triacetoxyborohydride (7.6 mg, 0.04 mml) at 0° C. After stirring at r.t. for 30 min, the mixture was diluted with water and extracted with EtOAc. The combined organics were dried with anhydrous sodium sulfate, filtered, and concentrated under reduced pressure. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₉H₃₁ClN₇O (M+H)⁺ m/z=528.2; found 528.2.

### Example 325: 3-(3-((1-(3,8-Dimethyl-6-oxo-4,5-dihydro-3H,6H-2,2a,5a-triazaaceanthrylen-10-yl)ethyl)amino)-6-methylpyridin-2-yl)-1,2,4-oxadiazol-5(4H)-one

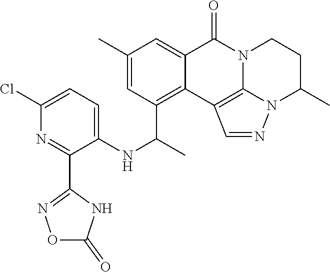 [SMILES: Cc1cc(C(C)Nc2ccc(Cl)nc2-c2noc(=O)[nH]2)c2c(c1)c(=O)n1c3c2cnn3C(C)CC1]

To a solution of 3-(6-chloro-3-((1-(3,8-dimethyl-6-oxo-4,5-dihydro-3H,6H-2,2a,5a-triazaaceanthrylen-10-yl)ethyl)amino)pyridin-2-yl)-1,2,4-oxadiazol-5(4H)-one (Example 70) (5 mg, 0.01 mmol) in 0.5 mL of dioxane and 0.1 mL water was added methylboronic acid (1.3 mg, 0.02 mmol), potassium phosphate tribasic (6.5 mg, 0.03 mmol) and P(t-Bu)3 Pd G2 (1 mg, 0.002 mmol) under nitrogen. After stirring at 80° C. for 1 h, the mixture was diluted with water and extracted with EtOAc. The combined organics were dried with anhydrous sodium sulfate, filtered, and concentrated under reduced pressure. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₂₆N₇O₃ (M+H)⁺ m/z=472.2; found 472.2.

### Example 326: 8-Methyl-10-(1-((6-methyl-2-(2-methyl-2H-tetrazol-5-yl)pyridin-3-yl)amino)ethyl)-4,5-dihydro-3H,6H-2,2a,5a-triazaaceanthrylen-6-one

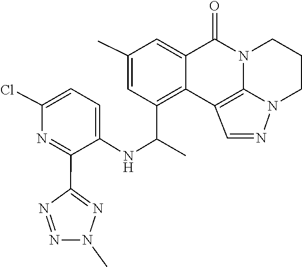 [SMILES: Cc1cc(C(C)Nc2ccc(Cl)nc2-c2nnn(C)n2)c2c(c1)c(=O)n1c3c2cnn3CCC1]

The titled compound was prepared using similar procedures as described for Example 325 with 10-(1-((6-chloro-2-(2-methyl-2H-tetrazol-5-yl)pyridin-3-yl)amino)ethyl)-8-methyl-4,5-dihydro-3H,6H-2,2a,5a-triazaaceanthrylen-6-one replacing 3-(6-chloro-3-((1-(3,8-dimethyl-6-oxo-4,5-dihydro-3H,6H-2,2a,5a-triazaaceanthrylen-10-yl)ethyl)amino)pyridin-2-yl)-1,2,4-oxadiazol-5(4H)-one. The resulting mixture was diluted with water and extracted with EtOAc. The combined organics were dried with anhydrous sodium sulfate, filtered, and concentrated under reduced pressure. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₆N₉O₃ (M+H)⁺ m/z=456.2; found 456.2.

### Example 327: 3-(3-((1-(3-Ethyl-4,7-dimethyl-5-oxo-4,5-dihydro-3H-pyrazolo[3,4-c]isoquinolin-9-yl)ethyl)amino)-6-methylpyridin-2-yl)-1,2,4-oxadiazol-5(4H)-one

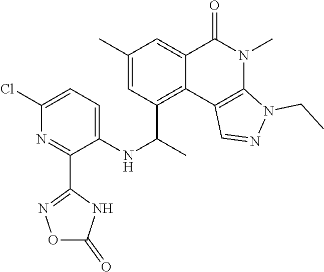 [SMILES: CCn1ncc2c3c(C(C)Nc4ccc(Cl)nc4-c4noc(=O)[nH]4)cc(C)cc3c(=O)n(C)c21]

The titled compound was prepared using similar procedures as described for Example 325 with 3-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one (Example 59) replacing 3-(6-chloro-3-((1-(3,8-dimethyl-6-oxo-4,5-dihydro-3H,6H-2,2a,5a-triazaaceanthrylen-10-yl)ethyl)amino)pyridin-2-yl)-1,2,4-oxadiazol-5(4H)-one. The resulting mixture was diluted with water and extracted with EtOAc. The combined organics were dried with anhydrous sodium sulfate, filtered, and concentrated under reduced pressure. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₄H₂₆N₇O₃ (M+H)⁺ m/z=460.2; found 460.2.

### Example 328: 10-(1-((6-Chloro-2-(1-methyl-1H-1,2,4-triazol-3-yl)pyridin-3-yl)amino)-2-hydroxyethyl)-8-methyl-4,5-dihydro-3H,6H-2,2a,5a-triazaaceanthrylen-6-one

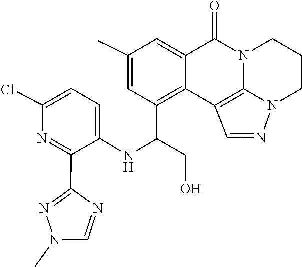 [SMILES: Cc1cc(C(CO)Nc2ccc(Cl)nc2-c2ncn(C)n2)c2c(c1)c(=O)n1c3c2cnn3CCC1]

### Step 1: 8-methyl-10-vinyl-4,5-dihydro-3H,6H-2,2a,5a-triazaaceanthrylen-6-one

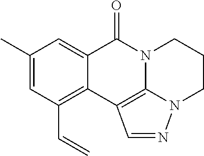 [SMILES: C=Cc1cc(C)cc2c(=O)n3c4c(cnn4CCC3)c12]

To the compound of 10-bromo-8-methyl-4,5-dihydro-3H,6H-2,2a,5a-triazaaceanthrylen-6-one (Example 36, step 3) (3.7 g, 11.63 mmol) was added Pd(dppf)Cl₂·DCM (0.85 g, 1.16 mmol), potassium vinyltrifluoroborate (4.67 g, 34.89 mmol) and potassium carbonate (4.82 g, 34.89 mmol) and 5 to 1 mixture of tetrahydrofuran and water (100 mL) at room temperature. The resulting mixture was heated at 70° C. for 2 hours. Upon completion of reaction based on LCMS analysis, mixture was filtered through Celite, filtrate was concentrated under reduced pressure. The residue was purified with silica gel chromatography, eluting from 0% to 100% ethyl acetate/hexanes to give desired product as a brown solid (2.50 g, 81%). LCMS calculated for C₁₆H₁₆N₃O (M+H)⁺ m/z=266.1; found: 266.1;

### Step 2: 8-methyl-10-(oxiran-2-yl)-4,5-dihydro-3H,6H-2,2a, 5a-triazaaceanthrylen-6-one

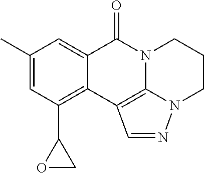 [SMILES: Cc1cc(C2CO2)c2c(c1)c(=O)n1c3c2cnn3CCC1]

To the solution of 8-methyl-10-vinyl-4,5-dihydro-3H,6H-2,2a,5a-triazaaceanthrylen-6-one (500 mg, 1.88 mmol) in dichloromethane (18 mL) was added m-chloroperoxybenzoic acid (975 mg, 5.65 mmol) at room temperature. The resulting mixture was stirred at room temperature for 3 hours. Upon completion of reaction based on LCMS analysis, the reaction was quenched with saturated water solution of sodium bicarbonate, organic layer was dried with sodium sulfate. Solvent was removed under reduced pressure to give desired crude product as orange color solid which was used in the next step directly. LCMS calculated for C₁₆H₁₆N₃O₂ (M+H)⁺ m/z=282.1; found: 282.1.

### Step 3: 10-(1-((6-chloro-2-(1-methyl-1H-1,2,4-triazol-3-yl)pyridin-3-yl)amino)-2-hydroxyethyl)-8-methyl-4,5-dihydro-3H,6H-2,2a,5a-triazaaceanthrylen-6-one

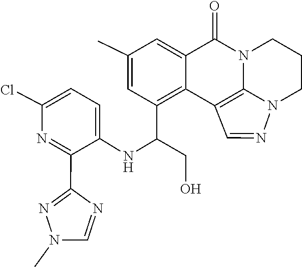 [SMILES: Cc1cc(C(CO)Nc2ccc(Cl)nc2-c2ncn(C)n2)c2c(c1)c(=O)n1c3c2cnn3CCC1]

To the solution of 8-methyl-10-(oxiran-2-yl)-4,5-dihydro-3H,6H-2,2a,5a-triazaaceanthrylen-6-one (10.0 mg, 0.04 mmol) in dichloromethane was added scandium (III) triflate (3.5 mg, 0.01 mmol) followed by addition of 6-chloro-2-(1-methyl-1H-1,2,4-triazol-3-yl)pyridin-3-amine (14.9 mg, 0.07 mmol) at room temperature. The resulting mixture was heated at 60° C. for 2 hours. Upon completion of reaction, the residue was diluted with methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a yellow solid. LCMS calculated for C₂₄H₂₄ClN₈O₂ (M+H)⁺ m/z=491.2; found: 491.2.

### Examples 329-333

Examples 329-333 in Table 43 were prepared similarly as described for Example 328.

TABLE 43

| | | | LCMS |
|---|---|---|---|
| Example | | | m/z |
| # | Chemical name | Structure | (M + H)⁺ |
| 329 | 10-(1-((6-chloro-2-(1-methyl-1H- pyrazol-4-yl)pyridin-3-yl)amino)-2- hydroxyethyl)-8-methyl-4,5-dihydro- 3H,6H-2,2a,5a-triazaaceanthrylen-6-one | 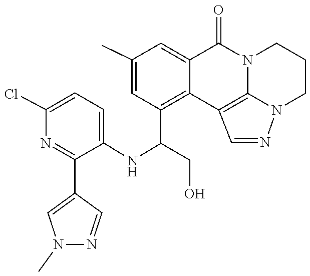 [SMILES: Cc1cc(C(CO)Nc2ccc(Cl)nc2-c2cnn(C)c2)c2c(c1)c(=O)n1c3c2cnn3CCC1] | 490.2 |
| 330 | 10-(1-((6-chloro-2-(1,5-dimethyl-1H- pyrazol-4-yl)pyridin-3-yl)amino)-2- hydroxyethyl)-8-methyl-4,5-dihydro- 3H,6H-2,2a,5a-triazaaceanthrylen-6-one | 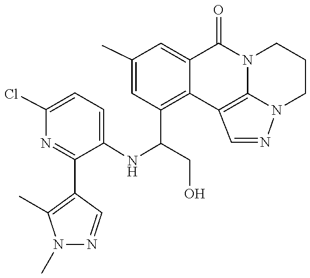 [SMILES: Cc1cc(C(CO)Nc2ccc(Cl)nc2-c2cnn(C)c2C)c2c(c1)c(=O)n1c3c2cnn3CCC1] | 504.2 |
| 331 | 10-(1-((6-chloro-2-(1,3,4-oxadiazol-2- yl)pyridin-3-yl)amino)-2-hydroxyethyl)- 8-methyl-4,5-dihydro-3H,6H-2,2a,5a- triazaaceanthrylen-6-one | 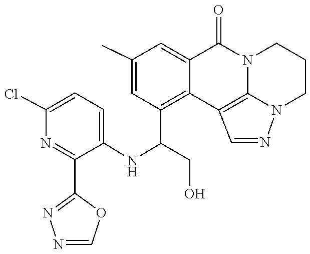 [SMILES: Cc1cc(C(CO)Nc2ccc(Cl)nc2-c2nnco2)c2c(c1)c(=O)n1c3c2cnn3CCC1] | 478.1 |
| 332 | 10-(1-((6-chloro-2-(2-methyl-2H- tetrazol-5-yl)pyridin-3-yl)amino)-2- hydroxyethyl)-8-methyl-4,5-dihydro- 3H,6H-2,2a5a-triazaaceanthrylen-6-one | 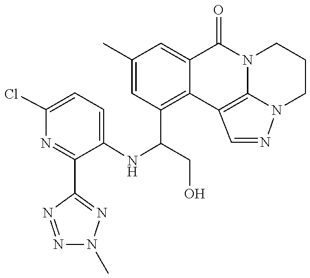 [SMILES: Cc1cc(C(CO)Nc2ccc(Cl)nc2-c2nnn(C)n2)c2c(c1)c(=O)n1c3c2cnn3CCC1] | 492.2 |
| 333 | 10-(1-((6-chloro-2-(2-methyl-1- oxoisoindolin-5-yl)pyridin-3-yl)amino)- 2-hydroxyethyl)-8-methyl-4,5-dihydro- 3H,6H-2,2a,5a-triazaaceanthrylen-6-one | 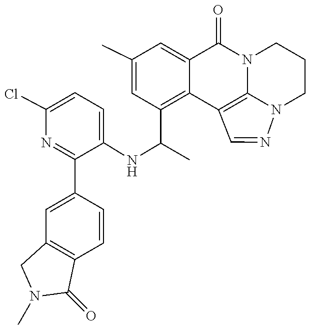 [SMILES: Cc1cc(C(C)Nc2ccc(Cl)nc2-c2ccc3c(c2)CN(C)C3=O)c2c(c1)c(=O)n1c3c2cnn3CCC1] | 555.2 |

### Example 334: 9-(1-((6-Chloro-2-(1-methyl-1H-pyrazol-4-yl)pyridin-3-yl)amino)-2-(4-methylpiperazin-1-yl)ethyl)-3-ethyl-4,7-dimethyl-3,4-dihydro-5H-pyrazolo[3,4-c]isoquinolin-5-one

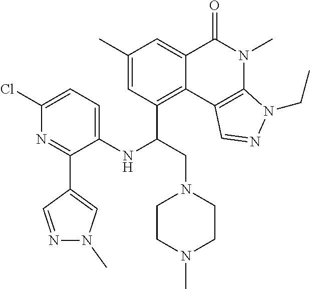 [SMILES: CCn1ncc2c3c(C(CN4CCN(C)CC4)Nc4ccc(Cl)nc4-c4cnn(C)c4)cc(C)cc3c(=O)n(C)c21]

### Step 1: 9-(1-((6-chloro-2-(1-methyl-1H-pyrazol-4-yl)pyridin-3-yl)amino)-2-hydroxyethyl)-3-ethyl-4,7-dimethyl-3,4-dihydro-5H-pyrazolo[3,4-c]isoquinolin-5-one

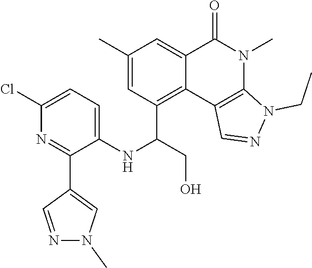 [SMILES: CCn1ncc2c3c(C(CO)Nc4ccc(Cl)nc4-c4cnn(C)c4)cc(C)cc3c(=O)n(C)c21]

The titled compound was prepared using similar procedures as described for Example 76 with 6-chloro-2-(1-methyl-1H-pyrazol-4-yl)pyridin-3-amine replacing 2-bromo-6-chloropyridin-3-amine in step 4. The crude product was purified by silica gel column chromatography, eluted with DCM and MeOH to provide the desired product as a brown solid. LCMS calculated for C₂₅H₂₇ClN₇O₂ (M+H)⁺ m/z=492.2; found 492.1.

### Step 2: 9-(2-((tert-butyldimethylsilyl)oxy)-1-((6-chloro-2-(1-methyl-1H-pyrazol-4-yl)pyridin-3-yl)amino)ethyl)-3-ethyl-4,7-dimethyl-3,4-dihydro-5H-pyrazolo[3,4-c]isoquinolin-5-one

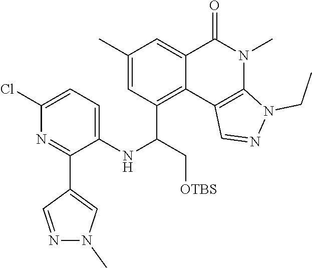 [SMILES: CCn1ncc2c3c(C(CO[Si](C)(C)C(C)(C)C)Nc4ccc(Cl)nc4-c4cnn(C)c4)cc(C)cc3c(=O)n(C)c21]

To a stirred solution of 9-(1-((6-chloro-2-(1-methyl-1H-pyrazol-4-yl)pyridin-3-yl)amino)-2-hydroxyethyl)-3-ethyl-4,7-dimethyl-3,4-dihydro-5H-pyrazolo[3,4-c]isoquinolin-5-one (120 mg, 0.24 mmol) in 2 mL DCM was added TBSCl (45 mg, 0.3 mmol), imidazole (20 mg, 0.3 mmol) at 0° C. The reaction mixture was stirred at rt for 12 h. LCMS analysis indicated the reaction was complete. The reaction was quenched with 30 mL water and extracted with 2×30 mL DCM. The combined organics were washed with saturated NaCl, dried with anhydrous sodium sulfate, filtered, and concentrated under reduced pressure. The residue was purified by silica gel column chromatography, eluted with 0˜10% EtOAc in DCM to provide the desired product (130 mg, 90%). LCMS calculated for C₃₁H₄₁ClN₇O₂Si (M+H)⁺ m/z=606.3; found 606.3.

### Step 3: 9-(1-((6-chloro-2-(1-methyl-1H-pyrazol-4-yl)pyridin-3-yl) (4-methoxybenzyl)amino)-2-hydroxyethyl)-3-ethyl-4,7-dimethyl-3,4-dihydro-5H-pyrazolo[3,4-c]isoquinolin-5-one

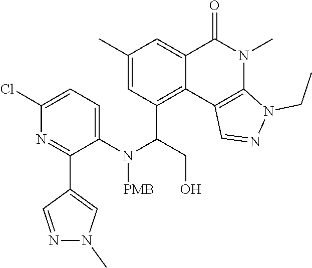

To a mixture of 9-(2-((tert-butyldimethylsilyl)oxy)-1-((6-chloro-2-(1-methyl-1H-pyrazol-4-yl)pyridin-3-yl)amino)ethyl)-3-ethyl-4,7-dimethyl-3,4-dihydro-5H-pyrazolo[3,4-c]isoquinolin-5-one (130 mg, 0.22 mmol) in DMF (2 mL) was added NaH (12 mg, 0.3 mmol) at 0° C. under nitrogen atmosphere. The resulting mixture was stirred at same temperature for 30 min before 4-methoxybenzyl bromide (60 mg, 0.3 mmol) was added. The reaction mixture was then slowly warmed to rt and stirred for 1 h. After completion, the reaction was carefully quenched with water at 0° C. while vigorously stirred. The mixture was then extracted with ethyl acetate (2×30 mL). The combined organics were dried with anhydrous sodium sulfate, filtered, and concentrated under reduced pressure. The residue was added 1 mL of THF and tetrabutylammonium fluoride (THF solution, 0.5 mL, 0.5 mmol) at 0° C. The reaction was stirred at 0° C. for 30 min before quenched with water and extracted with EtOAc. The combined organics were concentrated under reduced pressure. The residue was purified by silica gel column chromatography, eluted DCM/MeOH to provide the desired product (94 mg, 70%). LCMS calculated for C₃₃H₃₅ClN₇O₃ (M+H)⁺ m/z=612.2; found 612.3.

### Step 4: 2-((6-chloro-2-(1-methyl-1H-pyrazol-4-yl)pyridin-3-yl) (4-methoxybenzyl)amino)-2-(3-ethyl-4,7-dimethyl-5-oxo-4,5-dihydro-3H-pyrazolo[3,4-c]isoquinolin-9-yl) acetaldehyde

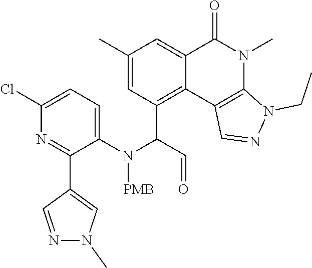

To a solution of 9-(1-((6-chloro-2-(1-methyl-1H-pyrazol-4-yl)pyridin-3-yl) (4-methoxybenzyl)amino)-2-hydroxyethyl)-3-ethyl-4,7-dimethyl-3,4-dihydro-5H-pyrazolo[3,4-c]isoquinolin-5-one (94 mg, 0.15 mmol) in 2 mL of DCM was added Dess-Martin periodinane (76 mg, 0.18 mmol) and NaHCO₃ (50 mg, 0.5 mmoL) at 0° C. After stirring at r.t. for overnight, the mixture was diluted with water and extracted with EtOAc. The organic layers were dried with anhydrous sodium sulfate, filtered, and concentrated under reduced pressure. The crude product was used directly in next step without further purification. LCMS calculated for C₃₃H₃₃ClN₇O₃ (M+H)⁺ m/z=610.2; found 610.2.

### Step 5: 9-(1-((6-chloro-2-(1-methyl-1H-pyrazol-4-yl)pyridin-3-yl)amino)-2-(4-methylpiperazin-1-yl)ethyl)-3-ethyl-4,7-dimethyl-3,4-dihydro-5H-pyrazolo[3,4-c]isoquinolin-5-one

To a solution of 2-((6-chloro-2-(1-methyl-1H-pyrazol-4-yl)pyridin-3-yl) (4-methoxybenzyl)amino)-2-(3-ethyl-4,7-dimethyl-5-oxo-4,5-dihydro-3H-pyrazolo[3,4-c]isoquinolin-9-yl) acetaldehyde (10.0 mg, 0.016 mmol) in 0.5 mL of DCM was added 1-methylpiperazine (10 uL) and sodium triacetoxyborohydride (4.2 mg, 0.02 mmol). After stirring at r.t. overnight, the mixture was quenched with water and extracted with EtOAc. The combined organics were washed with brine and dried with anhydrous sodium sulfate, filtered, and concentrated under reduced pressure. The residue was added 10% TfOH in TFA, the mixture was stirred at rt for 20 min before diluted with MeOH and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₃₀H₃₇ClN₉O (M+H)⁺ m/z=574.3; found: 574.2.

### Example A. pS473 AKT Assay Protocol

SKBR3 (PIK3CA WT), MCF7 (PIK3CA E545K), and T47D (PIK3CA H1047R) cells were cultured using 10-cm petri dishes with recommended medium plus 10% fetal bovine serum. One day before the assay, cells were seeded in 96-well plates at a final density of 10,000 cells per well. After overnight incubation in complete medium, cells were treated with different concentrations of PI3Kα inhibitors for 2 h. Cells were then fixed using 4% paraformaldehyde at room temperature for 20 min. Aspirate 4% paraformaldehyde, and wash cells using 1× regular phosphate buffered saline 3 times, 5 min each. Aspirate any residual phosphate buffered saline and block cells using 10% goat serum containing 1% bovine serum albumin and 0.3% Triton X-100 at room temperature for 1 h. Without any additional washing, primary antibodies (rabbit anti-pSer473 AKT) were diluted using blocking buffer and added at a final volume of 50 microliter per well. Keep assay plates with primary antibodies overnight at 4° C. Wash cells using 1× regular phosphate buffered saline 3 times, 5 min each. After the final wash, incubate cells with horseradish peroxidase-conjugated secondary antibodies (goat Anti-rabbit IgG) diluted using the same blocking buffer at room temperature for 1 h. Wash cells thoroughly using 1× regular phosphate buffered saline 3 times, 5 min each. Aspirate any residual phosphate buffered saline. Add Super-Signal ELISA Pico Chemiluminescent Substrate at a final volume of 100 microliter per well. Read plates on i3x Multi-Mode Microplate Reader and calculate IC₅₀ values using GraphPad Prism software.

Results of the assay described above are presented in Table A. Compounds denoted of the present disclosure showed IC₅₀ values in the following ranges:
A: IC₅₀≤1000 nM;
B: 1000 nM<IC₅₀≤5000 nM;
C: 5000 nM<IC₅₀≤10000 nM;
D: 10000 nM<IC₅₀≤30000 nM.
E: 30000 nM<IC₅₀

TABLE A

| | SKBR3_IC₅₀ | | |
|---|---|---|---|
| Compound | (nM) | MCF7_IC₅₀ (nM) | T47D_IC₅₀ (nM) |
| I-1 | E | E | B |
| I-2 | E | E | B |
| I-3 | E | E | B |

### Example B. pS473 AKT Assay Protocol with 2.5% FBS

T47D (PIK3CA H1047R) cells were cultured using 10-cm petri dishes with recommended medium plus 10% fetal bovine serum. One day before the assay, cells were seeded in 96-well plates. After overnight incubation, the medium was changed to DMEM with 2.5% fetal bovine serum (FBS) and the cells were treated with different concentrations of the compounds for 2 h. Cells were then fixed using 4% paraformaldehyde at room temperature for 20 min. Aspirate 4% paraformaldehyde, and wash cells using 1× regular phosphate buffered saline 3 times, 5 min each. Aspirate any residual phosphate buffered saline and block cells using 10% goat serum containing 1% bovine serum albumin and 0.3% Triton X-100 at room temperature for 1 h. Without any additional washing, primary antibodies (rabbit anti-pSer473 AKT) were diluted using blocking buffer and added at a final volume of 50 microliter per well. Keep assay plates with primary antibodies overnight at 4° C. Wash cells using 1× regular phosphate buffered saline 3 times, 5 min each. After the final wash, incubate cells with horseradish peroxidase-conjugated secondary antibodies (goat Anti-rabbit IgG) diluted using the same blocking buffer at room temperature for 1 h. Wash cells thoroughly using 1× regular phosphate buffered saline 3 times, 5 min each. Aspirate any residual phosphate buffered saline. Add Super-Signal ELISA Pico Chemiluminescent Substrate at a final volume of 100 microliter per well. Read plates on i3x Multi-Mode Microplate Reader and calculate IC₅₀ values using GraphPad Prism software.

Results of the assay described above are presented in Tables B—C. “+” indicates an IC₅₀ less than 1000 nM; “++” indicates an IC₅₀ greater than or equal to 1000 nM but less than 5000 nM; “+++” indicates an IC₅₀ greater than or equal to 5000 nM but less than 10000 nM; and “++++” indicates an IC₅₀ greater than or equal to 10000 nM.

TABLE B

| Compound | T47D_IC₅₀ (nM) | Compound | T47D_IC₅₀ (nM) |
|---|---|---|---|
| I-4 | + | I-5 | + |
| I-6 | + | I-7 | + |
| I-8 | + | I-9 | + |
| I-10 | + | I-11 | + |
| I-12 | + | I-13 | + |
| I-14 | + | I-15 | + |
| I-16 | + | I-17 | + |
| I-18 | + | I-19 | + |
| I-20 | + | I-21 | + |
| I-22 | + | I-23 | + |
| I-24 | + | I-25 | + |
| I-26 | + | I-27 | + |
| I-28 | + | I-29 | + |
| I-30 | + | I-31 | + |
| I-32 | + | I-33 | + |
| I-34 | + | I-35 | + |
| I-36 | + | I-37 | + |
| I-38 | + | I-39 | + |
| 40 | + | 41 | + |
| 42 | + | 43 | + |
| 44 | + | 45 | + |
| 46 | + | 47 | + |
| 48 | + | 49 | + |
| 50 | + | 51 | + |
| 52 | + | 53 | + |
| 54 | + | 55 | + |
| 56 | + | 57 | + |
| 58 | + | 59 | + |
| 60 | + | 61 | + |
| 62 | + | 63 | + |
| 64 | + | 65 | + |
| 66 | + | 67 | + |
| 68 | + | 69 | + |
| 70 | + | 71 | + |
| 72 | + | 73 | + |
| 74 | + | 75 | + |
| 76 | + | 77 | + |
| 78 | + | 79 | + |
| 80 | + | | |

TABLE C

| | T47D_IC₅₀ | | T47D_IC₅₀ | | T47D_IC₅₀ |
|---|---|---|---|---|---|
| Example | (nM) | Example | (nM) | Example | (nM) |
| 81 | + | 82 | + | 83 | + |
| 84 | + | 85 | + | 86 | + |
| 87 | + | 88 | + | 89 | + |
| 90 | + | 91 | + | 92 | + |
| 93 | + | 94 | + | 95 | + |
| 96 | + | 97 | + | 98 | + |
| 99 | + | 100 | + | 101 | + |
| 102 | ++ | 103 | + | 104 | + |
| 105 | + | 106 | + | 107 | + |
| 108 | + | 109 | + | 110 | + |
| 111 | + | 112 | + | 113 | + |
| 114 | + | 115 | + | 116 | + |
| 117 | + | 118 | + | 119 | + |
| 120 | + | 121 | + | 122 | + |
| 123 | + | 124 | + | 125 | + |
| 126 | + | 127 | + | 128 | + |
| 129 | + | 130 | + | 131 | + |
| 132 | + | 133 | + | 134 | + |
| 135 | + | 136 | + | 137 | + |
| 138 | + | 139 | + | 140 | + |
| 141 | + | 142 | + | 143 | + |
| 144 | + | 145 | + | 146 | + |
| 147 | + | 148 | + | 149 | + |
| 150 | + | 151 | + | 152 | + |
| 153 | + | 154 | + | 155 | + |
| 156 | + | 157 | + | 158 | + |
| 159 | + | 160 | + | 161 | + |
| 162 | + | 163 | + | 164 | + |
| 165 | + | 166 | + | 167 | + |
| 168 | + | 169 | + | 170 | + |
| 171 | + | 172 | + | 173 | + |
| 174 | + | 175 | + | 176 | + |
| 177 | + | 178 | + | 179 | + |
| 180 | + | 181 | + | 182 | + |
| 183 | + | 184 | + | 185 | + |
| 186 | + | 187 | + | 188 | + |
| 189 | + | 190 | + | 191 | + |
| 192 | + | 193 | + | 194 | + |
| 195 | + | 196 | + | 197 | + |
| 198 | + | 199 | + | 200 | + |
| 201 | + | 202 | + | 203 | + |
| 204 | + | 205 | + | 206 | + |
| 207 | + | 208 | + | 209 | + |
| 210 | + | 211 | + | 212 | + |
| 213 | + | 214 | + | 215 | + |
| 216 | + | 217 | + | 218 | + |
| 219 | + | 220 | + | 221 | + |
| 222 | + | 223 | + | 224 | + |
| 225 | + | 226 | + | 227 | + |
| 228 | + | 229 | + | 230 | + |
| 231 | + | 232 | + | 233 | + |
| 234 | + | 235 | + | 236 | + |
| 237 | + | 238 | + | 239 | + |
| 240 | + | 241 | + | 242 | + |
| 243 | + | 244 | + | 245 | + |
| 246 | + | 247 | + | 248 | + |
| 249 | + | 250 | + | 251 | + |
| 252 | + | 253 | + | 254 | + |
| 255 | + | 256 | + | 257 | + |
| 258 | + | 259 | + | 260 | + |
| 261 | + | 262 | + | 263 | + |
| 264 | + | 265 | + | 266 | + |
| 267 | + | 268 | + | 269 | + |
| 270 | + | 271 | + | 272 | + |
| 273 | + | 274 | + | 275 | + |
| 276 | + | 277 | + | 278 | + |
| 279 | + | 280 | + | 281 | + |
| 282 | + | 283 | + | 284 | + |
| 285 | + | 286 | + | 287 | + |
| 288 | + | 289 | + | 290 | + |
| 291 | + | 292 | + | 293 | + |
| 294 | + | 295 | + | 296 | + |
| 297 | + | 298 | + | 299 | + |
| 300 | + | 301 | + | 302 | + |
| 303 | + | 304 | + | 305 | + |
| 306 | + | 307 | + | 308 | + |
| 309 | + | 310 | + | 311 | + |
| 312 | + | 313 | + | 314 | + |
| 315 | + | 316 | + | 317 | + |
| 318 | + | 319 | + | 320 | + |
| 321 | + | 322 | + | 323 | + |
| 324 | + | 325 | + | 326 | + |
| 327 | + | 328 | + | 329 | + |
| 330 | + | 331 | + | 332 | + |
| 333 | + | 334 | + | | |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula I: 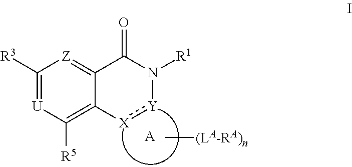
or a pharmaceutically acceptable salt thereof, wherein: [symbol]
is a single or double bond;
X is N or C;
Y is N or C;
Ring A is phenyl, 5- to 7-membered saturated or partially unsaturated monocyclic carbocyclyl, 5- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur;
each L^{A} is independently a covalent bond or optionally substituted bivalent C₁₋₆ aliphatic;
each R^{A} is independently oxo, halogen, —CN, —OR^{A1}, —SR^{A1}, —N(R^{A1})₂, —C(O)R^{A1}, —C(O)OR^{A1}, —C(O)N(R^{A1})₂, —C(O)NR^{A1}(OR^{A1}), —OC(O)R^{A1}—OC(O)N(R^{A1})₂, —OC(O)OR^{A1}, —OSO₂R^{A1}, —OSO₂N(R^{A1})₂, —N(R^{A1})C(O)R^{A1}, —NR^{A1}C(O)OR^{A1}, —NR^{A1}C(O)N(R^{A1})₂, —N(R^{A1})SO₂R^{A1}, —NR^{A1}S(O)₂N(R^{A1})₂, —NR^{A1}OR^{A1}, —NR^{A1}S(O)R^{A1}, —NR^{A1}S(O)N(R^{A1})₂, —S(O)R^{A1}, —SO₂R^{A1}, —S(O)N(R^{A1})₂, —SO₂N(R^{A1})₂, —SO₃R^{A1}, —C(═NR^{m})R^{A1}, —C(═NR^{m})N(R^{A1})₂, —NR^{A1}C(═NR^{m})R^{A1}, —NR^{A1}C(═NR^{m})N(R^{A1})₂, —NR^{A1}S(O)(═NR^{m})R^{A1}, —NR^{A1}S(O)(═NR^{m})N(R^{A1})₂, —OS(O)(═NR^{m})R^{A1}, —S(O)(═NR^{m})R^{A1}, —S(O)(═NR^{m})N(R^{A1})₂, —P(O)(R^{A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents;
or two L^{A}-R^{A} taken together with the atoms to which they are attached form a 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl is independently optionally substituted with 1, 2, 3, 4, 5, or 6 R^{AG} substituents;
R¹ is hydrogen or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur;
or R¹ and one of L^{A}-R^{A} taken together with the atoms to which they are attached form a 3- to 7-membered saturated or partially unsaturated monocyclic heterocyclyl having 1-3 heteroatoms independently selected from nitrogen, oxygen, and sulfur, or 5- to 14-membered saturated or partially unsaturated bicyclic heterocyclyl having 1-4 heteroatoms independently selected from nitrogen, oxygen, and sulfur, wherein each of the 3- to 7-membered monocyclic heterocyclyl and 5- to 14-membered bicyclic heterocyclyl is independently optionally substituted with 1, 2, 3, 4, 5, or 6 R^{AG} substituents;
Z is N or CR²;
U is N or CR⁴;
R² and R⁴ are each independently hydrogen, halogen, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur;
R³ is —F, —Cl, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur;
R⁵ is -L⁵-R^{5A};
L⁵ is a covalent bond or optionally substituted bivalent C₁₋₆ aliphatic;
or L⁵ and one of L^{A}-R^{A} taken together with the atoms to which they are attached form a 3- to 16-membered saturated or partially unsaturated monocyclic carbocyclyl, 5- to 16-membered saturated or partially unsaturated bicyclic carbocyclyl, 3- to 16-membered saturated or partially unsaturated monocyclic heterocyclyl having 1-3 heteroatoms independently selected from nitrogen, oxygen, and sulfur, or 5- to 16-membered saturated or partially unsaturated bicyclic heterocyclyl having 1-4 heteroatoms independently selected from nitrogen, oxygen, and sulfur, wherein each of the 3- to 16-membered monocyclic carbocyclyl, 5- to 16-membered bicyclic carbocyclyl, 3- to 16-membered monocyclic heterocyclyl, and 5- to 16-membered bicyclic heterocyclyl is independently optionally substituted with 1, 2, 3, 4, 5, or 6 R^{AG} substituents;
R^{5A} is —OR^{5A11}, —SR^{5A1}, —NHR^{5A12}, —N(R^{5A12})₂, —C(O)R^{5A1}, —C(O)N(R^{5A1})₂, —C(O)NR^{5A1} (OR^{5A1}), —OC(O)R^{5A1}, —OC(O)N(R^{5A1})₂, —OC(O)OR^{5A1}, —OSO₂R^{5A1}, —OSO₂N(R^{5A1})₂, —N(R^{5A1})C(O)R^{5A1}, —NR^{5A1}C(O)OR^{5A1}, —NR^{5A1}C(O)N(R^{5A1})₂, —N(R^{5A1})SO₂R^{5A1}, —NR^{5A1}S(O)₂N(R^{5A1})₂, —NR^{5A1}OR^{5A1}, —NR^{5A1}S(O)R^{5A1}, —NR^{5A1}S(O)N(R^{5A1})₂, —S(O)R^{5A1}, —SO₂R^{5A1}, —S(O)N(R^{5A1})₂, —SO₂N(R^{5A1})₂, —SO₃R^{5A1}, —C(═NR^{m})R^{5A1}, —C(═NR^{m})N(R^{5A1})₂, —NR^{5A1}C(═NR^{m})R^{5A1}, —NR^{5A1}C(═NR^{m})N(R^{5A1})₂, —NR^{5A1}S(O)(═NR^{m})R^{5A1}, —NR^{5A1}S(O)(═NR^{m})N(R^{5A1})₂, —OS(O)(═NR^{m})R^{5A1}, —S(O)(═NR^{m})R^{5A1}, —S(O)(═NR^{m})N(R^{5A1})₂, —P(O)(R^{5A1})₂, methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A} is independently optionally substituted with 1, 2, 3, 4, 5, or 6 R^{5AG} substituents; or
the methyl of R^{5A} is substituted with 1, 2, or 3 R^{5AG} substituents;
R^{A1} and R^{5A1} are each independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A1} or R^{5A1} is independently optionally substituted with 1, 2, 3, 4, 5, or 6 R^{G1} substituents;
or two R^{A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
or two R^{5A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
R^{5A11} is methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A11} is independently optionally substituted with 1, 2, 3, 4, 5, or 6 R^{G1} substituents; or
the methyl of R^{5A11} is substituted with 1, 2, or 3 R^{G1} substituents;
each R^{5A12} is independently C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A12} is independently optionally substituted with 1, 2, 3, 4, 5, or 6 R^{G1} substituents;
or two R^{5A12} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
R^{AG}, R^{5AG}, and R^{G1} are each independently halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
each R is independently hydrogen or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or
two R when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
each R^{m} is independently —OH, —CN, or R; and
n is 0, 1, 2, 3, or 4.

2. The compound of claim 1, or a pharmaceutically acceptable salt thereof, wherein: [symbol]
is a single or double bond;
X is N or C;
Y is N or C;
Ring A is phenyl, 5- to 7-membered saturated or partially unsaturated monocyclic carbocyclyl, 5- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur;
each L^{A} is independently a covalent bond or a bivalent C₁₋₆ aliphatic, wherein the bivalent C₁₋₆ aliphatic is optionally substituted with 1, 2, 3, or 4 independently selected R_{LA1} substituents;
each R^{A} is independently oxo, halogen, —CN, —OR^{A1}, —SR^{A1}, —N(R^{A1})₂, —C(O)R^{A1}, —C(O)OR^{A1}, —C(O)N(R^{A1})₂, —C(O)NR^{A1}(OR^{A1}), —OC(O)R^{A1}—OC(O)N(R^{A1})₂, —OC(O)OR^{A1}, —OSO₂R^{A1}, —OSO₂N(R^{A1})₂, —N(R^{A1})C(O)R^{A1}, —NR^{A1}C(O)OR^{A1}, —NR^{A1}C(O)N(R^{A1})₂, —N(R^{A1})SO₂R^{A1}, —NR^{A1}S(O)₂N(R^{A1})₂, —NR^{A1}OR^{A1}, —NR^{A1}S(O)R^{A1}, —NR^{A1}S(O)N(R^{A1})₂, —S(O)R^{A1}, —SO₂R^{A1}, —S(O)N(R^{A1})₂, —SO₂N(R^{A1})₂, —SO₃R^{A1}, —C(═NR^{m})R^{A1}, —C(═NR^{m})N(R^{A1})₂, —NR^{A1}C(═NR^{m})R^{A1}, —NR^{A1}C(═NR^{m})N(R^{A1})₂, —NR^{A1}S(O)(═NR^{m})R^{A1}, —NR^{A1}S(O)(═NR^{m})N(R^{A1})₂, —OS(O)(═NR^{m})R^{A1}, —S(O)(═NR^{m})R^{A1}, —S(O)(═NR^{m})N(R^{A1})₂, —P(O)(R^{A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents;
or two L^{A}-R^{A} taken together with the atoms to which they are attached form a 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl is independently optionally substituted with 1, 2, 3, 4, 5, or 6 R^{AG} substituents;
R¹ is hydrogen or a group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl are each optionally substituted with 1, 2, 3, or 4 independently selected R^{1A} substituents;
or R¹ and one of L^{A}-R^{A} taken together with the atoms to which they are attached form a 3- to 7-membered saturated or partially unsaturated monocyclic heterocyclyl having 1-3 heteroatoms independently selected from nitrogen, oxygen, and sulfur, or 5- to 14-membered saturated or partially unsaturated bicyclic heterocyclyl having 1-4 heteroatoms independently selected from nitrogen, oxygen, and sulfur, wherein each of the 3- to 7-membered monocyclic heterocyclyl and 5- to 14-membered bicyclic heterocyclyl is independently optionally substituted with 1, 2, 3, 4, 5, or 6 R^{AG} substituents;
Z is N or CR²;
U is N or CR⁴;
R² and R⁴ are each independently hydrogen, halogen, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl are each optionally substituted with 1, 2, 3, or 4 independently selected R^{2A} substituents;
R³ is —F, —Cl, —CN, —OR, —SR, —N(R)₂, —C(O)R, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl are each optionally substituted with 1, 2, 3, or 4 independently selected R^{3A} substituents;
R⁵ is -L⁵-R^{5A};
L⁵ is a covalent bond or a bivalent C₁₋₆ aliphatic, wherein the bivalent C₁₋₆ aliphatic is optionally substituted with 1, 2, 3, or 4 independently selected R^{L5A} substituents;
or L⁵ and one of L^{A}-R^{A} taken together with the atoms to which they are attached form a 3- to 16-membered saturated or partially unsaturated monocyclic carbocyclyl, 5- to 16-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, or 5- to 16-membered saturated or partially unsaturated bicyclic heterocyclyl having 1-4 heteroatoms independently selected from nitrogen, oxygen, and sulfur, wherein each of the 3- to 16-membered monocyclic carbocyclyl, 5- to 16-membered bicyclic carbocyclyl, 3- to 16-membered monocyclic heterocyclyl, and 5- to 16-membered bicyclic heterocyclyl is independently optionally substituted with 1, 2, 3, 4, 5, or 6 R^{AG} substituents;
R^{5A} is —OR^{5A11}, —SR^{5A1}, —NHR^{5A12}, —N(R^{5A12})₂, —C(O)R^{5A1}, —C(O)N(R^{5A1})₂, —C(O)NR^{5A1} (OR^{5A1}), —OC(O)R^{5A1}, —OC(O)N(R^{5A1})₂, —OC(O)OR^{5A1}, —OSO₂R^{5A1}, —OSO₂N(R^{5A1})₂, —N(R^{5A1})C(O)R^{5A1}, —NR^{5A1}C(O)OR^{5A1}, —NR^{5A1}C(O)N(R^{5A1})₂, —N(R^{5A1})SO₂R^{5A1}, —NR^{5A1}S(O)₂N(R^{5A1})₂, —NR^{5A1}OR^{5A1}, —NR^{5A1}S(O)R^{5A1}, —NR^{5A1}S(O)N(R^{5A1})₂, —S(O)R^{5A1}, —SO₂R^{5A1}, —S(O)N(R^{5A1})₂, —SO₂N(R^{5A1})₂, —SO₃R^{5A1}, —C(═NR^{m})R^{5A1}, —C(═NR^{m})N(R^{5A1})₂, —NR^{5A1}C(═NR^{m})R^{5A1}, —NR^{5A1}C(═NR^{m})N(R^{5A1})₂, —NR^{5A1}S(O)(═NR^{m})R^{5A1}, —NR^{5A1}S(O)(═NR^{m})N(R^{5A1})₂, —OS(O)(═NR^{m})R^{5A1}, —S(O)(═NR^{m})R^{5A1}, —S(O)(═NR^{m})N(R^{5A1})₂, —P(O)(R^{5A1})₂, methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A} is independently optionally substituted with 1, 2, 3, 4, 5, or 6 R^{5AG} substituents; or
the methyl of R^{5A} is substituted with 1, 2, or 3 R^{5AG} substituents;
R^{A1} and R^{5A1} are each independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A1} or R^{5A1} is independently optionally substituted with 1, 2, 3, 4, 5, or 6 R^{G1} substituents;
or two R^{A1} when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{A11} substituents;
or two R^{5A1} when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{A11} substituents;
R^{5A11} is methyl, C₂₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₂₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A11} is independently optionally substituted with 1, 2, 3, 4, 5, or 6 R^{G1} substituents; or
the methyl of R^{5A11} is substituted with 1, 2, or 3 R^{G1} substituents;
each R^{5A12} is independently C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{5A12} is independently optionally substituted with 1, 2, 3, 4, 5, or 6 R^{G1} substituents;
or two R^{5A12} when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{5B12} substituents;
R^{AG}, R^{5AG}, and R^{G1} are each independently halogen, —CN, —OR, —SR, —N(R)₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl, 5- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{AG1} substituents;
each R is independently selected from hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl, 5- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{N} substituents; or
two R when attached to the same nitrogen atom are taken together to form a ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur, wherein the 3- to 7-membered saturated or partially unsaturated monocyclic heterocyclyl and 5- to 10-membered saturated or partially unsaturated bicyclic heterocyclyl are each optionally substituted with 1, 2, 3, or 4 independently selected R^{N} substituents;
each R^{LA1}, R^{1A}, R^{2A}, R^{3A}, R^{L5A}, R^{A11}, R^{5B12}, R^{AG1}, and R^{N} is independently selected from halogen, (CH₂)₀₋₄R^{o}, —(CH₂)₀₋₄OR^{o}, —O(CH₂)₀₋₄R^{o}, —O—(CH₂)₀₋₄C(O)OR^{o}, —(CH₂)₀₋₄CH(OR^{o})₂, —(CH₂)₀₋₄SR^{o}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{o})₂, —(CH₂)₀₋₄N(R^{o})C(O)R^{o}, —N(R^{o})C(S)R^{o}, —(CH₂)₀₋₄N(R^{o})C(O)NR^{o}₂, —N(R^{o})C(S)NR^{o}₂, —(CH₂)₀₋₄N(R^{o})C(O)OR^{o}, —N(R^{o})N(R^{o})C(O)R^{o}, —N(R^{o})N(R^{o})C(O)NR^{o}₂, —N(R^{o})N(R^{o})C(O)OR^{o}, —(CH₂)₀₋₄C(O)R^{o}, —C(S)R^{o}, —(CH₂)₀₋₄C(O)OR^{o}, —(CH₂)₀₋₄C(O)SR^{o}, —(CH₂)₀₋₄C(O)OSiR^{o}₃, —(CH₂)₀₋₄OC(O)R^{o}, —OC(O)(CH₂)₀₋₄SR^{o}, —(CH₂)₀₋₄SC(O)R^{o}, —(CH₂)₀₋₄C(O)NR^{o}₂, —C(S)NR^{o}₂, —C(S)SR^{o}, —SC(S)SR^{o}, —(CH₂)₀₋₄OC(O)NR^{o}₂, —C(O)N(OR^{o})R^{o}, —C(O)C(O)R^{o}, —C(O)CH₂C(O)R^{o}, —C(NOR^{o})R^{o}, —(CH₂)₀₋₄SSR^{o}, —(CH₂)₀₋₄S(O)₂R^{o}, —(CH₂)₀₋₄S(O)(═NR^{o})R^{o}, —(CH₂)₀₋₄S(O)₂OR^{o}, —(CH₂)₀₋₄OS(O)₂R^{o}, —(CH₂)₀₋₄—S(O)₂NR^{o}₂, —(CH₂)₀₋₄S(O)(═NR^{o})NR^{o}₂, —(CH₂)₀₋₄S(O)R^{o}, —N(R^{o})S(O)₂NR^{o}₂, —N(R^{o})S(O)₂R^{o}, —N(R^{o})S(O)(═NR^{o})R^{o}, —N(OR^{o})R^{o}, —C(NH)NR^{o}₂, —P(O)₂R^{o}, —P(O)R^{o}₂, —OP(O)R^{o}₂, —OP(O)(OR^{o})₂, —SiR^{o}₃, —(C₁₋₄ straight or branched alkylene)O—N(R^{o})₂, and —(C₁₋₄ straight or branched alkylene)C(O)O—N(R^{o})₂;
each R^{o} is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur;
or two independent occurrences of R^{o}, taken together with their intervening atoms, form a 3- to 12-membered saturated, partially unsaturated, or aryl mono- or bicyclic ring having 0-4 heteroatoms independently selected from nitrogen, oxygen, or sulfur;
each R^{m} is independently —OH, —CN, or R; and
n is 0, 1, 2, 3, or 4.

3. The compound of claim 1, or a pharmaceutically acceptable salt thereof, wherein the compound is of Formula II: 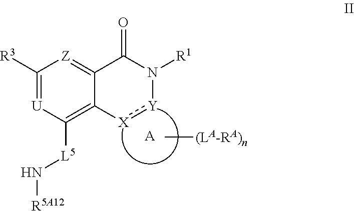
or a pharmaceutically acceptable salt thereof.

4. The compound of claim 1, or a pharmaceutically acceptable salt thereof, wherein the compound is of Formula III: 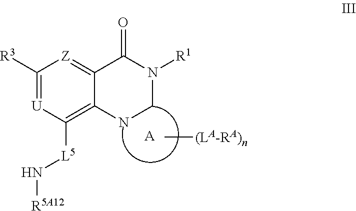
or a pharmaceutically acceptable salt thereof.

5. The compound of claim 1, or a pharmaceutically acceptable salt thereof, wherein X is N and Y is C.

6. The compound of claim 1, or a pharmaceutically acceptable salt thereof, wherein X is C and Y is C.

7. The compound of claim 1, or a pharmaceutically acceptable salt thereof, wherein Ring A is 5- to 6-membered monocyclic heteroaryl having 1-4 heteroatoms independently selected from nitrogen, oxygen, and sulfur.

8. The compound of claim 1, or a pharmaceutically acceptable salt thereof, wherein Ring A is selected from pyrazolyl, imidazolyl, triazolyl, and pyridyl.

9. The compound of claim 7, or a pharmaceutically acceptable salt thereof, wherein Ring A is 5-membered monocyclic heteroaryl having 1-2 nitrogen atoms.

10. The compound of claim 9, or a pharmaceutically acceptable salt thereof, wherein Ring A is pyrazolyl.

11. The compound of claim 1, or a pharmaceutically acceptable salt thereof, wherein L^{A} is a covalent bond or a bivalent C₁₋₂ aliphatic.

12. The compound of claim 1, or a pharmaceutically acceptable salt thereof, wherein L^{A} is a covalent bond.

13. The compound of claim 1, or a pharmaceutically acceptable salt thereof, wherein each R^{A} is independently C₁₋₆ alkyl, C₂₋₆ alkenyl, —OR^{A1}, —N(R^{A1})₂, —C(O)R^{A1}, —C(O)N(R^{A1})₂, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur, wherein the C₁₋₆ alkyl, C₂₋₆ alkenyl, 3- to 7-membered saturated or partially unsaturated monocyclic carbocyclyl, phenyl, 3- to 7-membered saturated or partially unsaturated monocyclic heterocyclyl, and 5- to 6-membered monocyclic heteroaryl of R^{A} are each independently optionally substituted with 1 or 2 R^{AG} substituents.

14. The compound of claim 1, or a pharmaceutically acceptable salt thereof, wherein each R^{A1} is independently C₁₋₆ alkyl or 3- to 7-membered saturated or partially unsaturated monocyclic heterocyclyl having 1-3 heteroatoms independently selected from nitrogen, oxygen, and sulfur, wherein each C₁₋₆ alkyl and 3- to 7-membered saturated or partially unsaturated monocyclic heterocyclyl of R^{A1} is optionally substituted with hydroxy or methoxy.

15. The compound of claim 1, or a pharmaceutically acceptable salt thereof, wherein each R^{A} is independently selected from phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, each of which is optionally substituted with 1, 2, 3, 4, 5, or 6 R^{AG} substituents.

16. The compound of claim 1, or a pharmaceutically acceptable salt thereof, wherein each R^{A} is independently selected from 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, each of which is optionally substituted with 1 or 2 independently selected R^{AG} substituents.

17. The compound of claim 1, or a pharmaceutically acceptable salt thereof, wherein R^{A} is phenyl.

18. The compound of claim 1, or a pharmaceutically acceptable salt thereof, wherein R^{A} is C₁₋₆ aliphatic optionally substituted with 1, 2, 3, 4, 5, or 6 R^{AG} substituents.

19. The compound of claim 1, or a pharmaceutically acceptable salt thereof, wherein R^{A} is C₁₋₂ aliphatic optionally substituted with 1, 2, 3, 4, or 5 R^{AG} substituents.

20. The compound of claim 19, or a pharmaceutically acceptable salt thereof, wherein R^{A} is —CH═CH₂.

21. The compound of claim 1, or a pharmaceutically acceptable salt thereof, wherein each R^{A} is independently selected from methyl, ethyl, n-propyl, isopropyl, etheneyl, methoxy, methoxyethoxy, dimethylamino, dimethylaminocarbonyl, azetidinylcarbonyl, (hydroxyazetidinyl)carbonyl, cyclopropyl, cyclopentyl, phenyl, pyridyl, pyrimidinyl, tetrahydrofuranyl, tetrahydropyranyl, azetidinyl, pyrrolidinyl, piperidinyl, tetrahydro-2H-pyranyl, morpholinyl, tetrahydrofuranyl, piperazinyl, 5,6-dihydro-[1,2,4]triazolo[1,5-a]pyrazin-7(8H)-yl, wherein the methyl, ethyl, n-propyl, isopropyl, etheneyl, cyclopropyl, cyclopentyl, phenyl, pyridyl, pyrimidinyl, tetrahydrofuranyl, tetrahydropyranyl, azetidinyl, pyrrolidinyl, piperidinyl, tetrahydro-2H-pyranyl, morpholinyl, tetrahydrofuranyl, piperazinyl, and 5,6-dihydro-[1,2,4]triazolo[1,5-a]pyrazin-7(8H)-yl are each optionally substituted with 1, 2, 3, or 4 independently selected R^{AG} substituents.

22. The compound of claim 1, or a pharmaceutically acceptable salt thereof, wherein each R^{A} is independently selected from methyl, ethyl, n-propyl, isopropyl, etheneyl, methoxy, methoxyethoxy, dimethylamino, cyclopropyl, cyclopentyl, phenyl, methylphenyl, pyridyl, fluoropyridyl, methylpyridyl, pyrimidinyl, tetrahydrofuranyl, and tetrahydropyranyl.

23. The compound of claim 1, or a pharmaceutically acceptable salt thereof, wherein: 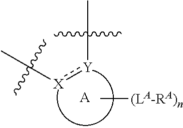
is

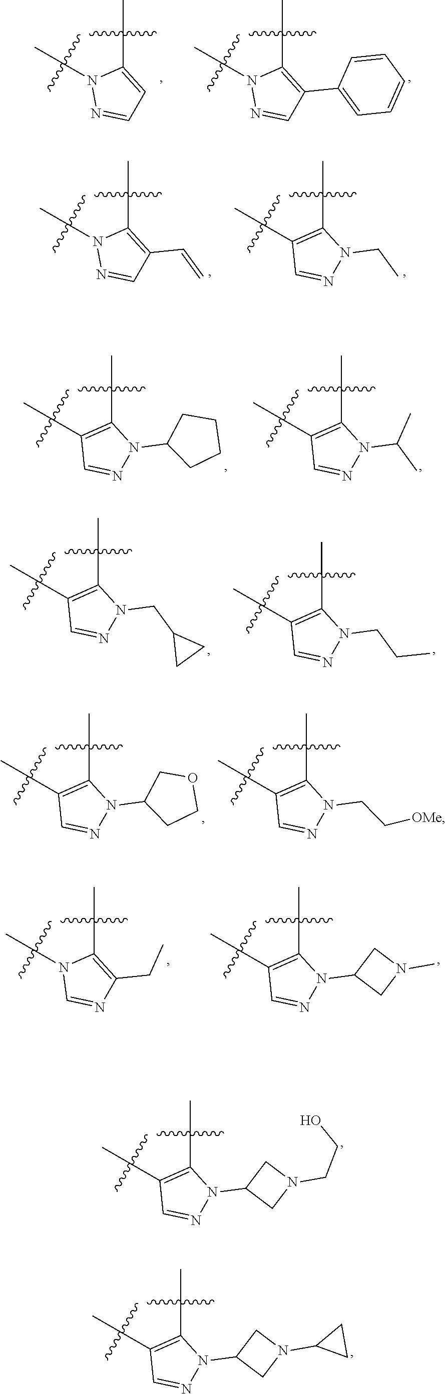

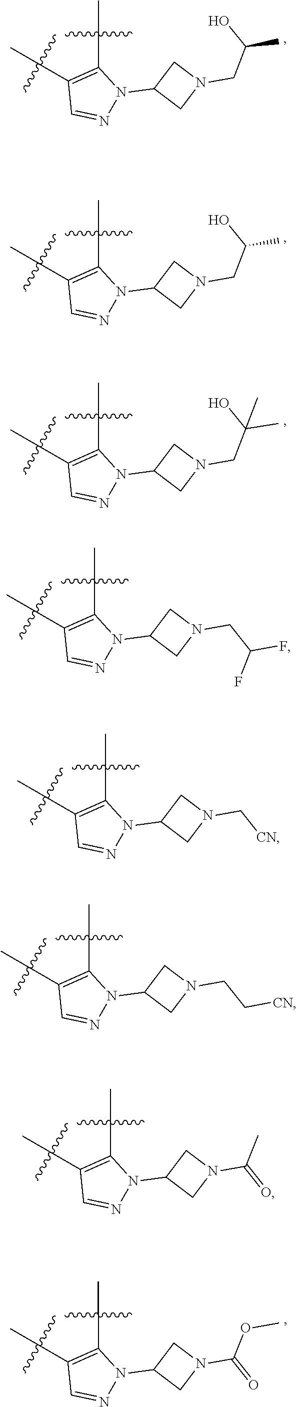

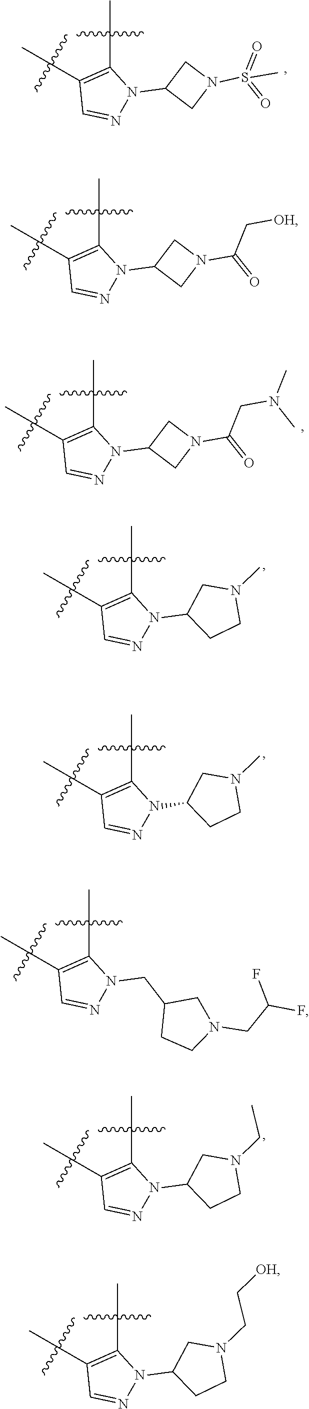

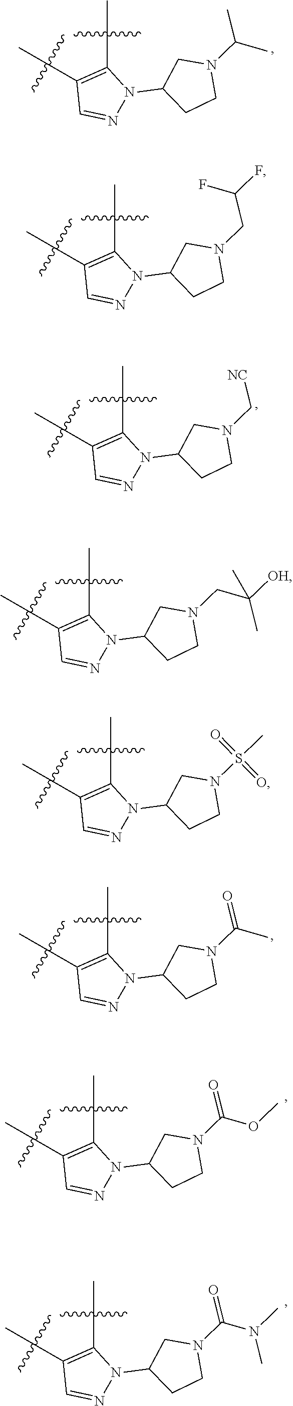

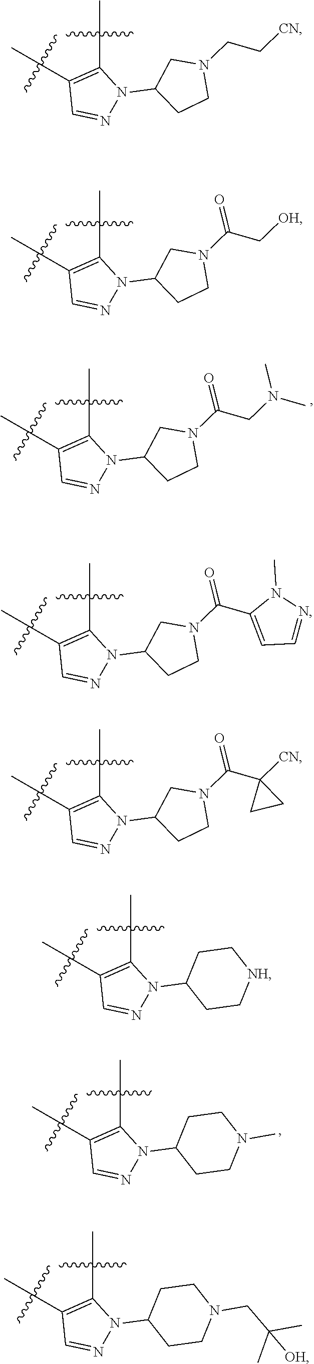

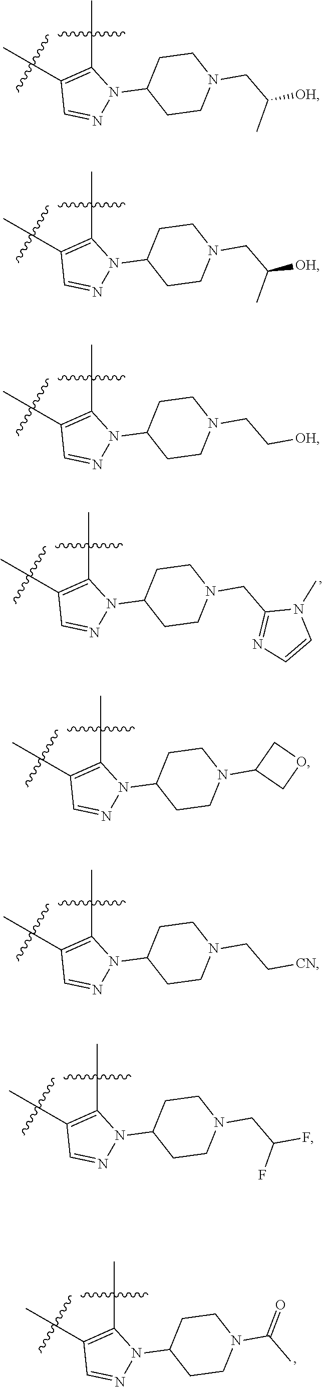

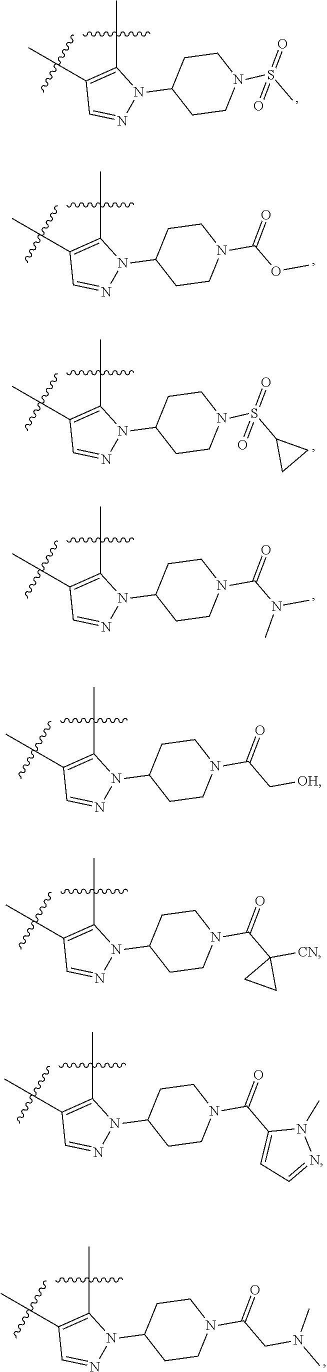

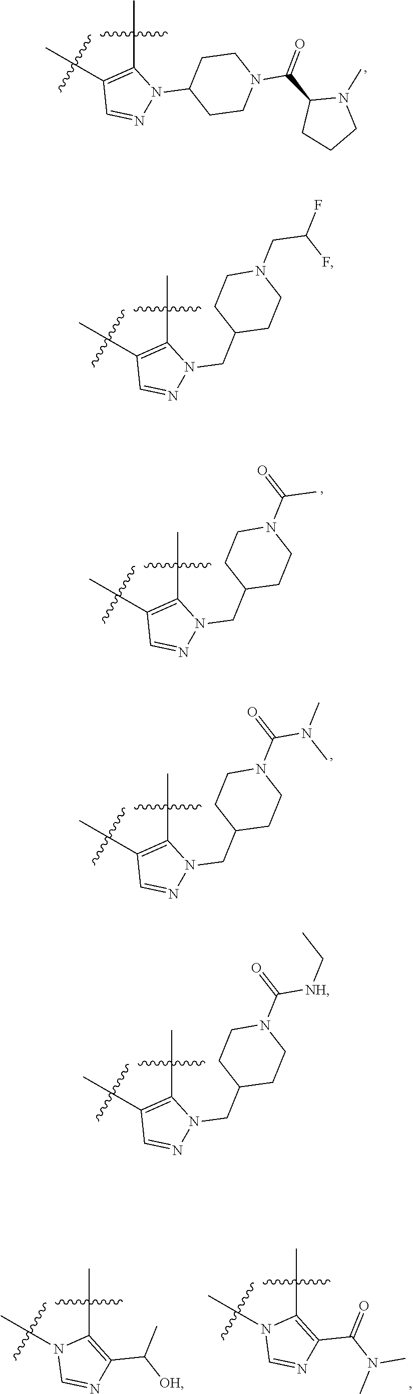

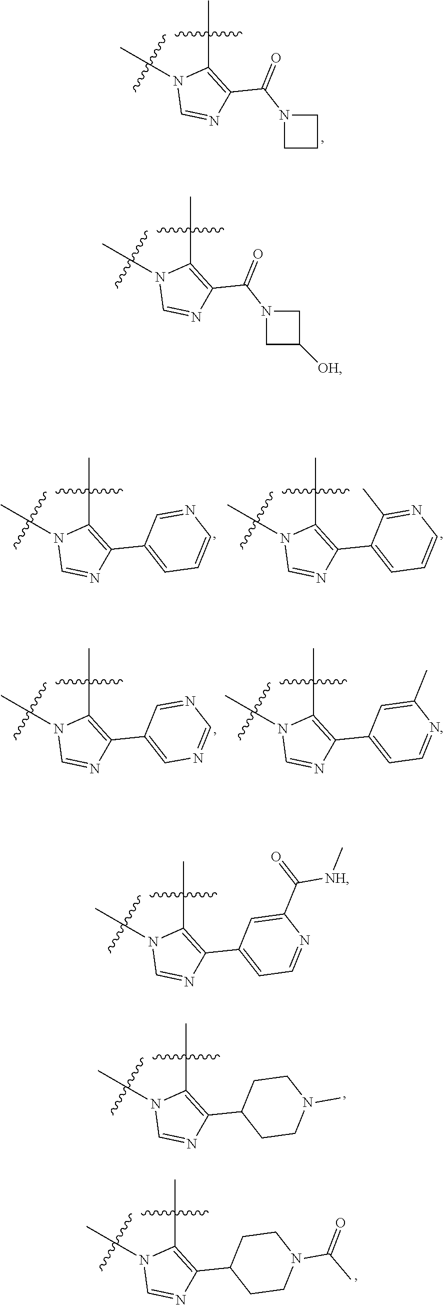

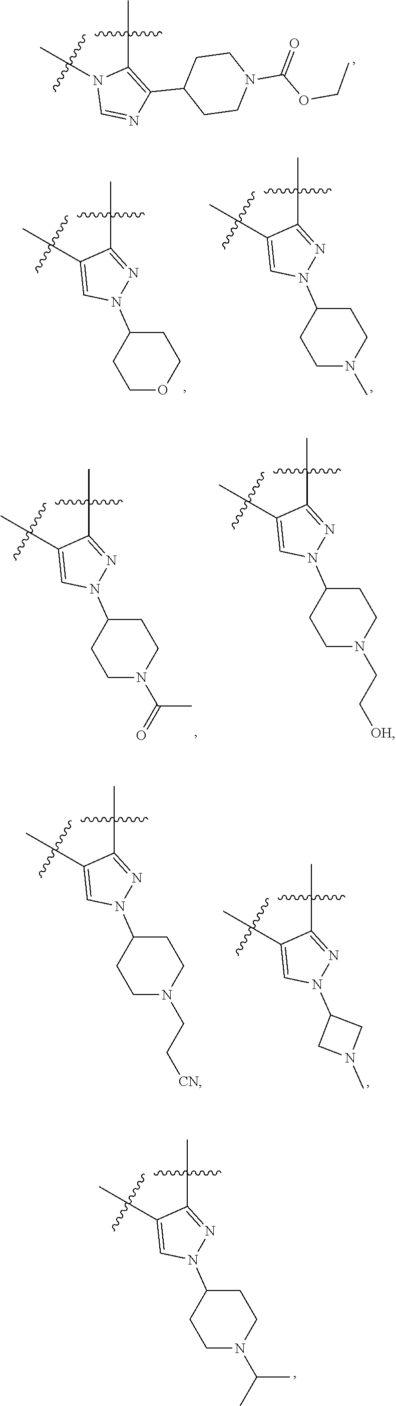

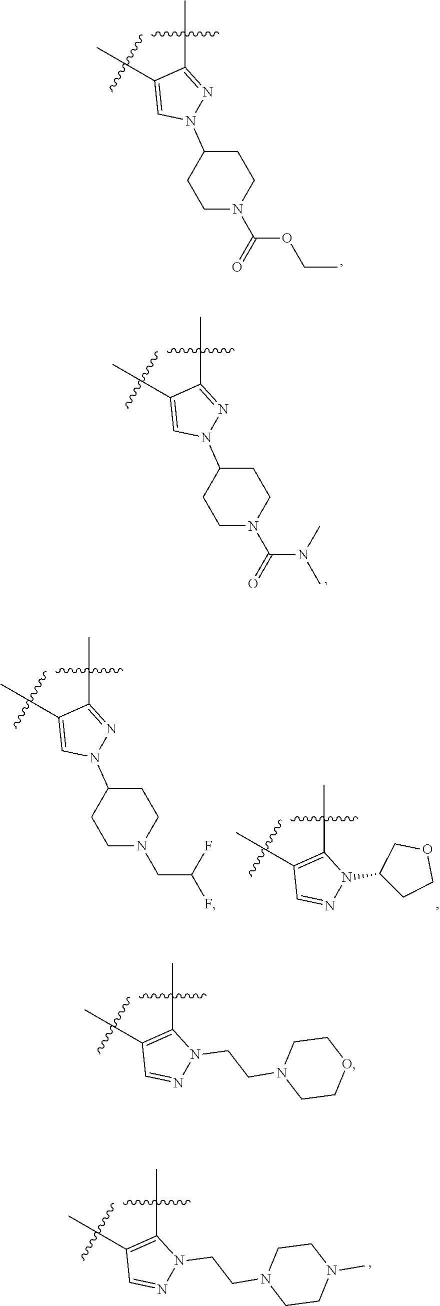 [SMILES: *c1cn(C2CCN(C(=O)OCC)CC2)nc1* ; *c1cn(C2CCN(C(=O)N(C)C)CC2)nc1* ; *c1cn(C2CCN(CC(F)F)CC2)nc1* ; *c1cnn([C@H]2CCOC2)c1* ; *c1cnn(CCN2CCOCC2)c1* ; *c1cnn(CCN2CCN(C)CC2)c1*]

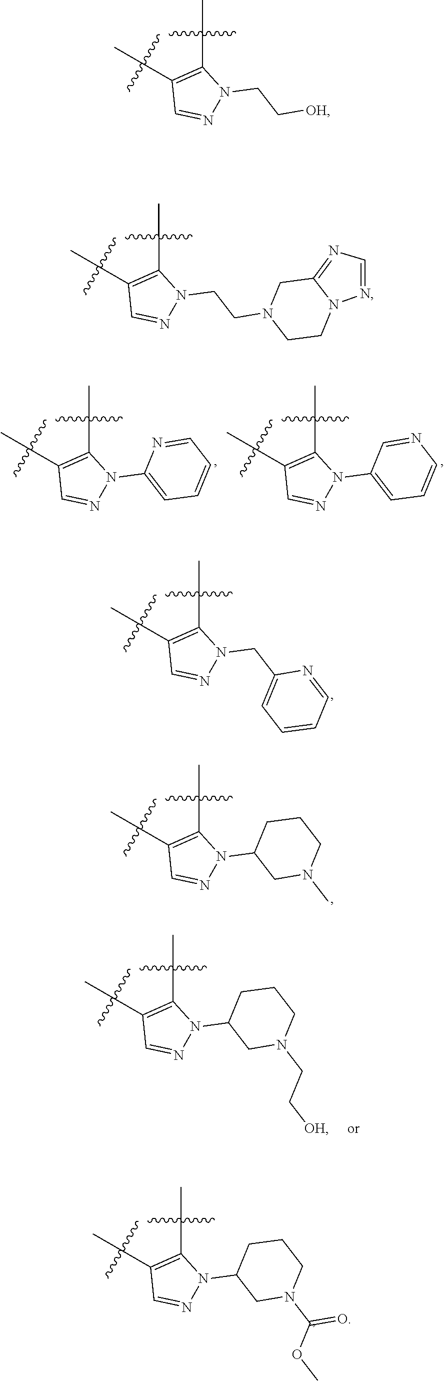

24. The compound of claim 1, or a pharmaceutically acceptable salt thereof, wherein: 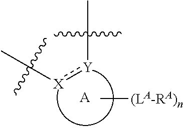
is

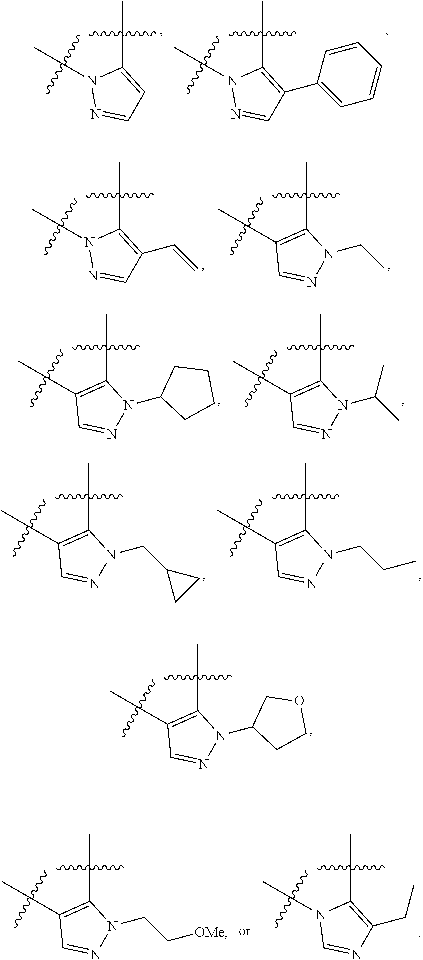

25. The compound of claim 1, or a pharmaceutically acceptable salt thereof, wherein R¹ is an optionally substituted C₁₋₆ aliphatic, 3- to 7-membered saturated or partially unsaturated monocyclic carbocyclyl, or 3- to 7-membered saturated or partially unsaturated monocyclic heterocyclyl having 1-3 heteroatoms independently selected from nitrogen, oxygen, and sulfur.

26. The compound of claim 1, or a pharmaceutically acceptable salt thereof, wherein R¹ is methyl, trideuteromethyl, ethyl, difluoroethyl, hydroxyethyl, cyclopropyl, or tetrahydrofuranyl.

27. The compound of claim 25, or a pharmaceutically acceptable salt thereof, wherein R¹ is methyl.

28. The compound of claim 1, wherein R¹ and one of L^{A}-R^{A} taken together with the atoms to which they are attached form a 3- to 7-membered saturated or partially unsaturated monocyclic heterocyclyl having 1-3 heteroatoms independently selected from nitrogen, oxygen, and sulfur, or 5- to 10-membered saturated or partially unsaturated bicyclic heterocyclyl having 1-4 heteroatoms independently selected from nitrogen, oxygen, and sulfur, wherein each of the 3- to 7-membered monocyclic heterocyclyl and 5- to 10-membered bicyclic heterocyclyl is independently optionally substituted with 1, 2, 3, 4, 5, or 6 R^{AG} substituents.

29. The compound of claim 1, wherein R¹ and one of L^{A}-R^{A} taken together with the atoms to which they are attached form a 6-membered saturated or partially unsaturated monocyclic heterocyclyl having 1-3 heteroatoms independently selected from nitrogen, oxygen, and sulfur, which is optionally substituted with 1 or 2 R^{AG} substituents.

30. The compound of claim 1, or a pharmaceutically acceptable salt thereof, wherein Z is CR².

31. The compound of claim 30, or a pharmaceutically acceptable salt thereof, wherein Z is CH.

32. The compound of claim 1, or a pharmaceutically acceptable salt thereof, wherein R³ is —F, —Cl, —CN, or optionally substituted C₁₋₆ aliphatic.

33. The compound of claim 32, or a pharmaceutically acceptable salt thereof, wherein R³ is optionally substituted C₁₋₆ aliphatic.

34. The compound of claim 33, or a pharmaceutically acceptable salt thereof, wherein R³ is methyl or trifluoromethyl.

35. The compound of claim 1, or a pharmaceutically acceptable salt thereof, wherein U is CR⁴.

36. The compound of claim 35, or a pharmaceutically acceptable salt thereof, wherein U is CH.

37. The compound of claim 1, or a pharmaceutically acceptable salt thereof, wherein L⁵ is optionally substituted bivalent C₁₋₆ aliphatic.

38. The compound of claim 37, or a pharmaceutically acceptable salt thereof, wherein L⁵ is optionally substituted bivalent C₁₋₃ aliphatic.

39. The compound of claim 38, or a pharmaceutically acceptable salt thereof, wherein L⁵ is —CH₂—, —CH(CH₃)—, —CH(CH₂OH)—, or

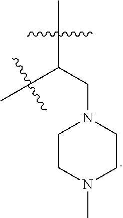 [SMILES: *C(*)CN1CCN(C)CC1]

40. The compound of claim 1, or a pharmaceutically acceptable salt thereof, wherein R^{5A} is —NHR^{5A12} or —OR^{5A11}.

41. The compound of claim 1, or a pharmaceutically acceptable salt thereof, wherein each R^{5A12} is independently 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, each of which is optionally substituted with 1, 2, 3, 4, 5, or 6 independently selected R^{G1} substituents.

42. The compound of claim 1, or a pharmaceutically acceptable salt thereof, wherein each R^{5A12} is independently phenyl or 5- to 6-membered monocyclic heteroaryl having 1-4 heteroatoms independently selected from nitrogen, oxygen, and sulfur, each of which is optionally substituted with 1, 2, 3, 4, 5, or 6 independently selected R^{G1} substituents.

43. The compound of claim 1, or a pharmaceutically acceptable salt thereof, wherein each R^{5A12} is independently phenyl or pyridinyl, each of which is optionally substituted with 1, 2, 3, 4, 5, or 6 independently selected independently selected R^{G1} substituents.

44. The compound of claim 41, or a pharmaceutically acceptable salt thereof, wherein each R^{5A12} is independently phenyl optionally substituted with 1, 2, 3, 4, or 5 independently selected R^{G1} substituents.

45. The compound of claim 1, or a pharmaceutically acceptable salt thereof, wherein a single instance of R^{5A12} is phenyl substituted with 1 or 2 independently selected R^{G1} substituents.

46. The compound of claim 1, or a pharmaceutically acceptable salt thereof, wherein a single instance of R^{5A12} is pyridinyl, which is optionally substituted with 1 or 2 independently selected R^{G1} substituents.

47. The compound of claim 1, or a pharmaceutically acceptable salt thereof, wherein each R^{G1} is independently selected from halogen, C₁₋₆ alkyl, phenyl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl, having 1-4 heteroatoms independently selected from nitrogen, oxygen, and sulfur, 8- to 10-membered bicyclic heteroaryl having 1-4 heteroatoms independently selected from nitrogen, oxygen, and sulfur, —C(O)OR, —C(O)N(R)₂, and —C(O)NR(OR), wherein each C₁₋₆ alkyl, phenyl, 3- to 7-membered saturated or partially unsaturated monocyclic heterocyclyl, 5- to 10-membered saturated or partially unsaturated bicyclic heterocyclyl, 5- to 6-membered monocyclic heteroaryl, and 8- to 10-membered bicyclic heteroaryl is optionally substituted by 1, 2, 3, or 4 independently selected R^{AG1} substituents.

48. The compound of claim 1, or a pharmaceutically acceptable salt thereof, wherein each R is independently selected from hydrogen, C₁₋₆ alkyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur, wherein each 3- to 7-membered saturated or partially unsaturated monocyclic heterocyclyl and 5- to 6-membered monocyclic heteroaryl is optionally substituted by C₁₋₆ alkyl.

49. The compound of claim 1, or a pharmaceutically acceptable salt thereof, wherein each R is independently selected from hydrogen, methyl, methylpiperidinyl, and pyridinyl.

50. The compound of claim 1, or a pharmaceutically acceptable salt thereof, wherein each R^{AG1} is independently selected from halogen, —(CH₂)₀₋₄R^{o}, —(CH₂)₀₋₄OR^{o}, —(CH₂)₀₋₄C(O)NR^{o}₂, —(CH₂)₀₋₄C(O)OR^{o}, —(CH₂)₀₋₄C(O)R^{o}, —O(CH₂)₀₋₄R^{o}, CN, —(CH₂)₀₋₄C(O)NR^{o}₂, and —C(O)N(OR^{o})R^{o}, wherein each R^{o} is independently hydrogen, C₁₋₆ alkyl, or 3- to 6-membered saturated ring having 0-4 heteroatoms independently selected from nitrogen, oxygen, or sulfur.

51. The compound of claim 1, or a pharmaceutically acceptable salt thereof, wherein each R^{AG1} is independently selected from fluoro, methyl, ethyl, hydroxyethyl, —COOH, methylaminocarbonyl, dimethylaminocarbonyl, methylcarbonyl, methoxyaminocarbonyl, cyano, cyclopropyl, hydroxy, methoxy, dimethylamino, dimethylaminomethyl, and tetrahydropyranyl.

52. The compound of claim 1, or a pharmaceutically acceptable salt thereof, wherein each R^{G1} is independently selected from methyl, chloro, pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, tetrazolyl, triazolyl, oxadiazolyl, indazolyl, quinolinyl, piperidinyl, piperazinyl, morpholinyl, isoindolinonyl, hexahydro-3H-oxazolo[3,4-a]pyrazin-3-onyl, 4,5,6,7-tetrahydropyrazolo[1,5-a]pyrazinyl, —C(O)OR, —C(O)N(R)₂, and —C(O)NR(OR), wherein the pyridinyl, pyridinonyl, phenyl, pyrazolyl, pyrimidinyl, piperazinyl, 5,6,7,8-tetrahydro-[1,2,4]triazolo[4,3-a]pyrazinyl, 1,2,4-oxadiazol-5(4H)-onyl, tetrazolyl, triazolyl, oxadiazolyl, indazolyl, quinolinyl, piperidinyl, piperazinyl, morpholinyl, isoindolinonyl, hexahydro-3H-oxazolo[3,4-a]pyrazin-3-onyl, 4,5,6,7-tetrahydropyrazolo[1,5-a]pyrazinyl, are each optionally substituted by 1 or 2 R^{AG1} substituents independently selected from fluoro, methyl, ethyl, hydroxyethyl, —COOH, methylaminocarbonyl, dimethylaminocarbonyl, methylcarbonyl, methoxyaminocarbonyl, cyano, cyclopropyl, hydroxy, methoxy, dimethylamino, dimethylaminomethyl, and tetrahydropyranyl; and
each R is independently selected from hydrogen, methyl, methylpiperidinyl, and pyridinyl.

53. The compound of claim 1, or a pharmaceutically acceptable salt thereof, wherein each R^{G1} is independently selected from halogen, C₁₋₆ alkyl, and —C(O)OR.

54. The compound of claim 1, or a pharmaceutically acceptable salt thereof, wherein each R^{G1} is independently selected from fluoro, chloro, methyl, and —C(O)OH.

55. The compound of claim 44, or a pharmaceutically acceptable salt thereof, wherein a single instance of R^{5A12} is phenyl substituted with 1 R^{G1} substituent.

56. The compound of claim 1, or a pharmaceutically acceptable salt thereof, wherein R^{G1} is —C(O)OR.

57. The compound of claim 56, or a pharmaceutically acceptable salt thereof, wherein R^{G1} is —C(O)OH.

58. The compound of claim 1, or a pharmaceutically acceptable salt thereof, wherein n is 0 or 1.

59. The compound of claim 1, wherein the compound of Formula I is a compound of Formula IV: 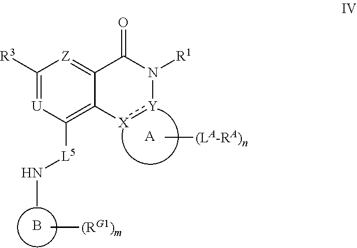
or a pharmaceutically acceptable salt thereof, wherein:
Ring B is a 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; and
m is 0, 1, 2, 3, 4, 5, or 6.

60. The compound of claim 59, or a pharmaceutically acceptable salt thereof, wherein:
L⁵ is a bivalent C₁₋₃ aliphatic which is optionally substituted with 1, 2, 3, or 4 independently selected R^{L5A} substituents;
or L⁵ and one of L^{A}-R^{A} taken together with the atoms to which they are attached form a 3- to 16-membered saturated or partially unsaturated monocyclic carbocyclyl, 5- to 16-membered saturated or partially unsaturated bicyclic carbocyclyl, 3- to 16-membered saturated or partially unsaturated monocyclic heterocyclyl having 1-3 heteroatoms independently selected from nitrogen, oxygen, and sulfur, or 5- to 16-membered saturated or partially unsaturated bicyclic heterocyclyl having 1-4 heteroatoms independently selected from nitrogen, oxygen, and sulfur, wherein each of the 3- to 16-membered monocyclic carbocyclyl, 5- to 16-membered bicyclic carbocyclyl, 3- to 16-membered monocyclic heterocyclyl, and 5- to 16-membered bicyclic heterocyclyl is independently optionally substituted with 1, 2, 3, 4, 5, or 6 R^{AG} substituents.

61. The compound of claim 1, wherein the compound is a compound of Formula V: 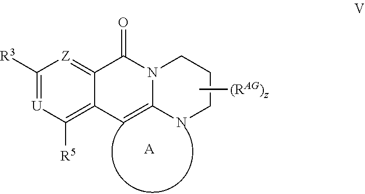
or a pharmaceutically acceptable salt thereof, wherein z is 0, 1, 2, or 3.

62. The compound of claim 1, wherein the compound is of Formula VI: 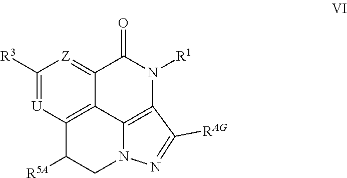
or a pharmaceutically acceptable salt thereof.

63. The compound of claim 1, wherein the compound is of Formula VII: 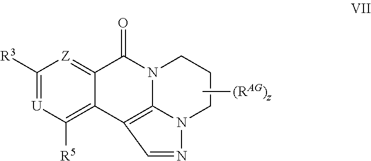
or a pharmaceutically acceptable salt thereof, wherein z is 0, 1, 2, or 3.

64. The compound of claim 1, wherein the compound is selected from:
2-((1-(4,7-dimethyl-5-oxo-4,5-dihydropyrazolo[1,5-a]quinazolin-9-yl)ethyl)amino)benzoic acid;
2-((1-(4,7-dimethyl-5-oxo-3-phenyl-4,5-dihydropyrazolo[1,5-a]quinazolin-9-yl)ethyl)amino)benzoic acid; and
2-((1-(4,7-dimethyl-5-oxo-3-vinyl-4,5-dihydropyrazolo[1,5-a]quinazolin-9-yl)ethyl)amino)benzoic acid;
or a pharmaceutically acceptable salt thereof.

65. The compound of claim 1, wherein the compound is selected from:
2-((1-(4,7-dimethyl-5-oxo-4,5-dihydroimidazo[1,5-a]quinazolin-9-yl)ethyl)amino)benzoic acid;
2-((1-(3-ethyl-4,7-dimethyl-5-oxo-4,5-dihydroimidazo[1,5-a]quinazolin-9-yl)ethyl)amino)benzoic acid;
2-((1-(4,7-dimethyl-5-oxo-3-(pyridin-2-yl)-4,5-dihydroimidazo[1,5-a]quinazolin-9-yl)ethyl)amino)benzoic acid;
2-((1-(3,4,7-trimethyl-5-oxo-4,5-dihydroimidazo[1,5-a]quinazolin-9-yl)ethyl)amino)benzoic acid;
6-methyl-3-((1-(3,4,7-trimethyl-5-oxo-4,5-dihydroimidazo[1,5-a]quinazolin-9-yl)ethyl)amino)picolinic acid;
2-((1-(7-chloro-3-ethyl-4-methyl-5-oxo-4,5-dihydroimidazo[1,5-a]quinazolin-9-yl)ethyl)amino)benzoic acid;
2-((1-(7-chloro-4-methyl-5-oxo-4,5-dihydroimidazo[1,5-a]quinazolin-9-yl)ethyl)amino)benzoic acid;
2-((1-(7-chloro-3,4-dimethyl-5-oxo-4,5-dihydroimidazo[1,5-a]quinazolin-9-yl)ethyl)amino)benzoic acid;
2-((1-(7-chloro-4-ethyl-5-oxo-4,5-dihydroimidazo[1,5-a]quinazolin-9-yl)ethyl)amino)benzoic acid;
2-((1-(7-chloro-3,4-diethyl-5-oxo-4,5-dihydroimidazo[1,5-a]quinazolin-9-yl)ethyl)amino)benzoic acid;
2-((1-(4,7-dimethyl-5-oxo-4,5-dihydroimidazo[1,2-a]quinazolin-9-yl)ethyl)amino)benzoic acid;
2-((1-(4,7-dimethyl-5-oxo-4,5-dihydro-[1,2,4]triazolo[1,5-a]quinazolin-9-yl)ethyl)amino)benzoic acid;
2-((1-(3,4,7-trimethyl-5-oxo-4,5-dihydro-3H-pyrazolo[3,4-c]isoquinolin-9-yl)ethyl)amino)benzoic acid;
6-chloro-3-((1-(3,4,7-trimethyl-5-oxo-4,5-dihydro-3H-pyrazolo[3,4-c]isoquinolin-9-yl)ethyl)amino)picolinic acid;
2-((1-(3-ethyl-4,7-dimethyl-5-oxo-4,5-dihydro-3H-pyrazolo[3,4-c]isoquinolin-9-yl)ethyl)amino)benzoic acid;
6-chloro-3-((1-(3-ethyl-4,7-dimethyl-5-oxo-4,5-dihydro-3H-pyrazolo[3,4-c]isoquinolin-9-yl)ethyl)amino)picolinic acid;
3-((1-(3-ethyl-4,7-dimethyl-5-oxo-4,5-dihydro-3H-pyrazolo[3,4-c]isoquinolin-9-yl)ethyl)amino)-6-methylpicolinic acid;
2-((1-(4-ethyl-3,7-dimethyl-5-oxo-4,5-dihydro-3H-pyrazolo[3,4-c]isoquinolin-9-yl)ethyl)amino)benzoic acid;
6-chloro-3-((1-(4-ethyl-3,7-dimethyl-5-oxo-4,5-dihydro-3H-pyrazolo[3,4-c]isoquinolin-9-yl)ethyl)amino)picolinic acid;
2-((1-(3,4-diethyl-7-methyl-5-oxo-4,5-dihydro-3H-pyrazolo[3,4-c]isoquinolin-9-yl)ethyl)amino)benzoic acid;
2-((1-(3,4-diethyl-7-methyl-5-oxo-4,5-dihydro-3H-pyrazolo[3,4-c]isoquinolin-9-yl)ethyl)amino)-5-fluorobenzoic acid;
3-((1-(3,4-diethyl-7-methyl-5-oxo-4,5-dihydro-3H-pyrazolo[3,4-c]isoquinolin-9-yl)ethyl)amino)-6-methylpicolinic acid;
2-((1-(4,7-dimethyl-5-oxo-2-propyl-4,5-dihydro-2H-pyrazolo[3,4-c]isoquinolin-9-yl)ethyl)amino)benzoic acid;
2-((1-(4,7-dimethyl-5-oxo-2-(tetrahydro-2H-pyran-4-yl)-4,5-dihydro-2H-pyrazolo[3,4-c]isoquinolin-9-yl)ethyl)amino)benzoic acid;
2-((1-(4,7-dimethyl-5-oxo-2-phenethyl-4,5-dihydro-2H-pyrazolo[3,4-c]isoquinolin-9-yl)ethyl)amino)benzoic acid;
2-((1-(2-(2-fluoropyridin-4-yl)-4,7-dimethyl-5-oxo-4,5-dihydro-2H-pyrazolo[3,4-c]isoquinolin-9-yl)ethyl)amino)benzoic acid;
2-((1-(4,7-dimethyl-5-oxo-2-(pyrimidin-5-yl)-4,5-dihydro-2H-pyrazolo[3,4-c]isoquinolin-9-yl)ethyl)amino)benzoic acid;
6-chloro-3-((1-(4,7-dimethyl-5-oxo-2-(o-tolyl)-4,5-dihydro-2H-pyrazolo[3,4-c]isoquinolin-9-yl)ethyl)amino)picolinic acid;
2-[1-(3-methoxy-5,8-dimethyl-6-oxo-benzo[c][1,8]naphthyridin-10-yl)ethylamino]benzoic acid;
2-[1-[3-(dimethylamino)-5,8-dimethyl-6-oxo-benzo[c][1,8]naphthyridin-10-yl]ethylamino]benzoic acid;
2-[1-[3-(2-methoxyethoxy)-5,8-dimethyl-6-oxo-benzo[c][1,8]naphthyridin-10-yl]ethylamino]benzoic acid; and
2-((1-(7-chloro-3,4-dimethyl-5-oxo-4,5-dihydro-3H-pyrazolo[3,4-c]isoquinolin-9-yl)ethyl)amino)benzoic acid;
or a pharmaceutically acceptable salt thereof.

66. The compound of claim 1, wherein the compound is selected from:
2-((1-(8-methyl-6-oxo-4,5-dihydro-3H,6H-2,2a,5a-triazaaceanthrylen-10-yl)ethyl)amino)benzoic acid;
6-methyl-3-((1-(8-methyl-6-oxo-4,5-dihydro-3H,6H-2,2a,5a-triazaaceanthrylen-10-yl)ethyl)amino)picolinic acid;
2-((1-(3,8-dimethyl-6-oxo-4,5-dihydro-3H,6H-2,2a,5a-triazaaceanthrylen-10-yl)ethyl)amino)benzoic acid; and
6-chloro-3-((1-(3,8-dimethyl-6-oxo-4,5-dihydro-3H,6H-2,2a,5a-triazaaceanthrylen-10-yl)ethyl)amino)picolinic acid;
or a pharmaceutically acceptable salt thereof.

67. The compound of claim 1, wherein the compound is selected from:
2-ethyl-3,6-dimethyl-8-((6-methyl-[2,4′-bipyridin]-3-yl)amino)-8,9-dihydrobenzo[de]pyrazolo[4,5,1-ij][1,7]naphthyridin-4(3H)-one;
9-(1-((6-chloro-1′-methyl-6′-oxo-1′,6′-dihydro-[2,3′-bipyridin]-3-yl)amino)ethyl)-3-ethyl-4,7-dimethyl-3,4-dihydro-5H-pyrazolo[3,4-c]isoquinolin-5-one;
4-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)-2-fluoro-N-methylbenzamide;
9-(1-((6-chloro-2-(pyrimidin-4-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(pyrimidin-5-yl)pyridin-3-yl)amino)ethyl)-3-ethyl-4,7-dimethyl-3,4-dihydro-5H-pyrazolo[3,4-c]isoquinolin-5-one;
9-(1-((6-chloro-3′-fluoro-[2,4′-bipyridin]-3-yl)amino)ethyl)-3-ethyl-4,7-dimethyl-3,4-dihydro-5H-pyrazolo[3,4-c]isoquinolin-5-one;
4-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)-N,N-dimethylbenzamide;
4-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)benzoic acid;
9-(1-((6-chloro-2-(1-methyl-1H-pyrazol-5-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(1,5-dimethyl-1H-pyrazol-4-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(1H-pyrazol-4-yl)pyridin-3-yl)amino)ethyl)-3-ethyl-4,7-dimethyl-3,4-dihydro-5H-pyrazolo[3,4-c]isoquinolin-5-one;
3-ethyl-4,7-dimethyl-9-(1-((6-methyl-2-(1-methyl-1H-pyrazol-4-yl)pyridin-3-yl)amino)ethyl)-3,4-dihydro-5H-pyrazolo[3,4-c]isoquinolin-5-one;
3-ethyl-4,7-dimethyl-9-(1-((6-methyl-2-(1H-pyrazol-4-yl)pyridin-3-yl)amino)ethyl)-3,4-dihydro-5H-pyrazolo[3,4-c]isoquinolin-5-one;
3-ethyl-4,7-dimethyl-9-(1-((6-methyl-2-(1-methyl-1H-pyrazol-5-yl)pyridin-3-yl)amino)ethyl)-3,4-dihydro-5H-pyrazolo[3,4-c]isoquinolin-5-one;
4-(3-((1-(3-ethyl-4,7-dimethyl-5-oxo-4,5-dihydro-3H-pyrazolo[3,4-c]isoquinolin-9-yl)ethyl)amino)-6-methylpyridin-2-yl)-2-fluoro-N-methylbenzamide;
3-ethyl-9-(1-((2-(1-(2-hydroxyethyl)-1H-pyrazol-4-yl)phenyl)amino)ethyl)-4,7-dimethyl-3,4-dihydro-5H-pyrazolo[3,4-c]isoquinolin-5-one;
9-(1-((2-(4-acetylpiperazin-1-yl)phenyl)amino)ethyl)-3-ethyl-4,7-dimethyl-3,4-dihydro-5H-pyrazolo[3,4-c]isoquinolin-5-one;
9-(1-((2-(5,6-dihydro-[1,2,4]triazolo[4,3-a]pyrazin-7(8H)-yl)phenyl)amino)ethyl)-3-ethyl-4,7-dimethyl-3,4-dihydro-5H-pyrazolo[3,4-c]isoquinolin-5-one;
3-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(4-(2,2-difluoroethyl)-3-ethyl-7-methyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3-cyclopentyl-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3-isopropyl-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(7-chloro-3-ethyl-4-methyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(7-chloro-3-(cyclopropylmethyl)-4-methyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(4,7-dimethyl-5-oxo-3-propyl-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(4,7-dimethyl-5-oxo-3-(tetrahydrofuran-3-yl)-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3-(2-methoxyethyl)-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3-(cyclopropylmethyl)-4,7-dimethyl-5-oxo-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3-ethyl-4-methyl-5-oxo-7-(trifluoromethyl)-4,5-dihydro-3H-pyrazolo[3,4-c]isoquinolin-9-yl)ethyl)amino)pyridin-2-yl)-1,2,4-oxadiazol-5(4H)-one;
3-(6-chloro-3-((1-(3,8-dimethyl-6-oxo-4,5-dihydro-3H,6H-2,2a,5a-triazaaceanthrylen-10-yl)ethyl)amino)pyridin-2-yl)-1,2,4-oxadiazol-5(4H)-one;
3-(6-chloro-3-((1-(3-ethyl-4,7-dimethyl-5-oxo-4,5-dihydroimidazo[1,5-a]quinazolin-9-yl)ethyl)amino)pyridin-2-yl)-1,2,4-oxadiazol-5(4H)-one;
9-(1-((2-(1H-tetrazol-5-yl)pyridin-3-yl)amino)ethyl)-3-ethyl-4,7-dimethyl-3,4-dihydro-5H-pyrazolo[3,4-c]isoquinolin-5-one;
6-chloro-3-((1-(3-ethyl-4,7-dimethyl-5-oxo-4,5-dihydro-3H-pyrazolo[3,4-c]isoquinolin-9-yl)ethyl)amino)-N-(1-methylpiperidin-4-yl)picolinamide;
6-chloro-3-((1-(3-ethyl-4,7-dimethyl-5-oxo-4,5-dihydro-3H-pyrazolo[3,4-c]isoquinolin-9-yl)ethyl)amino)-N-(pyridin-4-yl)picolinamide;
6-chloro-3-((1-(3-ethyl-4,7-dimethyl-5-oxo-4,5-dihydro-3H-pyrazolo[3,4-c]isoquinolin-9-yl)ethyl)amino)-N-methoxypicolinamide;
9-(1-((6-chloro-3′-fluoro-[2,4′-bipyridin]-3-yl)amino)-2-hydroxyethyl)-3-ethyl-4,7-dimethyl-3,4-dihydro-5H-pyrazolo[3,4-c]isoquinolin-5-one;
9-(1-((6-chloro-2-(1-methyl-1H-pyrazol-4-yl)pyridin-3-yl)amino)-2-hydroxyethyl)-3-ethyl-4,7-dimethyl-3,4-dihydro-5H-pyrazolo[3,4-c]isoquinolin-5-one;
9-(1-((6-chloro-2-(1,5-dimethyl-1H-pyrazol-4-yl)pyridin-3-yl)amino)-2-hydroxyethyl)-3-ethyl-4,7-dimethyl-3,4-dihydro-5H-pyrazolo[3,4-c]isoquinolin-5-one; and
9-(1-((2-(4-acetylpiperazin-1-yl)-6-chloropyridin-3-yl)amino)ethyl)-3-ethyl-4,7-dimethyl-3,4-dihydro-5H-pyrazolo[3,4-c]isoquinolin-5-one;
or a pharmaceutically acceptable salt thereof.

68. The compound of claim 1, wherein the compound is selected from:
9-(1-((6-chloro-2-(1-methyl1H-pyrazol-4-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-2-(1-(2-hydroxyethyl)-1H-1,2,4-triazol-3-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-2-(1H-pyrazol-4-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-1′-methyl-6′-oxo-1′,6′-dihydro-[2,3′-bipyridin]-3-yl)amino)ethyl)-4,7-dimethyl-3-(1-methylazetidin-3-yl)-3,4-dihydro-5H-pyrazolo[3,4-c]isoquinolin-5-one;
5-(5-chloro-2-((1-(4,7-dimethyl-3-(1-methylazetidin-3-yl)-5-oxo-4,5-dihydro-3H-pyrazolo[3,4-c]isoquinolin-9-yl)ethyl)amino)phenyl)-N-methylpicolinamide;
9-(1-((6-chloro-2-(2-methyl-1-oxoisoindolin-5-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-2-(1-methyl-1H-indazol-5-yl)pyridin-3-yl)amino)ethyl)-4,7-dimethyl-3-(1-methylazetidin-3-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2-hydroxyethyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-cyclopropylazetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-((S)-2-hydroxypropyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-((R)-2-hydroxypropyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2-hydroxy-2-methylpropyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2,2-difluoroethyl) azetidin-3-yl)-4,7-dimethyl-3,4-dihydro-5H-pyrazolo[3,4-c]isoquinolin-5-one;
2-(3-(9-(1-((6-chloro-2-(1-methyl-1H-1,2,4-triazol-3-yl)pyridin-3-yl)amino)ethyl)-4,7-dimethyl-5-oxo-4,5-dihydro-3H-pyrazolo[3,4-c]isoquinolin-3-yl) azetidin-1-yl) acetonitrile;
3-(3-(9-(1-((6-chloro-2-(1-methyl-1H-1,2,4-triazol-3-yl)pyridin-3-yl)amino)ethyl)-4,7-dimethyl-5-oxo-4,5-dihydro-3H-pyrazolo[3,4-c]isoquinolin-3-yl) azetidin-1-yl)propanenitrile;
3-(1-acetylazetidin-3-yl)-9-(1-((6-chloro-2-(1-methyl-1H-1,2,4-triazol-3-yl)pyridin-3-yl)amino)ethyl)-4,7-dimethyl-3,4-dihydro-5H-pyrazolo[3,4-c]isoquinolin-5-one;
methyl 3-(9-(1-((6-chloro-2-(1-methyl-1H-1,2,4-triazol-3-yl)pyridin-3-yl)amino)ethyl)-4,7-dimethyl-5-oxo-4,5-dihydro-3H-pyrazolo[3,4-c]isoquinolin-3-yl) azetidine-1-carboxylate;
9-(1-((6-chloro-2-(1-methyl-1H-1,2,4-triazol-3-yl)pyridin-3-yl)amino)ethyl)-4,7-dimethyl-3-(1-(methylsulfonyl) azetidin-3-yl)-3,4-dihydro-5H-pyrazolo[3,4-c]isoquinolin-5-one;
3-(1-acetylazetidin-3-yl)-9-(1-((6-chloro-1′-methyl-6′-oxo-1′,6′-dihydro-[2,3′-bipyridin]-3-yl)amino)ethyl)-4,7-dimethyl-3,4-dihydro-5H-pyrazolo[3,4-c]isoquinolin-5-one;
3-((1-(3-(1-acetylazetidin-3-yl)-4,7-dimethyl-5-oxo-4,5-dihydro-3H-pyrazolo[3,4-c]isoquinolin-9-yl)ethyl)amino)-6-chloro-N-methyl-[2,3′-bipyridine]-6′-carboxamide;
3-(1-acetylazetidin-3-yl)-9-(1-((6-chloro-2-(1-(2-hydroxyethyl)-1H-1,2,4-triazol-3-yl)pyridin-3-yl)amino)ethyl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2-hydroxyacetyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(dimethylglycyl) azetidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-7-methyl-4-(methyl-d3)-3-((S)-1-methylpyrrolidin-3-yl)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-((1-(2,2-difluoroethyl)piperidin-4-yl)methyl)-4,7-dimethyl-3,4-dihydro-5H-pyrazolo[3,4-c]isoquinolin-5-one;
3-((1-acetylpiperidin-4-yl)methyl)-9-(1-((6-chloro-2-(1-methyl-1H-pyrazol-4-yl)pyridin-3-yl)amino)ethyl)-4,7-dimethyl-3,4-dihydro-5H-pyrazolo[3,4-c]isoquinolin-5-one;
4-((9-(1-((6-chloro-2-(1-methyl-1H-pyrazol-4-yl)pyridin-3-yl)amino)ethyl)-4,7-dimethyl-5-oxo-4,5-dihydro-3H-pyrazolo[3,4-c]isoquinolin-3-yl)methyl)-N,N-dimethylpiperidine-1-carboxamide;
4-((9-(1-((6-chloro-3′-fluoro-[2,4′-bipyridin]-3-yl)amino)ethyl)-4,7-dimethyl-5-oxo-4,5-dihydro-3H-pyrazolo[3,4-c]isoquinolin-3-yl)methyl)-N-ethylpiperidine-1-carboxamide;
9-(1-((6-chloro-3′-fluoro-[2,4′-bipyridin]-3-yl)amino)ethyl)-3-((1-(2,2-difluoroethyl) pyrrolidin-3-yl)methyl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-hydroxyethyl)-4,7-dimethylimidazo[1,5-a]quinazolin-5(4H)-one;
9-(1-((6-chloro-2-(1-methyl-1H-1,2,4-triazol-3-yl)pyridin-3-yl)amino)ethyl)-4,7-dimethyl-3-(pyridin-3-yl) imidazo[1,5-a]quinazolin-5(4H)-one;
9-(1-((6-chloro-2-(1-methyl-1H-1,2,4-triazol-3-yl)pyridin-3-yl)amino)ethyl)-4,7-dimethyl-3-(2-methylpyridin-3-yl) imidazo[1,5-a]quinazolin-5(4H)-one;
9-(1-((6-chloro-2-(1-methyl-1H-1,2,4-triazol-3-yl)pyridin-3-yl)amino)ethyl)-4,7-dimethyl-3-(pyrimidin-5-yl) imidazo[1,5-a]quinazolin-5(4H)-one;
9-(1-((6-chloro-2-(1-methyl-1H-1,2,4-triazol-3-yl)pyridin-3-yl)amino)ethyl)-4,7-dimethyl-3-(2-methylpyridin-4-yl) imidazo[1,5-a]quinazolin-5(4H)-one;
9-(1-((6-chloro-2-(1-methyl-1H-pyrazol-4-yl)pyridin-3-yl)amino)ethyl)-4,7-dimethyl-3-(2-methylpyridin-3-yl) imidazo[1,5-a]quinazolin-5(4H)-one;
9-(1-((6-chloro-2-(1-methyl-1H-pyrazol-4-yl)pyridin-3-yl)amino)ethyl)-4,7-dimethyl-3-(pyrimidin-5-yl) imidazo[1,5-a]quinazolin-5(4H)-one;
4-(9-(1-((6-chloro-2-(1-methyl-1H-pyrazol-4-yl)pyridin-3-yl)amino)ethyl)-4,7-dimethyl-5-oxo-4,5-dihydroimidazo[1,5-a]quinazolin-3-yl)-N-methylpicolinamide;
9-(1-((6-chloro-2-(1-methyl-1H-1,2,4-triazol-3-yl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-(1-methyl-1H-pyrazol-4-yl)pyridin-3-yl)amino)ethyl)-N,N,4,7-tetramethyl-5-oxo-4,5-dihydroimidazo[1,5-a]quinazoline-3-carboxamide;
3-(azetidine-1-carbonyl)-9-(1-((6-chloro-2-(1-methyl-1H-pyrazol-4-yl)pyridin-3-yl)amino)ethyl)-4,7-dimethylimidazo[1,5-a]quinazolin-5(4H)-one;
9-(1-((6-chloro-2-(1-methyl-1H-pyrazol-4-yl)pyridin-3-yl)amino)ethyl)-3-(3-hydroxyazetidine-1-carbonyl)-4,7-dimethylimidazo[1,5-a]quinazolin-5(4H)-one;
9-(1-((6-chloro-2-(1-methyl-1H-1,2,4-triazol-3-yl)pyridin-3-yl)amino)ethyl)-4-(2,2-difluoroethyl)-N,N,7-trimethyl-5-oxo-4,5-dihydroimidazo[1,5-a]quinazoline-3-carboxamide;
9-(1-((6-chloro-2-(3-methyl-1H-pyrazol-4-yl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-(4-cyano-2-fluorophenyl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-(4-cyanophenyl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-morpholinopyridin-3-yl)amino)ethyl)-N,N,4,7-tetramethyl-5-oxo-4,5-dihydroimidazo[1,5-a]quinazoline-3-carboxamide;
9-(1-((4-fluoro-2-(1-methyl-1H-pyrazol-4-yl)phenyl)amino)ethyl)-N,N,4,7-tetramethyl-5-oxo-4,5-dihydroimidazo[1,5-a]quinazoline-3-carboxamide;
9-(1-((6-chloro-2-(1,5-dimethyl-1H-pyrazol-4-yl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-(1,3-dimethyl-1H-pyrazol-4-yl)pyridin-3-yl)amino)ethyl)-N,N,4,7-tetramethyl-5-oxo-4,5-dihydroimidazo[1,5-a]quinazoline-3-carboxamide;
9-(1-((6-chloro-2-(1-methyl-1H-1,2,4-triazol-3-yl)pyridin-3-yl)amino)ethyl)-4,7-dimethyl-3-(1-methylpiperidin-4-yl) imidazo[1,5-a]quinazolin-5(4H)-one;
3-(1-acetylpiperidin-4-yl)-9-(1-((6-chloro-2-(1-methyl-1H-1,2,4-triazol-3-yl)pyridin-3-yl)amino)ethyl)-4,7-dimethylimidazo[1,5-a]quinazolin-5(4H)-one;
ethyl 4-(9-(1-((6-chloro-2-(1-methyl-1H-1,2,4-triazol-3-yl)pyridin-3-yl)amino)ethyl)-4,7-dimethyl-5-oxo-4,5-dihydroimidazo[1,5-a]quinazolin-3-yl)piperidine-1-carboxylate;
9-(1-((6-chloro-2-(1-methyl-1H-pyrazol-4-yl)pyridin-3-yl)amino)ethyl)-4,7-dimethyl-3-(1-methylpiperidin-4-yl) imidazo[1,5-a]quinazolin-5(4H)-one;
9-(1-((6-chloro-2-(1-methyl-1H-pyrazol-4-yl)pyridin-3-yl)amino)ethyl)-4,7-dimethyl-3-(1-methylpyrrolidin-3-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-methylpyrrolidin-3-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-ethylpyrrolidin-3-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2-hydroxyethyl) pyrrolidin-3-yl)-4,7-dimethyl-3,4-dihydro-5H-pyrazolo[3,4-c]isoquinolin-5-one;
9-(1-((6-chloro-2-(2-methyl-2H-tetrazol-5-yl)pyridin-3-yl)amino)ethyl)-7-methyl-4-(methyl-d3)-3-(1-methylpyrrolidin-3-yl)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-(1-(2-hydroxyethyl) pyrrolidin-3-yl)-7-methyl-4-(methyl-d3)-3,4-dihydro-5H-pyrazolo[3,4-c]isoquinolin-5-one;
9-(1-((6-chloro-3′-fluoro-[2,4′-bipyridin]-3-yl)amino)ethyl)-3-(1-isopropylpyrrolidin-3-yl)-4,7-dimethyl-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-(1-(2,2-difluoroethyl) pyrrolidin-3-yl)-4,7-dimethyl-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-(1-(2,2-difluoroethyl) pyrrolidin-3-yl)-7-methyl-4-(methyl-d3)-3,4-dihydro-5H-pyrazolo[3,4-c]isoquinolin-5-one;
2-(3-(9-(1-((6-chloro-2-(1-methyl-1H-pyrazol-4-yl)pyridin-3-yl)amino)ethyl)-7-methyl-4-(methyl-d3)-5-oxo-4,5-dihydro-3H-pyrazolo[3,4-c]isoquinolin-3-yl) pyrrolidin-1-yl) acetonitrile;
9-(1-((6-chloro-2-(1-methyl-1H-1,2,4-triazol-3-yl)pyridin-3-yl)amino)ethyl)-4-ethyl-3-(1-(2-hydroxyethyl) pyrrolidin-3-yl)-7-methyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4-ethyl-3-(1-(2-hydroxy-2-methylpropyl) pyrrolidin-3-yl)-7-methyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4-ethyl-3-(1-isopropylpyrrolidin-3-yl)-7-methyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4-ethyl-7-methyl-3-(1-(methylsulfonyl) pyrrolidin-3-yl)-3,4-dihydro-5H-pyrazolo[3,4-c]isoquinolin-5-one;
3-(1-acetylpyrrolidin-3-yl)-9-(1-((6-chloro-2-(1-methyl-1H-1,2,4-triazol-3-yl)pyridin-3-yl)amino)ethyl)-4-ethyl-7-methyl-3,4-dihydro-5H-pyrazolo[3,4-c]isoquinolin-5-one;
methyl 3-(9-(1-((6-chloro-2-(1-methyl-1H-1,2,4-triazol-3-yl)pyridin-3-yl)amino)ethyl)-4-ethyl-7-methyl-5-oxo-4,5-dihydro-3H-pyrazolo[3,4-c]isoquinolin-3-yl) pyrrolidine-1-carboxylate;
3-(9-(1-((6-chloro-3′-fluoro-[2,4′-bipyridin]-3-yl)amino)ethyl)-4,7-dimethyl-5-oxo-4,5-dihydro-3H-pyrazolo[3,4-c]isoquinolin-3-yl)-N,N-dimethylpyrrolidine-1-carboxamide;
3-(3-(9-(1-((6-chloro-2-(1-methyl-1H-pyrazol-4-yl)pyridin-3-yl)amino)ethyl)-7-methyl-4-(methyl-d3)-5-oxo-4,5-dihydro-3H-pyrazolo[3,4-c]isoquinolin-3-yl) pyrrolidin-1-yl)propanenitrile;
3-(3-(9-(1-((6-chloro-2-(2-methyl-2H-tetrazol-5-yl)pyridin-3-yl)amino)ethyl)-7-methyl-4-(methyl-d3)-5-oxo-4,5-dihydro-3H-pyrazolo[3,4-c]isoquinolin-3-yl) pyrrolidin-1-yl)propanenitrile;
9-(1-((6-chloro-2-(1-methyl-1H-pyrazol-4-yl)pyridin-3-yl)amino)ethyl)-3-(1-(2-hydroxyacetyl) pyrrolidin-3-yl)-7-methyl-4-(methyl-d3)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-(1-(dimethylglycyl) pyrrolidin-3-yl)-7-methyl-4-(methyl-d3)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-7-methyl-4-(methyl-d3)-3-(1-(1-methyl-1H-pyrazole-5-carbonyl) pyrrolidin-3-yl)-3,4-dihydro-5H-pyrazolo[3,4-c]isoquinolin-5-one;
1-(3-(9-(1-((6-chloro-2-(1-methyl-1H-pyrazol-4-yl)pyridin-3-yl)amino)ethyl)-7-methyl-4-(methyl-d3)-5-oxo-4,5-dihydro-3H-pyrazolo[3,4-c]isoquinolin-3-yl) pyrrolidine-1-carbonyl)cyclopropane-1-carbonitrile;
9-(1-((6-chloro-2-(1-methyl-1H-pyrazol-4-yl)pyridin-3-yl)amino)ethyl)-4,7-dimethyl-3-(1-methylpiperidin-4-yl)-3,4-dihydro-5H-pyrazolo[3,4-c]isoquinolin-5-one;
9-[1-[[6-chloro-2-(1-methyl-1,2,4-triazol-3-yl)-3-pyridyl]amino]ethyl]-4,7-dimethyl-3-(1-methyl-4-piperidyl)pyrazolo[3,4-c]isoquinolin-5-one;
9-[1-[[6-chloro-2-(1-ethylpyrazol-4-yl)-3-pyridyl]amino]ethyl]-4,7-dimethyl-3-(1-methyl-4-piperidyl)pyrazolo[3,4-c]isoquinolin-5-one;
9-[1-[[6-chloro-2-(2-methyltetrazol-5-yl)-3-pyridyl]amino]ethyl]-4,7-dimethyl-3-(1-methyl-4-piperidyl)pyrazolo[3,4-c]isoquinolin-5-one;
9-[1-[[6-chloro-2-(1,3,4-oxadiazol-2-yl)-3-pyridyl]amino]ethyl]-4,7-dimethyl-3-(1-methyl-4-piperidyl)pyrazolo[3,4-c]isoquinolin-5-one;
5-[6-chloro-3-[1-[4,7-dimethyl-3-(1-methyl-4-piperidyl)-5-oxo-pyrazolo[3,4-c]isoquinolin-9-yl]ethylamino]-2-pyridyl]-N-methyl-pyridine-2-carboxamide;
3-[6-chloro-3-[1-[4,7-dimethyl-3-(1-methyl-4-piperidyl)-5-oxo-pyrazolo[3,4-c]isoquinolin-9-yl]ethylamino]-2-pyridyl]-4H-1,2,4-oxadiazol-5-one;
9-[1-[[6-chloro-2-(1,3-dimethylpyrazol-4-yl)-3-pyridyl]amino]ethyl]-4,7-dimethyl-3-(1-methyl-4-piperidyl)pyrazolo[3,4-c]isoquinolin-5-one;
9-[1-[[6-chloro-2-(1-methyl-6-oxo-3-pyridyl)-3-pyridyl]amino]ethyl]-4,7-dimethyl-3-(1-methyl-4-piperidyl)pyrazolo[3,4-c]isoquinolin-5-one;
9-[1-[[6-chloro-2-(4-hydroxy-1-piperidyl)-3-pyridyl]amino]ethyl]-4,7-dimethyl-3-(1-methyl-4-piperidyl)pyrazolo[3,4-c]isoquinolin-5-one;
9-[1-[[6-chloro-2-(3-fluoro-4-pyridyl)-3-pyridyl]amino]ethyl]-4,7-dimethyl-3-(1-methyl-4-piperidyl)pyrazolo[3,4-c]isoquinolin-5-one;
9-[1-[[6-chloro-2-(3-pyridyl)-3-pyridyl]amino]ethyl]-4,7-dimethyl-3-(1-methyl-4-piperidyl)pyrazolo[3,4-c]isoquinolin-5-one;
5-[3-[1-[4,7-dimethyl-3-(1-methyl-4-piperidyl)-5-oxo-pyrazolo[3,4-c]isoquinolin-9-yl]ethylamino]-6-methyl-2-pyridyl]-N-methyl-pyridine-2-carboxamide;
9-[1-[[6-chloro-2-(1-methyl-2-oxo-4-pyridyl)-3-pyridyl]amino]ethyl]-4,7-dimethyl-3-(1-methyl-4-piperidyl)pyrazolo[3,4-c]isoquinolin-5-one;
9-[1-[[6-chloro-2-(1-methylpyrazol-4-yl)-3-pyridyl]amino]ethyl]-3-[1-(2-hydroxy-2-methyl-propyl)-4-piperidyl]-4,7-dimethyl-pyrazolo[3,4-c]isoquinolin-5-one;
9-[1-[[6-chloro-2-(1-methylpyrazol-4-yl)-3-pyridyl]amino]ethyl]-3-[1-[(2R)-2-hydroxypropyl]-4-piperidyl]-4,7-dimethyl-pyrazolo[3,4-c]isoquinolin-5-one;
9-[1-[[6-chloro-2-(1-methylpyrazol-4-yl)-3-pyridyl]amino]ethyl]-3-[1-[(2S)-2-hydroxypropyl]-4-piperidyl]-4,7-dimethyl-pyrazolo[3,4-c]isoquinolin-5-one;
9-(1-((6-chloro-2-(1-methyl-1H-1,2,4-triazol-3-yl)pyridin-3-yl)amino)ethyl)-3-(1-(2-hydroxy-2-methylpropyl)piperidin-4-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(piperidin-4-yl)-3,4-dihydro-5H-pyrazolo[3,4-c]isoquinolin-5-one;
9-[1-[[6-chloro-2-(1-methyl-1,2,4-triazol-3-yl)-3-pyridyl]amino]ethyl]-3-[1-(2-hydroxyethyl)-4-piperidyl]-4,7-dimethyl-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-((1-methyl-1H-imidazol-2-yl)methyl)piperidin-4-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-(oxetan-3-yl)piperidin-4-yl)-3,4-dihydro-5H-pyrazolo[3,4-c]isoquinolin-5-one;
3-[4-[9-[1-[[6-chloro-2-(1-methyl-1,2,4-triazol-3-yl)-3-pyridyl]amino]ethyl]-4,7-dimethyl-5-oxo-pyrazolo[3,4-c]isoquinolin-3-yl]-1-piperidyl]propanenitrile;
9-(1-((6-chloro-2-(1-methyl-1H-1,2,4-triazol-3-yl)pyridin-3-yl)amino)ethyl)-3-(1-(2,2-difluoroethyl)piperidin-4-yl)-4,7-dimethyl-3,4-dihydro-5H-pyrazolo[3,4-c]isoquinolin-5-one;
3-(1-acetylpiperidin-4-yl)-9-(1-((6-chloro-2-(1-methyl-1H-pyrazol-4-yl)pyridin-3-yl)amino)ethyl)-4,7-dimethyl-3,4-dihydro-5H-pyrazolo[3,4-c]isoquinolin-5-one;
9-[1-[[6-chloro-2-(1-methylpyrazol-4-yl)-3-pyridyl]amino]ethyl]-4,7-dimethyl-3-(1-methylsulfonyl-4-piperidyl)pyrazolo[3,4-c]isoquinolin-5-one;
methyl 4-[9-[1-[[6-chloro-2-(1-methylpyrazol-4-yl)-3-pyridyl]amino]ethyl]-4,7-dimethyl-5-oxo-pyrazolo[3,4-c]isoquinolin-3-yl]piperidine-1-carboxylate;
9-[1-[[6-chloro-2-(1-methylpyrazol-4-yl)-3-pyridyl]amino]ethyl]-3-(1-cyclopropylsulfonyl-4-piperidyl)-4,7-dimethyl-pyrazolo[3,4-c]isoquinolin-5-one;
4-(9-(1-((6-chloro-2-(1-methyl-1H-pyrazol-4-yl)pyridin-3-yl)amino)ethyl)-4,7-dimethyl-5-oxo-4,5-dihydro-3H-pyrazolo[3,4-c]isoquinolin-3-yl)-N,N-dimethylpiperidine-1-carboxamide;
3-(1-acetylpiperidin-4-yl)-9-(1-((6-chloro-1′-methyl-6′-oxo-1′,6′-dihydro-[2,3′-bipyridin]-3-yl)amino)ethyl)-4,7-dimethyl-3,4-dihydro-5H-pyrazolo[3,4-c]isoquinolin-5-one;
3-(1-acetylpiperidin-4-yl)-9-(1-((6-chloro-2-(1-methyl-1H-1,2,4-triazol-3-yl)pyridin-3-yl)amino)ethyl)-4,7-dimethyl-3,4-dihydro-5H-pyrazolo[3,4-c]isoquinolin-5-one;
4,7-dimethyl-9-(1-((6-methyl-2-(1-methyl-1H-pyrazol-4-yl)pyridin-3-yl)amino)ethyl)-3-(1-(methylsulfonyl)piperidin-4-yl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-(methylsulfonyl)piperidin-4-yl)-3,4-dihydro-5H-pyrazolo[3,4-c]isoquinolin-5-one;
9-(1-((6-chloro-2-(2-methyl-1-oxoisoindolin-5-yl)pyridin-3-yl)amino)ethyl)-4,7-dimethyl-3-(1-(methylsulfonyl)piperidin-4-yl)-3,4-dihydro-5H-pyrazolo[3,4-c]isoquinolin-5-one;
9-(1-((6-chloro-2-(quinolin-6-yl)pyridin-3-yl)amino)ethyl)-4,7-dimethyl-3-(1-(methylsulfonyl)piperidin-4-yl)-3,4-dihydro-5H-pyrazolo[3,4-c]isoquinolin-5-one;
9-[1-[[6-chloro-2-(1-methylpyrazol-4-yl)-3-pyridyl]amino]ethyl]-3-[1-(2-hydroxyacetyl)-4-piperidyl]-4,7-dimethyl-pyrazolo[3,4-c]isoquinolin-5-one;
1-(4-(9-(1-((6-chloro-2-(1-methyl-1H-1,2,4-triazol-3-yl)pyridin-3-yl)amino)ethyl)-4,7-dimethyl-5-oxo-4,5-dihydro-3H-pyrazolo[3,4-c]isoquinolin-3-yl)piperidine-1-carbonyl)cyclopropane-1-carbonitrile;
9-(1-((6-chloro-2-(1-methyl-1H-1,2,4-triazol-3-yl)pyridin-3-yl)amino)ethyl)-4,7-dimethyl-3-(1-(1-methyl-1H-pyrazole-5-carbonyl)piperidin-4-yl)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-3-(1-(dimethylglycyl)piperidin-4-yl)-4,7-dimethyl-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-3-(1-(methyl-L-prolyl)piperidin-4-yl)-3,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-4-methyl-3-(1-methylpiperidin-4-yl)-5-oxo-4,5-dihydro-3H-pyrazolo[3,4-c]isoquinoline-7-carbonitrile;
9-(1-((6-chloro-2-(1-methyl-1H-1,2,4-triazol-3-yl)pyridin-3-yl)amino)ethyl)-4-ethyl-7-methyl-2-(tetrahydro-2H-pyran-4-yl)-2,4-dihydro-5H-pyrazolo[3,4-c]isoquinolin-5-one;
3-(6-chloro-3-((1-(4,7-dimethyl-5-oxo-2-(tetrahydro-2H-pyran-4-yl)-4,5-dihydro-2H-pyrazolo[3,4-c]isoquinolin-9-yl)ethyl)amino)pyridin-2-yl)-1,2,4-oxadiazol-5(4H)-one;
9-(1-((6-chloro-2-(1-methyl-1H-1,2,4-triazol-3-yl)pyridin-3-yl)amino)ethyl)-4-ethyl-7-methyl-2-(1-methylpiperidin-4-yl)-2,4-dihydro-5H-pyrazolo[3,4-c]isoquinolin-5-one;
2-(1-acetylpiperidin-4-yl)-9-(1-((6-chloro-2-(1-methyl-1H-1,2,4-triazol-3-yl)pyridin-3-yl)amino)ethyl)-4-ethyl-7-methyl-2,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4-ethyl-2-(1-(2-hydroxyethyl)piperidin-4-yl)-7-methyl-2,4-dihydro-5H-pyrazolo[3,4-c]isoquinolin-5-one;
3-(4-(9-(1-((6-chloro-2-(1-methyl-1H-1,2,4-triazol-3-yl)pyridin-3-yl)amino)ethyl)-4-ethyl-7-methyl-5-oxo-4,5-dihydro-2H-pyrazolo[3,4-c]isoquinolin-2-yl)piperidin-1-yl)propanenitrile;
9-(1-((6-chloro-2-(1-methyl-1H-1,2,4-triazol-3-yl)pyridin-3-yl)amino)ethyl)-4-ethyl-7-methyl-2-(1-methylazetidin-3-yl)-2,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-4,7-dimethyl-2-(1-methylpiperidin-4-yl)-2,4-dihydro-5H-pyrazolo[3,4-c]isoquinolin-5-one;
9-(1-((6-chloro-2-(1-methyl-1H-pyrazol-4-yl)pyridin-3-yl)amino)ethyl)-2-(1-isopropylpiperidin-4-yl)-4,7-dimethyl-2,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4,7-dimethyl-2-(1-methylpiperidin-4-yl)-2,4-dihydro-5H-pyrazolo[3,4-c]isoquinolin-5-one;
2-(1-acetylpiperidin-4-yl)-9-(1-((6-chloro-2-(1-methyl-1H-pyrazol-4-yl)pyridin-3-yl)amino)ethyl)-4,7-dimethyl-2,4-dihydro-5H-pyrazolo[3,4-c]isoquinolin-5-one;
ethyl 4-(9-(1-((6-chloro-2-(1-methyl-1H-pyrazol-4-yl)pyridin-3-yl)amino)ethyl)-4,7-dimethyl-5-oxo-4,5-dihydro-2H-pyrazolo[3,4-c]isoquinolin-2-yl)piperidine-1-carboxylate;
4-(9-(1-((6-chloro-2-(1-methyl-1H-pyrazol-4-yl)pyridin-3-yl)amino)ethyl)-4,7-dimethyl-5-oxo-4,5-dihydro-2H-pyrazolo[3,4-c]isoquinolin-2-yl)-N,N-dimethylpiperidine-1-carboxamide;
9-(1-((6-chloro-2-(1-methyl-1H-pyrazol-4-yl)pyridin-3-yl)amino)ethyl)-2-(1-(2,2-difluoroethyl)piperidin-4-yl)-4,7-dimethyl-2,4-dihydro-5H-pyrazolo[3,4-c]isoquinolin-5-one;
3-[6-chloro-3-[1-[7-methyl-5-oxo-3-[(3S)-tetrahydrofuran-3-yl]-4-(trideuteriomethyl)pyrazolo[3,4-c]isoquinolin-9-yl]ethylamino]-2-pyridyl]-4H-1,2,4-oxadiazol-5-one;
9-(1-((6-chloro-2-(1-methyl-1H-pyrazol-4-yl)pyridin-3-yl)amino)ethyl)-7-methyl-4-(methyl-d3)-3-((S)-tetrahydrofuran-3-yl)-3,4-dihydro-5H-pyrazolo[3,4-c]isoquinolin-5-one;
3-(6-chloro-3-((1-(4-ethyl-7-methyl-5-oxo-3-((S)-tetrahydrofuran-3-yl)-4,5-dihydro-3H-pyrazolo[3,4-c]isoquinolin-9-yl)ethyl)amino)pyridin-2-yl)-1,2,4-oxadiazol-5(4H)-one;
9-(1-((6-chloro-2-(1-methyl-1H-1,2,4-triazol-3-yl)pyridin-3-yl)amino)ethyl)-4-ethyl-7-methyl-3-((S)-tetrahydrofuran-3-yl)-3,4-dihydro-5H-pyrazolo[3,4-c]isoquinolin-5-one;
9-(1-((6-chloro-2-(1-(1-methylpiperidin-4-yl)-1H-pyrazol-4-yl)pyridin-3-yl)amino)ethyl)-4-ethyl-7-methyl-3-((S)-tetrahydrofuran-3-yl)-3,4-dihydro-5H-pyrazolo[3,4-c]isoquinolin-5-one;
9-(1-((6-chloro-2′-methyl-[2,4′-bipyridin]-3-yl)amino)ethyl)-4-ethyl-7-methyl-3-((S)-tetrahydrofuran-3-yl)-3,4-dihydro-5H-pyrazolo[3,4-c]isoquinolin-5-one;
9-(1-((6-chloro-1′-methyl-6′-oxo-1′,6′-dihydro-[2,3′-bipyridin]-3-yl)amino)ethyl)-4-ethyl-7-methyl-3-((S)-tetrahydrofuran-3-yl)-3,4-dihydro-5H-pyrazolo[3,4-c]isoquinolin-5-one;
6-chloro-3-((1-(4-ethyl-7-methyl-5-oxo-3-((S)-tetrahydrofuran-3-yl)-4,5-dihydro-3H-pyrazolo[3,4-c]isoquinolin-9-yl)ethyl)amino)-N-methyl-[2,3′-bipyridine]-6′-carboxamide;
9-(1-((6-chloro-2-(1-methyl-1H-1,2,4-triazol-3-yl)pyridin-3-yl)amino)ethyl)-4-cyclopropyl-3-ethyl-7-methyl-3,4-dihydro-5H-pyrazolo[3,4-c]isoquinolin-5-one;
9-[1-[[6-chloro-2-(1-methyl-6-oxo-3-pyridyl)-3-pyridyl]amino]ethyl]-4-cyclopropyl-3-ethyl-7-methyl-pyrazolo[3,4-c]isoquinolin-5-one;
9-[1-[[6-chloro-2-(1-methyl-1,2,4-triazol-3-yl)-3-pyridyl]amino]ethyl]-3-ethyl-7-methyl-4-tetrahydrofuran-3-yl-pyrazolo[3,4-c]isoquinolin-5-one;
9-[1-[[6-chloro-2-(1-methyl-1,2,4-triazol-3-yl)-3-pyridyl]amino]ethyl]-3-ethyl-4-(2-hydroxyethyl)-7-methyl-pyrazolo[3,4-c]isoquinolin-5-one;
4-(6-chloro-3-(((3-ethyl-4,7-dimethyl-5-oxo-4,5-dihydro-3H-pyrazolo[3,4-c]isoquinolin-9-yl)methyl)amino)pyridin-2-yl)-2-fluoro-N-methylbenzamide;
9-(1-((6-chloro-2-(3-methyl-1H-pyrazol-4-yl)pyridin-3-yl)oxy)ethyl)-3-ethyl-4,7-dimethyl-3,4-dihydro-5H-pyrazolo[3,4-c]isoquinolin-5-one;
3-(6-chloro-3-((1-(3-ethyl-7-methyl-4-(methyl-d3)-5-oxo-4,5-dihydro-3H-pyrazolo[3,4-c]isoquinolin-9-yl)ethyl)amino)pyridin-2-yl)-1,2,4-oxadiazol-5(4H)-one;
9-(1-((6-chloro-2-(1-methyl-1H-1,2,4-triazol-3-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(4-methyl-4H-1,2,4-triazol-3-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(1H-tetrazol-5-yl)pyridin-3-yl)amino)ethyl)-3-ethyl-4,7-dimethyl-3,4-dihydro-5H-pyrazolo[3,4-c]isoquinolin-5-one;
9-(1-((6-chloro-2-(1-methyl-1H-1,2,3-triazol-5-yl)pyridin-3-yl)amino)ethyl)-3-ethyl-4,7-dimethyl-3,4-dihydro-5H-pyrazolo[3,4-c]isoquinolin-5-one;
4-(6-chloro-3-((1-(3-ethyl-4,7-dimethyl-5-oxo-4,5-dihydro-3H-pyrazolo[3,4-c]isoquinolin-9-yl)ethyl)amino)pyridin-2-yl)benzoic acid;
3-ethyl-4,7-dimethyl-9-(1-((6-methyl-2-(3-methyl-1H-pyrazol-4-yl)pyridin-3-yl)amino)ethyl)-3,4-dihydro-5H-pyrazolo[3,4-c]isoquinolin-5-one;
9-(1-((6-chloro-2-(1-methyl-1H-1,2,4-triazol-3-yl)pyridin-3-yl)amino)ethyl)-4-(2,2-difluoroethyl)-3-ethyl-7-methyl-3,4-dihydro-5H-pyrazolo[3,4-c]isoquinolin-5-one;
9-(1-((6-chloro-2′-methyl-[2,4′-bipyridin]-3-yl)amino)ethyl)-4-(2,2-difluoroethyl)-3-ethyl-7-methyl-3,4-dihydro-5H-pyrazolo[3,4-c]isoquinolin-5-one;
9-(1-((6-chloro-2-(pyrimidin-5-yl)pyridin-3-yl)amino)ethyl)-4-(2,2-difluoroethyl)-3-ethyl-7-methyl-3,4-dihydro-5H-pyrazolo[3,4-c]isoquinolin-5-one;
9-(1-((6-chloro-1′-methyl-6′-oxo-1′,6′-dihydro-[2,3′-bipyridin]-3-yl)amino)ethyl)-4-(2,2-difluoroethyl)-3-ethyl-7-methyl-3,4-dihydro-5H-pyrazolo[3,4-c]isoquinolin-5-one;
6-chloro-3-((1-(4-(2,2-difluoroethyl)-3-ethyl-7-methyl-5-oxo-4,5-dihydro-3H-pyrazolo[3,4-c]isoquinolin-9-yl)ethyl)amino)-N-methyl-[2,3′-bipyridine]-6′-carboxamide;
9-[1-[[2-(1-acetyl-4-piperidyl)-6-chloro-3-pyridyl]amino]ethyl]-3-ethyl-4,7-dimethyl-pyrazolo[3,4-c]isoquinolin-5-one;
9-(1-((6-chloro-2-(1-methyl-1H-1,2,4-triazol-3-yl)pyridin-3-yl)amino)ethyl)-3-(2-hydroxyethyl)-4,7-dimethyl-3,4-dihydro-5H-pyrazolo[3,4-c]isoquinolin-5-one;
4-(6-chloro-3-((1-(3-(2-hydroxyethyl)-4,7-dimethyl-5-oxo-4,5-dihydro-3H-pyrazolo[3,4-c]isoquinolin-9-yl)ethyl)amino)pyridin-2-yl)-2-fluoro-N-methylbenzamide;
9-(1-((6-chloro-2-(1-methyl-1H-pyrazol-4-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(4-hydroxypiperidin-1-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(quinolin-6-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(1-methyl-1H-indazol-5-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(2-methyl-1-oxoisoindolin-5-yl)pyridin-3-yl)amino)ethyl)-3-(2-hydroxyethyl)-4,7-dimethyl-3,4-dihydro-5H-pyrazolo[3,4-c]isoquinolin-5-one;
3-(2-hydroxyethyl)-4,7-dimethyl-9-(1-((6-methyl-2-(1-methyl-1H-pyrazol-4-yl)pyridin-3-yl)amino)ethyl)-3,4-dihydro-5H-pyrazolo[3,4-c]isoquinolin-5-one;
9-(1-((6-chloro-2-(1,3,4-oxadiazol-2-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(2-methyl-2H-tetrazol-5-yl)pyridin-3-yl)amino)ethyl)-3-(2-hydroxyethyl)-4,7-dimethyl-3,4-dihydro-5H-pyrazolo[3,4-c]isoquinolin-5-one;
9-(1-((6-chloro-2-(pyrimidin-5-yl)pyridin-3-yl)amino)ethyl)-3-(2-hydroxyethyl)-4,7-dimethyl-3,4-dihydro-5H-pyrazolo[3,4-c]isoquinolin-5-one;
5-[6-chloro-3-[1-[3-(2-hydroxyethyl)-4,7-dimethyl-5-oxo-pyrazolo[3,4-c]isoquinolin-9-yl]ethylamino]-2-pyridyl]-N-methyl-pyridine-2-carboxamide;
9-[1-[[6-chloro-2-(1-methyl-6-oxo-3-pyridyl)-3-pyridyl]amino]ethyl]-3-(2-hydroxyethyl)-4,7-dimethyl-pyrazolo[3,4-c]isoquinolin-5-one;
9-[1-[[6-chloro-2-(1-methyl-2-oxo-4-pyridyl)-3-pyridyl]amino]ethyl]-3-(2-hydroxyethyl)-4,7-dimethyl-pyrazolo[3,4-c]isoquinolin-5-one;
9-[1-[[6-chloro-2-(2-methoxy-4-pyridyl)-3-pyridyl]amino]ethyl]-3-(2-hydroxyethyl)-4,7-dimethyl-pyrazolo[3,4-c]isoquinolin-5-one;
9-[1-[[6-chloro-2-(1-methyl-4-piperidyl)-3-pyridyl]amino]ethyl]-3-(2-hydroxyethyl)-4,7-dimethyl-pyrazolo[3,4-c]isoquinolin-5-one;
9-[1-[[2-(1-acetyl-4-piperidyl)-6-chloro-3-pyridyl]amino]ethyl]-3-(2-hydroxyethyl)-4,7-dimethyl-pyrazolo[3,4-c]isoquinolin-5-one;
9-[1-[[6-chloro-2-(1-methyl-1,2,4-triazol-3-yl)-3-pyridyl]amino]ethyl]-4-(2,2-difluoroethyl)-3-(2-hydroxyethyl)-7-methyl-pyrazolo[3,4-c]isoquinolin-5-one;
5-[6-chloro-3-[1-[4-(2,2-difluoroethyl)-3-(2-hydroxyethyl)-7-methyl-5-oxo-pyrazolo[3,4-c]isoquinolin-9-yl]ethylamino]-2-pyridyl]-N-methyl-pyridine-2-carboxamide;
9-[1-[[6-chloro-2-(1-methyl-1,2,4-triazol-3-yl)-3-pyridyl]amino]ethyl]-4-ethyl-3-(2-hydroxyethyl)-7-methyl-pyrazolo[3,4-c]isoquinolin-5-one;
9-[1-[(6-chloro-2-morpholino-3-pyridyl)amino]ethyl]-4-ethyl-3-(2-hydroxyethyl)-7-methyl-pyrazolo[3,4-c]isoquinolin-5-one;
9-[1-[[6-chloro-2-(1-methylpyrazol-4-yl)-3-pyridyl]amino]ethyl]-4,7-dimethyl-3-(2-morpholinoethyl)pyrazolo[3,4-c]isoquinolin-5-one;
9-(1-((6-chloro-3′-fluoro-[2,4′-bipyridin]-3-yl)amino)ethyl)-4,7-dimethyl-3-(2-morpholinoethyl)-3,4-dihydro-5H-pyrazolo[3,4-c]isoquinolin-5-one;
9-(1-((6-chloro-3′-fluoro-[2,4′-bipyridin]-3-yl)amino)ethyl)-4,7-dimethyl-3-(2-(4-methylpiperazin-1-yl)ethyl)-3,4-dihydro-5H-pyrazolo[3,4-c]isoquinolin-5-one;
4-(6-chloro-3-((1-(4,7-dimethyl-3-(2-morpholinoethyl)-5-oxo-4,5-dihydro-3H-pyrazolo[3,4-c]isoquinolin-9-yl)ethyl)amino)pyridin-2-yl)-2-fluoro-N-methylbenzamide;
4-(6-chloro-3-((1-(4,7-dimethyl-3-(2-(4-methylpiperazin-1-yl)ethyl)-5-oxo-4,5-dihydro-3H-pyrazolo[3,4-c]isoquinolin-9-yl)ethyl)amino)pyridin-2-yl)-2-fluoro-N-methylbenzamide;
9-(1-((6-chloro-2-(1-methyl-1H-1,2,4-triazol-3-yl)pyridin-3-yl)amino)ethyl)-4-(2,2-difluoroethyl)-3-(2-(5,6-dihydro-[1,2,4]triazolo[1,5-a]pyrazin-7(8H)-yl)ethyl)-7-methyl-3,4-dihydro-5H-pyrazolo[3,4-c]isoquinolin-5-one;
9-[1-[[6-chloro-2-(1-methyl-1,2,4-triazol-3-yl)-3-pyridyl]amino]ethyl]-4-ethyl-7-methyl-3-(2-pyridyl)pyrazolo[3,4-c]isoquinolin-5-one;
9-(1-((6-chloro-2-morpholinopyridin-3-yl)amino)ethyl)-4-ethyl-7-methyl-3-(pyridin-2-yl)-3,4-dihydro-5H-pyrazolo[3,4-c]isoquinolin-5-one;
6-chloro-3-((1-(4-ethyl-7-methyl-5-oxo-3-(pyridin-2-yl)-4,5-dihydro-3H-pyrazolo[3,4-c]isoquinolin-9-yl)ethyl)amino)-N-methyl-[2,3′-bipyridine]-6′-carboxamide;
3-(6-chloro-3-((1-(4,7-dimethyl-5-oxo-3-(pyridin-2-yl)-4,5-dihydro-3H-pyrazolo[3,4-c]isoquinolin-9-yl)ethyl)amino)pyridin-2-yl)-1,2,4-oxadiazol-5(4H)-one;
9-(1-((6-chloro-2-(1-methyl-1H-pyrazol-4-yl)pyridin-3-yl)amino)ethyl)-4,7-dimethyl-3-(pyridin-2-yl)-3,4-dihydro-5H-pyrazolo[3,4-c]isoquinolin-5-one;
9-[1-[[6-chloro-2-(1-methyl-1,2,4-triazol-3-yl)-3-pyridyl]amino]ethyl]-4-ethyl-7-methyl-3-(3-pyridyl)pyrazolo[3,4-c]isoquinolin-5-one;
9-[1-[[6-chloro-2-(1-methylpyrazol-4-yl)-3-pyridyl]amino]ethyl]-4,7-dimethyl-3-(2-pyridylmethyl)pyrazolo[3,4-c]isoquinolin-5-one;
3-(6-chloro-3-((1-(4,7-dimethyl-5-oxo-3-(pyridin-2-ylmethyl)-4,5-dihydro-3H-pyrazolo[3,4-c]isoquinolin-9-yl)ethyl)amino)pyridin-2-yl)-1,2,4-oxadiazol-5(4H)-one;
9-[1-[[6-chloro-2-(1-methyl-1,2,4-triazol-3-yl)-3-pyridyl]amino]ethyl]-4,7-dimethyl-3-(1-methyl-3-piperidyl)pyrazolo[3,4-c]isoquinolin-5-one;
9-[1-[[6-chloro-2-(1-methyl-1,2,4-triazol-3-yl)-3-pyridyl]amino]ethyl]-3-[1-(2-hydroxyethyl)-3-piperidyl]-4,7-dimethyl-pyrazolo[3,4-c]isoquinolin-5-one;
9-[1-[[6-chloro-2-(1-methylpyrazol-4-yl)-3-pyridyl]amino]ethyl]-4,7-dimethyl-3-(1-methyl-3-piperidyl)pyrazolo[3,4-c]isoquinolin-5-one;
9-[1-[[6-chloro-2-(1-methylpyrazol-4-yl)-3-pyridyl]amino]ethyl]-3-[1-(2-hydroxyethyl)-3-piperidyl]-4,7-dimethyl-pyrazolo[3,4-c]isoquinolin-5-one;
methyl 3-[9-[1-[[6-chloro-2-(1-methylpyrazol-4-yl)-3-pyridyl]amino]ethyl]-4,7-dimethyl-5-oxo-pyrazolo[3,4-c]isoquinolin-3-yl]piperidine-1-carboxylate;
5-[6-chloro-3-[1-[4,7-dimethyl-3-(1-methyl-4-piperidyl)-5-oxo-pyrazolo[3,4-c]isoquinolin-9-yl]ethoxy]-2-pyridyl]-N-methyl-pyridine-2-carboxamide;
5-[3-[1-[3-(1-acetyl-4-piperidyl)-4,7-dimethyl-5-oxo-pyrazolo[3,4-c]isoquinolin-9-yl]ethoxy]-6-chloro-2-pyridyl]-N-methyl-pyridine-2-carboxamide;
10-(1-((6-chloro-2-(1-methyl-1H-1,2,4-triazol-3-yl)pyridin-3-yl)amino)ethyl)-3-(hydroxymethyl)-8-methyl-4,5-dihydro-3H,6H-2,2a,5a-triazaaceanthrylen-6-one;
10-(1-((6-chloro-2-(2-methyl-2H-tetrazol-5-yl)pyridin-3-yl)amino)ethyl)-3-(hydroxymethyl)-8-methyl-4,5-dihydro-3H,6H-2,2a,5a-triazaaceanthrylen-6-one;
10-(1-((6-chloro-2-(1,3,4-oxadiazol-2-yl)pyridin-3-yl)amino)ethyl)-3-(hydroxymethyl)-8-methyl-4,5-dihydro-3H,6H-2,2a,5a-triazaaceanthrylen-6-one;
10-(1-((6-chloro-2-(1-methyl-1H-pyrazol-4-yl)pyridin-3-yl)amino)ethyl)-3-(hydroxymethyl)-8-methyl-4,5-dihydro-3H,6H-2,2a,5a-triazaaceanthrylen-6-one;
10-(1-((6-chloro-2-(3-methyl-1H-pyrazol-4-yl)pyridin-3-yl)amino)ethyl)-3-(hydroxymethyl)-8-methyl-4,5-dihydro-3H,6H-2,2a,5a-triazaaceanthrylen-6-one;
10-(1-((6-chloro-1′-methyl-6′-oxo-1′,6′-dihydro-[2,3′-bipyridin]-3-yl)amino)ethyl)-3-(hydroxymethyl)-8-methyl-4,5-dihydro-3H,6H-2,2a,5a-triazaaceanthrylen-6-one;
10-(1-((6-chloro-2-(1-methyl-1H-1,2,4-triazol-3-yl)pyridin-3-yl)amino)ethyl)-3-((dimethylamino)methyl)-8-methyl-4,5-dihydro-3H,6H-2,2a,5a-triazaaceanthrylen-6-one;
10-(1-((6-chloro-2-(4-hydroxypiperidin-1-yl)pyridin-3-yl)amino)ethyl)-8-methyl-4,5-dihydro-3H,6H-2,2a,5a-triazaaceanthrylen-6-one;
1-(6-chloro-3-((1-(8-methyl-6-oxo-4,5-dihydro-3H,6H-2,2a,5a-triazaaceanthrylen-10-yl)ethyl)amino)pyridin-2-yl)-N-methylpiperidine-4-carboxamide;
10-(1-((2-(4-acetylpiperazin-1-yl)-6-chloropyridin-3-yl)amino)ethyl)-8-methyl-4,5-dihydro-3H,6H-2,2a,5a-triazaaceanthrylen-6-one;
10-(1-((6-chloro-2-(1,3,4-oxadiazol-2-yl)pyridin-3-yl)amino)ethyl)-8-methyl-4,5-dihydro-3H,6H-2,2a,5a-triazaaceanthrylen-6-one;
3-(6-chloro-3-((1-(8-methyl-6-oxo-4,5-dihydro-3H,6H-2,2a,5a-triazaaceanthrylen-10-yl)ethyl)amino)pyridin-2-yl)-1,2,4-oxadiazol-5(4H)-one;
10-(1-((6-chloro-2-(1-methyl-1H-1,2,4-triazol-3-yl)pyridin-3-yl)amino)ethyl)-8-methyl-4,5-dihydro-3H,6H-2,2a,5a-triazaaceanthrylen-6-one;
10-(1-((6-chloro-2-(1-(2-hydroxyethyl)-1H-1,2,4-triazol-3-yl)pyridin-3-yl)amino)ethyl)-8-methyl-4,5-dihydro-3H,6H-2,2a,5a-triazaaceanthrylen-6-one;
10-(1-((6-chloro-2-(2-methyl-2H-tetrazol-5-yl)pyridin-3-yl)amino)ethyl)-8-methyl-4,5-dihydro-3H,6H-2,2a,5a-triazaaceanthrylen-6-one;
10-(1-((6-chloro-2-(1-(tetrahydro-2H-pyran-4-yl)-1H-pyrazol-4-yl)pyridin-3-yl)amino)ethyl)-8-methyl-4,5-dihydro-3H,6H-2,2a,5a-triazaaceanthrylen-6-one;
10-(1-((6-chloro-2-(1-(2-hydroxyethyl)-1H-pyrazol-4-yl)pyridin-3-yl)amino)ethyl)-8-methyl-4,5-dihydro-3H,6H-2,2a,5a-triazaaceanthrylen-6-one;
10-(1-((6-chloro-2′-methyl-[2,4′-bipyridin]-3-yl)amino)ethyl)-8-methyl-4,5-dihydro-3H,6H-2,2a,5a-triazaaceanthrylen-6-one;
10-(1-((6-chloro-[2,3′-bipyridin]-3-yl)amino)ethyl)-8-methyl-4,5-dihydro-3H,6H-2,2a,5a-triazaaceanthrylen-6-one;
10-(1-((6-chloro-1′-methyl-6′-oxo-1′,6′-dihydro-[2,3′-bipyridin]-3-yl)amino)ethyl)-8-methyl-4,5-dihydro-3H,6H-2,2a,5a-triazaaceanthrylen-6-one;
4-(6-chloro-3-((1-(8-methyl-6-oxo-4,5-dihydro-3H,6H-2,2a,5a-triazaaceanthrylen-10-yl)ethyl)amino)pyridin-2-yl)-2-fluoro-N-methylbenzamide;
6-chloro-N-methyl-3-((1-(8-methyl-6-oxo-4,5-dihydro-3H,6H-2,2a,5a-triazaaceanthrylen-10-yl)ethyl)amino)-[2,3′-bipyridine]-6′-carboxamide;
10-(1-((6-chloro-6′-methoxy-[2,3′-bipyridin]-3-yl)amino)ethyl)-8-methyl-4,5-dihydro-3H,6H-2,2a,5a-triazaaceanthrylen-6-one;
10-(1-((6-chloro-2′-methoxy-[2,4′-bipyridin]-3-yl)amino)ethyl)-8-methyl-4,5-dihydro-3H,6H-2,2a,5a-triazaaceanthrylen-6-one;
10-(1-((6-chloro-2′-(dimethylamino)-[2,4′-bipyridin]-3-yl)amino)ethyl)-8-methyl-4,5-dihydro-3H,6H-2,2a,5a-triazaaceanthrylen-6-one;
10-(1-((6-chloro-2-(2-methyl-1-oxoisoindolin-5-yl)pyridin-3-yl)amino)ethyl)-8-methyl-4,5-dihydro-3H,6H-2,2a,5a-triazaaceanthrylen-6-one;
10-(1-((6-chloro-2-(quinolin-6-yl)pyridin-3-yl)amino)ethyl)-8-methyl-4,5-dihydro-3H,6H-2,2a,5a-triazaaceanthrylen-6-one;
10-(1-((6-chloro-2-(1-methyl-1H-indazol-5-yl)pyridin-3-yl)amino)ethyl)-8-methyl-4,5-dihydro-3H,6H-2,2a,5a-triazaaceanthrylen-6-one;
2-fluoro-N-methyl-4-(6-methyl-3-((1-(8-methyl-6-oxo-4,5-dihydro-3H,6H-2,2a,5a-triazaaceanthrylen-10-yl)ethyl)amino)pyridin-2-yl)benzamide;
8-methyl-10-(1-((6-methyl-2-(1-(tetrahydro-2H-pyran-4-yl)-1H-pyrazol-4-yl)pyridin-3-yl)amino)ethyl)-4,5-dihydro-3H,6H-2,2a,5a-triazaaceanthrylen-6-one;
10-(1-((2-(1-(2-hydroxyethyl)-1H-pyrazol-4-yl)-6-methylpyridin-3-yl)amino)ethyl)-8-methyl-4,5-dihydro-3H,6H-2,2a,5a-triazaaceanthrylen-6-one;
10-(1-((2′,6-dimethyl-[2,4′-bipyridin]-3-yl)amino)ethyl)-8-methyl-4,5-dihydro-3H,6H-2,2a,5a-triazaaceanthrylen-6-one;
10-(1-((2-(1-cyclopropyl-1H-pyrazol-4-yl)-6-methylpyridin-3-yl)amino)ethyl)-8-methyl-4,5-dihydro-3H,6H-2,2a,5a-triazaaceanthrylen-6-one;
7-(6-chloro-3-((1-(8-methyl-6-oxo-4,5-dihydro-3H,6H-2,2a,5a-triazaaceanthrylen-10-yl)ethyl)amino)pyridin-2-yl) hexahydro-3H-oxazolo[3,4-a]pyrazin-3-one;
10-(1-((6-chloro-2-(5-methyl-4,5,6,7-tetrahydropyrazolo[1,5-a]pyrazin-3-yl)pyridin-3-yl)amino)ethyl)-8-methyl-4,5-dihydro-3H,6H-2,2a,5a-triazaaceanthrylen-6-one;
10-(1-((6-chloro-6′-((dimethylamino)methyl)-[2,3′-bipyridin]-3-yl)amino)ethyl)-8-methyl-4,5-dihydro-3H,6H-2,2a,5a-triazaaceanthrylen-6-one;
3-(3-((1-(3,8-dimethyl-6-oxo-4,5-dihydro-3H,6H-2,2a,5a-triazaaceanthrylen-10-yl)ethyl)amino)-6-methylpyridin-2-yl)-1,2,4-oxadiazol-5(4H)-one;
8-methyl-10-(1-((6-methyl-2-(2-methyl-2H-tetrazol-5-yl)pyridin-3-yl)amino)ethyl)-4,5-dihydro-3H,6H-2,2a,5a-triazaaceanthrylen-6-one;
3-(3-((1-(3-ethyl-4,7-dimethyl-5-oxo-4,5-dihydro-3H-pyrazolo[3,4-c]isoquinolin-9-yl)ethyl)amino)-6-methylpyridin-2-yl)-1,2,4-oxadiazol-5(4H)-one;
10-(1-((6-chloro-2-(1-methyl-1H-1,2,4-triazol-3-yl)pyridin-3-yl)amino)-2-hydroxyethyl)-8-methyl-4,5-dihydro-3H,6H-2,2a,5a-triazaaceanthrylen-6-one;
10-(1-((6-chloro-2-(1-methyl-1H-pyrazol-4-yl)pyridin-3-yl)amino)-2-hydroxyethyl)-8-methyl-4,5-dihydro-3H,6H-2,2a,5a-triazaaceanthrylen-6-one;
10-(1-((6-chloro-2-(1,5-dimethyl-1H-pyrazol-4-yl)pyridin-3-yl)amino)-2-hydroxyethyl)-8-methyl-4,5-dihydro-3H,6H-2,2a,5a-triazaaceanthrylen-6-one;
10-(1-((6-chloro-2-(1,3,4-oxadiazol-2-yl)pyridin-3-yl)amino)-2-hydroxyethyl)-8-methyl-4,5-dihydro-3H,6H-2,2a,5a-triazaaceanthrylen-6-one;
10-(1-((6-chloro-2-(2-methyl-2H-tetrazol-5-yl)pyridin-3-yl)amino)-2-hydroxyethyl)-8-methyl-4,5-dihydro-3H,6H-2,2a,5a-triazaaceanthrylen-6-one;
10-(1-((6-chloro-2-(2-methyl-1-oxoisoindolin-5-yl)pyridin-3-yl)amino)-2-hydroxyethyl)-8-methyl-4,5-dihydro-3H,6H-2,2a,5a-triazaaceanthrylen-6-one; and
9-(1-((6-chloro-2-(1-methyl-1H-pyrazol-4-yl)pyridin-3-yl)amino)-2-(4-methylpiperazin-1-yl)ethyl)-3-ethyl-4,7-dimethyl-3,4-dihydro-5H-pyrazolo[3,4-c]isoquinolin-5-one;
or a pharmaceutically acceptable salt thereof.

69. A pharmaceutical composition comprising a compound of claim 1, or a pharmaceutically acceptable salt thereof, and a pharmaceutically acceptable carrier.

70. A method of inhibiting PI3Kα, comprising administering to a subject the compound of claim 1, or a pharmaceutically acceptable salt thereof.

71. The method of claim 70, or a pharmaceutically acceptable salt thereof, wherein the method selectively inhibits PI3Kα over other PI3K isoforms.

72. The method of claim 70, or a pharmaceutically acceptable salt thereof, wherein the method selectively inhibits mutant PI3Kα over wide-type PI3Kα.

73. A method of treating a disease, disorder, or condition associated with PI3Kα, comprising administering to a subject in need thereof the compound of claim 1, or a pharmaceutically acceptable salt thereof.

74. The method of claim 73, wherein the disease, disorder, or condition is associated with mutant PI3Kα.

75. A method of treating cancer, comprising administering to a subject in need thereof the compound of claim 1, or a pharmaceutically acceptable salt thereof.

76. The method of claim 75, wherein the cancer is selected from breast cancer, brain cancer, prostate cancer, endometrial cancer, gastric cancer, leukemia, lymphoma, sarcoma, colorectal cancer, lung cancer, ovarian cancer, skin cancer, and head and neck cancer.

77. The method of claim 76, wherein the disease or disorder is CLOVES syndrome (congenital lipomatous overgrowth, vascular malformations, epidermal naevi, scoliosis/skeletal and spinal syndrome), or PIK3CA-related overgrowth syndrome (PROS).
